(19) United States
(12) Patent Application Publication
(10) Pub. No.: US 2024/0392389 A1
(43) Pub. Date: Nov. 28, 2024

(54) DNA BASED IDENTIFICATION OF SEAFOOD SPECIES IN SAMPLES

(71) Applicants: FFoQSI GmbH, Tulln (AT); Österreichische Agentur für Gesundheit und Ernährungssicherheit GmbH, Vienna (AT); LVA GmbH, Klosterneuburg (AT)

(72) Inventors: Kristina GENSE, Vienna (AT); Verena PETERSEIL, Vienna (AT); Alma LICINA, Klosterneuburg (AT)

(21) Appl. No.: 18/704,597

(22) PCT Filed: Oct. 25, 2022
(86) PCT No.: PCT/EP2022/079764

§ 371 Date: Apr. 25, 2024

(30) Foreign Application Priority Data
Oct. 25, 2021 (EP) 21204456.4

(51) Int. Cl. C12Q 1/6888

(52) U.S. Cl. CPC C12Q 1/6888; C12Q 2600/16

(57) ABSTRACT

A method for identifying seafood species in a sample, comprising the steps of a) isolating DNA from the sample, b) amplifying fragments of the isolated DNA with one or more primer sets, selected from the group of ps 1 for identifying seafood species of the family of Crustacean, ps 2 for identifying seafood species of the family of Cephalopods, ps 3 for identifying seafood species of the family of Gastropoda, ps 4 for identifying seafood species of the family of Veneridae, ps 5 for identifying seafood species of the family of Ostreidae, ps 6 for identifying seafood species of the family of Pectinidae, and ps 7 for identifying seafood species of the family of Mytilidae, c) sequencing the amplified DNA fragments of step b), and d) identifying the seafood species by comparison of the sequences obtained by steps a) to c) with reference sequences of seafood species. Further provided is a primer library and a kit.

### FIELD OF THE INVENTION

The present invention relates to the technical field of DNA barcoding, particularly to a method for the identification of seafood species in samples comprising the steps of isolating DNA from a sample, amplifying fragments of the isolated DNA using specific primers, sequencing of the amplified DNA fragments, and identifying the seafood species through sequence comparison with reference sequences. Further provided herein is a primer library and a kit.

### BACKGROUND OF THE INVENTION

Food adulteration is a worldwide problem in various food products, e.g., in farm animal, wild animal, seafood, and also plant products. The term of food adulteration is not uniformly defined but in general, it describes misdeclaration of food intending to gain an economic benefit without limits (Robson, K. et al, 2021). Seafood has a high risk of fraud and seafood products are often mislabelled. According to Pardo, M. et al. (2016), up to 27% of the seafood is mislabelled worldwide. Food adulteration includes, but is not limited to, replacement (a (valuable) ingredient is replaced by one of a lower value), relabelled or incorrectly labelled food. Incorrect labelling can result when different local names are used for the same species, when the same name is used for different species, or due to translation errors.

However, correct labelling of seafood products is important for traceability issues, protection of endangered species, mitigation of illegal fishing, and for individual reasons of end consumers (Rodriguez, E. M. and Ortea, I., 2017).

Correct declaration of seafood is regulated in the European Union. Thereby, international and national regulations exist to ensure legal trade in seafood and seafood products. The EU directive 1379/2013 regulates market organization of fishery and aquaculture products, including correct declaration of seafood. To comply with legal regulations, labels must include both the local trade name in the official language(s) and the correct scientific Latin name (Regulation (EU) No 1379/2013; Regulation (EU) No 1169/2011).

Regardless of clear and strict requirements for species declaration, incorrect labelling of e.g., bivalve products, has repeatedly been detected in Europe (Naumann, G. et al., 2012; Fernandes, T. et al., 2020). In German and Swiss studies, more than half of the products declared to contain “Jakobsmuschel” (or “Jacobsmuschel”) were labelled incorrectly. Although the German name “Jakobsmuschel” (or “Jacobsmuschel”) may only be used for scallop species of the genus Pecten, species of other genera (particularly Placopecten and Mizuhopecten) were identified in these products (Naumann, G. et al., 2012; Stephan, R. et al., 2014).

Compliance with regulations is especially important since seafood is gaining importance in human nutrition. In 2019, 107.6 billion US $ were made with the marketing of seafood (crustaceans and molluscs), compared to 8.1 billion US $30 years ago. In 2019, 1.03 million tons of mussels, scallops, and oysters were caught in nature and more than 10 million tons were cultivated in aquaculture, earning a profit of millions of US dollars. Worldwide, 6.1 million tonnes of crustaceans were caught and 10.5 million tonnes were cultivated in 2019. In the same year, 6.4 million tonnes of molluscs were caught and 17.6 million tonnes were cultivated.

Crustaceans and molluscs are divided into numerous genera comprising a high number of species with a worldwide distribution. A class of molluscs are for example bivalves, wherein Mytilidae (mussels), Pectinidae (scallops), and Ostreidae (oysters) are the most important bivalve species for human consumption. Each of these bivalve species is divided into several genera comprising a high number of species which makes correct identification of seafood species difficult using known methods.

In the case of seafood, especially for bivalves, morphological characteristics such as shell, colour and size may allow correct species classification. However, after shell removal or mechanical processing, classification by morphology may be hampered or even be impossible (Espiheira, M. et al., 2009; Fernandez, A. et al., 2000).

Recently, matrix-assisted laser desorption ionization time of flight mass spectrometry (MALDI-TOF MS) has been shown to be suitable for accurate species identification of scallops (Stephan, R. et al., 2014). However, MALDI-TOF MS instruments are expensive and do not allow high throughput analysis. Therefore, this methodology is less applicable for routine analyses and for the fast identification of several seafood species.

DNA metabarcoding methods have been recently developed for the identification of mammalian and poultry species in food (Dobrovolny S. et al., 2019).

JP 2010004890 discloses primers and methods for detecting mackerel, salmon, abalone, squid, crab, and shrimp.

Marin A. et al. (2015) disclose the use of Pectinidae family-specific primers for amplifying a partial region at the 5′ end of the 16S rRNA gene as a barcoding tool for scallops.

Marin A. et al. (2017) disclose a decaplex PCR assay for the detection of scallop species with species-specific primers targeting the variable 5′ end of the 16S rRNA gene.

Wen J. et al. (2017) discuss the species characterization of cephalopod products by DNA barcoding and phylogenetic analysis using CO/and 16SrRNA genes.

Spielmann G. et al. (2019) disclose the comparison of three DNA marker regions for their suitability to identify food relevant crustaceans of the order Decapoda.

Sun S. et al. (2021) describe the application of DNA barcoding for the identification of dried shellfish products such as scallop, squid, octopus, and cuttlefish.

Klapper R. et al. (2021) disclose the identification of commercial scallop species through multiplex real-time PCR.

Gense K. et al. (2021) disclose a DNA metabarcoding method for the identification of bivalve species in seafood products.

However, methods which provide comprehensive information on the plurality of seafood species which are present in a food sample are still missing. Thus, there is an urgent need in the field for improved means of identifying several different seafood species in complex and processed foodstuffs suitable for food authentication in routine analysis.

### SUMMARY OF THE INVENTION

It is the objective of the present invention to provide improved means and methods for the identification of several different seafood species in food samples.

The objective is solved by the subject matter of the present invention.

The present invention provides a method which is highly suitable for the identification of seafood species of different origin and processing degree in complex food samples. The inventors of the present invention surprisingly discovered a library of primers which can be incorporated in a fast and reliable metabarcoding method for the identification of a plurality of seafood species in food samples. Thereby, the specific primer sequences of the library are found particularly suitable for combining the primers in the amplification step since they are suitable for amplification at similar conditions such as at similar or even identical temperatures. Temperatures used in a PCR reaction highly depend on the specific characteristics of the primer sequences used in the reaction and may vary even for slightly altered primer sequences. For example, primers with different melting temperatures can hardly be combined successfully in one PCR reaction as the annealing temperature of the PCR reaction depends on the melting temperature of the used primers. Another advantage of the invention described herein is that the method is highly specific based on the specific primer sequences and the length of the primers. This invention successfully identifies seafood species with a low number of PCR cycles, even in highly processed food. The low PCR cycles decrease unspecific PCR results and thus lead to highly reliable results, especially by preventing false positive results.

According to the invention, there is provided a method for identifying seafood species in a sample, comprising the steps of
a) isolating DNA from the sample,
b) amplifying fragments of said DNA with one or more primer sets (ps) selected from the group of
   i. ps 1 for identifying seafood species of the family of Crustacean comprising one or more primer pairs of one forward primer selected from any one of SEQ ID NOs: 1 and 2, and the reverse primer SEQ ID NO: 15 and/or the reverse complement sequences of the primer sequences;
   ii. ps 2 for identifying seafood species of the family of Cephalopods comprising one or more primer pairs of one forward primer selected from any one of SEQ ID NOs: 3 to 5, and the reverse primer SEQ ID NO: 16 and/or reverse complement sequences of the primer sequences;
   iii. ps 3 for identifying seafood species of the family of Gastropoda comprising one or more primer pairs of the forward primer SEQ ID NO: 6, and one reverse primer selected from any one of SEQ ID NOs: 17 to 18 and/or reverse complement sequences of the primer sequences;
   iv. ps 4 for identifying seafood species of the family of Veneridae comprising one or more primer pairs of one forward primer selected from any one of SEQ ID NOs: 7 to 11, and one reverse primer selected from any one of SEQ ID NOs: 19 to 21 and/or reverse complement sequences of the primer sequences;
   v. ps 5 for identifying seafood species of the family of Ostreidae comprising the forward primer SEQ ID NO: 12 and the reverse primer SEQ ID NO: 22 and/or reverse complement sequences of the primer sequences;
   vi. ps 6 for identifying seafood species of the family of Pectinidae comprising the forward primer SEQ ID NO: 13 and the reverse primer SEQ ID NO: 23 and/or reverse complement sequences of the primer sequences; and
   vii. ps 7 for identifying seafood species of the family of Mytilidae comprising one or more primer pairs of the forward primer SEQ ID NO: 14, and one reverse primer selected from any one of SEQ ID NOs: 24 to 25 and/or reverse complement sequences of the primer sequences,
c) sequencing the amplified DNA fragments of step b), and
d) identifying the seafood species by comparison of the sequences obtained by steps a) to c) with reference sequences of seafood species.

Specifically, amplifying of fragments of DNA in step b) is performed by a polymerase chain reaction (PCR), preferably by a PCR comprising 25-30 cycles, more preferably by 25 cycles, and preferably at an annealing temperature of 60-65° C., more preferably at an annealing temperature of 62° C.

Specifically, amplifying fragments of the DNA in step b) is performed with at least 1, 2, 3, 4, 5, 6, or 7 primer sets.

Specifically, the amplified DNA fragments of step b) are 16S rDNA fragments, preferably the amplified DNA fragments comprise 120 bp-220 bp of the 16S rDNA.

Specifically, the reference sequences of seafood species comprise the DNA sequence of the 16S rDNA of said seafood species.

More specifically, the reference sequences are any one of SEQ ID NOs: 28 to 1153, or any combinations thereof.

Specifically, the identified species of the family of Crustacean are selected from the following Group 1: Varuna litterata, Hemisquilla ensigera, Gonodactylus smithii, Pullosquilla thomassini, Chorisquilla trigibbosa, Telmessus acutidens, Lithodes aequispinus, Panulirus echinatus, Jasus paulensis, Jasus caveorum, Parastacus pilimanus, Parastacus brasiliensis, Parastacus defossus, Parastacus nicoleti, Gonodactylus graphurus, Jasus lalandii, Lopholithodes mandtii, Lithodes spp., Lithodes maja, Jasus edwardsii, Panulirus regius, Panulirus pascuensis, Panulirus laevicauda, Panulirus gracilis, Panulirus guttatus, Panulirus femoristriga, Chionoecetes spp., Paralomis granulosa, Panulirus spp., Scyllarus arctus, Palinurus elephas, Episesarma mederi, Austropotamobius torrentium, Cycloachelous granulatus, Eriocheir recta, Cervimunida johni, Achelous floridanus, Portunus sayi, Portunus anceps, Palinurus mauritanicus, Palinurus charlestoni, Pseudosquilla ciliata, Pleuroncodes monodon, Portunus ventralis, Achelous spinicarpus, Callinectes toxotes, Callinectes danae, Callinectes ornatus, Callinectes marginatus, Callinectes affinis, Callinectes rathbunae, Callinectes bocourti, Callinectes similis, Callinectes bellicosus, Callinectes arcuatus, Metanephrops armatus, Metanephrops mozambicus, Metanephrops japonicus, Metanephrops spp., Metanephrops binghami, Parastacus pugnax, Paranephrops zealandicus, Callinectes exasperatus, Palinurus spp., Sagmariasus verreauxi, Metanephrops rubellus, Metanephrops challengeri, Metanephrops neptunus, Metanephrops australiensis, Metanephrops arafurensis, Metanephrops boschmai, Metanephrops formosanus, Metanephrops sinensis, Lithodes ferox, Oratosquillina interrupta, Odontodactylus japonicus, Miyakella nepa, Erugosquilla woodmasoni, Clorida decorata, Dictyosquilla foveolata, Anchisquilla fasciata, Scyllarides herklotsii, Astacus astacus, Portunus hastatus, Achelous ordwayi, Carcinus maenas, Portunus inaequalis, Astacoides madagascariensis, Erimacrus isenbecki, Hemisquilla australiensis, Austrosquilla tsangi, Fallosquilla fallax, Echinosquilla guerinii, Coronis scolopendra, Chorisquilla tweediei, Chorisquilla hystrix, Chorisquilla excavate, Busquilla plantei, Alima pacifica, Alima orientalis, Alachosquilla vicina, Gonodactylellus espinosus, Gonodactylellus affinis, Kempella mikado, Hemisquilla californiensis, Haptosquilla trispinosa, Haptosquilla glyptocercus, Gonodactylus platysoma, Gonodactylaceus falcatus, Gonodactylus childi, Gonodactylellus annularis, Odontodactylus scyllarus, Odontodactylus latirostris, Odontodactylus havanensis, Odontodactylus cultrifer, Neogonodactylus oerstedii, Neogonodactylus bredini, Neogonodactylus bahiahondensis, Lysiosquillina sulcata, Squilla rugosa, Raoulserenea spp., Raoulserenea oxyrhyncha, Pseudosquillopsis marmorata, Raoulserenea komaii, Protosquilla folini, Ibacus alticrenatus, Scyllarides nodifer, Scyllarides haanii, Scyllarides brasiliensis, Taku spinosocarinatus, Jasus frontalis, Procambarus paeninsulanus, Puerulus sewelli, Panulirus polyphagus, Panulirus longipes., Panulirus interruptus, Panulirus marginatus, Ibacus peronii, Ibacus chacei, Faxonella clypeata, Fallicambarus kountzeae, Arenaeus mexicanus, Cambarus tartarus, Chionoecetes tanneri, Thenus unimaculatus, Thenus indicus, Haptosquilla hamifera, Lithodes turritus, Bouchardina robisoni, Troglocambarus maclanei, Hobbseus yalobushensis, Hobbseus prominens, Charybdis spp., Hobbseus petilus, Faxonella creaseri, Thranita danae., Monomia petrea, Neogonodactylus wennerae, Xiphonectes pseudohastatoides, Gonodactylellus viridis, Gonodactylaceus ternatensis, Belosquilla laevis, Procambarus okaloosae, Procambarus morrisi, Procambarus milleri, Procambarus mancus, Procambarus lunzi, Hobbseus cristatus, Procambarus acutissimus, Faxonius pagei, Manningia pilaensis, Pontastacus leptodactylus, Procambarus zonangulus, Procambarus youngi, Procambarus seminolae, Procambarus pycnogonopodus, Procambarus orcinus, Procambarus pallidus, Alima maxima, Scyllarides deceptor, Monomia argentata, Xiphonectes pulchricristatus, Paralithodes platypus, Lopholithodes foraminatus, Faughnia formosae, Faughnia profunda, Bathysquilla crassispinosa, Eriocheir sinensis, Harpiosquilla harpax, Callinectes sapidus, Squilla mantis, Portunus trituberculatus, Panulirus japonicus, Cancer pagurus, Chionoecetes japonicus, Scylla tranquebarica, Scylla serrata, Eriocheir japonica, Eriocheir hepuensis, Cherax destructor, Squilla empusa, Lysiosquillina maculata, Gonodactylus chiragra, Panulirus homarus, Homarus americanus, Panulirus ornatus, Oratosquilla oratoria, Panulirus stimpsoni, Charybdis japonica, Scylla paramamosain, Scylla olivacea, Cherax quadricarinatus, Cherax cainii, Paralithodes brevipes, Paralithodes camtschaticus, Scyllarides latus, Procambarus clarkii, Procambarus fallax, Homarus gammarus, Thenus orientalis, Lithodes nintokuae, Cherax cairnsensis, Cherax dispar, Cherax quinquecarinatus, Cherax robustus, Cherax monticola, Cherax glaber, Cherax holthuisi, Astacopsis gouldi, Portunus pelagicus, Paranephrops planifrons, Nephrops norvegicus, Ibacus ciliatus, Charybdis feriata, Metanephrops sibogae, Panulirus cygnus, Metanephrops thomsoni, Faxonius limosus, Squilloides leptosquilla, Cherax bicarinatus, Austropotamobius pallipes, Cherax tenuimanus, Cherax boesemani, Charybdis (Charybdis) natator, Procambarus acutus, Pacifastacus leniusculus, Munida gregaria, Panulirus versicolor, Faxonius rusticus, Portunus sanguinolentus, Procambarus alleni, Metacarcinus magister, Puerulus angulatus, Lupocycloporus gracilimanus, Monomia gladiator, Varuna yui, Panulirus argus, Munida isos, Scyllarides squammosus, Cambaroides similis, Charybdis bimaculata, Cambarus robustus, Thalamita sima, Thranita crenata, Orconectes luteus, Orconectes punctimanus, Orconectes sanbornii, Cherax spp., Cherax crassimanus, Cherax preissii, Munida spinosa, Munida asprosoma, Munida leagora, Munida alonsoi, Munida taenia, Munida gordoae, Munida zebra, Munida distiza, Munida psamathe, Munida thoe, Munida guttata, Munida stia, Munida ommata, Munida roshanei, Munida compressa, Munida clinata, Munida chydaea, Munida compacta, Munida eclepsis, Munida tyche, Munida philippinensis, Munida armilla, Munida mesembria, Munida spilota, Munida benguela, Munida endeavourae, Munida agave, Munida idyia, Munida militaris, Munida flinti, Munida congesta, Munida rubridigitalis, Munida iris, Munida microphthalma, Munida rufiantennulata, Munida pusilla, Munida remota, Munida leptosyne, Munida rosula, Munida munin, Munida valida, Munida proto, Enriquea leviantennata, Munida multilineata, Munida pagesi, Munida stomifera, Munida quadrispina, Munida tiresias, Munida psylla, Munida heteracantha, Paralomis formosa, Paralomis spinosissima, Paralomis birsteini, Paralomis hirtella, Scyllarus subarctus, Scyllarus pygmaeus, Scyllarus chacei, Scyllarus caparti, Scyllarus americanus, Episesarma palawanense, Episesarma singaporense, Austropotamobius fulcisianus orientalis, Achelous tumidulus, Achelous asper, Achelous sebae, Portunus acuminatus, Achelous tuberculatus, Achelous iridescens, Portunus xantusii, Achelous depressifrons, Achelous rufiremus, Achelous gibbesii, Portunus minimus, Achelous stanfordi, Achelous brevimanus, Portunus affinis, Achelous angustus, Achelous binoculus, Oratosquillina inornata, Oratosquillina asiatica, Oratosquillina anomala, Oratosquillina perpensa, Erugosquilla graham, Busquilla quadraticauda, Kempella stridulans, Gonodactylaceus graphurus, Gonodactylaceus randalli, Carcinus aestuarii, Menippe rumphii, Menippe nodifrons, Menippe spp., Procambarus liberorum, Procambarus toltecae, Procambarus curdi, Procambarus digueti, Procambarus nigrocinctus, Procambarus versutus, Procambarus gibbus, Cambarus pecki, Procambarus geminus, Charybdis acuta, Creaserinus fodiens, Fallicambarus jeanae, Creaserinus gordoni, Creaserinus caesius, Fallicambarus dissitus, Creaserinus danielae, Fallicambarus oryktes, Fallicambarus byersi, Creaserinus burrisi, Creaserinus gilpini, Fallicambarus harpi, Fallicambarus macneesei, Fallicambarus petilicarpus, Fallicambarus wallsi, Fallicambarus strawni, Fallicambarus devastator, Fallicambarus houstonensis, Fallicambarus hortoni, Arenaeus cribrarius, Cambarus spp., Cambarus deweesae, Cambarus striatus, Cambarus graysoni, Cambarus monongalensis, Cambarus pyronotus, Cambarus maculatus, Cambarus latimanus, Cambarus strigosus, Cambarus parrishi, Cambarus bouchardi, Cambarus fasciatus, Cambarus harti, Cambarus nerterius, Cambarus setosus, Cambarus batchi, Cambarus halli, Cambarus unestami, Cambarus reburrus, Cambarus gentry, Cambarus hubbsi, Cambarus friaufi, Cambarus obeyensis, Cambarus cracens, Cambarus asperimanus, Cambarus hobbsorum, Cambarus williami, Cambarus howardi, Cambarus obstipus, Cambarus girardianus, Cambarus cryptodytes, Cambarus sciotensis, Cambarus georgiae, Cambarus pristinus, Cambarus aculabrum, Cambarus englishi, Cambarus brachydactylus, Cambarus cumberlandensis, Cambarus dubius, Cambarus reflexus, Cambarus scotti, Cambarus longirostris, Cambarus hubrichti, Monomia lucida, Faughnia serenei, Harpiosquilla melanoura, Harpiosquilla annandalei, Cherax cuspidatus, Cherax paniaicus, Cherax lorentzi, Cherax albertisii, Cherax rotundus, Cherax leckii, Cherax murido, Cherax wasselli, Cherax parvus, Cherax pallidus, Cherax cartalacoolah, Cherax rhynchotus, Cherax pulcher, Cherax peknyi, Cherax setosus, Cherax misolicus, Cherax warsamsonicus, Cherax snowden, Cherax boschmai, Cherax nucifraga, Cherax barrette, Oratosquilla fabricii, Astacopsis tricornis, Thalamita admete, Faxonius virilis, Thranita prymna, Astacopsis franklinii, Cambaroides schrenckii, Orconectes australis, Thalamita chaptalii, Zygita longifrons, Thalamita picta, Thalamita seurati, Thranita pelsarti, Orconectes barri, Faxonius ronaldi, Faxonius neglectus, Orconectes compressus, Orconectes forceps, Orconectes pellucidus, Neoeriocheir leptognathus, Penaeus kerathurus, Penaeus marginatus, Penaeus longistylus, Penaeus plebejus, Metapenaeopsis liui, Metapenaeopsis lamellata, Metapenaeopsis acclivis, Metapenaeopsis commensalis, Atypopenaeus stenodactylus, Aristeus antillensis, Solenocera vioscai, Penaeus chinensis, Penaeus spp., Metapenaeopsis barbata, Penaeus esculentus, Heteropenaeus longimanus, Atypopenaeus dearmatus, Funchalia taaningi, Xiphopenaeus kroyeri, Trachypenaeopsis mobilispinis, Rimapenaeus similis, Parapenaeus politus, Solenocera membranacea, Alcockpenaeopsis hungerfordii, Batepenaeopsis tenella, Pandalus platyceros, Metapenaeus moyebi, Metapenaeus joyneri, Pandalus montagui, Penaeus brasiliensis, Aristeus antennatus, Heterocarpus laevigatus, Heterocarpus lepidus, Funchalia villosa, Hemipenaeus carpenter, Mesopenaeus tropicalis, Pelagopenaeus balboae, Penaeus hathor, Metapenaeopsis provocatoria, Aristeus virilis, Aristeus alcock, Penaeus aztecus, Heterocarpus abulbus, Penaeus setiferus, Cerataspis monstrosus, Pleoticus robustus, Aristaeopsis edwardsiana, Solenocera necopina, Parapenaeus cayrei, Parapenaeus fissurus, Parapenaeus investigatoris, Parapenaeus fissuroides, Parapenaeus americanus, Heterocarpus ensifer, Kishinouyepenaeopsis cornuta, Parapenaeus perezfarfantae, Parapenaeus murrayi, Parapenaeus longipes, Parapenaeus spp., Heterocarpus chani, Heterocarpus sibogae, Heterocarpus dorsalis, Metapenaeopsis andamanensis, Metapenaeopsis coniger, Macrobrachium idella, Trachysalambria brevisuturae, Trachysalambria aspera, Trachysalambria albicoma, Euphausia superba, Solenocera hextii, Hymenopenaeus equalis, Rimapenaeus constrictus, Crangon crangon, Trachypenaeus anchoralis, Megokris spp., Trachysalambria longipes, Trachysalambria starobogatovi, Trachysalambria nansei, Trachysalambria malaiana, Trachysalambria spp., Trachysalambria parvispina, Crangon uritai, Pandalus borealis, Metapenaeus monoceros, Pandalus nipponensis, Hadropenaeus lucasii, Ganjampenaeopsis uncta, Solenocera annectens, Solenocera melantho, Parapenaeopsis stylifera, Penaeus japonicus, Penaeus brevirostris, Penaeus notialis, Penaeus duorarum, Penaeus schmitti, Artemesia longinaris, Penaeus subtilis, Penaeus stylirostris, Penaeus vannamei, Macrobrachium rosenbergii, Penaeus monodon, Pandalus hypsinotus, Heterocarpus spp., Pandalus jordani, Macrobrachium bullatum, Penaeus merguiensis, Metapenaeus ensis, Acetes chinensis, Macrobrachium nipponense, Penaeus californiensis, Macrobrachium lanchesteri, Pleoticus muelleri, Metapenaeus affinis, Hymenopenaeus neptunus, Penaeus indicus, Aristaeomorpha foliacea, Solenocera spp., Mierspenaeopsis hardwickii, Penaeus latisulcatus, Penaeus semisulcatus, Penaeus isabelae, Sicyonia lancifer, Metapenaeopsis dalei, Metapenaeopsis gerardoi, Parapenaeus longirostris, Pandalus eous, Pandalus miyakei, Pandalus japonicas, Pandalus glabrus, Pandalus teraoi, Pandalus ivanovi, Pandalus coccinatus, Pandalus formosanus, Pandalus chani, Pandalus spp., Pandalus longirostris, Pandalus latirostris, Metapenaeus spp., Palaemon spp., Palaemon serratus, Macrobrachium nipponense, 15 Palaemon capensis, Palaemon sinensis, Palaemon annandalei, Palaemon gravieri, Palaemon serenus, Palaemon carinicauda, Palaemon pugio, Palaemon pandaliformis, Palaemon elegans, Palaemon longirostris, Palaemon peringueyi, Palaemon debilis, Palaemon carteri, Palaemon ritteri, Palaemon orientis, Macrobrachium gracilirostre, Palaemon vulgaris, Palaemon serrifer, Palaemon varians, Palaemon macrodactylus, Palaemon tonkinensis, Palaemon xiphias, Palaemon ivonicus, Palaemon pacificus, Palaemon atrinubes, Palaemon intermedius, Palaemon concinnus, Palaemon yuna, Palaemon antennarius, Palaemon dolospinus, Palaemon gracilis, Palaemon mundusnovus, Palaemon suttkusi, Palaemon zariquieyi, Macrobrachium australiense, Palaemon semmelinkii, Palaemon litoreus, Palaemon septemtrionalis, Palaemon guangdongensis, Palaemon hancocki, Palaemon vietnamicus, Palaemon texanus, Palaemon ortmanni, Palaemon turcorum, Palaemon kadiakensis, Macrobrachium asperulum, Macrobrachium australe, Macrobrachium olfersii, Macrobrachium jelskii, Macrobrachium villosimanus, Macrobrachium equidens, Macrobrachium potiuna, Macrobrachium malcolmsonii, Macrobrachium superbum, Macrobrachium striatum, Macrobrachium latidactylus, Macrobrachium hancocki, Macrobrachium acanthurus, Macrobrachium inflatum, Macrobrachium crenulatum, Macrobrachium carcinus, Macrobrachium americanum, Macrobrachium latimanus, Macrobrachium mammillodactylus, Macrobrachium faustinum, Macrobrachium heterochirus, Macrobrachium scabriculum, Macrobrachium digueti, Macrobrachium tenellum, Macrobrachium idae, Macrobrachium formosense, Macrobrachium dienbienphuense, Macrobrachium placidulum, Macrobrachium sintangense, Macrobrachium niphanae, Macrobrachium totonacum, Macrobrachium tuxtlaense, Macrobrachium vicconi, Macrobrachium villalobosi, Macrobrachium amazonicum, Macrobrachium canarae, Macrobrachium tratense, Macrobrachium forcipatum, Macrobrachium hirsutimanus, Macrobrachium borellii, Macrobrachium brasiliense, Macrobrachium aemulum, Macrobrachium handschini, Macrobrachium horstii, Macrobrachium ferreirai, Macrobrachium lanatum, Macrobrachium novaehollandiae, Macrobrachium tolmerum, Macrobrachium iheringi, Macrobrachium saigonense, Macrobrachium nattereri, Macrobrachium aracamuni, Macrobrachium inpa, Macrobrachium depressimanum, Macrobrachium surinamicum, Macrobrachium denticulatum, Macrobrachium pilimanus, Macrobrachium ohione, Macrobrachium hainanense, Macrobrachium lepidactyloides, Macrobrachium jaroense, Macrobrachium esculentum, Macrobrachium maculatum, Macrobrachium edentatum, Macrobrachium grandimanus, Macrobrachium malayanum, Macrobrachium meridionale, Macrobrachium neglectum, Macrobrachium platycheles, Macrobrachium naso, Macrobrachium placidum, Macrobrachium yui, Macrobrachium shokitai, Macrobrachium sundaicum, Macrobrachium rude, Macrobrachium lamarrei, Macrobrachium sankolli, Macrobrachium gangeticum, Trachysalambria palaestinensis, Euphausia pacifica, Euphausia lucens, Euphausia vallentini, Euphausia triacantha, Euphausia longirostris, Euphausia similis, Euphausia recurve, Euphausia krohni, Euphausia frigida, Euphausia gibboides, Euphausia eximia, Euphausia americana, Euphausia tenera, Euphausia pseudogibba, Euphausia hemigibba, Euphausia brevis, Hymenopenaeus debilis and Nematopalaemon tenuipes (Group 1).

More specifically, the identified species of the family of Crustacean are selected from Group 1 and are listed in Group 1A: Jasus edwardsii, Metanephrops japonicus, Astacus astacus, Eriocheir sinensis, Cancer pagurus, Chionoecetes opilio, Cherax destructor, Homarus americanus, Paralithodes camtschaticus, Procambarus clarkii, Homarus gammarus, Nephrops norvegicus, Panulirus argus, Penaeus setiferus, Aristaeopsis edwardsiana, Crangon, Pandalus borealis, Metapenaeus monoceros, Penaeus notialis, Penaeus duorarum, Penaeus vannamei, Macrobrachium rosenbergii, Penaeus monodon, Penaeus californiensis, Pleoticus muelleri, Penaeus indicus, and Monomia gladiator.

More specifically, the identified species of the family of Crustacean are selected from Group 1 and are listed in Group 1B: Cancer pagurus, Chionoecetes opilio, Homarus americanus, Paralithodes camtschaticus, Procambarus clarkii, Homarus gammarus, Nephrops norvegicus, Panulirus argus, Crangon, Pandalus borealis, Metapenaeus monoceros, Penaeus vannamei, Macrobrachium rosenbergii, Penaeus monodon, and Pleoticus muelleri.

Specifically, the identified species of the family of Cephalopods are selected from the following Group 2: Loligo forbesii, Nototodarus sloanii, Sepia spp., Sepia lorigera, Sepia pardex, Rossia pacifica, Berryteuthis magister, Eledone massyae, Sepia robsoni, Loligo reynaudii, Doryteuthis (Amerigo) pealeii, Doryteuthis (Amerigo) gahi, Sepiola rondeletii, Adinaefiola ligulata, Sepia smithi, Sepia elliptica, Eledone palari, Eledone moschata, Rossia palpebrosa, Gonatus madokai, Gonatus kamtschaticus, Eledone cirrhosa, Sepia elegans, Rossia bipillata, Sepiola atlantica, Lolliguncula (Lolliguncula) panamensis, Octopus maya, Illex illecebrosus, Nototodarus gouldi, Gonatopsis octopedatus, Illex coindetii, Berryteuthis anonychus, Gonatus fabricii, Lusepiola birostrata, Octopus tetricus, Uroteuthis (Photololigo) sibogae, Doryteuthis (Doryteuthis) pleii, Doryteuthis sanpaulensis, Doryteuthis (Amerigo) surinamensis, Octopus hubbsorum, Macrotritopus defilippi, Octopus insularis, Loliolus (Nipponololigo) sumatrensis, Sepia recurvirostra, Sepia madokai, Sepia kobiensis, Amphioctopus aegina, Sepia officinalis, Sepioteuthis lessoniana, Todarodes pacificus, Octopus vulgaris, Heterololigo bleekeri, Octopus sinensis, Octopus americanus, Narrowteuthis nesisi, Ommastrephes bartramii, Sepiella japonica, Uroteuthis (Photololigo) edulis, Doryteuthis (Amerigo) opalescens, Architeuthis dux, Dosidicus gigas, Sepia esculenta, Amphioctopus fangsiao, Loligo vulgaris, Sepiola spp., Octopus mimus, Octopus spp., Octopus bimaculoides, Uroteuthis (Photololigo) chinensis, Uroteuthis (Photololigo) duvaucelii, Illex argentinus, Sepia aculeata, Sepiella inermis, Sepia lycidas, Sepia latimanus, Sepia apama, Sepia pharaonis, Loliolus (Nipponololigo) beka, Alloteuthis subulata, Nototodarus hawaiiensis, Sepia orbignyana, Sepia papuensis, Rossia macrosoma, Lolliguncula (Lolliguncula) brevis, Lolliguncula (Loliolopsis) diomedeae, Afrololigo mercatoris, Octopus bimaculatus, Octopus cyanea, Callistoctopus ornatus, Enteroctopus megalocyathus, Sasakiopus salebrosus, Octopus berrima, Amphioctopus marginatus, Octopus maorum, Octopus fitchi, Amphioctopus neglectus, Loliolus (Nipponololigo) uyii, Loliolus (Nipponololigo) japonica, Bathyteuthis abyssicola, Semirossia patagonica, Cistopus taiwanicus, Sthenoteuthis oualaniensis, Watasenia scintillans, Gonatopsis okutanii, Uroteuthis (Aestuariolus) noctiluca, Sepioteuthis australis, Sepioteuthis sepioidea, Amphioctopus kagoshimensis, Amphioctopus membranaceus, Amphioctopus exannulatus, Amphioctopus rex and Sepia peterseni (Group 2).

More specifically, the identified species of the family of Cephalopods are selected from Group 2 and are listed in Group 2A: Doryteuthis (Amerigo) gahi, Eledone moschata, Octopus maya, Doryteuthis (Doryteuthis) pleii, Amphioctopus aegina, Sepia officinalis, Octopus vulgaris, Sepiella japonica, Uroteuthis (Photololigo) edulis, Doryteuthis (Amerigo) opalescens, Dosidicus gigas, Loligo vulgaris, Uroteuthis (Photololigo) chinensis, Uroteuthis (Photololigo) duvaucelii, Sepiella inermis, Sepia pharaonis, and Amphioctopus membranaceus.

More specifically, the identified species of the family of Cephalopods are selected from Group 2 and are listed in Group 2B: Doryteuthis (Amerigo) gahi, Doryteuthis (Doryteuthis) pleii, Sepia officinalis, Octopus vulgaris, Sepiella japonica, Uroteuthis (Photololigo) edulis, Dosidicus gigas, Loligo vulgaris, Uroteuthis (Photololigo) chinensis, and Uroteuthis (Photololigo) duvaucelii.

Specifically, the identified species of the family of Gastropoda are selected from the following Group 3: Helix pomatia, Achatina fulica, Helix aspersa, Helix aspersa maxima, Helix thessalica, Helix lucorum, Helix nicaeensis, Achatina reticulata, Helix aperta, Helix albescens, Tyrrhenaria ceratina, Helix vladika, Helix spp., Pleurodonte discolor, Pleurodonte lychnuchus, Erctella mazzullii, Erctella cephalaeditana, Pleurodonte formosa, Helix christophi, Helix nordmanni, Pleurodonte nucleola, Pleurodonte parilis, Gonostomopsis auridens, Caracolus caracollus, Lacteoluna selenina, Cernuella cisalpine, Cochlicella acuta, Disculella maderensis, Dialeuca nemoraloides, Monadenia fidelis, Cepaea nemoralis, Sphincterochila candidissima, Microphysula ingersolli, Helicodonta obvoluta and Cernuella virgata (Group 3).

More specifically, the identified species of the family of Gastropoda are selected from Group 3 and are listed in Group 3A: Helix pomatia, Achatina fulica, Helix aspersa, Helix aspersa maxima, Helix lucorum, and Achatina reticulata.

More specifically, the identified species of the family of Gastropoda are selected from Group 3 and are listed in Group 3B: Helix pomatia and Achatina reticulata.

Specifically, the identified species of the family of Veneridae are selected from the following Group 4: Tridacna mbalavuana, Siliqua alta, Megangulus zyonoensis, Megangulus venulosus, Donax faba, Donax cuneatus, Donax kiusiuensis, Mactra quadrangularis, Ensis ensis, Chamelea gallina, Spisula subtruncata, Polititapes rhomboides, Callista chione, Venerupis corrugata, Polititapes aureus, Venus crebrisulca, Mercenaria campechiensis, Antigona lamellaris, Ameghinomya antiqua, Ameghinomya spp., Callista erycina, Venerupis aspera, Paphia philippiana, Venus casina, Ensis spp., Mactra stultorum, Ensis macha, Siliqua minima, Ensis leei, Polititapes durus, Cerastoderma glaucum, Tridacna spp., Donax longissimus, Solen 5 vaginoides, Venus verrucosa, Ezocallista brevisiphonata, Procardium indicum, Cardium maxicostatum, Cardium costatum, Acanthocardia paucicostata, Acanthocardia echinata, Acanthocardia aculeata, Solen spp., Ruditapes philippinarum, Corculum cardissa, Spisula solida, Scrobicularia plana, Mactra spp., Chamelea striatula, Ensis siliqua, Serripes groenlandicus, Tridacna elongatissima, Tridacna rosewateri, Meretrix lamarckii, Meretrix lusoria, Paphia euglypta, Meretrix spp., Acanthocardia tuberculata, Tridacna maxima, Lutraria rhynchaena, Meretrix lyrata, Arctica islandica, Solen strictus, Paratapes undulatus, Paratapes textilis, Paphia amabilis, Solen grandis, Lutraria maxima, Donax vittatus, Donax variegatus, Donax trunculus, Donax semistriatus, Ruditapes decussatus, Cerastoderma edule, Tridacna squamosa, Mactra chinensis and Mercenaria mercenaria (Group 4).

More specifically, the identified species of the family of Veneridae are selected from Group 4 and are listed in Group 4A: Ensis ensis, Chamelea gallina, Callista chione, Venus verrucosa, Ruditapes philippinarum, Ensis siliqua, Meretrix lyrata, Donax trunculus, and Cerastoderma edule.

More specifically, the identified species of the family of Veneridae are selected from Group 4 and are listed in Group 4B: Ensis ensis, Callista chione, Ruditapes philippinarum, Meretrix lyrata, and Cerastoderma edule.

Specifically, the identified species of the family of Ostreidae are selected from the following Group 5: Magallana bilineata, Magallana gigas, Crassostrea virginica, Magallana spp., Magallana angulata, Magallana sikamea, Magallana ariakensis, Ostrea denselamellosa, Magallana nippona, Ostrea edulis, Crassostrea spp., Crassostrea tulipa, Ostrea angasi, Magallana belcheri, Crassostrea rhizophorae, Talonostrea talonata, Crassostrea corteziensis, Ostrea spp., Ostrea chilensis, Ostrea algoensis, Ostrea megodon, Saccostrea cuccullata, Saccostrea palmula, Saccostrea malabonensis, Saccostrea scyphophilla, Saccostrea kegaki, and Saccostrea spp. (Group 5).

More specifically, the identified species of the family of Ostreidae are selected from Group 5 and are listed in Group 5A: Magallana gigas, Crassostrea virginica, and Ostrea edulis.

Specifically, the identified species of the family of Pectinidae are selected from the following Group 6: Euvola spp., Mimachlamys crassicostata, Gloripallium pallium, Flexopecten glaber, Aequipecten opercularis, Nodipecten nodosus, Scaeochlamys livida, Pecten spp., Talochlamys multistriata, Patinopecten caurinus, Chlamys behringiana, Placopecten septemradiatus, Pecten maximus, Zygochlamys delicatula, Chlamys hastata, Ylistrum japonicum, Talochlamys gemmulata, Zygochlamys patagonica, Argopecten purpuratus, Argopecten irradians, Azumapecten farreri, Mizuhopecten yessoensi, Placopecten magellanicus, Chlamys islandica, Argopecten ventricosus, Mimachlamys varia, Amusium pleuronectes, Mimachlamys sanguinea, Talochlamys dichroa, Mimachlamys gloriosa, Mimachlamys cloacata, Mimachlamys asperrima, Annachlamys striatula, Decatopecten radula, Bractechlamys vexillum, Aequipecten glyptus, Scaeochlamys lemniscata, Chlamys rubida, Karnekampia sulcata, Crassadoma gigantea, and Ylistrum balloti (Group 6).

More specifically, the identified species of the family of Pectinidae are selected from Group 6 and are listed in Group 6A: Aequipecten opercularis, Pecten maximus, Pecten jacobaeus, Zygochlamys patagonica, Argopecten purpuratus, Mizuhopecten yessoensi, and Placopecten magellanicus.

Specifically, the identified species of the family of Mytilidae are selected from the following Group 7: Mytilus spp., Perna perna, Mytilus unguiculatus, Perna viridis, Mytilus californianus, Mytilus trossulus, Mytilus galloprovincialis, Mytilus edulis and Perna canaliculus (Group 7).

More specifically, the identified species of the family of Mytilidae are selected from Group 7 and are listed in Group 7A: Perna perna Mytilus californianus, Mytilus trossulus, Mytilus galloprovincialis, Mytilus edulis and Perna canaliculus.

More specifically, the identified species of the family of Mytilidae are selected from Group 7 and are listed in Group 7B: Mytilus galloprovincialis, Mytilus edulis and Perna canaliculus.

Further provided herein is a kit for identifying seafood species in a sample, comprising one or more primer sets selected from the group of
i. ps 1 for identifying seafood species of the family of Crustacean comprising one or more primer pairs of one forward primer selected from any one of SEQ ID NOs: 1 and 2, and the reverse primer SEQ ID NO: 15 and/or the reverse complement sequences of the primer sequences;
ii. ps 2 for identifying seafood species of the family of Cephalopods comprising one or more primer pairs of one forward primer selected from any one of SEQ ID NOs: 3 to 5, and the reverse primer SEQ ID NO: 16 and/or reverse complement sequences of the primer sequences;
iii. ps 3 for identifying seafood species of the family of Gastropoda comprising one or more primer pairs of the forward primer SEQ ID NO: 6, and one reverse primer selected from any one of SEQ ID NOs: 17 to 18 and/or reverse complement sequences of the primer sequences;
iv. ps 4 for identifying seafood species of the family of Veneridae comprising one or more primer pairs of one forward primer selected from any one of SEQ ID NOs: 7 to 11, and one reverse primer selected from any one of SEQ ID NOs: 19 to 21 and/or reverse complement sequences of the primer sequences;
v. ps 5 for identifying seafood species of the family of Ostreidae comprising the forward primer SEQ ID NO: 12 and the reverse primer SEQ ID NO: 22 and/or reverse complement sequences of the primer sequences;
vi. ps 6 for identifying seafood species of the family of Pectinidae comprising the forward primer SEQ ID NO: 13 and the reverse primer SEQ ID NO: 23 and/or reverse complement sequences of the primer sequences; and
vii. ps 7 for identifying seafood species of the family of Mytilidae comprising one or more primer pairs of the forward primer SEQ ID NO: 14, and one reverse primer selected from any one of SEQ ID NOs: 24 to 25 and/or reverse complement sequences of the primer sequences, optionally further comprising PCR components, buffers, reagents and/or an instruction manual.

Further provided herein is a library of primer sequences comprising any one of SEQ ID NOs: 1 to 25, or any combinations of SEQ ID NOs: 1 to 25.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017); Berg et al, “Stryer Biochemie” Springer Verlag, 2018; and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, expression constructs, transformed host cells and modified proteins and enzymes, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

The method of the present invention relates to the field of DNA barcoding. Compared with existing morphological identification methods, the method of the present invention is not affected by the experience of inspectors or the morphological changes after processing, which greatly improves the feasibility of sample detection. Compared with the existing DNA barcoding techniques, the present invention solves the problem of false negative results in highly processed foods caused by the difficulty in amplifying DNA of bivalve organisms by the existing DNA barcodes. Therefore, the DNA barcode and its application method established by the present invention are important supplements to the existing DNA barcoding techniques, and can be used to simultaneously identify a plurality of seafood species of the family of Crustacean, Cephalopods, Gastropoda, Veneridae, Ostreidae, Pectinidae, and Mytilidae even if present in very low amounts.

The DNA barcoding identification technique is mainly used for species identification by using relatively short DNA fragments in the organism with sufficient variation that can represent and map this species by means of PCR amplification, sequencing and alignment, etc. DNA barcoding aims at detecting a broad range of species by using universal primer systems. DNA metabarcoding allows the identification of multiple species in food samples in one and the same sequencing run.

DNA barcodes commonly contain conserved regions at both ends, serving as binding sites for universal primers, and a variable part in between the primer binding sites, for differentiation between the species of interest. Due to its high copy number and robustness, mitochondrial DNA (mtDNA) is preferred over genomic DNA. The mtDNA regions most commonly used for species identification are cytochrome c oxidase subunit I (COI), cytochrome b (cyt b), and 16S ribosomal DNA (16S rDNA).

According to the invention, seafood species are identified based on a barcoding method. The term “seafood species” refers to species of the following families: Crustacean, Cephalopods, Gastropoda, Veneridae, Ostreidae, Pectinidae, and Mytilidae. In biological classification, the taxonomic rank is the relative level of a group of organisms in a taxonomic hierarchy. As used herein, the following taxonomic ranks are referred to as according to their relative level of a group of organisms in descending taxonomic hierarchy: family, genus, species. As an example, the species Pecten jacobaeus and Placopecten magellanicus belong to the genus Pecten and the genus Pecten belongs to the family of Pectinidae.

According to a specific embodiment, the seafood species identified according to the invention are the species described herein selected from the group of families of Crustacean, Cephalopods, Gastropoda (snails), Veneridae (venus clams), Ostreidae (oysters), Pectinidae (scallops), and Mytilidae (mussels).

The term “sample” refers to samples which may be taken from different seafood, foodstuff, different origin of the foodstuff and various processing degrees of the food. For example, the sample is taken from sauces, soups, seafood mix, chips, pastes, seafood in cans e.g. mussels in various sauces, raw or frozen seafood, or raw or frozen seafood ingredients.

According to one embodiment of the invention, DNA is isolated from a sample. DNA can be isolated from a sample e.g., by using special kits for DNA isolation/extraction or by using the CTAB (ionic detergent cetyltrimethylammonium bromide)-method. The method for isolating DNA according to the invention enables the isolation of DNA from different samples, specifically from food samples.

According to one embodiment of the invention, the amplification of DNA fragments of isolated DNA is performed by PCR (polymerase chain reaction). Variations of PCR may be used, e.g. real time PCR with intercalating dyes.

In another embodiment, in order to improve the PCR amplification according to the invention, concentrations of compounds commonly used in PCR technology may be adapted. For example, the concentration of magnesium chloride, commonly used in PCR amplification assays, may be increased or decreased depending on the specific setup.

In yet another embodiment, PCR methods rely commonly on thermal cycling, wherein the DNA is replicated by repeated cycles of heating and cooling permitting different temperature-dependent reactions. In general, one PCR-cycle comprises the steps of denaturation, annealing, and extension. Accordingly, the PCR method according to the invention comprises 25-30 cycles for the amplification of DNA fragments. Specifically, the PCR method according to the invention comprises 25, 26, 27, 28, 29, or 30 cycles.

In yet another specific embodiment, the PCR method according to the invention comprises an annealing temperature of 60-65° C., specifically 60, 61, 62, 63, 64, or 65° C. More specifically, the annealing temperature is 62° C.

According to one embodiment, in the PCR assay of the invention, DNA fragments of different seafood species are amplified in one single PCR assay due to the usage of multiple primer pairs. Thereby, a primer pair is consisting of one forward and one reverse primer. Multiplex PCR is enabled since multiple primer pairs are used within a single PCR mixture to produce amplicons of different DNA sequences. Thereby, several different DNA sequences can be amplified simultaneously. By amplifying multiple different DNA fragments at once, additional information is gained from a single test-run.

According to one embodiment of the invention, fragments of the 16S rDNA of the mtDNA region are amplified from isolated DNA samples. Thus, the mtDNA region used for seafood species identification is the 16S rDNA.

In a specific embodiment, the 16S rDNA fragments comprise 130 bp to 220 bp. The length of 16S rDNA fragments vary among the seafood species to be identified. The 16S rDNA fragments comprise 190 bp to 220 bp, 150 bp to 160 bp, or 130 bp to 150 bp. Specifically, for species of the family of Crustacean, the 16S rDNA fragments comprise 198-220 bp. Specifically, for species of the family of Cephalopods, the 16S rDNA fragments comprise 194-220 bp. Specifically, for species of the family of Gastropoda, the 16S rDNA fragments comprise 154-157 bp. Specifically, for species of the family of Veneridae, the 16S rDNA fragments comprise 128-149 bp. Specifically, for species of the families of Ostreidae, Pectinidae, and Mytilidae, the 16S rDNA fragments comprise 133-148 bp.

According to the invention, a primer pair consisting of one forward primer and one reverse primer binds to a conserved region at both ends of the 16S rDNA fragment which is targeted to be amplified by PCR. In order to identify the seafood species according to the invention, a PCR product has to be obtained for the species to be identified. Due to high sequence variability between closely related seafood species of certain families, more than one primer pair is needed to obtain a PCR product for all the species of the family.

Specifically, the term “primer set” as used herein refers to the set of all the primer pairs which are needed for the identification of seafood species of a specific family, wherein the families are the following: Crustacean, Cephalopods, Gastropoda, Veneridae, Ostreidae, Pectinidae, and Mytilidae. As an example, three primer sets are needed for the identification of seafood species belonging the three families, Pectinidae, Ostreidae, and Mytilidae and each primer set is specific for one of the families.

More specifically, depending on the specific family, the primer set comprises one primer pair consisting of one forward primer and one reverse primer or comprises more than one primer pair. In the case that the primer set comprises more than one primer pair, each of the primer sequences of the primer set can be used to form multiple primer pairs. For example, if one primer set comprises two forward primer sequences and one reverse primer sequence, two primer pairs are formed. These two primer pairs are formed by selecting one forward primer from the two forward primer sequences and combining the forward primer sequence with the reverse primer sequence.

The herein described primers enable a simultaneous use in one single PCR reaction because of their specific characteristics such as their sequence length. For a simultaneous PCR reaction using different primers and primer pairs, it is important that all primers are suitable for the selected PCR conditions. For example, slight variations of the sequence of a primer (or primer pair) can result in an altered melting temperature which can ultimately lead to an unsuccessful PCR reaction if the annealing temperature of the PCR reaction is not adjusted. For example, using other primers known in the art, such as the primers disclosed in JP 2010004890, could possibly result in no PCR products since such primers are possibly not compatible with the primers described herein concerning e.g., their length, melting temperature or during the following sequencing.

According to one embodiment of the invention, the primers sequences of the primer sets specific for the identification of seafood species of the families Crustacean, Cephalopods, Gastropoda, Veneridae, Ostreidae, Pectinidae, and Mytilidae are given in Table 1. The forward primer SEQ ID NOs: 1 to 14 and reverse primer SEQ ID NOs: 15 to 25 are given in 5′-3′ direction. The reverse complement of the primer sequences can also be used in the method provided herein.

Specifically, primer set (ps) 1 is used for the identification of species of the family of Crustacean. In ps 1, one or more, specifically one or two primer pairs are formed by selecting a forward primer from forward primer sequences SEQ ID NOs: 1 and 2 and combining the selected forward primer with the reverse primer given in reverse primer sequence SEQ ID NO: 15. The primer set for the identification of Crustacean comprises SEQ ID NOs: 1, 2, and 15.

Specifically, ps 2 is used for the identification of species of the family of Cephalopods. In ps 2, one or more, specifically one, two, or three primer pairs are formed by selecting a forward primer from SEQ ID NOs: 3 to 5 and combining the selected forward primer with the reverse primer given in reverse primer sequence SEQ ID NO: 16. The primer set for the identification of Cephalopods comprises SEQ ID NOs: 3, 4, 5, and 16.

Specifically, ps 3 is used for the identification of species of the family of Gastropoda. In ps 3, one or more, specifically one or two primer pairs are formed by forward primer from SEQ ID NO: 6 and combining the forward primer with one reverse primer selected from reverse primer sequences SEQ ID NOs: 17 to 18. The primer set for the identification of Gastropoda comprises SEQ ID NOs: 6, 17, and 18.

Specifically, ps 4 is used for the identification of species of the family of Veneridae. In ps 4, one or more, specifically one, two, three, four, or five primer pairs are formed by selecting a forward primer from SEQ ID NOs: 7 to 11 and combining the selected forward primer with one reverse primer selected from reverse primer sequences SEQ ID NOs: 19 to 21. The primer set for the identification of Veneridae comprises SEQ ID NOs: 7, 8, 9, 10, 11, 19, 20, and 21.

Specifically, ps 5 is used for the identification of species of the family of Ostreidae. In ps 5, one primer pair is formed by the forward primer SEQ ID NO: 12 and reverse primer sequence SEQ ID NO: 22. The primer set for the identification of Ostreidae comprises SEQ ID NOs: 12 and 22.

Specifically, ps 6 is used for the identification of species of the family of Pectinidae. In ps 6, one primer pair is formed by the forward primer SEQ ID NO: 13 and reverse primer sequence SEQ ID NO: 23. The primer set for the identification of Pectinidae comprises SEQ ID NOs: 13 and 23.

Specifically, ps 7 is used for the identification of species of the family of Mytilidae. In ps 7, one or more, specifically one or two primer pairs are formed by the forward primer SEQ ID NO: 14 and combining the forward primer with one reverse primer sequence selected from reverse primer sequences SEQ ID NOs: 24 to 25. The primer set for the identification of Mytilidae comprises SEQ ID NOs: 14, 24, and 25.

Specifically, the amplification of DNA fragments according to the invention is performed with at least 1, 2, 3, 4, 5, 6, or 7 primer sets.

In one embodiment, the concentrations of the primers may be adapted to the specific combination of forward and reverse primers in a primer set for a single species. For example, if the PCR amplification of Mytilidae is performed with two primer pairs, namely primer pair 1 being SEQ ID NO: 14 and SEQ ID NO: 24, and primer pair 2 being SEQ ID NO: 14 and SEQ ID NO: 25, then the concentration in the PCR assay of SEQ ID NO: 14 may be twice as high as the concentration of each SEQ ID NO: 24 and SEQ ID NO: 25.

According to the invention, the amplified sequences are sequenced in order to determine the nucleotide sequence of the amplified 16S rDNA fragment. Specifically, Sanger sequencing or next generation sequencing (NGS) is applied as sequencing technology.

According to the invention, the sequenced DNA fragments are identified based on a comparison with reference sequences of the respective species. Specifically, the variable part in between the primer binding sites is used for differentiation between the species of interest. Methods for sequence comparison are commonly known in the art. Specifically, BLASTn provided by NCBI (National Center for Biotechnology Information) can be used for sequence comparison of a sequenced DNA fragment with a reference sequence database in order to identify the seafood species in the sample. More specifically, the sequences DNA fragments obtained by the method of the invention are compared to reference sequences comprising the 16S rDNA sequence of the targeted seafood species.

According to a specific embodiment, the reference sequences of the invention are selected from any one of SEQ ID NOs: 28 to 1153. More specifically, the sequenced DNA fragments are identified based on a comparison with a database or a library comprising reference sequences of the species identified according to the invention. In this case, the library of reference sequences comprises one or more of SEQ ID NOs: 28 to 1153, or all of SEQ ID NOs: 28 to 1153, or any combinations thereof.

According to one embodiment of the invention, a library of primer sequences is provided herein comprising SEQ ID NOs: 1 to 25 and/or the reverse complement sequences thereof. The sequences of said primer sequences (SEQ ID NOs: 1 to 25) are given in the Table 1. The primer library of the invention may comprise all primers listed in Table 1. Alternatively, the primer library comprises primers of primer set 1, 2, 3, 4, 5, 6, and/or 7.

As used herein, the term “Fwd” refers to a forward and the term “Rev” refers to a reverse primer. Alternatively, also the term “For” may be used as abbreviation for a forward primer.

The nomenclature of the nucleotide sequences of the invention follows the general guidelines of the IUPAC nucleotide code. Specifically, nucleotide code A refers to adenine, C refers to cytosine, G refers to guanine, T refers to thymine, and W refers to A or T.

According to a specific embodiment, if a primer sequence comprises a “W” at a specific position in the nucleotide sequence, said primer is used as a mixture of two sequences, wherein one primer has an adenine at the specific position and the other primer has thymine at the specific position.

TABLE 1

| | | | | SEQ |
|---|---|---|---|---|
| | | | | ID |
| Species | Type | Name | Sequence 5′-3′ | NO |
| Crustacean | Fwd | For_Ga1 | 5′ GGGGGACGATAAGACCCTATAAA 3′ | 1 |
| Crustacean | Fwd | For_Ga4 | 5′ AATGGGAAGACAAGACCCTATAAA 3′ | 2 |
| Crustacean | Rev | Rev_Kr45 | 5′ ATTACGCTGTTATCCCTAAAGTAACTT 3′ | 15 |
| Cephalopods | Fwd | For_Ti1+2 | 5′ GGGACGAGAAGACCCTAWTGA 3′ | 3 |
| Cephalopods | Fwd | For_Ti1+2_A | 5′ GGGACGAGAAGACCCTAATGA 3′ | 4 |
| Cephalopods | Fwd | For_Ti1+2_T | 5′ GGGACGAGAAGACCCTATTGA 3′ | 5 |
| Cephalopods | Rev | Rev_Ti5 | 5′ ATTACGCTGTTATCCCTATGGTAACT 3′ | 16 |
| Gastropoda | Fwd | For_Sc6 | 5′ TGACTGTGCAAAGGTAGCATAAT 3′ | 6 |
| Gastropoda | Rev | Rev_Sc7 | 5′ AAGTTTCTAGGGTCTTCTCGTCT 3′ | 17 |
| Gastropoda | Rev | Rev_Sc8 | 5′ AAACTCTAAGGGTCTTCTCGTCT 3′ | 18 |
| Veneridae | Fwd | For_Mu1 | 5′ TTGGCCTTTAATTGGGGTCC 3′ | 7 |
| Veneridae | Fwd | For_Mu2 | 5′ GGTAGCGCGATAATTTGTCTCTTAA 3′ | 8 |
| Veneridae | Fwd | For_Mu4 | 5′ CTTAATTGGAGAAGGGTATGAATGG 3′ | 9 |
| Veneridae | Fwd | For_Mu7 | 5′ GTTTAACGGCCGCAGTTGTC 3′ | 10 |
| Veneridae | Fwd | For_Mu15 | 5′ TACCGCAGGGATAACAGCG 3′ | 11 |
| Veneridae | Rev | Rev_Mu8 | 5′ GCTCGACAGGGTCTTCTCGTCT 3′ | 19 |
| Veneridae | Rev | Rev_Mu13 | 5′ CAAAAATTCAGGTTTTTTCACTTG 3′ | 20 |
| Veneridae | Rev | Rev_Mu40 | 5′ TCATGTAAGAAATTAAAAAACGAACAG 3′ | 21 |
| Ostreidae | Fwd | For_Mu25 | 5′ GGTAGCGAAATTCCTTGCCTT 3′ | 12 |
| Ostreidae | Rev | Rev_Mu26 | 5′ AAAGTTGCACGGGGTCTT 3′ | 22 |
| Pectinidae | Fwd | For_Mu21 | 5′ TGCTAAGGTAGCTAAATTATGGCC 3′ | 13 |
| Pectinidae | Rev | Rev_Mu23 | 5′ CTTCACGGGGTCTTCTCGTC 3′ | 23 |
| Mytilidae | Fwd | For_Mu27 | 5′ CCTTTTGCATAAGGGTTTTTCAAG 3′ | 14 |
| Mytilidae | Rev | Rev_Mu29 | 5′ CGAATAGTATCTAGCCGCCATTC 3′ | 24 |
| Mytilidae | Rev | Rev_Mu30 | 5′ GCAAATAGCATATCACTTTCACCTC 3′ | 25 |

According to one embodiment of the invention, a kit for identifying seafood species in a sample is provided, wherein the kit comprises the primer library of the invention. Specifically, the kit comprises primer sequences of one or more of the primer sets selected from primer set 1, primer set 2, primer set 3, primer set 4, primer set 5, primer set 6, and primer set 7. More specifically, the kit comprises at least 1, 2, 3, 4, 5, 6, or 7 of the primer sets according to the invention. The kit may also comprise the reverse complement sequences of the primer sequences.

Said kit may further comprise PCR components, buffers, reagents and/or an instruction manual. Specifically, the kit may comprise as PCR components a polymerase, a master mix, and/or magnesium chloride.

### Examples

The examples described herein are illustrative of the present invention and are not intended to be limitations thereon. Many modifications and variations may be made to the techniques described and illustrated herein without departing from scope of the invention. Accordingly, it should be understood that the examples are illustrative only and are not limiting upon the scope of the invention.

### Example 1—Pectinidae, Ostreidae, and Mytilidae

In Example 1, the identification of seafood species of the family of Pectinidae, Ostreidae, and Mytilidae (together known as “bivalves”) in raw and processed food products is described. The method was developed on the Illumina MiSeq® and iSeq® platforms.

### Materials and Methods

### Sample Collection and Storage

86 commercial food products were collected from regional supermarkets, fish markets, and delicacy shops (Table 2). Table 2 shows the declaration, origin and processing condition of the 86 commercial food products. Samples were either fresh, deep-frozen, or in processed condition. Each sample was given a specific ID number, with the letter “0” referring to oysters, “S” to scallops, “M” to mussels, and “Mi” to mixed-species seafood. Samples were stored at −20° C. until DNA extraction.

TABLE 2

| | Declaration on the product | | * | | |
|---|---|---|---|---|---|
| Sample | Scientific/Latin | Product | Product | Purchase | |
| ID | name | description | origin | origin | Treatment |
| O1 | Crassostrea gigas | Giant Oyster | France | delicacy shops | raw |
| O2 | Ostrea edulis | Oyster | Denmark | fish market | raw |
| O3 | Crassostrea gigas | Oyster/Gillardeau | Denmark | fish market | raw |
| O4 | not declared | Oyster | Pacific * | delicacy shops | raw |
| O5 | Crassostrea gigas | Oyster in | Korea | delicacy shops | processed |
| | | sunflower oil | | | |
| O6 | Crassostrea gigas | Oyster in | Korea | regional | processed |
| | | sunflower oil | | supermarket | |
| O7 | Crassostrea gigas | Oyster in water | Korea | delicacy shops | processed |
| O8 | not declared | Oyster sauce | Thailand | regional | processed |
| | | | | supermarket | |
| O9 | not declared | Oyster sauce | Thailand | delicacy shops | processed |
| O10 | not declared | Oyster sauce | China | delicacy shops | processed |
| M11 | Mytilus edulis | Mussel | Spain | delicacy shops | processed |
| M12 | Mytilus | Blue Mussel | Spain | regional | frozen |
| | galloprovincialis | | | supermarket | |
| M13 | Perna canaliculus | New Zealand | New Zealand | delicacy shops | frozen |
| | | green-lipped | | | |
| | | mussel | | | |
| M14 | Mytilus spp. | Blue Mussel | not declared | not declared | frozen |
| M15 | Mytilus edulis | Blue Mussel | North Atlantic * | delicacy shops | raw |
| M16 | Mytilus edulis | Bouchot mussel | France | fish market | raw |
| M17 | not declared | Grilled blue | Italy | delicacy shops | processed |
| | | mussel | | | |
| M18 | Mytilus chilensis | Blue Mussel | Spain | delicacy shops | frozen |
| M19 | not declared | Blue Mussel | not declared | fish market | raw |
| M20 | Mytilus spp. | Blue Mussel | not declared | fish market | raw |
| M21 | Mytilus edulis | Mussel | Denmark | delicacy shops | processed |
| M22 | Mytilus | Blue Mussel | Italy | delicacy shops | processed |
| | galloprovincialis | | | | |
| M23 | not declared | Mussel with | Spain | delicacy shops | processed |
| | | sherry vinegar | | | |
| M24 | Mytilus chilensis | Blue Mussel in | Denmark | regional | processed |
| | | tomato sauce | | supermarket | |
| M25 | not declared | Mussel in | Spain | regional | processed |
| | | marinade sauce | | supermarket | |
| M26 | not declared | Grilled blue | Italy | regional | processed |
| | | mussel | | supermarket | |
| M27 | Mytilus chilensis | Mussel in | Spain | regional | processed |
| | | marinade | | supermarket | |
| M28 | not declared | Dry cat food with | Germany | delicacy shops | processed |
| | | green lipped | | | |
| | | mussel | | | |
| M29 | Mytilus | Blue mussel in | Spain | regional | processed |
| | galloprovincialis | tomato sauce | | supermarket | |
| M30 | Mytilus | Blue mussel a la | Spain | regional | processed |
| | galloprovincialis | mariniere | | supermarket | |
| M31 | not declared | Blue mussel in | Germany | delicacy shops | processed |
| | | organic marinade | | | |
| M32 | not declared | Marinated blue | Spain | delicacy shops | processed |
| | | mussels | | | |
| M33 | Mytilus chilensis | Mussel in | Denmark | delicacy shops | processed |
| | | Escabeche | | | |
| M34 | Mytilus chilensis | Mussel | Denmark | delicacy shops | processed |
| M35 | Mytilus chilensis | Mussel in tomato | Denmark | delicacy shops | processed |
| | | sauce | | | |
| M36 | Mytilus | Blue mussel | Germany | delicacy shops | processed |
| | galloprovincialis | marinated | | | |
| M37 | Mytilus edulis | Mussel in honey | Denmark | delicacy shops | processed |
| | | mustard sauce | | | |
| M38 | not declared | Blue mussel in | Spain | delicacy shops | processed |
| | | marinade | | | |
| M39 | Mytilus chilensis | Blue mussel | Germany | not declared | processed |
| M40 | Mytilus edulis | Blue mussel | Netherlands | not declared | raw |
| S41 | Placopecten | Deep-sea scallop | France | regional | cooled |
| | magellanicus | | | supermarket | |
| S42 | Mizuhopecten | Yesso scallop | North Atlantic | fish market | cooled |
| | yessoensis | | | | |
| S43 | Pecten maximus | Great scallop | Pacific * | delicacy shops | cooled |
| S44 | Pecten spp. | Great scallop | not declared | not declared | frozen |
| S45 | Placopecten | Deep-sea scallop | not declared | not declared | frozen |
| | magellanicus | | | | |
| S46 | Pecten jacobaeus | Great scallop | Croatia | delicacy shops | frozen |
| S47 | Zygochlamys | Scallop “á la | France | delicacy shops | processed |
| | patagonica | Bretonne” | | | |
| S48 | Patinopecten | Great scallop/ | Pacific * | fish market | cooled |
| | yessoensis | Yesso scallop | | | |
| S49 | Placopecten | Great scallop | Pacific * | delicacy shops | cooled |
| | magellanicus | | | | |
| S50 | Argopecten | Pacific scallop | Peru | delicacy shops | frozen |
| | purpuratus | | | | |
| S51 | not declared | Great scallop | not declared | fish market | cooled |
| S52 | Patinopecten | Great scallop | Pacific * | delicacy shops | cooled |
| | yessoensis | | | | |
| S53 | Pecten sp. | Great scallop | not declared | fish market | cooled |
| S54 | Placopecten | Great scallop | Pacific * | delicacy shops | cooled |
| | magellanicus | | | | |
| S55 | Aequipecten | Scallop in sauce | Spain | delicacy shops | processed |
| | opercularis | | | | |
| S56 | not declared | Great scallop | not declared | restaurant | processed |
| S57 | Placopecten | Great scallop | Austria | fish market | cooled |
| | magellanicus | | | | |
| S58 | not declared | Rillettes de | France | delicacy shops | processed |
| | | Saint-Jacques | | | |
| S59 | not declared | Small scallop in | Spain | delicacy shops | processed |
| | | galician sauce | | | |
| S60 | not declared | Deep-sea scallop | Germany | not declared | frozen |
| S61 | Patinopecten | Great scallop | Pacific * | delicacy shops | cooled |
| | yessoensis | | | | |
| Mi62 | Mytilus chilensis | Seafood mix | France | regional | frozen |
| | | | | supermarket | |
| Mi63 | not declared | Sauce with | Italy | regional | processed |
| | | seafood | | supermarket | |
| Mi64 | Mytilus chilensis, | Seafood mix | Germany | delicacy shops | processed |
| | Mytilus edulis | | | | |
| Mi65 | not declared | Bouillabaise | Germany | delicacy shops | processed |
| | | Marsille | | | |
| Mi66 | Mytilus chilensis | Seafood mix | France | regional | processed |
| | | | | supermarket | |
| Mi67 | Mytilus spp. | Seafood mix | Chile | regional | processed |
| | | | | supermarket | |
| Mi68 | Mytilus | Sea fruit salad in | Italy | regional | processed |
| | galloprovincialis | sunflower oil | | supermarket | |
| Mi69 | Mytilus chilensis | Seafood mix | France | delicacy shops | processed |
| Mi70 | not declared | Sea fruit salad | Italy | regional | processed |
| | | fantasy | | supermarket | |
| Mi71 | not declared | Seafood mix | Italy | regional | processed |
| | | | | supermarket | |
| Mi72 | Mytilus chilensis | Seafood mix | Croatia | delicacy shops | processed |
| Mi73 | not declared | Seafood mix | not declared | not declared | unknown |
| Mi74 | not declared | Seafood mix | not declared | not declared | unknown |
| Mi75 | not declared | Pizza Frutti | Austria | restaurant | processed |
| | | di mare | | | |
| Mi76 | not declared | Paella | Germany | delicacy shops | processed |
| Mi77 | Mytilus edulis, | Paella | Germany | regional | processed |
| | Mytilus chilensis | | | supermarket | |
| Mi78 | Mytilus chilensis | Seafood all'Olio | France | regional | processed |
| | | | | supermarket | |
| Mi79 | Mytilus chilensis | Seafood mix | Spain | delicacy shops | processed |
| Mi80 | Mytilus chilensis | Seafood mix | Austria | delicacy shops | processed |
| Mi81 | not declared | Sea fruit salad | Italy | delicacy shops | processed |
| Mi82 | Zygochlamys | Scallop terrine | France | delicacy shops | processed |
| | patagonica, | | | | |
| | Chlamys | | | | |
| | opercularis | | | | |
| Mi83 | not declared | Terrine of salmon | Austria | delicacy shops | processed |
| | | and great scallop | | | |
| Mi84 | Mytilus chilensis | Seafood mix | Germany | delicacy shops | processed |
| Mi85 | not declared | Instant noodle | Korea | delicacy shops | processed |
| | | seafood, mild | | | |
| Mi86 | not declared | Instant noodle | Korea | delicacy shops | processed |
| | | seafood, spicy | | | |

* In case the country of production was unknown, the fishing region was specified

Eleven out of the 86 samples (“reference samples”), comprising three mussel, six scallop, and two oyster species, were used for method development. Table 3 shows the bivalve species used for development of the DNA metabarcoding method. Identity of bivalve species in these reference samples (samples M12, M13 and M27 for mussels; samples S42, S46, S47, S49, S50, and S55 for scallops; samples 02 and 03 for oysters; Table 2) was verified by subjecting DNA extracts to Sanger sequencing (Microsynth, Balgach, Switzerland) and matching the sequences against the public databases provided by the National Center for Biotechnology Information (NCBI).

TABLE 3

| Scientific name | Commercial name (German) | Commercial name (English) |
|---|---|---|
| Mytilidae | Miesmuscheln | mussels |
| Mytilus edulis | Gemeine Miesmuschel | Blue mussel |
| Mytilus galloprovincialis | Mittelmeer-Miesmuschel | Mediterranean mussel |
| Perna canaliculus | Neuseeland-Miesmuschel | New Zealand green-lipped mussel |
| Pectinidae | Kammmuscheln | scallops |
| Placopecten magellanicus | Atlantischer Tiefseescallop | Atlantic deep-sea scallop |
| Mizuhopecten yessoensis | Japanische Kammmuschel | Yesso scallop |
| Pecten jacobaeus | Jakobsmuschel | Great scallop |
| Zygochlamys patagonica | Patagonische Kammmuschel | Patagonian scallop |
| Argopecten purpuratus | Purpur-Kammmuschel | Purple scallop |
| Aequipecten opercularis | Kleine Pilgermuschel | Queen scallop |
| Ostreidae | Austern | oysters |
| Magallana gigas | Pazifische Felsenauster | Pacific oyster |
| Ostrea edulis | Europäische Auster | European flat oyster |

### DNA Extraction and Quantification

Raw material was cut into smaller pieces or homogenized. To 2.0 gram of each sample, 10 mL of a hexadecyltrimethylammonium bromide (CTAB) buffer was added. After addition of 80 μL proteinase K, the mixture was incubated on an Intelli-Mixer™ RM2 (LTF Labortechnik) overnight at 50° C.

For DNA isolation, a commercial kit (Maxwell® 16 FFS Nucleic Acid Extraction System Custom-Kit, Promega, Madison, USA) was used according to the manufacturer's instruction. DNA concentration was determined fluorometrically (Qubit® 2.0 fluorometer, Thermo Fisher Scientific, Oregon, USA). For higher concentrations, the Qubit® dsDNA broad range assay kit (2 to 1000 ng) and for lower concentrations, the Qubit® dsDNA high sensitivity assay kit (0.2 to 100 ng) was used. DNA purity was assessed from the ratio of the absorbance at 260 nm and 280 nm (QlAxpert spectrophotometer, software version 2.2.0.21, Qiagen, Hilden, Germany). DNA extracts were stored at −20° C. until further use.

### DNA Extract Mixtures

Ternary DNA extract mixtures were prepared by mixing DNA extracts (DNA concentration 5 ng/mL) from Pecten spp., Magallana gigas and Mytilus galloprovincialis, representing the three bivalve species Pectinidae, Ostreidae, and Mytilidae, respectively. Individual DNA extracts were mixed in a ratio of 98.0:1.5:0.5 (v/v/v).

In addition, DNA extract mixtures consisting of DNA from species belonging to one bivalve species were prepared. In these mixtures, DNA from one species was present as the main component, DNA from the other species as minor components (1.0% each). Since only two oyster species were available, the DNA extract mixture representing the bivalve species Ostreidae contained the closely related scallop (Placopecten magellanicus) as major component (98.0%) and DNA from the two oyster species as minor components (1.0% each).

In addition to mixtures consisting of DNA from bivalve species only, a DNA extract mixture containing another mollusc species was prepared. DNA extract from a squid species (Sepiella inermis) was chosen as the main component (97.0%) and DNA from the bivalve species Placopecten magellanicus, Ostrea edulis and Perna canaliculus were present as minor components (1.0% each).

### Reference Sequences

A 150 bp fragment of the mitochondrial 16S rDNA gene was used as DNA barcode. Reference sequences for commonly consumed bivalve species and some exotic seafood species, that are permitted for consumption in Austria (“Codex Alimentarius Austriacus” chapter B35, (see Bundesministerium für Arbeit, Soziales, Gesundheit und Konsumentenschutz, Codexkapitel/B 35/Fische, Krebse, Weichtiere und daraus hergestellte Erzeugnisse: BMGF-75210/0026-II/B/13/2017, 2007), were downloaded from the NCBI databases by using CLC Genomics Workbench 10.1.1 (Qiagen). If available, complete reference sequences from the RefSeq database were preferentially downloaded due to their reliability. In case complete reference sequences were not available, all DNA sequences of the mitochondrial 16S rDNA available for one and the same species, submitted by individual scientists, were aligned and checked for similarity and unidentified nucleotides. Subsequently, the DNA sequence with the highest quality (e.g. without unknown nucleotides, full-length of the DNA barcode) was chosen as a reference sequence.

The reference sequences according to the invention are given in Table 4. This list of sequences comprises all reference sequences used for the identification of all seafood species of the invention and thus, does not only comprise the sequences for example 1 but for the seafood species identified according to the invention.

TABLE 4

| Acces- | | | SEQ |
|---|---|---|---|
| sion | | | ID |
| No. | Species | Sequence (nucleotide) | NO |
| NC_03 | Mytilus | CCTTTTGCATAAGGGTTTTTCAAGATCAATTTAAGAATTT | 28 |
| 0633 | chilensis | AATTTTCCCGAATAAAAGAGAGTTATTTTTTGGAGTTAGG | |
| | | AAATCGTGGTAGAGATTTGATTAAACTAAAGAATGGCGGC | |
| | | TAGATACTATTCG | |
| NC_02 | Perna | CCTTTTGCATAAGGGTTTTTCAAGAAAATTTACTTATGTA | 29 |
| 6288 | perna | TTTTCCCGAAAAGGAAGAAGCTATCGTTGTGAGTGTGAAT | |
| | | ACCGTGGTAAAGTTATGACCAACTATACGATAAAAGCTAG | |
| | | ATACTAATTGCCT | |
| NC_02 | Mytilus | CCTTTTGCATAAGGGTTTTTCAAGATAAATTTAAGCATTT | 30 |
| 4733 | unguiculatus | AAATTTCCCGAATAAAAGAGAGTTATTCTTTTGAGTTAAA | |
| | | AAATCGTGGTAGAGATTAATATAAACTAAAGAATGGCGGC | |
| | | TAGATACTATTCG | |
| NC_01 | Perna | CCTTTTGTATAAGGGTTTTTTTAGAAAATGAGTGTATAAC | 31 |
| 8362 | viridis | TGTTTCCCGAAAAAAGATAAGTTACTTGAATTTAATTTAT | |
| | | TTTTCGTGGTAAAGAAAAATAAAAAAATCAAGTAAAAGTG | |
| | | ATATGCTATTTG | |
| NC_01 | Mytilus | CCTTTTGCATAAGGGTTTTTCAAGATAAATTTAAATATTT | 32 |
| 5993 | californianus | AAATTTCCCGAATAAAAAAGAGTTATTCCTTTGAGTTAAA | |
| | | AAATCGTGGTAGAGATTTATATAAACTAAGGAATAGCGGC | |
| | | TAGATACTATTCG | |
| NC_00 | Mytilus | CCTTTTGCATAAGGGTTTTTCAAGACAAGTTTAAGAATTT | 33 |
| 7687 | trossulus | AATTTTCCCGAATAAAAGAGAGTTATTTTTTTGAGTTAAG | |
| | | AAATCGTGGCAGAGATTTAATTAAACTAAAGAATAGCGGC | |
| | | TAGATACTATTCG | |
| NC_00 | Mytilus | CCTTTTGCATAAGGGTTTTTCAAGATAAATTTAAGAATTT | 34 |
| 6886 | galloprovincialis | AATTTTCCCGAATAAAAGAGAGTTATTTTTTTGAGTTAGG | |
| | | AAATCGTGGTAGAGATTTGATTAAACTAAAGAATGGCGGC | |
| | | TAGATACTATTCG | |
| NC_00 | Mytilusedulis | CCTTTTGCATAAGGGTTTTTCAAGATCAATTTAAGAATTT | 35 |
| 6161 | | AATTTTCCCGAATAAAAGAGAGTTATTTTTTTGAGTTAGG | |
| | | AAATCGTGGTAGAGATTTGATTAAACTAAAGAATGGCGGC | |
| | | TAGATACTATTCG | |
| NC_05 | Perna | CCTTTTGTATAAGGGTTTTTCAAGAAACAAAACGTATGTT | 36 |
| 4242 | canaliculus | TGTATCCCGAAAAAAATAAAAGTTACTTTTTTTCTTATAT | |
| | | TTAACGTGGCAAAGTTAAATTATAAAAAGAGGTGAAAGTG | |
| | | ATATGCTATTTGC | |
| KP100 | Mytilus | CCTTTTGCATAAGGGTTTTTCAAGATCAATTTAAGAATTT | 37 |
| 301 | platensis | AATTTTCCCGAATAAAAGAGAGTTATTTTTTGGAGTTAGG | |
| | | AAATCGTGGTAGAGATTTGATTAAACTAAAGAATGGCGGC | |
| | | TAGATACTATTCG | |
| NC_01 | Magallana | GGTAGCGAAATTCCTTGCCTTTTGATTGTAGGCCTGCATG | 38 |
| 3997 | bilineata | AATGGTTTAACGAGGGTTTAGCTGTCTCTAAATTTTTAAT | |
| | | TGAAATTGTATTGAAGGTGAAGATACCTTCATTTAAAAGT | |
| | | TAGACAAAAAGACCCCGTGCAACTTT | |
| NC_00 | Magallana | GGTAGCGAAATTCCTTGCCTTTTGATTGTGGGCCTGCATG | 39 |
| 1276 | gigas | AATGGTTTAACGAGGGTTTGACTGTCTCTAAATTTTTTAT | |
| | | TGAAATTGTACTGAAGGTGAAGATACCTTCATTTAAAAGT | |
| | | TAGACAAAAAGACCCCGTGCAACTTT | |
| NC_00 | Crassostrea | GGTAGCGAAATTCCTTGCCTTTTGATTGTGGGCCTGCATG | 40 |
| 7175 | virginica | AATGGTTTGACGAGGGCTTTGCTGTCTCTTGATTTTTTAT | |
| | | TGAAATTGTAGTGTAGGTGAAAATACCTTCATAAGAAAGT | |
| | | TAGACAAGAAGACCCCGTGCAACTTT | |
| NC_01 | Magallana | GGTAGCGAAATTCCTTGCCTTTTGATTGTAGGCCTGCATG | 41 |
| 1518 | hongkongensis | AATGGTTTAACGAGGGTTTAACTGTCTCTAAATTTTTTAT | |
| | | TGAAATTGTACTGAAGGTGAAGATACCTTCATTTAAAAGT | |
| | | TAGACAAAAAGACCCCGTGCAACTTT | |
| NC_01 | Magallana | GGTAGCGAAATTCCTTGCCTTTTGATTGTGGGCCTGCATG | 42 |
| 2648 | angulata | AATGGTTTAACGAGGGTTTAACTGTCTCTAAATTTTTTAT | |
| | | TGAAATTGTACTGAAGGTGAAGATACCTTCATTTAAAAGT | |
| | | TAGACAAAAAGACCCCGTGCAACTTT | |
| NC_01 | Magallan | GGTAGCGAAATTCCTTGCCTTTTGATTGTAGGCCTGCATG | 43 |
| 2649 | asikamea | AATGGTTTAACGAGGGTTTAACTGTCTCTAAATTTTTTAT | |
| | | TGAAATTGTACTGAAGGTGAAGATACCTTCATTAAAAAGT | |
| | | TAGACAAAAAGACCCCGTGCAACTTT | |
| NC_01 | Magallana | GGTAGCGAAATTCCTTGCCTTTTGATTGTAGGCCTGCATG | 44 |
| 2650 | ariakensis | AATGGTTTAACGAGGGTTTAACTGTCTCTTGATTTAAAAT | |
| | | TGAAATTGTACTGAAGGTGAAGATACCTTCATTAAAAAGT | |
| | | CAGACAAAAAGACCCCGTGCAACTTT | |
| NC_01 | Ostrea | GGTAGCGAAATTCCTTGCCTTTTAATTGTAGGCCTGCATG | 45 |
| 5231 | denselamellosa | AATGGCTTAACGAGGGCTTAACTGTCTCTGGTTTACAGGG | |
| | | TCTAAATTGGATTAAAGGTGAAGATACCTTTATATAAAAG | |
| | | TCAGACAAGAAGACCCCGTGCAACTTT | |
| NC_01 | Magallana | GGTAGCGAAATTCCTTGCCTTTTGATTGTGGGCCTGCATG | 46 |
| 5248 | nippona | AATGGTTTAACGAGGGTTTGACTGTCTCTAAGTTTTTTAT | |
| | | TGAAATTGTATTGAAGGTGAAGATACCTTCATTTAAAAGT | |
| | | TAGACAAAAAGACCCCGTGCAACTTT | |
| NC_01 | Ostreaedulis | GGTAGCGAAATTCCTTGCCTTTTAATTGTAGGCCTGCATG | 47 |
| 6180 | | AATGGTTTGACGAGGGCTTAACTGTCTCTAGTTTGTAAGT | |
| | | CTAAATTGGATTAAAGGTGAAGATACCTTTATAAAAAAGT | |
| | | CAGACAAGAAGACCCCGTGCAACTTT | |
| NC_02 | Ostrea | GGTAGCGAAATTCCTTGCCTTTTAATTGTAGGCCTGCATG | 48 |
| 2688 | lurida | AATGGCTTGACGAGGGTTTAACTGTCTCTGGTTTGTAAGG | |
| | | TCTAAATTGGATTAAAGGTGAAGATACCTTTATATAAAAG | |
| | | TCAGACAAGAAGACCCCGTGCAACTTT | |
| NC_02 | Crassostrea | GGTAGCGAAATTCCTTGCCTTTTAATTGTAGGCCAGCATG | 49 |
| 7653 | tulipa | AATGGTTTGACGAGGGCCTCACTGTCTCTTAGTTCTATGT | |
| | | TGAAATTGTAGTGTAGGTGAAGATACCTTCATAAAAAAGT | |
| | | AAGACAAAAAGACCCCGTGCAACTTT | |
| AF052 | Ostrea | GGTAGCGAAATTCCTTGCCTTTTAATTGTAGGCCTGCATG | 50 |
| 063 | angasi | AATGGTTTGACGAGGGCTTAACTGTCTCTAGTTTGTAAGT | |
| | | CTAAATTGGACTAAAGGTGAAGATACCTTTATAAAAAAGT | |
| | | CAGACAAGAAGACCCCGTGCAACTTT | |
| NC_03 | Magallana | GGTAGCGAAATTCCTTGCCTTTTGATTGTAGGCCTGCATG | 51 |
| 7851 | belcheri | AATGGTTTAACGAGGGTTTAACTGTCTCTAAATTTTTAGT | |
| | | TGAAATTGTATTGAAGGTGAAGATACCTTCATTAAGAAGT | |
| | | TAGACAAAAAGACCCCGTGCAACTTT | |
| FJ717 | Crassostrea | GGTAGCGAAATTCCTTGCCTTTTGATTGTGGGCCAGCATG | 52 |
| 607 | rhizophorae | AATGGTTTGACGAGGGCTTTGCTGTCTCTCGATTTTTTAT | |
| | | TGAAATTGTAGTGTAGGTGAAAATACCTTCATAAGAAAGT | |
| | | TAGACAAGAAGACCCCGTGCAACTTT | |
| HQ711 | Crassostrea | GGTAGCGAAATTCCTTGCCTTTTAATTGTAGGCCAGCATG | 53 |
| 627 | brasiliana | AATGGTTTGACGAGGGCCTCACTGTCTCTTAGTTCTATGT | |
| | | TGAAATTGTAGTGTAGGTGAAGATACCTTCATAAAAAAGT | |
| | | AAGACAAAAAGACCCCGTGCAACTTT | |
| KX364 | Talonostrea | GGTAGCGAAATTCCTTGCCTTTTAATTGTAGGCCTGCATG | 54 |
| 275 | talonata | AATGGTTTAACGAGGGTTAAGCTGTCTCTAAGTTTATAAG | |
| | | TTGAAATTGTATTAAAGGTGAAGATACCTTTATTTTGAAG | |
| | | TTAGACAAAAAGACCCCGTGCAACTTT | |
| KT317 | Crassostrea | GGTAGCGAAATTCCTTGCCTTTTAATTGTAGGCCAGCATG | 55 |
| 088 | corteziensis | AATGGTTTGACGAGGGCCTCACTGTCTCTCAGCTCTATAT | |
| | | TGAAATTGCAGTGGAGGTGAAGATACCTCCATAAGAAAGT | |
| | | AAGACAAGAAGACCCCGTGCAACTTT | |
| AY510 | Magallana | GGTAGCGAAATTCCTTGCCTTTTGATTGTAGGCCTGCATG | 56 |
| 450 | rivularis | AATGGTTTAACGAGGGTTTAACTGTCTCTAAATTTTTTAT | |
| | | TGAAATTGTACTGAAGGTGAAGATACCTTCATTTAAAAGT | |
| | | TAGACAAAAAGACCCCGTGCAACTTT | |
| KT317 | Ostrea | GGTAGCGAAATTCCTTGCCTTTTAATTGTAGGCCTGCATG | 57 |
| 130 | angelica | AATGGCTTGACGAGGGTTTAACTGTCTCTGGTTTGTAAGG | |
| | | TCTAAATTGGATTAAAGGTGAAGATACCTTTATATAAAAG | |
| | | TCAGACAAGAAGACCCCGTGCAACTTT | |
| AF052 | Ostrea | GGTAGCGAAATTCCTTGCCTTTTAATTGTAGGCCTGCATG | 58 |
| 075 | permollis | AATGGCTTGACGAGGGTTTAACTGTCTCTGGTTTGTAAAG | |
| | | TCTAAATTGGATTAAAGGTGAAGATACCTTTATATAAAAG | |
| | | TCAGACAAGAAGACCCCGTGCAACTTT | |
| JF808 | Ostrea | GGTAGCGAAATTCCTTGCCTTTTAATTGTAGGCCTGCATG | 59 |
| 186 | chilensis | AATGGCTTGACGAGGGTTTAACTGTCTCTGGTTTATAAAG | |
| | | TCTAAATTGGATTAAAGGTGAAGATACCTTTATACAAAAG | |
| | | TCAGACAAGAAGACCCCGTGCAACTTT | |
| AF052 | Ostrea | GGTAGCGAAATTCCTTGCCTTTTAATTGTAGGCCTGCATG | 60 |
| 062 | algoensis | AATGGCTTGACGAGGGCTTAACTGTCTCTAGTTTTAAAGT | |
| | | CTAAATTGGATTAAAGGTGAAGATACCTTTATAAAAAAGT | |
| | | CAGACAAGAAGACCCCGTGCAACTTT | |
| AF052 | Ostrea | GGTAGCGAAATTCCTTGCCTTTTAATTGTAGGCCTGCATG | 61 |
| 073 | puelchana | AATGGCTTGACGAGGGTTTAACTGTCTCTGGTTTGTAAAG | |
| | | TCTAAATTGGATTAAAGGTGAAGATACCTTTATATAAAAG | |
| | | TCAGACAAGAAGACCCCGTGCAACTTT | |
| KX364 | Ostrea | GGTAGCGAAATTCCTTGCCTTTTAATTGTAGGCCTGCATG | 62 |
| 274 | megodon | AATGGCTTGACGAGGGTTTAACTGTCTCTGGTTTGTAAGG | |
| | | TCTAAATTGGATTAAAGGTGAAGATACCTTTATATGAAAG | |
| | | TCAGACAAGAAGACCCCGTGCAACTTT | |
| NC_02 | Saccostrea | GGTAGCGAAATTCCTTGCCTTTTAATTGTAGGCCTGCATG | 63 |
| 7724 | cuccullata | AATGGATTGACGAGGGTCAAGCTGTCTCTTGCCTTGAATA | |
| | | ACGAAATTGGACTAAAGGTGAAGATACCTTTATAGAAAAG | |
| | | TTAGACAAGAAGACCCCGTGCAGCTTT | |
| KT317 | Saccostrea | GGTAGCGAAATTCCTTGCCTTTTAATTGTAGGCCTGCATG | 64 |
| 278 | palmula | AATGGATTGACGAGGGTCAAACTGTCTCTTGCTTTGTATA | |
| | | GCGAAATTGGACTAAAGGTGAAGATACCTTTATATAAAAG | |
| | | TTAGACAAGAAGACCCCGTGCAGCTTT | |
| KX961 | Saccostrea | GGTAGCGAAATTCCTTGCCTTTTAATTGTAGGCCTGCATG | 65 |
| 677 | malabonensis | AATGGATTGACGAGGGTCAAGCTGTCTCTTGCCTTGATGA | |
| | | CGAAATTGGACTAAAGGTGAAGATACCTTTATAGAAAAGT | |
| | | TAGACAAGAAGACCCCGTGCAGCTTT | |
| NC_01 | Saccostre | GGTAGCGAAATTCCTTGCCTTTTAATTGTAGGCCTGCATG | 66 |
| 3998 | ascyphophilla | AATGGATTGACGAGGGCCATTCTGTCTCTTGCCTTATGAA | |
| | | GTGAAATTGGATTAAAGGTGAAGATACCTTTATTAAAAAG | |
| | | TTAGACAAGAAGACCCCGTGCAGCTTT | |
| NC_03 | Saccostrea | GGTAGCGAAATTCCTTGCCTTTTAATTGTAGGCCTGCATG | 67 |
| 6483 | glomerata | AATGGATTGACGAGGGTCAAACTGTCTCTTGCTTTGTATG | |
| | | ACGAAATTGGACTGAAGGTGAAGATACCTTCATGTGAAAG | |
| | | TTAGACAAGAAGACCCCGTGCAGCTTT | |
| NC_03 | Saccostrea | GGTAGCGAAATTCCTTGCCTTTTAATTGTAGGCCTGCATG | 68 |
| 0533 | kegaki | AATGGATTGACGAGGGTCAAACTGTCTCTTGCTTTGTATG | |
| | | TCGAAATTGGACTGAAGGTGAAGATACCTTCATATAAAAG | |
| | | TTAGACAAGAAGACCCCGTGCAGCTTT | |
| NC_03 | Saccostre | GGTAGCGAAATTCCTTGCCTTTTAATTGTAGGCCTGCATG | 69 |
| 6478 | aechinata | AATGGATTGACGAGGGTCAAACTGTCTCTTGCTTTGTATG | |
| | | ACGAAATTGGACTGAAGGTGAAGATACCTTCATGTGAAAG | |
| | | TTAGACAAGAAGACCCCGTGCAGCTTT | |
| NC_03 | Saccostrea | GGTAGCGAAATTCCTTGCCTTTTAATTGTAGGCCTGCATG | 70 |
| 6479 | mytiloides | AATGGATTGACGAGGGTCAAACTGTCTCTTGCTTTGTATG | |
| | | ACGAAATTGGACTGAAGGTGAAGATACCTTCATGTGAAAG | |
| | | TTAGACAAGAAGACCCCGTGCAGCTTT | |
| AJ972 | Euvola | TGCTAAGGTAGCTAAATTATGGCCTATTAATTGTAGGTCC | 71 |
| 431 | vogdesi | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| NC_01 | Mimachlamys | TGCTAAGGTAGCTAAATTATGGCCCATTAATTGTGGGTCT | 72 |
| 1608 | crassicostata | TGCGAACGGCTTGACGAGTTCCCTACTGTCTCAAAGTTGT | |
| | | TTTGGTGAACTTGAATTGGATGTGTAAATGCTTCCATGAG | |
| | | TGAGAAAGACGAGAAGACCCCGTGAAG | |
| EU379 | Gloripallium | TGCTAAGGTAGCTAAATTACGGCCTATTAATTGTAGGTCC | 73 |
| 464 | pallium | TGTGAATGGTTTGACGAGCCTCCAACTGTCTCTAGTTTAT | |
| | | TTTGGTGAACTTGAATTGGATGTGCAAATGCTTCCATAGG | |
| | | TAAGAAAGACGAGAAGACCCCGTGAAG | |
| AJ243 | Flexopecten | TGCTAAGGTAGCTAAGTTATGGTCTTTTAATTTTAGGCCC | 74 |
| 574 | glaber | TGTGAAAGGATTGACGAGCTTGAGTCTGTCTCTATCTAAA | |
| | | TTTTATGAAATTGAATTTTAAGTGCAAATGCTTAAGTGGA | |
| | | CATGAAAGACGAGAAGACCCCGTGAAG | |
| AJ245 | Pecten | TGCTAAGGTAGCTAAATTATGGCCTATTAATTGTAGGTCC | 75 |
| 394 | jacobaeus | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| AY650 | Pecten | TGCTAAGGTAGCTAAATTATGGCCTATTAATTGTAGGTCC | 76 |
| 055 | novaezelandiae | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| EU379 | Euvola | TGCTAAGGTAGCTAAATTATGGCCTATTAATTGTAGGTCC | 77 |
| 473 | raveneli | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| AM494 | Aequipecten | TGCTAAGGTAGCTAAATTATGGCCCTCTAATTGGGGGCCC | 78 |
| 408 | opercularis | TGTGAATGGATTGACGAGCCTATAACTGTCTCTAGCTGAA | |
| | | TTATATGAAATTGAATTTTAAGTGCAAATGCTTAAATATA | |
| | | AGTGATAGACGAGAAGACCCCGTGAAG | |
| HM630 | Euvola | TGCTAAGGTAGCTAAATTATGGCCTATTAATTGTAGGTCC | 79 |
| 517 | perula | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| GQ342 | Nodipecten | TGCTAAGGTAGCTAAATTATGGCCTATTAATTGTAGGTCC | 80 |
| 275 | nodosus | TGTGAATGGTTTGACGAGTCTTCAACTGTCTCTAGTTTTT | |
| | | TTGATGAACTTGAATTGGATGTGCAAATGCTTCCATGGGT | |
| | | AAGAAAGACGAGAAGACCCCGTGAAG | |
| GQ166 | Scaeochlamys | TGCTAAGGTAGCTAAATTATGGCCCATTAATTGTGGGTCC | 81 |
| 559 | livida | TGTGAATGGTTTGACGAGTCCTCGACTGTCTCAAGGTTGT | |
| | | TTTGGTGAACTTGAATTGAATGTGCAAATGCTTTCATGGG | |
| | | TAAGAAAGACGAGAAGACCCCGTGAAG | |
| FN667 | Pecten | TGCTAAGGTAGCTAAATTATGGCCTATTAATTGTAGGTCC | 82 |
| 667 | keppelianus | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| FN667 | Talochlamys | TGCTAAGGTAGCTAAATTATGGCCTATTAATTGTAGGTCC | 83 |
| 665 | multistriata | TGTGAATGGTTTGACGAGTCCTCTACTGTCTCAAGGTTGT | |
| | | TTTGGTGAACTTGAATGGAACGTGCAAATGCTTTCATGAG | |
| | | AGAGAAAGACGAGAAGACCCCGTGAAG | |
| FJ263 | Patinopecten | TGCTAAGGTAGCTAAATTATGGCCCATTAATTGTGGGTCC | 84 |
| 642 | caurinus | TGTGAATGGTTTGACGAGTCCTCTACTGTCTCAAGGTTGT | |
| | | TTTGGTGAACTTGAATTGAACGTGCAAATGCTTTCATGGG | |
| | | AAAGAAAGACGAGAAGACCCCGTGAAG | |
| FJ263 | Chlamys | TGCTAAGGTAGCTAAATTATGGCCCATTAATTGTGGGTCC | 85 |
| 641 | behringiana | TGTGAATGGTTTGACGAGTCCTCGACTGTCTCAAGGTTGT | |
| | | TTTGGTGAACTTGAATTGAATGTGCAAATGCTTTCATGAG | |
| | | AAAGAAAGACGAGAAGACCCCGTGAAG | |
| EU379 | Placopecten | TGCTAAGGTAGCTAAATTATGGTCTATTAATTGTAGGTCT | 86 |
| 475 | septemradiatus | TGTGAATGGTTTGACGAGTCTTCGACTGTCTCTAGGTTGT | |
| | | TTTGGTGAACTTGAATTGGATGTGTAAATGCTTCCGTGGG | |
| | | TGAGAAAGACGAGAAGACCCCGTGAAG | |
| KP900 | Pecten | TGCTAAGGTAGCTAAATTATGGCCTATTAATTGTAGGCCC | 87 |
| 975 | maximus | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| KP900 | Pecten | TGCTAAGGTAGCTAAATTATGGCCTATTAATTGTAGGTCC | 88 |
| 974 | albicans | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| KP300 | Zygochlamys | TGCTAAGGTAGCTAAATTATGGCCCATTAATTGTGGGTCC | 89 |
| 542 | delicatula | TGTGAATGGTTTGACGAGTCCTCGACTGTCTCGAGATTAC | |
| | | TTTGGTGAACTTGAATTAAACGTGCAAATGCTTTTATGGG | |
| | | TAAGAAAGACGAGAAGACCCCGTGAAG | |
| KF982 | Chlamys | TGCTAAGGTAGCTAAATTATGGCCCATTAATTGTGGGTCC | 90 |
| 789 | hastata | TGTGAATGGTTTGACGAGTCCTCGACTGTCTCAAGGTTGT | |
| | | TTTGGTGAACTTGAATTGAACGTGCAAATGCTTTCATGAG | |
| | | AAAGAAAGACGAGAAGACCCCGTGAAG | |
| KF982 | Ylistrum | TGCTAAGGTAGCTAAATTATGGCCTATTAATTGTAGGTCC | 91 |
| 785 | japonicum | TGTGAATGGTTTGACGAGTCTTTAACTGTCTCTAGTTTGT | |
| | | TTTGGTGAAATTGAATTGGATGTGCAAATGCTTCCGTAGG | |
| | | TAAGAAAGACGAGAAGACCCCGTGAAG | |
| JF339 | Pecten | TGCTAAGGTAGCTAAATTATGGCCTATTAATTGTAGGTCC | 92 |
| 109 | fumatus | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| JF339 | Talochlamys | TGCTAAGGTAGCTAAATTATGGCCCATTAATTGTGGGTCC | 93 |
| 106 | gemmulata | TGTGAATGGTTTGACGAGTCCTCGACTGTCTCAAGATTAC | |
| | | TTTGGTGAACTTGAATTAAATGTGCAAATGCTTTTATGGG | |
| | | TAAGAAAGACGAGAAGACCCCGTGAAG | |
| HM630 | Zygochlamys | TGCTAAGGTAGCTAAATTATGGCCCATTAATTGTGGGTCC | 94 |
| 521 | patagonica | TGTGAATGGTTTGACGAGTCCTCGACTGTCTCGAGATTAC | |
| | | TTTGGTGAACTTGAATTAAATGTGCAAATGCTTTTATGGG | |
| | | TAAGAAAGACGAGAAGACCCCGTGAAG | |
| NC_02 | Argopecten | TGCTAAGGTAGCTAAGTTATGGCCTAGTTAATTGTAGGCC | 95 |
| 7943 | purpuratus | CTGTGAATGGTTTGACGAGTTTTCTTCTGTCTCTAGCTTG | |
| | | TTTCAGTGAACTTGAATTGGATGTGCAAATGCTTCCTTGC | |
| | | GAAAGAAAGACGAGAAGACCCCGTGAAG | |
| NC_01 | Argopecten | TGCTAAGGTAGCTAAGTTATGGCCTAGTTAATTGTAGGCC | 96 |
| 2977 | irradians | CTGTGAATGGTTCGACGAGTTTTCTTCTGTCTCTAGCTTG | |
| | | TTTAAGTGAACTTGAATTGGATGTGCAAATGCTTCCTTGG | |
| | | TAAAGAAAGACGAGAAGACCCCGTGAAG | |
| NC_01 | Azumapecten | TGCTAAGGTAGCTAAATTATGGCCTATTAATTGTAGGTCC | 97 |
| 2138 | farreri | TGTGAATGGTTTGACGAGTCCTCGACTGTCTCGAGGTTGT | |
| | | TTTGGTGAACTTGAATTGAATGTGCAAATGCTTTCATGGG | |
| | | AAAGAAAGACGAGAAGACCCCGTGAAG | |
| NC_00 | Mizuhopecten | TGCTAAGGTAGCTAAATTATGGCCCATTAATTGTGGGTCC | 98 |
| 9081 | yessoensi | TGTGAATGGTTTGACGAGTCCTCTACTGTCTCAAGGTTGT | |
| | | TTTGGTGAACTTGAATTGAATGTGCAAATGCTTTCATACG | |
| | | AAAGAAAGACGAAGAAGACCCCGTGAAG | |
| NC_00 | Placopecten | TGCTAAGGTAGCTAAGTTATGGCCTATTAATTGTAGGTCT | 99 |
| 7234 | magellanicus | TGTGAATGGTTTGACGAGCCTCCAACTGTCTCTAGGTTGT | |
| | | TTTGGTGAACTTGAGTTGAATGTGTAAATGCCTTCGTGGG | |
| | | TAAGAAAGACGAGAAGACCCCGTGAAG | |
| EU379 | Euvola | TGCTAAGGTAGCTAAATTATGGCCTATTAATTGTAGGTCC | 100 |
| 485 | ziczac | TGTGAATGGTTTGACGAGTTTCCAACTGTCTCTAGTTTTT | |
| | | TTTGGTGAACTTGAATTGGATGTGCAAATGCTTCCATGGG | |
| | | TAAGAAAGACGAGAAGACCCCGTGAAG | |
| KU754 | Pecten | TGCTAAGGTAGCTAAATTATGGCCTATTAATTGTAGGTCC | 101 |
| 458 | sulcico | TGTGAATGGTTTGACGAGTTTCCAACTGTCTCTAGTTTTT | |
| | status | TTTGGTGAACTTGAATTGGATGTGCAAATGCTTCCATGGG | |
| | | TAAGAAAGACGAGAAGACCCCGTGAAG | |
| KR827 | Chlamys | TGCTAAGGTAGCTAAATTATGGCCCATTAATTGTGGGTCC | 102 |
| 548 | islandica | TGTGAATGGTTTGACGAGTCCTCGACTGTCTCAAGGTTGT | |
| | | TTTGGTGAACTTGAATTGAATGTGTAAATGCTTTCATGAG | |
| | | AAAGAAAGACGAGAAGACCCCGTGAAG | |
| KT161 | Argopecten | TGCTAAGGTAGCTAAGTTATGGCCTAGTTAATTGTAGGCC | 103 |
| 261 | ventricosus | CTGTGAATGGTTTGACGAGTTTTCCTCTGTCTCTAGCTTG | |
| | | TTTTAGTGAACTTGAATTGGATGTGCAAATGCTTCCTTGA | |
| | | GAAAGAAAGACGAGAAGACCCCGTGAAG | |
| KT988 | Mimachlamys | TGCTAAGGTAGCTAAATTATGGCCTATTAATTGTAGGTCC | 104 |
| 340 | varia | TGTGAATGGTTTGACGAGTCCCCTACTGTCTCGAGGTTGT | |
| | | TTTGGTGAAATTGAATTGAACGTGCAAATGCTTTCATGGG | |
| | | GAAGAAAGACGAGAAGACCCCGTGAAG | |
| KP900 | Amusium | TGCTAAGGTAGCTAAATTATGGCCTATTAATTGTAGGTCC | 105 |
| 978 | pleuronectes | TGTGAATGGTTTGACGAGTTTCCAACTGTCTCTAATTTTT | |
| | | TTTGGTGAACTTGAATTGGATGTGCAAATGCTTCCATGGG | |
| | | TAAGAAAGACGAGAAGACCCCGTGAAG | |
| NC_02 | Mimachla | TGCTAAGGTAGCTAAATTATGGCCCATTAATTGTGGGTCC | 106 |
| 2416 | myssanguinea | TGTGAATGGTTTGACGAGTCCTCGGCTGTCTCAAGGTTGT | |
| | | TTTGGTGAACTTGAATTGAATGTGCAAATGCTTTCATAGG | |
| | | TAAGAAAGACGAGAAGACCCCGTGAAG | |
| KP300 | Talochlamys | TGCAAAGGTAGCTAAATTATGGCCCATTAATTGTGGGTCC | 107 |
| 543 | dichroa | TGTGAACGGTTTGACGAGTCCTCGACTGTCTCAAGATTAC | |
| | | TTTGGTGAACTTGAATTAAATGTGCAAATGCTTTTATGGG | |
| | | TAAGAAAGACGAGAAGACCCCGTGAAG | |
| KP300 | Mimachla | TGCTAAGGTAGCTAAATTATGGCCCATTAATTGTGGGTCT | 108 |
| 549 | mys | TGCGAACGGCTTGACGAGTTCCCTACTGTCTCAAAGTTGT | |
| | gloriosa | TTTGGTGAACTTGAATTGGATGTGCAAATGCTTCCATGAG | |
| | | TAAGAAAGACGAGAAGACCCCGTGAAG | |
| JF339 | Mimachlamys | TGCTAAGGTAGCTGAATTCTGGCCCATTAAATGTGGGTCT | 109 |
| 118 | cloacata | TAGTGAATGGTTTGACGAGTCCTCGACTGTCTCAAGGTTG | |
| | | TTTTTTTGAAATTGAATTAAATGTGCAAATGCATTTATAG | |
| | | GGAAGAAAGACGAGAAGACCCCGTGAAG | |
| JF339 | Mimachlamys | TGCTAAGGTAGCTAAATTTTGGCCCATTAATTGTGGGTCT | 110 |
| 117 | asperrima | TGTGAATGGTTTGACGAGTTCCCTACTGTCTCAAAGTTGT | |
| | | TTTGGTGAACTTGAATTGGATGTGCAAATGCATCTGTAGG | |
| | | TAAGAAAGACGAGAAGACCCCGTGAAG | |
| KF982 | Annachla | TGCTAAGGTAGCTAAATTATGGCCTATTAATTGTAGGTCC | 111 |
| 786 | mysstriatula | TGTGAATGGTTTGACGAGTCTTCAACTGTCTCTGATTTGC | |
| | | TTTGGTGAAATTGAATTGGAAGTGCAAATGCTTCCGTAGG | |
| | | TAAGAAAGACGAGAAGACCCCGTGAAG | |
| KF982 | Decatopecten | TGCAAAGGTAGCTAAATTATGGCCTATTAATTGTAGGTCC | 112 |
| 788 | radula | TGTGAATGGTTTGACGAGCTCCCGACTGTCTCTAGTTTGG | |
| | | TTTAGTGAACTTGAATTGGATGTGCAAATGCTTCCATGGG | |
| | | AAAGAAAGACGAGAAGACCCCGTGAAG | |
| KF982 | Bractechlamys | TGCAAAGGTAGCTAAATTACGGCCTATTAATTGTAGGTCC | 113 |
| 787 | vexillum | TGTGAATGGTTTGACGAGCCTCCGGCTGTCTCTAATCTGT | |
| | | TTTGGTGAACTTGAATTGGACGTGCAAATGCTTCCTTGAA | |
| | | AAAGAAAGACGAGAAGACCCCGTGAAG | |
| EU379 | Aequipecten | TGCTAAGGTAGCTAAATTATGGCCTATTAATTGTAGGTCC | 114 |
| 445 | glyptus | TGTGAATGGTTTGACGAGTTTTTAACTGTCTCTGGTTTGT | |
| | | TTTGGTGAATTTGAATTGGAAGTGCAAATGCTTTCGTGAG | |
| | | TGAGAAAGACGAGAAGACCCCGTGAAG | |
| KP300 | Scaeochlamys | TGCTAAGGTAGCTAAATTATGGCCTATTAATTGTAGGTCC | 115 |
| 554 | lemniscata | TGTGAATGGTTTGACGAGTCTTCAACTGTCTCAAAGTTAT | |
| | | TTTGGTGAACTTGAATTAAATGTGCAAATGCATTTATAGG | |
| | | GAAGAAAGACGAGAAGACCCCGTGAAG | |
| FJ263 | Chlamys | TGCTAAGGTAGCTAAATTATGGCCCATTAATTGTGGGTCC | 116 |
| 645 | rubida | TGTGAATGGTTTGACGAGTCCTCGACTGTCTCAAGGTTGT | |
| | | TTTGGTGAACTTGAATTGAATGTGCAAATGCTTTCATGAG | |
| | | AAAGAAAGACGAGAAGACCCCGTGAAG | |
| JF496 | Karneka | TGCTAAGGTAGCTAAATTTTGGTCTATTAATTGTAGGTCT | 117 |
| 755 | mpiasulcata | TGTGAATGGTTTGACGAGTCTTCGACTGTCTCTAGGTTGT | |
| | | TTTGGTGAACTTGAATTGAATGTGTAAATGCTTTCGTGGG | |
| | | CGAGAAAGACGAGAAGACCCCGTGAAG | |
| FJ263 | Crassadoma | TGCTAAGGTAGCTAAATTATGGCCCATTAATTGTGGGTCC | 118 |
| 644 | gigantea | TGTGAATGGTTTGACGAGTCCTCGACTGTCTCAAGGTTGT | |
| | | TTTGGTGAACTTGAATTGAACGTGCAAATGCTTTCATGGG | |
| | | AGAGAAAGACGAGAAGACCCCGTGAAG | |
| JF339 | Ylistrum | TGCTAAGGTAGCTAAATTATGGCCTATTAATTGTAGGTCC | 119 |
| 127 | balloti | TGTGAATGGTTTGACGAGTCTTTAACTGTCTCTAGTTTGT | |
| | | TTTGGTGAAATTGAATTGGATGTGCAAATGCTTCCATAGG | |
| | | TGAGAAAGACGAGAAGACCCCGTGAAG | |
| AF122 | Tridacna | AGTAGCGTGATAAGTCGGCCTTTAATTGGGGTCTGGTATG | 120 |
| 977 | mbalavua | AAGGGGTTGACGTCGGAGAAGCTGTCTCAAAAGAATTTTG | |
| | na | TGAAATTTACTTTCTAGTGAAAAGGCTAGAATAATATTAA | |
| | | AAGACGAGAAGACCCTGTCGAGC | |
| AB751 | Siliqua | GGTAGCGTAATAAATTGTCTCTTAATTGGAGAAGGGTATG | 121 |
| 362 | alta | AATGGTTTGACGTAAGGGTTTCTGTCTCAACAATAGTAAT | |
| | | GTGAAGTTTTCTTTTAAGTCAAAAGGCTTAAATTATATTA | |
| | | AAAGACGAGAAGACCCTGTCGAGC | |
| AB751 | Megangulus | GGTAGCATAATAGGTCGTCCTTTAATTGGGGGAAAGTATG | 122 |
| 329 | zyonoensis | AATGGTCGGATGTTGGAAATTCTTTATTAAGAGTAGGTGT | |
| | | TAAATTTTACTTTTTAGTGCAAAGGCTAAGATAATATTAT | |
| | | TAGACGAGAAGACCCTGTTGAGC | |
| AB751 | Megangulus | GGTAGCATAATAGGTCGTCCTTTAATTGGGGGAAAGTATG | 123 |
| 328 | venulosus | AATGGTCGGATGTTGGAAATCCTTTATTAAGAGTAGGTGT | |
| | | TAAATTTTACTTTTTAGTGCAAAGGCTAAAATTAGATTAT | |
| | | TAGACGAGAAGACCCTGTTGAGC | |
| AB751 | Donax | GCGTTAAAGTAGCATAATAGGTCGTCTTTTAATTGGAGAA | 124 |
| 311 | faba | TGGAATGAATGGAGTGACGATGGAAAATCTTTATTAGAAA | |
| | | TATAAGTTGAAGTTTATTTTTAAGTGAAAAGGCTTAAATT | |
| | | AAGGTGTTAGACGAGAAGACCCTATTGAGC | |
| AB751 | Donax | GCGTTAAGGTAGCATAATAGCTCGTCTTTTAATTGGAGGA | 125 |
| 310 | cuneatus | TGGAATGAATGGAGTGACGATGGAAAGTCTTTATTAAAAA | |
| | | CACTGATCGAAGTTTACTTTTAAGTGAAAAGGCTTAAATG | |
| | | GAGTTGTTAGACGAGAAGACCCTGTTGAGC | |
| AB751 | Donax | GCGTTAAAGTAGCATAATAAATTGTCTCTTAATTAGAGGA | 126 |
| 309 | kiusiuensis | TAGTATGAAAGGTTGGATGTTGGAGAAGCTTTATTGGAAG | |
| | | CATTAATTGAAGTTTACATATAAGTGAAAAGGCTTATATG | |
| | | AGATTGTTAGACGAGAAGACCCTGTTGAGC | |
| DQ 160 | Mactra | GCAATAAATTGTCCTTTAATTGGGGAAATGAATGAAGGGG | 127 |
| 001 | quadrang | TTGACGAGGGGGAAGCTGTCTTTTTAATATAGTTCGAAGT | |
| | ularis | TTTCTTTTATGTGAAAAGACTTAGATTTTACAATAAGAAG | |
| | | AGAAGACCCCGGCGAGC | |
| AJ548 | Ensisensis | CTTAATTGGAGAAGGGTATGAATGGTTTGACGTGGTAGTT | 128 |
| 775 | | TCTGTCTCAATGGTAATATTATGAAGTTTTCTTTTGAGTT | |
| | | AAAAGTCTCAAATTTAGATAAAAGACGAGAAGACCCTGTC | |
| | | GAGC | |
| AM085 | Chamelea | GTGCTAAGGTAGCGTGATAAGTTGTCTTTTAATTGGAGAA | 129 |
| 110 | gallina | TGGTATGAATGGTTTAACGTAGAATAACTGTCTTTGGAAA | |
| | | ATAAAATTAAGTTTCCATGTAAGTGAAAAGGCTTATATTT | |
| | | TTGTAAAAGACGAGAAGACCCTGTCGAGC | |
| AJ548 | Spisula | GGTAGCGCAGTCACTCGTCTTTTAATTGGAGAAAGGTATG | 130 |
| 774 | subtruncata | AATGGTTTGACGAGGGAAAATCTGTCTCAAGGATATGGGC | |
| | | TAAAGTTTTCTTTTAAGTGAAAAGACTTAAATTTTTAAAT | |
| | | AAGAAGAGAAGACCCCGGCGAGC | |
| AJ548 | Callista | GTTTAACGGCCGCAGTTGTCCTGTGCTAAGGTAGCATAAT | 131 |
| 772 | chione | GATTAGCTCTTTAATTGGGGGAAAGTATGAATGGTTTGAC | |
| | | GTGGAGATACTGTCTTAAATATAATAAATAGAAATTTTTT | |
| | | TTCAAGTGAAAAAACCTGAATTTTTG | |
| AJ294 | Polititapes | TGTACTAAGGTAGCGCGGTAATTTGTCCCTTAATTGGGGG | 132 |
| 950 | rhomboides | ATGGTATGAAAGGCTTGTCTTAAGTTTTTTGTCTCAAAAA | |
| | | GACAAAGTGAAGTTTCCTTTTAGATGAAAAGGTCTAAATA | |
| | | AAAATAAAAGACGAGAAGACCCTGTCGAGC | |
| AJ417 | Venerupis | GGTAGCGCGATAATTTGTCTCTTAATTGGAGAATGGTATG | 133 |
| 845 | corrugata | AAAGGTTTGACGCAGATCAGTTGTCTCTTAGAAAAAAGGT | |
| | | GAAGTTTCCTTTTAAATGAGAAGGTTTAAATTTGAATAAA | |
| | | AGACGAGAAGACCCTGCCGAG | |
| AJ294 | Polititapes | TGTACTAAGGTAGCGCGATAATTTGTCTCTTAATTGGAGA | 134 |
| 950 | aureus | ATGGTATGAAAGGTTTGACGCAGATTGATTGTCTCTAGAA | |
| | | AAGAAAATGAAGTTTCCTTTTAAATGAAAAGGTTTAAATT | |
| | | GTAATAAAAGACGAGAAGACCCTGCCGAGC | |
| DQ459 | Venus | GTGCTAAGGTAGCGTGATAAGTTGTCCTTTGATTGGGGAA | 135 |
| 295 | crebrisulca | TGGTATGAAAGGTTTGACGTGGAATAACTGTCTTTAGAAA | |
| | | ATTAAACTAAGTTTCCATTTAAGTGAAAAGGCTTATATTT | |
| | | TTGTAAAAGACGAGAAGACCCTGTCGAGC | |
| DQ459 | Mercenaria | GGTAGCGTGATAAGTTGTCTTTTGATTGGAGAATGGAATG | 136 |
| 280 | campechiensis | AAGGGTTTGACGTAGAATTTCTTTTTTTGTAGAAGATACT | |
| | | AAAATTTCCTTCTAAGTGAAAAGGCTTAGGTTTTTGAAAA | |
| | | AGACGAGAAGACCCTGTCGAAC | |
| NC_05 | Antigona | GTGCTAAGGTAGCGTGATAGGTTGTCTCTTAATTGGAGAA | 137 |
| 1506 | lamellaris | TGGTATGAAGGGTCTCACGTGGGGTTTCTTTTTTTAATTA | |
| | | ATAAAATGAAGTTTCTTTTTAAGTGAAAAAGCTTAAGTTT | |
| | | TTGTAAAAGACGAGAAGACCCTGTCGAGC | |
| DQ459 | Ameghinomya | GTGCTAAGGTAGCGTGATAAGTTGTCCTTTGATTGGGGAC | 138 |
| 260 | antiqua | TGGTATGAATGGCTTGACGTAGAATGACTATTTTTATAGA | |
| | | ATAAAGTAAACTTTCCTTCTGAGTGAAAAGGCTTAGGTTT | |
| | | TTGTAAAAGACGAGAAGACCCTGTCGAGC | |
| DQ356 | Callistaerycina | GTTTAACGGCCGCAGTTGTCCTGTGCTAAGGTAGCATAAT | 139 |
| 382 | | AATTAGCTCTTTAATTGGGGGAAAGTATGAATGGTTTGAC | |
| | | GTGGAAATACTGTCTTTAATGTAATTAAGAGAAATTTTTT | |
| | | TATAGGTGAAAAAACCTAAATATTTGTAAGA | |
| DQ356 | Venerupis | GGTAGCGCGATAATTTGTCTCTTAATTGGAGAATGGTATG | 140 |
| 381 | aspera | AAAGGTTTGTCATAGAGCGGTTGTCTCTAAAAAATTAAGT | |
| | | GAAATTTCTTTTTAAATGAAAAGGTTTAAATAAAAATAAA | |
| | | AGACGAGAAGACCCTGTCGAGC | |
| DQ356 | Paphia | GGTAGCGTGATAATTTGTCCTTTAATTGGGGAATGGTATG | 141 |
| 380 | philippiana | AAAGGTTTGACGTGGATGAGTTGTCTCTAGAAAAATTATT | |
| | | GAAGTTTCCTTTTAAATGAAAAGGTTTAAATTTTTGTAAA | |
| | | AGACGAGAAGACCCTGGCGAG | |
| JF808 | Venus | GTGCTAAGGTAGCGTGGTAAGTTGTCTTTTAATTGGAGAA | 142 |
| 192 | casina | TGGTATGAATGGTTTGACGTAGAATAACTGTCTTTGGGGA | |
| | | ATAGAATTAAGTTTCCTTTTAAGTGAAAAGGCTTATATTT | |
| | | TTGTAAAAGACGAGAAGACCCTGTCGAGC | |
| HF970 | Ensis | CTTAATTGGAGAAGGGTATGAATGGTTTGACGTGGTAGTT | 143 |
| 514 | terranovensis | TCTGTCTCAATGGTAATATTATGAAGTTTTCTTTTGAGTT | |
| | | AAAAGTCTCAAATTTAGATAAAAGACGAGAAGACCCTGTC | |
| | | GAGC | |
| GQ166 | Mactra | GGTAGCGCAATCAATTGTCCTTTAATTGGGGAAAGGAATG | 144 |
| 567 | stultorum | AAGGGGTTGACGAGGGTGAGCTGTCTCTGTAGTATAGCTT | |
| | | AAACTTTTCTTTTAAGTGAAAAGACTTAAATTTTAAAATA | |
| | | AGACGAGAAGACCCCGGCGAGC | |
| HF970 | Ensis | CTTAATTGGAGAAGGGTATGAATGGTTTGACGTGGTAGTT | 145 |
| 473 | macha | TCTGTCTCAATGATAGTATTACGAAGTTTTCCTTTGAGTT | |
| | | AAAAGTCTCAAATAAATGTAAAAGACGAGAAGACCCTGTC | |
| | | GAGC | |
| EU169 | Siliqua | GGTAGCGTAATAAATTGTCTCTTAATTGGAGAAGGGTATG | 146 |
| 034 | minima | AATGGTTTGACGTAGGAGTTTCTGTCTCAACAATAGTAAT | |
| | | GCGAAGTTTTCCTTTAAGTAAAAAGGCTTAAATATTATTA | |
| | | AAAGACGAGAAGACCCTGTCGAGC | |
| GQ166 | Ensis | CTTAATTGGAGAAGGGTATGAATGGTTTGACGTGGTAGTT | 147 |
| 561 | leei | TCTGTCTCAATGGTAATATTATGAAGTTTTCTTTTGAGTT | |
| | | AAAAGTCTCAAATTTAGATAAAAGACGAGAAGACCCTGTC | |
| | | GAGC | |
| DQ459 | Polititapes | GGTAGCGCGATAATTTGTCTCTTAATTGGAGAATGGAATG | 148 |
| 296 | durus | AATGGTTTGTCGCATGTTGGTGTCTCTGGGAAAATGAAGT | |
| | | TTCCTTTTAATTGAAAAGGTTTAAATTTTAATAAAAGACG | |
| | | AGAAGACCCTGTGAGA | |
| KP052 | Cerastoderma | TTGGCCTTTAATTGGGGTCCGGTATGAATGGGTTGACGAT | 149 |
| 743 | glaucum | AGAAGCTCTGTCTCGTTAGAATGTGTTGAAATTTACATTT | |
| | | CGGTGAAAAGACCGAACCGCTTCTAAAAGACGAGAAGACC | |
| | | CCGTCGAG | |
| KF611 | Donax | GTACTAAGGTAGCGCTACAATTTGCCCTTTAAATGGGGGC | 150 |
| 752 | longissimus | TGGTATGAATGGGTTCACGTGAGAGTTGCTGTGTCACAAA | |
| | | AATTTATTGAAATTAACTTTTAGGTGAAGAGGCCTAGCTG | |
| | | AGTTTAAAGGACGACAAGACCCTAGAAGC | |
| KC429 | Solen | CTTAATTGGGGGCTAGTATGAAGGGTTTGACGTAAAAAAA | 151 |
| 308 | vaginoides | GTTGTCTCTGAGATAATATTGTGAAGTTTTTTTTCTGGTG | |
| | | AAAAATCTAGGATTTAATTAAAAGACGAGAAGACCCTGTC | |
| | | GAGC | |
| KC429 | Venus | GTGCTAAGGTAGCGTGATAAGTTGTCTTTTAATTGGAGAA | 152 |
| 301 | verrucosa | TGGTATGAATGGTTTGACGTAGAATAACTGTCTTTGGAAA | |
| | | ATTAAACTAAGTTTCCTTTTAAGTGAAAAGGCTTATATTT | |
| | | TTGTAAAAGACGAGAAGACCCTGTCGAGC | |
| JN969 | Ezocallista | ATTTAAAGGCTGCAGTTATCTGTACAAAGGTAGCGCAATA | 153 |
| 934 | brevisiphonata | AGTAGCCCCTTAATTGGGGGATGGTATGAATGGTTTGACG | |
| | | TGGAGATGCTGTTTCTAGTGTAGTTTAAATGAAATTTCTT | |
| | | TCTAGGTGAAAAAGCCTAGGTTTTTG | |
| KR422 | Procardium | AGTAGCGTAATAATTTGTCCCTTAATTAGGGTCTGTATGA | 154 |
| 892 | indicum | ACGGGTTGACGTGGAAAAACTGTCTTGAGGAAATAGTTTG | |
| | | AAATTTTCTTTTTAGTGAAAAGCCTGAAATAGTATTAAAA | |
| | | GACGAGAAGACCCCGTCGAGC | |
| KR422 | Cardium | AGTAGCGTAATAATTTGTCCCTTAATTAGGGTCTGTATGA | 155 |
| 893 | maxicostatum | AAGGGTTGACGTGGAAAAACTGTCTTGAAAAAATAATCTA | |
| | | AAATTTTCTTTTTAGTGAAAAGCCTGAAATGTGCTTAAAA | |
| | | GACGAGAAGACCCCGTCGAGC | |
| KR422 | Cardium | AGTAGCGTAATAATTTGTCCCTTAATTAGGGTCTGTATGA | 156 |
| 891 | costatum | AAGGGTTGACGTGGAAGAACTGTCTTGAAAAAATAATCTA | |
| | | AAATTTTCTTTTTAGTGAAAAGCCTAAGATATATTTAAAA | |
| | | GACGAGAAGACCCCGTCGAGC | |
| KR422 | Acanthocardia | TTGGCCTTTAATTGGGGTCCAGTATGAATGGGTTGACGAT | 157 |
| 879 | paucicostata | AGAATGGCTGTCTTATAGGGGGGCCTTAGAAATTTTCTTT | |
| | | TTGGTGAAAAGACCTAAATAAGTTTAATAGACGAGAAGAC | |
| | | CCCGTCGAG | |
| KR422 | Acanthoc | TTGGCCTTTAATTGGGGTCCAGTATGAATGGGTTGACGAT | 158 |
| 878 | ardiaechinata | AGAATAGCTGTCTTACAGGGGTTCATTAGAAATTTTCTTT | |
| | | TTGGTGAAAAGACCTGATTAAATTTAATAGACGAGAAGAC | |
| | | CCCGTCGAG | |
| KR422 | Acanthocardia | TTGGCCTTTAATTGGGGTCCAGTATGAATGGGTTGACGAT | 159 |
| 877 | aculeata | AGAATAGCTGTCTTATAGGGGCCCATTAGAAATTTTCTTT | |
| | | TTGGTGAAAAGACCTGATTAAACTTAATAGACGAGAAGAC | |
| | | CCCGTCGAG | |
| KP055 | Ruditapes | GGTAGCGCGATAATTTGTCTCTTAATTAGAGAATGGTATG | 160 |
| 816 | philippinarum | AAGGGTTAGACGTGGGATTATTGTCTCTAAAAAATACAGT | |
| | | TAAGTTTCCTTTTAAATGAAAAGGTTTAAATTTATTAAAA | |
| | | GACGAGAAGACCCTGTCGAGC | |
| KR422 | Corculum | AGTAGCATAATAATTTGCCCTTTAATTGGGGGATAGTATG | 161 |
| 901 | cardissa | AATGGGTTGACGATGGAAAGACTGTCTCCAGAAGAGTAAT | |
| | | TGAAATTTTCTTCCCAGTGAAAAGCCTGGGATTAGGTTGA | |
| | | AAGACGAGAAGACCCCGTCGAGC | |
| KX713 | Spisula | GGTAGCGCAATCAATCGTCTCTTAATTGGAGAAAGGTATG | 162 |
| 257 | solida | AATGGTTTGACGAGGGAAAAACTGTCTCAAATATAGATTT | |
| | | TGAAGTTTTCTTTTAAGTGAAAAGACTTAAATTTTTAAAT | |
| | | AAGAAGAGAAGACCCCGGTGAGC | |
| NC_04 | Scrobicularia | GGTAGCGCGATAGGTCGCTCTTTAATTGGGAGAGAGTATG | 163 |
| 6518 | plana | AATGGTCGGATGTTGGAAAGCCTTTATTAAAAGTAATTAT | |
| | | TGAAATTTACCTTTTAGTGCAAAGGCTAAAATGATTTTAT | |
| | | TAGACGAGAAGACCCTGTTGAGC | |
| KX713 | Mactra | GGTAGCGCAGTCAATTGTCCTTTAATTGGGGAAAGGAATG | 164 |
| 231 | violacea | AAAGGGTTGACGAGGGGGAGGCTGTCTTTGTAATATAATT | |
| | | TGAAATTTTCTTTTACGTGAAAAGACTTAAATTTTAAAAT | |
| | | AAGAAGAGAAGACCCCGGCGAGC | |
| KX713 | Chamelea | GTGCTAAGGTAGCGCGGTAAGTTGTCTTTTAATTGGAGAA | 165 |
| 203 | striatula | TGGTATGAATGGTTTAACGTAGAATAACTGTCTTTGAGGA | |
| | | ATAGAGTTAAATTTCCATTTAAGTGAAAAGGCTTATATTT | |
| | | TTGTAAAAGACGAGAAGACCCTATCGAAC | |
| KX713 | Ensissiliqua | CTTAATTGGAGAAGGGTATGAATGGTTTGACGTGGTAGTT | 166 |
| 217 | | TCTGTCTCAATAATAGTATTGCGAAATTTTCATTTAAGTT | |
| | | AAAAGTCTTAAATACAGATAAAAGACGAGAAGACCCTGTC | |
| | | GAGC | |
| KR422 | Serripes | AGTAGCGTAATAATTTGGCCCTTAATTAGGGTCTGGAATG | 167 |
| 986 | groenlandicus | AATGGGTTGACGTGGGAGGGCTGTCTCAAAGGGATTACTT | |
| | | AAAATTTTCTTCCTAGTGAAAAGCCTGGGATACTAGTAAA | |
| | | AGACGAGAAGACCCCGTCGAGC | |
| MN068 | Tridacna | AGTAGCGTGATAAGTCGGCCTTTAATTGGGGTCTGGTATG | 168 |
| 431 | elongatissima | AAGGGGTTGACGTCGGAAAAGCTGTCTCGGAAAAACTAAG | |
| | | TGAAATTTACTTTTAAGTGAAAAGGCTTAAATAAGGATAA | |
| | | AAGACGAGAAGGCCCTGTCGAGC | |
| MN068 | Tridacna | AGTAGCGTGATAAGTCGGCCTTTAATTGGGGTCTGGTATG | 169 |
| 543 | rosewateri | AAGGGGTTGACGTCGGAAAAGCTGTCTCAGAAAAACTAAG | |
| | | TGAAATTTACTTTTAAGTGAAAAGGCTTAAATGGGGGTAA | |
| | | AAGACGAGAAGGCCCTGTCGAGC | |
| NC_01 | Meretrix | TACCGCAGGGATAACAGCGTAATTTTTTTCCGTAAGTTGT | 170 |
| 6174 | lamarckii | ATTGGGAAAAAAGTTTACAACCTCGATGTTGAATTAAGAA | |
| | | AACTTTGTGGCGCAGACGCCACAGTGAGTAAAACTGTTCG | |
| | | TTTTTTAATCTCTTACATGA | |
| NC_01 | Meretrix | TACCGCAGGGATAACAGCGTAATTTTTTTTTGTAATTGTA | 171 |
| 4809 | lusoria | TCGATAAAAGAGTTTACGACCTCGATGTTGAATTAAGAAA | |
| | | CCTTTATGGCGCAGGAGCTATAGGAGGAAAACTGTTCGTT | |
| | | TTTTAATTTCTTACATGA | |
| NC_01 | Paphiaeuglypta | GGTAGCGCGATAATTTGTCTCTTAATTGGAGAATGGTATG | 172 |
| 4579 | | AAAGGTTTGACGTAGACCTATTGTCTCTAAAGAAAAAAAT | |
| | | GAAGTTTCCTTTTAAATGAAAAGGTTTAAATTTTTGTAAA | |
| | | AGACGAGAAGACCCTGCCGAGC | |
| NC_01 | Meretrix | TACCGCAGGGATAACAGCGTAATTTTTTTCTGTAATTGTA | 173 |
| 3188 | meretrix | TCGAGAAAAGAGTTTACGACCTCGATGTTGAATTAAGAAA | |
| | | CCTTTATGGCGCAGGAGCTATAGGAGGAAAACTGTTCGTT | |
| | | TTTTAATTTCTTACATGA | |
| NC_01 | Meretrix | TACCGCAGGGATAACAGCGTAATTTTTTTCTGTAATTGTA | 174 |
| 2767 | petechialis | TCGAGAAAAGAGTTTACGACCTCGATGTTGAATTAAGAAA | |
| | | CCTTTATGGCGCAGGAGCTATAGGAGGAAAACTGTTCGTT | |
| | | TTTTAATTTCTTACATGA | |
| NC_00 | Acanthocardia | TTGGCCTTTAATTGGGGTCCAGTATGAATGGGTTGACGAT | 175 |
| 8452 | tuberculata | GGAATAGCTGTCTTATAGGGGCTCATTAGAAATTTTCTTT | |
| | | TTGGTGAAAAGACCTAATTAAACTTAATAGACGAGAAGAC | |
| | | CCCGTCGAG | |
| LT630 | Tridacna | AGTAGCGTGATAAGTCGGCCTTTAATTGGGGTCTGGTATG | 176 |
| 276 | maxima | AAAGGGTTGACGTCGGAAAAGCTGTCTCAGAAAGATTAAA | |
| | | TGAAATTTACTTTTAAGTGAAAAGGCTTAGATGGGAGTAA | |
| | | AAGACGAGAAGGCCCTGTCGAGC | |
| NC_02 | Lutraria | TGTAGCGTAATAATTAGCCTTTTAAAAGGAGGGAGGAATG | 177 |
| 3384 | rhynchaena | AATGGTTTGACGAGAGACCTGCTGTCTCGGAAGTAAATTC | |
| | | TAAAGTTTTCTTTTAAGTGAAAAGACTTAGATTTTTAAAT | |
| | | AAGAAGAGAAGACCCCGGTGAGC | |
| NC_02 | Meretrix | TACCGCAGGGATAACAGCGTAATTTTTTTTTGTAAATTTT | 178 |
| 2924 | lyrata | ATTGGAAAAAAAGTTTACGACCTCGATGTTGAATTAAGAA | |
| | | AACTTTATGGCTTAGGCGCTATAATGAGTAAAACTGTTCG | |
| | | TTTTTTAATTTCTTACATGA | |
| NC_02 | Arctica | GGTAGCGTAATCAGTTGTCCTTTAATTGGGGAAAGGAATG | 179 |
| 2709 | islandica | AATGGTTTGACGAAAGGGTAGCTGTTTCAGAGGTAGATAA | |
| | | GGAAGTTTTCTTTCAGGTGAAAAGACCTGAGTTTTTGTAA | |
| | | AAGACGAGAAGACCCCGTCGAAC | |
| NC_01 | Solen | CTTAATTGGGGGCTAGAATGAAGGGTTTGACGTAAAAAAA | 180 |
| 7616 | strictus | TTTGTCTCCAAAATAGTAGTATGAAGTTTTCTTTTCAGTG | |
| | | AAAAGCCTGAAATTGTGATAAAAGACGAGAAGACCCTGTC | |
| | | GAGC | |
| NC_01 | Paratapes | GGTAGCGCGATAATTTGTCTCTTAATTGGAGAATGGTATG | 181 |
| 6891 | undulatus | AAAGGTTTGTCGCAGACTGGTTGTCTCTGAAGAAATAGAT | |
| | | GAAGTTTCCTTTTAAATGAAAAGGTTTAAATTTTTGTAAA | |
| | | AGACGAGAAGACCCTGCCGAG | |
| NC_01 | Paratapes | GGTAGCGCGATAATTTGTCTCTTAATTGGAGAATGGTATG | 182 |
| 6890 | textilis | AAAGGTTTGTCGCAGATTAATTGTCTCTAAAAAAAACAAT | |
| | | GAAGTTTCCTTTCAAATGAAAAGGTTTAAATTTTTGTAAA | |
| | | AGACGAGAAGACCCTGTCGAG | |
| NC_01 | Paphia | GGTAGCGCGATAATTTGTCTCTTAATTGGAGAATGGTATG | 183 |
| 6889 | amabilis | AAAGGTTTGACGTAGATCGATTGTCTCTATAAAAAAAAAA | |
| | | TGAAGTTTCCTTTTAAATGAAAAGGTTTAAATTTTTGTAA | |
| | | AAGACGAGAAGACCCTGCCGAGC | |
| NC_01 | Solen | CTTAATTGGGGGCTAGAATGAAGGGTTTGACGTAAAAAAG | 184 |
| 6665 | grandis | TTTGTCTCCAAAATAAAAGTATGAAGTTTTCTTTTCAGTG | |
| | | AAAAGTCTGATATAATGGTAAAAGACGAGAAGACCCTGTC | |
| | | GAGC | |
| NC_03 | Lutraria | TGTAGCGTAATCATTAGCCTTTTAAAAGGAGGGAGGAATG | 185 |
| 6766 | maxima | AATGGTTTGACGAGGGACCTGCTGTCTCAGGAGTAATTTC | |
| | | TAAAGTTTTCTTTTAAGTGAAAAGACTTAAATTTTTAAAT | |
| | | AAGAAGAGAAGACCCCGGTGAGC | |
| NC_03 | Donax | GTATTGAGGTAGCATAATAATTAGTCTTTTAATTGGGGGA | 186 |
| 5987 | vittatus | TGGAATGAATGGTTGAATGATGGGGTGTCTTTATTAGTAG | |
| | | TATAAATTCGAAGTTTTCTTTTAAGTGAAAAGACTTAAAT | |
| | | TTTTTTGTTAGACGATAAGACCCTATTGAGC | |
| NC_03 | Donax | GCGTTACGGTAGCATAATAAGTTGTCTTTTAATTGGGGGA | 187 |
| 5986 | variegatus | TAGTATGAATGGTTTGATGATGGGGTAGCTTTATTAATAA | |
| | | TATACATGTGAAGTTTACTTTTAAGTGAAAAGGCTTAAGT | |
| | | TGTTTTGTTAGACGATGAGACCCTATCGAGC | |
| NC_03 | Donax | GTGTTAAGGTAGCATAATAAATAGTCTCTTAATTGGGGGA | 188 |
| 5985 | trunculus | TAGAATGAATGGTTGAATGATGGGATAGCTTTATTAGTAA | |
| | | TATAAATATGAAGTTTACTTTTAAGTGAAAAGGCTTAAAT | |
| | | TTTATTGTTAGACGATAAGACCCTATTGAGC | |
| NC_03 | Donax | GTGTTAAGGTAGCATAATAATTAGTCTTTTAATTGGGGGA | 189 |
| 5984 | semistriatus | TGGAATGAATGGTTGAATGATGGGGTGTCTTTATTAGTAG | |
| | | TATAAATTTGAAGTTTTCTTTTAAGTGAAAAGGCTTAAAT | |
| | | TTTTTTGTTAGACGATAAGACCCTATTGAGC | |
| NC_03 | Ruditapes | GGTAGCGCGATAATTTGTCTTTTAATTGAAGAATGGTAGA | 190 |
| 5757 | decussatus | AAGGTTGTCGCAAGTTGCTTGTCTCTGAAAAAGAAAGCGA | |
| | | AGTTTCCTTTTAAATGAAAAGGTTTAAGTAAAGATAAAAG | |
| | | ACGAGAAGACCCTATTGAGC | |
| NC_03 | Cerastoderma | TTGGCCTTTAATTGGGGTCCGGTATGAATGGGTTGACGAT | 191 |
| 5728 | edule | AGAAAAGCTGTCTCATTGGAATATATTGAACTTTTCTTCT | |
| | | CGGTGAAAAGACCGCGCTTCTGTTAAAAGACGAGAAGACC | |
| | | CCGTCGAG | |
| NC_02 | Tridacna | AGTAGCGTGATAAGTCGGCCTTTAATTGGGGTCTGGTATG | 192 |
| 6558 | squamosa | AAAGGGTTGACGTCGGAAAAACTGTCTCGGAAGAACTAAG | |
| | | TGAAATTTACTTTTAAGTGAAAAGGCTTAAATTAAAGTAA | |
| | | AAGACGAGAAGACCCTGTCGAGC | |
| NC_03 | Ruditapes | GGTAGCGCGATAATTTGTCTCTTAATTAGAGAATGGTATG | 193 |
| 1332 | philippinarum | AAGGGTTAGACGTGGGATTATTGTCTCTAAAAAAKAKAGT | |
| | | TAAGTTTCCTTTTAAATGAAAAGGTTTAAATTTATTAAAA | |
| | | GACGAGAAGACCCTGTCGAGC | |
| NC_02 | Mactra | GGTAGCGCAATAAATTGTCCTTTAATTGGGGAAATGAATG | 194 |
| 5510 | chinensis | AAAGGGTTGACGAGGGGGAAGCTATCTTTTTGGTATAGTT | |
| | | TGAAGTTTTCTTTTATGTGAAAAGACTTAGATTTTATAAT | |
| | | TAGAAGAGAAGACCCCGGCGAGC | |
| NC_03 | Tridacna | AGTAGCGTGATAAGTCGGCCTTTAATTGGGGTCTGGTATG | 195 |
| 9945 | derasa | AAGGGGTTGACGTCGGAGAAACTGTCTCGGAAAAATATGG | |
| | | TGAAATTTACTTTTAAGTGAAAAGGCTTAAATAAAAGTAA | |
| | | AAGACGAGAAGGCCCTGTCGAGC | |
| NC_04 | Mercenaria | GGTAGCGTGATAAGTTGTCTTTTAATTGGAGAATGGAATG | 196 |
| 8487 | mercenaria | AAGGGTTTGACGTGAAATTTCTTTTTTTATAAAAGGTACT | |
| | | AAAGTTTCCTTCTAAGTGAAAAGGCTTAGGTTTTTGAAAA | |
| | | AGACGAGAAGACCCTGTCGAAC | |
| NC_05 | Tridacna | AGTAGCGTGATAAGTTGGCCTTTAATTGGGGTCTGGTATG | 197 |
| 0683 | gigas | AAGGGGTTGACGTCGGAGAAACTGTCTCGGAAAGATAAAA | |
| | | TGAAATTTACGTCCTAGTGAAAAGGCTGGGATGGCTGTAA | |
| | | AAGACGAGAAGACCCTGTCGAGC | |
| NC_02 | Mactra | GGTAGCGCAGTCAATTGTCCTTTAATTGGGGAAAGGAATG | 198 |
| 1375 | antiquata | AAAGGGTTGACGAGGGGGAGGCTGTCTTTGTAATATAATT | |
| | | TGAAATTTTCTTTTACGTGAAAAGACTTAAATTTTAAAAT | |
| | | AAGAAGAGAAGACCCCGGCGAGC | |
| NC_04 | Helix | TGACTGTGCAAAGGTAGCATAATCAGTTGGCTTTTAATTA | 199 |
| 1247 | pomatia | GAGTCTGGTATGAACGAAAATATGGGTAGTAACTGTCTTA | |
| | | GCACATTTTTTCGAACTTATTAACCAAGTGCAAATGCTTG | |
| | | TAATTTAACAATAGACGAGAAGACCCTAGAAACTT | |
| NC_02 | Achatina | TGACTGTGCAAAGGTAGCATAATAATTTGTCCTCTAATTA | 200 |
| 4601 | fulica | AGGTCTGGAATGAAGGGGGACACAGGGGAGAGCTGTCTCC | |
| | | AATAAGGTTAATTTAACTTTCTTATCAGGTGAAAATTCCT | |
| | | GAGCCTCCGATGAAAGACGAGAAGACCCTTAGAGTTT | |
| NC_02 | Helix | TGACTGTGCAAAGGTAGCATAATCAGTTGGCCTATAATTG | 201 |
| 1747 | aspersa | AGGTCTGGTATGAACGAATAATTGGGTAGTAACTGTCTTA | |
| | | ATGTATTCTTACAAATTTAGTAAGCAAGTGAAAATACTTG | |
| | | CTAAATTATAAAAGACGAGAAGACCCTAGAAACTT | |
| KT211 | Helix | TGACTGTGCAAAGGTAGCATAATCAGTTGGCTTTTAATTA | 202 |
| 748 | thessalica | GAGTCTGGTATGAACGAAAATATGGGTAGTAACTGTCTTA | |
| | | GCACATTTCTTTGAACTTGTTAAACAAGTGCAAATGCTTG | |
| | | TAATTAAAAAATAGACGAGAAGACCCTAGAAACTT | |
| KR705 | Helix | TGACTGTGCAAAGGTAGCATAATCAGTTGGCTTTTAATTA | 203 |
| 016 | pomatia | GAGTCTGGTATGAACGAAAATATGGGTAGTAACTGTCTTA | |
| | | GCATATTTTTTCGAACTTATTAAACAAGTGCAAATGCTTG | |
| | | TAATTTAACAATAGACGAGAAGACCCTAGAAACTT | |
| KR705 | Helix | TGACTGTGCAAAGGTAGCATAATCAGTTGGCTTTTAATTG | 204 |
| 014 | lucorum | GAGTCTGGTATGAACGAACACATGGGTGGCGACTGTCTAA | |
| | | ATATATTTTTCCTAACTTTTTAATCAAGTGAAAATACTTG | |
| | | TGCTAATAAAAAAGACGAGAAGACCCTAGAAACTT | |
| KR705 | Helix | TGACTGTGCAAAGGTAGCATAATCAGTTGGCTTTTAATTG | 205 |
| 012 | nicaeensis | GAGTCTGGCATGAACGAAAGCATGGGTGGCGACTGTCTTA | |
| | | ATATATTTTTTCTAATTTTTTAATCAAGTGAAAATACTTG | |
| | | TGTAAAATAAAAAAGACGAGAAGACCCTAGAAACTT | |
| JQ619 | Achatina | TGACTGTGCAAAGGTAGCATAATAATTTGTCCTTTAATTT | 206 |
| 236 | reticulata | AGGTCTGGAATGAATGAGAACACAGGGGAGAGCTGTCTCT | |
| | | AGWARGGTTAACTTAACTTCCTTATCAGGTGAAAATTCCT | |
| | | GAGCTTCTTTGGAAAGACGAGAAGACCCTTAGAGTTT | |
| EU912 | Helix | GACTGTGCAAAGGTAGCATAATCAGTTGGCCTATAATTGA | 207 |
| 833 | aspersa | GGTCTGGAATGAACGAAAAATAGGGTAGCAACTGTCTTAG | |
| | maxima | TATATTTTTTTAAACTTAGTTAGCAAGTGAAAATACTTGC | |
| | | AAATATACAAAAGACGAGAAGACCCTAGAAACTT | |
| GQ402 | Helix | GTCTGGTATGAACGAACATTTGGGTGACAACTGTCTAAAT | 208 |
| 391 | aperta | TAATTTTTCTAAACTTAGTTAGCAAGTGCAAATACTTGTA | |
| | | TGTATATAAAAGACGAGAAGACCCTAGAAACTT | |
| KR705 | Helix | TGACTGTGCAAAGGTAGCATAATCAGTTGACTCTTAATTA | 209 |
| 013 | albescens | GGGTCTGGTATGAAAGAAAATATGGGTAGTAACTGTCTTA | |
| | | ATATATTTATTTGAAATTAATAAGCAAGTGCAAATACTTG | |
| | | CATACATAAAAAAGACGAGAAGACCCTAGAAACTT | |
| MH130 | Tyrrhenaria | TGACTGTGCAAAGGTAGCATAATCAGTTGGCTTTTAATTA | 210 |
| 070 | ceratina | GAGTCTGGTATGAATGAAAATATGGGTGGTAACTGTCTCT | |
| | | GACATATTAATTTGAACTTTTTTTAGCAAGTGCAAATACT | |
| | | TGCGAGGGTAAAAAAGACGAGAAGACCCTAGAAACTT | |
| MF564 | Helix | TGACTGTGCAAAGGTAGCATAATCAGTTGGCTTTTAATTG | 211 |
| 117 | vladika | GAGTCTGGCATGAATGAAAATATGGGTAGTAACTGTCTTA | |
| | | GCATATTATTTGAACTTATTAAACAAGTGCAAATGCTTGT | |
| | | AATTACACAATAGACGAGAAGACCCTAGAAACTT | |
| KF246 | Pleurodonte | TGACTGTGCAAAGGTAGCATAATCAGTTGGCTTATAATTG | 212 |
| 957 | discolor | AAGTCTTGTATGAAGGAATAAATGGGTTACAACTGTCTCT | |
| | | ACAGATAAATAGCGAATTTACTTAATAAGTGAAAATACTT | |
| | | ATAGTTTTTTAAAAGACGAGAAGACCCTAGAAATTT | |
| KF246 | Pleurodonte | TGACTGTGCAAAGGTAGCATAATCAGTTGGCTTATAATTG | 213 |
| 963 | lychnuchus | AAGTCTAGTATGAATGAAGGAATGGGTGAGTCCTGTCTCC | |
| | | AGAAAAGAGGTGAACTTACCTAACAAGTGAAAATGCTTGT | |
| | | ACAAAGACAAAAGACGAGAAGACCCTAGAAATTT | |
| GQ402 | Erctella | CATATTGAGGTCTGGTATGATGAAATTTTGGGTAGAAACT | 214 |
| 402 | mazzullii | GTCTTAATATATTTTTTATAATTTAATATGCAAGTGAAAA | |
| | | TACTTGCTTTAATATAAAAGACGAGAAGACCCTAGAAACT | |
| | | T | |
| GQ402 | Erctella | GAAGTCTGGTATGAATGAACTTTGGGTAGTAACTGTCTTA | 215 |
| 397 | cephalae | ATATATTTTAAAAAATTTAATATGTAAGTGAAAATACTTA | |
| | ditana | CTTTAATATAAAAGACGAGAAGACCCTAGAAACTT | |
| KF246 | Pleurodonte | TGACTGTGCAAAGGTAGCATAATCAGTTGGCTTATAATTG | 216 |
| 958 | formosa | AAGTCTGGTATGAAGGAATAAATGGGTAGTTTCTGTCTTT | |
| | | AGGGGAACGACGAACTTACTCAGCAAGTGAAAATACTTGT | |
| | | ATATTAACAAAAGACGAGAAGACCCTAGAAATTT | |
| KR705 | Helix | TGACTGTGCAAAGGTAGCATAATCAGTTGACTCTTAATTG | 217 |
| 018 | christophi | GGGCCTGGGATGAATGAAATAATGGGGGGCAGCTTTCTCA | |
| | | GATATATTTCTATAAACTTGGTTAGTAGGTGCAAATACCT | |
| | | CTATGTAGATAAAAGACGAGAAGACCCTAGAAGCTT | |
| KR705 | Helix | TGACTGTGCAAAGGTAGCATAATCAGTTGGCTTTTAATTG | 218 |
| 019 | nordmanni | GAGTCTGGGATGAATGAAATAATGGGGGGCAACTTTCTCA | |
| | | AATATATTATTATGAATTTGGTTAGTAGGTGCAAATACCT | |
| | | ACATAAAAACAAAAGACGAGAAGACCCTAGAAGCTT | |
| KF246 | Pleurodonte | TGACTGTGCAAAGGTAGCATAATCAGTTGTCTTTTAATTG | 219 |
| 965 | nucleola | AAGTCTGGCATGAATGAATAAATGGGTAGGAACTGTCTCA | |
| | | AGAAAGAAACAAAAATTTACTTAGTAAGTGAAAATACTTA | |
| | | TGTTTTGACAAAAGACGAGAAGACCCTAGAAATTT | |
| KF246 | Pleurodonte | TGACTGTGCAAAGGTAGCATAATCAGTTGGCTTTTAATTG | 220 |
| 967 | parilis | AAGTCTAGTATGAAGGAACAAATGGGTGAAAGCTGTCTTT | |
| | | AGTAGAAAACAAGGAACTTACTTAGTAAGTGAAAATACTT | |
| | | ACAGTTAGATAATAGACGAGAAGACCCTAGAAATTT | |
| KF246 | Gonostomopsis | TGACTGTGCAAAGGTAGCATAATCAGTTGGCTTATAATTG | 221 |
| 969 | auridens | AAGTCTAGTATGAAAGAATAAAATGGGCGGGCGCTGTCTT | |
| | | CTTAAGGTATCTAGAACTTACTTAGTAAGTGAAAATACTT | |
| | | ACATATAAATAAAAGACGAGAAGACCCTTGAAGTTT | |
| KF246 | Caracolus | TGACTGTGCAAAGGTAGCATAATAATTTGGCTTTTAATTG | 222 |
| 970 | caracollus | GAGTCTGGAATGAATGGGTACATGGATATAGGCTGTCTTA | |
| | | TACTAATTACTTATAACTTTTTTAATTAAGTGAAAATACT | |
| | | TAAAGAAATATAATAGACGAGAAGACCCTAGAAATTT | |
| KF246 | Lacteolun | TGACTGTGCAAAGGTAGCATAATAAATTGGCTTATAATTG | 223 |
| 999 | aselenina | AAGTCGGGAATGAATGGATTAATGAATATAAACTGTCTTA | |
| | | TCTAAATTTTAATTAATTTTCTTAGTAAGTGAAAATTCTT | |
| | | ACAATGTGTTTTATTAGACGAGAAGACCCTTGAAATTT | |
| KF247 | Cernuella | TGACTGTGCTAAGGTAGCATAATCATTTGGCTTATAATTG | 224 |
| 012 | cisalpine | AAGTCTGGCATGAAAGAATTTATGGGTAACAACTGTCTCC | |
| | | TGTAATTATAGATGAACTTACTCAATAAGTGAAAATGCTT | |
| | | ATATAATCACAATAGACGAGAAGACCCTAGAAATTT | |
| KF247 | Cochlicella | TGACTGTGCAAAGGTAGCATAATCAATTGGCTTGTAATTT | 225 |
| 015 | acuta | AAGTCTGGTATGAATGTAGTTTTAGGCTGAAACTGTCTCT | |
| | | TTATATACATTGAAATTTACTTAGCCGGTGAAAATGCCGG | |
| | | CAGTTAAATAATAGACGAGAAGACCCTAGAAATTT | |
| KF247 | Disculella | TGACTGTGCAAAGGTAGCATAATCATTTGGCTTATAATTG | 226 |
| 017 | maderensis | AAGTCTAGTATGAATGAATTAATGGGTAAAAGCTGTCTCT | |
| | | TTATAATTAAGTAGAAATTACTTAGAGGGTGAAAATTCTC | |
| | | TCAATATCTTAATAGACGAGAAGACCCTAGAAATTT | |
| KF247 | Dialeuca | TGACTGTGCAAAGGTAGCATAATAAATTGGCTTATAATTG | 227 |
| 020 | nemoraloides | AAGTCTTGAATGAAAGGTAACACGAGTAAAATCTGTCTTT | |
| | | TAGAAATATTTTTAATTTACTTATTAAGTGAAAATTCTTA | |
| | | ATTTAAAAAATTAGACGAGAAGACCCTAGAAATTT | |
| KF247 | Monadenia | TGACTGTGCAAAGGTAGCATAATCAGTTGGCCTTTAATTG | 228 |
| 021 | fidelis | GGGTCTGGAATGAACGAAGATTTGGGTAATAGCTGTCTTT | |
| | | ATATGGTAAGCAAAACTTGCTTAGTAAGTGAAAATTCTTA | |
| | | CATGATAAGAAAGACGAGAAGACCCTAGAAATTT | |
| NC_00 | Cepaea | TGACTGTGCAAAGGTAGCATAATAATTTGGCTTATAATTG | 229 |
| 1816 | nemoralis | AAGTCTGGCTCGAAAGAATTAATGGGAAGCAGCTGTCTCC | |
| | | AGGGGTACTAGTACAAAATTAGTAAGTAAGTGAAAATACT | |
| | | TGCGGGAAGAAAATAGACGAGAAGACCCTAGAAGCTT | |
| KF247 | Sphincterochila | TGACTGTGCTAAGGTAGCATAATCAATTGACTCATAATTG | 230 |
| 040 | candidissima | GAGTCTAGCATGAATGAAAGAATGGGTAAACACTGTCTCT | |
| | | GTCTTACCGATTGAAATTACTAAGCAGGTGAAAATACCTG | |
| | | TATTCAAATAATAGACGAGAAGACCCTAGAAGACC | |
| KF247 | Microphysula | TAACTGTGCAAAGGTAGCATAATAATTTGGCTTATAATTG | 231 |
| 041 | ingersolli | AAGTCTAGAATGAAAGGAAGCATGGGCGAATTCTGTTTCT | |
| | | TAATTTTTTTTTTGAAATTAATAATAAGGTGAAAATTCCT | |
| | | TTTAAATTCTATTAGACGAGAAGACCCTACAGAGCT | |
| KF247 | Helicodonta | TGACTGTGCAAAGGTAGCATAATAATTTGGCTTATAATTG | 232 |
| 043 | obvoluta | AAGTCTGGTATGAATGAATATATGGGTACCAACTGTCTCC | |
| | | ACTATATAGCATGAATTTACTGATTGGGTGCAAATTCCCA | |
| | | TCGCAAGATAATAGACGAGAAGACCCTAGAAATTT | |
| NC_03 | Cernuella | TGACTGTGCTAAGGTAGCATAATCATTTGGCTTATAATTG | 233 |
| 0723 | virgata | AAGTCTGGCATGAAGGAATTTATGGGTAACAACTGTCTCC | |
| | | TATAACTGTAATGAACTTGCTAAATAAGTGAAAATACTTA | |
| | | TGTAACCATAATAGACGAGAAGACCCTAGAAATTT | |
| AF110 | Loligo | GGGACGAGAAGACCCTATTGAGCTTATAATTTTATTATTA | 234 |
| 075 | forbesii | TTATTATTTGTAATTGGTGTAATAATATATAAATTTTAAT | |
| | | TGGGGTGATTAAGGAATAAAAATTAATTAATATGATTAAA | |
| | | TAACTTCCTTAGAAATTATATTGTTAAATAAAGTAACCTA | |
| | | TARTTATTATAAAAATTTATAATCAGTATTGCTTATAATA | |
| | | TAGTTACCATAGGGATAACAGCGTAAT | |
| AB270 | Nototodarus | GGGACGAGAAGACCCTAATGAGCTTATAATTTTATTGTTA | 235 |
| 953 | sloanii | TGGTTTAATATATTTAAATGATAATATATAAATTTTAATT | |
| | | GGGGTGATTAAGGAATAAATTATGATTAATAACTTCCTTA | |
| | | GGTTAAATATATATTGATTAAGTAACCAATAAATATATTG | |
| | | CTTATAATATAGTTACCATAGGGATAACAGCGTAAT | |
| AB193 | Sepia | GGGACGAGAAGACCCTATTGAGCTTTAAATTTGAAGTATT | 236 |
| 802 | lorigera | GTATTTATTAATATAATATTTAAAAATTTTAATTGGGGTG | |
| | | ATTAAGGAATAAAAATTATAATATATTAAAAATTTAACTT | |
| | | CCTTAAATAATATATTGTTAAATAAAGTAACCAATAATAT | |
| | | TGCTTATATAAAGTTACCATAGGGATAACAGCGTAAT | |
| AB193 | Sepia | GGGACGAGAAGACCCTATTGAGCTTTAAATTTGGAATATT | 237 |
| 801 | pardex | ATATTTGTTAGTATAGTATTTAAAAAATTTTAATTGGGGT | |
| | | GATTAAGGAATAAAATTATAAGTATATTAAAATTTAACTT | |
| | | CCTTAAATAATATATTATTAAATAAAGTAACCAATAATAT | |
| | | TGCTTATATAAAGTTACCATAGGGATAACAGCGTAAT | |
| AB192 | Sepia | GGGACGAGAAGACCCTATTGAGCTTTAAATTTGGGAATAT | 238 |
| 323 | kobiensis | TATATTTATTAAGTATAATGTTTGAAAAATTTTAATTGGG | |
| | | GTGATTAAGGAATAAAATAAAATATATTAAAAATTTAACT | |
| | | TCCTTAGATAATATATTGTTAAATAAAGTAACCAATAATA | |
| | | TTGCTTATATAAAGTTACCATAGGGATAACAGCGTAAT | |
| AB191 | Rossia | GGACGAGAAAGACCCTATTGAGCTTGTAATTTTATTTTAT | 239 |
| 138 | pacifica | TGTTTATTAAGGATAGTAAAGTATAAATTTTAATTGGGGT | |
| | | GATTAAGGAATAAGATAATATAAGATAACTTCCTTAAAGG | |
| | | TTAGTATAGGTGTATGAAGTAACCAATAAATTATATTGCT | |
| | | TATATTATAGTTACCATAGGGATAACAGCGTAAT | |
| AB191 | Berryteuthis | GGGACGAGAAGACCCTATTGAGCTTATAATTTTATTGCTA | 240 |
| 136 | magister | CTAAATTATTATTAGTGTAGTAGTATATAAATTTTAATTG | |
| | | GGGTGATTAAGGAATAATTTAGTTTAGTAACTTCCTTAAA | |
| | | TTTACATATTGGTGGATAAAAGTAACCCATAAAGTATTGC | |
| | | TTATAATATAGTACCATAGGGATAACAGCCGAAT | |
| AJ252 | Eledone | GGAACGAGAAGACCCTATTGAGCTTAATATTAAAATATAA | 241 |
| 763 | massyae | ATTTTGTTAGTAATAAATTAATAAAGTTTATTAATTTTAA | |
| | | ATTTTGATTGGGGTGATCGAGGAATATAAATAATTAAATT | |
| | | TAACTTCCTAATATTATATATTTATAAATAAAGAAACCAA | |
| | | ATATTTTGCTTAGAAGGAAGTTACCATAGGGATTACAGCG | |
| | | TGAT | |
| AF369 | Sepia | GGGACGAGAAGACCCTATTGAGCTTAAATTTAATTACATA | 242 |
| 957 | robsoni | ATTAAATTATGTTTTGATAAATTTTAATTGGGGTGATTAA | |
| | | GGAATAAAAATATTATATAGTAACTTCCTTAATAGTTAAT | |
| | | ACTGTTGGGTTAATTAACCAATAATATTGCTTATAATAAA | |
| | | GTTACCATAGGGATAACAGCGTAAT | |
| AF110 | Loligo | GGGACGAGAAGACCCTACTGAGCTTGTAATTTTATTATTA | 243 |
| 081 | reynaudii | CTGCTATTTGTAATTGGGGCAGTAATATATAAATTTTAAT | |
| | | TGGGGTGATTAAGGAATAAAATAAATATATCTTAGTAACT | |
| | | TCCTTATAAAGTATATTGTTAGATGAAGTAACCAATTAAA | |
| | | GTTACATGATATTGTAATAAATATTGCTTATAATATAGTT | |
| | | ACCATAGGGATCACAGCGTAAT | |
| AF110 | Doryteuthis | GGGACGAGAAGACCCTATTGAGCTTATAATTTTATTATTG | 244 |
| 079 | (Amerigo) | TCAGTATTGTTAATTTTTATTGGCACTATATAAATTTTAA | |
| | pealeii | TTGGGGTGATTAAGGAATAAGGTTTATATATTTCAGTAAC | |
| | | TTCCTTAGTAGTAGATAAATTGTTAGATAAAGTAACCAAT | |
| | | ATTATTAGATTTATTGCTTATACTATAGTTACCATAGGGA | |
| | | TAACAGCGTAAT | |
| AF110 | Doryteuthis | GGGACGAGAAGACCCTATTGAGCTTATAATTTTATTATTA | 245 |
| 076 | (Amerigo) | CCAATATTATTAATTGTTAGTGGTAATATATAAATTTTAA | |
| | gahi | TTGGGGTGATTAAGGACATAAAAAGTTTATTATTTAGTAA | |
| | | CTTCCTTAGTAAATATAAATTGTTAAATAAAGTAACCAAT | |
| | | ATAAATAAATATTATTGCTTATATTATAGTTACCATAGGG | |
| | | ATAACAGCGTAAT | |
| AY293 | Sepiola | GGGACGAGAAGACCCTATTGAGCTTATAATTTTATTATTA | 246 |
| 671 | rondeletii | TTATTATATGTAATAATAAATGAATTTTAATTGGGGTGAT | |
| | | TAAGGAATAAATTAGTATTAGTTAGTAACTTCCTTAAATA | |
| | | GATAAATAGATGTATAAATTAACCAATAATTATATTGCTT | |
| | | TTATTAAAGTTACCATAGGGATAACAGCGTAAT | |
| AY293 | Sepiola | GGGACGAGAAGACCCTATTGAGCTTGTAATTTTATTAGTA | 247 |
| 670 | robusta | TTATTAATATAATATTTAATAAATTTTAATTGGGGTGATT | |
| | | AAGGAATAAATTAATATTATTAGTAACTTCCTTAAGTAAT | |
| | | AGGATAGGTGTATAAATTAACCAATAATTGTATTGCTTTT | |
| | | ATCAAAGTTACCATAGGGATAACAGCGTAAT | |
| AY293 | Sepiola | GGGACGAGAAGACCCTATTGAGCTTATAATTTTATTAGTA | 248 |
| 669 | intermedia | TTATTAATATAATATTTAATAAATTTTAATTGGGGTGATT | |
| | | AAGGAATAAATTAATATTATTAGTAACTTCCTTAAGTAAT | |
| | | AGGATAGGTGTATAAATTAACCAATAATTGTATTGCTTTT | |
| | | ATCAAAGTTACCATAGGGATAACAGCGTAAT | |
| AY293 | Adinaefiola | GGGACGAGAAGACCCTATTGAGCTTTTAATTTTGATATAT | 249 |
| 668 | ligulata | TATATTTATATAATGTTGAATAAATTTTAATTGGGGTGAT | |
| | | TAAGGAATAAATATAATTAATTAGTATCTTCCTTAAAATA | |
| | | ATAGTATAGATATATAAATTAACCAATAATTATATTGCTT | |
| | | ATATTAGAGTTACCATAGGGATAACAGCGTAAT | |
| AM088 | Sepia | GGGACGAGAAGACCCTATTGAGCTTAAATTTAATTTTATT | 250 |
| 007 | smithi | AATTATGATATTAGTAAATAATAAATTTTAATTGGGGTGA | |
| | | TTAAGGAATAAATATAATAATAAGTAATAACTTCCTTAAA | |
| | | TTATTAAACTGTTAGGTGAAGTAACCAATAATATTGCTTA | |
| | | TATTAAAGTTACCATAGGGATAACAGCGTAAT | |
| AM088 | Sepia | GGGNCGAGAAGACCCTATTGAGCTTAAATTAATNGTATAT | 251 |
| 005 | elliptica | TAAATTAAGATTAATATATTAATAATTTTAATTGGGGTGA | |
| | | TTAAGGAATAAAAAATATTGTAAAATGATAACTTCCTTAA | |
| | | ATTTAATACTGTTAGGTAGAGTAACCAATATTTATTGCTT | |
| | | ATATTAAAGTTACCATAGGGATAACAGCGTAAT | |
| AJ252 | Eledone | AGGGACAAAAGACCCTATTGAGCTTTTATTATTTTTTAAA | 252 |
| 765 | palari | TTATTATTAATAAGAAATTTATGCAAATAATTTGATTGGG | |
| | | GTGATCAAGGAATAAAGAAATAACTTCCTTAAATATTAAG | |
| | | GTTTAATAGGAAAATAAACCAAATTTTTGCTTTTATGATG | |
| | | AGTTACCATAGGGATAACAGGGTAAT | |
| AJ252 | Eledone | GGGACGAGAAGACCCTATTGAGCTTAATGTTAAATTATAA | 253 |
| 764 | moschata | ATAATTTAATGATCTTATTATGTTTTACATTATTTATAAA | |
| | | ATTTAAATTTTGATTGGGGTGATCAAGGAATAAAAGTAGT | |
| | | GTAGTGTTTATAACTTCCTTATAGGATTTATGAGTTATTA | |
| | | ATGGTATAATAAACCAAATATTTTGCTTATAAGTAAGTTA | |
| | | CCATAGGGATAACAGCGTAAT | |
| EU735 | Sepiola | GGGACGAGAAGACCCTATTGAGCTTATAATTTTATTAGTA | 254 |
| 193 | affinis | TTATTAATATAATATTTAATAAATTTTAATTGGGGTGATT | |
| | | AAGGAATAAATTAATATTATTAGTAACTTCCTTAAGTAAT | |
| | | AGGATAGGTGTATAAATTAACCAATAATTGTATTGCTTTT | |
| | | ATCAAAGTTACCATAGGGATAACAGCGTAAT | |
| EU735 | Rossia | GGGACGAGAAGACCCTACTGAGCTTGTAATTTTATTTTAT | 255 |
| 192 | palpebrosa | TATTTTTAGGTAGTAGGGTATAAATTTTAATTGGGGTGAT | |
| | | TAAGGAATAAAGTGTATTAGGGTAACTTCCTTAAGTGATA | |
| | | GTATATGTGTATTAAGTAACCAATAAATTATATTGCTTAT | |
| | | ATTATAGTTACCATAGGGATAACAGCGTAAT | |
| AY681 | Gonatus | GGGACGAGAAGACCCTATTGAGCTTATAATTTTATCATTA | 256 |
| 045 | madokai | TTAGGTTTATTATTAATATAGTGATGTATAAATTTTAATT | |
| | | GGGGTGATTAAGGAATAAATTTAATTTAGTAACTTCCTTA | |
| | | AGTTTTATATTGTTGAATAAAGTAACCAATAAATTATTGC | |
| | | TTATAATATAGTTACCATAGGGATAACAGCGTAAT | |
| AY681 | Gonatus | GGGACGAGAAGACCCTATTGAGCTTATAATTTTATCATTA | 257 |
| 038 | kamtschaticus | TTAGATTTATTATTAATATAGTGATGTATAAATTTTAATT | |
| | | GGGGTGATTAAGGAATAAATTTAATTTAGTAACTTCCTTA | |
| | | AGTTCTATATTGTTGAATAAAGTAACCAATAAATTATTGC | |
| | | TTATAATATAGTTACCATAGGGATAACAGCGTAAT | |
| AY616 | Eledone | GGGACGAGAAGACCCTATTGAGCTTGATATTAAAATATAA | 258 |
| 973 | cirrhosa | ATTTTTTTTAAAGTTAATATAATTATGTTATGGGTTTAAA | |
| | | TTTTGATTGGGGTGATCAAGGAATAAAAAAGATTAATGTA | |
| | | TATATATATTAACTTCCTTATATATAAATATTTATAGATA | |
| | | AATAAACCGAGAGTTTTGCTTAAAAGTAAGTTACCATAGG | |
| | | GATAACAGCGTAAT | |
| AY377 | Sepia | GGGAAGAGAAGACCCTATTGAGCTTTAATTTTAATAGGTT | 259 |
| 630S | elegans | ATAATAAATATTATAGTAAAATAGAAATTTTAATTGGGGT | |
| | | GATTAAGGAATAAAAAAGTAACTTCCTTAAAAATAAAATT | |
| | | GTTAGGTTAAGTAACCAATTGTATTGCTTATATAAAGTTA | |
| | | CCATAGGGATAACAGCGTAAT | |
| AY293 | Rossia | GGGACGAGAAGACCCTAGTGAGCTTATAATTTTATTTTGT | 260 |
| 676 | bipillata | TGTTAGTAAATAGTAGAGATGAAATTTTAATTGGGGTGAT | |
| | | TAAGGAATAAAATATGTTTATATAACTTCCTTAGATTTGG | |
| | | TATAGGTATATAAAGTAACCAATAAATATATTGCTTATAT | |
| | | TATAGTTACCATAGGGATAACAGCGTAAT | |
| AY293 | Sepiola | GGGACGAGAAGACCCTATTGAGCTTATAATTTTATTATTA | 261 |
| 672 | atlantica | TTATATGTAATAATAAATGAATTTTAATTGGGGTGATTAA | |
| | | GGAATAATTAGTATTAGTTAGTAACTTCCTTAAATAGATA | |
| | | AATAGATGTATAAATTAACCAATAATTATATTGCTTTTAT | |
| | | TAAAGTTACCATAGGGATAACAGCGTAAT | |
| KF266 | Lolliguncula | GGGACGAGAAGACCCTATTGAGCTTGTAATTTTATTGTTA | 262 |
| 732 | (Lolliguncula) | TTATTATATTAATTGTTAATGGTAATATATAAATTTTAAT | |
| | panamensis | TGGGGTGATTAAGGAATAAAATTTTATATTAGTAACTTCC | |
| | | TTAATAAGATGATTGTTAGATAAAGTAACCAATATTATTA | |
| | | AATATTATTGCTTATATTGTAGTTACCATAGGGATAACAG | |
| | | CGTAAT | |
| KC792 | Octopus | GGGACGAAAAGACCCTATTGAGCTTATATTATTTTGTAAA | 263 |
| 312 | maya | TTATATGATTGTTGTTTATTGGAATTAATTTTGATTGGGG | |
| | | TGATCAAGGAATAAATTATTATTAAAATTAACTTCCTTAA | |
| | | TTAATTATTTTATGGAGAAATAAACCAAATTTTTTGCTTA | |
| | | GATGATAAGTTACCATAGGGATAACAGCGTAAT | |
| HQ733 | Illex | GGGACGAGAAGACCCTACTGAGCTTATAATTTTATTATCA | 264 |
| 953 | illecebrosus | CATATATTTATGTATAATGTGCTATATAAATTTTAATTGG | |
| | | GGTGATTAAGGAATAATATAGCTATTTTATAACTTCCTTA | |
| | | GATATTATATTGTTGAATTAAGTAACCGATTAATTAATAG | |
| | | AATTGTATTATGTATTGCTTATAATATAGTTACCATAGGG | |
| | | ATAACAGCGTAAT | |
| GQ412 | Nototodarus | GGGACGAGAAGACCCTAATGAGCTTATAATTTTATTGTTG | 265 |
| 305 | gouldi | TGGTTTAATATATTTAAATGATAATATATAAATTTTAATT | |
| | | GGGGTGATTAAGGAATAAATTATGATTAATAACTTCCTTA | |
| | | GGCTGAATATATATTGATTAAGTAACCAATAAATATATTG | |
| | | CTTATAATATAGTTACCATAGGGATAACAGCGTAAT | |
| EU735 | Gonatopsis | GGGACGAGAAGACCCTATTGAGCTTATAATTTTATCATTA | 266 |
| 264 | octopedatus | TTAGATTTATTATTAGTGTAGTGTGTATAAATTTTAATTG | |
| | | GGGTGATTAAGGAATAATTTAATTTATTAACTTCCTTAAG | |
| | | TTACATATTGTTGAATAAAGTAACCAATAAATTATTGCTT | |
| | | ATAATATAGTTACCATAGGGATAACAGCGTAAT | |
| EU735 | Illex | GGGACGAGAAGACCCTAATGAGCTTATAATTTTATTATTA | 267 |
| 218 | coindetii | TTATTTAATATATTTAAATAATAATATATAAATTTTAATT | |
| | | GGGGTGATTAAGGAATAAAGAATTATATAATAACTTCCTT | |
| | | AGATTAAATATATATAGATAAAGTAACCAATAATATATTG | |
| | | CTTATAATATAGTTACCATAGGGATAACAGCGTAA | |
| EU735 | Berryteuthis | GGGACGAGAAGACCCTAATGAGCTTATAATTTTATTGTTG | 268 |
| 238 | anonychus | TTAAATTTATTATTAGTAAAGCAGCATATAAATTTTAATT | |
| | | GGGGTGATTAAGGAATAAATTAATTTAGTAACTTCCTTAT | |
| | | ATTTAAATATTGTTAAATAAAGTAACCAATAAATTATTGA | |
| | | TTATAATATAGTTACCATAGGGATAACAGCGTAAT | |
| EU735 | Gonatus | GGGACGAGAAGACCCTATTGAGCTTATAATTTTATCATTA | 269 |
| 210 | fabricii | TTAGATTTATTATTAGTGTAATGTTGTATAAATTTTAATT | |
| | | GGGGTGATTAAGGAATAATATTAATTTAGTAACTTCCTTA | |
| | | AGTTATATATTGTTGAATAAAGTAACCAATTAAATTATTG | |
| | | CTTATAATATAGTTACCATAGGGATAACAGCGTAAT | |
| KR259 | Lusepiola | GGGACGAGAAGACCCTATTGAGCTTGTAATTTTATTTTAT | 270 |
| 947 | birostrata | TGTTTATTAGGATAGTAAAGTATAAATTTTAATTGGGGTG | |
| | | ATTAAGGAATAAGATAATATAAGATAACTTCCTTAAAGGT | |
| | | TAGTATAGGTGTATGAAGTAACCAATAAATTATATTGCTT | |
| | | ATATTATAGTTACCATAGGGATAACAGCGTAAT | |
| KJ605 | Octopus | GGACGAAAANGACCCTATTGAGCTTTTGTTATTATATAAA | 271 |
| 235 | tetricus | TATACATATAAATGTATAATTTATTTATAAATAACTTTGG | |
| | | TTGGGGTGATCAAGGAATAAAAATAATTTAAAATGTTGTG | |
| | | TTAACTTCCTTAATTTTGAGTTTAATTGAAGAATAAACCA | |
| | | AGTTTTTTGCTTAGAAGATAAGTTACCATAGGGATAACAG | |
| | | CGTAAT | |
| KF854 | Uroteuthis | GGGACGAGAAGACCCTATTGAGCTTGTAATTCTGTTATTA | 272 |
| 042 | (Photololigo) | TTATGTTTATTAAATATCATTAATAGTATATGAATTTTAA | |
| | sibogae | TTGGGGTGATTAAGGAATAAATAATTTATAATATATAACT | |
| | | TCCTTAGAGTATTATATTGTTGAATGAATTAACCGATATA | |
| | | ATTAACACAGGTTGTGTTGTGTATTGCTTATAATATAGTT | |
| | | ACCATAGGGATAACAGCGTAAT | |
| KF854 | Doryteuthis | GGGACGAGAAGACCCTATTGAGCTTATAATTTTATTGTTG | 273 |
| 023 | (Doryteuthis) | TCAGTGAGTATTAATTTCACTGTTGGTATATAAATTTTAA | |
| | pleii | TTGGGGTGATTAAGGAATAAAGTTTATATATTAATAACTT | |
| | | CCTTAGAGTTAATATTATTGTTAGATAAAGTAACCAATAA | |
| | | ATTGTTAAGATTATTGCTTATACTATAGTTACCATAGGGA | |
| | | TAACAGCGTAAT | |
| KF854 | Doryteuthis | GGGACGAGAAGACCCTATTGAGCTTATAATTTTATTGTCA | 274 |
| 021 | sanpaulensis | TCAAGATTGTTATGTAACTGGTGACATATAAATTTTAATT | |
| | | GGGGTGATTGAGGAATAAAGTTTTATTGTGGAAGTAACTT | |
| | | CCTTAGTAATGCATATTGTTAAATGAAGTAACCAATAATT | |
| | | AATTACTATTGCTTATACTATAGTTACCATAGGGATAACA | |
| | | GCGTAAT | |
| KF854 | Doryteuthis | GGGACGAGAAGACCCTATTGAGCTTATAATTTTATTACCG | 275 |
| 020 | (Amerigo) | TTAGTAGTGATTAAAATTTTACTAAGGGTATATAAATTTT | |
| | surinamensis | AATTGGGGTGATTAAGGAATAAAAGCTTATGTATTAATAA | |
| | | CTTCCTTAGAAATAAATAAAAAATTGTTAGATAAAGTAAC | |
| | | CAATAAATTGTTAAAATTATTGCTTATACTATAGTTACCA | |
| | | TAGGGATAACAGCGTAAT | |
| KF373 | Octopus | GGGACGAAAAGACCCTATTGAGCTTGTATTATTTAACAAA | 276 |
| 761 | hubbsorum | TTATATGATTGTTATTTGTTTGAATTAATTTTGATTGGGG | |
| | | TGATCAAGGAATAAATTTTATTTAGTTTAACTTCCTTAAT | |
| | | TAATTGTTTTATAGAAGAATAAACCAAAGTTTTTGCTTAG | |
| | | AAGATAAGTTACCATAGGGATAACAGCGTAAT | |
| MG01 | Macrotritopus | GGGACGAAAAGACCCTATTGAGCTTGTGTTAATAAATAAA | 277 |
| 0490 | defilippi | TACTANNNTATATATATATANNNNNNTATATATATATATA | |
| | | TTANNTTTGTGAGTTTTAAATTTGGTTGGGGTGATCAAGG | |
| | | AATAAAATTAAAATAAAANNNNNNNNNNNNGTAACTTCCT | |
| | | TAAAAATNNNAATATTAATGGGTTAATAAACCAAGTTTTT | |
| | | TGCTTATAAGGTAAGTTACCATAGGGATAACAGCGTAAT | |
| MF040 | Octopus | GGGACGAAAAGACCCTATTGAGCTTATATTATTTTATAAA | 278 |
| 832 | insularis | TTATATGATTGTTGTTTATTTAGAATAATTTTGATTGGGG | |
| | | TGATCAAGGAATAAGATTGTTTAAATTAACTTCCTTAATT | |
| | | AATTTGTTTTGTGGAAAAATAAACCAAATTTTTTGCTTAG | |
| | | AAGATAAGTTACCATAGGGATAACAGCGTAAT | |
| LC121 | Loliolus | GGGACGAGAAGACCCTATTGAGCTTATAATTTTATTACTA | 279 |
| 083 | (Nipponol | TTATGTTTGTTAAATTCATCATTGTAGTATATAAATTTTA | |
| | oligo) | ATTGGGGTGATTAAGGAATAAGTTTTTTATAATGTAACTT | |
| | sumatrensis | CCTTAGATTAATATATTGTTAGATTAAGTAACCGATGTGA | |
| | | TTAACATAATATATGTTATGTATTGCTTATAATATAGTTA | |
| | | CCATAGGGATAACAGCGTAAT | |
| LC121 | Sepia | GGGACGAGAAGACCCTATTGAGCTTAAATTTAAAAATATT | 280 |
| 068 | stellifera | AAATAATTTAATATTTTGATAAATTTTAATTGGGGTGATT | |
| | | AAGGAATAAAATAATAGGTATTATTAAATATAGTAACTTC | |
| | | CTTAAATTTAATACTGTTAGGTTAAGTAACCAATGTAATA | |
| | | TTGCTTATAATAAAGTTACCATAGGGATAACAGCGTAAT | |
| LC121 | Sepia | GGGACGAGAAGACCCTATTGAGCTTGAATTTAATTATATT | 281 |
| 066 | recurvirostra | ATTTTAATAATATTTTGATAAATTTTAATTGGGGTGATTA | |
| | | AGGAATAAAAATTTATATATATTATGTAGTAACTTCCTTA | |
| | | ATTATTATACTGTTAGGTTAAGTAACCAATAATATTGCTT | |
| | | ATAATAAAGTTACCATAGGGATAACAGCGTAAT | |
| LC121 | Sepia | GGGACGAGAAGACCCTATTGAGCTTTAATTTAATTATATA | 282 |
| 063 | madokai | ATATAGTTTGTATTTTGTTAAATTTTAATTGGGGTGATTA | |
| | | AGGAATAAGATTAGTATTAATTAGTAACTTCCTTAAATTT | |
| | | TATTGTTACTGTTGGGTGAAGTAACCAATATTATTGCTTA | |
| | | TAATAAAGTTACCATAGGGATAACAGCGTAAT | |
| LC121 | Sepia | GGGACGAGAAGACCCTATTGAGCTTAAATTTAAAAATATT | 283 |
| 061 | kobiensis | AAATAATTTAATATTTTGATAAATTTTAATTGGGGTGATT | |
| | | AAGGAATAAAATAATAGGTATTATTAAATATAGTAACTTC | |
| | | CTTAAATTTAATACTGTTAGGTTAAGTAACCAATGTAATA | |
| | | TTGCTTATAATAAAGTTACCATAGGGATAACAGCGTAAT | |
| NC_02 | Amphioctopus | GGGACGAAAAGACCCTATTGAGCTTTATATTATACTTTAA | 284 |
| 9702 | aegina | AATTATTTATTGTTTTTATTTAAATATAATTTTGGTTGGG | |
| | | GTGATCAAGGAATAAATAATTAATTTTAACTTCCTTAATT | |
| | | TGTTTATAGTTATATGAATAAACCAAATTTTTTGCTTAGA | |
| | | AGATAAGTTACCATAGGGATAACAGCGTAAT | |
| NC_00 | Sepia | GGGACGAGAAGACCCTAATGAGCTTAAATTTTATTTTATT | 285 |
| 7895 | officinalis | ATATATATTTTATAGTAAAAAAGAAGTTTTAGTTGGGGTG | |
| | | ATTAAGGAATAAAAAAACGAATGATGTAACTTCCTTAAAT | |
| | | ATAAAATTGTTAAATTAAGTAACCAATTTATATTGCTTAT | |
| | | AATAGAGTTACCATAGGGATAACAGCGTAAT | |
| NC_00 | Sepioteuthis | GGGACGAGAAGACCCTATTGAGCTTGTAGTTTTTATATAA | 286 |
| 7894 | lessoniana | CTATATTATTAATTTATATAGTTATTTATAAACTTTAGTT | |
| | | GGGGTGACTAAGGAATAATATAAGTAACTTCCTTAGAGTA | |
| | | AAGATTGTTAAATAAAGTAACCAAATTTAATATTGCTTAT | |
| | | AATATAGTTACCATAGGGATAACAGCGTAAT | |
| NC_00 | Todarodes | GGGACGAGAAGACCCTAATGAGCTTATAATTTTATTATTG | 287 |
| 6354 | pacificus | TGATTTAATATATTTAAATGATAATATATAAATTTTAATT | |
| | | GGGGTGATTAAGGAATAATTTATAAGTAGTAACTTCCTTA | |
| | | GATTAAATATATATTGATTAAGTAACCAATAAATATATTG | |
| | | CTTATAATATAGTTACCATAGGGATAACAGCGTAAT | |
| NC_00 | Octopus | GGGACGAAAAGACCCTATTGAGCTTTTGTTATTATATAGA | 288 |
| 6353 | vulgaris | TATATATATGTATATATATATGATTTATTTGTAAATAACT | |
| | | TTGGTTGGGGTGATCAAGGAATAAAAATTAATTTAAAGTG | |
| | | TTTTATTAACTTCCTTAATTTTAAATTTAGTTGAAGAATA | |
| | | AACCAAGTTTTTTGCTTAGAAGATAAGTTACCATAGGGAT | |
| | | AACAGCGTAAT | |
| NC_00 | Heterololigo | GGACGAGAAGACCCTATATTTAATTCTTATTATTTTTTTA | 289 |
| 2507 | bleekeri | GATTTATTTTAATTGGGGTGATTAAGGAATAAAGTATATT | |
| | | AGTTAGTAACTTCCTTAGTAGGATATATTGTTGAATAAAG | |
| | | TAACCGATATTACGTATAGAACCGTATATTGCTTATAATA | |
| | | TAGTTACCATAGGGATAACAGCGTAAT | |
| MT712 | Octopus | GGGACGAAAAGACCCTATTGAGCTTTTGTTATTATATAGA | 290 |
| 046 | sinensis | TATATATATATATATATATATGATTTATTTGTAAATAACT | |
| | | TTGGTTGGGGTGATCAAGGAATAAAAATTAATTTAAAGTG | |
| | | TTTTATTAACTTCCTTAATTTTAAATTTAGTTGAAGAATA | |
| | | AACCAAGTTTTTTGCTTAGAAGATAAGTTACCATAGGGAT | |
| | | AACAGCGTAAT | |
| MT025 | Octopus | GGGACGAAAAGACCCTATTGAGCTTTTGTTGTTATATAGA | 291 |
| 991 | americanus | TATATATAAATATATAATTTATTTGTAAATAACTTTGGTT | |
| | | GGGGTGATCAAGGAATAAAATTAATTTAAAGTGTTATATT | |
| | | AACTTCCTTAATTTTGAATTTAGTTGAAGAATAAACCAAG | |
| | | TTTTTTGCTTAGAAGATAAGTTACCATAGGGATAACAGCG | |
| | | TAAT | |
| MG54 | Narrowteuthis | GGGACGAGAAGACCCTATTGAGCTTATAATTTTACTGTTG | 292 |
| 8981 | nesisi | TTTATAAGTATTTATTTGATGATGGATAAATTTTAATTGG | |
| | | GGTGATTGAGGAATAAAATATATTAAATAGTAACTTCCTT | |
| | | AGGTTAAATGTTGTTGGATAAAGTAACCAATATATTATTG | |
| | | CTTATAATATAGTTACCATAGGGATAACAGCGTAAT | |
| NC_02 | Ommastrephes | GGGACGAGAAGACCCTAATGAGCTTATAATTTTATTATCA | 293 |
| 0348 | bartramii | TTATTTAAAACATTTAGGTGGTGGTATATAAATTTTAATT | |
| | | GGGGTGATTAAGGAATAAATTGTGTATAGTAACTTCCTTA | |
| | | AATTAGGTATTTGTAGATAAAGTAACCAATGTAATATTGC | |
| | | TTATAGTATAGTTACCATAGGGATAACAGCGTAAT | |
| NC_01 | Sepiella | GGGACGAGAAGACCCTAATGAGCTTAAATTTTATATTATT | 294 |
| 7749 | japonica | GTTATATATGTTTTATAATTAATTGGAAATTTTAATTGGG | |
| | | GTGATTAAGGAATAATATTAAGATAAATAACTTCCTTATA | |
| | | TAATAATAAATTGTTGAATTAAGTAACCAATATTATTGCT | |
| | | TTTAATAAAGTTACCATAGGGATAACAGCGTAAT | |
| NC_01 | Uroteuthis | GGGACGAGAAGACCCTATTGAGCTTATAATTTTATTAATA | 295 |
| 7746 | (Photololigo) | TTTTATAAATATTTATGTTGTGTTATATAAATTTTAATTG | |
| | edulis | GGGTGATTAAGGAATAATTTTATTGATTTTATAGTGTAAC | |
| | | TTCCTTAGAAAACATATTGTTAAATTAAGTAACCGATTTA | |
| | | TATATAGGTTTATATTGGGTATTGCTTATAATATAGTTAC | |
| | | CATAGGGATAACAGCGTAAT | |
| NC_01 | Doryteuthis | GGGACGAGAAGACCCTATTGAGCTTGTAATTTTATTATTA | 296 |
| 2840 | (Amerigo) | TTAGTATTATTTATTCTTATTGGTAGTATATAAATTTTAA | |
| | opalescens | TTGGGGTGATTAAGGAATAAGGTTTGTAGTTTTAGTAACT | |
| | | TCCTTATTAATAATGATTGTTAAATAAAGTAACCAATATT | |
| | | ATTGCTTATACTATAGTTACCATAGGGATAACAGCGTAAT | |
| NC_01 | Architeuthis | GGGACGAGAAGACCCTATTGAGCTTATAATTTTATTGTTG | 297 |
| 1581 | dux | TTTTGTAATTATTTATGTGATGATAGATAAATTTTAATTG | |
| | | GGGTGATTGAGGAATAAGGTAATAGTAGTAACTTCCTTAG | |
| | | AAATAAATGTTGTTGGGTGAAGTAACCAATATATTATTGC | |
| | | TTATAATATAGTTACCATAGGGATAACAGCGTAAT | |
| NC_00 | Dosidicus | GGGACGAGAAGACCCTAATGAGCTTGTAATTTTATTATTG | 298 |
| 9734 | gigas | TTATTTAAAATATTTAGATAGCAATATATAAATTTTAATT | |
| | | GGGGTGATTAAGGAATAAGTTGAATATAGTAACTTCCTTA | |
| | | AATTGAATATTTATAGATAAAGTAACCAATATAATATTGC | |
| | | TTATAATATAGTTACCATAGGGATAACAGCGTAAT | |
| NC_00 | Sepiae | GGGACGAGAAGACCCTATTGAGCTTAAATTTAAAAGTACT | 299 |
| 9690 | sculenta | ATAGTAATTTTTTTAATATTTTGATAAATTTTAATTGGGG | |
| | | TGATTAAGGAATAAAAGGATAATAAATTGGTAACTTCCTT | |
| | | AAACTTAATACTATTAGGTTAAGTAACCAATAATATTGCT | |
| | | TATAATAAAGTTACCATAGGGATAACAGCGTAAT | |
| NC_00 | Amphioctopus | GGGACGAAAAGACCCTATTGAGCTTTATATTATTGTTTAA | 300 |
| 7896 | fangsiao | ATTTATTATGCTGTTTTTATTTAAAATAATTTTGGTTGGG | |
| | | GTGATCAAGGAATAAAATATTAAATTTATTAACTTCCTTA | |
| | | TTATATTTAAATATCTATATTAATAAACCAAACTTTTTGC | |
| | | TTAGAAGATAAGTTACCATAGGGATAACAGCGTAAT | |
| X7958 | Loligo | GGGACGAGAAGACCCTACTGAGCTTATAATTTTATTATTA | 301 |
| 5 | vulgaris | TTGTTATTTTTATTGGAGCAGTAATATATAAATTTTAATT | |
| | | GGGGTGATTAAGGAATAAATAAATATATTTAGTAACTTCC | |
| | | TTATAAAGAATATTGTTAGATGAAGTAACCAATAAAGTTA | |
| | | CATGATTATTGTAATAAATATTGCTTATAATATAGTTACC | |
| | | ATAGGGATAACAGSGTAAT | |
| NC_04 | Octopus | GGGACGAAAAGACCCTATTGAGCTTGTATTATTTAACAAA | 302 |
| 4093 | mimus | TTATATGATTGTTATTTGTTTGAATTAATTTTGATTGGGG | |
| | | TGATCAAGGAATAAATTTTATTTAGTTTAACTTCCTTAGT | |
| | | TAATTGTTTTATAGAAGAATAAACCAAAGTTTTTGCTTAG | |
| | | AAGATAAGTTACCATAGGGATAACAGCGTAAT | |
| NC_02 | Octopus | GGGACGAGAAGACCCTATTGAGCTTTATATTAGTTATAAT | 303 |
| 9747 | conispadiceus | AAATAATAAAAATATAATTTATTTGGTTAAATTAGTATTA | |
| | | TATTAATAATATTTATTATGTTTAATTTTGATTGGGGTGA | |
| | | TCAAGGAATAAAGTGGATATTATTTAACTTCCTTAAAATT | |
| | | TAATTTTTTTAGAATAATTAACCAATGATATTGCTTATAT | |
| | | GATAAGTTACCATAGGGATAACAGCGTAAT | |
| NC_02 | Octopus | GGGACGAAAAGACCCTATTGAGCTTATATTATTTATTAAA | 304 |
| 9723 | bimaculoides | TTATATAGTTATAGTTTATTATAAATTAATTTTGATTGGG | |
| | | GTGATCAAGGAATAAATTTTATTATTATATAACTTCCTTA | |
| | | GTTAAAATAGTTTTTGGAAAAATAAACCAAGTTTTTTGCT | |
| | | TAGAAGATAAGTTACCATAGGGATAACAGCGTAAT | |
| NC_02 | Uroteuthis | GGGACGAGAAGACCCTACTGAGCTTATAATTTTATTATCA | 305 |
| 8189 | (Photololigo) | CATATATTTATGTATAATGTGCTATATAAATTTTAATTGG | |
| | chinensis | GGTGATTAAGGAATAATATAGCTATTTTATAACTTCCTTA | |
| | | GATATTATATTGTTGAATTAAGTAACCGATTAATTAATAG | |
| | | AATTGTATTATGTATTGCTTATAATATAGTTACCATAGGG | |
| | | ATAACAGCGTAAT | |
| NC_02 | Uroteuthis | GGGACGAGAAGACCCTATTGAGCTTATAATTTTATTACTA | 306 |
| 7729 | (Photololigo) | TTATGGTTATTGAATTTATCATTGTAGTATATAAATTTTA | |
| | duvaucelii | ATTGGGGTGATTAAGGAATAAATTTTTATAATGTAACTTC | |
| | | CTTAGATTATTATATTGTTAAATTAAGTAACCGATGTGAT | |
| | | TAGCACAATGTGTGTTATGTATTGCTTATAATATAGTTAC | |
| | | CATAGGGATAACAGCGTAAT | |
| NC_02 | Illex | GGGACGAGAAGACCCTAATGAGCTTATAATTTTATTATTA | 307 |
| 6908 | argentinus | TTATTTAATATATTTAAATAATAATATATAAATTTTAATT | |
| | | GGGGTGATTAAGGAATAAAAGAATTATATAATAACTTCCT | |
| | | TAGATTAAATATATATATATATAAAGTAACCAATAATATA | |
| | | TTGCTTATAATATAGTTACCATAGGGATAACAGCGTAAT | |
| NC_02 | Sepia | GGGACGAGAAGACCCTATTGAGCTTGAATTTAATTGTATT | 308 |
| 2959 | aculeata | ATTTTATAATATTTTAATAAATTTTAATTGGGGTGATTAA | |
| | | GGAATAAAAAATTTTATATATTGTATAGTAACTTCCTTAA | |
| | | TATGATTGTACTGTTAGGTTAAGTAACCAATAATATTGCT | |
| | | TATAATAGAGTTACCATAGGGATAACAGCGTAAT | |
| NC_02 | Sepiella | GGGACGAGAAGACCCTAATGAGCTTAAATTTTATATTATT | 309 |
| 2693 | inermis | ATTAATATATAATTATAATAATTGGAAATTTTAATTGGGG | |
| | | TGATTAAGGAATAATATTAAAATAAATAACTTCCTTATAT | |
| | | ATTAATAAATTGTTGAATTAAGTAACCAATAATATTGCTT | |
| | | TTAATAAAGTTACCATAGGGATAACAGCGTAAT | |
| NC_02 | Sepia | GGGACGAGAAGACCCTATTGAGCTTAAATTTAATTACATA | 310 |
| 2468 | lycidas | ATTTAATTATGTTTTGATAAATTTTAATTGGGGTGATTAA | |
| | | GGAATAAAATTATTATATAGTAACTTCCTTAATAGTTAAT | |
| | | ACTGTTGGGTTAAATAACCAATAATATTGCATATAATAAA | |
| | | GTTACCATAGGGATAACAGCGTAAT | |
| NC_02 | Sepia | GGGACGAGAAGACCCTAATGAGCTTTTAATTTAATAGTAT | 311 |
| 2467 | latimanus | TTAATGTAAAATTGAATGTAAAGTTGTATAAATTTTAATT | |
| | | GGGGTGATTAAGGAATAAAATTATTTTAAATAATAACTTC | |
| | | CTTAAATGTAGTATTGTTAGATTAATTAACCAATATTATT | |
| | | GCTTATAATAGAGTTACCATAGGGATAACAGCGTAAT | |
| NC_02 | Sepia | GGGACGAGAAGACCCTAATGAGCTTAAATTTAATAGTGCT | 312 |
| 2466 | apama | AAAATTATAAGTTAATAGGATTATGAAAATTTTAATTGGG | |
| | | GTGATTAAGGGATAATTTATTAAATAATAACTTCCTTAAA | |
| | | TATAATATTGTTGAATTAATTAACCAATTTATATTGCTTA | |
| | | TAGTATAGTTACCATAGGGATAACAGCGTAAT | |
| NC_02 | Sepia | GGGACGAGAAGACCCTATTGAGCTTAAATTTAGTAATATT | 313 |
| 1146 | pharaonis | ATGTTTAGTAGTATTTAATAAATTTTAATTGGGGTGATTA | |
| | | AGGAATAAAATTATATATTGAATAATAACTTCCTTAATTG | |
| | | TGTAATACTGTTAGGTTAAGTAACCAATAATATTGCTTAT | |
| | | AATAAAGTTACCATAGGGATAACAGCGTAAT | |
| NC_02 | Loliolus | GGGACGAGAAGACCCTATTGAGCTTATAATTTTTGTTAGT | 314 |
| 8034 | (Nipponol | GCTTTTATTAATATTTATAATTGTATTATATAAATTTTAA | |
| | oligo) | TTGGGGTGATTAAGGAATAACTGATTGGAAAGTAACTTCC | |
| | beka | TTAGATGTGTGTATTGTTAGATTAAGTAACCGATTAATTA | |
| | | ATATATTGTATTAGATATTGCTTATAATATAGTTACCATA | |
| | | GGGATAACAGCGTAAT | |
| AF110 | Alloteuthis | GGGACGAGAAGACCCTATTGAGCTTATAATTTTATTGTTA | 315 |
| 072 | subulata | TTATATTTATATTAATATAGTAATTTATAAATTTTAATTG | |
| | | GGGTGATTAAGGAATAAAGATAAATAATTAAAGTAACTTC | |
| | | CTTAAAATAAAGTATTGTTAAATAAAGTAACCGATAAATT | |
| | | AGTATTAAAATAATACTATATATTGCTTATATTATAGTTA | |
| | | CCATAGGGATAACAGCGTAAT | |
| AB270 | Nototodarus | GGGACGAGAAGACCCTAATGAGCTTATAATTTTATTATTA | 316 |
| 952 | hawaiiensis | TGATTTGATATATTTGAATGATAATATATAAATTTTAATT | |
| | | GGGGTGATTAAGGAATAAGGTTTTATATTAATAACTTCCT | |
| | | TAAATTAAATATATATGTATTAAGTAACCAATAAATATAT | |
| | | TGCTTATAATATAGTTACCATAGGGATAACAGCGTAAT | |
| X7957 | Sepia | GGGAAGAGAAGACCCTATTGAGCTTTAATTTAAAAGACTA | 317 |
| 8 | orbignyana | TTAAAAAATTATAGTTAATAAAAATTTTAATTGGGGTGAT | |
| | | TAAGGAATAAAAAAGGCAACTTCCTTAAAATTAAAAGTTG | |
| | | TTAGGTTAAGTAACCAATAATATTGCTTTTATAAAGTTAC | |
| | | CATAGGGATAACAGCGTAAT | |
| X7958 | Sepia | RGGACGAGAAGACCCTATGAGCTTAAATTTATTAATTTTT | 318 |
| 6 | papuensis | ATTAGAATTTATAAAAATTTATAAAATTTTAATTGGGGTG | |
| | | ATTAAGGAATAAGTGTTTAAATAGTAACTTCCTTAGATAT | |
| | | AATTTTGTTAGATTAATTAACCAATTAATATTGCTTATAA | |
| | | AATAGTTACCGTAGGGATAACAGCGTAAT | |
| X7957 | Rossia | GGGACGAGAAGACCCTATTGAGCTTATAATTTTATTTTGT | 319 |
| 7 | macrosoma | TGTTTATTGAAATAGTAGGGTATAAATTTTAATTGGGGTG | |
| | | ATTAAGGAATAAGGTGATAGATAACTTCCTTAGGTAATAA | |
| | | TGTAGATGTATGAAGTAACCAATAATGCATTGCTTATATT | |
| | | ATAGTTACCATAGGGATAACAGCGTAAT | |
| AF110 | Lolliguncula | GGGACGAGAAGACCCTATTGAGCTTGTAATTTTATTAGTT | 320 |
| 084 | (Lolliguncula) | ACCAGTTAATATTATATAAATATGGTAACTTATAAATTTT | |
| | brevis | AATTGGGGTGATTAAGGAATAAATGTTTAGTTAGTAACTT | |
| | | CCTTAGATTAATAAATAAATTGTTAAATGAAGTAACCAAT | |
| | | AAAATTTAATGTATAAATATTGCTTATAATATAGTTACCA | |
| | | TAGGGATAACAGCGTAAT | |
| EU735 | Lolliguncula | GGGACGAGAAGACCCTATTGAGCTTATAATTTTATTAATT | 321 |
| 243 | (Loliolopsis) | ATCAATATTTATATATGTATTGATTTGGTAATTTATAAAT | |
| | diomedeae | TTTAATTGGGGTGATTAAGGAATAAATTTTATTTATAGTA | |
| | | ACTTCCTTAGATAAGTTAATAAATGATTGTTAAATGAAGT | |
| | | AACCAATAAAAATTAGGTGATATTGCTTATAATATAGTTA | |
| | | CCATAGGGATAACAGCGTAAT | |
| AF110 | Afrololigo | GGGACGAGAAGACCCTATTGAGCTTATAATTTTGTTATTA | 322 |
| 085 | mercatoris | TTATATTTATATTAATATAGTAATTTATAAATTTTAATTG | |
| | | GGGTGATTAAGGAATAAAGATAAATATGTAAGTAACTTCC | |
| | | TTAATAAATTATATTGTTAAATGAAGTAACCAATATAATA | |
| | | TTAGTAATAATATATATTGCTTATAGTATAGTTACCATAG | |
| | | GGATAACAGCGTAAT | |
| NC_01 | Octopus | GGGACGAGAAGACCCTATTGAGCTTTATTTATTAATATTT | 323 |
| 5896 | minor | AAATATAAATATATAATTTATATTATGAAAATTATGTTGT | |
| | | ATATATATATATATTTAATTGGATTTAATTTTGATTGGGG | |
| | | TGATCAAGGAATAAAGATAAAGTAACTTCCTTAAAATTAA | |
| | | TGATTGTTTTTATAGAGTAATAAACCAAGTTTTTTGCTTA | |
| | | TAAGATAAGTTACCATAGGGGTAACAGCGTAAT | |
| NC_02 | Octopus | GGGACGAAAAGACCCTATTGAGCTTATATTATTTATTAAA | 324 |
| 8547 | bimaculatus | TTATAGAGTTATAATTTATTATAAATTAATTTTGGTTGGG | |
| | | GTGATCAAGGAATAAGTTTAATGATTATATAACTTCCTTA | |
| | | ACTGAACTATTTTTTTGGAAAAATAAACCAAGTTTTTTTG | |
| | | CTTAGAAGATAAGTTACCATAGGGATAACAGCGTAAT | |
| NC_03 | Octopus | GGGACGAGAAGACCCTATTGAGCTTTATTTATTAATATTT | 325 |
| 8213 | variabilis | AAATATAAATATATAATTTATATTATGAAAATTATGTTGT | |
| | | ATATATATATATATTTAATTGGATTTAATTTTGATTGGGG | |
| | | TGATCAAGGAATAAAGATAAAGTAACTTCCTTAAAATTAA | |
| | | TGATTGTTTTTATAGAGTAATAAACCAAGTTTTTTGCTTA | |
| | | TAAGATAAGTTACCATAGGGGTAACAGCGTAAT | |
| AB191 | Octopus | GGGACGAAAAGACCCTATTGAGCTTTATATATTAATATAT | 326 |
| 115 | cyanea | AAATTTAATTGGTGATTTATAAAAAGTTTATTAATATTAA | |
| | | TTTTGGTTGGGGTGATCAAGGAATAAAAGGTAAATTGTAT | |
| | | TTTAGATAACTTCCTTAGAAAAATAATTGATGGAGTAATA | |
| | | AACCAAGATTTTTGCTTATAAGATAAGTTACCATAGGGAT | |
| | | AACAGCGTAAT | |
| AB191 | Callistoctopus | GGGACGAGAAGACCCTATTGAGCTTGGTTATTAATAATTA | 327 |
| 114 | ornatus | AATATAAAATAAATTGTTTTTATGGATTTATTTAGTATGA | |
| | | TAGTATATAAATATATTTAGTTAAATTAATTTTGATTGGG | |
| | | GTGATCAAGGAATAAAATATTTAAAAAGTAACTTCCTTAA | |
| | | TTTTTATTAGAGAATTTTAATTGAGTAATAAACCAAAGTT | |
| | | TTTGCTTATAAGATAAGTTACCATAGGGGTAACAGCGTAA | |
| | | T | |
| MG99 | Enteroctopus | GGGACGAGAAGACCCTATTGAGCTTTATATTAGTTATAAT | 328 |
| 9649 | megalocyathus | AAATAATAAAATTTTATTTATTTGGTTAAATAATATTATG | |
| | | TTTATTATGTTTAATTTTGATTGGGGTGATCAAGGAATAA | |
| | | GATAAGTATAATTTAACTTCCTTAAAAATAATTTTTTTAT | |
| | | AGAATAATTAACCAATAATATTGCTTATATGATAAGTTAC | |
| | | CATAGGGATAACAGCGTAAT | |
| AB191 | Enteroctopus | GGGACGAGAAGACCCTATTGAGCTTTATATTAGTTATAAT | 329 |
| 107 | dofleini | AAATAATAAAAATATAATTTATTTGGTTAAATTAGTATTA | |
| | | TATTAATAATATTTATTATGTTTAATTTTGATTGGGGTGA | |
| | | TCAAGGAATAAAGTGGATATTATTTAACTTCCTTAAAATT | |
| | | TAATTTTTTTAGAATAATTAACCAATGATATTGCTTATAT | |
| | | GATAAGTTACCATAGGGATAACAGCGTAAT | |
| GQ226 | Sasakiopus | GGGACGAGAAGACCCTATTGAGCTTTATATTAGTTATAAT | 330 |
| 031 | salebrosus | AAATGGTAAAATATAATTTATTACGTTTATTGTGTTTAAT | |
| | | TTTGATTGGGGTGATCAAGGAATAAAATAAATATAATTTA | |
| | | ACTTCCTTAGAAAAATTATTTTTTAGAATAATTAACCAAT | |
| | | GATATTGCTTATATGATAAGTTACCATAGGGATAACAGCG | |
| | | TAAT | |
| AY545 | Octopus | GGGACGAGAAGACCCTATTGAGCTTAATATTAATATTTAA | 331 |
| 105 | berrima | ATATAAAAATTTTTAGTTTTTATTTTATATGAATGTTTAT | |
| | | TGAATGTAATTTTGATTGGGGTGATCAAGGAATAAAAGTA | |
| | | AATATTAACTTCCTTAAGTTGTTGATTTTATTGAGTAATA | |
| | | AACCAAATTTTTTGCTTATAAGATAAGTTACCATAGGGGT | |
| | | AACAGCGTAAT | |
| NC_03 | Amphioctopus | GGGACGAAAAGACCCTATTGAGCTTTATATTATATTTTAA | 332 |
| 6351 | marginatus | ATATAATTTGTTTTGTATTTATTTAAATATAATTTTGGTT | |
| | | GGGGTGATCAAGGAATAAAGTATATTTGTATTAACTTCCT | |
| | | TAGATATTATATATAAATAAATTAATAAACCAAATTTATT | |
| | | GCTTAGAAGATAAGTTACCATAGGGATAACAGCGTAAT | |
| AJ311 | Octopus | GGGACGAGAAGACCCTATTGAGCTTGTATATTAATATTTA | 333 |
| 110 | maorum | AATGTTAGATGGTTTATTTATTATATTGTTAATAAATGAA | |
| | | TAGATTTTTGTTGATTAATAAGTATGGATGTTTAATTTAA | |
| | | TTAATTTTGATTGGGGTGATCAAGGAATAAAAGAAAAGAA | |
| | | ATTTAATAACTTCCTTAAGTTTTGAAATGTTTATTGAATA | |
| | | ATAACCAAATTTTTTTGCTTATAAGATAAGTTACCATAGG | |
| | | GGTAACAGCGTAAT | |
| MK450 | Octopus | GGGACGAGAAGACCCTATTGAGCTTAGAATTATTATTTAA | 334 |
| 541 | fitchi | TTAATGTGATAATTTATTTATTAGTTTATTGATATTTATT | |
| | | TATGTAATTTTGATTGGGGTGATCAAGGAATAAAAGTTTA | |
| | | TAATAACTTCCTTAGGATTTTGTAATTTATAGATTAATAA | |
| | | ACCATAATTTTGCTTAGAAGATAAGTTACCATAGGGATAA | |
| | | CAGCGTAAT | |
| NC_04 | Amphioctopus | GGGACGAAAAGACCCTATTGAGCTTTATATTATATTTTAA | 335 |
| 9899 | neglectus | ATTTGATTTTATATTTATTTACATATAATTTTGGTTGGGG | |
| | | TGATCAAGGAATAAAATGTATTTATATTAACTTCCTTAAA | |
| | | TGGTGATATATATAAATGAATAAACCAAATTTTTTGCTTA | |
| | | GAAGATAAGTTACCATAGGGATAACAGCGTAAT | |
| NC_02 | Loliolus | GGGACGAGAAGACCCTATTGAGCTTATAATTTTATTAATA | 336 |
| 6724 | (Nipponol | TTTTATTGTTATTTATAATTATATTATATAAATTTTAATT | |
| | oligo) uyii | GGGGTGATTGAGGAATAATTAATTAAATAGTAACTTCCTT | |
| | | AGGTATTTATATTGTTGAATTAAGTAACCGATTAATTAAT | |
| | | ATGTTTTATACAAGTATTGCTTATAATATAGTTACCATAG | |
| | | GGATAACAGCGTAAT | |
| NC_03 | Loliolus | GGGACGAGAAGACCCTATTGAGCTTATAATTTTATTGATA | 337 |
| 0208 | (Nipponol | TTTTTATTATTATTTATAATTATATTATATAAATTTTAAT | |
| | oligo) | TGGGGTGATTAAGGAATAATTAGTTAAAAAATAACTTCCT | |
| | japonica | TAAGTGTAATATATTGTTGAATTAAGTAACCGATTAATTA | |
| | | ATATTTTTATATTAGTATTGCTTATAATATAGTTACCATA | |
| | | GGGATAACAGCGTAAT | |
| NC_01 | Bathyteuthis | GGGACGAGAAGACCCTATTGAGCTTGTAATTTTATTATTA | 338 |
| 6423 | abyssicola | TTATATTAGTAGTATTGATATAGTAATATATAATTTTAGT | |
| | | TGGGGTGATTAAGGAATAAATTGTATAGTAACTTCCTTAG | |
| | | GTTATATATTTTATTATGAAGTAACCAATATAGTATTGCT | |
| | | TATATTATAGTTACCATAGGGATAACAGCGTAAT | |
| NC_01 | Semirossia | GGGACGAGAAGACCCTACTGAGCTTGTAATTTTATTTTAT | 339 |
| 6425 | patagonica | TATTTATTGTAATAGTAATGTATAAATTTTAATTGGGGTG | |
| | | ATTAAGGAATAAATATAATATTAAAGTAACTTCCTTAAAT | |
| | | AAAAATATAGATATATAAAGTAACTAATAATTATATTACT | |
| | | TATATTATAGTTACCATAGGGATAACAGCGTAAT | |
| NC_02 | Cistopus | GGGACGAAAAGACCCTATTGAGCTTATATTAATATTTATA | 340 |
| 3257 | taiwanicus | GTATTATAAGTATATATATATATATATATATATATATATA | |
| | | TATATATATATATATAGAGTATATGTTATAAATATATTAA | |
| | | TTTTGGTTGGGGTGATCAAGGAATAAAGTATAGAAAAATT | |
| | | TTTATATAACTTCCTTAATTTTTAATTTTATTGATTAATA | |
| | | AACCAATATTTTTGCTTATATGATAAGTTACCATAGGGAT | |
| | | AACAGCGTAAT | |
| NC_01 | Sthenoteuthis | GGGACGAGAAGACCCTAATGAGCTTATAATTTTATTATTG | 341 |
| 0636 | oualaniensis | TTGTGCAAAACATTTGGTCAATAATATATAAATTTTAATT | |
| | | GGGGTGATTGAGGAATAAATTAGATAAAGTAACTTCCTTA | |
| | | AATTAAATATTTATAGATAAAGTAACCAATATAATATTGC | |
| | | TTATAATATAGTTACCATAGGGATAACAGCGTAAT | |
| NC_00 | Wataseni | GGGACGAGAAGACCCTAATGAGCTTTAATTTTATTAATTA | 342 |
| 7893 | ascintillans | TTTATTTTAACAAATTTGTAGTTATATAAATTTTAATTGG | |
| | | GGTGATTAAGGAGTAAATTTAAGTAACCTCCTTATTTTTA | |
| | | TTACTGATAAGTAAAATAACCAATTTTGTTGCTTATACTA | |
| | | GAGTTACCATAGGGATAACAGCGTAAT | |
| EU735 | Gonatopsis | GGGACGAGAAGACCCTATTGAGCTTATAATTTTATCATTA | 343 |
| 265 | okutanii | TTAGACTTATTATTAGTGTAGTAGTGTATAAATTTTAATT | |
| | | GGGGTGATTAAGGAATAAATTTAATTTAGTAACTTCCTTA | |
| | | AGTTTTATATTGTTAAATAAAGTAACCAATAAGTTATTGC | |
| | | TTATAATATAGTTACCATAGGGATAACAGCGTAAT | |
| AY293 | Uroteuthis | GGGACGAGAAGACCCTATTGAGCTTATAATTTTATTAGTG | 344 |
| 656 | (Aestuariolus) | CTTAGTTTAAATACAAATGCATATATATAAATTTTAATTG | |
| | noctiluca | GGGTGATTAAGGAATAAAAATAATAAATTATAAGTAACTT | |
| | | CCTTAGAAGTATTAGTATTGATAAATGAAGTAACCAATAA | |
| | | ATAAACATATATTGCTTATAACATAGTTACCATAGGGATA | |
| | | ACAGCGTAAT | |
| AB675 | Sepioteuthis | GGGACGAGAAGACCCTAATGAGCTTATAATTGTATTATTA | 345 |
| 085 | australis | TTATAATTAGATGTTTATAATAGTTTATAAATTTTAATTG | |
| | | GGGTGATTAAGGAATATATGTACACAACTTCCTTAAATAT | |
| | | AATATTGTTGGATGGAGTAACCAATAGATTATTGCTTATA | |
| | | ATAAAGTTACCATAGGGATAACAGCGTAAT | |
| AF110 | Sepioteut | GGGACGAGAAGACCCTATTGAGCTTGAATTTTATAATAAT | 346 |
| 090 | hissepioidea | TATGTATATTAATAGCAGTAATTATATATAAATTTTAATT | |
| | | GGGGTGATTGAGGAATAATTTTTATGAAGTAACTTCCTTA | |
| | | AAATAAATATTGTTAAATTAAGTACCCGATTAATAGTATA | |
| | | TTGCTTGTAATATAGTTACCATAGGGATAACAGCGTAAT | |
| AJ311 | Amphioctopus | GCGGTAWTATAACTGTACTAAGGTAGCATAATAATTTGCC | 347 |
| 108 | kagoshimensis | TTATAAATTGGGGCTAGAATGAATGGTTTGACGAAAATTA | |
| | | TACTGTCTCTATTTTATTCATTAGAAGTTAATTTTTATAG | |
| | | TGAGAAAGCTTGGATGATTTAAAGGGACGAAAAGACCCTA | |
| | | TTGAGCTTTATATTATATTTTAAATATAATTTTTTGTGTT | |
| | | TATATAAATATAATTTTGGTTGGGGTGATCAAGGAATAAG | |
| | | ATATATCTATGTTAACTTCCTTAAATATATATATAAATTA | |
| | | ATAAACCAAATTTATTGCTTRGAAGATAAGTTACCATAGG | |
| | | GATAACAGCGTAAT | |
| LC121 | Amphioctopus | GGGACGAAAAGACCCTATTGAGCTTTATATTATATTTTAA | 348 |
| 044 | membranaceus | ATTTGATTTTTTATTTATTTAATTATATATAATTTTGGTT | |
| | | GGGGTGATCAAGGAATAAAATGTATTTTATGTTAACTTCC | |
| | | TTAAATGGTAATATTTATAAATGAATAAACCAAATTTTTT | |
| | | GCTTAGAAGATAAGTTACCATAGGGATAACAGCGTAAT | |
| LC121 | Amphioctopus | GGGACGAAAAGACCCTATTGAGCTTTATATTATTATTTAA | 349 |
| 036 | exannulatus | ATTTATTATATTAATTTTTATTTTAAATAATTTTGGTTGG | |
| | | GGTGATCAAGGAATAAAATATTAAATTTATTAACTTCCTT | |
| | | ATATATTTAAATATATATATATTAATAAACCAAATTTTTT | |
| | | GCTTAAAAGATAAGTTACCATAGGGATAACAGCGTAAT | |
| LC121 | Amphioctopus | GGGACGAAAAGACCCTATTGAGCTTTATATTATTATTTAA | 350 |
| 047 | rex | ATTTATTATATTAATTTTTATTTTAAATAATTTTGGTTGG | |
| | | GGTGATCAAGGAATAAAATATTAAATTTATTAACTTCCTT | |
| | | ATATATTTAAATATATATATATATTAATAAACCAAATTTT | |
| | | TTGCTTAAAAGATAAGTTACCATAGGGATAACAGCGTAAT | |
| AB192 | Sepia | GGGACGAGAAGACCCTATTGAGCTTTAAATTTGGAAATAT | 351 |
| 324 | peterseni | TATATTTATTAAATATAATGNTTGAAAAATTTTAATTGGG | |
| | | GTGATTAAGGAATAAAAGAAATAAATATATTAAAATTTAA | |
| | | CTTCCTTAAATAATATATTGTTAAATAAAGTAACCAATAA | |
| | | TATTGCTTATATAAAGTTACCATAGGGATAACAGCGTAAT | |
| AB058 | Varuna | TTGAAGTGACGATAAGACCCTATAAAACTTTATATAAACA | 352 |
| 620 | litterata | TATTATTTGATTGAATTTAAAATAAAAATTTAATAATAAA | |
| | | ATTTATTTTGTTGGGGCGATAAAAGTAAAATTTTTATTAA | |
| | | CTGCTTTAAGTTTTGAATACATTTATGTTTGATTAAAATT | |
| | | TTTAGATGATCCTAAATAAAGATTAAAAGTTTAAGTTACT | |
| | | TTAGGGATAACAGCGTTAT | |
| AF107 | Hemisquil | GGGGGACGATAAGACCCTATAAAGCTTTATGATAAGGCCT | 353 |
| 616 | laensigera | TTTTGTAAAGAATTTCTATTGTGAAATTACTAGAAGGTTT | |
| | | ATATCATTTTACTGGGGCGGTAAAAGTATATAATTGAATA | |
| | | ACTGCTTTTATTATTGAAAACAATTATAATTGAAATACAA | |
| | | AATGATCCATTATTAATGATTATAAAAACAAGTTACTTTA | |
| | | GGGATAACAGCGTAAT | |
| AF107 | Gonodact | GGAGGACGATAAGACCCTATAAAGCTTTATAATATATTTT | 354 |
| 615 | ylussmithii | CTGTTATATAAATTGTTAATTGTTAACTTATTAAAGAAAT | |
| | | AATATTATTTTACTGGGGCGGTAAAAGTATATAATTTAGG | |
| | | GTAACTGCTTTTGTTATTATAAACAATGATAATTGAATGA | |
| | | TATATTGATCCATTATTAGTGAGCATAAGGACAAGTTACT | |
| | | TTAGGGATAACAGCGTAAT | |
| AF107 | Pullosquilla | AAGGGACGATAAGACCCTATAAAGCTTTATAACATAATCT | 355 |
| 611 | thomassini | TTTTGCGCAAAATATCTAATGTAAAACATTAAAAGAATCA | |
| | | TGTTATTTTACTGGGGGGGTAAAAGTATATTAAAAATAAC | |
| | | TGCTTTTGTTTTATAAAACAATAATAATTGGTACACAGAT | |
| | | TGATCCATTAATAATGATCATAAAAACAAGTTACTTTAGG | |
| | | GATAACAGCGTAAT | |
| AF107 | Chorisquilla | GAGGGACGATAAGACCCTATAAAGCTTTATGATATTTTCT | 356 |
| 609 | trigibbosa | TTTTATATTAGTTAGTAAAATGTAGTTTATTAAAAGAAAA | |
| | | ATATTATTTTACTGGGGCGGTAGAAGTATATTTTTGATTA | |
| | | ACTGCTTCTGTTTTGTAAGTCAATTATAATTGAAATATGA | |
| | | ATTGATCCATTATTAATGATCATAAGAACAAGTTACTTTA | |
| | | GGGATAACAGCGTAAT | |
| MT335 | Chionoecetes | GGGGGACGATAAGACCCTATAAAGCTTTACATATAAGTAG | 357 |
| 860 | opilio | AGTTTACTTAATTAAAAAGTAAAAGTTTAATCTAATTAAT | |
| | | GTGTTTTGTTGGGGCGACATAAATATAATTTATATTAACT | |
| | | GTTTATAAATTAATACAATAATAAATGATTTTAATGTAGT | |
| | | TGATCCTTATTAAAGATTAAAAGACTAAGTTACTTTAGGG | |
| | | ATAACAGCGTTAT | |
| AB220 | Chionoecetes | AGGGGACGATAAGACCCTATAAAGCTTTACATATATTATA | 358 |
| 027 | opilio | AGTTTGATTAAATTAAAATATAAAAATCTAAATTATATAT | |
| | | TATGTTTTGTTGGGGCGACAAAAATATAAGTAATAATAAC | |
| | | TGTTTAAGAAAAAAACAATAATAAATGAATAATTAAATGA | |
| | | TCCTTGGTTAAAGATTAAAGATCAAGTTACTTTAGGGATA | |
| | | ACAGCGTTAT | |
| AB188 | Telmessus | AGGGGACGATAAGACCCTATAAAGCTTTACATATATTATA | 359 |
| 686 | acutidens | AATTTGATTAAATTAAAATCTAAAAATCTAAATTATATAT | |
| | | TATGTTTTGTTGGGGCGACAAAAATATAAGTAATAATAAC | |
| | | TGTTTAATAAAAAAACAATAATAAATGAATAATTAAATGA | |
| | | TCCTTATTTAAAGATTAAAGATCAAGTTACTTTAGGGATA | |
| | | ACAGCGTTAT | |
| AB236 | Lithodes | CAAAAAGACGATAAGACCCTATAAATCTTTACAATAAATA | 360 |
| 927 | aequispinus | TATTTTATATTTTAGCTTATAAGTGTATAAGAAATAAAAA | |
| | | TATAGGTTTGTTGCGCTGGGGCGGCGTAGATATATAAATA | |
| | | AACTGTCTATAATTTAAATACAATAATCATTGCTTAATAC | |
| | | AAATTGATCCTTAAATAGATTAAAAGATTAAGATACTTTA | |
| | | GGGATAACAGCGTTAT | |
| AB248 | Panulirus | GGGGGACGATAAGACCCTATAAATCTTGATAATAAGACTA | 361 |
| 090 | echinatus | ATAACTTCATATATTAGTTGTGAGTCTGAGGTTAATACTT | |
| | | TTATTATTTTGTTGGGGCGACAGAAGTATAATTTTAGTAA | |
| | | CTGCTTTGGCTTAATAGTTAATTATTTTTATAATGTTTAT | |
| | | TGGTTAAGATCCTTCTGTGAGGATGCCAGATTAAGTTACT | |
| | | TTAGGGATAACAGCGTAAT | |
| AF192 | Jasus | GGGGGACGATAAGACCCTATAAATCTTTATGNTAGTTAGT | 362 |
| 870 | paulensis | AATAACATTAAAAATTAATTTNACATTTTTTTGTTAGTAG | |
| | | TTTTAGCATTTTGCTGGGGCGGCAGGGGTATAATTTAATA | |
| | | ACTGCTCTAGAGTTGTAGTTAATTATGTTTAAATTATTGG | |
| | | TAGTTGATCCTTTAGTGGAGATTATAGATTAAGTTACTTT | |
| | | AGGGATAACAGCGCAAT | |
| AF192 | Jasus | GGGGGACGATAAGACCCTATAAATCTTTATGTTAGTTAGT | 363 |
| 869 | caveorum | AATAACATTAAAAATTAATTTTACATTTTTTTGTTAGTAG | |
| | | TTTTAGCATTTTGCTGGGGCGGCAGGGGTATAATTTAATA | |
| | | ACTGCTCTAGAGTTGTAGTTAATTATGTTTAAATTATTGG | |
| | | TAGCTGATCCTTTAGTGGAGATTATAGATTAAGTTACTTT | |
| | | AGGGATAACAGCGCAAT | |
| AF175 | Parastacus | AAGGGACGATAAGACCCTATAAAGCTTCACATTAAGTATT | 364 |
| 246 | pilimanus | TAAAATTTTAAGTGAGTAATAAAATTTTATTAGATGTAAT | |
| | | ATGTTTTGTTGGGGCGACAGGAATATAAGAGATTTAACTG | |
| | | TTCTAGATAAGTTAATCAAAAATACTTGATCTTTAAGGAC | |
| | | TTTTTATTAAAGTATTAGACTAAGTTACTTTAGGGATAAC | |
| | | AGCGTTAT | |
| MG55 | Parastacus | AAGGGACGATAAGACCCTATAAAGCTTCACATTAAGTCTT | 365 |
| 1495 | brasiliensis | TAAAATTTTAATTAAGTAATAAAATTTTATTAAATGCAAT | |
| | | ATGTTTTGTTGGGGCGACAGGAGTATAAGAAATTTAACTG | |
| | | CTCTAAATAATAAGTCAAAGATACTTGATCTTTGGGGACT | |
| | | TTTTATTAAAGTGTTAGATTAAGTTACTTTAGGGATAACA | |
| | | GCGTAAT | |
| AF175 | Parastacus | AAGGGACGATAAGACCCTATAAAGCTTTACATTAAGCTTT | 366 |
| 243 | defossus | TAAAATTTTAATTAAGTAATAAAATTTTATTAAATGCAAT | |
| | | ATGTTTTGTTGGGGCGACAGGAATATAAGAAATTTAACTG | |
| | | TTCTAAATAATAAATCAAAGATACTTGATCTTTGGGGACT | |
| | | TTTTGTTAAAGTGTTAGATTAAGTTACTTTAGGGATAACA | |
| | | GCGTTAT | |
| AF175 | Parastacus | AAGGGACGATAAGACCCTATAAAGCTTTACATTCAACTTT | 367 |
| 233 | nicoleti | TTAGGATTATTAATTAAGTAATAAAAGTTTGTTAGGTATA | |
| | | ATATGTTTTGTTGGGGCGACAGGAATAAAAGAGATTTAAC | |
| | | TGTTCTAAATAATAAATCAGAAATATTTGATTTTTAATGA | |
| | | TTTTTTATTAAAATATTAGATTAAGTTACTTTAGGGATAA | |
| | | CAGCGTTAT | |
| AF133 | Gonodactylus | GAGGGACGATAAGACCCTATAAAGCTTTATAGTAGTTCCT | 368 |
| 678 | graphurus | TTTTATTCAAATTATAAAATGTTAACCTTTTGAAAGATTA | |
| | | CTACTATTTTACTGGGGCGGTAGAAGTATATTTATGATTA | |
| | | ACTGCTTTTGTTTTGACAAACAATAATAATTGAATATTTA | |
| | | TGTGATCCATTATTAATGATCAAAAGTACAAGTTACTTTA | |
| | | GGGATAACAGCGTAAT | |
| AF192 | Jasus | GGGGGACGATAAGACCCTATAAATCTTTATACTAGTTAGT | 369 |
| 873 | lalandii | AATAACATTAAAGATTAATTTTACATTTTTTTGTTAGTAG | |
| | | TTTTAGTATTTTGCTGGGGCGGCAGGGGTATAATTTAATA | |
| | | ACTGCTCTAGAGTTGTAGTTAATTATGTTTAAATTGTTGG | |
| | | TAGTTGATCCTTTAGTGGAGATCATAGATTAAGTTACTTT | |
| | | AGGGATAACAGCGCAAT | |
| AF192 | Jasus | GGGGGACGATAAGACCCTATAAATCTTTATGTTAGTTAGT | 370 |
| 872 | paulensis | AATAACATTAAAAATTAATTTTACATTTTTTTGTTAGTAG | |
| | | TTTTAGCATTTTGCTGGGGCGGCAGGGGTATAATTTAATA | |
| | | ACTGCTCTAGAGTTGTAGTTAATTATGTTTAAATTATTGG | |
| | | TAGTTGATCCTTTAGTGGAGATTATAGATTAAGTTACTTT | |
| | | AGGGATAACAGCGCAAT | |
| AF425 | Lopholithodes | AAGACGATGAGACCCTATAAATCTTTACAATAAATATATT | 371 |
| 333 | mandtii | TTATATTTTAGCTTATAAGTATATAAAAAATAAAATATAG | |
| | | ACTTGTTGCGCTGGGGCGGCGTAGATATATAAATAAACTG | |
| | | TCTATAGTTTAAATACAATAATCATTGCTTAATACAAATT | |
| | | GATCCTTAAATAGATTAAAAGATTAAGATACTTTAGGGAT | |
| | | AACAGCGTTAT | |
| AF425 | Lithodes | CAAAAAGACGATAAGACCCTATAAATCTTTACAATAAATA | 372 |
| 331 | santolla | TATTTTATATTTTAACTTATAAGTGTATAAGAAATAAAAA | |
| | | TATAGATTTGTTTCGCTGGGGCGGCGTAGATATATAAATA | |
| | | AACTGTCTATAATTTAAATACAGTAATTATTGCTTAATAT | |
| | | AAATTGATCCTTAAATAGATTAAAAGATTAAGATACTTTA | |
| | | GGGATAACAGCGTTAT | |
| AF425 | Lithodes | CAAAAAGACGATAAGACCCTATAAATCTTTACAATAAATA | 373 |
| 330 | maja | TATTTTATATTTTAGCTTATAAGTGTATAAAAAATAAAAA | |
| | | TATAGATTTGTTGCGCTGGGGCGGCGTAGATATATAAATA | |
| | | AACTGTCTATAATTTAAATACAATAATCATTGTTTAATAT | |
| | | AGATTGATCCTTAAATAGATTAAAAGATTAAGATACTTTA | |
| | | GGGATAACAGCGTTAT | |
| AF337 | Jasus | GGGGGACGATAAGACCCTATAAATCTTTATACTAGTTAGT | 374 |
| 979 | edwardsii | AATAACATTAAAGATTAATTTTACATTTTTTTGTTAGTAG | |
| | | TTTTAGTATTTTGCTGGGGCGGCAGGGGTATAATTTAATA | |
| | | ACTGCTCTAGAGTTGTAGTTAATTATGTTTAAATTGTTGG | |
| | | TAGTTGATCCTTTAGTGGAGATTATAGATTAAGTTACTTT | |
| | | AGGGATAACAGCGCAAT | |
| AF337 | Panulirus | GGGGGACGATAAGACCCTATAAATCTTTATATTTCGGTCA | 375 |
| 976 | regius | GTAAATTTTAGAGATTAGTTATACATTTAGTGTTATTGAA | |
| | | CTCAAATATTTTGTTGGGGCGACAGGAGTATAATAAGTCA | |
| | | CTGCTTTGTTTATAAGTTAATTATGTTTATTTTGAAAACC | |
| | | AATATTGATCCTTTCTAGAGAAATCAGACTAAGTTACTTT | |
| | | AGGGATAACAGCGTAAT | |
| AF337 | Panulirus | GGGGGACGATAAGACCCTATAAATCTTGATAACCAAAGTA | 376 |
| 973 | pascuensis | ATAATTTTATGTAGTAGTCATAAGCTTAAATTTAATACTT | |
| | | TTGTTATTTTGTTGGGGCTACAGGGGGTATAATTAATAAC | |
| | | TGCGGTTGGGAAAAATAATTAATTATTTTTATTGTGTTGT | |
| | | GTTTAAGATCCTTCTTTGAAGATACAGGTCAAGTTACTTT | |
| | | AGGGATAACAGCGTAAT | |
| AF337 | Panulirus | GGGGGACGATAAGACCCTATAAATCTTTATATTTTAGTCG | 377 |
| 969 | laevicauda | ATAAGTTTTAGGAATTAGTTACACACTTAGTATCATTGGA | |
| | | CTAAAATATTTTGTTGGGGCGACAGAAGTATAATAAGTAA | |
| | | CTGCTTTGTTTATAAATTAATTATTTTTATTATGAGAGCT | |
| | | GATATAGATCCTCTTTAGAGAAATCAGATTAAGTTACTTT | |
| | | AGGGATAACAGCGTAAT | |
| AF337 | Panulirus | GGGGGACGATAAGACCCTATAAATCTTTATGTTTTGGTTA | 378 |
| 964 | gracilis | ATAAATTTTAGAAATTAGTTATATATTTAGTATTATTAGG | |
| | | CTAAAATATTTTGTTGGGGCGACAGGAGTATAATAAGTAA | |
| | | CTGCTTCGGTTTGTAAATTAATTATTTTTATATTGGAAAT | |
| | | GTAATATTGATCCTTTTTAGAGAAATCAGACTAAGTTACT | |
| | | TTAGGGATAACAGCGTAAT | |
| AF337 | Panulirus | GGGGGACGATAAGACCCTATAAATCTTGATAGAGAAGATT | 379 |
| 963 | guttatus | ATAACCTTAGAAAGTAGTTACAGTTCTCGGGTTAAGACAT | |
| | | TTTCTATTTTGTTGGGGCGACAGGAGTATAATTAGTAACT | |
| | | GCTTTGGGCTTAATATGTAATTATCGCTATTGTGGGTATT | |
| | | ATGAGAGATCCTTCTTTGAAGATGTCAGACTAAGTTACTT | |
| | | TAGGGATAACAGCGTAAT | |
| AF337 | Panulirus | GGGGGACGATAAGACCCTATAAATCTTTATGTTTTAACTA | 380 |
| 960 | inflatus | ATAAATTTTAGGGATTAGTTATATACCTAGTATTATTAGT | |
| | | TGAAATATTTTGTTGGGGCGACAGGAGTATAATAAGTAAC | |
| | | TGCTTTGTTTATAAAATTAATTGTTTTTATTGTGGAAATT | |
| | | GGTATGATCCTCTTTAGAGAAATCAGACTAAGTTACTTTA | |
| | | GGGATAACAGCGTAAT | |
| AF339 | Panulirus | GGGGGACGATAAGACCTATAAATCTTGATAACCAAAGTAA | 381 |
| 156 | femoristriga | TAATTTTTATGTAGTAGTCATAAGCTTTAAATTTAATACT | |
| | | TTTGTTATTTTGTTGGGGCGACAGGGGTATAATTAGTAAC | |
| | | TGGCTTTGAGTAAAGAATTAATTATTTTTATTGTGTTGTG | |
| | | TTTGAGATCCTTCTTTGAAGATATCAGATCAAGTTACTTT | |
| | | AGGGATAACAGGTAATC | |
| AY227 | Chionoecetes | GGGGGACGATAAGACCCTATAAAGCTTTACATATAAGTAG | 382 |
| 446 | bairdi | AGTTTACTTAATTAAAAAGTAAAAGTTTAATCTAATTAAT | |
| | | GTGTTTTGTTGGGGCGACATAAATATAATTTATATTAACT | |
| | | GTTTATAAATTAATACAATAATAAATGATTTTAATGTAGT | |
| | | TGATCCTTATTAAAGATTAAAAGACTAAGTTACTTTAGGG | |
| | | ATAACAGCGTTAT | |
| AF425 | Paralomis | AAAAGACGATAAGACCCTATAAATCTTTACAAGAAATATA | 383 |
| 339 | granulosa | TTTTATGTTTTAGCTTATAAGTAATATAAGAAATAAAAAT | |
| | | ATAAGTTTGTTGCGCTGGGGCGGCGTAGATATATAAATAA | |
| | | ACTGTCTATGATTTAAATACAGTAATTATTGCTTAATACA | |
| | | AATTGATCCTTAAATAGATTAAAAGATTAAGATACTTTAG | |
| | | GGATAACAGCGTTAT | |
| AF502 | Scyllarus | GGGGGACGATAAGACCCTATAAATCTTTATACTCGGCTTT | 384 |
| 949 | arctus | ATTGAGCAAAATACTAGAATAATTTTTGTTCGATAGGTTT | |
| | | GAGTATTTTGTTGGGGCGACAGGAGTATAAAATGTAACTG | |
| | | CTTTAAAATAATAGTTAATTGATGTTTAGTTAAGCGATCC | |
| | | TTTAATAAAGATGTTAGTTTAAGTTACTTTAGGGATAACA | |
| | | GCGTAAT | |
| AF502 | Palinurus | AGGGGACGATAAGACCCTATAAATCTTTATATTTGTCATA | 385 |
| 955 | elephas | ATGATATTAAGTATTATAATTGAAAATTTAGTTTTATTGG | |
| | | GATAGAATATTTTGTTGGGGCGACAGGAGTATAAAAAAGT | |
| | | AACTGCTTTGTAAGATGAGTTAATAATTTTTAATGTGTGA | |
| | | TATAGTTGATCCTTCAGTGAGGATTTCAGAATAAGTTACT | |
| | | TTAGGGATAACAGCGTAAT | |
| AJ784 | Episesarma | AAAAGACGATAAGACCCTATAAAACTTAATATAAGTTAAT | 386 |
| 020 | mederi | TGTTTAGTAGAATTTTTAATAATAAATATTTAATAATTAG | |
| | | TTTTTATTTTATTGGGGTGATAATGGTAAAATGATTATTA | |
| | | ACTGTTGATTATTTTATAACAAAAATTATTGAATAAATAT | |
| | | ATAATAAATGATCCTTGTTAAAGATTAAAAGTTTAAGTTA | |
| | | CTTTAGGGATAACAGCGTTAT | |
| NC_03 | Austropotamobius | GGGGGACGATAAGACCCTATAAAACTTTATATTTTGAAAT | 387 |
| 3504 | torrentium | AATAATTAATTTTGTGTGGGAGATTTATTTTTAAGTATTT | |
| | | TATTGGGGTGATAAGGATAAAATAATAAATAACTGTCTTT | |
| | | TTTTATTACAGTAATATTTGAGCAAATGAACCTAGTAAGG | |
| | | GAAAGAAGATTAAGTTACTTTAGGGATAACAGCGTAAT | |
| AM410 | Cycloachelous | AGGGGACGATAAGACCCTATAAAGCTTTATAAATTCAGGA | 388 |
| 525 | granulatus | ATTTTAGATATTTTATATATAAATCTATTTTTTGATAATT | |
| | | TATTTGGTTGGGGCGACAAGGGTATAATTATAAATAACTG | |
| | | CCATTAATGTTTATACATATATTTTTGATTTAAATTTGAT | |
| | | ATAGTAGTTGATCCTTTTTAAGGATTTAAAGATCAAGTTA | |
| | | CTTTAGGGATAACAGCGTAAT | |
| AY151 | Eriocheir | AAGGGACGATAAGACCCTATAAAGCTTAATATAAAATTTT | 389 |
| 824 | recta | ATTTAGTCAAATTTTACGTTATAAAGACTTTGTAAGTATG | |
| | | AGTTTATTTTATTGGGGCGATAAGAGTAAAATGATTGTTA | |
| | | ACTGCTTAATATTAAATACACTTATGAGTGATTAAATTTT | |
| | | TAGATGATCCTAATTAAAGATTAGAAGTTTAAGTTACTTT | |
| | | AGGGATAACAGCGTTAT | |
| AY351 | Cervimunida | AAGGGACGATAAGACCTTATAAATCTTTATATTAATTTGT | 390 |
| 244 | johni | TTTTTGTCTTTTAATAATGTAATGTTGATAATAAAATAAA | |
| | | GTATATTGTGTTGGGGTGATGAGAATATAAAATTAACTGT | |
| | | TCTAAATTAAACACAAATTTATTTGTATAAAATAATAGAT | |
| | | CCTTATTAAAGATTAAAAGGTTAAGTTACTTTAGGGATAA | |
| | | CAGCGTTAT | |
| DQ388 | Achelous | GGGGGACGATAAGACCCTATAAAGCTTTATAAAAGTAGTA | 391 |
| 058 | floridanus | AGATCACATATTTGAATATAAAAGCGATTATATAATCTTT | |
| | | TATTTTGTTGGGGCGACAGTAGTATAATTGAAAGTAACTG | |
| | | CTATTATGAAAACACATTGATTTATGATTATTTATTAATG | |
| | | GATCCCTTTTAAGGATTATAAAATCAAGTTACTTTAGGGA | |
| | | TAACAGCGTTAT | |
| DQ388 | Portunus | GAGGGACGATAAGACCCTATAAAGCTTTATAAATTTAAAG | 392 |
| 053 | sayi | ATTTTACATATTTTATCTATAATGGTAATCTCTGGAGAAT | |
| | | TTATTGGGTTGGGGCAACAAAAGTATAATTATCAATAACT | |
| | | GCTATAAAATAATACAATTATCTTTGTGTGGGTAAATTAG | |
| | | TTGATCCCTGAAAAGGATTAAAAGATCAAGTTACTTTAGG | |
| | | GATAACAGCGTAAT | |
| DQ388 | Portunus | GAGGGACGATAAGACCCTATAAAGCTTTATAAACTAAAAG | 393 |
| 054 | anceps | AATTCACAAATTTAGACATAAAGGTAATTTTCTAACTTCT | |
| | | TATTTTGTTGGGGCGACAGTAGATAACTAAAGTAACTGCT | |
| | | ATTATAGGAGTACAATGATTTTTGATTAGCCAGTAACAGA | |
| | | TCCCTACTAAGGATTATAAAACCAAGTTACTTTAGGGATA | |
| | | ACAGCGTTAT | |
| DQ377 | Palinurus | GGGGGACGATAAGACCCTATAAATCTTTATATTTATTATG | 394 |
| 978 | gilchristi | ATAACATTAAATATTATAATTGAAAATTTAGTTTTACTGG | |
| | | AATAAAATATTTTGTTGGGGCGACAGGAGTATAAAAAATA | |
| | | ACTGCTTTGTAATATGGGTTAATAATTTTTAATATGTAGT | |
| | | ATAGTTGATCCTTCAGTGAGGATTTTAGAATAAGTTACTT | |
| | | TAGGGATAACAGCGTAAT | |
| DQ377 | Palinurus | GGGGGACGATAAGACCCTATAAATCTTTATATTTATTATG | 395 |
| 977 | mauritanicus | ATAACATTAAATATTATAATTGAAAATTTAATTTTACTGT | |
| | | AATAGGATATTTTGTTGGGGCGACAGGAGTATAAAAAATA | |
| | | ACTGCTTTGTAAGATGGGTTAATAATTTTTAATGTGTAGT | |
| | | ATATTTGATCCTTCAGTGAGGATTTTAGAATAAGTTACTT | |
| | | TAGGGATAACAGCGTAAT | |
| DQ377 | Palinurus | GGGGGACGATAAGACCCTATAAATCTTTATATTTATTATG | 396 |
| 975 | charlestoni | ATAACATTAAATATTATAATTGAAAATTTAGTTTTACTGG | |
| | | AATAAAATATTTTGTTGGGGCGACAGGAGTATAAAAAATA | |
| | | ACTGCTTTATAAGATAGGTTAATAATTTTTAATATGTAGT | |
| | | ATAGTTGATCCTTCAGTGAGGATTTTAGAATAAGTTACTT | |
| | | TAGGGATAACAGCGTAAT | |
| AY947 | Pseudosquilla | AGTGACGATAAGACCCTATGAAAGCTTTATGAGTGTGCTT | 397 |
| 836 | ciliata | CTTTTATATGTAAAATGAATTTAATATTAACATATTAAAG | |
| | | AAATCACATTATTTTACTGGGGCGGTAAAAGTATAAATGA | |
| | | TTAACTGCTTTTGTTATGCAAAACAATGATAATTGGTATG | |
| | | TAAATTGATCCATTAATAATGATTATAAAAACAAGTTACT | |
| | | TTAGGGATAACAGCGTAAT | |
| AY351 | Pleuroncodes | AAGGGGCGATAAGACCCTATAAATCTTTATATTAAATTGT | 398 |
| 259 | monodon | TTTTAGTTTATTAATAATGTAATTTAAATAATAAGGCAAA | |
| | | ATATATTGTGTTGGGGTGATGGGAATATAAAAAATTAACT | |
| | | GTTCTGAAATAAAAACAAATTTATTTGATTTAATAATAGA | |
| | | TCCTTTTTAAAGATTAAAAGTTTAAGTTACTTTAGGGATA | |
| | | ACAGCGCTAT | |
| DQ388 | Portunus | AGGGGACGATAAGACCCTATAAAGCTTTATAAATCTATAA | 399 |
| 060 | ventralis | ATTATTACATATTTAAAATATAAAGGTAATCTTTTAGATA | |
| | | TTTATTTGGTTGGGGCGACAGTGGTATAAATTAATTAACT | |
| | | GCTAATAAAAGTTACAATAATTTTTGATTCTTAAGTTATA | |
| | | GATCCTCTCTAGGGATTAAAAAATCAAGTTACTTTAGGGA | |
| | | TAACAGCGTTAT | |
| DQ388 | Achelous | GGGGGACGATAAGACCCTATAAAGCTTTATAAATTTATAG | 400 |
| 061 | spinicarpus | ACTTTATATATTTAAAATAAAAATAATCTCTAAAACATTT | |
| | | ATTTGGTTGGGGCGACAAAAGTATAATTTCAAATAACTGC | |
| | | TATTAAATTAACAATAATTTTTGTTTATGAATAATTGATC | |
| | | CTTCTATGAGGAGTAAAAGTTCAAGTTACTTTAGGGATAA | |
| | | CAGCGTCAT | |
| DQ407 | Callinectes | GAGGGACGATAAGACCCTATAAAGCTTTATAAATTTATTA | 401 |
| 681 | toxotes | ATTTCGTTTATTTTTTATTATAATAATGATGATAAGAAAA | |
| | | TTTATTTGGTTGGGGCAACCGAAGTATAATTTTAAATAAC | |
| | | TGCTATAATTTAATACAATGATTTTTGTTTTATATAATAA | |
| | | TTGATCCTCTAAGAGATTAAAGACCAAGTTACTTTAGGGA | |
| | | TAACAGCGTAAT | |
| DQ407 | Callinectes | GAGGGACGATAAGACCCTATAAAGCTTTATAAATTTATTA | 402 |
| 680 | danae | ATTTCTTCTATTTTTTTATAATGAGGATGTTAAGAAGATT | |
| | | TATTTGGTTGGGGCAACCGAAGTATAATTAAAGATAACTG | |
| | | CTAATAGTTTATACAATGATTTTTGTTTTAATAATAATTG | |
| | | ATCCTTAAAAAGATTAAAGACCAAGTTACTTTAGGGATAA | |
| | | CAGCGTAAT | |
| DQ407 | Callinectes | GAGGGACGATAAGACCCTATAAAGCTTTATAAATTTATTG | 403 |
| 679 | ornatus | ATTTCGTCTATTTTTCTTATAATAATGATATTGAGAAGAT | |
| | | TTATTTGGTTGGGGCAACCGAAGTATAATTAAAGATAACT | |
| | | GCTACTATTTTATACAATGATTTTTGTTTAAATGATAATT | |
| | | GATCCTTAAAAAGATTAAAGACCAAGTTACTTTAGGGATA | |
| | | ACAGCGTAAT | |
| DQ407 | Callinectes | GAGTGACGACAAGACCCTATAAAGCTTTATAAATTTATTT | 404 |
| 678 | marginatus | ATTTCGTCTATTTTCTTTTATAATGATGATGTTAAGAAGG | |
| | | TTTATTTGGTTGGGGCAACCAGAGTATAATTAAAGATAAC | |
| | | TGCTACAATTTAATACAATGATTTTTGTTTATGAAATAAT | |
| | | TGATCCTTGAAAAGATTAAAGATCAAGTTACTTTAGGGAT | |
| | | AACAGCGTAAT | |
| DQ407 | Callinectes | GAGGGACGATAAGACCCTATAAAGCTTTATAAATTTATTA | 405 |
| 677 | affinis | ATTTCGTTTATTTTCTATTATAATAATGATAATAAGAAGA | |
| | | TTTATTTGGTTGGGGCAACCGAAGTATAAGTTTAAATAAC | |
| | | TGCTATAATTTAATACAATGATTTTTGTTTTATAAAATAA | |
| | | TTGATCCTCGAAGAGATTAAAGACCAAGTTACTTTAGGGA | |
| | | TAACAGCGTAAT | |
| DQ407 | Callinectes | GAGGGACGATAAGACCCTATAAAGCTTTATAAATTTATTA | 406 |
| 673 | rathbunae | ATTTCGTTTATTTTTTACTATAATGATGATAATAAGAAGA | |
| | | TTTATTTGGTTGGGGCAACCGAAGTATAAGTTTAAATAAC | |
| | | TGCTATAATTTAATACAATGATTTTTGTTTTAAAAATAAT | |
| | | TGATCCTCGAAGAGATTAAAGACCAAGTTACTTTAGGGAT | |
| | | AACAGCGTAAT | |
| DQ407 | Callinectes | GAGGGACGATAAGACCCTATAAAGCTTTATAAATTTATTA | 407 |
| 674 | bocourti | ATTTCGTTTATTTTCTATTATAATAATGATAATAAGAAGA | |
| | | TTTATTTGGTTGGGGCAACCGAAGTATAAGTTTAAATAAC | |
| | | TGCTATAATTTAATACAATGATTTTTGTTTTAAAAAATAA | |
| | | TTGATCCTCGAAGAGATTAAAGACCAAGTTACTTTAGGGA | |
| | | TAACAGCGTAAT | |
| DQ407 | Callinectes | GAGGGACGATAAGACCCTATAAAGCTTTATAAATTTATTA | 408 |
| 672 | similis | ATTTCTTCTATTTTTTTATAATGATGATGTTAAGAAGATT | |
| | | TATTTGGTTGGGGCAACCGAAGTATAATTAAAGATAACTG | |
| | | CTAATATTTTATACAATGATTTTTGTTTTAATAATAATTG | |
| | | ATCCTTAAAAAGATTAAAGACCAAGTTACTTTAGGGATAA | |
| | | CAGCGTAAT | |
| DQ407 | Callinectes | GAGGGACGATAAGACCCTATAAAGCTTTATAAATTTATTA | 409 |
| 671 | bellicosus | ATTTCGTCTATTTTTTTATAATGATGATGTTGAAAAGATT | |
| | | TATTTGGTTGGGGCAACCGAAGTATAATTTTTAATAACTG | |
| | | CTATAACTTAATACAATGATTTTTGTTTGTTATTATAATT | |
| | | GATCCTTAAAAAGATTAAAGACCAAGTTACTTTAGGGATA | |
| | | ACAGCGTAAT | |
| DQ407 | Callinectes | GAGGGACGATAAGACCCTATAAAGCTTTATAAATTTATTA | 410 |
| 669 | arcuatus | ATTTTGTCTATTTTTCTTATAATGATGATGTTAAGAAGAT | |
| | | TTATTTGGTTGGGGCAACCGAAGTATAATTAAAGATAACT | |
| | | GCTACTATTTTATACAATGATTTTTGTTTTAATAATAATT | |
| | | GATCCTTAAAAAGATTAAAGACCAAGTTACTTTAGGGATA | |
| | | ACAGCGTAAT | |
| EU186 | Metanephrops | GAGGGACGATAAGACCCTATAAAGCTTGATAATTTTATGT | 411 |
| 116 | armatus | ATAAATAAATAAGTTGTTAGTGTTATATTGTTTATCTGTA | |
| | | AAATTATTTCGTTGGGGCGACGATAATATAATTTGTAACT | |
| | | GTTTGGGGGTTAGATTCAAATATGTTTGTGTGTTAATGAT | |
| | | CCATTAATTGTTGATTAAAAATTTAAGTTACTTTAGGGAT | |
| | | AACAGCGTTAT | |
| EU186 | Metanephrops | GAGGGACGATAAGACCCTATAAAGCTTGATAATTTTATAT | 412 |
| 114 | mozambicus | GTAAATAAATAAGTTGTTAGTGTTATATTGTTTACTTACA | |
| | | AAATTATTTCGTTGGGGCGACGATAATATAATTTGTAACT | |
| | | GTTTGAGGTTTAGATCCAAATATATTTGTGTTTAAGTGAT | |
| | | CCATTAATTGTTGATTAAAAATTTAAGTTACTTTAGGGAT | |
| | | AACAGCGTTAT | |
| EU186 | Metanephrops | GAGGGACGATAAGACCCTATAAAGCTTGATAATTTTATAT | 413 |
| 115 | japonicus | ATAAATAAATAAGTTGTTAGTGTTATATTGTTTATCTGTA | |
| | | AAATTATTTCGTTGGGGCGACGATAATATAATTTGTAACT | |
| | | GTTTGGGGGTTAGATTCAAATATGTTTGTGTGTTAATGAT | |
| | | CCATTAATTGTTGATTAGAAATTTAAGTTACTTTAGGGAT | |
| | | AACAGCGTTAT | |
| EU186 | Metanephrops | GAGGGACGATAAGACCCTATAAAGCTTGATAATTTTATAT | 414 |
| 113 | andamanicus | GTAAATAAATAAGTTGTTAGTGTTATATTGTTTATTTATA | |
| | | AAATTATTTCGTTGGGGCGACGATAATATAATTTGTAACT | |
| | | GTTTGAGGTTTAGATCCAAATATATTTGTGTTTAAATGAT | |
| | | CCATTAATTGTTGATTAAAAATTTAAGTTACTTTAGGGAT | |
| | | AACAGCGTTAT | |
| EU186 | Metanephrops | GAGGGACGATAAGACCCTATAAAGCTTGATAATTTTATAT | 415 |
| 112 | velutinus | GTAAATAAATAAGTTGTTAGTGTTATATTGTTTATTTATA | |
| | | AAATTATTTCGTTGGGGCGACGATAATATAATTTGTAACT | |
| | | GTTTGAGGTTTAGATCCAAATATATTTGTGTTTAAATGAT | |
| | | CCATTAATTGTTGATTAAAAATTTAAGTTACTTTAGGGAA | |
| | | AACAGCGTTAT | |
| EU186 | Metanephrops | GAGGGACGATAAGACCCTATAAAGCTTGATAATTTTATAT | 416 |
| 111 | sagamiensis | GTAAATAAATAAGTTGTTAGTGTTATATTGTTTATTTATA | |
| | | AAATTATTTCGTTGGGGCGACGATAATATAATTTGTAACT | |
| | | GTTTGAGGTTTAGATCCAAATATATTTGTGTTTAAATGAT | |
| | | CCATTAATTGTTGATTAAAAATTTAAGTTACTTTAGGGAT | |
| | | AACAGCGTTAT | |
| EU186 | Metanephrops | AAGGGACGATAAGACCCTATAAAGCTTAATAATTTTTGTA | 417 |
| 109 | binghami | AGTAAAATAAATAAATTGTTAGTGATATGCTGCTTACTTA | |
| | | CTAAATTATTTCGTTGGGGTGACGAGGATATAATTTTGTA | |
| | | ACTGTTTTAGTTTAAATTCAGGGATACTTGTACTCAATGA | |
| | | TCCATTTATTTGATGATTAAAAATTTAAGTTACTTTAGGG | |
| | | ATAACAGCGTTAT | |
| EF599 | Parastacus | AAGGGACGATAAGACCCTATAAAGCTTTACATTAAGCGTT | 418 |
| 137 | pugnax | TAAAATTTTAATTAAGTAATAAAATTTTATTAAATGTAAT | |
| | | ATGTTTTGTTGGGGCGACAAGAATATAAAAAATTTAACTG | |
| | | TTCTAGATAATAAGTCAGAAATATCTGATCTTTGAGGACT | |
| | | TTTTATTAAAGTATTAGATTAAGTTACTTTAGGGATAACA | |
| | | GCGTTAT | |
| EF060 | Paranephrops | GAGGGACGATAAGACCCTATAAAGCTTTACATTGAATTTA | 419 |
| 259 | zealandicus | TTAAAAAGTAATTTAAATAATAAAAGTTTATTAGTAAGGA | |
| | | GATGTTTTGTTGGGGTGACAAGAATATAATAAATATAACT | |
| | | GTTCTTTTTTAGTTCAAAGATATTTGAAAAGGAGATCCTT | |
| | | AATAAAGATGTTAGAGTAAGTTACTTTAGGGATAACAGCC | |
| | | GTAA | |
| DQ407 | Callinectes | GAGGGACGATAAGACCCTATAAAGCTTTATAAATTTATTA | 420 |
| 682 | exasperatus | ATTTCGTTTATTTTTTTATAATGATGATGTTAAGAAAGTT | |
| | | TATTTGGTTGGGGCAACCTAAGTATAATTAAAGATAACTG | |
| | | CTACGATTTTATACAATGATTTTTGTTTTAATAGTAATTG | |
| | | ATCCTTGAAAAGATTAAAGACCAAGTTACTTTAGGGATAA | |
| | | CAGCGTAAT | |
| FJ174 | Palinurus | GGGGGACGATAAGACCCTATAAATCTTTATATTTATTATG | 421 |
| 903 | barbarae | ATAACATTAAATATTATAATTGAAAATTTAGTTTTACTGG | |
| | | AATAAAATATTTTGTTGGGGCGACAGGAGTATAAAAAATA | |
| | | ACTGCTTTGTAATATGGGTTAATAATTTTTAATATGTAGT | |
| | | ATAGTTGATCCTTCAGTGAGGATTTTAGAATAAGTTACTT | |
| | | TAGGGATAACAGCGTAAT | |
| NC_02 | Sagmariasus | GGGGGACGATAAGACCCTATAAATCTTTACTCTGGAGACA | 422 |
| 2736 | verreauxi | ATAACATTGAAGATTACTGCTAATAATTCCGTGTTATTGT | |
| | | TTTTAGTGTTTTGTTGGGGCGACAGGAGTATAAATAAATA | |
| | | ACTGCTTTAAACGTATAGTTAATTATAGTTAAGTTGTTGA | |
| | | TAAGTGATCCTTTAGTATGGATTATAGATTAAGTTACTTT | |
| | | AGGGATAACAGCGTAAT | |
| EU882 | Metanephrops | AAGGGACGATAAGACCCTGTAAAGCTTAATAATTCTACGA | 423 |
| 877 | rubellus | GTGGATAAATAAATTGTTTAGTGGCAATGTTACTCACTTG | |
| | | TTAAATTATTTCGTTGGGGTGACGAGGATATAATTTGTAA | |
| | | CTGTCTAAATTTAAAGTCAAGGATGCTTGTATTCGATGAT | |
| | | CCATAATTGGTGATTAAAAATTTAAGTTACTTTAGGGATA | |
| | | ACAGCGTTAT | |
| EU186 | Metanephrops | GAGGGACGATAAGACCCTATAAAGCTTGATAATTTTATAT | 424 |
| 128 | challengeri | ATAATTAAATAAATTATTAGTATTATATTATTTATATATT | |
| | | AAATTATTTCGTTGGGGCGACGATAATATAATTTGTAACT | |
| | | GTTTGAAATTTTAATTCAGAAATATTTGTTTTTAATGATC | |
| | | CTTTTATTATTGATTAAAAATTTAAGTTACTTTAGGGATA | |
| | | ACAGCGTTAT | |
| EU186 | Metanephrops | GAGGGACGATAAGACCCTATAAAGCTTAATAATTTTGTAT | 425 |
| 125 | neptunus | ATAGATAAATAAGTTGTTAGTATTATATTGTCTATTTATT | |
| | | AAATTATTTCGTTGGGGCGACGATAATATAATTTGTAACT | |
| | | GTTTGAAATATTTAATTCAGAAGTATTTGTATTTAATGAT | |
| | | CCTTTTGTTGTTGATTAAAAATTTAAGTTACTTTAGGGAT | |
| | | AACAGCGTTAT | |
| EU186 | Metanephrops | AAGGGACGATAAGACCCTATAAAGCTTGATAATTTTATAT | 426 |
| 124 | australiensis | ATAAGTAAATAAGTTGTTAGTATTATATTGTTTATATATA | |
| | | AAATTATTTCGTTGGGGCGACGATAATATAATTTGTAACT | |
| | | GTTTAATGTTTTGATTCAAATATATTTGTGTTTAATGATC | |
| | | CATTAATTGTTGATTAAAAATTTAAGTTACTTTAGGGATA | |
| | | ACAGCGTTAT | |
| EU186 | Metanephrops | AAGGGACGATAAGACCCTATAAAGCTTGATAATTTTATAT | 427 |
| 123 | arafurensis | ATAAGTAAATAAGTTGTTAGTGTTATATTGTTTATATATA | |
| | | AAATTATTTCGTTGGGGCGACGATAATATAATTTGTAACT | |
| | | GTTTAATGTTTTGATTCAAATATATTTGTGTTTAATGATC | |
| | | CATTAATTGTTGATTAAAAATTTAAGTTACTTTAGGGATA | |
| | | ACAGCGTTAT | |
| EU186 | Metanephrops | GAGGGACGATAAGACCCTATAAAGCTTGATAATTTTATAT | 428 |
| 121 | boschmai | ATAAATAAATAGGTTGTTAGTGTTATATTGTCTATTTATA | |
| | | AAATTATTTCGTTGGGGCGACGATGATATAATTTGTAACT | |
| | | GTTTAAGGTTTTGATTCAAATATATTTGTATTTAATGATC | |
| | | CATTTTTGTTGATTAAAAATTTAAGTTACTTTAGGGATAA | |
| | | CAGCGTTAT | |
| EU186 | Metanephrops | GAGGGACGATAAGACCCTATAAAGCTTGATAATTTTATAT | 429 |
| 117 | formosanus | ATAAATAAATAAGTTGTTAGTGTTATATCGTTTATTTATA | |
| | | AAATTATTTCGTTGGGGCGACGATAATATAATTTGTAACT | |
| | | GTTTAGGTTTAGATTCAAATATATTTGTGTTTTAATGATC | |
| | | CATTAATTGTTGATTAAAAATTTAAGTTACTTTAGGGATA | |
| | | ACAGCGTAAT | |
| EU186 | Metanephrops | GAGGGACGATAAGACCCTATAAAGCTAGATAATTTTGTAT | 430 |
| 118 | sinensis | TTAAATAAATAAGTTGTTAGTGTTATATCATTTATTTATA | |
| | | AAATTATTTCGTTGGGGCGACGATAATATAATTTGTAACT | |
| | | GTTTAGGTTTAGATTCAAATATATTTGTGTTTTATGATCC | |
| | | ATTAATTGTTGATTAAAAATTTAAGTTACTTTAGGGATAA | |
| | | CAGCGTTAT | |
| FJ462 | Lithodes | CAAAAAGACGATAAGACCCTATAAATCTTAACAATAAATA | 431 |
| 647 | ferox | TATTTTATATTTTAGTTTATAAGTGTATAAGGAATAAAAA | |
| | | TATAGGTTTGTTACGCTGGGGGGGCGTAGATATATAAATA | |
| | | AACTGTCTATAGTTTAAATACAATAATCATTGCTTAATAC | |
| | | AAATTGATCCTTAAATAGATTAAAAGATTAAGATACTTTA | |
| | | GGGATAACAGCGTTAT | |
| FJ224 | Oratosquillina | GAGGGACGATAAGACCCTATAAAGCTTTATGTTACATTTC | 432 |
| 280 | interrupta | TTTTTTATAAATTGTATTGTTATTAACTTATTTTAGAATT | |
| | | TGTATCATTTTACTGGGGCGGTAAAAGTATAATAAAGATT | |
| | | AACTGCTTTTGTTTTGAATAACAATTTTAGTTGGAAATAA | |
| | | ATTGATCCATTATTAATGATCATAAGAACAAGTTACTTTA | |
| | | GGGATAACAGCGTAAT | |
| FJ224 | Odontodactylus | GAGGGACGATAAGACCCTATAAAGCTTTATAGTGTAATTA | 433 |
| 282 | japonicus | TTTAATATGAAATATATTAACTTGAAATTATTTAATAAAA | |
| | | AGCACTATTTTACTGGGGCGGTAAAAGTATAACTTAAACT | |
| | | AACTGCTTTTATTTTTAAAAACAATTATAATTGAATATAA | |
| | | ATTGATCCATTATTAATGATCACAAGAACAAGTTACTTTA | |
| | | GGGATAACAGCGTTAT | |
| FJ224 | Miyakella | GAGGGACGATAAGACCCTATAAAGCTTTATGGTATTTTTC | 434 |
| 271 | nepa | TTTTTTATAAATTGTATTGTTATTAACTTATTTTAGAATT | |
| | | TTTATCATTTTACTGGGGCGGTAAAAGTATAATTAGATTA | |
| | | ACTGCTTTTGTTTTAAATAACAATTTTAGTTGGAAATAAA | |
| | | TTGATCCATTATTAATGATCATAAGAACAAGTTACTTTAG | |
| | | GGATAACAGCGTAAT | |
| FJ224 | Erugosquilla | GAGGGACGATAAGACCCTATAAAGCTTTATGATACTTTTC | 435 |
| 263 | woodmasoni | TTTTTTACAAATTATCTCGTTATTAACTTATTTTAGAATT | |
| | | AATATCATTTTACTGGGGCGGTAAAAGTATAATAAAAAGA | |
| | | TTAACTGCTTTTATTTTGTTGAACAATGTTAATTGGAAAT | |
| | | AAACTGATCCATTATTAATGATCATAAGAACAAGTTACTT | |
| | | TAGGGATAACAGCGTAAT | |
| FJ224 | Clorida | AAGGGACGATAAGACCCTATAAAGCTTTATAATATATTTC | 436 |
| 255 | decorata | TTTTACATAAATTGTTTTGTTATTAACGTGTTTTAGATTA | |
| | | TATATTATTTTACTGGGGCGGTAAAAGTATAATAAAAGAT | |
| | | TAACTGCTTTTATTTTGTGTAACAATTGTAGTTGGAAATA | |
| | | AATTGATCCATTATTAATGATTATAAAAACAAGTTACTTT | |
| | | AGGGATAACAGCATAAT | |
| FJ224 | Dictyosquilla | GAGGGACGATAAGACCCTATAAAGCTTTATGGTATTCTTC | 437 |
| 261 | foveolata | TTTTTTATAAATTGTTTTGTTATTAACTTATTTTAGAATT | |
| | | TTTATCATTTTACTGGGGCGGTAAAAGTATAATAAAAGAT | |
| | | TAACTGCTTTTATTTTGTGTAACAATTTTATTTGGAAATA | |
| | | AATTGATCCATTATTAATGATTATAAGAACAAGTTACTTT | |
| | | AGGGATAACAGCGTTAT | |
| FJ224 | Anchisquilla | GAGGGACGATAAGACCCTATAAAGCTTTATAGTATATTTC | 438 |
| 251 | fasciata | TTTTGTATAAATTATTTTGTTACTAACATATTTTAGAACA | |
| | | TATATTATTTTACTGGGGCGGTAAAAGTATAATAAAGACT | |
| | | AACTGCTTTTATTTTGAATAACAATTGTAGTTGGAAATAA | |
| | | ATTGATCCATTATTAATGATTATAAAAACAAGTTACTTTA | |
| | | GGGATAACAGCGTAAT | |
| FJ174 | Scyllarides | GAGGGACGATAAGACCCTATAAATCTTTATATTTTGGGTT | 439 |
| 906 | herklotsii | ATAAGGTTAAAAATTAGGTTTTCATTTAATTTTATAAAGT | |
| | | TAAATATTTTGTTGGGGAGACAGAGGTATAAAGAAAGATA | |
| | | ACTGCCTTAAAATTTATAGTTAAGAATAGTTAAGATAGAA | |
| | | AGTGATCCTTTAATAAGGAAATTAGAATAAGTTACTTTAG | |
| | | GGATAACAGCGTAAT | |
| FJ174 | Palinurus | GGGGGACGATAAGACCCTATAAATCTTTATATTTATTATG | 440 |
| 904 | delagoae | ATAACATTAAATATTATAATTGAAAATTTAGTTTTACTGG | |
| | | AATAAAATATTTTGTTGGGGCGACAGGAGTATAAAAAATA | |
| | | ACTGCTTTGTAATATGGGTTAATAATTTTTAATATGTAGT | |
| | | ATAGTTGATCCTTCAGTGAGGATTTTAGAATAAGTTACTT | |
| | | TAGGGATAACAGCGTAAT | |
| GU727 | Astacus | TAAAGGGACGATAAGACCCTATAAAACTTTATATTTAAAA | 441 |
| 618 | astacus | ATAATTGCTATTATTATTTAAAGAGTTTATTTTTAAATAT | |
| | | TTTATTGGGGTGATAAGGATAAAATATAAGACAACTGTCT | |
| | | TTTATTTTAACAATAATTTTTGAGTAAATGATCTTAATAA | |
| | | AGGAATAAAAGATTAAGTTACTTTAGGGATAACAGCGTAA | |
| | | T | |
| FM208 | Portunus | GGGGGACGATAAGACCCTATAAAGCTTTATAAATCCATAA | 442 |
| 780 | hastatus | ATTATTATATATTTAAGATGATAAAAGTAATCTTTTAGAT | |
| | | ATTTATTTGGTTGGGGCGACAGTGGTATAAATCAATTAAC | |
| | | TGCTAATAAAAATTACAATAATTTTTGATTTCTAAGTTAT | |
| | | AGATCCTCTTTAGGGATTAAAAAATCAAGTTACTTTAGGG | |
| | | ATAACAGCGTTAT | |
| FM208 | Achelous | GGGGGACGATAAGACCCTATAAAGCTTTATAAATCTACAA | 443 |
| 751 | ordwayi | ATTTTATATATTTGTGAATAAAAATAATTTTTCTAATATT | |
| | | TATTTGGTTGGGGCGACAAAAGTGTAATTAAAATAACCGC | |
| | | TAATAATTTGTTACAATGATTTTTGTTTGGCGAAGTTAAT | |
| | | GGATCCCTTTTAAGGATTAAAAAATCAAGTTACTTTAGGG | |
| | | ATAACAGCGTTAT | |
| FM208 | Carcinus | GGGGGACGATAAGACCCTATAAAACTTTATATATGCAACA | 444 |
| 763 | maenas | ATACAGTTGAATTAAAGCATAAAAACTTATTCGATAAAAT | |
| | | ATATTTAGTTGGGGCGACTGAGGTATAATTTATAGTAACT | |
| | | GATTAAAAGCAAGACAAATAATATTTGATTAACTTTAATT | |
| | | GATCCTTTTTAAAGATTAAAAGATTAAGTTACTTTAGGGA | |
| | | TAACAGCGTAAT | |
| FM208 | Portunus | GGGGGACGATAAGACCCTATAAAGCTTTATAAATCTATAA | 445 |
| 752 | inaequalis | ATTATTATATATTTAAAATAATAAAAGTAATTTTTTAGAT | |
| | | ATTTATTTGGTTGGGGCGACAGTGGTATAAATTAATTAAC | |
| | | TGCTAATAAAAATTACAATAATTTTTGATTTTTAAGTCAT | |
| | | AGATCCTCTATAGGGATTAAAAAATCAAGTTACTTTAGGG | |
| | | ATAACAGCGTTAT | |
| FJ965 | Astacoides | AAGGGACGATAAGACCCTATAAAACTTTATATTAGAATTA | 446 |
| 952 | madagas | TTTAAAAACTATTTAGATAGAAAGGTAAGTAAGTAGTAAA | |
| | cariensis | ATATTTTGTTGGGGCGACAAGAATATAAGAATATAACTGT | |
| | | TCTTAAGTATAATCAAAAATTTTTGATTTAATGATCCTAG | |
| | | GTAAAGATATTAGAATAAGTTACTTTAGGGATAACAGCGT | |
| | | AAT | |
| FM207 | Erimacrus | AGGGGACGATAAGACCCTATAAAGCTTTACATACATTATA | 447 |
| 657 | isenbecki | AGTTTGATTTAAGCAAAATTTAAAAATCTAAATTATATAT | |
| | | TATGTTTTGTTGGGGCGACAAAAATATAAGTAATAATAAC | |
| | | TGTTTAATAAAAAAACAATAATAAATGGACAATTAAATGA | |
| | | TCCTTGTTTAAAGATTAAAGACCAAGTTACTTTAGGGATA | |
| | | ACAGCGTTAT | |
| FJ871 | Hemisquilla | GGGGGACGATAAGACCCTATAAAGCTTTATGATAAGGCCT | 448 |
| 141 | australiensis | TTTTGTAAAGAATTTCTATTGTGAAATTACTAAGGGGGTT | |
| | | TATATTATTTTACTGGGGCGGTAAAAGTATATAATTGAAT | |
| | | AACTGCTTTTATTATTGAAAACAATTATAATTGAAATACA | |
| | | AAATGATCCATTATTAATGATTATAAAAACAAGTTACTTT | |
| | | AGGGATAACAGCGTAAT | |
| FJ871 | Austrosquilla | AAGGGACGATAAGACCCTATAAAGCTTTATAGTGTAACTC | 449 |
| 139 | tsangi | TTTTTTACTAGTTTTACTAATATTAAAATATTAAAGAAAT | |
| | | AATACTATTTTACTGGGGCGGTGAGAGTATATAAAAATAA | |
| | | CTGCTTTTATTTTATTAAACAATAATAATTGAAAAATTAA | |
| | | TTGATCCATTTCTAATGATTATAAGAACAAGTTACTTTAG | |
| | | GGATAACAGCGTAAT | |
| FJ462 | Lithodes | CAAAAAGACGATAAGACCCTATAAATCTTTACAATAAATA | 450 |
| 648 | confundens | TATTTTATATTTTAACTTATAAGTGTATAAGAAATAAAAA | |
| | | TATAGATTTGTTTCGCTGGGGCGGCGTAGATATATAAATA | |
| | | AACTGTCTATAATTTAAATACAGTAATTATTGCTTAATAT | |
| | | AAATTGATCCTTAAATAGATTAAAAGATTAAGATACTTTA | |
| | | GGGATAACAGCGTTAT | |
| HM138 | Fallosquilla | GAGGGACGATAAGACCCTATAAAGCTTTATGGTATACCTC | 451 |
| 821 | fallax | TTTTATAAAAATTGTTCTTTATTAACTTATTTTAGATTGT | |
| | | GTATTATTTTACTGGGGCGGTAAAAGTATAATTAAGAGTA | |
| | | ACTGCTTTTATTTTGTATGACAATTATAGTTGAAAATAAA | |
| | | TTGATCCATTATTAATGATTACAAAAACAAGTTACTTTAG | |
| | | GGATAACAGCGTAAT | |
| HM138 | Echinosquilla | GAGGGACGATAAGACCCTATAAAGCTTTATGGTAGTTTCT | 452 |
| 820 | guerinii | TTTTATATAGATTGGTAAAATTTAATTTATTAAAAGAAAA | |
| | | ATATCATTCTACTGGGGCGGTAAAAGTATATCTTTGGTTA | |
| | | ACTGCTTTTGTTTTGTAAGTCAATAATAGTTGAAATATTA | |
| | | ATTGATCCATTATTAATGATCATAAGAACAAGTTACTTTA | |
| | | GGGATAACAGCGTAAT | |
| HM138 | Coronis | GAGGGACGATAAGACCCTATAAAGCTTTATAATACTATCT | 453 |
| 819 | scolopendra | TTTCATATTAATTATCTGGTTATTAACGTACTAAAAGAAA | |
| | | AGTATTATTTTACTGGGGCGGTAAAAGTATATAAAAGAAT | |
| | | AACTGCTTTTGTTTTACAA | |
| | | AACAACTATATTTGAAAAATAAATTGATCCATTATTAATG | |
| | | ATCACAAGAACAAGTTACTTTAGGGATAACAGCGTAAT | |
| HM138 | Chorisquilla | GAGGGACGATAAGACCCTATAAAGCTTTATGATATTTTCT | 454 |
| 818 | tweediei | TTTTATATTAGTTAGTAAAATGTAGTTTATTAAAAGAAAA | |
| | | ATATTATTTTACTGGGGGGGTAGAAGTATATTTTTGATTA | |
| | | ACTGCTTCTGTTTTGTAAGTCAATTATAATTGAAATATGA | |
| | | ATTGATCCATTATTAATGATCATAAGGACAAGTTACTTTA | |
| | | GGGATAACAGCGTAAT | |
| HM138 | Chorisquil | GAGGGACGATAAGACCCTATAAAGCTTTATAATTATTCTT | 455 |
| 817 | la | TTTTATATTAGTTAGTAGAATGTAGTTTATTAAAAAGAAA | |
| | hystrix | TGATTATTTTACTGGGGGGGTAAAAGTATATTTTGATCAA | |
| | | CTGCTTTTGTTTTGTAAATCAATTATAATTGAAATATAAA | |
| | | TTGATCCATTATTAATGATCATAAGAACAAGTTACTTTAG | |
| | | GGATAACAGCGTAAT | |
| HM138 | Chorisquil | GAGGGACGATAAGACCCTATAAAGCTTTATGGTACTTCTT | 456 |
| 816 | laexcavata | TTTTATATTAATTAGGAAAATGTAGTTTATTAAAAAGAAA | |
| | | ATACTATTTTACTGGGGCGGTAAAAGTATATTTTTGATTA | |
| | | ACTGCTTTTGTTTTGTAAGTCAATAATAATTGAAATATTA | |
| | | ATTGATCCATTATTAATGATCACAAGAACAAGTTACTTTA | |
| | | GGGATAACAGCGTAAT | |
| HM138 | Busquilla | GAGGGACGATAAGACCCTATAAAGCTTTATGATATTTCTT | 457 |
| 815 | plantei | TTTCTTATAAATTTTCTTGTTATTAACTTATTTTAGAGCT | |
| | | AATATCATTTTACTGGGGCGGTAAAAGTATAATAAAAGAT | |
| | | TAACTGCTTTTGTTTTGTATAACAATCTTAGTTGGAAATA | |
| | | AATTGATCCATTATTAATGATCATAAGAACAAGTTACTTT | |
| | | AGGGATAACAGCGTAAT | |
| HM138 | Alima | GAGGGACGATAAGACCCTATAAAGCTTTATAATACACGCC | 458 |
| 814 | pacifica | TTCTTTATGAAATATAAAATTATCAAAATATAATAGAACT | |
| | | TGTATTATTTTACTGGGGCGGTAAAAGTATAATAAACTAA | |
| | | CTGCTTTTGTCCTGTATAACAATCTTAATTGAAAATAAAT | |
| | | TGATCCATTATTAATGATCACAAGAACAAGTTACTTTAGG | |
| | | GATAACAGCGTAAT | |
| HM138 | Alima | GAGGGACGATAAGACCCTATAAAGCTTTATAATACAATTC | 459 |
| 813 | orientalis | TTTTTTATAAATTATCGCGTTATTAACCTATCTTAGAATT | |
| | | CGTATTATTTTACTGGGGCGGTAAAAGTATATTAAAGACT | |
| | | AACTGCTTTTATTTTGTGTAACAATTTTAGTTGGAAATAA | |
| | | ATTGATCCATTATTAATGATCACAAGAACAAGTTACTTTA | |
| | | GGGATAACAGCGTAAT | |
| HM138 | Alachosquilla | AAATGACGATAAGACCCTATAAAGCTTTATAATTTTCTCT | 460 |
| 812 | vicina | TTCTATGTAAAGTATAAGAATATTAAAACATTGAAAAGAG | |
| | | TAAATTATTTTACTGGGGCGGTAAAAGTATAAAAAATAAC | |
| | | TGCTTTTATTTTGAATAACAACTATTATTGGAATTTGAAT | |
| | | TGATCCATTAATAATGATCACAAGAACAAGTTACTTTAGG | |
| | | GATAACAGCGTAAT | |
| HM138 | Gonodact | GAGTGACGATAAGACCCTATAAAGCTTTATGGTATACCTT | 461 |
| 822 | ylellusespinosus | TTTTATACAAATTACATATTATTAACCTATTAAAAAAGTA | |
| | | ATATCATTTTACTGGGGCGGTAAAAGTATATTCGCTTAAC | |
| | | TGCTTTTATTATTTAAAACAATAATAATTGAACAATCCAT | |
| | | TGATCCATTAATAATGATCATAAGGCCAAGTTACTTTAGG | |
| | | GATAACAGCGTAAT | |
| HM138 | Gonodactylellus | GAGGGACGATAAGACCCTATAAAGCTTTATAATACATTTC | 462 |
| 823 | affinis | TTTTACATAAATTATTAATTATTAACTTGTTAAAGAAATA | |
| | | ATATTATTTTACTGGGGGGGTAAAAGTATATTTTATTTGA | |
| | | ACAACTGCTTTTGTTTTAAAAAACAATTATAATTGAACAA | |
| | | CTCATTGATCCATTAATAATGATCATAAGGCCAAGTTACT | |
| | | TTAGGGATAACAGCGTAAT | |
| HM138 | Kempella | GAGGGACGATAAGACCCTATAAAGCTTTATAATATTTTTC | 463 |
| 833 | mikado | TTTCTTATAAATTATATTGTTATTAACATATTTTAGAATT | |
| | | TGTATTATTTTACTGGGGGGGTAAAAGTATAATAAAATAA | |
| | | CTGCTTTTATTTTGTATAACAATCTTAATTGGTAATAAAT | |
| | | TGATCCATTATTAATGATTATAAGAACAAGTTACTTTAGG | |
| | | GATAACAGCGTAAT | |
| HM138 | Hemisquilla | GGGGGACGATAAGACCCTATAAAGCTTTATGATAAGGCCT | 464 |
| 832 | californiensis | TTTTGTAAAGAATTTCTATTGTGAAATTACTAGGGGGTTT | |
| | | ATATCATTTTACTGGGGGGGTAAAAGTATATAATTGAATA | |
| | | ACTGCTTTTATTATTGAAAACAATTATAATTGAAATACAA | |
| | | AATGATCCATTATTAATGATTATAAAAACAAGTTACTTTA | |
| | | GGGATAACAGCGTAAT | |
| HM138 | Haptosquilla | GAGGGACGATAAGACCCTATAAAGCTTTATGATAGTTTCT | 465 |
| 831 | trispinosa | TTTTATAATAATTAGTAAAATGTAATTTATTAAAAGAAAT | |
| | | ATATTATTTTACTGGGGCGGTAAAAGTATATTTTTGATTA | |
| | | ACTGCTTTTGTTTTGCAAAACAATAATAATTGAATTATAA | |
| | | ATTGATCCATTATTAATGATCATAAGAACAAGTTACTTTA | |
| | | GGGATAACAGCGTAAT | |
| HM138 | Haptosquilla | GAGTGACGATAAGACCCTATAAAGCTTTATGGTAGTTTCT | 466 |
| 830 | glyptocercus | TTTTATAGTAATTAGTATAATGTAATTTATTAAAGGGAAA | |
| | | TATATCATTTTACTGGGGCGGTAAAAGTATATTTTTGATT | |
| | | AACTGCTTTTGTTTTGTAAAACAATGATAATTGAATTATA | |
| | | AATTGATCCATTATTAATGATTACAAGAACAAGTTACTTT | |
| | | AGGGATAACAGCGTAAT | |
| HM138 | Gonodactylus | GAGGGACGATAAGACCCTATAAAGCTTTATGTTATATATT | 467 |
| 828 | platysoma | CTTTTATGTAATCTATAATTATTAACTCATTGAAGAAATA | |
| | | ATATCATTTTACTGGGGCGGTAAAAGTATATTTGAATAAC | |
| | | TGCTTTTGTTATAAAAAACAATAATAATTGAGTAATCCAT | |
| | | TGATCCATTATTAATGATCATAAGACCAAGTTACTTTAGG | |
| | | GATAACAGCGTAAT | |
| HM138 | Gonodactylaceus | GAGGGACGATAAGACCCTATAAAGCTTTATAGTAATACCT | 468 |
| 827 | falcatus | TTTTATTTAAAATATAAAATGTTAACTTGTTAAAAGAGTA | |
| | | TTATTATTTTACTGGGGGGGTAAAAGTATATAATGATTAA | |
| | | CTGCTTTTGTCTTACTAAACAATAATAATTGAATAATTAT | |
| | | GTGATCCATTATTAATGATCAAAAGTACAAGTTACTTTAG | |
| | | GGATAACAGCGTAAT | |
| HM138 | Gonodactylus | GAGGGACGATAAGACCCTATAAAGCTTTATAATATAATTC | 469 |
| 825 | childi | TTTGATATGAAGTGTTACTTATTAATTTATTAAAGAAATA | |
| | | CTATTATTTTACTGGGGCGGTAAAAGTATATATTTGGAGT | |
| | | AACTGCTTTTGTTATTTTAAACAATAATAATTGAGTGTCA | |
| | | TATTGATCCACTAATAGTGAATAAAAGAACAAGTTACTTT | |
| | | AGGGATAACAGCGTAAT | |
| HM138 | Gonodactylellus | GAGGGACGATAAGACCCTATAAAGCTTTATAATACGTTTC | 470 |
| 824 | annularis | TCTTACACAAATTATAAATTGTTAACTTGTTAAAGAATAA | |
| | | GTATTATTTTACTGGGGCGGTAAAAGTATACCATAGACTA | |
| | | ACTGCTTTTGTTATTAAAAACAATAATAATTGGATAATCG | |
| | | ATTGATCCATTAATAATGATCATAAGGCCAAGTTACTTTA | |
| | | GGGATAACAGCGTAAT | |
| HM138 | Odontoda | GAGGGACGATAAGACCCTATAAAGCTTTATGATACAACTA | 471 |
| 842 | ctylusscyllarus | TTTCTTATAAAATATCTTTTAAATCACTTAATAGAATGTA | |
| | | TTATTTTACTGGGGCGGTAAAAGTATAATGTATTATTAAC | |
| | | TGCTTTTGTTTTTAAAAACAATCATAATTGGAAACAAATT | |
| | | GATCCATTATTAATGATCACAAGAACAAGTTACTTTAGGG | |
| | | ATAACAGCGTTAT | |
| HM138 | Odontodactylus | AAGGGACGATAAGACCCTATAAAGCTTTATAATAAAATCA | 472 |
| 841 | latirostris | TCTTCTATGGAATAAGTGTTATTAAATTAGCTGATGAGGT | |
| | | ATATTATTTTACTGGGGCGGTAAAAGTATAAACGATTAAC | |
| | | TGCTTTTGTTCTTAAAAACAATTATAATTGAAAACATATT | |
| | | GATCCATTATTAATGATCACAAGAACAAGTTACTTTAGGG | |
| | | ATAACAGCGTTAT | |
| HM138 | Odontodactylus | AAGGGACGATAAGACCCTATAAAGCTTTATGATAAAGTTA | 473 |
| 840 | havanensis | TCCTCTACGGAATAAACATTATTAAACTAGCTGATAACGC | |
| | | ATATCATTTTACTGGGGCGGTAAAAGTATAATAAGATTAA | |
| | | CTGCTTTTGTTTTTACAAACAATTATAATTGAAAACAGAT | |
| | | TGATCCATTATTAATGATCACAAGAACAAGTTACTTTAGG | |
| | | GATAACAGCGTTAT | |
| HM138 | Odontodactylus | GAGGGACGATAAGACCCTATAAAGCTTTATATTACATATA | 474 |
| 839 | cultrifer | TTTTATATAAAATGTTTATTATTAAATTATTTAATGAAAT | |
| | | GTATTATTTTACTGGGGCGGTAAAAGTATATTTGAGTAAC | |
| | | TGCTTTTGCTTTTAAAAACAATCATAATTGAAAGTAAATT | |
| | | GATCCATTATTAATGATCACAAGAACAAGTTACTTTAGGG | |
| | | ATAACAGCGTTAT | |
| HM138 | Neogonodactylus | GAGGGACGATAAGACCCTATAAAGCTTTATGGTAATATTT | 475 |
| 838 | oerstedii | TTGTATATAAAATATTTCATGTAACTTATTAAAAAATAAA | |
| | | CACCATTTTACTGGGGCGGTAAAAGTATATTTTTGATTAA | |
| | | CTGCTTTTGTTGTTGAAAACAGTTGTAGCTGAATAATAAA | |
| | | TTGATCCATTATTAATGATCACAAGTACAAGTTACTTTAG | |
| | | GGATAACAGCGTAAT | |
| HM138 | Neogonodactylus | GAGGGACGATAAGACCCTATAAAGCTTTATGGTAAAATTT | 476 |
| 837 | bredini | TTATATATAAAATATTTCGTGTAACTTATTAAAAAGTAAA | |
| | | TACCATTTTACTGGGGGGGTAAAAGTATATCTTGATTAAC | |
| | | TGCTTTTATTATTAAAAACAGTTATAGCTGAATAATAAAT | |
| | | TGATCCATTACTAATGTTCATAAGTTCAAGTTACTTTAGG | |
| | | GATAACAGCGTAAT | |
| HM138 | Neogonodactylus | GAGGGACGATAAGACCCTATAAAGCTTTATGGTAAAATTT | 477 |
| 836 | bahiahondensis | TTGTATATAAATTATATAATATTAAATTATTAAAAAATAA | |
| | | ATACCATTTTACTGGGGCGGTAAAAGTATATTTTTGATTA | |
| | | ACTGCTTTTGTTATTAAAAACAGTCGTAGCTGAATAATCA | |
| | | GTTGATCCATTATTAATGATCATAAGTTCAAGTTACTTTA | |
| | | GGGATAACAGCGTAAT | |
| HM138 | Lysiosquil | AAGGGACGATAAGACCCTATAAAGCTTTATGATTACAATC | 478 |
| 835 | linasulcata | TTTTAGTGTTGATTATTTTAATATTAAAGTATTAGAAAAT | |
| | | TATATCATTTTACTGGGGCGGTAAAAGTATAAAGGAATAA | |
| | | CTGCTTTTGTTGAGATTAAACAACGGTAGTTGGAAAATAA | |
| | | ATTGATCCATTAATAATGATCACAAGAACAAGTTACTTTA | |
| | | GGGATAACAGCGTAAT | |
| HM138 | Squilla | GAGGGACGATAAGACCCTATAAAGCTTTATAATATTTCTT | 479 |
| 854 | rugosa | TTTTATATAAATTGTGTTATTGTTAACTTATTTTAAAGAT | |
| | | TTTATTATTTTACTGGGGGGGTAAAAGTATATTAAAGATT | |
| | | AACTGCTTTTGTTTTTTATAACAATCGTAATTGGAAATAA | |
| | | ATTGATCCATTATTAATGATCACAAGAACAAGTTACTTTA | |
| | | GGGATAACAGCGTAAT | |
| HM138 | Raoulserenea | GAGTGACGATAAGACCCTATAAAGCTTTATAGTACTGTTT | 480 |
| 852 | hieroglyphica | TTCTTTATAAATTAATTTAGTATTAACATGTCTAAAAATG | |
| | | TCTACTATTTTACTGGGGCGGTAAGAGTATAATAGGTTAA | |
| | | CTGCTTTTTGTATGTACAACAATGATACTTGAAATATAAT | |
| | | TGATCCACGATTAGTGATTATAAAACCAAGTTACTTTAGG | |
| | | GATAACAGCGTAAT | |
| HM138 | Raoulserenea | GAGTGACGATAAGACCCTATAAAGCTTTATAGTATGAGTT | 481 |
| 851 | oxyrhyncha | TTTTTTATAAATTAACGTGATATTAACGTACTTAAGTTTT | |
| | | CTTACTATTTTACTGGGGCGGTAAAAGTATAATGGATTAA | |
| | | CTGCTTTTTCTGTGTAAAACAAGGATGTTTGAAATATAAT | |
| | | TGATCCACAATTAGTGATTATAAAACCAAGTTACTTTAGG | |
| | | GATAACAGCGTAAT | |
| HM138 | Pseudosquillopsis | GAGGGACGATAAGACCCTATAAAGCTTTATAGTGTTTTTT | 482 |
| 845 | marmorata | TTTTATATAAATTGTGCTAATTTAGTTTATAAAAAAAAAA | |
| | | CATACTATTTTACTGGGGCGGTAAAAGTATTTATTATTGA | |
| | | AAAACTGCTTTTGTTTTTAAAAACAAATATGATTGGAAAT | |
| | | AAATTGATCCATTATTAATGATTATAAAAACAAGTTACTT | |
| | | TAGGGATAACAGCGTAAT | |
| HM138 | Raoulserenea | GAGTGACGATAAGACCCTATAAAGCTTTATAGTACTGTTT | 483 |
| 850 | ornata | TTCTTTATAAATTAATTTAGTATTAACATGTCTAAAAATG | |
| | | TCTACTATTTTACTGGGGCGGTAAGAGTATAATAGATTAA | |
| | | CTGCTTTTTGTATGTACAACAATGATACTTGAAATATAAT | |
| | | TGATCCACGATTAGTGATTATAAAACCAAGTTACTTTAGG | |
| | | GATAACAGCGTAAT | |
| HM138 | Raoulserenea | GAGTGACGATAAGACCCTATAAAGCTTTATAGTATGAGTT | 484 |
| 849 | komaii | TTTTTTATAAATTAACGTGATATTAACGTACTTAAGTTTT | |
| | | CTTACTATTTTACTGGGGCGGTAAAAGTATAATGGATTAA | |
| | | CTGCTTTTTCTGTGTAAAACAAGGATGTTTGAAATATAAT | |
| | | TGATCCACAATTAGTGATTATAAAACTAAGTTACTTTAGG | |
| | | GATAACAGCGTAAT | |
| HM138 | Protosquilla | AAGGGACGATAAGACCCTATAAAGCTTTATAGTGGCCTCT | 485 |
| 843 | folini | TTTTATATAAATTGGTAAAATATAGTTTATTAAAAGAAGA | |
| | | ATACTATTTTACTGGGGCGGTAAAAGTATATTTTTGATTA | |
| | | ACTGCTTTTGTTTTGAATGTCAATAATAGTTGAGTAATAA | |
| | | GTTGATCCATTATTAATGATCACAAGAACAAGTTACTTTA | |
| | | GGGATAACAGCGTAAT | |
| NC_04 | Ibacus | AGGGGACGATAAGACCCTATAAATCTTTATATTTTAAATT | 486 |
| 1153 | alticrenatus | TGTTAGTTTAAAAAATGGTTTAAATTTTGATTTAATAAAT | |
| | | TAAAATATTTTGTTGGGGAGACAGGAGTATAAAAAGTAAC | |
| | | TGCTTTATAATTTAAGTTAATAATATTTAGTTAAGTGATC | |
| | | CTTTAATATGGATTTTAGATTAAGTTACTTTAGGGATAAC | |
| | | AGCATAAT | |
| JN701 | Scyllarides | GAGGGACGATAAGACCCTATAAATCTTTATATTTTGCCTT | 487 |
| 692 | nodifer | ATAAGGTTAAAAATTAGGTTCTTATTTAATTTTATAAGGT | |
| | | TAAATATTTTGTTGGGGAGACAGAGGCATAAAAAAAGTAA | |
| | | CTGCCTTAAAGTTTAAGTTAAAAATGTTTAAGTTGTTAAG | |
| | | TGATCCTTTAATAAGGAAATTAGAATAAGTTACTTTAGGG | |
| | | ATAACAGCGTAAT | |
| MN817 | Scyllarides | GAGGGACGATAAGACCCTATAAATCTTTATATTTTGGCTT | 488 |
| 127 | haanii | ATAAGGTTAAAAATTAGGTTATAATTTAGTTTTATAAGGT | |
| | | TGAATATTTTGTTGGGGAGACAGGAGTATAAAAAGAAATA | |
| | | ACTGCTTTAGAATTAGAGTTAAAGGTGTTTAAGTTTAAAA | |
| | | GTGATCCTTTAATAAGGAATGTAGATTAAGTTACTTTAGG | |
| | | GATAACAGCGTAAT | |
| JN701 | Scyllarides | GAGGGACGATAAGACCCTATAAATCTTTATATTTTGGTTT | 489 |
| 689 | brasiliensis | ATACGGTTAAAAGTTAGGTTATTATTTAATTTTATAAGTT | |
| | | TGAATATTTTGTTGGGGAGACAGGGGCATAAAAGGAAATA | |
| | | ACTGCCTTAAAGTTTAAGTTAAAAATGTTTAAATTATTAA | |
| | | GTGATCCTTTAATAAAGAATATAGATTAAGTTACTTTAGG | |
| | | GATAACAGCGTAAT | |
| NC_05 | Takuspinosoc | GAGGGACGATAAGACCCTATAAAGCTTTATAGTGTAGTTT | 490 |
| 0686 | arinatus | TTTTATATTAATTATATATTTTAGTTTATTAAAAAAGTAA | |
| | | TACTATTTTACTGGGGCGGTAAAAGTATATTTTTGATTAA | |
| | | CTGCTTTTGTTTTTAAAAACAATAATAATTGGTTAATAGA | |
| | | TTGATCCATTATTAATGATCACAAGAACAAGTTACTTTAG | |
| | | GGATAACAGCGTAAT | |
| JN566 | Jasus | GGGGGACGATAAGACCCTATAAATCTTTATACTAGTTAGT | 491 |
| 222 | frontalis | AATAACATTAAAGATTAATTTTATATTTTTTTGTTAGTAG | |
| | | TTTTAGTATTTTGCTGGGGCGGCAGGGGTATAATTCAATA | |
| | | ACTGCTCTATAGTTGTAGTTAATTATGTTTAAATTGTTGG | |
| | | TAGTTGATCCTTTAGTGGAGATTATAGATTAAGTTACTTT | |
| | | AGGGATAACAGCGCAAT | |
| HM637 | Menippe | GAGGGACGATAAGACCCTATAAAGCTTTATATGTTATTTA | 492 |
| 974 | mercenaria | AATTTAATTGAATTGATTTATAAATTTTAAATTTAGAGTA | |
| | | ATTTATTTTGCTGGGGCGGCATAAGTATAAGTTATATTAA | |
| | | CTGCTTAATAATGATACAATAATAGTTGAATTGTTGTTAA | |
| | | ATGATCCTTTTTAAAGATTTAAGAACAAGTTACTTTAGGG | |
| | | ATAACAGCGTTAT | |
| JF737 | Procambarus | AAGGGACGATAAGACCCTATAAAACTTTATATTTATAATA | 493 |
| 171 | paeninsul | TAGTAGTTAGTTTTATTTAATAGTACTATTTTAGAGTATT | |
| | anus | TGGTTGGGGTGACAAGGATAAAATATAAAATAACTGTCTT | |
| | | TTTTTTTTACAGTAATGTTTGTTTTAATGATCCTAAAAGG | |
| | | GATTAAAGATTAAGTTACTTTAGGGATAACAGCGTAAT | |
| JQ229 | Puerulus | GGGGGACGATAAGACCCTATAAATCTTTATATTTGCCGTA | 494 |
| 876 | sewelli | GAAAAGAGTGTGTATTAGGGCTAAAGATATATTATTTTGT | |
| | | TGGGGCGATATTTTGTTGGGGCGACAAGGGTATAATGAGT | |
| | | AACTGCTTTAAAAGTTTAATTAGATAATATCTAGTTTGAG | |
| | | AATAAGAGATCCTTTACTAAGGATTGTTAAAATAAGTTAC | |
| | | TTTAGGGATAACAGCGTAAT | |
| JQ229 | Panulirus | GGGGGACGATAAGACCCTATAAATCTTTATGTTTTATCTA | 495 |
| 873 | polyphagus | GTGAATTTCAGAGATTAGTTACATATTTGGTGTTACTACG | |
| | | GTAAAATATTTTGTTGGGGCGACAGGAGTATAACAAGTAA | |
| | | CTGCTTTGGATATAGAATTAATTATTTTTATAGTGAGAAG | |
| | | TTAAATTGATCCTCTTCTGGAGAAATCAGACTAAGTTACT | |
| | | TTAGGGATAACAGCGTAAT | |
| MN817 | Panulirus | GGGGGACGATAAGACCCTATAAATCTTGATGGTCAAAATA | 496 |
| 128 | longipes | TTAATTTTTATGTAGTAGTTATAAGCTTAAATTTAATACT | |
| | | TTGACTATTTTGTTGGGGCGACAGGGGTATAATTAGTAAC | |
| | | TGCTTTGAGTAAAGAATTAATTATTTTTATTGTGTTGTGT | |
| | | TTGAGATCCTTCTTTGAAGATATCAGATCAAGTTACTTTA | |
| | | GGGATAACAGCGTAAT | |
| MT533 | Panulirus | GGGGGACGATAAGACCCTATAAATCTTTATGTTTTAACTA | 497 |
| 488 | penicillatus | ATAAATTTTAGGGATTAGTTATATACCTAGTATTATTAGT | |
| | | TGAAATATTTTGTTGGGGCGACAGGAGTATAATAAGTAAC | |
| | | TGCTTTGTTTATAAAATTAATTGTTTTTATTGTGGAAATT | |
| | | GGTATGATCCTCTTTAGAGAAATCAGACTAAGTTACTTTA | |
| | | GGGATAACAGCGTAAT | |
| JN701 | Panulirus | GGGGGACGATAAGACCCTATAAATCTTTATAGCTTTGATT | 498 |
| 738 | interruptus | ATAAGTTTTAGAGATTAGTTATAAGTCTAAAATTATTTTT | |
| | | TGAGCTATTTTGTTGGGGCGACAAAAGTATAACAAGTAAC | |
| | | TGCTTTGGTTAATAAATTAATTATTTTTATAATGTTATTT | |
| | | TAAAGATCCTTCTTTGAAGATGACAGAATAAGTTACTTTA | |
| | | GGGATAACAGCGTAAT | |
| JN701 | Panulirus | GGGGGACGATAAGACCCTATAAATCTTGATAATTAAAGTA | 499 |
| 739 | marginatus | ATAGTTTTTACGTAGTAGTTATAAGCTTAAATCTAATGCT | |
| | | TTGATTATTTTGTTGGGGCGACAAGGGTATAATAAATAAC | |
| | | TGCCTTGAATAGATAGCTAATTATTTTTATTATGTTATGT | |
| | | TTAAGATCCTTCTTTGAAGATATCAGATCAAGTTACTTTA | |
| | | GGGATAACAGCGTAAT | |
| JN701 | Ibacus | AGGGGACGATAAGACCCTATAAATCTTTATATTTAAAGTT | 500 |
| 700 | peronii | TGTTAATTTAAAAAGTAGATTAAAATTTTAATTTAATAAA | |
| | | TTTAAATATTTTGTTGGGGAGACAGGAGTATAAAAAATAA | |
| | | CTGCTTTAAAGTTTGAGTTAATAATATTTAATTAAGTGAT | |
| | | CCTTTACTATGGATTTTAGATTAAGTTACTTTAGGGATAA | |
| | | CAGCATAAT | |
| JN701 | Ibacus | AGGGGACGATAAGACCCTATAAATCTTTATATTTGGAGTT | 501 |
| 698 | chacei | TGTTAATTTGAAAAGTAAAATTAAAATTTTAATTTAATGA | |
| | | ATTTAAATATTTTGTTGGGGAGACAGGAGTATAAAAAATA | |
| | | ACTGCTTTAAAATTTGAGTTAATAATATTTAATTAAGTGA | |
| | | TCCTTTATTATGGATTTTAGATTAAGTTACTTTAGGGATA | |
| | | ACAGCATAAT | |
| KF220 | Charybdis | GAGGGACGATAAGACCCTATAAAGCTTTATAAATTTGAAG | 502 |
| 508 | hellerii | ATTTTGTGTATTTTTATATAAAAATGAATTTTGAAGATTT | |
| | | TATTTGGTTGGGGCGACAATGGTATAATGAGAATAACTGC | |
| | | TATTAAAATTAACAATTATATTTGAATTATTAATAATTGA | |
| | | TCCTTTATAAGGATTTTAAGATCAAGTTACTTTAGGGATA | |
| | | ACAGCGTTAT | |
| KC237 | Faxonella | AAGGGACGATAAGACCCTATAAAACTTTATATTTAATAAT | 503 |
| 200 | clypeata | AGTAATTAATTTTATTTTAAGAGTATTATTTGAAAGTATT | |
| | | TGGTTGGGGTGACAAGGATAAAATATAAAATAACTGTCTT | |
| | | TTTTTTATTACAATAATATTTGGTTTAATGATCCTAAAAT | |
| | | GGATTAAAGATTAAGTTACTTTAGGGATAACAGCGTAAT | |
| KC163 | Fallicambarus | AAGGGACGATAAGACCCTATAAAACTTTATATTTTATAAG | 504 |
| 440 | kountzeae | TGGTAGTTAATTTTATTTTGTAATACTATTTAGGAGTATT | |
| | | TGGTTGGGGTGACAAAGATAAAATAAAAAATAACTGTCTT | |
| | | TTTTATTTACAGTAATGTTTGATTTAATGATCCTTAAAAG | |
| | | GATTAAAGATTAAGTTACTTTAGGGATAACAGCGTAAT | |
| JX123 | Arenaeus | GAGGGACGATAAGACCCTATAAAGCTTTATAAGTGTAGGT | 505 |
| 471 | mexicanus | ATTTTGTATATTTTATGTTATAATAATAATTTGTTGAATA | |
| | | CTTATTTGGTTGGGGCAACAAGAGTATAAAGAGAGTAACT | |
| | | GCTAAGATTTAAAACAATTATATTTGATTAAAAAGTAATT | |
| | | GATCCCAAGAGGATTAAAAGATCAAGCTACTTTAGGGATA | |
| | | ACAGCGTAAT | |
| JX514 | Cambarus | AAGGGACGATAAGACCCTATAAAACTTTATATTTAGGAGT | 506 |
| 540 | tartarus | AGAAGTTAGTTTTATTTTATTGGCTCTATTTAAAAGTATT | |
| | | TGGTTGGGGTGACATAAATAAAAAATGAAATAATTGTTTT | |
| | | TATTTTTTACAATGATATTTGGGTTTGTGATCCTGAAGTG | |
| | | GATTGAAAGATTAAGTTACTTTAGGGATAACAGCGTAAT | |
| JQ407 | Chionoecetes | GGGGGACGATAAGACCCTATAAAGCTTTACATATAAGTAG | 507 |
| 454 | tanneri | AGTTTACTTAATTAAAAAGTAAAAGTTTAATCTAATTAAT | |
| | | GTGTTTTGTTGGGGCGACATAAATATAATTTATATTAACT | |
| | | GTTTATAAATTAATACAATAATAAATGATTCTAATGTAAT | |
| | | TGATCCTTATTAAAGATTAAAAGACTAAGTTACTTTAGGG | |
| | | ATAACAGCGTTAT | |
| JQ229 | Thenus | AGGGGACGATAAGACCCTATAAATCTTTATATTAATTTAT | 508 |
| 907 | unimaculatus | TGTTAAGCTGGAAAATAGAATTAATTCGTGGGGTAACAAT | |
| | | TTTAAATATTTTGTTGGGGAGACAGAAGTATAAAAGAATA | |
| | | ACTACTTTGGATAAAGAGTTAATGATATTTAATGTAGAGA | |
| | | CCCTTTCCTAAGGGTTATAGAACAAGTTACTTTAGGGATA | |
| | | ACAGCGTAAT | |
| JQ229 | Thenus | GGGGGACGATAAGACCCTATAAATCTTTATATTAATTTGT | 509 |
| 878 | indicus | TGTTAAATTGGAAAATAGAATTAATTTCGGGCTTAACAAT | |
| | | TTTAAATATTTTGTTGGGGAGACAGAAGTATAAATAAATA | |
| | | ACTACTTTAAAGAGAAAGTTAATGATATTTAATGTAGAGA | |
| | | TCCTTTACTAAGGATTGTAGAACAAGTTACTTTAGGGATA | |
| | | ACAGCGTAAT | |
| KM074 | Haptosquilla | GAGTGACGATAAGACCCTATAAAGCTTTATAGTAGTGTCT | 510 |
| 036 | hamifera | TTTTATAATAATTAGTAAAATGTAATTTATTGAAAGAAGT | |
| | | ATACTATTTTACTGGGGCGGTAAAAGTATATTTTTTGATT | |
| | | AACTGCTTTTGTTTTGCAAGACAATGATAATTGAATTATG | |
| | | AATTGATCCATTATTAATGATTATAAGAACAAGTTACTTT | |
| | | AGGGATAACAGCGTAAT | |
| KJ132 | Lithodes | CAAAAAGACGATAAGACCCTATAAATCGTTACGCTGGGGC | 511 |
| 573 | turritus | GGCGTAGATATATAAATAAACTGTCTATAATTTAGATACA | |
| | | ATAATCATTGAATTGATCCTTAAATAGATTAAAAGATTAA | |
| | | GATACTTTAGGGATAACAGCGTTAT | |
| KF828 | Bouchardina | AAGGGACGATAAGACCCTATAAAACTTTATATTTAAGAGT | 512 |
| 212 | robisoni | AGTAGTTAGTTTTGTTTAAGGAGTAGTATTTAAAAATATT | |
| | | TGGTTGGGGTGACAAGGATAGAATAAAAAATAACTGTCTT | |
| | | TTTTTTTTACAGTAATATTTGGTTTAATGATCCTAAAAAG | |
| | | GATTAAAGATTAAGTTACTTTAGGGATAACAGCGTAAT | |
| KF828 | Troglocambarus | AAGGGACGATAAGACCCTATAAAACTTTATATTTATAAGG | 513 |
| 201 | maclanei | TAGTAAGTTAGTTTTATTTAAAGGTACTATGTTAGAATGT | |
| | | TTGGTTGGGGTGACAAGGATAAAATATAAAATAACTGTCT | |
| | | TTTTTTTTTACAGTAATGTTTGGTTTAATGATTCTAAAAG | |
| | | GGATTAAAAGATTAAGTTACTTTAGGGATAACAGCGTAAT | |
| KF828 | Hobbseus | AAGGGACGATAAGACCCTATAAAACTTTATATTTGAAAAT | 514 |
| 192 | yalobushensis | AGTAGTTAATTTTGTTTAGTAAAGTAGTATTTAAAAGTAT | |
| | | TTGGTTGGGGTGACAAGGATAGAATATAAAATAACTGTCT | |
| | | TTTTTTTTTACAATAATATTTGTTTTGATGATCCTGAAAT | |
| | | GGATTAAAGATTAAGTTACTTTAGGGATAACAGCGTAAT | |
| KF828 | Hobbseus | AAGGGACGATAAGACCCTATAAAACTTTATATTTGAAAAT | 515 |
| 190 | prominens | AGTAGTTAATTTTATTTAATAAAGTATTATTTAAAAGTAT | |
| | | TTGGTTGGGGTGACAAGGATAGAATATAAAATAACTGTCT | |
| | | TTTTTTTTTTACAATAATATTTGTTTTAATGATCCTGAAA | |
| | | TGGATTAAAGATTAAGTTACTTTAGGGATAACAGCGTAAT | |
| KF220 | Charybdis | GAGGGACGATAAGACCCTATAAAGCTTTATAAATTTGAAG | 516 |
| 510 | lucifera | ATTTTGTGTATTTTTATATAAAAATGAATTTTGAAGATTT | |
| | | TATTTGGTTGGGGCGACAATGGTATAATGAGAATAACTGC | |
| | | TATTAAAATTAACAATTATATTTGAATTATTAATAATTGA | |
| | | TCCTTTATAAGGATTTTAAGATCAAGTTACTTTAGGGATA | |
| | | ACAGCGTTAT | |
| KF828 | Hobbseus | AAGGGACGATAAGACCCTATAAAACTTTATATTTGAAAAT | 517 |
| 189 | petilus | AGTAGTTAATTTTATTTAATAAAGTATTATTTAAAAGTAT | |
| | | TTGGTTGGGGTGACAAGGATAGAATATAAAATAACTGTCT | |
| | | TTTTTTTTTACAATAATATTTGTTTTAATGATCCTGAAAT | |
| | | GGATTAAAGATTAAGTTACTTTAGGGATAACAGCGTAAT | |
| KX237 | Faxonella | AAGGGACGATAAGACCCTATAAAACTTTATATTTAATAAT | 518 |
| 946 | creaseri | AGTAATTAATTTTATTTTAAGAAGTATTATTTGAAAGTAT | |
| | | TTGGTTGGGGTGACAAGGATAAAATATAAAATAACTGTCT | |
| | | TTTTTTATTACAATAATATTTGGTTTAATGATCCTAAAAT | |
| | | GGATTAAAGATTAAGTTACTTTAGGGATAACAGCGTAAT | |
| KU130 | Thranita | AAGGGACGATAAGACCCTATAAAGCTTTATAAGTTTAGAG | 519 |
| 124 | danae | ATTTTATATATTTTTCTATAGCAATAATTTCTTGAAAGCT | |
| | | TATTTGGTTGGGGCGACAATAGTATAATTGAAATAACTGC | |
| | | TTTAAAATAATACAAATATATTTGAATTATAATTAGTTGA | |
| | | TCCTTTATAAAGATTTAAGATCAAGTTACTTTAGGGATAA | |
| | | CAGCGTTAT | |
| KT365 | Monomia | AGGGGACGATAAGACCCTATAAAGCTTTATAAATTTAAAG | 520 |
| 606 | petrea | ATTTTAGAGATTTTATACCTAAATTTATTTTTTGATAGTT | |
| | | TATTTGGTTGGGGCGACAAAGGTATAATTATGGATAACTG | |
| | | CCATTAATGTTTATACATATATCTTTGATTTAAGGCTTAA | |
| | | ATAATAATTGATCCTTTTTAAGGATTTAAAGATCAAGTTA | |
| | | CTTTAGGGATAACAGCGTAAT | |
| KT001 | Neogonodactylus | GAGGGACGATAAGACCCTATAAAGCTTTATGGTAGAATTT | 521 |
| 544 | wennerae | TTATATATAAATTATAAAATATCAACTTATTAGAAAATAA | |
| | | ATACCATTATACTGGGGCGGTAAAAGTATATTTTTGATTA | |
| | | ACTGCTTTTGTTGTTAAAAACAGTTGTGGCTGAATAATCA | |
| | | GTTGATCCATTATTAATGATCATAAGTTCAAGTTACTTTA | |
| | | GGGATAACAGCGTAAT | |
| KR026 | Xiphonectes | AGGGGACGATAAGACCCTATAAAACTTTATAAGTTTAAAG | 522 |
| 905 | pseudohastatoides | ATCTCTACTATTTTTTTTTATAAAGTTTTTTTTGATAGCT | |
| | | TATTTGGTTGGGGCGACAAAGGTATAATTATGAATAACTG | |
| | | CCATTAAAATTAATACATTTATTGATGATTGTTGATAAAT | |
| | | AGTTGATCCTTTTTAAAGATTAAAAGATCAAGTTACTTTA | |
| | | GGGATAACAGCGTAAT | |
| KT001 | Gonodactylellus | GAGGGACGATAAGACCCTATAAAGCTTTATAATATATTTC | 523 |
| 545 | viridis | TTTTATTTAAATTATAGTTTTTAACTCATTAAAGAAAATA | |
| | | TATTATTTTACTGGGGCGGTAAAAGTATATTTTTGATTAA | |
| | | CTGCTTTTGTTCTAACAAACAATTATAATTGAATATCCAT | |
| | | TGATCCATTATTAATGAGCATAAGACCAAGTTACTTTAGG | |
| | | GATAACAGCGTAAT | |
| KT001 | Gonodactylaceus | AAGTGACGATAAGACCCTATAAAGCTTTATAGTAGTTCCT | 524 |
| 543 | ternatensis | TTCTATTTAGATTATAAAATTTCAACTTTTTTAAAGGCTA | |
| | | TTACTATTTTACTGGGGCGGTAAAAGTATATTATGATTAA | |
| | | CTGCTTTTGTCTTGACAAACAATAGTAATTGGATAATTAT | |
| | | GTGATCCATTATTAATGATCAAAAGTACAAGTTACTTTAG | |
| | | GGATAACAGCGTAAT | |
| KR153 | Belosquilla | ATGACGAACAAACCCATACTATAGCTTTATAATATGTTCT | 525 |
| 534 | laevis | TCTTATGTAAGTTATCCCGCTTATTAAACTATTTTAAGAA | |
| | | TCATATTATTTTACCGGGGGGTAATGGTATAATTTGTCGA | |
| | | GATTAACTGCTTTTCTTTTGTATGACATCTTTACTTGACA | |
| | | ACTAATTGATCCATTATTAATGATCACAAGAACAAGTTAC | |
| | | TTTAGGGATAACAGCGTACT | |
| KX238 | Procambarus | AAGGGACGATAAGACCCTATAAAACTTTATATTTATAATA | 526 |
| 053 | okaloosae | TAGTAGTTAGTTTTATTTAATAGTACTATTTTAGAGTATT | |
| | | TGGTTGGGGTGACAAGGATAAAATATAAAATAACTGTCTT | |
| | | TTTTTTTTTACAATAATGTCTGTTTTAATGATCCTAAAAG | |
| | | GGATTAAAGATTAAGTTACTTTAGGGATAACAGCGTAAT | |
| KX238 | Procambarus | AAGGGACGATAAGACCCTATAAAACTTTATATTTATAAGA | 527 |
| 050 | morrisi | TATTAGTTAGTTTTATTTAAAAGTATTATATTAAAATATT | |
| | | TGGTTGGGGTGACAAGGATAGAATACAAAATAACTGTCTT | |
| | | TTTTTTTTACAGTAATGTTTGGTTTAATGATTCTAAAGGG | |
| | | ATTAAAAGATTAAGTTACTTTAGGGATAACAGCGTAAT | |
| KX238 | Procambarus | AAGGGACGATAAGACCCTATAAAACTTTATATTTATAATA | 528 |
| 049 | milleri | TAGTAATTAGTTTTATTTAATAATATTATATTAAAGTATT | |
| | | TGGTTGGGGTGACAAGGATATAATATAAAATAACTGTCTT | |
| | | TTTTTTTTACAGTAATATTTGGTTTAATGATCCTAGAAGG | |
| | | GATTAAAAGATTAAGTTACTTTAGGGATAACAGCGTAAT | |
| KX238 | Procambarus | AAGGGACGATAAGACCCTATAAAACTTTATATTTATAAAA | 529 |
| 048 | mancus | TAGTAGTTAGTTTTGTTTAGAGGTATTAATTTAGAGTATT | |
| | | TGGTTGGGGTGACAAGGATAAAATATAAAATAACTGTCTT | |
| | | TTTTTATTACAGTAATGTTTGGTTTAATGATCCTAAAAGG | |
| | | GATTTAAGATTAAGTTACTTTAGGGATAACAGCGTAAT | |
| KX238 | Procambarus | AAGGGACGATAAGACCCTATAAAACTTTATATTTATAAGT | 530 |
| 046 | lunzi | AGTAGTTAGTTTTATTTAAAAATATTATATTAAAATATTT | |
| | | GGTTGGGGTGACAAGGATAAAATATAAAATAACTGTTCTT | |
| | | TTTTTTTTTACAGTAATGTTTGATTTAATGATCCTAAAAA | |
| | | GGATTAAAAGATTAAGTTACTTTAGGGATAACAGCGTAAT | |
| KX237 | Hobbseus | AAGGGACGATAAGACCCTATAAAACTTTATATTTGAGAAT | 531 |
| 948 | cristatus | AGTAGTTAATTTTGTTTAGTAAAGTATTATTTAAAAGTAT | |
| | | TTGGTTGGGGTGACAAGGATAGAATATAAAATAACTGTCT | |
| | | TTTTTTTTTACAATAATATTTGTTTTAATGATCCTGGAAT | |
| | | GGATTAAAGATTAAGTTACTTTAGGGATAACAGCGTAAT | |
| KX237 | Procambarus | AAGGGACGATAAGACCCTATAAAACTTTATATTTATAAGG | 532 |
| 996 | acutissimus | TAGTAGTTAGTTTTATTTTAAAGTATTATTTTAGAGTATT | |
| | | TGGTTGGGGTGACAAGGATAAAATATAAAATAACTGTCTT | |
| | | TTTTTTTTTACAGTAATGTTTGGTTTAATGATCCTAAAAG | |
| | | GGATTAAAGATTAAGTTACTTTAGGGATAACAGCGTAAT | |
| KX237 | Faxonius | AAGGGACGATAAGACCCTATAAAACTTTATATTTAGAAGT | 533 |
| 973 | pagei | AGAAGTTAATTTTATTTTAGTATTACTATTTAAAATATTT | |
| | | GGTTGGGGCGACGAGGATAAAAGGTTAAATAACTGTCTTT | |
| | | TTTTTTTTACAATAATATTTGGTTTAATGAGCCTAAAAGG | |
| | | GATTTAAGATTAAGTTACTTTAGGGATAACAGCGTAAT | |
| KY236 | Manningia | GAGGGACGATAAGACCCTATAAAGCTTTATAATAATTTTT | 534 |
| 044 | pilaensis | TTTTATAAAAATTATGGTATTATAACTTATTAAAAAATCA | |
| | | TTATTATTTTACTGGGGCGGTAAAAGTATAATTTAGATTA | |
| | | ACTGCTTTTAACATAAAAACCAATAATAATTGAGAACAAT | |
| | | TTGATCCATTATTAATGATCATAAGAACAAGTTACTTTAG | |
| | | GGATAACAGCGTAAT | |
| KX279 | Pontastacus | AGGGGACGATAAGACCCTATAAAACTTTATATTTTAAAAT | 535 |
| 350 | leptodactylus | AATAATTAATTTTTATATAGAAGATTTATTTTTAAATATT | |
| | | TTATTGGGGTGATAAAGATAAAATGTGAGATAACTGTCTT | |
| | | TTTTTTTTTACAATTAATATTTGAGTAAAAGATCTTAACA | |
| | | AAGGAAATAAGAGATTAAGTTACTTTAGGGATAACAGCGT | |
| | | AAT | |
| KX238 | Procambarus | AAGGGACGATAAGACCCTATAAAACTTTATATTTATAAGA | 536 |
| 091 | zonangulus | TAGTAGCTAGTTTTATTTAAAAGTATTATTTTGGAGTATT | |
| | | TGGTTGGGGTGACAAGGATAAAATATAAAATAACTGTCTT | |
| | | TTTTTTTTTACAGTAATGTTTGGTTTAATGATCCTAAAAG | |
| | | GGATTAAAGATTAAGTTACTTTAGGGATAACAGCGTAAT | |
| KX238 | Procambarus | AGGGACGATAAGACCCTATAAAACTTTATATTTATAATAT | 537 |
| 089 | youngi | GGTAATTAATTTTATTTAAAAATATTATTTTAGAGTATTT | |
| | | GGTTGGGGTGACAAGGATAAAATGTTAAATAACTGTCTTT | |
| | | TTTTTTTTACAGTAATGTTTGGTTTAATGATCCTACAAAG | |
| | | GATTAAAGATTAAGTTACTTTAGGGATAACAGCGTAAT | |
| KX238 | Procambarus | AAGGGACGATAAGACCCTATAAAACTTTATATTTATAAGG | 538 |
| 075 | seminolae | TAGTAGATAGTTTTATTTATAAGTATTATATTAGAATATT | |
| | | TGGTTGGGGTGACAAGGATAAAATATAAAATAACTGTCTT | |
| | | TTTTTTTTACAGTAATATTTGGTTTAATGATCCTAAAATG | |
| | | GATTAAAAGATTAAGTTACTTTAGGGATAACAGCGTAAT | |
| KX238 | Procambarus | AGGGACGATAAGACCCTATAAAACTTTATATTTCTAACGT | 539 |
| 063 | pycnogonopodus | AGTAATTAGTTTTATTTGAGAGTACTATATTAAAGTATTT | |
| | | GGTTGGGGTGACAAGGATAAAATATAAAATAACTGTCTTT | |
| | | TTTTTTTACAGTAATATTTGGTTTAATGATCCTTAAAAGG | |
| | | ATTAGAAGATTAAGTTACTTTAGGGATAACAGCGTAAT | |
| KX238 | Procambarus | GAGGGACGATAAGACCCTATAAAACTTTATATTTATAAGG | 540 |
| 054 | orcinus | TAGTAATTAGTTTTATTTAAAAGTATTATATTAGAATATT | |
| | | TGGTTGGGGTGACAAGGATAAAATATAGAATAACTGTCTT | |
| | | TTTTTTTTACAGTAATATTTGGTTTAATGGGCCTGAAAAG | |
| | | GATTAAAAGATTAAGTTACTTTAGGGATAACAGCGTAAT | |
| KX238 | Procambarus | AGGGACGATAAGACCCTATAAAACTTTATATTTATAAGGT | 541 |
| 055 | pallidus | AGTAATTAGTTTTATTTAAAAGTATTATATTAGAATATTT | |
| | | GGTTGGGGTGACAAGGATAAAATACAGAATAACTGTCTTT | |
| | | TTTTTTTACAGTAATATTTGGTTTAATGGGCCTGAAAAGG | |
| | | ATTAAAAGATTAAGTTACTTTAGGGATAACAGCGTAAT | |
| MH168 | Alima | GGGGGACGATAAGACCCTATAAAGCTTTATATTACGTTTC | 542 |
| 208 | maxima | TTCTTTATAAAATATCCGGTTGTTAACTTATTTTAGAAAT | |
| | | TGTATTATTTTACTGGGGCGGTAAAAGTATAAATAAGATT | |
| | | AACTGCTTTTGTCTTGCATAACAATTTTAATTGGAAATAA | |
| | | ATTGATCCATTAATAATGATCACAAGAACAAGTTACTTTA | |
| | | GGGATAACAGCGTAAT | |
| MF490 | Scyllarides | GAGGGAYGATAAGACCCTGTAAATCTTTATATTTTGGCTG | 543 |
| 148 | deceptor | ATAAGGTTAAAAGTTAGGTTAKTATTTAATTTTATAAGTT | |
| | | TGAATATTTTGTTGGGGAGACAGGGGYATAAAAGGAAATA | |
| | | ACTGCCTTARAGTTTAAGTTAAAAATGTTTAAATTATTAA | |
| | | GTGATCCTTTAATAAGGAATATAGACTAAGTTACTTTAGG | |
| | | GATAACAGCGTAAT | |
| KY524 | Monomia | AGGGGACGATAAGACCCTATAAAGCTTTATAAGTTTAGGA | 544 |
| 476 | argentata | ATTTTAAATATTTTGTAGATAAATTTATTTTCCAATATCT | |
| | | TATTTGGTTGGGGCGACAAAGGTATAATTATGAATAACTG | |
| | | CTATTATTAACAATACATCTATCGTTGGCTAAAATTTATA | |
| | | ATAATTGATCCTCTTTAGAGATTAAAAGATCAAGTTACTT | |
| | | TAGGGATAACAGCATAAT | |
| KY524 | Xiphonectes | GAGGGACGATAAGACCCTATAAAGCTTTATATATTAAATG | 545 |
| 461 | pulchricristatus | ATTTTATGGATTTAAAAAATAAAAATTATTCTGAAAAATT | |
| | | TATTTGGTTGGGGCGACAGAGGTATAATTTAGATAACTGC | |
| | | TAGGAATTTATACAATTATTCTTGAATTATGTTTGTAAAT | |
| | | GATCCTTGTTAGAGATTAAAAGATCAAGTTACTTTAGGGA | |
| | | TAACAGCGTAAT | |
| NC_04 | Paralithodes | AAAAGACGATAAGACCCTATAAATCTTTACAATAAATATA | 546 |
| 2240 | platypus | TTTTATATTTTAGCTTATAAGTAAATAAGAAATAAAAATA | |
| | | TAGGTTTGTTACGCTGGGGGGGCGTAGATATATAAATAAA | |
| | | CTGTCTATAGTTTAAATACAATAATTATTGCTTAATACAA | |
| | | ATTGATCCTTAAATAGATTAAAAGATTAAGATACTTTAGG | |
| | | GATAACAGCGTTAT | |
| KY426 | Lopholithodes | AAGACGATAAGACCCTATAAATCTTTACAATAAATATATT | 547 |
| 330 | foraminatus | TTATATTTTAGCTTATAAGTGTATAAGAAATAAAAATATA | |
| | | GATTTGTTGCGCTGGGGGGGCGTAGATATATAAATAAACT | |
| | | GTCTATAATTTAAATACAATAATCATTGTTTAATACAAAT | |
| | | TGATCCTTAAATAGATTAAAAGATTAAGATACTTTAGGGA | |
| | | TAACAGCGTTAT | |
| KY236 | Faughnia | GAGGGACGATAAGACCCTATAAAGCTTTATGATTCCTTTT | 548 |
| 047 | formosae | TTTATTATAAATTATATATTTTAGTTTACTAAAAAACAGG | |
| | | TATTATTTTACTGGGGCGGTAAAAGTATGTTTTTGGGTAA | |
| | | CTGCTTTTATTTTTAAAAACAATTATGTTTGGAAATAAAA | |
| | | TGGTCCATTATTAATGATTATAAGAACAAGTTACTTTAGG | |
| | | GATAACAGCGTAAT | |
| KY236 | Faughnia | GGGGGACGATAAGACCCTATAAAGCTTTATGGTAGTTTCT | 549 |
| 046 | profunda | TTTAATATGAATTGTATGATTTCTAGTTTATTGAAGAAAA | |
| | | ATTACTATTTTACTGGGGCGGTAAAAGTATGCTTTCGGTT | |
| | | AACTGCTTTTGTTTTTAAAAACAAACATGGTTGGAAATAA | |
| | | ATTGATCCATTATTAATGATTATAAGAACAAGTTACTTTA | |
| | | GGGATAACAGCGTAAT | |
| KY236 | Bathysquilla | GAGGGACGATAAGACCCTATAAAGCTTTATGGTGTACTAC | 550 |
| 045 | crassispinosa | TTTGTTACAAAATATAATAATTAAAACTTATTGAAGTAGT | |
| | | GGGCATTATTTTACTGGGGCGGTAAAAGTATAAATAATCT | |
| | | GAGTAACTGCTTTTATTGTGCTAAACAATTATAATTGACT | |
| | | TTTAAATTGATCCATTACTAATGATCACAAGAACAAGTTA | |
| | | CTTTAGGGATAACAGCGTAAT | |
| NC_00 | Eriocheirsinensis | TAAAGGGACGATAAGACCCTATAAAGCTTAATATTAAATT | 551 |
| 6992 | | TTATATAGTCAAATTTTAGGTTATAAAGATTTTGTAAATT | |
| | | GAATTTATTTTATTGGGGCGATAAGAGTAAAATGATTATT | |
| | | AACTGCTTAACATTAAGTACACTTATGTGTGATTAAAATT | |
| | | TTAAATGATCCTAATTAAAGATTAAAAGTTTAAGTTACTT | |
| | | TAGGGATAACAGCGTTAT | |
| NC_00 | Harpiosquilla | GAGGGACGATAAGACCCTATAAAGCTTTATGACATATTTT | 552 |
| 6916 | harpax | TTTTTATAAATTATTTTGTTATTAACTTAATATAAAATGT | |
| | | ATGTCATTTTACTGGGGCGGTAAAAGTATAATTAAAGAAT | |
| | | AACTGCTTTTGTTTTTAATAACAATTATAGTTGGAAATGA | |
| | | ATTGATCCATTATTAATGATCACAAGAACAAGTTACTTTA | |
| | | GGGATAACAGCGTAAT | |
| NC_00 | Callinectes | CCATAGGGACGATAAGACCCTATAAAGCTTTATAAATTTA | 553 |
| 6281 | sapidus | TTAATTTCGTTTATTTTTTATTATAATAATGATGATAAGA | |
| | | GAATTTATTTGGTTGGGGCAACCGAAGTATAATTTTAAAT | |
| | | AACTGCTATAATTTAATACAATGATTTTTGTCTTATACAA | |
| | | TAATTGATCCTCAAAGAGATTAAAGACCAAGTTACTTTAG | |
| | | GGATAACAGCGTAAT | |
| NC_00 | Squilla | GAGGGACGATAAGACCCTATAAAGCTTTATAATACTTCTT | 554 |
| 6081 | mantis | TTTTATATAAATTGTTCTATTATTAACTTATTTTAAAGAT | |
| | | TGTATTATTTTACTGGGGGGGTAAAAGTATATTAAAGACT | |
| | | AACTGCTTTTATTTTTTATAACAATTATGATTGAATGTAA | |
| | | ATTGATCCATTATTAATGATCACAAGAACAAGTTACTTTA | |
| | | GGGATAACAGCGTAAT | |
| NC_00 | Portunus | GAGGGACGATAAGACCCTATAAAGCTTTATAAATTTAGAA | 555 |
| 5037 | trituberculatus | ATTTTATATATTTTTTTAATAATGATAATCTTTTGAAAAT | |
| | | TTATTGGGTTGGGGCAACAAAAGTATAATTATTAATAACT | |
| | | GCTAGAAAGTATTACAATTGTTTTTGTCATAAAGAAAATT | |
| | | AGTTGATCCCTGAAAGGGATTAAAAGATCAAGTTACTTTA | |
| | | GGGATAACAGCGTAAT | |
| NC_00 | Panulirus | GGGGGACGATAAGACCCTATAAATCTTGATGATTAAGATA | 556 |
| 4251 | japonicus | ATAAATTTTAGGCAGTAGTAATAAGTTTAAATTTAATGTC | |
| | | TTAATTATTTTGTTGGGGCGACAGGAGTATAAGAAGTAAC | |
| | | TGCTTTGAAAAAATAATTAATTATTTTTATGATGTTTTGT | |
| | | TTGTGATCCTTCTATGAAGATAATAGATCAAGTTACTTTA | |
| | | GGGATAACAGCGTAAT | |
| MN334 | Cancer | AGGGGACGATAAGACCCTATAAAGCTTTATATTTTAGCTA | 557 |
| 534 | pagurus | TAATTTAATTAAATTAATTAGATCAAAGGTTTTAATAAGT | |
| | | TATTATATTTTATTGGGGAGGTATAAGTATAATTTATGTA | |
| | | ACTGCTTATAAGTTAGTACACTGATAATTGATTAGAGTTT | |
| | | CAATGATCCTTTAAGATAAAGATTTAAGACTAAGTTACTT | |
| | | TAGGGATAACAGCGTTAT | |
| MT750 | Chionoecetes | GGGGGACGATAAGACCCTATAAAGCTTTACATATAAGTAG | 558 |
| 295 | japonicus | AGTTTACTTAATTAAAAAGTAAAAGTTTAATCTAATTAAT | |
| | | GTGTTTTGTTGGGGCGACATAAATATAATTTATATTAACT | |
| | | GTTTATAATTTAATACAATAATAAATGATTTTAATGTAGT | |
| | | TGATCCTTATTAAAGATTAAAAGACTAAGTTACTTTAGGG | |
| | | ATAACAGCGTTAT | |
| NC_01 | Scylla | GAGGGACGATAAGACCCTATAAAGCTTTATAAATTAAGTA | 559 |
| 2567 | tranquebarica | AGTTAGAACAAATTTTATAGAATAAAGGTAAATTTAATAT | |
| | | ATTTATTTGGTTGGGGCGACAATGGTATAAATTAAATAAC | |
| | | TGCTATTAAAATTAAACAATTATATTTGATTAAAATTATA | |
| | | ATTGATCCTTATTATGGATTAAAAGATCAAGTTACTTTAG | |
| | | GGATAACAGCGTAAT | |
| NC_01 | Scylla | CTAGAGGGACGATAAGACCCTATAAAGCTTCATAAGTTAA | 560 |
| 2565 | serrata | ATAAATTAGATGGATTTTTAGAATAAAAGTAAATTTAGTA | |
| | | AGCTTATTTAGTTGGGGCGACAAGGGTATAAAATTAAATA | |
| | | ACTGCTATTAAGGTTAGACAATTATATTTGGTTGGATTGT | |
| | | AATTGATCCTCGCTATGGATTAAAAGATTAAGTTACTTTA | |
| | | GGGATAACAGCGTAAT | |
| NC_01 | Eriocheir | AAGGGACGATAAGACCCTATAAAGCTTAATATTAAATTTT | 561 |
| 1598 | hepuensis | ATATAGTCAAATTTTAAATTATAAAGATTTTGTAAATTGA | |
| | | ATTTATTTTATTGGGGCGATAAGAGTAAAATGATTATTAA | |
| | | CTGCTTAACATTAAGTACACTTATATGTGATTAAAATTTT | |
| | | AAATGATCCTAATTAAAGATTAAAAGTTTAAGTTACTTTA | |
| | | GGGATAACAGCGTTAT | |
| NC_01 | Eriocheir | AAGGGACGATAAGACCCTATAAAGCTTAATATTAAATTTT | 562 |
| 1597 | japonica | ATTTAGTCAAATTTTAGGTTATAAAGATTTTGTAAATTGA | |
| | | ATTTATTTTATTGGGGCGATAAGAGTAAAATGATTATTAA | |
| | | CTGCTTAATATTAAGTACACTTATATGTGATTAAAATTTT | |
| | | AAATGATCCTAATTAAAGATTAAAAGTTTAAGTTACTTTA | |
| | | GGGATAACAGCGTTAT | |
| NC_01 | Cherax | GGGGGACGATAAGACCCTATAAAGTTTGACATTAAATTAA | 563 |
| 1243 | destructor | TTAAGGGTAATTTAGATTATAAAGTCTTATTATTATATAA | |
| | | GTGTTTAGTTGGGGCGACTAGGATATAAGTTATTTAACTG | |
| | | TTTCTTCATTCGAATCAAAAATTTTTGATTTTATGATCCT | |
| | | TTTTAAGGGTACTAGAGTAAATTACTTTAGGGATAACAGC | |
| | | GTAAT | |
| >NC_0 | Squilla | GAGGGACGATAAGACCCTATAAAGCTTTATGATATTTCTT | 564 |
| 07444 | empusa | TTTTGTATAAAATATTTATTGTTAACATATTTAAAAGTTT | |
| | | ATATTATTTTACTGGGGCGGTAAAAGTATATTAAAGACTA | |
| | | ACTGCTTTTGTTTTTTATAACAATTATAATTGAAAATAAA | |
| | | TTGATCCATTATTAATGATCATAAGAACAAGTTACTTTAG | |
| | | GGATAACAGCGTAAT | |
| NC_00 | Lysiosquillina | AAGGGACGATAAGACCCTATAAAGCTTTATGATGTATATT | 565 |
| 7443 | maculata | CGCTAATGTTGATTTTCGTAATATTAAAGCGTTAGAGAAT | |
| | | CATATCATTTTACTGGGGGGGTAAAAGTATAAAAGAATAA | |
| | | CTGCTTTTGTTGAAATTAAACAATGATAGTTGGAGAATAA | |
| | | ATTGATCCATTAATAATGATCATAAGAACAAGTTACTTTA | |
| | | GGGATAACAGCGTAAT | |
| NC_00 | Gonodactylus | GAGGGACGATAAGACCCTATAAAGCTTTATAATGGATTCT | 566 |
| 7442 | chiragra | TGTTATATAAATTATCAATTGTTAACATGTTAAAAGAATA | |
| | | ATATTATTTTACTGGGGGGGTAAAAGTATATATCTGAGTA | |
| | | ACTGCTTTTGTTTTTTAAAACAGTGATGACTGACTTATTA | |
| | | ATTGATCCACTAATAGTGAGCATAAGGACAAGTTACTTTA | |
| | | GGGATAACAGCGTAAT | |
| NC_01 | Panulirus | GGGGGACGATAAGACCCTATAAATCTTTATGTTTTAACTA | 567 |
| 6015 | homarus | ATAAATTTTAGAGATTAGTTAAATATTTAGTGTTATTAAG | |
| | | TTAAATATTTTGTTGGGGCGACAGGAGTACAATAAGTAAC | |
| | | TGCTTTGTTTATAAAGTTAATTATTTTTATTATGGAAATT | |
| | | GTTATTGATCCTTTTTGGAGAAATCAGAATAAGTTACTTT | |
| | | AGGGATAACAGCGTAAT | |
| NC_01 | Homarus | TAAAGGGACGATAAGACCCTATAAAGCTTAATAATTTAGT | 568 |
| 5607 | americanus | ATATAATTAGATGAGTTGAAAGTTTAATATTATTTATATA | |
| | | CTAAATTATTTCGTTGGGGCGACGATGATATAATTTGTAA | |
| | | CTGTTTAAATTTAAAATACAGAGATATTTGTGTGTAATGA | |
| | | TCCTTGTTGTTGATTAAAAATTTAAGTTACTTTAGGGATA | |
| | | ACAGCGTTAT | |
| NC_01 | Panulirus | GGGGGACGATAAGACCCTATAAATCTTTATACTTTAACTA | 569 |
| 4854 | ornatus | ATAAATTTTAAAGATTAGTTGCAAATTTAGTGTTATTAAG | |
| | | TTAAAATATTTTGTTGGGGCGACAGGAGTATAATAAGTAA | |
| | | CTGCTTTGTTTATAAATTTAATTATTTTTAATGTGTAAAT | |
| | | TGTATTGATCCTCTGTTGGAGAAATCAGACTAAGTTACTT | |
| | | TAGGGATAACAGCGTAAT | |
| NC_01 | Oratosquilla | GAGGGACGATAAGACCCTATAAAGCTTTATGATACTATTC | 570 |
| 4342 | oratoria | TTTTTTATAAATTGTTTTGTTATTAACTTATTTTAGAATT | |
| | | AGTATTATTTTACTGGGGCGGTAAAAGTATAATAAAAGAA | |
| | | TAACTGCTTTTGTTTTGTATAACAATTTTAGTTGGAAATA | |
| | | AATTGATCCATTATTAATGATCATAAGAACAAGTTACTTT | |
| | | AGGGATAACAGCGTAAT | |
| NC_01 | Panulirus | GGGGGACGATAAGACCCTATAAATCTTTATATTTTATTTA | 571 |
| 4339 | stimpsoni | ATAAATTTTAAAAATTAGTTATACATTTAGCGATATTAAG | |
| | | TCAAATATTTTGTTGGGGCGACAGGAGTATAATAAGTAAC | |
| | | TGCTTTGTTTGTAAAGTTAATTATTTTTATTGTGTAGAGT | |
| | | TAAATTGATCCTTTCTAGAGAAATCAGATCAAGTTACTTT | |
| | | AGGGATAACAGCGTAAT | |
| NC_01 | Charybdis | GAGGGACGATAAGACCCTATAAAGCTTTATAAATTTAAAG | 572 |
| 3246 | japonica | ATTTTATATATTTTTATATAAAAATAAATTCCAGAGGATT | |
| | | TATTTTGTTGGGGCGACAATGGTATAATGAAAATAACTGC | |
| | | TATTAAAATTAACAATTATATTTGTTTATGTAAATAATTG | |
| | | ATCCTTTATAAGGATTTTAAGATCAAGTTACTTTAGGGAT | |
| | | AACAGCGTTAT | |
| NC_01 | Scylla | GAGGGACGATAAGACCCTATAAAGCTTTATAAGTTAAGTA | 573 |
| 2572 | paramamosain | AGTTAAATTAATTTTTTAAAATAAAAATAAGCTTAAAAAA | |
| | | TTTATTTGGTTGGGGCGACAATGGTATAAATTAAATAACT | |
| | | GCTATTAAAATTAAGCAATTATATTTGATTAGAATTATAA | |
| | | TTGATCCTTATTATGGATTAAAAGATCAAGTTACTTTAGG | |
| | | GATAACAGCGTAAT | |
| NC_01 | Scylla | GAGGGACGATAAGACCCTATAAAGCTTTATAGATTGAGTG | 574 |
| 2569 | olivacea | GATTAAACCAATTTTTTAGAATAAAAGTAAATTTGGAAGA | |
| | | TTTATTTGGTTGGGGCGACAAATGTATAATGTTAAATAAC | |
| | | TGCTATTAAATTTAAACAATTATATTTGATAAAAATTTAA | |
| | | ATGATCCTTAATAAAGATTAAAAGATCAAGTTACTTTAGG | |
| | | GATAACAGCGTAAT | |
| NC_02 | Cherax | GAGGGACGATAAGACCCTATAAAGTTTATACATGAAGTTG | 575 |
| 2937 | quadricarinatus | GTTAAGAGTGATTTAAGGTGTTAAAGTTTATTATCAGCAG | |
| | | GGTGTTTAGTTGGGGCGACTAGGATATAAATTATATAACT | |
| | | GTTTTTTGTTTAAATCAGAGATATTTGTTCATATGATCCC | |
| | | TTTTTAGGATTAGAGAATAAATTACTTTAGGGATAACAGC | |
| | | GTAAT | |
| NC_02 | Cherax | AAGGGACGATAAGACCCTATAAAGTTTATACATCCAAATG | 576 |
| 2936 | cainii | ATTGAAAATAATTAAGAAAATTAAAGTTTATTATTAGGGA | |
| | | GATGTTTAGTTGGGGCGACTAGGATATAAGTTAATTTAAC | |
| | | TGTCCTAAATTAAAATCAAAGATATTTGAGTTTGTGATCC | |
| | | TTTTTTAGGATTAAAGAACAAGTTACTTTAGGGATAACAG | |
| | | CGTAAT | |
| NC_02 | Paralithodes | CAAAAAGACGATAAGACCCTATAAATCTTTACAATAAATA | 577 |
| 1458 | brevipes | TATTTTATATTTTATATTATAAGTGTATAATAAATAAAAA | |
| | | TATAAATTTGTTGCGCTGGGGCGGCGTAGATATATAAATA | |
| | | AACTGTCTATAATTTAAATACAATAATCATTGTTTAGTAA | |
| | | AATTGATCCTTAAATAGATTAAAAGATTAAGATACTTTAG | |
| | | GGATAACAGCGTTAT | |
| NC_02 | Paralithodes | CAAAAAGACGATAAGACCCTATAAATCTTTACAATAAATA | 578 |
| 0029 | camtschaticus | TATTTTATATTTTAGTTTATAAGTGAATGAGATATAAAAA | |
| | | TATAGGTTTGTTGCGCTGGGGCGGCGTAGATATATAAATA | |
| | | AACTGTCTATAGTTTAAATACAATAATCATTGCTTAATAT | |
| | | AAATTGATCCTTAAATAGATTAAAAGATTAAGATACTTTA | |
| | | GGGATAACAGCGTTAT | |
| NC_02 | Scyllarides | GAGGGACGATAAGACCCTATAAATCTTTATATTTTGGGTT | 579 |
| 0022 | latus | ATAAGGTTAAAAATTAGGTTATCTTTTAATTTTATAAGGT | |
| | | TAAATATTTTGTTGGGGAGACAGAGGTATAAAAAGAGATA | |
| | | ACTGCCTTAAAATTTATAGTTAAGAATAGTTAAGATTGAA | |
| | | AGTGATCCTTTAATAAGGAAAATAGAAGAAGTTACTTTAG | |
| | | GGATAACAGCGTAAT | |
| NC_01 | Procambarus | GGAGGGACGATAAGACCCTATAAAACTTTATATTTATAAT | 580 |
| 6926 | clarkii | ATAGTAGTTAGTTTTATTTAAGGGTATTATTTTAGAGTAT | |
| | | TTGGTTGGGGTGACAAGGATAAAATATTAAATAACTGTCT | |
| | | TTTTTTTTTACAGTGATGTTTGGTTTAATGATCCTAAAAG | |
| | | GGATTAAAGATTAAGTTACTTTAGGGATAACAGCGTAAT | |
| NC_02 | Procambarus | AAGGGACGATAAGACCCTATAAAACTTTATATTTATAAAG | 581 |
| 0021 | fallax | TAGTAGTTAGTTTTATTTAAAAGTATTATATTAGAATATT | |
| | | TGGTTGGGGTGACAAGGATAAAATATAGAATAACTGTCTT | |
| | | TTTTTTTTACAGCAATATTTGGTTTAATGATCCTAAAAAG | |
| | | GATTAAAAGATTAAGTTACTTTAGGGATAACAGCGTGAT | |
| NC_02 | Homarus | TAAAGGGACGATAAGACCCTATAAAGCTTAATAATTTAGT | 582 |
| 0020 | gammarus | ATATAATTAGATGAGTTAAAAGTTTAATATTATTTATATA | |
| | | CTGAATTATTTCGTTGGGGCGACGATGATATAATTTGTAA | |
| | | CTGTTTAAATCTAAAATACAGAGATATTTGTGTGTAATGA | |
| | | TCCTTGTTGTTGATTAAAAATTTAAGTTACTTTAGGGATA | |
| | | ACAGCGTTAT | |
| NC_02 | Thenus | AGGGGACGATAAGACCCTATAAATCTTTATATTAATTTGT | 583 |
| 4440 | orientalis | TGTTAAATTGGAAAGTAGAATTAATTCCGGGTTTAATAAT | |
| | | TTTGAATATTTTGTTGGGGAGACAGAAGTGTAAATAAATA | |
| | | ACTACTTTAAAAAAAAAAGTTAATGATATTTAATGAGGAG | |
| | | ATCCTTTACTAAGGATTGTAGAATAAGTTACTTTAGGGAT | |
| | | AACAGCGTAAT | |
| NC_02 | Lithodes | CAAAAAGACGATAAGACCCTATAAATCTTTACAATAAATA | 584 |
| 4202 | nintokuae | TATTTTATATTTTAGCTTATAAGTGTATAAGAAATAAAAA | |
| | | TATAGATTTGTTGCGCTGGGGCGGCGTAGATATATAAATA | |
| | | AACTGTCTATAGTTTAAATACAATAATCATTGCTTAATAC | |
| | | AAATTGATCCTTAAATAGATTAAAAGATTAAGATACTTTA | |
| | | GGGATAACAGCGTTAT | |
| NC_02 | Cherax | GGGGGACGATAAGACCCTATAAAGTTTGTACACTAAACTA | 585 |
| 3481 | cairnsensis | GTTTAGAGTGATTTAGGTTTATAAAGTTTACTATTGGGGA | |
| | | GGTGTTTAGTTGGGGCGACTAGGATATAAGTTATCTAACT | |
| | | GTTTCTTTGTTGTAATCAGAAATATTTGAGTTTATGATCC | |
| | | TTTTTTAGGATTATAGAGTAAATTACTTTAGGGATAACAG | |
| | | CGTAAT | |
| NC_02 | Cherax | GGGGGACGATAAGACCCTATAAAGTTTGTACATTAAACTA | 586 |
| 3480 | dispar | ATTAAAGGTGGTTTAGGTTTATAAAATTTGCCATTAGGGA | |
| | | AGTGTTTAGTTGGGGCGATTGGGATATAAGTTATCTAACT | |
| | | GTTTCTTTGTTTTAATCAAAAATATTTGAGTTTATGATCC | |
| | | TTTTTTAGGATTAAAGAGTAAATTACTTTAGGGATAACAG | |
| | | CGTAAT | |
| NC_02 | Cherax | AAGGGACGATAAGACCCTGTAAAGTTTATACATTAAGTTA | 587 |
| 3479 | quinquec | ATTAAAAATAATTAAGGAGATTAAAATTTATTATTGGGTA | |
| | arinatus | GATGTTTAGTTGGGGCGACTAGGATATAATTTAATTATAA | |
| | | CTGTCTTAGATTAAGGTCAAAGATATTTGAGTTTGTGATC | |
| | | CTTTCTTAGGATTCAAGAACAAGTTACTTCAGGGATAACA | |
| | | GCGTAAT | |
| NC_02 | Cherax | GAGGGACGATAAGACCCTATAAAGTTTTGTACATTAAACC | 588 |
| 3478 | robustus | AATTAATAGGCGATTTAGGTTTGTAAGGTTTACTATTAGG | |
| | | TAGATGTTTGGTTGGGGCGACTAGGATATAAGTTATTTAA | |
| | | CTGTTCTTTGTTAGAATCAAAGGTATTTGAGTTTATGATC | |
| | | CTTTTTAAGGGTTATAGAATAAATTACTTTAGGGATAACA | |
| | | GCGTAAT | |
| NC_02 | Cherax | GAGGGACGATAAGACCCTATAAAGTTTGTACACAGAGTTA | 589 |
| 2938 | monticola | ATTAAGAGCGATTTGGAGTAATAAAATTTATTATTAGCAA | |
| | | GGTGTTTGGTTGGGGCGACTAGAATATAAGTTATATAACT | |
| | | GTTTTTTGTTTAAATCAGAGATATCTGTTTATATGATCCC | |
| | | TTTTTAGGATTAAAGAGTAAATTACTTTAGGGATAACAGC | |
| | | GTAAT | |
| NC_02 | Cherax | AAGGGACGATAAGACCCTATAAAGTTTATATACAGAACTA | 590 |
| 2939 | glaber | ATTAAAGATAATTAGGAAGAATAAAATTTATTATTAGGTA | |
| | | GGTATTTAGTTGGGGCGACTAGGATATAAATTAATTTAAC | |
| | | TGTTTTTAATTAAGGTCAAAGATATTTGAGTTCGTGATCC | |
| | | TTTTTTAGGATTAAAGAATAAGTTACTTTAGGGATAACAG | |
| | | CGTAAT | |
| NC_02 | Cherax | GAGGGACGATAAGACCCTATAAAGTTTGAACACCGGGTTT | 591 |
| 6224 | holthuisi | GTTAAGAGTGATTTAGAATAGTAAAGTTTGTTACTAGTAA | |
| | | AGTGTTTGGTTGGGGCGACTAGAATATAAGTTATATAACT | |
| | | GTTTTTTATTTGAATCAGAGATATCTGTTTATATGATCCC | |
| | | TTTTTAGGATTAAAGAGTAAATTACTTTAGGGATAACAGC | |
| | | GTAAT | |
| NC_02 | Astacopsis | GGGGGACGATAAGACCCTATAAAGTTTAACATAATGAGGA | 592 |
| 6215 | gouldi | TAAAAAATTAATTAGATTATAAAGTTTATTATCATAAATA | |
| | | ATGTTTTGTTGGGGCGACAAGAATAAAAATAATTTAACTG | |
| | | TTCTTTTTTAGATACAAAAATATTTGTGTAGGGTGATCTT | |
| | | TTTTAGAAGTATTAGAGTAAATTACTTTAGGGATAACAGC | |
| | | GTAAT | |
| NC_02 | Portunus | GAGGGACGATAAGACCCTATAAAGCTTTATAAATTTAAAG | 593 |
| 6209 | pelagicus | ATTTTATATATTTTTTATTATAATGATAATTTCTCAAAAA | |
| | | TTTATTGGGTTGGGGCAACAAAAGTATAATTATCAATAAC | |
| | | TGCTATAAATTACTACAATTATTTTTGTTATTAAAGAGTT | |
| | | GTTGATCCTCGAAAGGGATTAAAAGATCAAGTTACTTTAG | |
| | | GGATAACAGCGTAAT | |
| NC_02 | Paranephrops | AAGGGACGATAAGACCCTATAAAGCTTTACATTGAATTTA | 594 |
| 5957 | planifrons | TTAAAAAGTAATTTAAATAATAAAAGTTTATTAGTAGGGA | |
| | | GATGTTTTGTTGGGGTGACAAGAATATAATAAATATAACT | |
| | | GTTCTTTTTTATTTCAAAAATATTTGAATGGGAGATCCTT | |
| | | AATAAAGATGTTAGAGTAAGTTACTTTAGGGATAACAGCG | |
| | | TAAT | |
| NC_02 | Nephrops | TAAAGGGACGATAAGACCCTATAAAGCTTAATAATTTAAT | 595 |
| 5958 | norvegicus | ATATAACCAGATAAATTAAAAGTTTAATATTCTTTATATA | |
| | | TTAAATTATTTCGTTGGGGCGACGATGATATAATTTGTAA | |
| | | CTGTTTAAATTTTAAATACAGAGATATTTGTGTGTAATGA | |
| | | TCCTTTTTATTGATTAAAAATTTAAGTTACTTTAGGGATA | |
| | | ACAGCGTTAT | |
| NC_02 | Ibacus | AGGGGACGATAAGACCCTATAAATCTTTATATTTTATATT | 596 |
| 5581 | ciliatus | TATTAGTTTAAAAATTGGTATAAAATTTTGATTTAATAAT | |
| | | TTAAAATATTTTGTTGGGGAGACAGGAGTATAATAAATAA | |
| | | CTGCTTTAAAATTTAAGTTAATAATATTTAATTGAGTGAT | |
| | | CCTTTAATATGGATTTTAGATTAAGTTACTTTAGGGATAA | |
| | | CAGCGTAAT | |
| NC_02 | Charybdis | GAGGGACGATAAGACCCTATAAAGCTTTATAAATTTAAAA | 597 |
| 4632 | feriata | ATCTTATATATTTTTATATAAAAATAATTTTTGAGGAGTT | |
| | | TATTTGGTTGGGGCGACAGTGGTATAAGTAATGTAACTGC | |
| | | TATAGTGTTTAACAAAAATATTTGTTTAGTGTAAGTAATT | |
| | | GATCCTTTATAAGGATTTTAAGATCAAGTTACTTTAGGGA | |
| | | TAACAGCGTTAT | |
| NC_02 | Metaneph | GAGGGACGATAAGACCCTATAAAGCTTGATAATTTTGTAT | 598 |
| 5323 | ropssibogae | ATAAATAAATAAGTTGTTAGTATTATACTGTCTACATATA | |
| | | AAATTATTTCGTTGGGGCGACGATAATATAATCTGTAACT | |
| | | GTTTAGGTTTTAACTCAAATATATTTGTGTTTAATGATCC | |
| | | ATTAATTGTTGATTAAAAATTTAAGTTACTTTAGGGATAA | |
| | | CAGCGTTAT | |
| NC_02 | Panulirus | GGGGGACGATAAGACCCTATAAATCTTGATGATCGGGGTA | 599 |
| 8024 | cygnus | GTAATTTTTATGTAGTAGTTATAAACTTAAATTTAATACC | |
| | | TTGATTATTTTGTTGGGGCGACAGGGGTATAATTAGTAAC | |
| | | TGCCTTGGATAAACAGCTAATTATTTTTATTGTGTTATGT | |
| | | CTAAGATCCTTCTTTGAAGATATCAGATCAAGTTACTTTA | |
| | | GGGATAACAGCGTAAT | |
| NC_02 | Metanephrops | GAGGGACGATAAGACCCTATAAAGCTTGATAATTTTATAT | 600 |
| 7608 | thomsoni | ATAAATAAATAAGTTGTTAGTGTTATATCATCTATTTATA | |
| | | AAATTATTTCGTTGGGGCGACGATAATATAATTTGTAACT | |
| | | GTTTAAGATTTAGATTCAAATATATTTGTGTTTTAATGAT | |
| | | CCATTAGTTGTTGATTAAAAATTTAAGTTACTTTAGGGAT | |
| | | AACAGCGTTAT | |
| NC_02 | Faxonius | TTGGGACGATAAGACCCTATAAAACTTTATATTTTAAGGT | 601 |
| 6561 | limosus | AGGAGTTAATTTCATTTTAAGGTTACTATTTTAAAATGTT | |
| | | TTGTTGGGGGGACAAGGATATAAGAAATGATAACTGTCTT | |
| | | TTATTTTTACAATAATATTTGATTTATTGATCCTGAAAGG | |
| | | GATTTAAGATTAAGTTACTTTAGGGATAACAGCGTAAT | |
| NC_02 | Squilloides | GAGGGACGATAAGACCCTATAAAGCTTTATATTATATTTC | 602 |
| 7178 | leptosquilla | TTTAATATAAATTGTAATGTTATTAACTTATTTTAGAAAA | |
| | | TATATTATTTTACTGGGGGGGTAAAAGTATATTAAAGATT | |
| | | AACTGCTTTTGTTTTGTATAACAATTATAGTTGAAAATAA | |
| | | AATGATCCATTATTAATGATCATAAAACCAAGTTACTTTA | |
| | | GGGGTAACAGCGTAAT | |
| NC_02 | Cherax | GAGGGACGATAAGACCCTATAAAGTTTATACATGGGGCTG | 603 |
| 6226 | bicarinatus | GTTGAGAGTGATTCAGAATAGTAAAGTTTATTATTAGCAA | |
| | | GATGTTTAGTTGGGGCGACTAAGATATAAATTATATAACT | |
| | | GTCTTTTGTTTAAATCAGAGGTATCTGTTCGTATGATCCT | |
| | | TTTTTAGGATTGGAGAGTAAATTACTTTAGGGATAACAGC | |
| | | GTAAT | |
| NC_02 | Austropot | GGGGGACGATAAGACCCTATAAAACTTTATATTTTAAAAA | 604 |
| 6560 | amobius | TATTAACTAGTTTTGTATAAGAGGGTTTTTTTTTGAGTAT | |
| | pallipes | TTTATTGGGGTGATAGGGATATAATAAAAGATAACTGTCT | |
| | | CTTTTTTTTTACAGAGATATTTGAGTAAATGATCCTAATA | |
| | | AGGGAAAGAAGGTTAAGTTACTTTAGGGATAACAGCGTAA | |
| | | T | |
| NC_02 | Cherax | AAGGGACGATAAGACCCTATAAAGTTTATACATTCAAATA | 605 |
| 6559 | tenuimanus | ATTGAAAATAATTAAGAAAAATTAAAGTTTATTATTAGGA | |
| | | AGATGTTTAGTTGGGGCGACTAGGATATAAGTTAATTTAA | |
| | | CTGTCTTAAATTAAAGTCAAAGATATTTGAGTTTGTGATC | |
| | | CTTTTTTAGGATTCAAGAACAAGTTACTTTAGGGATAACA | |
| | | GCGTAAT | |
| NC_02 | Cherax | GAGGGACGATAAGACCCTATAAAGTTTATACATGGAGTTA | 606 |
| 6227 | boesemani | GTTAAGAGTGATTTAGGATAGTAAAGTTTGTTATTAGCAA | |
| | | GATGTTTGGTTGGGGCGACTAGAATATAAGTTATATAACT | |
| | | GTTTTTTATTTAAGTCAGAGGTATTTGTTTATAGTGATCC | |
| | | CTTTTTAGGATTAAAGAGTAAATTACTTTAGGGATAACAG | |
| | | CGTAAT | |
| NC_03 | Charybdis | GAGGGACGATAAGACCCTATAAAGCTTTATAAATTTAAAG | 607 |
| 6132 | (Charybdis) | ATTTTGTATGTTTTTATATAAAGATAAATTCTAAAGAATT | |
| | natator | TATTGGGTTGGGGCGACAATGGTATAATGAAAATAACTGC | |
| | | TATTAAAATTAACAACTATATTTGGATTAAGTTAAATAAT | |
| | | TGATCCTTTATAAGGATTTTAAGATCAAGTTACTTTAGGG | |
| | | ATAACAGCGTTAT | |
| NC_03 | Procambarus | AAGGGACGATAAGACCCTATAAAACTTTATATTTATAAGA | 608 |
| 3510 | acutus | TAGTAGCTAGTTTTATTTAAAAGTATTATTTTGGAGTATT | |
| | | TGGTTGGGGTGACAAGGATAAAATATAAAATAACTGTCTT | |
| | | TTTTTTTTACAGTAATGTTTGGTTTAATGATCCTAAAAGG | |
| | | GATTAAAGATTAAGTTACTTTAGGGATAACAGCGTAAT | |
| NC_03 | Pacifastacus | GAGTGACGATAAGACCCTATAAAACTTTATATTTTAAGAT | 609 |
| 3509 | leniusculus | AATAGTTAGTTTTATTTAAGAGTTTTATTTTGAAATATTT | |
| | | TATTGGGGTGATAAAGATATAAATTAAAATAACTGTCTTT | |
| | | TTTTTTTACAATAATCTTTGAATTAATGATCCTAAGAAAG | |
| | | GAGTAAAAGATCAAGTTACTTTAGGGATAACAGCGTAAT | |
| NC_03 | Munida | AAGGGACGATAAGACCCTATAAATCTTTATATTAAATTAT | 610 |
| 0255 | gregaria | TTTTAGTTAATTAATAATGTAATTTTAACAGTAAAATAAA | |
| | | ATATATTGTGTTGGGGTGATGAGAATATAAAATTAACTGT | |
| | | TCTAAATTGAAAACAAATTTATTTGAATTAATAATAGATC | |
| | | CTTAATAGAGATTAAAAGTTTAAGTTACTTTAGGGATAAC | |
| | | AGCGTTAT | |
| NC_02 | Panulirus | GGGGGACGATAAGACCCTATAAATCTTTATGCTTTAATTA | 611 |
| 8627 | versicolor | ATAAATCTTAGAAATTAGTTACAAATCTAGCGTCATTGAG | |
| | | TTAAAGTATTTTGTTGGGGCGACAGAAGCATAATAAGTAA | |
| | | CTGCTTTCGTTTAAAAAAATTAATTATTTTTATTGTGATA | |
| | | GTTATATTGATCCTCTTATGGAGAAACCAGATCAAGTTAC | |
| | | TTTAGGGATAACAGCGTAAT | |
| NC_02 | Faxonius | ATGGGACGATAAGACCCTATAAAACTTTATATTTTAATGT | 612 |
| 9720 | rusticus | AGAAGTTAATTTTATTTTAAGTTCACTATTTTAAAATTTT | |
| | | TTGTTGGGGCGACAAGGATATAAAAAATGATAACTATCTT | |
| | | TTATTTTTACAATAATATTTGATTTATTGATCCTAAAAGG | |
| | | GATTAAAGATTAAGTTACTTTAGGGATAACAGCGTAAT | |
| NC_02 | Portunus | GAGGGACGATAAGACCCTATAAAGCTTTATAAATTTAAAG | 613 |
| 8225 | sanguinolentus | ATTTTACATATTTTATCTATAATGGTAATCTCTGGAGAAT | |
| | | TTATTGGGTTGGGGCAACAAAAGTATAATTATCAATAACT | |
| | | GCTATAAAATAATACAATTATCTTTGTGTGGGTAGATTAG | |
| | | TTGATCCCTGAAAAGGATTAAAAGATCAAGTTACTTTAGG | |
| | | GATAACAGCGTAAT | |
| NC_02 | Procambarus | AAGGGACGATAAGACCCTATAAAACTTTATATTTATAATA | 614 |
| 8447 | alleni | TAGTAGTTAGTTTTATTTAATAATATTATATTAAAGTATT | |
| | | TGGTTGGGGTGACAAGGATATAATATAAAATAACTGTCTT | |
| | | TTTTTTTTACAGTAATATTTGGTTTAATGATCCTAGAAGG | |
| | | GATTAAAAGATTAAGTTACTTTAGGGATAACAGCGTAAT | |
| NC_05 | Metacarcinus | GGGGGACGATAAGACCCTATAAAGCTTTATATTATCAGCT | 615 |
| 0675 | magister | ATGATTTAATTAAATTAAAAAGAAATTAAAGGTTTTAATA | |
| | | AGCTATTATATTTTATTGGGGAGATATAAGTATAATTTTA | |
| | | TGTAACTGCTTATAAGTCGATACATTGATAATTGGTTTAA | |
| | | ATTTTAATGATCCTTTGAATAAAGATTCAAGACCAAGTTA | |
| | | CTTTAGGGATAACAGCGTTAT | |
| NC_04 | Puerulus | GGGGGACGATAAGACCCTATAAATCTTTATATTTGCTCAG | 616 |
| 1155 | angulatus | CCAAAAAATGTGTATTAGGGCCAAAAATATATTTTTTGGT | |
| | | GGGGTGATATTTTGTTGGGGCGACAGGAGTATAATAAGTA | |
| | | ACTGCTTTAAAAGTTTAATTAGATATATTTAGTTTGAGTG | |
| | | TTAAAAGATCCTTTAGTAAGGATTATTAAAATAAGTTACT | |
| | | TTAGGGATAACAGCGTAAT | |
| NC_04 | Lupocycloporus | GAGGGACGATAAGACCCTATAAAGCTTTATAAATTCGATA | 617 |
| 0124 | gracilimanus | ATTTTGCAAATTTGTACAAATAAAAGTAATTTTGAAAAAT | |
| | | TTATTTGGTTGGGGCGACAAAAGTATAATTAAAATAACTG | |
| | | CTAGAATTTCAAACAATAATTTTTGATTTAGGTTTGTAAA | |
| | | TGATCCCTTATAAGGATTAAAAGATCAAGTTACTTTAGGG | |
| | | ATAACAGCGTAAT | |
| NC_03 | Monomia | TAAGGGGACGATAAGACCCTATAAAGCTTTATAAATTCAG | 618 |
| 7173 | gladiator | AGATTTTAGATATTTTATATATAAATTTATTTTTTGATAA | |
| | | TTTATTTGGTTGGGGCGACAAGGGTATAATTATAAATAAC | |
| | | TGCCATTAATGTTTATACATATATTTTTGATTTAAATTTA | |
| | | ATATAGTAGTTGATCCTTTTTAAGGATTTAAAGATCAAGT | |
| | | TACTTTAGGGATAACAGCGTAAT | |
| NC_03 | Varuna | TTAAAGTGACGATAAGACCCTATAAAGCTTTATATAGATA | 619 |
| 7155 | yui | TATTATTTAATTAAATTTTTAATAAAAATTTAGTAGTAAA | |
| | | ATTTATTTTATTGGGGCGATAAGAGTAAAATTTTTATTAA | |
| | | CTGCTTTAAGTTTTGAATACATTTATGTTTGATTAAAATT | |
| | | TTAAATGATCCTAAATAAAGATTAAAAGTTTAAGTTACTT | |
| | | TAGGGATAACAGCGTTAT | |
| NC_03 | Panulirus | GGGGGACGATAAGACCCTATAAATCTTTACAATTAGGGCT | 620 |
| 9671 | argus | TTAGTTATTAGAAAGTAGTTATGGATCTAAATCTAAGGTT | |
| | | TAAATTGTTTTGTTGGGGCGACAGAAGTATAATAAGTAAC | |
| | | TGCTTTGGTTTTCATATTAATTGTTTTTATTATGGTTAGT | |
| | | TTAAAAGATCCTCTTTTGGAGATCACAGACTAAGTTACTT | |
| | | TAGGGATAACAGCGTAAT | |
| NC_03 | Munida | AAGGGACGATAAGACCCTATGAATCTTTATATTATGTTAC | 621 |
| 9112 | isos | TTTTAATTAATTAATGTTGTAGATATAGTGATAAGGTGGG | |
| | | GTATATTGTGTTGGGGTGATGAGAATATAAGAAATAACTG | |
| | | TTCTAAATTTAAAACAAAATTGTTTGAGGTAATAATAGAT | |
| | | CCTTTATTGAGATTAAAAGTTTAAGTTACTTTAGGGATAA | |
| | | CAGCGTTAT | |
| NC_04 | Scyllarides | GAGGGACGATAAGACCCTATAAATCTTTATACTTAGGTTT | 622 |
| 4425 | squammosus | ATACGGTTGAAGGTTAGGTTACTATTTAATTTTATAAAGT | |
| | | TAAGTATTTTGTTGGGGTGACAGGGGCATAAGAGTGAATA | |
| | | ACTGCCTTAGAGTTAGAGTTAAAAGTATTTAATTTATTAA | |
| | | GTGATCCTTTAGTAGAGAATATAGACTAAGTTACTTTAGG | |
| | | GATAACAGCGTAAT | |
| NC_01 | Cambaroi | GGGGGACGATAAGACCCTATAAAACTTTATATACTTATAA | 623 |
| 6925 | dessimilis | TTTAATAATAACTTTTGGGTTTAAAAATTTAGATTAGAGT | |
| | | ATTTTATTGGGGCGATAAAGATAAAATTTTAGATAACTGT | |
| | | TTTATTTTTTAACAAAAATGTTTGAGTTTTAGATCCTTAA | |
| | | AGAGATTAAAAGATTAAGTTACTTTAGGGATAACAGCGTT | |
| | | AT | |
| NC_03 | Charybdis | GAGGGACGACAAGACCCTATAAAGCTTTATAAATTTGATA | 624 |
| 7695 | bimaculata | ATTTTTTAAATTTATATATAAAAATAATTTTTGTAAAATT | |
| | | TATTTGGTTGGGGCGACAATGGTATAATTAAGATAACTGC | |
| | | TATAAGATTTAACAAAAATATTTGTTTAATAAATATAATT | |
| | | GATCCTTTTTAAAGATTTAAGATCAAGTTACTTTAGGGAT | |
| | | AACAGCGTTAT | |
| NC_03 | Cambarus | AAGGGACGATAAGACCCTATAAAACTTTATATTTAGAAAT | 625 |
| 3507 | robustus | AATGGGTAAGTTTTGTTTAGAAGTTATTATTTTAGAGTAT | |
| | | TTGGTTGGGGCGACAAGGATAAAAAGAAGATAACTGTTCT | |
| | | TTTATTTTACAATAATGTTTGGGTATAATGATCCTAATAT | |
| | | GGGTTAAAAGATTAAGTTACTTTAGGGATAACAGCGTAAT | |
| NC_03 | Thalamitasima | GAGGGACGATAAGACCCTATAAAGCTTTATAAACTTATAA | 626 |
| 9640 | | ATTTTATATGTTTTTTTATAAAAATAATCTTTCGAAGGTT | |
| | | TATTTAGTTGGGGCGACAATGATATAAATAATATAACTGT | |
| | | CATAAAATATAACAAATATATTTGTTTAATAAGGAATAAT | |
| | | TGATCCTTTATAAGGATTTTAAGATCAAGTTACTTTAGGG | |
| | | ATAACAGCGTTAT | |
| NC_02 | Thranita | AAGGGACGATAAGACCCTATAAAGCTTTATAATTTTAAAG | 627 |
| 4438 | crenata | ATTTTATATATTTTTCTATAGTAATAATTTCTTGAAAACT | |
| | | TATTTGGTTGGGGCGACAATAGTATAATTAGAATAACTGC | |
| | | TTAAAATTAATACAAATATATCTGAGTTAAAATTGACTGA | |
| | | TCCTTTATAAAGATTTTAAGATCAAGTTACTTTAGGGATA | |
| | | ACAGCGTTAT | |
| NC_03 | Orconectes | AGGGGACGATAAGACCCTATAAAACTTTATATTTTAAGAT | 628 |
| 3508 | luteus | GGAGATAAATTTTATTTTAAGGTTACTATTTTAAATTGTT | |
| | | TTGTTGGGGCGACAAGGATATAAGGAAAGGATAACTGTCT | |
| | | TTTGTTCTTACAATAATATTTGAATTATTGATCCTAAAAG | |
| | | GGATTAAAGATTAAGTTACTTTAGGGATAACAGCGTAAT | |
| NC_03 | Orconectes | AAGGGAAGATAAGACCCTATAAAACTTTATATTTTAAGGT | 629 |
| 0768 | punctimanus | AGAAATTAATTTTATTTTTAGATTACTATTTTAAAATATT | |
| | | TTGTTGGGGTGACAAGGATATAATAAAAGGTAACTGTCTT | |
| | | TTGGTTTAACAATAATATTTGGGTAATTGATCCTAAAATG | |
| | | GATAAAAGATTAAGTTACTTTAGGGATAACAGCGTAAT | |
| NC_02 | Orconectes | TTGGGACGATAAGACCCTATAAAACTTTATATTTTAAGGT | 630 |
| 9721 | sanbornii | AGAAATTAATTTTATTTTAAGGTTACTATTTTAAAATGTT | |
| | | TTGTTGGGGTGACAAGGATACAAGAAATGATAACTGTCTT | |
| | | TTATTTTTACAATAATATTTGATTTATTGATCCTGAAAGG | |
| | | GATTAAAGATTAAGTTACTTTAGGGATAACAGCGTAAT | |
| NC_02 | Cherax | AAGGGACGATAAGACCCTATAAAGTTTATATACCGAACTA | 631 |
| 4029 | crassimanus | ATTAAAGATAATTAAGAAGGGTAAAGTTTATTATTAGAGA | |
| | | GGTATTTAGTTGGGGCGACTAGGATATAAATTAATTTAAC | |
| | | TGTTTTAAATTAAAGTCAAAAATATTTGAGTTTGTGATCC | |
| | | TTTTTTAGGATTAGAGAATAAGTTACTTTAGGGATAACAG | |
| | | CGTAAT | |
| NC_02 | Cherax | AAGGGACGATAAGACCCTATAAAGTTTATATGTTGAACTA | 632 |
| 3482 | preissii | ATTAAAGATAATTAGGAAGAGTAAAGTTTATTATTGGTTA | |
| | | GGCATTTAGTTGGGGCGACTGAAATATAAGTTAATTTAAC | |
| | | TGTTTTAAATTAAGGTCAAAGATGTTTGAGTTTGTGATCC | |
| | | CTTTTTAGGATGATAGAATAAGTTACTTTAGGGATAACAG | |
| | | CGTAAT | |
| KF051 | Munida | AAGGGACGATAAGACCCTATGAATCTTTATATTATGTCAC | 633 |
| 309 | spinosa | TTTAAGTTAGTTAATATCGTAGATATAATGATAAGGTGAG | |
| | | GTGTATTGTGTTGGGGTGATGAGAATATAAGAAATAACTG | |
| | | TTCTAAATTTAAGACAAAATTGTTTGTGGTAATAATAGAT | |
| | | CCTTTATTGAGATTAAAAGTTTAAGTTACTTTAGGGATAA | |
| | | CAGCGTTAT | |
| MK847 | Munida | AAGGGACGATAAGACCCTATAAATCTTTATATTAGATTAT | 634 |
| 971 | asprosoma | TTTTAGTTATTTAATATTGTGAGTATAACAATAAAGTGGT | |
| | | ATATATTGTGTTGGGGTGATGAGAATATAATAAATAACTG | |
| | | TTCTAAAATTAAAACAAAATTATTTGTATAAATAATAGAT | |
| | | CCTTTATTGAGATTAAAAGTTTAAGTTACTTTAGGGATAA | |
| | | CAGCGTTAT | |
| MK847 | Munida | AAGGGACGATAAGACCCTATAAATCTTTATATTAAATTAT | 635 |
| 969 | leagora | TTTTAGTTGATTAATATTGTAAATATAATAATAAAATATC | |
| | | AAATATTGTGTTGGGGCGACGAGAATATAATAAATTAACT | |
| | | GTTCTAAAATTAAAACAAAATTGTTTGTAAAAATAATAGA | |
| | | TCCTTTATAAAGAGTAAAAGTTCAAGTTACTTTAGGGATA | |
| | | ACAGCGTTAT | |
| AY351 | Munida | AAGGGACGATAAGACCCTATAAATCTTTATATTAAATTGT | 636 |
| 105 | alonsoi | TTAATATTTAATTAATATTGTGTACTAGATATTTAAATAA | |
| | | TATATATTGTGTTGGGGTGACGAGAATATAATAAATAGCT | |
| | | GTTCTAAAGTAAAAACAATTTTTATTGTGGTAATAGAAGA | |
| | | TCCTTTATTGAGATTGAAAGTTTAAGTTACTTTAGGGATA | |
| | | ACAGCGTTAT | |
| AY351 | Munida | AAGGGACGATAAGACCCTATAAATCTTTATATTAAATTAT | 637 |
| 177 | taenia | TTTCAATTAATTAATACTGTAAATACAATAATAAAATAAT | |
| | | AAATATTATGTTGGGGCGATGAGAATATAATAAATAACTG | |
| | | TTCTAAAATTTATACAAAATTATTTGTTACAATAATAGAT | |
| | | CCTTTATAGAGATTAAAAGTTTAAGTTACTTTAGGGATAA | |
| | | CAGCGTTAT | |
| MK847 | Munida | GAGGGACGATAAGACCCTATAAATCTTTATATTAAGTTGT | 638 |
| 957 | gordoae | TTTTTACTAATTAATATTGTAGATGAAGTAATAAAATTAT | |
| | | ATATATTGTGTTGGGGCGATGAGAATATAAATAAATAACT | |
| | | GTTCTAGTGTAGAAAAACAAAATTATTTGGATATTGGTAG | |
| | | ATCCTTTAGTTAAGATTAAAAGTTTAAGTTACTTTAGGGA | |
| | | TAACAGCGTTAT | |
| AY351 | Munida | AAGGGACGATAAGACCCTATAAATCTTTATACTAGGATAT | 639 |
| 190 | zebra | TTTTAGTTAATTAATATTGTAAATATAATAATAAAATGAC | |
| | | ATGTATTGTGTTGGGGTGATGAGAATATAATAAATAACTG | |
| | | TTCTAAGATGAAAAAAAATTATTTGTAGTAATAATAGATC | |
| | | CTTTATTGAGATTGTAAGTTTAAGTTACTTTAGGGATAAC | |
| | | AGCGTTAT | |
| AY351 | Munida | AAGGGACGATAAGACCCTATAAATCTTTATATTAAACTGT | 640 |
| 119 | distiza | TTTCAATTAATTAATATTGTTAATATAATAGTAAAAGAGT | |
| | | AAATATTATATTGGGGCGATAAGAATATATAAATTAACTG | |
| | | TTCTAAAGTTAAAACAAAATTATTTGTAAAAATAATAGAT | |
| | | CCTTAATAAAGATTAAAAGTTCAAGTTACTTTAGGGATAA | |
| | | CAGCGTTAT | |
| AY351 | Munida | AAGGGACGATAAGACCCTATAAATCTTTATATTAAGTTAT | 641 |
| 158 | psamathe | TTTTAGTTAATTAATATTGTAAATGAAATTACAAAATAAT | |
| | | ATATATTGTGTTGGGGTGATGAGAATATAATAAATATCTG | |
| | | TTCTAAAATAAAAACAATTTTTTTGTGTTAGTAGAAGATC | |
| | | CTTTGTTGAGATTAAAAGTTTAAGTTACTTTAGGGATAAC | |
| | | AGCGTTAT | |
| AY351 | Munida | GAGGGACGATAAGACCCTATAAATCTTTATATTAGGTTAT | 642 |
| 181 | thoe | TTTATAGTTAGTTAATATTGTAAATATAATAATAAGGTGA | |
| | | AATATATTATGTTGGGGTGATGAGAATATAATAAATAACT | |
| | | GTTCTAAGATAAAAACAAAATTGTTTGTAAAATAGTAGAT | |
| | | CCTTTGTTGAGATTAGAAGTTTAAGTTACTTTAGGGATAA | |
| | | CAGCGTTAT | |
| AY351 | Munida | AAGGGACGATAAGACCCTATAAATCTTTATATTAAACTGT | 643 |
| 130 | guttata | TTTCAATTAATTAATATTGTTAATATAATAGTAAAATAGT | |
| | | AAATATTATATTGGGGTGATAAGAATATATAAATTAACTG | |
| | | TTCTAAAATTAAAACAAAATTATTTGTAGTAATAATAGAT | |
| | | CCTTAATAAAGATTAAAAGTTCAAGTTACTTTAGGGATAA | |
| | | CAGCGTTAT | |
| AY351 | Munidastia | AAGGGACGATAAGACCCTATAAATCTTTATATCAAATTAT | 644 |
| 170 | | TTTTAGTTGATTAATATAGTAGATATTAATAATAAAATAA | |
| | | CACATATTATATTGGGGTGATAAGAATATAAAAAATAACT | |
| | | GTTCTAAAATTAAAACAAAATTATTTGTAGTAGTTATAAT | |
| | | AGATCCTTTATTGAGATCCAAAGTTTAAGTTACTTTAGGG | |
| | | ATAACAGCGTTAT | |
| AY351 | Munida | AAGGGACGATAAGACCCTATAAATCTTTATATTAAGTTAA | 645 |
| 150 | ommata | TTTTTTAGTTAATTAATATTGTAAATATAATAGTAAGATG | |
| | | GCGTATATTGTGTTGGGGTGATGAGAATATAATAAATAAC | |
| | | TGTTCTAAAATATAAACAATATTGTTTGTAAATAATAGTA | |
| | | GATCCTTTATTGAGATTAAAAGTTTAAGTTACTTTAGGGA | |
| | | TAACAGCGTTAT | |
| MK847 | Munida | AAGGGACGATAAGACCCTATAAATCTTTATATTAAACTAT | 646 |
| 958 | roshanei | TTAAAATTAATTAATATGTAAATTTAATAAATAAAATGGA | |
| | | AAATATTATATTGGGGTGATAAGAATATAAATTATATAAC | |
| | | TGTTCTAAAATTTAAACAAAATTATTTGTACAAATAGTAG | |
| | | ATCCTTTTTTAAGATTAAAAATTTAAGTTACTTTAGGGAT | |
| | | AACAGCGTTAT | |
| MK847 | Munida | AAGGGACGATAAGACCCTATAAATCTTTATATTAGATTAT | 647 |
| 944 | compressa | TTTTAAGTTATTTAATATTGTAAATATAATAATAAAATAA | |
| | | TATATATTGTGTTGGGGTGATGAGAATATAATGAATAACT | |
| | | GTTCTAAAATGAAAACAAAATTATTTGTGAAAATAATAGA | |
| | | TCCTTTATTGAGATTAAAAGTTTAAGTTACTTTAGGGATA | |
| | | ACAGCGTTAT | |
| AY351 | Munida | AAGGGACGATAAGACCCTATAAATCTTTATATTAAACTAC | 648 |
| 113 | clinata | CTTAAATTAATTAATATTGTAAATAATAACGGTAAGGTAG | |
| | | AAAATATTATATTGGGGTGATAAGAATATAAAATAATATA | |
| | | ACTGTTCTAAAATTTAAACAAAATTATTTGTAATAATAAT | |
| | | AGATCCTTTTTTAAGATCAAAAATTTAAGTTACTTTAGGG | |
| | | ATAACAGCGTTAT | |
| MK847 | Munida | GAGGGACGATAAGACCCTATAAATCTTTATATTAAATTAT | 649 |
| 964 | chydaea | TTTTAGTTAATTAATATTGTGAACATAATAATAAAGTAAG | |
| | | GAAATATTGTGTTGGGGTGATGAGAATATAAATAATAACT | |
| | | GTTCTAAAATTAAAACAAAATTATTTGTAAGTAATAATAG | |
| | | ATCCTTTATTGAGATTAAAAGTTTAAGTTACTTTAGGGAT | |
| | | AACAGCGTTAT | |
| MK847 | Munida | AAGGGACGATAAGACCCTATAAATCTTTATATTAGATTAT | 650 |
| 941 | compacta | TTTTAGTTGTTTAATATTGTAAATATAATAATAAAATGAT | |
| | | ATATATTGTGTTGGGGTGATGAGAATATAATAAATAACTG | |
| | | TTCTAAAATGAAAACAATATTATTTGAATGAATAGTAGAT | |
| | | CCTTTATTGAGATTAAAAGTTTAAGTTACTTTAGGGATAA | |
| | | CAGCGTTAT | |
| AY351 | Munidaeclepsis | AAGGGACGATAAGACCCTATAAATCTTTATATTAGATTAT | 651 |
| 122 | | TTTTAGTTATTTAATATTGTAAATATAATAATAAAGTGGT | |
| | | GTATATTATGTTGGGGTGATGAGAATATAATAAATAACTG | |
| | | TTCTAAAATAAAAACAAAATTGTTTGTATAGATAATAGAT | |
| | | CCTTTATTGAGATTAAAAGTTTAAGTTACTTTAGGGATAA | |
| | | CAGCGTTAT | |
| AY351 | Munida | AAGGGACGATAAGACCCTATAAATCTTAATATTAAGTTAT | 652 |
| 185 | tyche | TTTTAGTTAATTAATATCTGTAAATACAATAATAAAATAT | |
| | | TAAATATTATGTTGGGGTGACAAGAATATAATAAATAACT | |
| | | GTTCTAAAATTTAAACAAAATTATTTGTAAAAAAAAAAGA | |
| | | TCCTTTTTAAAGATTAAAAGTTTAAGTTACTTTAGGGATA | |
| | | ACAGCGTTAT | |
| MK847 | Munida | AAGGGACGATAAGACCCTATAAATCTTTATATTAAATTAT | 653 |
| 953 | philippinensis | TTTTATTTTATTAATACCGTAGATATAATGATAAAGTAAT | |
| | | AAATATTATGTTGGGGTGATAAGAATATAAAAAATTAACT | |
| | | GTTCTAAGATTAAAACAAAATTATTTGAAAAAAAAATAAT | |
| | | AGATCCTTTATAAAGATTAAAAGTTTAAGTTACTTTAGGG | |
| | | ATAACAGCGTTAT | |
| AY351 | Munida | AAGGGACGATAAGACCCTATAAATCTTTATATTAAATTAT | 654 |
| 107 | armilla | TTTCAATTAATTAATATTGTAAATAAAATAATAAAATAAT | |
| | | AAATATTATATTGGGGCGATGAGAATATAACAAAATAACT | |
| | | GTTCTACAATTAATACAAAATTATTTGTAATAATAATAGA | |
| | | TCCTTTATAAAGATTAAAAGTTTAAGTTACTTTAGGGATA | |
| | | ACAGCGTTAT | |
| KY230 | Munida | AAGGGACGATAAGACCCTATAAATCTTAATATTAAATTAT | 655 |
| 470 | mesembria | TTTTAGTTAATTAATAATTGTAAATATAATAATAAAATAT | |
| | | TAAATATTATGTTGGGGTGACAAGAATATAATAAATAACT | |
| | | GTTCTAAAATTAAAACAAAATTATTTGTAAAAAAAATGAT | |
| | | CCTTTATAAAGATTAAAAGTTCAAGTTACTTTAGGGATAA | |
| | | CAGCGTTAT | |
| AY351 | Munida | AAGGGACGATAAGACCCTATAAATCTTTATATTAAATTAT | 656 |
| 166 | spilota | TTTTAATTAATTAATATTGTAAATTTAATAATAAAATATT | |
| | | AAATATTATGTTGGGGCGACAAGAATATAATAAATAACTG | |
| | | TCTAAATTTAAAACAAAATTATTTGTAATAAAATAGATCC | |
| | | TTTTTAAAGATTAAAAGTTTAAGTTACTTTAGGGATAACA | |
| | | GCGTTAT | |
| KY230 | Munida | AAGGGACGATAAGACCCTATAAATCTTTATATTAGATTAT | 657 |
| 468 | benguela | TTTTAGTTATTTAATATTGTAAATATAGTAATAAAATGAT | |
| | | ATATATTGTGTTGGGGTGATGAGAATATAATAAATAACTG | |
| | | TTCTAAAATGAAAACAAAATTATTTGAATAAATAGTAGAT | |
| | | CCTTTATTGAGATTAAAAGTTTAAGTTACTTTAGGGATAA | |
| | | CAGCGTTAT | |
| MK847 | Munida | AAGGGACGATAAGACCCTATAAATCTTTATATTAAATTAT | 658 |
| 934 | endeavourae | TTTTAATTAGTTAATATTGTAAATATAATAGTAAGGTAAA | |
| | | GTATATTGTGTTGGGGTGATGAGAATATAAGAAATAACTG | |
| | | TTCTAAAATAAAAACAAAATTATTTGAAGAAATAATAGAT | |
| | | CCTTTGTTGAGATTAAAAGTTTAAGTTACTTTAGGGATAA | |
| | | CAGCGTTA | |
| MK847 | Munida | AAGGGACGATAAGACCCTATAAATCTTAATATTAAATTAT | 659 |
| 961 | agave | TTTTAGTTAATTAATAGTTGTAAATATAATAATAAAATAT | |
| | | TAAATATTATGTTGGGGTGACAAGAATATAATAAATAACT | |
| | | GTTCTAAAATTTAAACAAAATTATTTGTAAAAAAAGATCC | |
| | | TTTATAAAGATTAAAAGTTTAAGTTACTTTAGGGATAACA | |
| | | GCGTTAT | |
| MK458 | Munida | GAGGGACGATAAGACCCTATAAATCTTTATATTAGGTTAT | 660 |
| 566 | idyia | TTTCAGTTGATTAATATTGTAAATATAATAATAAGATAAT | |
| | | ATATATTGTGTTGGGGTGATGAGAATATAATGAAATAACT | |
| | | GTTCTAGGATAGAAACAAAATTATTTGAAGTAATAAATGA | |
| | | TCCTTTATTGAGATTAAAAGTTTAAGTTACTTTAGGGATA | |
| | | ACAGCGTTAT | |
| AY351 | Munida | AAGGGACGATAAGACCCTATAAATCTTTATATTAGATTAT | 661 |
| 143 | militaris | TTTTAGTTATTTAATATTGTAAATATAATAATAAAGTGAT | |
| | | TTATATTGTGTTGGGGTGATGAGAATATAATAAATAACTG | |
| | | TTCTAAAATGAAAACAAAATTATTTGTGTAAATAATAGAT | |
| | | CCTTTATTGAGATTAAGAGTTTAAGTTACTTTAGGGATAA | |
| | | CAGCGTTAT | |
| MF490 | Munida | AAGGGACGATAAGACCCTATAAATCTTTATATTAACTTAT | 662 |
| 158 | flinti | TTTTTATATATTAATCTTGTAAACTCCGTTTAATAAAATA | |
| | | AGATATATTTTGTTGGGGCGACAAGAATATAAACACAACT | |
| | | GTTCTAAATACTAAACAAAACTATTTGTTTTAAATTCATA | |
| | | GATCCTTCATTTAAGATTATAAGTTTAAGTTACTTTAGGG | |
| | | ATAACAGCGTTAT | |
| AY351 | Munida | AAGGGACGATAAGACCCTATAAATCTTTATATTAGATTAT | 663 |
| 115 | congesta | TTTTAGTTATTTAATATTGTAAATATAGTAATAAAATAAT | |
| | | ATATATTGTGTTGGGGTGATGAGAATATAATAAATAACTG | |
| | | TTCTAAAATATAAACAAAATTATTTGAATAAATAGTAGAT | |
| | | CCTTTATTGAGATTAAAAGTTTAAGTTACTTTAGGGATAA | |
| | | CAGCGTTAT | |
| AY351 | Munida | AAGGGACGATAAGACCCTATAAATCTTTATATTAGATTAT | 664 |
| 163 | rubridigitalis | TTTTAAGTTGTTTAATATTGTAAATATAATAATAAAATAA | |
| | | TATATATTGTGTTGGGGTGATGAGAATATAATAAATAACT | |
| | | GTTCTAAAATGAAGACAAAATTATTTGTATAAATAATAGA | |
| | | TCCTTTATTGAGATTAAAAGTTTAAGTTACTTTAGGGATA | |
| | | ACAGCGTTAT | |
| KF182 | Munida | AAGGGACGATAAGACCCTATAAATCTTTATATTAAATTAT | 665 |
| 521 | iris | TTTTAATTTGTTAATTATGTAAATTTAATAACAAGATAAA | |
| | | ATATATTATGTTGGGGTGATGAGAATATATAATCAACTGT | |
| | | TCTAAATTAAAAACAAAATTATTTGAACAAAATAGTAGAT | |
| | | CCTTATTAGAGATTAAAAGTTTAAGTTACTTTAGGGATAA | |
| | | CAGCGTTAT | |
| MF490 | Munida | AAGGGACGATAAGACCCTATAAATCTTTATATTAAATTAT | 666 |
| 159 | microphth | TTTAAATTAATTAATATTGTAAATAAGATAATAAAATAAT | |
| | alma | ATATATTGTGTTGGGGTGATGAGAATATAATAAATAACTG | |
| | | TTCTAAAATAAAAACAAAGCTATTTGTATAAAATAATAGA | |
| | | TCCTTTACTAAGATTAAAAGTTTAAGTTACTTTAGGGATA | |
| | | ACAGCGTTAT | |
| AY351 | Munida | GGGGGACGATAAGACCCTATAAATCTTTATATTAAGTTAT | 667 |
| 164 | rufiantenn | TTTTAGTTAATTAATATTGTAAATATAATAATAAAATAGC | |
| | ulata | GTGTATTGTGTTGGGGTGATGAGAATATAATAAATAACTG | |
| | | TTCTAAAATGTAAACAATATTATTTGTAAATAATAGTAGA | |
| | | TCCTTTATTGAGATTAAAAGTTTAAGTTACTTTAGGGATA | |
| | | ACAGCGTTAT | |
| KF182 | Munidapusilla | AAGGGACGATAAGACCCTATAAATCTTTATATAAAATTAT | 668 |
| 522 | | CTTTAATTTATTTATAATGTAAGTTTAATATAAAGATAAG | |
| | | ATATATTACATTGGGGTGATGAGAATATATAATAAACTGT | |
| | | TCTAGATTCAAAACAAATTTATTTGTATTATAAAATAGTA | |
| | | GATCCTTTTTTAAGATTAAAAGTTTAAGTTACTTTAGGGA | |
| | | TAACAGCGTTAT | |
| KY230 | Munida | AAGGGACGATAAGACCCTATAAATCTTTATATTAGATTAT | 669 |
| 472 | remota | TTTTAGTTATTTAATATTGTGAATATAATAATAAAGTGGT | |
| | | ATATATTGTGTTGGGGTGATGAGAATATAATAAATAACTG | |
| | | TTCTAAAATTAAAACAAAATTATTTGTATAAATAATAGAT | |
| | | CCTTTATTGAGATTAAAAGTTTAAGTTACTTTAGGGATAA | |
| | | CAGCGTTAT | |
| AY351 | Munida | AAGGGACGATAAGACCCTATAAATCTTTATATTAATTTAT | 670 |
| 141 | leptosyne | TTGTTTATTGATTAATAAGTAGATGATATAATAAGATAAT | |
| | | ATATATTATGTTGGGGTGATGAGAATATAATAATTAACTG | |
| | | TTCTAAGTTGTAAACAAAATTATTTGAAATTTTATAGATC | |
| | | CTTTATTGAGATTAAAAGTTTAAGTTACTTTAGGGATAAC | |
| | | AGCGTTAT | |
| AY351 | Munida | AAGGGACGATAAGACCCTATAAATCTTTATATTAGATTAT | 671 |
| 162 | rosula | TTTTAGTTATTTAATATTGTAAATATAATAATAAAGTGAT | |
| | | ATATATTGTGTTGGGGTGATGAGAATATAATAAATAACTG | |
| | | TTCTAAAATGAAAACAAAATTATTTGTGTAAATAATAGAT | |
| | | CCTTTATTGAGATTAAAAGTTTAAGTTACTTTAGGGATAA | |
| | | CAGCGTTAT | |
| LC464 | Munida | TCTTTATATTAAACTATTTAAAATTAATTAATATGTAAAT | 672 |
| 553 | munin | TTAATAAATAAAATGGAAAATATTATATTGGGGTGATAAG | |
| | | AATATAAATTATATAACTGTTCTAAAATTTAAACAAAATT | |
| | | ATTTGTACAAATAGTAGATCCTTTTTTAAGATTAAAAATT | |
| | | TAAGTT | |
| JN800 | Munida | AAGGGACGATAAGACCCTATAAATCTTTATATTAAGTTAT | 673 |
| 548 | valida | TTTAAATTAATTAATGTTGTAAATAAAATAATAAAGTAAT | |
| | | ATATATTGTGTTGGGGTGATGAGAATATAATAAATAACTG | |
| | | TTACTAAATTTAAAAACAAGTTATTTGAATTTAAAATAAT | |
| | | AGATTCCTTTTTGAGTATTAAAAGTTTAAGTTACTTTAGG | |
| | | GATAACAGTCGTTA | |
| AY351 | Munida | GAGGGACGATAAGNCCCTATAAATCTTAATATTAAATTAT | 674 |
| 153 | proto | TTTTAATTAATTAATATTGTAAATATAATAATAAAATATT | |
| | | AAATATTATGTTGGGGTGATAAGAATATAAAAAATAACTG | |
| | | TTCTAAATTTAAAACAAAATTATTTGAAAATAATAGATCC | |
| | | TTTTTTAAAGATTAAAAGTTCAAGTTACTTTAGGGATAAC | |
| | | AGCGTTAT | |
| AY351 | Enriquea | AAGGGACGATAAGACCCTATAAAGCTTTATATTATTTTTT | 675 |
| 142 | leviantennata | TTTTATAGATTATTGTTGTGGATATAATAATAAAATAAGG | |
| | | AATATTATATTGGGGTGATAAGGGTATAAAATTAACTGCT | |
| | | CTAAAATTAAATCAAATTTATTTGTAAAATAATAATAGAT | |
| | | CCTTTATTAAGATTAAAAGTTTAAGTTACTTTAGGGATAA | |
| | | CAGCGTTAT | |
| LC430 | Munida | TCTTAATATTAAACTATTTTTAGTTAATTAATTAATTGTA | 676 |
| 735 | multilineata | AATATAAATAATAAAATATTAAATATTATGTTGGGGTGAC | |
| | | AAGAATATAAAAAATAACTGTTCTAAATTTAAAACAAAAT | |
| | | TATTTGAAAAAAAGATCCTTTATAAAGATTAAAAGTTCAA | |
| | | GTT | |
| AY351 | Munida | AAGGGACGATAAGACCCTATAAATCTTTATATTATATTAT | 677 |
| 152 | pagesi | TTTTAGTTAATTAATATTGTAAATATAATAATAAGGTAAT | |
| | | ATATATTATGTTGGGGTGATAAGAATATAATAAATAATTG | |
| | | TTCTAAAATGAAAACAAAATTGTTTGTGTTAATAATAGAT | |
| | | CCTTTATTGAGATTAAAAGTTTAAGTTACTTTAGGGATAA | |
| | | CAGCGTTAT | |
| KY230 | Munida | GAGGGACGATAAGACCCTATAAATCTTTATATTAAGTTGT | 678 |
| 475 | stomifera | TTTTAGCTGATTAATGTTGTAGATTCAGTATTAAGACAAA | |
| | | ATATATTATGTTGGGGTGATGAGAATACAATAGATACCTG | |
| | | TTCTAAAATATCAAACAAAAATTTTTGTGTGAGTAGAAGA | |
| | | TCCTCTTTATTGAGATCAGGAGTTTAAGTTACTTTAGGGA | |
| | | TAACAGCGTTAT | |
| AF436 | Munida | GGGACGATAAGACCCTATAAATCTTTATATTAAATTGTTT | 679 |
| 050 | quadrispina | TTAGTTTATTAATAATGTAATTTAGATAATAAGGCAAAAT | |
| | | ATATTGTGTTGGGGTGATGGGAATATAAAAAATTAACTGT | |
| | | TCTGAAATAAAAACAAATTTGTTTGGCTTAATAATAGATC | |
| | | CTTTTTTAAGATTAAAAGTTTAAGTTACTTTAGGGATAAC | |
| | | AGCGTTAT | |
| AY351 | Munida | AAGGGACGATAAGACCCTATAAATCTTTATATTAAGTTGT | 680 |
| 183 | tiresias | TTTTAGTTAATTTATATTGTAAATAAGATAATAAAGTAAA | |
| | | ATATATTGTGTTGGGGTGATGAGAATATAATAAATAACTG | |
| | | TTCTAAAATAAAAACAAAGCTATTTGTATTAAATAATAGA | |
| | | TCCTTTATTAAGATTAAAAGTTTAAGTTACTTTAGGGATA | |
| | | ACAGCGTTAT | |
| AY351 | Munida | ATGGGACGATAAGACCCTATAAATCTTTATATTAAGTTAT | 681 |
| 159 | psylla | TTTTAGTTAATTAATATTGTAAATATAATAATAAAATAAT | |
| | | GTATATTGTGTTGGGGTGATGAGAATATAATAAATAACTG | |
| | | TTCTAAAATGTAAACAATATTGTTTGTAATAATAGTAGAT | |
| | | CCTTTATTGAGATTAAAAGTTTAAGTTACTTTAGGGATAA | |
| | | CAGCGTTAT | |
| MK847 | Munida | AAGGGACGATAAGACCCTATAAATCTTTATATTAAACTGT | 682 |
| 965 | heteracantha | TTTCAATTAATTAATATTGTAAATATAATAATAAAATAGT | |
| | | AAATATTATGTTGGGGTGATGAGAATATAAGAAATAACTG | |
| | | TTCTAAAATTAAAACAAAATTGTTTGTAATAATAATAGAT | |
| | | CCTTTATAAAGATTAAAAGTTTAAGTTACTTTAGGGATAA | |
| | | CAGCGTTAT | |
| FJ462 | Paralomis | AAAAGACGATAAGACCCTATAAATCTTTACAAGAAATATA | 683 |
| 645 | formosa | TTTTATGTTTTAGCTTATAAGTATATAAGAAATAAAAATA | |
| | | TAAGTTTGTTGCGCTGGGGCGGCGTAGATATATAAATAAA | |
| | | CTGTCTATGGTTTAAATACAATAATTATTGCTTAATACAA | |
| | | ATTGATCCTTAAATAGATTAAAAGATTAAGATACTTTAGG | |
| | | GATAACAGCGTTAT | |
| FJ462 | Paralomis | AAAAGACGATAAGACCCTATAAATCTTTACAAGAAATATA | 684 |
| 646 | spinosissima | TTTTATGTTTTAGCTTATAAGTGTATAAGAAATAAAAATA | |
| | | TAAGTTTGTTGCGCTGGGGCGGCGTAGATATATAAATAAA | |
| | | CTGTCTATGATTTAAATACAATAATTATTGYTTAATACAR | |
| | | ATTGATCCTTAAATAGATTAAAAGATTAAGATACTTTAGG | |
| | | GATAACAGCGTTAT | |
| KY426 | Paralomis | AAAAGACGATAAGACCCTATAAATCTTTACAAGAAATATA | 685 |
| 326 | birsteini | TTTTATGTTTTAGCTTATAAGTGTATAAGAAATAAAAATA | |
| | | TAAGTTTGTTGCGCTGGGGCGGCGTAGATATATAAATAAA | |
| | | CTGTCTATGGTTTAAATACAATAATTATTGCTTAATACAG | |
| | | ATTGATCCTTAAATAGATTAAAAGATTAAGATACTTTAGG | |
| | | GATAACAGCGTTAT | |
| KY426 | Paralomis | AAAAGACGATAAGACCCTATAAATCTTTACAAGAAATATA | 686 |
| 327 | hirtella | TTTTATGTTTTAGCTTATAAGTGTATAAGAGATAAAAATA | |
| | | TAAGTTTGTTGCGCTGGGGGGGCGTAGATATATAAATAAA | |
| | | CTGTCTGTGGTTTAAATACAATAATTATTGCTTAATACAA | |
| | | ATTGATCCTTAAATAGATTAAAAGATTAAGATACTTTAGG | |
| | | GATAACAGCGTTAT | |
| MF460 | Scyllarus | AAGGGACGATAAGACCCTATAAATCTTTATACTTGATTTT | 687 |
| 387 | subarctus | ATAGAGCAAAATATTAAAATAATTTTTGTTCAATAGGTTT | |
| | | GGGTATTTTGTTGGGGCGACAGGAGTATAAAATGTAACTG | |
| | | CTCTAAAATGATAGTTAGTTGATATTTAGTTAAGTGATCC | |
| | | TTTAATAAAGATGTTAGTTTAAGTTACTTTAGGGATAACA | |
| | | GCGTAAT | |
| FJ174 | Scyllarus | GGGGGACGATAAGACCCTATAAATCTTTATATTTAAGCTT | 688 |
| 908 | pygmaeus | ATAAAATAAACGATTAAAATAATTTTTGTTTAATAAGCTT | |
| | | ATATATTTTGTTGGGGCGCCAGAAGTATAAAATGTAACTG | |
| | | CTTTAAAATAATAGTTAAATGATATTTAGTTAAGTAATCC | |
| | | TTTAATAAAGATGTTAGTTTAAGTTACTTTAGGGATAACA | |
| | | GCGTAAT | |
| JN701 | Scyllarus | GGGGGACGATAAGACCCTATGAATCTTTATATTTCGGTTT | 689 |
| 734 | chacei | ATAAGATAAAAAATTAATATAATTTTTGTCTTATTAATCT | |
| | | TTTTATTTTGTTGGGGCGACAGAAGCGTAAACTGTAACCG | |
| | | CTTTGAAAAAATAGTTAATGATATTTAGTTAAGTGACCCT | |
| | | TCTATGGGGGTATCAGTATAAGTTACTCTAGGGATAACAG | |
| | | CGTAAT | |
| FJ174 | Scyllarus | AGGGGACGATAAGACCCTATAAATCTTTATATTTGGGTTT | 690 |
| 909 | caparti | GTGAAATAAAAAATTAAAATAATTTTTGTTTAATAAGCTT | |
| | | GGGTATTTTGTTGGGGCGACAAGAGTATAAAAATGTAACT | |
| | | GCTTTGAAATAATAGTTAATTGGTGTTTAGTTAATTGATC | |
| | | CTTTAGTAGAGATGTCAGTTTAAGTTACTTTAGGGATAAC | |
| | | AGCGTAAT | |
| JN701 | Scyllarus | GGGGGACGATAAGACCCTATGAATCTTTATTTTTGGCTTA | 691 |
| 732 | americanus | TAAAATAAAAAAATAATATCAATTTTTGTTTTATCGGTTT | |
| | | CAATATTTTGTTGGGGCGACGGAAGCGTAAATTGTAACCG | |
| | | CTTTATATTAATAGTTAACGATATTTAGTTAAGTGATCCT | |
| | | TTATAGAGATATTAGTATAAGTTACTCTAGGGATAACAGC | |
| | | GTAAT | |
| FN295 | Episesarma | AAAAGACGATAAGACCCTATAAAACTTAATAAAAATTGAT | 692 |
| 576 | palawanense | ATTTAAATAAAATTTATTAATAAATATTTAGTAATTAATT | |
| | | TTTATTTTATTGGGGTGATAATGGTAAAATGGATATTAAC | |
| | | TGTTAATAAAGTTTATAACAAAAATTATTGAGTAAGTACA | |
| | | ATAAATGATCCTTATTAAAGATTAAAAGTTTAAGTTACTT | |
| | | TAGGGATAACAGCGTTAT | |
| FN295 | Episesar | AAAAGACGATAAGACCCTATAAAACTTAATATAAATTGAT | 693 |
| 575 | masingaporense | TATTTGAGTAAAATTTTATTGTATAAATATTTAATAATTA | |
| | | ATTTATATTTTATTGGGGTGATAATGGTAAAATGATTATT | |
| | | AACTGTTAATTATTTTATGACAAAAATTATTGAATAAATG | |
| | | TGTAATAAATGATCCTTATTAAAGATTAAAAGTTTAAGTT | |
| | | ACTTTAGGGATAACAGCGTTAT | |
| KP712 | Austropotamobius | GGGGGACGATAAGACCCTATAAAACTTTATATTTTAAAAA | 694 |
| 873 | fulcisianus | TATTAATTAATTTTGTATGAGAAGGCTATTTTTAAATATT | |
| | orientalis | TTATTGGGGTGATAGGGATATAATAAAAGATAACTGTTTC | |
| | | TTTTTTTTACAGAGGTGTTTGAGTAAAAGATCCTAATTAG | |
| | | GGAAAGAAGGTTAAGTTACTTTAGGGATAACAGCGTAAT | |
| FJ152 | Achelous | GGGGGACGATAAGACCCTATAAAGCTTCATAAATTTATAG | 695 |
| 150 | tumidulus | ATTTTATGCATTTAATATAAAAATAGTTTCTGCAGTATTT | |
| | | ATTTGGTTGGGGCGACAAAAGTATAATTTTTAATAACTGC | |
| | | TATTTGTAAGTTACAATTATATTTGGTTAGTGAGTAATTG | |
| | | ATCCTTCTGTGAGGAGTAAAAGTTCAAGTTACTTTAGGGA | |
| | | TAACAGCGTCAT | |
| MG51 | Achelous | GGGGGACGATAAGACCCTATAAAGCTTTATAAATTCAAGA | 696 |
| 5565 | asper | ATTTTATATATTTAGTATAAAAATAATTTTCTCGATGTTT | |
| | | ATTTGGTTGGGGCGACAAAAGTATAATTTTGAATAACTGC | |
| | | TAGTTCAGACATACAAAAATCTCTGATTTATAAATAATGG | |
| | | ATCCCTTTATGAGGATTAAAAGTTCAAGTTACTTTAGGGA | |
| | | TAACAGCGTTAT | |
| DQ388 | Achelous | GAGGGACGATAAGACCCTATAAAGCTTTATAAGTTTATAA | 697 |
| 067 | sebae | ATTTTATACATTTGTAAATAAAAATAATTTTTTTAATACT | |
| | | TATTTGGTTGGGGCGACAAAAGTGTAATTTAAAATAACCG | |
| | | CTAATATATTAATACAATAATTTTTGGCTAACTAAGTAAT | |
| | | GGATCCCTTTTAAGGAATAAAAGACCAAGTTACTTTAGGG | |
| | | ATAACAGCGTTAT | |
| MG51 | Portunus | GGAAGACGATAAGACCCTATAAAGCTTTATAAGTTTATAA | 698 |
| 5559 | acuminatus | ATTTTGTATATTTAACCTAAAAATAATTTTTTAGGTGCTT | |
| | | ATTTGGTTGGGGCGACAAAAGTATAATTACATTGATTTTT | |
| | | TATTTAAAAATAATGGATCCTTCTGTGAAGAATAAAAGTT | |
| | | CAAGTTACTTTAGGGATAACAGCGTAAT | |
| MG51 | Achelous | GGGGGACGATAAGACCCTATAAAGCTTTATAAGTTTATGG | 699 |
| 5578 | tuberculatus | ATTTTATAGATTTARAATAAAGATAATTTCCTGAATATTT | |
| | | ATTTGGTTGGGGCGACAAAAGTATAATTTAAATAACTGCT | |
| | | AGTTGAAGATTACAGTGATTTTTGATTCTAAAATAATGGA | |
| | | TCCCTTATAAGGATTAAAAAATCAAGTTACTTTAGGGATA | |
| | | ACAGCGTTAT | |
| MG51 | Achelous | GGGGGACGATAAGACCCTATAAAGCTTTATAAATTTATGA | 700 |
| 5567 | iridescens | ATTTTATTTATTTAATATAAAAATAATTTTTACAATATTT | |
| | | ATTAGGTTGGGGCGACAAAAGTATAATTCGAAATAACTGC | |
| | | TATTTTAAAATTACAATAGTTTTTGTTTTAAAAGTAATTG | |
| | | ATCCCTTTATGAGGAGTAAAAGTTCAAGTTACTTTAGGGA | |
| | | TAACAGCGTTAT | |
| MG51 | Portunus | GGGGGACGATAAGACCCTATAAAGCTTCATAAATTTATAA | 701 |
| 5580 | xantusii | ATTTTATGTATYTAATATAAAGATAATTTTTGTAACGTTT | |
| | | ATTTGGTTGGGGCGACAAAAGTATAATTTCAAATAACTGC | |
| | | TAGGTTTGATTTACAATGATAGTTGATTAATAAGTAATTG | |
| | | ATCCCTTGTGAGGAATAAAAGTTCAAGTTACTTTAGGGAT | |
| | | AACAGCGTTAT | |
| DQ388 | Achelous | GGGGGACGATAAGACCCTATAAAGCTTTATAAATTTATTA | 702 |
| 065 | depressifrons | ATTTTGTATATTTAAAATAAAAACAATCTTTTCAATATTT | |
| | | ATTTGGTTGGGGCGACAAAAGTGTAATTAAAGTAACCGCT | |
| | | AATAATATAATTACAATAATTTTTGATTAGCAATTAATGG | |
| | | ATCCCTTTTAAGGAATAAAAGATCAAGTTACTTTAGGGAT | |
| | | AACAGCGTTAT | |
| DQ388 | Achelous | AGGGGACGATAAGACCCTATAAAGCTTTATAAGTTTATGG | 703 |
| 063 | rufiremus | ATTTTATATATTTAATATAAAAATAATTTTTTAAATATTT | |
| | | ATTTGGTTGGGGCGACAAAAGTATAATTGGAAATAACTGC | |
| | | TAACTTTAAGTACCACTACATCTTGTATAAAAAATAATCG | |
| | | GATTCTTTATAAAGAGTAAAAGTTCAAGTTACTTTAGGGA | |
| | | TAAAAGCATCAT | |
| DQ388 | Achelous | GGGGGACGATAAGACCCTATAAAGCTTTATAAATTTAAAA | 704 |
| 057 | gibbesii | ATTTTATATATTTATCATAAAGATAATTTTTCAATGTTTA | |
| | | TTTGGTTGGGGCGACAAAAGTATAATTTAAAATAACTGCT | |
| | | AGTTTGAAAACACAATAATTTTTGTTTTAAAAAGTAATGG | |
| | | ATCCCTTTATGAGGATTAAAAGTTCAAGTTACTTTAGGGA | |
| | | TAACAGCGTCAT | |
| MG51 | Portunus | GGGGGACGATAAGACCCTATAAAGCTTTATAAATTTATAA | 705 |
| 5573 | minimus | ATTTTATGTATTTAATATAAAGATAATTTTTGTAACGTTT | |
| | | ATTTGGTTGGGGCGACAAAAGTATAATTTCAAATAACTGC | |
| | | TAGGTTTGGTTTACAATGATAGTTGATTAATAAGTAATTG | |
| | | ATCCCTTGTGAGGAATAAAAGTTCAAGTTACTTTAGGGAT | |
| | | AACAGCGTTAT | |
| MG51 | Achelous | GGAAGACGATAAGACCCTATAAAGCTTTATAAGTCTACGA | 706 |
| 5576 | stanfordi | ATTCTGTAGATCTGATATAAAAATAATTTTTTTGATACTT | |
| | | ATTTGGTTGGGGCGACAAAAGTATAATTTAAATAACTGCT | |
| | | AATTTAAAATTACAATGATTTTTGATTATAAAAATAATGG | |
| | | ATCTTTCTCTGAAAAATGAAAGTTCAAGTTACTTTAGGGA | |
| | | TAACAGCGTAAT | |
| MG51 | Achelous | GGGGGACGATAAGACCCTATAAAGCTTTATAAATTTATAA | 707 |
| 5566 | brevimanus | ATTTTATGTATTTAATATAAAGATAATTTTTGTAACGTTT | |
| | | ATTTGGTTGGGGCGACAAAAGTATAATTTTAAATAACTGC | |
| | | TAGGTTTGATTTACAGCGATAGTTGATTAATAAGTAATTG | |
| | | ATCCCTTGTAAGGAATAAAAGTTCAAGTTACTTTAGGGAT | |
| | | AACAGCGTTAT | |
| MG51 | Portunus | GGGGGACGATAAGACCCTATAAAGCTTCATAAATTTATAA | 708 |
| 5560 | affinis | ATTTTATGTATCTAATATAAAGATAATTTTTGTAACGTTT | |
| | | ATTTGGTTGGGGCGACAAAAGTATAATTTCAAATAACTGC | |
| | | TAGGTTTGATTTACAATGATAGTTGATTAATAAGTAACTG | |
| | | ATCCCTTGTGAGGAATAAAAGTTCAAGTTACTTTAGGGAT | |
| | | AACAGCGTTAT | |
| MG51 | Achelous | GGGGGACGATAAGACCCTATAAAGCTTTATAAATTTATAG | 709 |
| 5561 | angustus | ATTTTATTTATTTAATAATAAAAATAATTTCTACAGTGTT | |
| | | TATTTGGTTGGGGCGACAAAAGTATAATTTTGAATAACTG | |
| | | CTAGTTCTAAGTTACAACAATATTTGATTTGTAAGTAATT | |
| | | GATCCTTCTATGAGGAGTAAAAGTTCAAGTTACTTTAGGG | |
| | | ATAACAGCGTTAT | |
| DQ388 | Achelous | GGGGGACGATAAGACCCTATAAAGCTTTATAAATTTTATA | 710 |
| 062 | binoculus | GACTTTATATATTTAAAATAAAAATAATTTCTAAAACATT | |
| | | TATTTGGTTGGGGCGACAAAAGTATAATTTCAAATAACTG | |
| | | CTATTTTAAAATTACAATAATTTTTGTTTTATGAATAATT | |
| | | GATCCTTCTATGAGGAGTAAAAGTTCAAGTTACTTTAGGG | |
| | | ATAACAGCGTCAT | |
| MH168 | Oratosquillina | GAGGGACGATAAGACCCTATAAAGCTTTATATTACATTTC | 711 |
| 220 | inornata | TTTTTTATAAATTATTTCGTTATTAACTTATTTTAGAATT | |
| | | TGTATTATTTTACTGGGGGGGTAAAAGTATATTAAAGATT | |
| | | AACTGCTTTTGTTTTGTAAAACAATTATAGTTGAAAATAA | |
| | | ATTGATCCATTATTAATGATCATAAGAACAAGTTACTTTA | |
| | | GGGATAACAGCGTAAT | |
| MH168 | Oratosquillina | GAGGGACGATAAGACCCTATAAAGCTTTATGTTATTCTTC | 712 |
| 238 | asiatica | TTTTTTATAAATTATTTAATTATTAACTTATTTCAGAATT | |
| | | AATATCATTTTACTGGGGCGGTAAAAGTATAATAAAGATT | |
| | | AACTGCTTTTGTTTTGTCTAACAATAATAGTTGAAAATAG | |
| | | ATTGATCCATTATTAATGATCATAAGAACAAGTTACTTTA | |
| | | GGGATAACAGCGTAAT | |
| MH168 | Oratosquillina | GAGGGACGATAAGACCCTATAAAGCTTTATATTACATTTC | 713 |
| 217 | anomala | TTTTTTATAAATTATTTCGTTATTAACTTATTTTAGAATT | |
| | | TGTATTATTTTACTGGGGCGGTAAAAGTATAATGAAGATT | |
| | | AACTGCTTTTGTTTTGTAAAACAATTATAGTTGAAAATAA | |
| | | TTTGATCCATTATTAATGATCATAAGAACAAGTTACTTTA | |
| | | GGGATAACAGCGTAAT | |
| MH168 | Oratosquillina | GGAGGGACGATAAGACCCTATAAAGCTTTATGTTACATTT | 714 |
| 218 | perpensa | CTTTTTTATAAATTATTTTGTTATTAACTTATTTTAGAAT | |
| | | TTGTATCATTTTACTGGGGGGGTAAAAGTATAATAAAGAT | |
| | | TAACTGCTTTTGTTTTGTATAACAATTTTAGTTGGAAATA | |
| | | AATTGATCCATTATTAATGATCATAAGAACAAGTTACTTT | |
| | | AGGGATAACAGCGTAAT | |
| MH168 | Erugosquilla | GAGGGACGATAAGACCCTATAAAGCTTTATGATATTCTTC | 715 |
| 226 | graham | TTTTTTATAAATTGTCTTGTTATTAACTTATTTTAGAATT | |
| | | AATATCATTTTACTGGGGCGGTAAAAGTATAAAAAGATTA | |
| | | ACTGCTTTTGTTTTGTATAACAATCTTAGTTGGAAATAAA | |
| | | TTGATCCATTATTAATGATCATAAGAACAAGTTACTTTAG | |
| | | GGATAACAGCGTAAT | |
| MH168 | Busquilla | GAGGGACGATAAGACCCTATAAAGCTTTATGATATTTCTC | 716 |
| 223 | quadraticauda | TTTCTTATAAATTTTTGTTATTAACCTATTTTAGAGTTAA | |
| | | TATCATTTTACTGGGGCGGTAAAAGTATAATAAAGATTAA | |
| | | CTGCTTTTGTTTTGTACAACAATTTTAGTTGGAAATAAAT | |
| | | TGATCCATTATTAATGATCATAAGACCAAGTTACTTTAGG | |
| | | GATAACAGCGTAAT | |
| MH168 | Kempella | GAGGGACGATAAGACCCTATAAAGCTTTATAATACCTTTC | 717 |
| 213 | stridulans | TTTTTTATAAAATTTTTTGTTGTTAACATATTTTAGAATT | |
| | | TGTATTATTTTACTGGGGCGGTAAAAGTATAATAAATAAC | |
| | | TGCTTTTATTTTGTGTAACAATTTTAGTTGATGATAAATT | |
| | | GATCCATTATTAATGATTATAAGAACAAGTTACTTTAGGG | |
| | | ATAACAGCGTAAT | |
| AF133 | Gonodactylaceus | GAGGGACGATAAGACCCTATAAAGCTTTATAGTAGTTCCT | 718 |
| 678 | graphurus | TTTTATTCAAATTATAAAATGTTAACCTTTTGAAAGATTA | |
| | | CTACTATTTTACTGGGGCGGTAGAAGTATATTTATGATTA | |
| | | ACTGCTTTTGTTTTGACAAACAATAATAATTGAATATTTA | |
| | | TGTGATCCATTATTAATGATCAAAAGTACAAGTTACTTTA | |
| | | GGGATAACAGCGTAAT | |
| MW01 | Gonodactylaceus | AAGTGACGATAAGACCCTATAAAGCTTTATAGTAATTTCT | 719 |
| 9425 | randalli | TTTTATTTAAAATATAAAATTTTAACCTTTTTAAAGATTA | |
| | | TTACTATTTTACTGGGGCGGTAAAAGTATATTTTGATTAA | |
| | | CTGCTTTTGTTTTAATTAACAACGATAGTTGAATAGTCAT | |
| | | GTGATCCATTATTAATGATCAAAAGTACAAGTTACTTTAG | |
| | | GGATAACAGCGTAAT | |
| CAU74 | Carcinus | GGGGGACGATAAGACCCTATAAAACTTTATATACACAACA | 720 |
| 327 | aestuarii | ATACAGTTAAATTAAACTATAAAAACTTATTAGATAAAAT | |
| | | ATATTTAGTTGGGGCGACTGAGGTATAATTTATAGTAACT | |
| | | GATTAAGAGCTAGACAAATAATATTTGATTAATTTTAATT | |
| | | GATCCTTTTTAAAGATTAAAAGATTAAGTTACTTTAGGGA | |
| | | TAACAGCGTAAT | |
| KF220 | Menippe | GAGGGACGATAAGACCCTATAAAGCTTTATAAATTATTTA | 721 |
| 514 | rumphii | AATTTGATTGAATTAATATAATAAAATTTTGATTTGGATT | |
| | | AATTTATTTTGCTGGGGGGGCATAGGTATAAATTATATTA | |
| | | ACTGCTTAGTAGAAATACAATAATAGTTGAGTTGACATTA | |
| | | AAAGATCCTTTTTAAAGATTTAAGAACAAGTTACTTTAGG | |
| | | GATAACAGCGTTAT | |
| MK971 | Menippe | GAGGGACGATAAGACCCTATAAAGCTTTATATATTATCTG | 722 |
| 425 | nodifrons | AATTTGAGTAAATTGATTTATAAAATTTTAATTTGGAGTA | |
| | | ATTTATTTTGCTGGGGGGGCATAGGTATAAGTTATATTAA | |
| | | CTGCTTAATAAAAATACAATTATAGTTGAGTTATTAATTA | |
| | | AAAGATCCTTTTTAAAGATTTGAGAATAAGTTACTTTAGG | |
| | | GATAACAGCGTTAT | |
| HM637 | Menippe | GAGGGACGATAAGACCCTATAAAGCTTTATATGTTATTTA | 723 |
| 973 | adina | AATTTAATTGAATTGATTTATAAATTTTAAATTTAGAGTA | |
| | | ATTTATTTTGCTGGGGCGGCATAAGTATAAGTTATATTAA | |
| | | CTGCTTAATAATGATACAATAATAGTTGAATTGTTGTTAA | |
| | | ATGATCCTTTTTAAAGATTTAAGAACAAGTTACTTTAGGG | |
| | | ATAACAGCGTTAT | |
| JX514 | Procambarus | AAGGGACGATAAGACCCTATAAAACTTTATATTTGTAAAT | 724 |
| 567 | liberorum | TAATAGCTAATTTTATTTAAATATATTATTTAAAAATATT | |
| | | TTGTTGGGGTGACAAGGATAAAATTAAAAGTAACTGTCCT | |
| | | TTTTTATTACAATAATGTTTGGTTTAATGATCCTTAAATG | |
| | | GATTTAAGATTAAGTTACTTTAGGGATAACAGCGTAAT | |
| AY214 | Procambarus | AAGGGACGATAAGACCCTATAAAACTTTATATTTATAAGG | 725 |
| 438 | toltecae | TAGAAAATTAGTTTTATTTAATGATGCTACTTTGGAGTAT | |
| | | TTGGTTGGGGTGACAAGGATAAAATATAAAATAACTGTCT | |
| | | TTTTTTTTCTACAGTTATATTTGATTTAATGATCCTAAAA | |
| | | GGGATTAAAGATTAAGTTACTTTAGGGATAACAGCGTTAT | |
| EF012 | Procambarus | AAGGGACGATAAGACCCTATAAAACTTTATATTTATAAAT | 726 |
| 344 | curdi | TAATAGTTAATTTTATTTAAATATATTATTTTAAAATATT | |
| | | TTGTTGGGGTGACAAGGATAAAATTAAAGATAACTGTCCT | |
| | | TTTTTATTACAGTAATATCTGGTTAGGTGATCCTTAAAAT | |
| | | GGATTATAAGATTAAGTTACTTTAGGGATAACAGCGTAAT | |
| AY214 | Procambarus | AAGGGACGATAAGACCCTATAAAACTTTATATCTAGAGGT | 727 |
| 435 | digueti | AGTAGGATAATTTTATTTAAGGTGTATTATTTTAAGATAT | |
| | | TTGGTTGGGGTGACCAGGATAAAATGAAAGATAACTGTCT | |
| | | TATATATTACAATAATTTTTGGGTTGTTGATCCTACAAAG | |
| | | GGATTAAAAGATTAAGTTACTTTAGGCGATAACAGCGTAA | |
| | | T | |
| EF012 | Procambarus | AAGGGACGATAAGACCCTATAAAACTTTATATTTATAAGT | 728 |
| 345 | nigrocinctus | AGTAGTTAATTTTATTTGAGTATATTATTTTAAAGTATTA | |
| | | TGTTGGGGTGACAAGGATAAAATTAAAAATAACTGTCTTT | |
| | | TTTTATTACAGTAATGTTTGGTTTAATGATCCTTAAATGG | |
| | | ATTATAAGATTAAGTTACTTTAGGGATAACAGCGTAAT | |
| JF737 | Procambarus | AAGGGACGATAAGACCCTATAAAACTTTATATTCATAAAT | 729 |
| 390 | versutus | TAGTAGTTATTTTTATTTTAAAGTATTGTTTTAGAATATT | |
| | | TGGTTGGGGTGGCAAGGATAAAATATTTAATAACTGTCTT | |
| | | TTTTTTTTACAATAATGTTTGGTTTAATGATCCTAAAAGG | |
| | | GATTTAAGATTAAGTTACTTTAGGGATAACAGCGTAAT | |
| EU433 | Procambarus | AAGGGACGATAAGACCCTATAAAACTTTATATTTGCAAGG | 730 |
| 916 | gibbus | TAGTAGAAAATTTTATTTTAATTTATTATTTTAAAGTATT | |
| | | TGGTTGGGGTGACAAGGATAAAATATAAAATAACTGTCTT | |
| | | TTTTTTTTACAGTAATGTTTGGTTTAATGATCCTAAAAAG | |
| | | GATTAAAGATTAAGTTACTTTAGGGATAACAGCGTAAT | |
| EU433 | Cambarus | AAGGGACGATAAGACCCTATAAAACTTTATATTTAAGAAT | 731 |
| 911 | pecki | GATTAATGAGTTTTGTTTTAAGGTTATTATTTTGAGATAT | |
| | | TTGGTTGGGGCGACAAGAATAGAAGAAAAAGTAACTGTTT | |
| | | TTTTTTTAATAACAAAAATGTTTGGGTTAATGATCCTAAA | |
| | | ATGGATTAAAGATTAAGTTACTTTAGGGATAACAGCGTAA | |
| | | T | |
| JX514 | Procambarus | AAGGGACGATAAGACCCTATAAAACTTTATATTTATAAGA | 732 |
| 566 | geminus | TAGTAGCTAGTTTTATTTAAAAGTATTATTTTGGAGTATT | |
| | | TGGTTGGGGTGACAAGGATAAAATATAAAATAACTGTCTT | |
| | | TTTTTTTTACAGTAATGTTTGGTTTAATGATCCTAAAAAG | |
| | | GATTAAAGATTAAGTTACTTTAGGGATAACAGCGTAAT | |
| LC430 | Charybdis | ACTTTATAAATTTAATGATTTTATATATTTTATATAAAGA | 733 |
| 785 | acuta | TAATTTTTGGAAAATTTATTTGGTTGGGGCGACAATGGTA | |
| | | TAATTTAAATAACTGCTATTAAGTTTAACAAAAATATTTG | |
| | | CTTGAAATACTGATAATTGATCCTTTATAAGGATTTTAAG | |
| | | ATCAAGTT | |
| KC163 | Creaserinus | AAGGGACGATAAGACCCTATAAAACTTTATATTTAAAGGT | 734 |
| 442 | fodiens | ATAAATTATTTTTATTTATGTGTTGGTATCTAAAAGTATT | |
| | | TGGTTGGGGCGACAAGGGCAGAATATTAAGTAACTGTCTT | |
| | | TTTTTTTGACAATAATATTTGGTTTAATGATCCTAAAAAG | |
| | | GATTAAAGATTAAGTTACTTTAGGGATAACAGCGTAAT | |
| KC163 | Fallicambarus | AAGGGACGATAAGACCCTATAAAACTTTATATTTATAAAT | 735 |
| 480 | jeanae | GGTAGACAATTTTGTTTTGGGGTATTATTTTAAAATATTT | |
| | | GGTTGGGGTGACAAGGATAAAATAAAAAATAACTGTCTTT | |
| | | TTTTTTTACAGTGATATTTGGTTTAATGATCCTAGAAGTG | |
| | | GATTATAAGATTAAGTTACTTTAGGGATAACAGCGTAAT | |
| KC163 | Creaserinus | AAGGGACGATAAGACCCTATAAAACTTTATATTTAAAGGT | 736 |
| 523 | gordoni | ATAGGTTATTTTTATTTGTGGGTTATTATCTAAAAATATT | |
| | | GGGGGGGGCGACGAGGATAGAATATTAAGTAACTGTCTTT | |
| | | TTTTAAAACAGTAATATTTGATTTAATGATCCTAAAAGGG | |
| | | ATTAAAGATTAAGTTACTTTAGGGATAACAGCGTAAT | |
| KC163 | Creaserinus | GAGGGACGATAAGACCCTATAAAACTTTATATTTGAGGGT | 737 |
| 501 | caesius | GGAAATTATTTTTATTTGGAGGTTGATATCTAAAATATTT | |
| | | TGTTGGGGCGACAAGGATAAAATATTAAGTAACTGTCTTT | |
| | | TTTTTTTACAATGATATTTGGTTTAATGACCCTAAAAAGG | |
| | | ATTAAAGATTAAGTTACTTTAGGGATAACAGCGTAAT | |
| KC163 | Fallicambarus | AAGGGACGATAAGACCCTATAAAACTTTATATTTATGAAT | 738 |
| 463 | dissitus | GATAGTTAATTTTGTTTTGGAATATTATTTTAAAATATTT | |
| | | AGTTGGGGTGACAAGGATAAAATATGAAATAACTGTCTTT | |
| | | TTTTTTTTACAGTGATATTTGGTTTAATGATCCTAAAGTG | |
| | | GATTATAAGATTAAGTTACTTTAGGGATAACAGCGTAAT | |
| KC163 | Creaserinus | AAGGGACGATAAGACCCTATAAAACTTTATATTTAAGGAT | 739 |
| 518 | danielae | ATAGACTATTTTTATTTGTGAACTATTATCAAAAGTATTT | |
| | | GGTTGGGGTGACAGGGATAGAATATAAAGTAACTGTCCTT | |
| | | TTTTTTTAACAATAATATTTGACTTAGCGATCCCTGGGAA | |
| | | GGATTAAAGATTAAGTTACTTTAGGGATAACAGCGTGAT | |
| KC163 | Fallicambarus | AAGGGACGATAAGACCCTATAAAACTTTATATTTAAGGAT | 740 |
| 509 | oryktes | ATAGACTATTTTTATTTGTAAACTATTATCAAAAGTATTT | |
| | | GGTTGGGGCGACAGGGATAGAATATAAAGTAACTGTCCTT | |
| | | TTTTTTAACAATAATATTTGACTTAACGATCCCTGGGAAG | |
| | | GATTAAAGATTAAGTTACTTTAGGGATAACAGCGTGAT | |
| KC163 | Fallicambarus | AAGGGACGATAAGACCCTATAAAACTTTATATTTTAAGGT | 741 |
| 506 | byersi | ATAGATTATTTTTGTTTATAGGTTGTTATCTGAAAATATT | |
| | | TGGTTGGGGCGACAGGGATAAAATGAGAGGTAACTGTTTT | |
| | | TTTTTTATTACAACAATGTTTGATTTAATGATCCTGAAAA | |
| | | GGATTAAAAGATTAAGTTACTTTAGGGATAACAGCGTAAT | |
| KC163 | Creaserinus | AGGGGACGATAAGACCCTATAAAACTTTATATTTAAGGGT | 742 |
| 511 | burrisi | ATAAGTTGTTTTTGTTTGTAAGTTATTATCTGAGAGTATT | |
| | | TGGTTGGGGCGACAGGAATAGGATATAAAGTAACTGTTCT | |
| | | TTTTTTTGACAATAATATTTGGTTTAATGATCCTAAAAAG | |
| | | GATTGAAGATTAAGTTACTTTAGGGATAACAGCGTAAT | |
| KC163 | Creaserinus | GAGGGACGATAAGACCCTATAAAACTTTATATTTGAAGGT | 743 |
| 450 | gilpini | AGAAGTTATTTTTATTTGAAAGTTAATATCTAAAATATTT | |
| | | TGTTGGGGCGACAAGGATAAAATATTAAGCAACTGTCTTT | |
| | | TTTTTTTACAATGATATTTGGTTTAATGATCCTAAAAAGG | |
| | | ATTAAAGATTAAGTTACTTTAGGGATAACAGCGTAAT | |
| KC163 | Fallicambarus | AAGGGACGATAAGACCCTATAAAACTTTATATTTATAAAT | 744 |
| 479 | harpi | AGTAGCTAATTTTGTTTTGAAATATTATTTTAAAATATTT | |
| | | TGTTGGGGTGACAAGGATATAACAGAAAATAACTGTCTTT | |
| | | TATGTTTACAGTAATGTCTGGTTTAATGATCTTAAGAATA | |
| | | GATTATAAGATTAAGTTACTTTAGGGATAACAGCGTAAT | |
| KC163 | Fallicambarus | AAGGGACGATAAGACCCTATAAAACTTTATATTTTATGAA | 745 |
| 444 | macneesei | TAGTAGTTAATTTTATTTTATAATATTATTTAAAAGTATT | |
| | | TGGTTGGGGTGACAAGGATAAAATAAAAAATAACTGTCTT | |
| | | TTTTTTTTACAGTAATGTTTGGTTTAATGATCCTAAAATG | |
| | | GATTGAAAGATTAAGTTACTTTAGGGATAACAGCGTAAT | |
| KC163 | Fallicambarus | GAGGGACGATAAGACCCTATAAAACTTTATATTTATAAAT | 746 |
| 468 | petilicarpus | GGTAGTTAATTTTATTTTAGAATATTATTTTGGAATATTT | |
| | | GGTTGGGGTGACAAGGATAAAATATGAAATAACTGTCTTT | |
| | | TTTTTTTACAGTGATATTTGGTTTAGTGATCCTGAAATGG | |
| | | ATTATAAGATTAAGTTACTTTAGGGATAACAGCGTAAT | |
| KC163 | Fallicambarus | GAGGGACGATAAGACCCTATAAAACTTTATATTTATAAGT | 747 |
| 434 | wallsi | GGTAGTTAATTTTATTTTATAATATTATTTTGAAATATTT | |
| | | GGTTGGGGTGACGAGGATAAAATAAAAAATAACTGTCTTT | |
| | | TTTTTTAACAGTAATGTTTGGTATGATGATCCTAAAACGG | |
| | | ATTAAAGATTAAGTTACTTTAGGGATAACAGCGTAAT | |
| KC163 | Fallicambarus | AAGGGACGATAAGACCCTATAAAACTTTATATTTATAAAT | 748 |
| 486 | strawni | GGTAGTTAATTTTGTTTTAAAATGTTATTAAAAAATATTT | |
| | | TGTTGGGGTGACAAGGATAAAATAGAAAATAACTGTCCTT | |
| | | TTTTATTACAGTAATGTTTGGTTTAATGATCCTAAAAAGG | |
| | | ATTATAAGATTAAGTTACTTTAGGGATAACAGCGTAAT | |
| KC163 | Fallicambarus | AAGGGACGATAAGACCCTATAAAACTTTATATTTATAAGT | 749 |
| 474 | devastator | GGTAATTAATTTTATTTAGGAATATTATTTTAAAATATTT | |
| | | GGTTGGGGTGACAAGGATAAAAGTAAAAATAACTGTCCTT | |
| | | TTTTTTTTCAGTAATATCTGGTTTGATGATCCTAAAATGG | |
| | | ATTATAAGATTAAGTTACTTTAGGGATAACAGCGTAAT | |
| KC163 | Fallicambarus | AAGGGACGATAAGACCCTATAAAACTTTATATTTTATGAA | 750 |
| 432 | houstonensis | TGGTAGTTAATTTTATTTTGTAATATTATTTAAAAGTATT | |
| | | TGGTTGGGGTGACAAGGATAAAATAAAAAATAACTGTCTT | |
| | | TTTTTTTTACAGTAATGTTTGGTTTAATGATCCTAAGATG | |
| | | GATTGAAAGATTAAGTTACTTTAGGGATAACAGCGTAAT | |
| KC163 | Fallicambarus | AGGGGACGATAAGACCCTATAAAACTTTATATTTAAGGTG | 751 |
| 472 | hortoni | GAAATTATTTTTATTTGTTAAGTACTATTTAAAAATATTT | |
| | | GGTTGGGGCGACAAGGATAGAATGTTAAGTAACTGTCTTT | |
| | | TTTTTTAACAATAATATTTGGTTTAATGATCCTAAAAAGG | |
| | | ATTAAAGATTAAGTTACTTTAGGGATAACAGCGTAAT | |
| FM208 | Arenaeus | GAGGGACGATAAGACCCTATAAAGCTTTATAAGTTAAAGT | 752 |
| 749 | cribrarius | ATTTTGTATATTTTATATTATAATAATGATTTATTGAGTA | |
| | | CTTATTTGGTTGGGGCAACAAGAGTATAATAAAAAGTAAC | |
| | | TGCTAATGTTTAAAACAATTATATTTGATTTAAGAGTAAT | |
| | | TGATCCCCCAGGGGATTAAAAGATCAAGCTACTTTAGGGA | |
| | | TAACAGCGTAAT | |
| JX514 | Cambarus | AAGGGACGATAAGACCCTATAAAACTTTATATTGAAAAGT | 753 |
| 521 | tenebrosus | GATAGGTATATTTTGTTTAAAGAATTATTACTTTAAAGTA | |
| | | TTTGGTTGGGGTGACAAGAATAGAAGAGAAAGTAACTGTT | |
| | | TTTTTTTTATACAATAATGTTTGGGTTAGTGATCCTAAAA | |
| | | TGGATTAAAGATTAAGTTACTTTAGGGATAACAGCGTAAT | |
| JX514 | Cambarus | AAGGGACGATAAGACCCTATAAAACTTTATATTTAGGGGT | 754 |
| 504 | deweesae | GGAAATTAGTTTTATTTAAAGGTTACTATTTTATAATATT | |
| | | TGGTTGGGGCGACAGGGATAGAAAAAAAGTTAACTGTCTT | |
| | | TTTTTTTTTACAGTAATATTTGGTTGAATGATCCTAAAAT | |
| | | GGATTAAAGATTAAGTTACTTTAGGGATAACAGCGTAAT | |
| JX514 | Cambarus | AAGGGACGATAAGACCCTATAAAACTTTATATTAAAAAGT | 755 |
| 513 | striatus | GATGGGTAAGTTTTGTTTAAAGGTTATTACTTTAAAGTAT | |
| | | TTGGTTGGGGTGACAGGAATAGAATAGAAAGTAACTGTTC | |
| | | TTTTTTTTTTACAGTAGTGTTTGAGTTAGTGATCCTAAAA | |
| | | TGGATTAAAGATTAAGTTACTTTAGGGATAACAGCGTAAT | |
| JX514 | Cambarus | AAGGGACGATAAGACCCTATAAAACTTTATATTGAAAAGT | 756 |
| 506 | graysoni | GGTAGGTATGATTTGTTTAAAAGGTTACTACTTTAAAGTA | |
| | | TTTGGTTGGGGTGACAAAAATAGAATAGAAAGTAACTGTT | |
| | | TTTTTTTTTAACAATAATGTTTGGGTTAGTGATCCTAAAA | |
| | | TGGATTAAAGATTAAGTTACTTTAGGGATAACAGCGTAAT | |
| JX514 | Cambarus | AAGGGACGACAAGACCCTATAAAACTTTATATTGCAAAGT | 757 |
| 537 | monongalensis | GATTAGTAAGTTTTATTTAAAGGTTATTATTTTGAAATAT | |
| | | TTGGTTGGGGTGACAAGGATTGAAGAAAAAGTAACTGTAT | |
| | | TTTTTTTTACAATAATGTTTGGTTTAGTGATACTAAAATG | |
| | | GATTAAAGATTAAGTTACTTTAGGGATAACAGCGTAGT | |
| JX514 | Cambarus | AAGGGACGATAAGACCCTATAAAACTTTATATTTAAAAGT | 758 |
| 511 | pyronotus | GATAGGTAGGTTTTGTTTTAAGGTTATTTCTTTAAAATAT | |
| | | TTGGTTGGGGTGACAGGAATAGAATAGAAAGTAACTGTTC | |
| | | TTTTTTTTTACAGTAGTGTTTGAGTTAGTGATCCTAAAAT | |
| | | GGATTAAAGATTAAGTTACTTTAGGGATAACAGCGTAAT | |
| AF235 | Cambarus | AAGGGACGATAAGACCCTATAAAACTTTATATTGAGAGGT | 759 |
| 988 | maculatue | GAGAGGTAATTTTGTTTAAAAGTTATTGCTTTAAAGTATT | |
| | | TGGTTGGGGCGACAAGGATACAAGGTAAAGTAACTGTTTT | |
| | | TTTTTTCTACAATAATGTTTGAGTGAATGATCCTAAGATG | |
| | | GATTAAAAGATTAAGTTACTTTAGGGATAACAGCGTAAT | |
| JX514 | Cambarus | AAGGGACGATAAGACCCTATAAAACTTTATATTGAGAAGT | 760 |
| 524 | coosawat | AATGGGCAGGTTTTGTTTAGAAGTTATTATTTTTAGAGTA | |
| | tae | TTTGGTTGGGGCGACAAGGATAAAAAGAAAGTAACTGTTC | |
| | | TTTTATTTTACAATAATGTTTGGGTATAATGATCCTAATA | |
| | | TGGATTAAAGATTAAGTTACTTTAGGGATAACAGCGTAAT | |
| JX514 | Cambarus | GAAGGGACGATAAGACCCTATAAAACTTTATATTAAAAAG | 761 |
| 509 | latimanus | TGATGTGTAAGTTTTGTTTAAAGGTTATTACTTTAAAGTA | |
| | | TTTGGTTGGGGTGACAGGAATAGAATAGAAAGTAACTGTT | |
| | | CTTTTTTTTTTACAGTAGTGTTTGAGTTAGTGATCCTAAA | |
| | | ATGGATTAAAGATTAAGTTACTTTAGGGATAACAGCGTAA | |
| | | T | |
| JX514 | Cambarus | AAGGGACGATAAGACCCTATAAAACTTTATATTGAAAAGT | 762 |
| 517 | strigosus | GATAGGTACAATTTGTTTGAAGGGTTGTTATTTTAAAGTA | |
| | | TTTGGTTGGGGTGACAAGGATAAAATGGAAAGTAACTGTT | |
| | | TTTTTTTTTTACAGTGATGTTTGGGTTAATGATCCTAAAA | |
| | | TGGATTAAAGATTAAGTTACTTTAGGGATAACAGCGTAAT | |
| JX514 | Cambarus | AAGGGACGATAAGACCCTATAAAACTTTATACTGAGAGGT | 763 |
| 556 | parrishi | GATGGATGAGTTTTGTTTAGAAGTTATTATTTTAGAGTAT | |
| | | TTGGTTGGGGCGACAAGGGTAAAAAGAAAGTAACTGTTCT | |
| | | TTATTTTTACAGTTATGTTTGAGTGAATGATCCTAAAACG | |
| | | GGTTAAAGATTAAGTTACTTTAGGGATAACAGCGTAAT | |
| JX514 | Cambarus | AAGGGACGATAAGACCCTATAAAACTTTATATTTAGAAGT | 764 |
| 532 | bouchardi | GATAGGTAAGTTTTGTTTGTAGTTTAATTACTTTAAAATA | |
| | | TTTGGTTGGGGTGACAAGGATAGAATAACAAGTAACTGTT | |
| | | TTTTTTTTTAACAATGATGTTTGGATCGGTGATCCTAAAA | |
| | | GGATTAAAAGATTAAGTTACTTTAGGGATAACAGCGTAAT | |
| JX514 | Cambarus | AAGGGACGATAAGACCCTATAAAACTTTATATTAAGAAGT | 765 |
| 525 | fasciatus | AATGGGTGAATTTTGTTTAGAAGTTATTATTTTAAAATAT | |
| | | TTGGTTGGGGCGACAAGGATAAAAAGAAAATAACTGTTCT | |
| | | TTTATTTTACAATAATGTTTGGGTATAATGACCCTAATAG | |
| | | GGGTTAAAAGATTAAGTTACTTTAGGGATAACAGCGTAAT | |
| JX514 | Cambarus | AAGGGACGATAAGACCCTATAAAACTTTATATTGAAAAGT | 766 |
| 508 | harti | GGTAGGTATGGTTTGTTTAAAAGGTTATTGCTTTAAAGTA | |
| | | TTTGGTTGGGGTGACAAGGATAGAATGGAAAGTAACTGTT | |
| | | TTTTTTTTTTACAATGATGTTTGAGCTAATGATCCTAAAA | |
| | | TGGATTAAAGATTAAGTTACTTTAGGGATAACAGCGTAAT | |
| JX514 | Cambarus | AAGGGACGATAAGACCCTATAAAACTTTATATTTGGGGGT | 767 |
| 555 | nerterius | AATAGGTAGGTTTTGTTTGTTAGTTATTATTTTAAAGTAT | |
| | | TTGGTTGGGGTGACAAGGATAAAAAAAAAGTAACTGTTCT | |
| | | TTATTTTTACAATAATGTTTGGTATAATGATCCTAATATG | |
| | | GATTAAAGATTAAGTTACTTTAGGGATAACAGCGTAAT | |
| JX514 | Cambarus | AAGGGACGATAAGACCCTATAAAACTTTATATTTAGGAGT | 768 |
| 539 | setosus | AGAGTTAGTTTTATTTTAATATCTTATTTTAAAGTATTTG | |
| | | GTTGGGGTGACATGAACAAAAAATGAAATAATTGTTTTTA | |
| | | TTTTTTACAATGATATTTGGACTTATGATCCTAAAGTGGA | |
| | | TTAAAGATTAAGTTACTTTAGGGATAACAGCGTAAT | |
| JX514 | Cambarus | AGGGGACGATAAGACCCTATAAAACTTTATATTGAAAAGT | 769 |
| 531 | batchi | GGTAGGTACGATTTGTTTTAAGGTTACTACTTTAAAGTAT | |
| | | TTGGTTGGGGCGACAAGAATAGAATAGAAAGTAGCTGTTT | |
| | | TTTTTATTTTACAATGATGTTTGGGTTAGTGATCCTAAAA | |
| | | CGGATTAAAAGATTAAGTTACTTTAGGGATAACAGCGTAA | |
| | | T | |
| JX514 | Cambarus | AAGGGACGATAAGACCCTATAAAACTTTATATTGAAAAGT | 770 |
| 507 | halli | GGTGGGTATGGTTTGTTTAAAGATTATTACTTTAAAGTAT | |
| | | TTGGTTGGGGTGACAAGAATAGAATAGAAATTAACTGTTT | |
| | | TTTATTTTTACAATGATGTTTGAGTTAGTGATCCTAAAAT | |
| | | GGATTAAAGATTAAGTTACTTTAGGGATAACAGCGTAAT | |
| JX514 | Cambarus | AAGGGACGATAAGACCCTATAAAACTTTATATTTAGAAGT | 771 |
| 533 | crinipes | GATAGGTAAGTTTTGTTTGTAGTTTAATTACTTTAAAATA | |
| | | TTTGGTTGGGGTGACAAGGATAGAATAACAAGTAACTGTT | |
| | | TTTTTTTTTAACAATGATGTTTGGATCGGTGATCCTGAAA | |
| | | GGATTAAAAGATTAAGTTACTTTAGGGATAACAGCGTAAT | |
| JX514 | Cambarus | AAGGGACGATAAGACCCTATAAAACTTTATATTTAGAAGT | 772 |
| 541 | unestami | GGTGGGTAAGTTTTGTTTGTAGTTTAATTACTTTAAAATA | |
| | | TTTGGTTGGGGTGACAAGGATAGAATAGCAAGTAACTGTT | |
| | | TTTTTTTAAAACAATGATGTTTGGATTAATGATCCTAAAG | |
| | | GGATTAAAAGATTAAGTTACTTTAGGGATAACAGCGTAAT | |
| JX514 | Cambarus | AAGGGACGATAAGACCCTATAAAACTTTATATTTGGAAGT | 773 |
| 557 | reburrus | GATGGGTAAGTTTTGTTTGCCGGTTATTATTTTAAAGTAT | |
| | | TTGGTTGGGGTGACAAGGATAAAAAAAAGTAACTGTTCTT | |
| | | TATTTTTTACAATAATGTTTGGTATAATGATCCTAATATG | |
| | | GATTAAAAGATTAAGTTACTTTAGGGATAACAGCGTAAT | |
| AY853 | Cambarus | AGGGACGATAAGACCCTATAAAACTTTATTTGAAAAGTGA | 774 |
| 664 | gentry | TGAGTATGGTTTATTTAAAAGGTTATTATTTTAAAGTATT | |
| | | TGGTTGGGGCGACAAGAACAAAACGGAAAGTAACTGGTTT | |
| | | TTTTTTTTACAATGATGTTTGAGTTAATGATCCTAAAATG | |
| | | GATTAAAGATTAAGTTACTTTAGGGATAACAGCGTAAT | |
| JX514 | Cambarus | AAGGGACGATAAGACCCTATAAAACTTTATATTAGGAAGT | 775 |
| 518 | hubbsi | GATAAGTATATTTTGTTTAAAGAATTATTACTTTAAAGTA | |
| | | TTTGGTTGGGGTGACAAAAATAAAAAAAAAGTAACTGTTT | |
| | | TTTTTTTTAACAATAATGTTTGGGTTAGTGATCCTAAAAT | |
| | | GGATTAAAGATTAAGTTACTTTAGGGATAACAGCGTAAT | |
| DQ411 | Cambarus | AAGGGACGATAAGACCCTATAAAACTTTATATTAAAAAGT | 776 |
| 733 | friaufi | GGTAGGTATGGTTTGTTTGAAGATTATTATTTTTGAGTAT | |
| | | TTGGTTGGGGCGACAGGAATAAAATAAAAGTAACTGTTTT | |
| | | TTTTTTTTACAATGATGTTTGAGTTAATGATCTTAAAATG | |
| | | GATTAAAGATTAAGTTACTTTAGGGATAACAGCGTAAT | |
| JX514 | Cambarus | AAGGGACGATAAGACCCTATAAAACTTTATATTTAGAAGT | 777 |
| 538 | obeyensis | GATAGGTAAGTTTTGTTTGTAGTTTAATTATTTTAAAATA | |
| | | TTTGGTTGGGGTGACAAGGATATAATAGCAAGTAACTGTT | |
| | | TTTTTTTTTAACAATAATGTTTGGGTTGATGATCCTAAAA | |
| | | GGATTAAAAGATTAAGTTACTTTAGGGATAACAGCGTAAT | |
| JX514 | Cambarus | AAGGGACGATAAGACCCTATAAAACTTTATACTGAAAAGT | 778 |
| 522 | cracens | GGTGGGTATGGTTTGTTTTAAAGATTATTACTTTAAAGTA | |
| | | TTTGGTTGGGGTGACAAGAATAAAATAGAAACTAACTGTT | |
| | | TTTTATTTTTACAATGATGTTTGAGTTAGTGATCCTAAAA | |
| | | CGGATTAAAGATTAAGTTACTTTAGGGATAACAGCGTAAT | |
| JX514 | Cambarus | AAGGGACGATAAGACCCTATAAAACTTTATATTTAAAAGG | 779 |
| 530 | asperimanus | AGTAAGCAAATTTTATTTAGAAGTTATTGCTTGAAAATAT | |
| | | TTTGTTGGGGCGACAAGAATAGGGAGGTAAATAACTGTTT | |
| | | TTTTTTTTTGACAATAATGTTTGGATTAGTGATCCTGAAA | |
| | | GGATAATAAGATTAAGTTACTTTAGGGATAACAGCGTAAT | |
| JX514 | Cambarus | CTGTTTTTTTTTTTTACAATAATGTTTGGTTTAGTGATCC | 780 |
| 554 | hobbsorum | TAAAATGGATTAAAGATTAAGTTACTTTAGGGATAACAGC | |
| | | GTAAT | |
| JX514 | Cambarus | AAGGGACGATAAGACCCTGTAAAACTTTATATTTAAGAGT | 781 |
| 523 | williami | GGTGGGTAAGGTTTGTTTGGAGGTAATTATTTTAAAGTAT | |
| | | TTGGTTGGGGCGACAAGAATAGAAAAAAAAATAACTGTTT | |
| | | TTTTTTCTTACAATGATGTTTGAGTTAATGATCCTAAAAC | |
| | | GGATTAAAGATTAAGTTACTTTAGGGATAACAGCGTAAT | |
| JX514 | Cambarus | AAGGGACGATAAGACCCTATAAAACTTTATATTTGGAAGT | 782 |
| 502 | howardi | GATGGTAGATTTTGTTTGTTAGTTATTATTTTAAAGTATT | |
| | | TGGTTGGGGCGACAAGGATAAAAAGAAAGTAACTGTTCTT | |
| | | TGTTTTTACAATAATGTTTGGTTTAGTGATCCTAATATGG | |
| | | ATTAAAAGATTAAGTTACTTTAGGGATAACAGCGTAAT | |
| JX514 | Cambarus | AAGGGACGATAAGACCCTATAAAACTTTATATTAAAAAGT | 783 |
| 510 | obstipus | AATGGGTAAGTTTTGTTTAAAGGTTATTACTTTAAAGTAT | |
| | | TTGGTTGGGGTGACAGGAATAGAATAGAAAGTAACTGTTC | |
| | | TTCTTTTTTACAGTAGTGTTTGAGTTAGTGATCCTAAAAT | |
| | | GGATTAAAGATTAAGTTACTTTAGGGATAACAGCGTAAT | |
| JX514 | Cambarus | AAGGGACGATAAGACCCTATAAAACTTTATATTAAGAAGT | 784 |
| 526 | girardianus | AATGGGTAAATTTTGTTTAGAAGTTATTATTTTAAAGTAT | |
| | | TTGGTTGGGGCGACAAGGATAAAAAGAAAATAACTGTTCT | |
| | | TTCATTTTACAATAATGTTTGGAGATAGTGATCCTAAGAT | |
| | | GGGTTAAAGATTAAGTTACTTTAGGGATAACAGCGTAAT | |
| JX514 | Cambarus | GGGGGACGATAAGACCCTATAAAACTTTATATTGAAAGGG | 785 |
| 534 | cryptodytes | GGTAAGTAAATTTTGTTTAAGGTTATGTGCTTAAAATATT | |
| | | TGGTTGGGGTGACAGGGATAGAAGAAAAGGTAACTGTTTT | |
| | | TTTTTATTACAAATAATATTTGGATTAGTGATCCTGAAGT | |
| | | GGATTTAAGATTAAGTTACTTTAGGGATAACAGCGTAAT | |
| JX514 | Cambarus | AAGGGACGATAAGACCCTATAAAACTTTATATTGAGAAGT | 786 |
| 528 | speciosus | AATGGGCAGGTTTTGTTTAGAAGTTATTATTTTTAGAGTA | |
| | | TTTGGTTGGGGCGACAAGGATAAAAAGAAAGTAACTGTTC | |
| | | TTTTATTTTACAATAATGTTTGGGTATAATGATCCTAATA | |
| | | TGGATTAAAGATTAAGTTACTTTAGGGATAACAGCGTAAT | |
| JX514 | Cambarus | AAGGGACGATAAGACCCTATAAAACTTTATATTAAGAAGT | 787 |
| 503 | sciotensis | AATGGGTAAGTTTTGTTTAAAAGTTATTATTTTAAAGTAT | |
| | | TTGGTTGGGGCGACAAGGATAAAAAGAGAGTAACTGTTCT | |
| | | TTTGTTTTACAATAATGTTTGATATTATGATCCTAAGAAG | |
| | | GATTAAAGATTAAGTTACTTTAGGGATAACAGCGTAAT | |
| JX514 | Cambarus | AAGGGACGATAAGACCCTATAAAACTTTATATTAAGAAGT | 788 |
| 553 | georgiae | AATGGGTGAATTTTGTTTGGAAGTTATTATTTTAGAGTAT | |
| | | TTGGTTGGGGTGACAAGGATAAAAAGAAAGTAACTGTTCT | |
| | | TTTATTTTACAATAATGTTTGGGTTTGATGATCCTAAGAC | |
| | | GGATTAAAGATTAAGTTACTTTAGGGATAACAGCGTAAT | |
| JX514 | Cambarus | AAGGGACGATAAGACCCTATAAAACTTTATATTGAGAAGT | 789 |
| 561 | pristinus | GATAGGTAAGTTTTGTTTATAGGTTAATTACTTTAAAATA | |
| | | TTTGGTTGGGGCGACCGGGATAGAATAACAAGTAACTGTC | |
| | | TTTTTTTAAAACAATTGTGTTTGGATTAGTGATCCTAAGA | |
| | | TGGATTAAAAGATTAAGTTACTTTAGGGATAACAGCGTAA | |
| | | T | |
| JX514 | Cambarus | AAGGGACGATAAGACCCTATAAAACTTTATATTTAGGAGT | 790 |
| 529 | aculabrum | AGAGTTAGTTTTATTTTGGGATCTTATTTTAAAGTATTTG | |
| | | GTTGGGGTGACATGAACAAAAAATGAAATAATTGTTTTTA | |
| | | TTTTTTACAATGATATTTGGGTTTATGATCCTAAAGTGGA | |
| | | TTAAAGATTAAGTTACTTTAGGGATAACAGCGTAAT | |
| JX514 | Cambarus | AAGGGACGATAAGACCCTATAAAACTTTATACTAAAAAGT | 791 |
| 505 | englishi | GATGGGTAAGTTTTGTTTAAAGGTTATTACTTTAAAGTAT | |
| | | TTGGTTGGGGTGACAGGAATAGAATAGAAAGTAATTGTTC | |
| | | TTTTTTTTTACAGTAATGTTTGAGTTAGTGATCCTAAAAT | |
| | | GGATTAAAGATTAAGTTACTTTAGGGATAACAGCGTAAT | |
| DQ411 | Cambarus | AAGGGACGATAAGACCCTATAAAACTTTATATTGAAAAAT | 792 |
| 732 | brachydactylus | GGTAGGTATGGTTTATCTAAGGATCATTATTTTTTAGTAT | |
| | | TTGGTTGGGGCGACAAAAATAGAATAATAAGTAACTGTTT | |
| | | TTTTTTTTAACAATGATGTTTGAGTTAGTGATCCTAAAAT | |
| | | GGATTAGAGATTAAGTTACTTTAGGGATAACAGCGTAAT | |
| JX514 | Cambarus | AAGGGACGATAAGACCCTATAAAACTTTATATTAAAAAGT | 793 |
| 552 | cumberlandensis | AGTGGGTAAATTTTGTTTAGAAATTATTATTTTAAAGTAT | |
| | | TTGGTTGGGGCGACAGGGATAAAAGGAAAGTAACTGTTCT | |
| | | TTTATTTTACAATAATGTTTGGGTACACGATCCTAAGATG | |
| | | GATTAAAGATTAAGTTACTTTAGGGATAACAGCGTAAT | |
| JX514 | Cambarus | AAGGGACGATAAGACCCTATAAAACTTTATATTGAAAGGG | 794 |
| 536 | dubius | GGTAAGTAGATTTTGTTTAAAAGTTATTTCTTAGAAATAT | |
| | | TTGGTTGGGGCGACAAGAATAGGAAGGCAAATAACTGTTT | |
| | | TTTTTTTTTACAATAATTTTTGGGTTAATGATCCTGAAGA | |
| | | GGATTAAAAGATTAAGTTACTTTAGGGATAACAGCGTAAT | |
| JX514 | Cambarus | AAGGGACGATAAGACCCTATAAAACTTTATATTGAAAAGT | 795 |
| 512 | reflexus | GATAGGTATAATTTGTTTAAAAGGTTATTATTTTAAAGTA | |
| | | TTTGGTTGGGGTGACAAGGATANAATGAAAAGTAACTGTT | |
| | | TTTTTTTTTTACAATGATGTTTGGGTTAATGATCCTAAAA | |
| | | TGGATTAAAGATTAAGTTACTTTAGGGATAACAGCGTAAT | |
| JX514 | Cambarus | AAGGGACGATAAGACCCTATAAAACTTTATATTGAAAAGT | 796 |
| 520 | rusticiformis | GATAGGTATATTTTGTTTAAAGAATTATTACTTTAAAGTA | |
| | | TTTGGTTGGGGTGACAAGAATAGAAGAGAAAGTAACTGTT | |
| | | TTTTTTTTATACAATAATGTTTGGGTTAGTGATCCTAAAA | |
| | | TGGATTAAAGATTAAGTTACTTTAGGGATAACAGCGTAAT | |
| JX514 | Cambarus | AAGGGACGATAAGACCCTATAAAACTTTATATTAAGAAGT | 797 |
| 559 | scotti | AGTAGGGTGAATTTTGTTTAGAAGATATTATTTTAAAGTA | |
| | | TTTGGTTGGGGCGACAAGGATAAAAAGAAAATAACTGTTC | |
| | | TTTTATTTTACAATAATGTTTGGGTATAATGATCCTAAGA | |
| | | CGGGTTAAAAGATTAAGTTACTTTAGGGATAACAGCGTAA | |
| | | T | |
| JX514 | Cambarus | AAGGGACGATAAGACCCTATAAAACTTTATATTGAGAAGT | 798 |
| 551 | coosae | AATGGGCAGGTTTTGTTTAGAAGTTATTATTTTTAGAGTA | |
| | | TTTGGTTGGGGCGACAAGGATAAAAAGAAAGTAACTGTTC | |
| | | TTTTATTTTACAATAATGTTTGGGTATAATGATCCTAATA | |
| | | TGGATTAAAGATTAAGTTACTTTAGGGATAACAGCGTAAT | |
| JX514 | Cambarus | AAGGGACGATAAGACCCTATAAAACTTTATATTTAGAAGT | 799 |
| 535 | distans | GATAGGTAAGTTTTGTTTGTAGTTTAATTACTTTAAAATA | |
| | | TTTGGTTGGGGTGACAAGGATAGAATAACAAGTAACTGTT | |
| | | TTTTTTTTTAACAATGATGTTTGGATCGGTGATCCTGAAA | |
| | | GGATTAAAAGATTAAGTTACTTTAGGGATAACAGCGTAAT | |
| JX514 | Cambarus | AAGGGACGATAAGACCCTATAAAACTTTATATTTGGGAGT | 800 |
| 527 | longirostris | GATGGGCAGGTTTTGTTTGTTAGTTATTATTTTAAAGTAT | |
| | | TTGGTTGGGGTGACAAGGATAAAAAGAAAGTAACTGTTCT | |
| | | TTATTTTTACAATAATGTTTGGTATAATGATCCTAATGTG | |
| | | GATTAAAGATTAAGTTACTTTAGGGATAACAGCGTAAT | |
| JX514 | Cambarus | AAGGGACGATAAGACCCTATAAAACTTTATATTGAGAAGT | 801 |
| 519 | hubrichti | GATGGGTATATTTGTTTAAAGATTATTACTTTAAAGTATT | |
| | | TGGTTGGGGTGACAAGAATAGAATAGAAAGTTACTGTTTT | |
| | | TTTTTTTTACAATGATATTTGAGTTGGTGATCCTAAAAGG | |
| | | GATTGAAGATTAAGTTACTTTAGGGATAACAGCGTAAT | |
| MG56 | Monomia | AGGGGACGATAAGACCCTATAAAGCTTTATAAGTCTAGGA | 802 |
| 3792 | lucida | ATTTTAGATATTTTGTAGGATAAATTTATTTTCTGATAAC | |
| | | TTATTTGGTTGGGGCGACAAAGGTATAATTATGGATAACT | |
| | | GCTATTAATTTTGATACATTTATTGGTGATTGAAATTTAG | |
| | | GGTAATTGATCCTCTTTGGGGATTAAAAGATCAAGTTACT | |
| | | TTAGGGATAACAGCGTAAT | |
| MH168 | Faughniaserenei | GAGGGACGATAAGACCCTATAAAGCTTTATAATGTTTTTT | 803 |
| 232 | | TTTATTATAAATTGTGCAATTTTAGTTTACTAAAAAGAAA | |
| | | TTATTATTTTACTGGGGGGGTAAAAGTATGTTCTTGACCA | |
| | | ACTGCTTTTGTTTTTAAAAACAAGTATTTTTGAAAATAAA | |
| | | TTGATCCATTATTAATGATTATAAGAACAAGTTACTTTAG | |
| | | GGATAACAGCGTAAT | |
| MH168 | Harpiosquilla | GAGGGACGATAAGACCCTATAAAGCTTTATAACGTATTTT | 804 |
| 201 | melanoura | TTTTTTATAAACTATTTAATTATTAACTTAATACAAAATG | |
| | | TACGTTATTTTACTGGGGCGGTAAAAGTATAATCAAGAAT | |
| | | AACTGCTTTTTTTATTAATAACAATTATAATTGAAAATAA | |
| | | ATTGATCCATTATTAATGATCATAAGAACAAGTTACTTTA | |
| | | GGGATAACAGCGTAAT | |
| MH168 | Harpiosquilla | GAGGGACGATAAGACCCTATAAAGCTTTATAGTATATTTT | 805 |
| 202 | annandalei | TTTTTTATAAATTATTTTGTTATTAACTTATTATAAAATA | |
| | | TATATTATTTTACTGGGGCGGTAAAAGTATAATTAAGAAT | |
| | | AACTGCTTTTCTTTTTAATAACAGTGATAACTGGAAATAA | |
| | | ATTGATCCATTATTAATGATCACAAGAACAAGTTACTTTA | |
| | | GGGATAACAGCGTAAT | |
| KJ920 | Cherax | GAGGGACGATAAGACCCTATAAAGTTTATACATGGAGTTA | 806 |
| 795 | communis | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTATATGGTCCC | |
| | | TTTTTAGGATTAGAGAGTAAATTACTTTAGGGATAACAGC | |
| | | GTAAT | |
| EU977 | Cherax | GAGGGACGATAAGACCCTGTAAAGTTTGTACATCAGGGTT | 807 |
| 342 | cuspidatus | AATGGGTAGCGATTTAGGTTAATAAGGCTAACTCTTATGG | |
| | | AGGTGTTTAGTTGGGGCGACTGGGATATAAGTTATCTAAC | |
| | | TGTTTCTTTATTGGAATCAATAATATTTGAGTTTATGGTC | |
| | | CTCTTTAAGGATTTCAGAATAAATTACTTCAGGGATAACA | |
| | | GCGTAAT | |
| KJ920 | Cherax | GAGGGACGATAAGACCCTATAAAGTTTATACATGGAGTTA | 808 |
| 829 | paniaicus | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTATATGATCCC | |
| | | TTTTTAGGATTAGAGAGTAAATTACTTTAGGGATAACAGC | |
| | | GTAAT | |
| KJ920 | Cherax | GAGGGACGATAAGACCCTATAAAGTTTATACATGAAGCTG | 809 |
| 812 | lorentzi | GTTAAGAGTGATTTAGGGTGATAAAGTTTATTATTAGCAG | |
| | | GGTGTTTAGTTGGGGCGACTGAGATATAAATCATATAACT | |
| | | GTTTTTTGTTTAAATCAGAGATACTTGCTGGTATTGGTCC | |
| | | CTTTTTAGGATTAGAGAAAAAATTACTTTAGGGATAACAG | |
| | | CGTAAT | |
| KJ920 | Cherax | GAGGGACGATAAGACCCTATAAAGTTTATACATGAAGCTG | 810 |
| 772 | albertisii | GTTAAGAGTGATTTGAGGTATTAAAGTTTATTATCAGCAG | |
| | | GGTGTTTAGTTGGGGCGACTAGGATATAAATTATATAACT | |
| | | GTTTTTTGTTTAAATCAGAGATACTTGTTTATATGATCCC | |
| | | TTTTTAGGATTAGAGAATAAATTACTTTAGGGATAACAGC | |
| | | GTAAT | |
| AF135 | Cherax | GAGGGACGATAAGACCCTATAAAGTTTTGACACTAAATTA | 811 |
| 973 | rotundus | ATTAAAGGTGATTTGGGTTATAAAGTTTTATTATTAAATA | |
| | | AGTGTTTAGTTGGGGCGACTAGGATATAAGTGATTTAACT | |
| | | GTTTCTTCGTTTAAACCAAAAATATTTGGGTTTATGATCC | |
| | | TTTTTTAGGATTTTTAGAGTAAATTACTTTAGGGATAACA | |
| | | GCGTAAT | |
| KM039 | Cherax | GAGGGACGATAAGACCCTGTAAAGTTTGTACATCAGGGTT | 812 |
| 077 | leckii | AATGGGTAGAGATTTAGGTTAATAAGGCTAACTCTTATGG | |
| | | GGGTGTTTAGTTGGGGCGACTAGGATATAAGTTATCTAAC | |
| | | TGTTTCTTTGTTGGAATCAATAATATTTGAGTTTATGATC | |
| | | CTCTTTAAGGATTTCAGAATAAATTACTTCAGGGATAACA | |
| | | GCGTAAT | |
| KJ920 | Cherax | GAGGGACGATAAGACCCTATAAAGTTTATACATGGAGTTA | 813 |
| 825 | murido | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTGTATGGTCCC | |
| | | TTTTTAGGATTAGAGAGTAAATTACTTTAGGGATAACAGC | |
| | | GTAAT | |
| KM039 | Cherax | GAGGGACGATAAGACCCTATAAAGTTTGTACAGTGAATTA | 814 |
| 082 | wasselli | GTATGAAATAGATTAGATGGGTAAAATTTGTTACTAAGGA | |
| | | GATGTTTGGTTGGGGCGATTAGAATATAGGTGAAGTAACT | |
| | | GTTTCTTTTTTTGAGTCAAAAATTTTTGATTTAATGACCC | |
| | | TTAATAAGGATTATGAAAGCAAGTTACTTTAGGGATAACA | |
| | | GCGTAAT | |
| KM039 | Cherax | GGGGGACGATAAGACCCTATAAAGTTTGTACATTAGGCTA | 815 |
| 078 | parvus | ATTAAGAGTGATTTAGATTTATAAGGCTTTATTATTAGGG | |
| | | GGTGTTAAGTTGGGGCGACTAGGATATAATTTATATAACT | |
| | | GTTTCTTTATTATAATCAGAAATATTTGAGTTTATGATCC | |
| | | TTTTTTAGGATTTTTAGAGTAAATTACTTTAGGGATAACA | |
| | | GCGTAAT | |
| KJ920 | Cherax | GAGGGACGATAAGACCCTATAAAGTTTATACATAGAGTTA | 816 |
| 828 | pallidus | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTATATGGTCCC | |
| | | TTTTTAGGATTAGAGAGTAAATTACTTTAGGGATAACAGC | |
| | | GTAAT | |
| KM039 | Cherax | GAGGGACGATAAGACCCTATGAAGTTTGTACATTGGATTA | 817 |
| 079 | cartalacoolah | ATTGGGAATGATTTATGTTTAGTAAGGTTTGCTATTGAGA | |
| | | AGATGTTTAGTTGGGGCGACTAGGATATAATATATAACTG | |
| | | TTTCTTGGTAGGAGTCAAAAATATTTGAGTTAATGATCCT | |
| | | TTTTTAGGATTTTAGAGTAAATTACTCTAGGGATAACAGC | |
| | | GTAAT | |
| KJ920 | Cherax | GAGGGACGATAAGACCCTATAAAGTTTATACATGGAGTTA | 818 |
| 806 | longipes | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTATATGGTCCC | |
| | | TTTTTAGGATTAGAGAGTAAATTACTTTAGGGATAACAGC | |
| | | GTAAT | |
| KJ920 | Cherax | GAGGGACGATAAGACCCTATAAAGTTTATACATGGGGCTG | 819 |
| 765 | rhynchotus | GTTGAGAGTGATTCAGAATAGTAAAGTTTATTATTAGCAA | |
| | | GATGTTTAGTTGGGGCGACTAAGATATAAATTATATAACT | |
| | | GTCTTTTGTTTAAATCAGAGGTGTCTGTTCGTATGATCCT | |
| | | TTTTTAGGATTGGAGAGTAAATTACTTTAGGGATAACAGC | |
| | | GTAAT | |
| KY654 | Cherax | GAGGGACGATAAGACCCTATAAAGTTTATACATAGGGTTA | 820 |
| 091 | pulcher | GTTAAAAGTGATTTAGGATAGTAAAGTTTGTTACTAGCAA | |
| | | GATGTTTGGTTGGGGTGACTAGAATATAAGTTATATAACT | |
| | | GTTTTTTATTTAAGTCAGAAGTATTTGTTTATAGTGATCC | |
| | | CTTATTTAGGATTAAAGAGTAAATTACTTTAGGGATAACA | |
| | | GCGTAAT | |
| KJ920 | Cherax | GAGGGACGATAAGACCCTATAAAGTTTATACACAGAATTA | 821 |
| 835 | peknyi | ATCGAAAATAATTTGGAATAATAAAATTTCTTATTAGTGA | |
| | | GGTGTTTGGTTGGGGCGACTAGGATATAAGTTATATAACT | |
| | | GTTTTTTGTTTAAATCAAAGATATTTGTTTATATGATCCC | |
| | | TTTTTAGGATTAAAGAGTAAATTACTTTAGGGATAACAGC | |
| | | GTTAT | |
| AF135 | Cheraxsetosus | GAGGGACGATAAGACCCTATAAAGTTTGACATTAAATTAA | 822 |
| 972 | | TTAAAGGCGATTTAGGTCGTTAAAGTCTTATTATTATATA | |
| | | AGTGTTTAGTTGGGGCGACTAGGATATAAGTGATTTNNAA | |
| | | CTGTTTTCTTCGTTCGAGTCAAGAATATTTGAGTGTATGA | |
| | | TCCTTTTTTAGGATTTTAGAGTAAATTACTTTAGGGATAA | |
| | | CAGCGTAAT | |
| KJ920 | Cherax | GAGGGACGATAAGACCCTATAAAGTTTATACATGGAGTTA | 823 |
| 813 | misolicus | GTTAAGAGTAATTTAGAATAGTAAAGTTTATTATTAACAA | |
| | | GATGTTTGGTTGGGGCGACTGGAATATAAGTTATATAACT | |
| | | GTTTTTTATTTAAGTCAGAAATATTTGTTTATAATGATCC | |
| | | TTTTTTTGGATCAAAGAGTAAATTACTTTAGGGATAACAG | |
| | | CGTAAT | |
| KY654 | Cherax | GAGGGACGATAAGACCCTATAAAGTTTTATACATGGAGTT | 824 |
| 088 | warsamsonicus | AGTTAAGAGTAATTTAGAGTAGTAAAGTTTATTATTAGCA | |
| | | AGATGTTTGGTTGGGGCGACTGGAATATAAGTTATATAAC | |
| | | TGTTTTTTATTTAAGTCAGAAATATTTGTTTATAATGATC | |
| | | CCTTTTTTGGATTTAAGAGTAAATTACTTTAGGGATAACA | |
| | | GCGTAAT | |
| KJ920 | Cheraxsolus | GACGATAAGACCCTATAAAGTTTATACATGGAGTTAATCA | 825 |
| 836 | | AGAGTGATTTGGAATAGTAAAGTTCATTATTAGCAAGATG | |
| | | TTTAGTTGGGGTGACTAGAATATAAGTTATATAACTGTTT | |
| | | TTTGTTTTAATCAGAGATATCTGTTTATATGGTCCCTTTT | |
| | | TAGGATTAGAGAGTAAATTACTTTAGGGATAACAGCGTAA | |
| | | T | |
| KY654 | Cheraxsnowden | GAGGGACGATAAGACCCTATAAAGTTTAAACACTGAGTTA | 826 |
| 087 | | GTTAAGAGTGATTTAGAGTAGTAAGGTTTATTATTAGTTA | |
| | | GGTGTTTGGTTGGGGCGACTAGAATATAAGTTATATAACT | |
| | | GTTTTTTATTTGAATCAGAGATATCTGTTCACATGATCCC | |
| | | TTTTTAGGATTAAAGAGTAAATTACTTTAGGGATAACAGC | |
| | | GTAAT | |
| KJ920 | Cherax | GAGGGACGATAAGACCCTATAAAGTTTATACATGGAGTTA | 827 |
| 785 | buitendijkae | ATCAAGAGTGATTTGGAATAGTAAAGTTCATTATTAGCAA | |
| | | GATGTTTAGTTGGGGTGACTAGAATATAAGTTATATAACT | |
| | | GTTTTTTGTTTTAATCAGAGATATCTGTTTATATGGTCCC | |
| | | TTTTTAGGATTAGAGAGTAAATTACTTTAGGGATAACAGC | |
| | | GTAAT | |
| KJ920 | Cherax | GAGGGACGATAAGACCCTATAAAGTTTATACATGGAGTTA | 828 |
| 784 | boschmai | ATCAAGAGTGATTTGGAATAGTAAAGTTCATTATTAGCAA | |
| | | GATGTTTAGTTGGGGTGACTAGAATATAAGTTATGTAACT | |
| | | GTTTTTTGTTTTAATCAGAGATATCTGTTTATATGATCCC | |
| | | TTTTTAGGATTAGAGAGTAAATTACTTTAGGGATAACAGC | |
| | | GTAAT | |
| KJ920 | Cherax | GGGGGACGATAAGACCCTATAAAGTTTGTACATGGGGCCG | 829 |
| 760 | nucifraga | CTTAGGAGCGATTTAGAGAAGTAAAGTTTATTACTAGCAA | |
| | | GGTGTTTCGTTGGGGCGACTAAGATATAAATTATATAACT | |
| | | GTTTTCTGTTAGAATCAGAGATGTCTGTACATATGATCCC | |
| | | TTTTTAGGATGAAAGAGTAAATTACTTTAGGGATAACAGC | |
| | | GTAAT | |
| KJ920 | Cherax | GAGGGACGATAAGACCCTATAAAGTTTATACATGGGGCTG | 830 |
| 752 | barrette | GTTGAGAGTGATTCAGAATAGTAAAGTTTATTATTAGCAA | |
| | | GATGTTTAGTTGGGGCGACTAAGATATAAATTATATAACT | |
| | | GTCTTTTGTTTAAACCAGAGGTATCTGTTCGTATGATCCT | |
| | | TTTTTAGGATTGGAGAGTAAATTACTTTAGGGATAACAGC | |
| | | GTAAT | |
| MH168 | Oratosquilla | GAGGGACGATAAGACCCTATAAAGCTTTATGGTATTTTTC | 831 |
| 227 | fabricii | TTTTTTATAAATTATGTTATTATTAACTTATTTTAGAACT | |
| | | AATATTATTTTACTGGGGCGGTAAAAGTATAATAAAGACT | |
| | | AACTGCTTTTGTTTTGTATAACAATTATAATTGAAAATAA | |
| | | ATTGATCCATTATTAATGATCACAAGAACAAGTTACTTTA | |
| | | GGGATAACAGCGTAAT | |
| DQ006 | Astacopsis | GGGGGACGATAAGACCCTATAAAGTTTAACATAATAATGA | 832 |
| 549 | tricornis | TAAAAAATTAATTAGGTTATAAAGTTTATTATTACAAATA | |
| | | ATGTTTTGTTGGGGCGACAAGAATAAAAATAATTTAACTG | |
| | | TTCTTTTTTTTATCAAAAATATTTGGGTGGGTGATCTTTT | |
| | | TTAAAAGTATTAGAGTAAATTACTTTAGGGATAACAGCGT | |
| | | AAT | |
| FJ152 | Thalamita | GAGGGACGATAAGACCCTATAAAGCTTTATAAGTTTAGGT | 833 |
| 163 | admete | ATTCTATAGATTTCCACATAAAAATAGATTTTCAAATGCT | |
| | | TATTTTGTTGGGGCGACAATGGTACAATAATAGTAACTGC | |
| | | TTTTAAGTATAACATATATGGATGAGTCATATTTAATTGA | |
| | | TCCTTTCTAAAGATTTTAAGATCAAGTTACTTTAGGGATA | |
| | | ACAGCGTTAT | |
| KT959 | Faxonius | AGGGGACGATAAGACCCTATAAAACTTTATATTTTAAGGT | 834 |
| 518 | virilis | GGAAGATAATTTTATTTTAAAATTACTATTTTAAATTATT | |
| | | TTGTTGGGGCGACAAGGATATAAGAAAGGGTAACTGTCTT | |
| | | TTATTTTTACAATAATATTTGAATTATTGATCCTAAAAGG | |
| | | GATTAAAGATTAAGTTACTTTAGGGATAACAGCGTAAT | |
| KJ132 | Thranita | AAGGGACGATAAGACCCTATAAAGCATTTGGTTGGGGCGA | 835 |
| 641 | prymna | CAATAGTATAATTAATAACTGCTTTAAAATAATACAAATA | |
| | | TATTTGAAGTTGATCCTTTATAAAGATCTAAGATCAAGTT | |
| | | ACTTTAGGGATAACAGCGTTAT | |
| AF044 | Astacopsis | GGGGGACGATAAGACCCTATAAAGTTTAACATAATAAGGA | 836 |
| 240 | franklinii | TAAAAAATTAATTAGGTTTATAAAGTTTATTATTACAAAT | |
| | | AATGTTTTGTTGGGGCGACAAGAATAAAAGTAATTTAACT | |
| | | GTTCTTTTTTTAATCAAAAATATTTGGGTGGGTGATCTTT | |
| | | TCTAAAAGTATTAGAGTAAATTACTTTAGGGATAACAGCG | |
| | | TAAT | |
| KX268 | Cambaroi | GAGGGACGATAAGACCCTATAAAACTTTATATACTTATAA | 837 |
| 737 | desschrenckii | TTTAATAGAAATCTTTGTTTATAAAAATTTAGATTAGAGT | |
| | | ATTTTATTGGGGCGATGAAAATAAAATCTTAGGTAACTGT | |
| | | TTTTGTTTTTTAACAATAATATTTGAATTTTAGATCCCGA | |
| | | AAAAGATCAAAAGATTAAGTTACTTTAGGGATAACAGCGT | |
| | | TAT | |
| EU433 | Orconectes | GAGGGACGATAAGACCCTATAAAACTTTATATTAAAAAGT | 838 |
| 912 | australis | GATGAGTGTGTTTTGTTTAAGGGTTATTATTTTAAAGTAT | |
| | | TTGGTTGGGGCGACAAAAATAGAAGGGAAAGTAACTGTTT | |
| | | TTTTTTATTACAATAATGTTTGAGTGGATGATCCTAAAGT | |
| | | GGATTAAAGATTAAGTTACTTTAGGGATAACAGCGTAAT | |
| LC469 | Thalamita | GCTTTATAANTTTAGGGAGATCTTATAAATATTTAAGTAA | 839 |
| 670 | chaptalii | CAATAATTTTTTAAATGTTTATTCGGTTGGGGCGACAAGA | |
| | | GTATAACGAAAATAACTGCTTTAAATTAAGACAGAAATAT | |
| | | CTGAATCATAGTTAATTGATCCTTTTTAAAGATTCTAAGA | |
| | | TCAAGTTA | |
| LC469 | Zygita | GCTTTATAAATTTAGAGATCTTATATATTTTTATATAGCT | 840 |
| 673 | longifrons | GTAATTTCTTAAATGTTTATTTGGTTGGGGCGACAATAGT | |
| | | ATAACAGAAGTAACTGCTTTTAGGTAGTACAAGCTTATTT | |
| | | GAGTAATACCGTTAATTGATCCTTTATAAAGATTTAAAGA | |
| | | CCAAGTT | |
| LC469 | Thalamita | GCTTTATAAGTTTAAAGATTTTGTATATTTTATTATAGCT | 841 |
| 671 | picta | ATAATTTCTTGAATACTTATTTAATTGGGGCGATAACAGT | |
| | | ATAATTATAGTAACTGCTTGTAAGTAATACAAAAATACTT | |
| | | GAATTGAAATTATAATTGATCCTTTATAAAGATTTTAAGA | |
| | | TCAAGTT | |
| LC469 | Thalamitaseurati | GCTTTACAAGTTTAAAGATCTTATATATTTTTCTATATCA | 842 |
| 672 | | ATAATTTCTTGATTATTTGTTTGGTTGGGGCGACGATAGT | |
| | | ATAATAATAGTAACTGCTTTAAAGTGTTTCAAATGTATTT | |
| | | GAATCGAAATAATAACTGNATCCTTAAATAAAGATTTAAA | |
| | | GATCAAGTT | |
| LC430 | Thranita | GCTTTATAAGTTTGGAGATTTTATATATTTTTCTATAGCA | 843 |
| 787 | pelsarti | ATAATTTCTTGAAAGCTTATTTGGTTGGGGCGACAATAGT | |
| | | ATAATTGAAATAACTGCTTTAAAATAATACAAATATATTT | |
| | | GAATTATAATTAGTTGATCCTTTATAAAGATTTAAGATCA | |
| | | AGTT | |
| EU433 | Orconectes | GAGGGACGATAAGACCCTATAAAACTTTATATTGAAAAGT | 844 |
| 913 | barri | GATGGGTATGTTTTGTTTAGGGATTATTATTTTAAAGTAT | |
| | | TTGGTTGGGGCGACAAAAATAGAAGAGAAAGTAACTGTTT | |
| | | TTTTTTATTACAATAATGTTTGAGTTGATGATCCTAAAAT | |
| | | GGATTAAAGATTAAGTTACTTTAGGGATAACAGCGTAAT | |
| JX514 | Faxonius | ATGGGACGATAAGACCCTATAAAACTTTATATTTCAAGGT | 845 |
| 565 | ronaldi | AGAAAATTAATTTTATTTTAAGGTTACTATTTTAAAATAT | |
| | | TTTGTTGGGGCGACAAGGGTATAAGAAATGATAACTGTCT | |
| | | TTTATTTTTACAATAATATTTGATTTATTGATCCTAAAAG | |
| | | GGATTAAAGATTAAGTTACTTTAGGGATAACAGCGTAAT | |
| JX514 | Faxonius | AGGGGACGATAAGACCCTATAAAACTTTATATTTTAAGGT | 846 |
| 564 | neglectus | GGAAATTAATTTTATTTTGAGGATTACTATTTTAAAATGT | |
| | | TTCGTTGGGGCGACAAGGATATAAGAAAGGGTAACTGTCT | |
| | | TTTGGTTTAACAGTAATATTTGGTTTGTTGATCCTAAAAA | |
| | | GGATTAAAGATTAAGTTACTTTAGGGATAACAGCGTGAT | |
| EU433 | Orconectes | AAGGGACGATAAGACCCTATAAAACTTTATATTAAAAAGT | 847 |
| 917 | compressus | AGAAGTTAATTTTATTTGAGTGTTACTATTTAAAATATTT | |
| | | GGTTGGGGCGATCGGAATAAAAGGTCTAATAACTGTCCTT | |
| | | TTTTTTTACAATAATATTTGGTTTAATGATCCTAAAAGGG | |
| | | ATTTAAGATTAAGTTACTTTAGGGATAACAGCGTAAT | |
| EU433 | Orconectes | AAGGGACGATAAGACCCTATAAAACTTTATATTTTGAGGT | 848 |
| 919 | forceps | AGAAATTAATTTTATTTCATATCACTATTTTAAAGTATTT | |
| | | CGTTGGGGCGACAAGGATACAAGAAAAGGTAACTGTCTTT | |
| | | TGTTTTTACAAAAATATTTGGTTTGTTGATCCTTAAAGGG | |
| | | ATTAAAGATTAAGTTACTTTAGGGATAACAGCGTAAT | |
| EU433 | Orconectes | AAGGGACGATAAGACCCTATAAAACTTTATATTGAAAAGT | 849 |
| 914 | pellucidus | GGTGGGTATGTTTTATTTTGAGGTTATCACTTAAAAGTAT | |
| | | TTGGTTGGGGTGACAAAAATAGAAGGAAAAGTAACTGTTT | |
| | | TTTTTTCTTACAGTGATGTTTGGGTTAGTGATCCTAAAAT | |
| | | GGATTAAAAGATTAAGTTACTTTAGGGATAACAGCGTAAT | |
| NC_04 | Neoeriocheir | ATGGGACGATAAGACCCTATAAAGCTTAATATTAAAATTT | 850 |
| 1211 | leptognathus | TATTTGATTAAATTTTTTTTCAATAAAAATCTAATAATTA | |
| | | AATTTATTTTATTGGGGCGATAAAAGTATAATGTTTGATA | |
| | | ACTGCTTAATTTTAATTACACCTATGCGTGAATTAATTTA | |
| | | TGATTGATCCTAATTAAAGATTAAAAGTTTAAGTTACTTT | |
| | | AGGGATAACAGCGTTAT | |
| AF279 | Penaeus | GGGGGACGATAAGACCCTATAAAGCTTGACAATAATCTTA | 851 |
| 826 | kerathurus | TTAAATTATAAATTGTTAGTATAACTTGATTTTAATTAGA | |
| | | GTTTGTTTCGTTGGGGCGACGAGAATATAATAAATAACTG | |
| | | TTCTTTTAAATATAATTACAAAAATAATTGGTAAAATATT | |
| | | GATCCTTTATTAAAGATTAAAAGATTAAGTTACTTTAGGG | |
| | | ATAACAGCGTAAT | |
| AF279 | Penaeus | GGGGGACGATAAGACCCTATAAAGCTTGACAATAATCCTG | 852 |
| 824 | marginatus | CTAAATTATAAATTGTTAGTATAACTTAATTCTAGCTAGA | |
| | | ATTTGTTTCGTTGGGGCGACGGGAATATAATAAATAACTG | |
| | | TTCTTTTAAATATAGTTACAGAAATGTCTGCTATAAGATT | |
| | | GATCCTTTATTAAAGATTAAAAGATTAAGTTACTTTAGGG | |
| | | ATAACAGCGTAAT | |
| AF279 | Penaeus | GGGGGACGATAAGACCCTATAAAGCTTGACAATAACTTCG | 853 |
| 823 | longistylus | TTAAATTATAAATTGTTAGTATAACTTGATTTTAACTGGG | |
| | | GTTTGTTTCGTTGGGGCGACGAGAATATAATAAAATAACT | |
| | | GTTCTTTTAAATATAATTACAAAAATGCTTGGTGGATAAT | |
| | | TGATCCTTTAATAAAGATTAAAAGATTAAGTTACTTTAGG | |
| | | GATAACAGCGTAAT | |
| AF279 | Penaeus | GGGGGACGATAAGACCCTATAAAGCTTGACAATAACTTTG | 854 |
| 822 | plebejus | TTAAATTATAAATTGTTAGTATAACTTGATTTTAACTAGG | |
| | | GTTTGTTTCGTTGGGGCGACGAGAATATAATAAATAACTG | |
| | | TTCTTTTAAATATAATTACAAAAATGCTTGGTGGATGATT | |
| | | GATCCTTTATTAAAGATTAAAAGATAAAGTTACTTTAGGG | |
| | | ATAACAGCGTAAT | |
| AF105 | Metapenaeopsis | AGAGGGACGATAAGACCCTATAAAGCTTTACAATTATTTA | 855 |
| 044 | liui | GTTAAATTATAAATTGTTGGTATAACTTGATTTAATTAAT | |
| | | ATTTGTTTCGTTGGGGCGACGAGAATAAAATTAAAATAAC | |
| | | TGTTCTTTTATTAGTAGTTACAAATTTGTTTGGTTAAAAA | |
| | | TTGATCCTTTATTAGAGATCAAAAGATTAAGTTACTTTAG | |
| | | GGATAACAGCGTAAT | |
| AF105 | Metapenaeopsis | AAGGGACGATAAGACCCTATAAAGCTTTACAATTATTTAA | 856 |
| 043 | lamellata | TTAAATTATAAATTATTGGTTTAACTTAATTTAATTAATA | |
| | | TTTGTTTCGTTGGGGCGACGAGAATAAAATTAAAATAACT | |
| | | GTTCTTTTATAATTAATTACAAATTTGTTTGGTTAAATAT | |
| | | TGATCCTTTATTAAAGATTAAAAGATTAAGTTACTTTAGG | |
| | | GATAACAGCGTAAT | |
| AF105 | Metapenaeopsis | AAGGGACGATAAGACCCTATAAAGCTTTACAATTATTTAG | 857 |
| 040 | acclivis | TTAAATTATAAATTGTTGGTTTAACTTGATTTAATTAATA | |
| | | TTTGTTTCGTTGGGGCGACGAGAATAAAATTTAAGTAACT | |
| | | GTTCTTTTATAAATAGTCACAAATTTATTTGGTTAGAAAT | |
| | | TGATCCTTTCTTAAAGATCAAAAGATTAAGTTACTTTAGG | |
| | | GATAACAGCGTAAT | |
| AF105 | Metapenaeopsis | AAGGGACGATAAGACCCTATAAAGCTTTACAATTATTTGG | 858 |
| 042 | commensalis | CTAATTTATAAATTGTTGGTTTAACTTAAGTGAGTCAATG | |
| | | TTTGTTTCGTTGGGGCGACGAGAATAAAATTAAGTAACTG | |
| | | TTCTTTAAAAGTAATTACAAAGTTAATTGATAAATAATTG | |
| | | GTCCTTTACTAAAGATTAAAAGATTAAGTTACTTTAGGGA | |
| | | TAACAGCGTAAT | |
| AY622 | Atypopen | GTGGGACGATAAGACCCTATAAAGCTTTACAATTATTTGA | 859 |
| 201 | aeusstenodactylus | TTAAATTATAAATTGTTAGTATAAACTTAATTTAGTTAAT | |
| | | ATTTGTTTTGTTGGGGCGACGAGAATATAATTTTAATAAC | |
| | | TGTTCTTTTATAATAATTTACTTTGATAATTGGAGAATAA | |
| | | TTGATCCTTTACTAAAGATTAAAAGATTAAGTTACTTTAG | |
| | | GGATAACAGCGTAAT | |
| AY601 | Aristeus | AGGGGACGATAAGACCCTATAAAGCTTTACGATTTGTTTA | 860 |
| 738 | antillensis | TTAAATCAAAAATTGTAAGTATAAATTTTATTTAATAGAC | |
| | | ATTTGTTTCGTTGGGGCGACGAGAATAAAATTTAGTAACT | |
| | | GTTCTTTTAGAGTATAACAATTATAATTGAAGGAAAATTG | |
| | | ATCCCTTATTAAGGATTAAAAATTTAAGTTACTTTAGGGA | |
| | | TAACAGCGTAAT | |
| AY601 | Solenocera | GGGGGACGATAAGACCCTATAAAGCTTTACAATAAGTTGG | 861 |
| 736 | vioscai | TTGAATTATAAATTGTTAGTTTAACTTAATTTAATTGGTG | |
| | | TTTGTTTCGTTGGGGCGACGAGAATATAATAATATAACTG | |
| | | TTCTTTTGAAAATTTAACAAAGTTGATTGGATAATAATTG | |
| | | ATCCTTTAATAAAGATTAAAAGATTAAGTTACTTTAGGGA | |
| | | TAACAGCGTAAT | |
| NC_05 | Trachysal | GGGGGACGATAAGACCCTATAAAGCTTTACAATTATTTAG | 862 |
| 0695 | ambria | TTAAATTATAAATTGTTAGTATAAACTTAATTTAATTGAT | |
| | curvirostris | GTTTGTTTCGTTGGGGCGACGAGAATATAATTTTGTAACT | |
| | | GTTCTTTTATAGTTTGTTACAATTATTTTTGGTAAAAGAT | |
| | | TGATCCTTTATTAAAGATTAAAAGATTAAGTTACTTTAGG | |
| | | GATAACAGCGTAAT | |
| AY264 | Penaeus | AGGGGACGATAAGACCCTATAAAGCTTGACAATAATTTAA | 863 |
| 908 | chinensis | TTATACTATCAATTGTTAGTGTAACTTGGTTTTAATTAAT | |
| | | ATTTGTTACGTTGGGGCGACGGGAATATAACAAGTAACTG | |
| | | TTCTTAAATATTTAATAACAAATATAATTGGAAAACTAGC | |
| | | ATGATCCTCTATTAGTGATTAAAAGATTAAGTTACTTTAG | |
| | | GGATAACAGCGTAAT | |
| AY264 | Penaeus | GGGGGACGATAAGACCCTATAAAGCTTGACAATAACTTCG | 864 |
| 907 | canaliculatus | TTATATTATAAATTGTTAGTATAACTTGATTTTAACGGGG | |
| | | GTTTGTTTCGTTGGGGCGACGGGAATATAATAAATAACTG | |
| | | TTCTTTTAAATATAATTACAAAAATGTTTGGTAAATAATT | |
| | | GATCCTCTATTAGAGATTAAAAGATTAAGTTACTTTAGGG | |
| | | ATAACAGCGTAAT | |
| NC_04 | Metapenaeopsis | AAGGGACGATAAGACCCTATAAAGCTTTACAATTATTTAA | 865 |
| 0139 | barbata | TTAAGTTATAAATTGTTGGTTTAACTTAACTTAGTTAATA | |
| | | TTTGTTTCGTTGGGGCGACGAGAATAAAATTAAGTAACTG | |
| | | TTCTTTTGTAGTTAATTACAAATTTGTTTGGTAGAAAATT | |
| | | GATCCTTTATTAAAGATTAAAAGATTAAGTTACTTTAGGG | |
| | | ATAACAGCGTAAT | |
| AF279 | Penaeusesculentus | AGGGGACGATAAGACCCTATAAAGCTTGACAGTAAGTTGA | 866 |
| 828 | | TTATATTATAAATTGTTAGTATAACTTGATTTTAGTTAGC | |
| | | GTCTGTTACGTTGGGGCGACGAGAATATAATAAGTAACTG | |
| | | TTCTTAAGTATCTAATAACAACTATAGTTGGTAAATAAAT | |
| | | GATCCTCTATTAGGGATTAGAAGATTAAGTTACTTTAGGG | |
| | | ATAACAGCGTAAT | |
| AY622 | Heteropenaeus | GGGGGACGATCAGACCCTATTAAGCTTGACTAGTATTTAT | 867 |
| 202 | longimanus | TTAAATTACAAACTGTCAGTATAACGTAATCTTAAATAAA | |
| | | AGTTGTTTCGTTGGGGCGACGGGAATATAAACAATAACTG | |
| | | TTCTTTTGATTATCTTACAAAAATAATTGGTAAATAAGTG | |
| | | ATCCTTTATTAAAGATTAAAAGATTAAGTTACTTTAGGGA | |
| | | TAACAGCGTAAT | |
| EF601 | Atypopenaeus | GAGTGACGATAAGACCCTATAAAGCTTTACAATTATTTGG | 868 |
| 684 | dearmatus | TTGAATTATAAATTATTAGTATTAACTTAATTCAATTAAT | |
| | | ATTTGTTTCGTTGGGGCGACGGGAATATAATTTTAGTAAC | |
| | | TGTTCTTTTATAATTAATTACTTTTATTATTAGGAAAATA | |
| | | ATTGATCCTTTATTAAAGATTAAAAGATTAAGTTACTTTA | |
| | | GGGATAACAGCGTAAT | |
| AY622 | Funchalia | AGGGGACGATAAGACCCTATAAAACTTAACAATAATTTAA | 869 |
| 218 | taaningi | TTAAATTATAAATTGTAAGTGTAACTTAATTTTAATTAAT | |
| | | ACTTGTTTCGTTGGGGCGACGAGAATATAAAAAATAACTG | |
| | | TTCTTTTAAATATTTACATGTGTATAGTTGGTAAAAATTG | |
| | | ATCCTTTATTAAAGATTAAAAGATTAAGTTACTTTAGGGA | |
| | | TAACAGCGTAAT | |
| AY622 | Xiphopenaeus | AGGGGACGATAAGACCCTATAAAGCTTTACAAATATCTAG | 870 |
| 217 | kroyeri | TTAATTTATAAATTGTTATATAAACTTAGGTTAATTAGTG | |
| | | CTTGTTTCGTTGGGGCGACGAGAATATAATTTTGTAACTG | |
| | | TTCTTTTATGGTTTAGTACAATAATTTTTGGTAAAAAATT | |
| | | GATCCTTTATTAAAGATTAAAAGATTAAGTTACTTTAGGG | |
| | | ATAACAGCGTAAT | |
| AY622 | Trachypenaeopsis | AAGGGACGATAAGACCCTATAAAGCTTTACAAACACTTAG | 871 |
| 216 | mobilispinis | CTGGATTTTGAAATGTTAGTATAAATTTAATTCAGTTAAA | |
| | | GATTGTTCCGTTGGGGCGACGAAAATATAAATTTAAGTAA | |
| | | CTGTTTTGAAAATAAATTACAGATATATCTGAAAATAAAT | |
| | | GATCCTTTAATAAAGATTAAGAGATCAAGTTACTTTAGGG | |
| | | ATAACAGCGTAAT | |
| AY622 | Rimapen | AGGGGACGATAAGACCCTATAAAGCTTTACAATTATCTTG | 872 |
| 215 | aeussimilis | TTAAATTATAAATTGTTAGTTTAATCTTAATTTAATTAGT | |
| | | GTTTGTTTCGTTGGGGCGACGGGAATATAATGTTTTGTAA | |
| | | CTGTTCTTTTATAATTTGTTACAATTAATGTTTGGTAAAA | |
| | | AATTGATCCTTTATTAGAGATTAAAAGATTAAGTTACTTT | |
| | | AGGGATAACAGCGTAAT | |
| AY622 | Megokris | GGGGGACGATAAGACCCTATAAAGCTTTACAATTATTTAA | 873 |
| 214 | granulosus | TTAGATTATAAACTGTTAGTATAAACTTAATTTAATTAAT | |
| | | ACTTGTTTCGTTGGGGCGACGAGAATATAATTTTGGTAAC | |
| | | TGTTCTTTTGTAAATTATTACAATAAATTTTTGGAGAAAA | |
| | | AATGATCCTTTATTAGAGATTAAAAGATTAAGTTACTTTA | |
| | | GGGATAACAGCGTAAT | |
| AY622 | Parapenaeus | GGGGGACGATAAGACCCTATAAAGCTTTACAATTATTTAA | 874 |
| 210 | politus | TTAGATTATAAATTGTTAGTATAACTTAATTTAATTGATA | |
| | | TTTGTTTCGTTGGGGCGACGAGAATATAATTTTAAGTAAC | |
| | | TGTTCTTTTATGGTTAATTACAGATTTATTTGGGTAAAAA | |
| | | TTGATCCTTTATTAGAGATTAAAAGATTAAGTTACTTTAG | |
| | | GGATAACAGCGTAAT | |
| FR849 | Solenocera | GGGGGACGATAAGACCCTATAAAGCTTTACAATAATTTAA | 875 |
| 633 | membranacea | TTAAATTATAAATTGTTAGTATAACTTAATTTAATTAATA | |
| | | TTTGTTTCGTTGGGGCGACGGGAATAAAATAGTATAACTG | |
| | | TTCTTTTATAAAATTAAACAGATTTAATTGGATAAAGATT | |
| | | GATCCTTTAATAAAGATTAAAAGATTAAGTTACTTTAGGG | |
| | | ATAACAGCGA | |
| FJ435 | Solenocera | AGGGGACGATAAGACCCTATAAAGCTTTACAATAAGTTAA | 876 |
| 649 | koelbeli | TTAAATTATAAATTGTTAGTATAACTTGGTTTAGTTATTA | |
| | | TTTGTTTCGTTGGGGCGACGAGAATATAATTATATATAAC | |
| | | TGTTCTTTTAAGAAGAAAACAAGGTTAATTGGATTAGAAT | |
| | | TGATCCTTTAATAAAGATTAAAAGATTAAGTTACTTTAGG | |
| | | GATAACAGCGTAAT | |
| NC_03 | Alcockpenaeopsis | GGGGGACGATAAGACCCTATAAAGCTTTACAATTATTTAA | 877 |
| 8069 | hungerfordii | TTAAATTGTAAATTGTTTATTTAACATGGTTTAGTTAATG | |
| | | TTTGTTTCGTTGGGGCGACGGGAATATAATTTAGTAACTG | |
| | | TTCTTTATGAGAGACAATAATTTTTGGTATTATGATCCTC | |
| | | TATTAGAGATTAAAAGATTAAGTTACTTTAGGGATAACAG | |
| | | CGTAAT | |
| FJ435 | Batepenaeopsis | GGGTGACGATAAGACCCTATAAAACTTTACAATTATTTAA | 878 |
| 641 | tenella | TTGAATGATGAATTGTTAGTATAAATTTGATTCAATTAAT | |
| | | GTTTGTTTCGTTGGGGCGACGAAAATATAATTTTATAACT | |
| | | GTTTTATAATTTTGTAACAATGATTTTTGGTGAAGATGAT | |
| | | CCTTTATTAGAGATTAAAAGATTAAGTTACTTTAGGGATA | |
| | | ACAGCGTAAT | |
| EU548 | Pandalus | ATAAAATTTTACAAGATGGGGATTTTTTTTTGAATTAAGG | 879 |
| 178 | platyceros | TTTAAATTCTATAAGTTCCTGTTTGTTGTGTTGGGGCGGC | |
| | | AAAGATAAAAATTAGTAACTGTCATTTAATTAGAATAATT | |
| | | ATAATTAGTTTAGTTGATCCTTTATTAGAGATTAAAAG | |
| FJ435 | Metapenaeus | GGGGGACGATAAGACCCTATAAAGCTTTACAGTGTATTGA | 880 |
| 639 | moyebi | TTAAATTATAAACTGTTAGTATAAACTTAATTTAGTTAAT | |
| | | AACTGTTTCGTTGGGGCGACGAGAATATAATAAACAGTAA | |
| | | CTGTTCTTTTTTTATAATAACAATAAATATTTGGTATTAA | |
| | | TTGATCCTTTATTAAAGATTAAAAGATTAAGTTACTTTAG | |
| | | GGATAACAGCGTAAT | |
| NC_04 | Metapenaeus | GGGGGACGATAAGACCCTATAAAGCTTTACAGTGTGTTAA | 881 |
| 2173 | joyneri | TTAAATTTTAAACTGTTAGTATTAACTTAATTTATTTAAT | |
| | | AACTGTTTCGTTGGGGCGACGAAAATATAATGAATAGTAA | |
| | | CTGTTTTTCTTTGTAAAATAACAATAAATTTTTGGAAGTA | |
| | | ATTGATCCTTTTTTAAAGATTAAAAGATTAAGTTACTTTA | |
| | | GGGATAACAGCGTAAT | |
| EU868 | Pandalus | GGGGGACGATAAGACCCTATAAAATTTTACAAAATCGGGG | 882 |
| 698 | montagui | TTTTTTTCTGAATTAAGGTGTAAATTTTATAAATCCCTGT | |
| | | TTGTTGTGTTGGGGCGGCAAAGATAAAAATTAGTAACTGT | |
| | | CATTTAATTAGAATAATTATAATTAGTTTAATTGATCCTT | |
| | | TATTAGAGATTAAAAGATTAAGTTACTTTAGGGATAACAG | |
| | | CGTAAT | |
| HQ241 | Heterocarpus | GGGGGACGATAAGACCCTATAAAACTTTACAAGCGAAGAT | 883 |
| 514 | parvispin | TTGTATTTTTTAAATTAAAGTATAACGTTATGTACTGTTC | |
| | a | TGTTTGTTATGTTGGGGCGACAAAGATATAATAAGTAACT | |
| | | GTTAATTTTATTGAATAGTGATAATTAGTTTAATTGATCC | |
| | | TCTCTTAGAGATTACAAGATTAAGTTACTTTAGGGATAAC | |
| | | AGCGTAAT | |
| HM014 | Penaeus | AGGGGACGATAAGACCCTATAAAGCTTGACAATAAATCGT | 884 |
| 402 | brasiliensis | TTATATTATAAACTGTTAGTGTAACTTGATTTTAAACGGT | |
| | | GTTTGTTGCGTTGGGGCGACGGGAATATAAAAGGTAACTG | |
| | | TTCTTATAACATTTAATAACAGAAATATCTGAAAAATTAA | |
| | | TGATCCTTTTTTAAAGATCATAAGATTAAGTTACTTTAGG | |
| | | GATAACAGCGTAAT | |
| GU972 | Aristeus | GGGGGACGATAAGACCCTATAAAGCTTTACAATAACCAGA | 885 |
| 651 | antennatus | ATGCATTAAGATTTGTTAGTATAATTTGGTCTATTTCGGG | |
| | | TTTGTTTCGATGGGGCGACGAGAATATAAGTTAGATAACT | |
| | | GTTCTTTTTAGGTATACAATATTAATTGGTTAAAAATTGA | |
| | | TCCTTTATTAAAGATTATAAGATTAAGTTACTTTAGGGAT | |
| | | AACAGCGTAAT | |
| GQ302 | Heterocarpus | GGGGGACGATAAGACCCTATAAAACTTTACAGGTAAATCT | 886 |
| 761 | laevigatus | GTGCTTCTTAAATTAAAGTTTAACATTGGTCATCGTCTTG | |
| | | CCTGTTGTGTTGGGGCGACAAAGATATAATTTAAATAACT | |
| | | GTCAATTTTATTAAATAGCGATAATTAGATTAGTTGATCC | |
| | | TTTATTAGAGATTATAAGATTAAGTTACTTTAGGGATAAC | |
| | | AGCGTAAT | |
| GQ302 | Heterocarpus | GGGGGACGATAAGACCCTGTAAAACTTTACAGACATTGTT | 887 |
| 759 | lepidus | TGTATTTTTGAATTAAGGTTTAATATCAATTGTTATTGTT | |
| | | GTTTGTTATGTTGGGGCGACAAAGATATAATTAGAATAAC | |
| | | TGTCAATTTTATTAAATAATGATAATTAGTTAATTGATCC | |
| | | TTTATTAGAGATTATAAGATTAAGTTACTTTAGGGATAAC | |
| | | AGCGTAAT | |
| GQ302 | Heterocarpus | GGGGGACGATAAGACCCTATAAAACTTTACAGACATTGTT | 888 |
| 745 | gibbosus | TGTATTTTTGAATTAAAGTTTAATATCAATTGTTATTGTT | |
| | | GTTTGTTATGTTGGGGCGACAAAGATATAATTGGAATAAC | |
| | | TGTCAATTTTATTAAATAGTAATAATTAGTTAATTGATCC | |
| | | TTTATTAGAGATTATAAGATTAAGTTACTTTAGGGATAAC | |
| | | AGCGTAAT | |
| JX403 | Funchalia | AGGGGACGATAAGACCCTATAAAACTTGACAATAATTTAA | 889 |
| 852 | villosa | TTAAATTATAAATTGTAAGTATAACTTGATTTAATTGATA | |
| | | CTTGTTCCGTTGGGGCGACGAGAATATAAAATAACTGTTC | |
| | | TTTTAAATATTTACATATATATTTGGTAAAGATTGATCCT | |
| | | TTATTAAAGATTAAAAGATTAAGTTACTTTAGGGATAACA | |
| | | GCGTAAT | |
| JX403 | Hemipenaeus | GGGGGACGATAAGACCCTATAAAGCTTTACAATAATCTAA | 890 |
| 847 | carpenter | ATATTCCAAGATTTGTTAGTCTAATTTGGGTTATTTAGAG | |
| | | TTTGTTTCGTTGGGGCGACGAGAATATAAATTAAATAACT | |
| | | GTTCTTTTCAGTACTACAATATTAATTGATTAAAAATTGA | |
| | | TCCTTTATTAAAGATTAAAAGATTAAGTTACTTTAGGGAT | |
| | | AACAGCGTAAT | |
| JX403 | Mesopenaeus | AGGGGACGATAAGACCCTATAAAGCTTTACAATAAAATTA | 891 |
| 849 | tropicalis | TTAAATTATAAATTGTTAGTCTAACTTAGTTTAATAATTG | |
| | | TTTGTTTCGTTGGGGCGACGAGAATATAATAATATAACTG | |
| | | TTCTTTTAAAAATTATTTAACAGATTTAATTGGATTATAA | |
| | | TTGATCCTTTAATAAAGATTAAAAGATTAAGTTACTTTAG | |
| | | GGATAACAGCGTAAT | |
| JF899 | Pelagope | AGGGGACGATAAGACCCTATAAAACTTAACAATATTTTAA | 892 |
| 809 | naeus | CTAAATTATAAATTGTAAACGTAACTTAATTTTAGTTAAT | |
| | balboae | ACTTGTTTCGTTGGGGCGACGAGAATATAAATAATAACTG | |
| | | TTCTTTTAAATATAATTCACATATATGTATGGTAAAAATT | |
| | | GATCCTTTATTAAAGATTAAAAGATTAAGTTACTTTAGGG | |
| | | ATAACAGCGTAAT | |
| JF899 | Penaeus | GGGGGACGATAAGACCCTATAAAGCTTGACAATAACTTAG | 893 |
| 806 | hathor | TTAAATTATAAATTGTTAGTATAACTTGATTTTAGCCAGG | |
| | | GTTTGTTTCGTTGGGGCGACGAGAATATAATGAATAACTG | |
| | | TTCTTTTAAATATAATTACAAAGATAATTGGTGAGCGATT | |
| | | GATCCTCTAATAGAGATTAAAAGATAAAGTTACTTTAGGG | |
| | | ATAACAGCGTAAT | |
| JF899 | Metapenaeopsis | AAGGGACGATAAGACCCTATAAAGCTTTACAATTATTTAA | 894 |
| 805 | provocatoria | TTAGATTATAAATTGTTGGTGTAACTTGATTTAATTAATA | |
| | | TTTGTTTCGTTGGGGCGACGGGAATAAAATTGAAATAACT | |
| | | GTTCTTTTATTAGTAGTCACAGATTTGTTTGATTAAAAAT | |
| | | TGATCCTTTATTAGAGATTAAAAGATTAAGTTACTTTAGG | |
| | | GATAACAGCGTAAT | |
| NC_03 | Aristeus | GGGGGACGATAAGACCCTATAAAGCTTTACAATAACCAAG | 895 |
| 9154 | virilis | ATGCACTAAGATTTGTTAGTATAATTTGGTCTATTTGGGG | |
| | | TTTGTTTCGTTGGGGCGACGAGAATATAAATTAAATAACT | |
| | | GTTCTTTTTAGATATACAATATTAATTGGTTAAAAATTGA | |
| | | TCCTTTATTAAAGATTACAAGATTAAGTTACTTTAGGGAT | |
| | | AACAGCGTAAT | |
| KJ486 | Aristeus | GGGGGACGATAAGACCCTATAAAGCTTTACAATAACTGAA | 896 |
| 492 | alcock | ATACATTAAGATTTGTTAGTATAATTTGGTTTATTTTGGG | |
| | | TTTGTTTCGTTGGGGCGACGAGAATATAAGTTAGATAACT | |
| | | GTTCTT | |
| KF983 | Penaeus | AGGGGACGATAAGACCCTATAAAGCTTGACAATAAGTCAT | 897 |
| 532 | aztecus | TTATATTATAAATTATTAGTATAACTTGATATTAAGTGGT | |
| | | GTTTGTTGCGTTGGGGCGACGAGAATATAAAAGGTAACTG | |
| | | TTCTTATAACATTTAATAACAGAAATATCTGGAAATTTAA | |
| | | TGATCCTTTTTTAAAGATCATAAGATTAAGTTACTTTAGG | |
| | | GATAACAGCGTAAT | |
| KF023 | Heterocarpus | GGGGGACGATAAGACCCTATAAAACTTTACAGACATTGTT | 898 |
| 186 | abulbus | TGTATTTTTGAATTAAGGTTTAATATCAATTGTTATTGTT | |
| | | GTTTGTTATGTTGGGGCGACAAAGATATAATTGGAATAAC | |
| | | TGTCAATTTTATTAAATAGTAATAATTAGTTAATTGATCC | |
| | | TTTATTAGAGATTATAAGATTAAGTTACTTTAGGGATAAC | |
| | | AGCGTAAT | |
| JX403 | Penaeussetiferus | AGGGGACGATAAGACCCTATAAAGCTTTACAATAAATTAC | 899 |
| 862 | | TTATATTATAAACTGTTAGTATAACTTGATTTTAGGTAAT | |
| | | ATTTGTTGCGTTGGGGCGACGAGAATATAATGAGTAACTG | |
| | | TTCTTAAGTTAATTAATAACATAAATATATGGAAGATGAA | |
| | | TGATCCTCTTTTAGAGATCATAAGATTAAGTTACTTTAGG | |
| | | GATAACAGCGTAAT | |
| JX403 | Cerataspis | GGGGGACGATAAGACCCTATAAAGCTTTACAATAACTCAG | 900 |
| 860 | monstrosus | GTGTACTAAGATTTGTTTGTATAATTTGGTTTACCTGAGA | |
| | | CTTGTTTCGTTGGGGCGACGAGAATATAATTAAATAACTG | |
| | | TTCTTTTGGAGTTTACAGTATTAATTGGTTAAAAATTGAT | |
| | | CCTTTATTAAAGATTAAAAGATTAAGTTACTTTAGGGATA | |
| | | ACAGCGTAAT | |
| JX403 | Pleoticus | GGGGGACGATAAGACCCTATAAAGCTTTATAATAATTTAA | 901 |
| 857 | robustus | TTAAATTATAAATTGTTAGTATAACTTAATTTAATTAGTA | |
| | | TTTATTTCGTTGGGGCGACGAGAATATAATAATATAACTG | |
| | | TTCTTTTATAGATTAAACAAATTTAATTGGATAAAAATTG | |
| | | ATCCTTTTATAAAGATTAAAAGATTAAGTTACTTTAGGGA | |
| | | TAACAGCGTAAT | |
| JX403 | Aristaeopsis | GGGGGACGATAAGACCCTATAAAGCTTTACAATAACTTGA | 902 |
| 854 | edwardsiana | ATATACTAAAATTTGTTAGTGTAACTTAATATATTTGAGA | |
| | | TTTGTTTCGTTGGGGCGACGAGAATATAAATAAATAACTG | |
| | | TTCTTTTGCTTAAATATACAATAATAATTGGTTAAATATT | |
| | | GATCCTTTATTAAAGATTAAAAGATTAAGTTACTTTAGGG | |
| | | ATAACAGCGTAAT | |
| JX403 | Solenoce | GAGGGACGATAAGACCCTATAAAGCTTTACAATAAGATAA | 903 |
| 853 | ra | TTAAATTATAAATTGTTAGTTTAACTTAATTTAATTATTA | |
| | necopina | TTTGTTTCGTTGGGGCGACGAGAATATAATAGTATAACTG | |
| | | TTCTTTTGCAATTTAAACAAAATTAATTGGATAAAAATTG | |
| | | ATCCTTTAATAAAGATTAAAAGATTAAGTTACTTTAGGGA | |
| | | TAACAGCGTAAT | |
| KP059 | Parapenaeus | GGGGGACGATAAGACCCTATAAAGCTTTACAATTATTTAA | 904 |
| 272 | indicus | TTAAATTATAAATTGTTAGTATAACTTAATTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 905 |
| 270 | cayrei | TTAAATTATAAATTGTTAGTATAACTTAACTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 906 |
| 273 | fissurus | TTAAATTATAAATTGTTAGTATAACTTAGTTTAGTTGATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 907 |
| 274 | investigatoris | TTGGATTATAAATTGTTAGTATAACTTAATTTAGTTGATA | |
| | | TTTGTTTCGTTGGGGCGACGAGAATATAATTTTTAAGTAA | |
| | | CTGTTCTTTTGTAGTTAATTACAGATTTATTTGGGTAAAA | |
| | | ATTGATCCTTTATTAGAGATTAAAAGATTAAGTTACTTTA | |
| | | GGGATAACAGCGTAAT | |
| KP059 | Parapenaeus | GGGGGACGATAAGACCCTATAAAGCTTTACAATTATTTAA | 908 |
| 271 | fissuroides | TTAAATTATAAATTGTTAGTATAACTTAATTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 909 |
| 269 | australiensis | TTAAATTATAAATTGTTAGTATAACTTAATTTAGTTGATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 910 |
| 267 | americanus | TTAAATTATAAATTGTTAGTATAACTTAATTTAATTAATA | |
| | | TTTGTTTCGTTGGGGCGACGAGAATATAATTTTAAGTAAC | |
| | | TGTTCTTTTGTGGTTAATTACAGATTTATTTGGGTAAAAA | |
| | | TTGATCCTTTATTAGAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 911 |
| 268 | ruberoculatus | TTAAATTATAAATTGTTAGTATAACTTAATTTAGTTGATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| NC_04 | Heterocarpus | GGGGGACGATAAGACCCTATAAAACTTTACAAGCGAAGAT | 912 |
| 0855 | ensifer | TTGTATTTTTTAAATTAAAGTATAACGTTATGTACTGTTC | |
| | | TGTTTGTTATGTTGGGGCGACAAAGATATAATAAGTAACT | |
| | | GTTAATTTTATTGAATAGTGATAATTAGTTTAATTGATCC | |
| | | TCTCTTAGAGATTACAAGATTAAGTTACTTTAGGGATAAC | |
| | | AGCGTAAT | |
| KP059 | Kishinouyepenaeopsis | GGGGGACGATAAGACCCTATAAAGCTTTACAATTGTTTAA | 913 |
| 284 | cornuta | TTAGATTGTAAATTGTTTATTTAACATAATTTAATTAATG | |
| | | CTTGTTTCGTTGGGGCGACGGAAATATAATTAGTAACTGT | |
| | | TTTTTATAAAAAACAATTATTTTTGGTAAAATGATCCTCT | |
| | | ATTAGAGATTAAAAGATCAAGTTACTTTAGGGATAACAGC | |
| | | GTAAT | |
| KP059 | Parapenaeus | GGGGGACGATAAGACCCTATAAAGCTTTACAATTATTTAA | 914 |
| 283 | sextuberculatus | TTAAATTATAAATTGTTAGTATAACTTAATTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 915 |
| 281 | perezfarf | TTAGATTATAAATTGTTAGTATAACTTAATTTAGTTAATA | |
| | antae | TTTGTTTCGTTGGGGCGACGAGAATATAATTTTAAGTAAC | |
| | | TGTTCTTTTATAGTTAATTACAAATTTATTTGGGTAAAAA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 916 |
| 280 | murrayi | TTGGATTATAAATTGTTAGTATAACTTAGTTTAGTTGATA | |
| | | TTTGTTTCGTTGGGGCGACGAGAATATAATTTTTAAGTAA | |
| | | CTGTTCTTTTGTAGTTAATTACAGATTTATTTGGGTAAAA | |
| | | TTGATCCTTTATTAGAGATTAAAAGATTAAGTTACTTTAG | |
| | | GGATAACAGCGTAAT | |
| KP059 | Parapenaeus | AGGGGACGATAAGACCCTATAAAGCTTTACAATTGTTTAG | 917 |
| 278 | longipes | TTGGATTATAAATTGTTAGTATAACTTAATTTAGTTAGTA | |
| | | TTTGTTTCGTTGGGGCGACGAGAATATAATTTAAGATAAC | |
| | | TGTTCTTTTTAATTAATTACAAATTTGTTTGGGAGAAGAT | |
| | | TGATCCTTTATTAAAGATTATAAGATTAAGTTACTTTAGG | |
| | | GATAACAGCGTAAT | |
| KP059 | Parapenaeus | GGGGGACGATAAGACCCTATAAAGCTTTACAATTATTTAA | 918 |
| 276 | lanceolatus | TTAAATTATAAATTGTTAGTATAACTTAATTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KP059 | Parapenaeus | GGGGGACGATAAGACCCTATAAAGCTTTACAATTATTTAA | 919 |
| 275 | kensleyi | TTAAATTATAAATTGTTAGTATAACTTAATTTAGTTAATA | |
| | | TTTGTTTCGTTGGGGCGACGAGAATATAATTTTAAGTAAC | |
| | | TGTTCTTTTATAGTTAATTACAAATTTATTTGGGTAAAGA | |
| | | TTGATCCTTTATTATAGATTAAAAGATTAAGTTACTTTAG | |
| | | GGATAACAGCGTAAT | |
| KT372 | Heterocarpus | GGGGGACGATAAGACCCTATAAAACTTTACAGACATTGTT | 920 |
| 710 | chani | TGTATTTTTGAATTAAGGTTTAATATCAATTGTTATTGTT | |
| | | GTTTGTTATGTTGGGGCGACAAAGATATAATTGGAATAAC | |
| | | TGTCAATTTTATTAAATAGTGATAATTAGTTAATTGATCC | |
| | | TTTATTAGAGATTATAAGATTAAGTTACTTTAGGGATAAC | |
| | | AGCGTAAT | |
| KP725 | Heterocarpus | AGGGGACGATAAGACCCTATAAAACTTTACAGATATAATT | 921 |
| 531 | woodmasoni | ATACTTATTAAAATAAAGTTTAATAGTATTTATTATTTTA | |
| | | TCTGTTATGTTGGGGCGACAAAGATATAATAAAGTAACTG | |
| | | TTAGTTTTATTTGATAATAATAATTAGTTTAATTGATCCT | |
| | | TTATTAAAGATTATAAGAATAAGTTACTTTAGGGATAACA | |
| | | GCGTAAT | |
| KP725 | Heterocarpus | GGGGGACGATAAGACCCTATAAAACTTTACAAGCAAAGAT | 922 |
| 530 | sibogae | TTGTATTTTTTAAATTAAAGTATAACGTTATGTACTGTTC | |
| | | TGTTTGTTATGTTGGGGCGACAAAGATATAATAAGTAACT | |
| | | GTTAATTTTATTGAATAATGATAATTAGTTTAATTGATCC | |
| | | TCTCTTAGAGATTACAAGATTAAGTTACTTTAGGGATAAC | |
| | | AGCGTAAT | |
| KP725 | Heterocarpus | GAGGGACGATAAGACCCTATAAAACTTTACAAATATATTT | 923 |
| 528 | dorsalis | GTAATTTTTAAATTTTAGTTTAACATCAATTACTGGTTTG | |
| | | TTTGTTGTGTTGGGGCGACAATGATATAATTAAATAACTG | |
| | | TCAATTTTATTAAATAGTGATAATTAGTTTAATTGATCCT | |
| | | TTACTAGAGATTATAAGATTAAGTTACTTTAGGGATAACA | |
| | | GCGTAAT | |
| KP721 | Metapenaeopsis | GAGGGACGATAAGACCCTATAAAGCTTTACAATTATTTAA | 924 |
| 230 | andamanensis | TTAGATTATAAATTGTTGGTATAACTTGATTTAATTAATA | |
| | | TTTGTTTCGTTGGGGCGACGAGAATAAAATTAAAATAACT | |
| | | GTTCTTTTATTAGTAGTTACAAATTTGTTTGGTTAAAAAT | |
| | | TGATCCTTTATTAGAGATCAAAAGATTAAGTTACTTTAGG | |
| | | GATAACAGCGTAAT | |
| KP725 | Heterocarpus | GGGGGACGATAAGACCCTATAAAACTTTACAGACATTGTT | 925 |
| 527 | corona | TGTATTTTTGAATTAAAGTTTAATATCAATTGTTATTGTT | |
| | | GTTTGTTATGTTGGGGCGACAAAGATATAATTGGAATAAC | |
| | | TGTCAATTTTATTAAATAGTAATAATTAGTTAATTGATCC | |
| | | TTTATTAGAGATTATAAGATTAAGTTACTTTAGGGATAAC | |
| | | AGCGTAAT | |
| KP721 | Metapenaeopsis | GAGGGACGATAAGACCCTATAAAGCTTTACAATTATTTAA | 926 |
| 226 | coniger | TTAGATTATAAATTGTTGGTATAACTTGATTTAATTAATA | |
| | | TTTGTTTCGTTGGGGCGACGAGAATAAAATTAAAATAACT | |
| | | GTTCTTTTATTAGTAGTTACAGATTTGTTTGGTTAAAAAT | |
| | | TGATCCTTTATTAGAGATCAAAAGATTAAGTTACTTTAGG | |
| | | GATAACAGCGTAAT | |
| KP081 | Macrobrachium | AATGGGACGATAAGACCCTATAAAACTTAATATAATTTTG | 927 |
| 666 | idella | GCTTAATTTGTGATTATTAGTGAAAAGTAGTTTTGCCGGG | |
| | | TTTATATTTCGTTGGGGAGATGTAGATATAATATGTAACT | |
| | | GTCTATATAATTTAATAATTATAATTAGTATTTGATCCTT | |
| | | CTTTGTGGATTAGAAGAATAAGTTACTTTAGGGATAACAG | |
| | | CGTGAT | |
| KX162 | Trachysal | GAGGGACGATAAGACCCTATAAAGCTTTATAACTAGCTGA | 928 |
| 742 | ambria | TTAAATTATAAATTATTAGTATAAACTTAGTTTAATTGGT | |
| | brevisutur | GCTTATTTTGTTGGGGCGACAAGAACATAAAATTGTAACT | |
| | ae | GTTCTTTTATAGTTTATATCATTAATGTTTGGTAAATAAT | |
| | | TGATCCTTTGTTAAAGATTAAAAGATTAAGTTACTTTAGG | |
| | | GATAACAGCGTAAT | |
| KX162 | Trachysal | GGGGGACGATAAGACCCTATAAAGCTTTACAATTATCTAG | 929 |
| 734 | ambria | TTGAATTATAAATTATTAGTATAAACTTAGTTTAATTAGT | |
| | aspera | GTTTGTTTCGTTGGGGCGACGAGAATATAATTTTGTAACT | |
| | | GTTCTTTTATGATTTGTTACAATGATTTTTGGAAAAAATT | |
| | | GATCCTTTATTAAAGATTAAAAGATTAAGTTACTTTAGGG | |
| | | ATAACAGCGTAAT | |
| KX162 | Trachysal | GGGGGACGATAAGACCCTATAAAGCTTTACAATTATTTGA | 930 |
| 733 | ambria | TTAACTTATAAATTATTAGTATAAACTTAACTTAATCAAT | |
| | albicoma | GTTTGTTTCGTTGGGGCGACGAGAATATAATTTTGTAACT | |
| | | GTTCTTTAGTAGTTTGTTACAATAATTTTTGGTAAAAAAT | |
| | | TGATCCTTTATTAAAGATTAAAAGATTAAGTTACTTTAGG | |
| | | GATAACAGCGTAAT | |
| NC_04 | Euphausia | AAGTGACGATAAGACCCTATAAAACTTGATAAATAAAATA | 931 |
| 0987 | superba | AAAAAAGTGGAGTAGTTGATTAAAACTTTTATTGTGGAAT | |
| | | TTATTTTATTGGGGCGATAAAAATATAAAAAGTAACTGTT | |
| | | TTTAAAGTTTTAATGATTTATAATCAGATTGTGTGAATGA | |
| | | TCCTTTAATAAAGATTATAAGATTAAGTTACTTTAGGGAT | |
| | | AACAGCGTAAT | |
| KU133 | Solenocera | GGGGGACGATAAGACCCTATAAAGCTTTACAATAAATTAA | 932 |
| 278 | hextii | TTAAATTATAAATTATTAGTTTAACTTAATTTAATTAGTG | |
| | | TTTGTTTCGTTGGGGCGACGAGAATATAAATTATATAACT | |
| | | GTTCTTTTAGGTGTATAACAAGATTAATTGGAGAAATATT | |
| | | GATCCTTTAGTAAAGATTAAAAGATTAAGTTACTTTAGGG | |
| | | ATAACAGCGTAAT | |
| KU133 | Hymenopenaeus | AAGGGACGATAAGACCCTATAAAGCTTTACAATAAGTTAA | 933 |
| 288 | equalis | TTAAATTATAAATTGTTAGTATAACTTAATTTAATTAATG | |
| | | TTTGTTTCGTTGGGGCGACGGGAATATAATTATATAACTG | |
| | | TTCTTTTATAAATTTAACAAAATTAATTGGATAATAATTG | |
| | | ATCCTTTAATAAAGATTAAAAGATTAAGTTACTTTAGGGA | |
| | | TAACAGCGTAAT | |
| KT959 | Rimapenaeus | AGGGGACGATAAGACCCTATAAAGCTTTACAATTATCTAA | 934 |
| 496 | constrictus | TTAAATTATAAATTGTTAGTTTAATCTTAATTTAATTAGT | |
| | | GTTTGTTTCGTTGGGGCGACGAGAATATAATGTTTTGTAA | |
| | | CTGTTCTTTTGTAATTTGTTACAGTTAATAGTTGGTAAAA | |
| | | GATTGATCCTTTATTAAAGATTAAAAGATTAAGTTACTTT | |
| | | AGGGATAACAGCGTAAT | |
| KT952 | Crangon | AATGGGAAGACAAGACCCTATAAAACTTTACAAAATATAT | 935 |
| 497 | crangon | TAATAAATAATTTGGTAGTTTAAAATTATATTAGGTTATT | |
| | | TTTGTTGAGCTGGGGCGGTTTTTATAAAAAAATTAACTGT | |
| | | AAGTTAAATTAAAATATAAATATATAGTTTAATAAGATCC | |
| | | TTAGGTTTGGAGATTAAAAGACTAAGTTACTTTAGGGATA | |
| | | ACAGCATAATTT | |
| KX162 | Trachypenaeus | GAGGGACGATAAGACCCTATAAAGCTTTACAATTATTTAA | 936 |
| 771 | anchoralis | TTAGATTATAAATTGTTAGTATAAACTTAGTTTAATTAAT | |
| | | GCTTGTTTCGTTGGGGCGACGAGAATATAATTTTGGTAAC | |
| | | TGTTCTTTTGTAAGCTATTACAATAAATTTTTGGAGAAAA | |
| | | AATGATCCTTTATTAAAGATTAAAAGATTAAGTTACTTTA | |
| | | GGGATAACAGCGTAAT | |
| KX162 | Megokris | GGGGGACGATAAGACCCTATAAAGCTTTACAATTATTTAA | 937 |
| 770 | pescadoreensis | TTAGATTATAAACTGTTAGTATAAACTTAATTTAATTAAT | |
| | | ACTTGTTTCGTTGGGGCGACGAGAATATAATTTTGGTAAC | |
| | | TGTTCTTTTGTAAATTATTACAATAAATTTTTGGAGAAAA | |
| | | AATGATCCTTTATTAGAGATTAAAAGATTAAGTTACTTTA | |
| | | GGGATAACAGCGTAAT | |
| KX162 | Trachysal | GAGGGACGATAAGACCCTATAAAGCTTTACAATTATTTAA | 938 |
| 768 | ambria | CTAAATTATGAACTGTTAGTATAAATTTAATTTAATTAAT | |
| | longipes | GTTTGTTTCGTTGGGGCGACGAGAATATAATTTTTGTAAC | |
| | | TGTTCTTTTGTAGTTTGTTGCAATAGTTTTTGGAAAAAGA | |
| | | TTGATCCTTTATTAAAGATTAAAAGATTAAGTTACTTTAG | |
| | | GGATAACAGCGTAAT | |
| KX162 | Trachysal | GGGGGACGATAAGACCCTATAAAGCTTTACAATTATTTGG | 939 |
| 764 | ambriastaro | TTAAATTATAAATTGTTAGTATAAACTTAATTTAATTGAT | |
| | bogatovi | GTTTGTTTCGTTGGGGCGACGAGAATATAATTTAGTAACT | |
| | | GTTCTTTTATAATTTGTTACAATGATTTTTGGAAAAGAAA | |
| | | TGATCCTCTATTAGAGATTAAAAGATTAAGTTACTTTAGG | |
| | | GATAACAGCGTAAT | |
| KX162 | Trachysal | GGGGGACGATAAGACCCTATAAAGCTTTACAATTATTTGG | 940 |
| 758 | ambria | TTAAATTATGAATTGTTAGTATAAATTTAATTTAATTGAT | |
| | nansei | GTTTGTTTCGTTGGGGCGACGGGAATATAATTTTGTAACT | |
| | | GTTCTTTTATAATTTGTTACAATAATTTTTGGTAAAAGAT | |
| | | TGATCCTTTATTAAAGATTAAAAGATTAAGTTACTTTAGG | |
| | | GATAACAGCGTAAT | |
| KX162 | Trachysal | GAGGGACGATAAGACCCTATAAAGCTTTACAATTATTTAA | 941 |
| 757 | ambria | TTAGATTATAAATTGTTAGTATAAACTTAATTTAGTTAGT | |
| | malaiana | GTTTGTTTCGTTGGGGCGACGAGAATATAATTTTGGTAAC | |
| | | TGTTCTTTTGTAAATTATTACAATAAATTTTTGGAGAAAA | |
| | | AATGATCCTTTATTAGAGATTAAAAGATTAAGTTACTTTA | |
| | | GGGATAACAGCGTAAT | |
| KX162 | Trachysal | GGGGGACGATAAGACCCTATAAAGCTTTACAATTATTTAG | 942 |
| 755 | ambria | TTAAATTATAAATTGTTAGTATAAACTTAATTTAATTGAT | |
| | dentata | GTTTGTTTCGTTGGGGCGACGAGAATATAATTTTGTAACT | |
| | | GTTCTTTTATAGTTTGTTACAATTATTTTTGGTAAAAGAT | |
| | | TGATCCTTTATTAAAGATTAAAAGATTAAGTTACTTTAGG | |
| | | GATAACAGCGTAAT | |
| KX162 | Trachysal | GGGGGACGATAAGACCCTATAAAGCTTTACAATTATTTAG | 943 |
| 749 | ambria | TTGGATTATAAATTGTTAGTATAAACTTAATTTAACTGGT | |
| | parvispina | GTTTGTTTCGTTGGGGCGACGGGAATATAATTTTGTAACT | |
| | | GTTCTTTTATAACTTGTTACAATTATTTTTGGTAAAAATT | |
| | | GATCCTTTATTAAAGATTAAAAGATTAAGTTACTTTAGGG | |
| | | ATAACAGCGTAAT | |
| LC464 | Crangon | ACTTTACATAATATATTAATAAATAATTTGGTAGTTTAAA | 944 |
| 546 | uritai | ATTATGTTAGGTTATTTATGTTGAGCTGGGGCGGTTTTTA | |
| | | TAAAAAAAATAACTGTAAGTTAAATTAAAATATAAATATA | |
| | | TAGTTTAATAAGATCCTTAAATTTGGAGATTGGAAGACTA | |
| | | AGTT | |
| LC341 | Pandalus | GGGGGACGATAAGACCCTATAAAATTTTACAAAATTGGGA | 945 |
| 266 | borealis | TTTTTTTTTGAATTAAGGTTTAAATTCTATAAGTTCTTAT | |
| | | TTGTTTTGTTGGGGCGGCAAAGATAAAAATTAGTAACTGT | |
| | | CATTTAATTAGAATAATTATAATTAGTTTAATTGATCCTT | |
| | | TATTAGAGATTAAAAGATTAAGTTACTTTAGGGATAACAG | |
| | | CGTAAT | |
| LC150 | Metapenaeus | GGGGGACGATAAGACCCTATAAAGCTTTACGGTTTGTTAA | 946 |
| 203 | monoceros | TTAAATTATAAATTGTTAGTATAAACTTAATTTAGCTAAC | |
| | | AGCTGTTTCGTTGGGGCGACGAAAATATAATAGATAGTAA | |
| | | CTGTTTTTCTTTTATAATAACAATGATTTTTGGTAGTAAT | |
| | | TGATCCTTTATTAAAGATTAAAAGATTAAGTTACTTTAGG | |
| | | GATAACAGCGTAAT | |
| LC121 | Pandalus | GGGGGACGATAAGACCCTATAAAATTTTACAGAGTCGGGA | 947 |
| 756 | nipponensis | TCTTTTTTTAAATTAGTGTTTAAATTCTATAGGTTCTTGT | |
| | | TTGTTTTGTTGGGGCGACAAAGATAAAATTTAGTAACTGT | |
| | | CATTTAATTAGAATAAGTATAATTAGTTTAGTTGATCCTT | |
| | | TATTAGAGATTAAAAGATTAAGTTACTTTAGGGATAACAG | |
| | | CGTAAT | |
| KY419 | Hadropenaeus | GAGGGACGATAAGACCCTATAAAGCTTTACAATAAAATGG | 948 |
| 832 | lucasii | TTAAATTATAAATTATTAGTATAACTTAATTTAATTATTG | |
| | | TTTGTTTCGTTGGGGCGACGAGAATATAATAATATAACTG | |
| | | TTCTTTATGAAAATAAACAAAATTAATTGGATAAAGATTG | |
| | | ATCCTTTAATAAAGATTAAAAGATTAAGTTACTTTAGGGA | |
| | | TAACAGCGTAAT | |
| KY316 | Ganjampenaeopsis | TTAAAAAGTTTATTATACCCAGGTAATAAGCAGTTATGAT | 949 |
| 150 | uncta | CTTGAAACTTAAAGGATTTGGCGGTAATTTAGTCTAGTTA | |
| | | GAGGAACCTGTCCTGTAATCGATAAACCACGCAGTATCTT | |
| | | ACTTTATCTTGAAAATTTATCAGTTTATATACCATCATTA | |
| | | TTAGATAACTTTAAAAAGGTAGAGAAGTTATTGAAATAAT | |
| | | TTTAAGTTAGTATATTAGATCAAGGTGTAGCTAATGATAA | |
| | | AGTAGAGAT | |
| KX530 | Solenocera | GGGGGACGATAAGACCCTATAAAGCTTTACAATAATTTAA | 950 |
| 803 | annectens | TTAAATTATAAATTATTAGTTTAACTTAATTTAATTAATA | |
| | | ATTGTTTCGTTGGGGCGACGAGAATATAAATTATATAACT | |
| | | GTTCTTTTAAAAATTTAACAAATTTAATTGGATGAAAATT | |
| | | GATCCTTTAATAAAGATTAAAAGATTAAGTTACTTTAGGG | |
| | | ATAACAGCGTAAT | |
| KX574 | Solenocera | AGGGGACGATAAGACCCTATAAAGCTTTACAATAAGTTAA | 951 |
| 332 | melantho | TTAAATTATAAATTATTAGTTTAACTTAATTTAATTAGTG | |
| | | TTTGTTTCGTTGGGGCGACGAGAATATAAATTATATAACT | |
| | | GTTCTTTTAAAGTAAATAACAAGATTAATTGGAGGAAGAT | |
| | | TGATCCTTTAATAAAGATTAAAAGATTAAGTTACTTTAGG | |
| | | GATAACAGCGTAAT | |
| MH045 | parapenaeopsis | TTAGAGGGACGATAAGACCCTATAAAGCTTTACAATTATT | 952 |
| 067 | stylifera | AAATTGAGCTGTAAATTGTTTGTTATAACGTGGCTAAGTT | |
| | | AATGTTTGTTTCGTTGGGGCGACGGGAATATAATTAGTAA | |
| | | CTGTTCTTATATATATATATATAACAATAATTTTTGGAAA | |
| | | AATGATCCTCTAATAGAGATTAAAAGATTAAGTTACTTTA | |
| | | GGGATAACAGCGTAAT | |
| MG77 | Penaeus | GGGGGACGATAAGACCCTATAAAGCTTGACAATAATTTCG | 953 |
| 2559 | japonicus | TTATATTATAAATTGTTAGTATAACTTGATTTTAACGGGA | |
| | | GTTTGTTTCGTTGGGGCGACGGGAATATAATAAATAACTG | |
| | | TTCTTTTAAATATAATTACAAAAATACTTGGTAAATAATT | |
| | | GATCCTCTATTAGAGATTAAAAGATTAAGTTACTTTAGGG | |
| | | ATAACAGCGTAAT | |
| MG00 | Penaeus | AGGGGACGATAAGACCCTATAAAGCTTGACAATAAGTTGT | 954 |
| 1109 | brevirostris | TTATATTATAAATTGTTAGTATAACTTGATTTTAAGCGGT | |
| | | GTTTGTTACGTTGGGGCGACGAGAATATAAAAGGTAACTG | |
| | | TTCTTATATTGTTTAGTGACAGATATATTTGGAGAGTTAA | |
| | | TGATCCTTTCTTAAAGATCACAAGATTAAGTTACTTTAGG | |
| | | GATAACAGCGTAAT | |
| MG00 | Penaeus | GGGGGACGATAAGACCCTATAAAGCTTGACAATAAGCCGT | 955 |
| 1088 | notialis | TTATATTATAAATTGTTAGTATAACTTGATTTTAAATGGT | |
| | | GTTTGTTGCGTTGGGGCGACGAGAATATAAAAGATAACTG | |
| | | TTCTTATAATATTTAATAACAGAAATAGTTGGAAAATTAA | |
| | | TGATCCTTTCTTTAAGATCATAAGATTAAGTTACTTTAGG | |
| | | GATAACAGCGTAAT | |
| MG00 | Penaeus | GGGGGACGATAAGACCCTATAAAGCTTGACAATAAGCCGT | 956 |
| 1081 | duorarum | TTATATTATAAATTGTTAGTATAACTTGATTTTAAATGGT | |
| | | GTTTGTTGCGTTGGGGCGACGAGAATATAAAAGATAACTG | |
| | | TTCTTATAATATTTAATAACAGAAATAGCTGGAAAATTAA | |
| | | TGATCCTTTCTTTAAGATCATAAGATTAAGTTACTTTAGG | |
| | | GATAACAGCGTAAT | |
| MF490 | Penaeusschmitti | AGGGGACGATAAGACCCTATAAAGCTTTACAATAAATTAC | 957 |
| 229 | | TTATATTATAAACTGTTAGTATAACTTGATTTTAGGTAAT | |
| | | ATTTGTTGCGTTGGGGCGACGAGAATATAACGAGTAACTG | |
| | | TTCTTAAGTTAATTAATAACATAGATATATGGAAGATGAA | |
| | | TGATCCTCTTTTAGAGATCATAAGATTAAGTTACTTTAGG | |
| | | GATAACAGCGTAAT | |
| MF490 | Artemesia | AAGGGACGATAAGACCCTATAAAGCTTTACAATTATTTAA | 958 |
| 228 | longinaris | TTAAATTGAAAATTATTAGTATAACTTGATTTAGTTAATA | |
| | | GTTGTTTCGTTGGGGCGACGAGAATATAATTATAAATAAC | |
| | | TGTTCTTTTTACAAATAACAAATTTGTTTGGTTAAAAATT | |
| | | GATCCTTTATTAAAGATTAAAAGATTAAGTTACTTTAGGG | |
| | | ATAACAGCGTAAT | |
| MF490 | Penaeus | AGGGGACGATAAGACCCTATAAAGCTTAACAATAAGTTGT | 959 |
| 143 | subtilis | CTATATTATAAATTGTTAGTATAACTTGATATCAGACGGC | |
| | | GTTTGTTGCGTTGGGGCGACGAGAATATAAGAGGTAACTG | |
| | | TTTTTAGATTATTTAATAACAGAAATATCTGGAAATTTAA | |
| | | TGATCCTTTTTTAGAGATCATAAGATTAAGTTACTTTAGG | |
| | | GATAACAGCGTAAT | |
| NC_01 | Penaeus | AGGGGACGATAAGACCCTATAAAGCTTTACAATAAGTTAT | 960 |
| 2060 | stylirostris | CTATATTATAAATTGTTAGTATAACTTGATTTTAGATAAT | |
| | | ATTTGTTGCGTTGGGGCGACGGGAATATAATGAGTAACTG | |
| | | TTCTTAAGCTATTTAATAACAGGAATTTCTGGAAAATTAA | |
| | | TGATCCTCTTTTAGAGATCATAAGATTAAGTTACTTTAGG | |
| | | GATAACAGCGTAAT | |
| NC_00 | Penaeus | AGGGGACGATAAGACCCTATAAAGCTTTACAATAAGTTAC | 961 |
| 9626 | vannamei | CTATATTATAAATTGTTAGTATAACTTGAGTTTAGGTAAC | |
| | | GTTTGTTGCGTTGGGGCGACGAGAATATAATAAGTAACTG | |
| | | TTCTTAAGTTATTTAATGACAGAAATTTCTGGAAAATTAA | |
| | | TGATCCTCTACTAGAGATCATAAGATTAAGTTACTTTAGG | |
| | | GATAACAGCGTAAT | |
| NC_00 | Macrobrachium | GGGGGACGATAAGACCCTATAAAACTTAATATAATTTAGG | 962 |
| 6880 | rosenbergii | CTTAACTTGCGATGTGGGTGAAAAGTAGTTTTGTCTGGTT | |
| | | TATATTTCGTTGGGGAGATGAAGATATAATGAGTAACTGT | |
| | | CTATAAAATTTTATAGCATTGACTAGAATTTGATCCTTCC | |
| | | TTGGGGATTAGGAGAATAAGTTACTTTAGGGATAACAGCG | |
| | | TGAT | |
| NC_00 | Penaeus | AGGGGACGATAAGACCCTATAAAACTTAACAATAATTTGA | 963 |
| 2184 | monodon | TTAAATTATAAATTGTTAGTATAACTTGATTTTAATTAAT | |
| | | GTTTGTTGCGTTGGGGCGACGGGAATATAATTAGTAACTG | |
| | | TTCTTAAATATTTTATTAACAAGTATAATTGAAGAATAAT | |
| | | TGATCCTTTATTAAAGATTAAAAGATTAAGTTACTTTAGG | |
| | | GATAACAGCGTAAT | |
| MK470 | Pandalus | GGGGGACGATAAGACCCTATAAAATTTTACAAAATTAGGA | 964 |
| 792 | hypsinotus | TTTTTTTTTAAATTAAGGTTTAAATTCTATAAGTTCTTGT | |
| | | TTGTTGTGTTGGGGCGGCAAAGATAAAAATTAGTAACTGT | |
| | | CATTTAATTAGAATAAGTATAATTAGTTTAGTTGATCCTT | |
| | | TATTAGAGATTAAAAGATTAAGTTACTTTAGGGATAACAG | |
| | | CGTAAT | |
| MK430 | Ganjam | GGGTGACGATAAGACCCTATAAAACTTTACAATTATTTAA | 965 |
| 861 | penaeopsis | TTGAATGATGAATTGTTAGTATAAATTTGATTCAATTAAT | |
| | uncta | GTTTGTTTCGTTGGGGCGACGAAAATATAATTTTATAACT | |
| | | GTTTTATAATTTTGTAACAATGATTTTTGGTGAGGATGAT | |
| | | CCTTTATTAGAGATTAAAAGATTAAGTTACTTTAGGGATA | |
| | | ACAGCGTAAT | |
| MK000 | Pandalus | GGGGGACGATAAGACCCTATAAAATTTTACAAAATTGGGA | 966 |
| 270 | jordani | TTTTTTTTTTGAATTAATGTTTAAATTTTATGAGTTCCTG | |
| | | TTTGTTTTGTTGGGGCGGCAAAGATAAAAATTAGTAACTG | |
| | | TCATTTAATTAGAATAATTATAATTAGTTTAATTGATCCT | |
| | | TTATTAGAGATTAAAAGATTAAGTTACTTTAGGGATAACA | |
| | | GCGTAAT | |
| NC_02 | Macrobrachium | ATGGGACGATAAGACCCTATAAAACTTGATATAAATTTGA | 967 |
| 7602 | bullatum | CTTAATTTGCGATTGTTTGTGAAAAGTAGTTTTGCTGTGT | |
| | | TTATATTTCGTTGGGGAGATGTAGATATAATATGTAACTG | |
| | | TCTGTTTAATGTAATAATTATAATTAGTTTGTGATCCTTC | |
| | | ATTTGTGGATTAGAAGAATAAGTTACTTTAGGGATAACAG | |
| | | CGTGAT | |
| NC_02 | Penaeus | AGGGGACGATAAGACCCTATAAAGCTTGACAATAATTTAA | 968 |
| 6885 | penicillatus | TTATACTATCAATTGTTAGTATAACTTGGTTTTAATTAAG | |
| | | ATTTGTTGCGTTGGGGCGACGAGAATATAATAGGTAACTG | |
| | | TTCTTAAATATTTAATAACAAATATAATTGAAAATTAGTG | |
| | | TGATCCTCTATTAGCGATTAAAAGATTAAGTTACTTTAGG | |
| | | GATAACAGCGTAAT | |
| NC_02 | Penaeus | AGGGGACGATAAGACCCTATAAAGCTTGACAATAATTTAA | 969 |
| 6884 | merguiensis | TTATACTATCAATTGTTAGTGTAACTTGGTTTTAATTAAA | |
| | | ATTTGTTGCGTTGGGGCGACGAGAATATAATAGGTAACTG | |
| | | TTCTTAAATATTTAATAACAAATATAATTGAAAATTAGTG | |
| | | TGATCCTCTATTAGCGATTAAAAGATTAAGTTACTTTAGG | |
| | | GATAACAGCGTAAT | |
| NC_02 | Metapenaeus | GGGGGACGATAAGACCCTATAAAGCTTTACAGTGTGCTAA | 970 |
| 6834 | ensis | TTAAATTATAAATTGTTAGTATAAACTTGGTTTGGTTAGT | |
| | | AGCTGTTTCGTTGGGGCGACGAAAATATAATTGATAGTAA | |
| | | CTGTTTTTCTTTTATAATAACAATAAATTTTTGGTAGAAA | |
| | | TTGATCCTTTATTAAAGATTAAAAGATTAAGTTACTTTAG | |
| | | GGATAACAGCGTAAT | |
| NC_01 | Acetes | GAGGGACGATAAGACCCTATAAAGCTTTACTGTTTAAATT | 971 |
| 7600 | chinensis | TTTTGATATTAGTATTAAATGATCCTCTAATAAAGATTAA | |
| | | AAATTTAAGTTACTTTAGGGATAACAGCGTAAT | |
| NC_01 | Macrobrachium | AGTGGGACGATAAGACCCTATAAAACTTTATATAAGTTTA | 972 |
| 5073 | nipponense | ATTTGGCTTACAATTTGAGTGAAAAGTAGCTTTATTAGGC | |
| | | TTATATTTCGTTGGGGAGATGTAGATGGGATAATCGACTG | |
| | | TAGAGGCCTGCATGCTATTTAATTTAATAATTATAATTAG | |
| | | TGTGTGATCCTTCTTTGTGGATTAAAAGTATAAGTTACTT | |
| | | TAGGGATAACAGCGTGAT | |
| NC_01 | Penaeus | AGGGGACGATAAGACCCTATAAAGCTTGACAATAAGTCGT | 973 |
| 2738 | californiensis | TTATATTATAAATTGTTAGTGTAACTTGATTTTAAACGGT | |
| | | GTTTGTTGCGTTGGGGCGACGGGAATATAAAAGGTAACTG | |
| | | TTCTTATAATATTTAATAACAGAAATATTTGGAAAATTAA | |
| | | TGATCCTTTCTTAAAGATCATAAGATTAAGTTACTTTAGG | |
| | | GATAACAGCGTAAT | |
| NC_01 | Macrobrachium | ATGGGACGATAAGACCCTATAAAACTTAATATAATCTAGT | 974 |
| 2217 | lanchesteri | TAATACTTGCGATTTAGGTAAAAAGGAGTTTTGTTTAGAT | |
| | | TATATTTCGTTGGGGAGATGAAGATATAATGTATAACTGT | |
| | | CTATTGAATAATATAATGCTAATTAGAGTTTGATCCTTCT | |
| | | TTGTGGATTAAAAGAATAAGTTACTTTAGGGATAACAGCG | |
| | | TAAT | |
| NC_03 | Pleoticus | AGGGGACGATAAGACCCTATAAAGCTTTACAATAAATTAA | 975 |
| 9964 | muelleri | TTAAATTATAAATTATAAGTTTAACTTAATTTAATTAGTA | |
| | | TTTGTTTCGTTGGGGCGACGAGAATATAATTATATAACTG | |
| | | TTCTTTTATTGTTATAACAAATTTAATTGGGTGAAAATTG | |
| | | ATCCTTTTATAAAGATTAAAAGATTAAGTTACTTTAGGGA | |
| | | TAACAGCGTAAT | |
| NC_03 | Metapenaeus | GGGGGACGATAAGACCCTATAAAGCTTTACAGTTTGTTGA | 976 |
| 9179 | affinis | TTAAATTATAAATTGTTAGTGTGAACTTAATTTATTTGAT | |
| | | AGCTGTTTCGTTGGGGCGACGAAAATATAATAAATAGTAA | |
| | | CTGTTTTTCTTTTATAATAACAATGATTTTTGGTAGTAAT | |
| | | TGATCCTTTATTAAAGATTAAAAGATTAAGTTACTTTAGG | |
| | | GATAACAGCGTAAT | |
| NC_03 | Hymenopenaeus | AAGGGACGATAAGACCCTATAAAGCTTTACAATAAGAAAA | 977 |
| 9169 | neptunus | TTAAACTATAAATTATTAGTATAACTTAATTTAATTGGTA | |
| | | TTTGTTTCGTTGGGGCGACGGGAATATAATAATATAACTG | |
| | | TTCTTTTATAAGTTTAACAAAATTAATTGGATAAAAATTG | |
| | | ATCCTTTAATAAAGATTAAAAGATTAAGTTACTTTAGGGA | |
| | | TAACAGCGTAAT | |
| NC_03 | Penaeus | AGGGGACGATAAGACCCTATAAAGCTTGACAATAATTTAA | 978 |
| 1366 | indicus | TTATACTATCAATTGTTAGTATAACTTGGTTTTAATTAAT | |
| | | ATTTGTTGCGTTGGGGCGACGAGAATATAATAGGTAACTG | |
| | | TTCTTAAATATTTAATAACAAATATAATTGGAAATTAGTA | |
| | | TGATCCTCTATTAGCGATTAAAAGATTAAGTTACTTTAGG | |
| | | GATAACAGCGTAAT | |
| NC_03 | Aristaeomorpha | AGGGGACGATAAGACCCTATAAAGCTTTACAATAAATTAA | 979 |
| 9153 | foliacea | ATATATTAAAATCTGTTAGTGTAACTTATTATATTTAGAG | |
| | | TTTGTTTCGTTGGGGCGACGAGAATATAATGAATAACTGT | |
| | | TCTTTTTAAAAATAGACAATAATAATTGGTTAAAGATTGA | |
| | | TCCTTTATTAAAGATTAAAAGATTAAGTTACTTTAGGGAT | |
| | | AACAGCGTAAT | |
| NC_03 | Solenocera | AGGGGACGATAAGACCCTATAAAGCTTTACAATAAGTTAA | 980 |
| 0280 | crassicornis | TTAAATTATAAATTGTTAGTATAACTTGGTTTAGTTATTA | |
| | | TTTGTTTCGTTGGGGCGACGAGAATATAATTATATATAAC | |
| | | TGTTCTTTTAAGAAGAAAACAAGGTTAATTGGATTAGAAT | |
| | | TGATCCTTTAATAAAGATTAAAAGATTAAGTTACTTTAGG | |
| | | GATAACAGCGTAAT | |
| NC_03 | Mierspen | AGGGGACGATAAGACCCTATAAAGCTTTACAATAAATTAA | 981 |
| 0277 | aeopsis | TTAAATTATAAATTGTTAGTTTAACTTAATTTAATTAGTG | |
| | hardwickii | TTTGTTTCGTTGGGGCGACGAGAATATAAATTATATAACT | |
| | | GTTCTTTTAGATAAATAACAAGGTTAATTGGAGGAAGATT | |
| | | GATCCTTTAATAAAGATTAAAAGATTAAGTTACTTTAGGG | |
| | | ATAACAGCGTAAT | |
| NC_04 | Penaeus | GGGGGACGATAAGACCCTATAAAGCTTGACAATAACTTCG | 982 |
| 0140 | latisulcatus | TTAAATTGTAAATTGTTAGTGTAACTTGATTTTAACTAGG | |
| | | GTTTGTTTCGTTGGGGCGACGGGAATATAATAAATAACTG | |
| | | TTCTTTTAAGTATAGTTACAAAAATGCTTGGTGAATAATT | |
| | | GATCCTCTACTAGAGATTAAAAGATAAAGTTACTTTAGGG | |
| | | ATAACAGCGTAAT | |
| MG82 | Penaeus | AGGGGACGATAAGACCCTATAAAGCTTGACAATAAGTTAA | 983 |
| 1354 | semisulcatus | TTATATTATAAATTGTTAGTATAACTTGATTTTAATTGAC | |
| | | GTTTGTTACGTTGGGGCGACGAGAATATAATGAGTAACTG | |
| | | TTCTTAAATGTTTATTGACAAATATAATTGGTATTTGATT | |
| | | GATCCTTTATTAAAGATTAAAAGATTAAGTTACTTTAGGG | |
| | | ATAACAGCGTAAT | |
| AF401 | Penaeussilasi | AGGGGACGATAAGACCCTATAAAGCTTGACAATAATTTAA | 984 |
| 305 | | TTATACTATCAATTGTTAGTATAACTTGGTTTTAATTAAG | |
| | | ATTTGTTGCGTTGGGGCGACGAGAATATAATAGGTAACTG | |
| | | TTCTTAAATATTTAATAACAAATATAATTGAAAATTAGTG | |
| | | TGATCCTCTATTAGCGATTAAAAGATTAAGTTACTTTAGG | |
| | | GATAACAGCGTAAT | |
| MG00 | Penaeus | AGGGGACGATAAGACCCTATAAAGCTTAACAATAAGCTGT | 985 |
| 1049 | isabelae | CTATATTATAAACTGTTAGTATAACTTGATACTAGATGGT | |
| | | ATTTGTTGCGTTGGGGCGACGAGAATATAAAAGGTAACTG | |
| | | TTCTTATAACATATAATAACAGAAATATCTGGAAATTTAA | |
| | | TGATCCTTTTTTAAAGATCATAAGATTAAGTTACTTTAGG | |
| | | GATAACAGCGTAAT | |
| NC_03 | Sicyonia | GGGGGACGATAAGACCCTATAAAGCTTTACAAACCCTTTT | 986 |
| 9168 | lancifer | TTTAGTTAAAAATTATAAGTGTAAACTAACTTAAAAATTA | |
| | | TTTGTTTCGTTGGGGCGACGAGAATATAATTTAGTAACTG | |
| | | TTCTTTTAAAATATTACAGTATTAGTTGATTGAAAATTGA | |
| | | TCCCTTAATAAGGATTAAAAGTTTAAGTTACTTTAGGGAT | |
| | | AACAGCGTAAT | |
| NC_02 | Metapenaeopsis | GAGGGACGATAAGACCCTATAAAGCTTTACAATTATTTAG | 987 |
| 9457 | dalei | TTAAGTTATAAATTGTTGGTCTAACTTAATTTAATTAATA | |
| | | TTTGTTTCGTTGGGGCGACGAGAATAAAATTAAGTAACTG | |
| | | TTCTTTTGTAAATAATTACAAATCTATTTGGTAAAAAATT | |
| | | GATCCTTTATTAAAGATTAAAAGATTAAGTTACTTTAGGG | |
| | | ATAACAGCGTAAT | |
| MK971 | Metapenaeopsis | AAGGGACGATAAGACCCTATAAAGCTTTACAATTAGTCAA | 988 |
| 537 | gerardoi | CTAAATCATAAATTATTGGTTTAACTTGGTTTAGTTGATG | |
| | | TTTGTTTCGTTGGGGCGACGAGAATATAATTAAAATAACT | |
| | | GTTCTTTAATAATTAATTACAGTTTTTTCTGATATAAAAT | |
| | | TGATCCTTTATTAAAGATTAAAAGATTAAGTTACTTTAGG | |
| | | GATAACAGCGTAAT | |
| KP059 | Parapenaeus | GGGGGACGATAAGACCCTATAAAGCTTTACAATTATTTAA | 989 |
| 279 | longirostris | TTAAATTATAAATTGTTAGTATAACTTAATTTAATTGATA | |
| | | TTTGTTTCGTTGGGGCGACGAGAATATAATTTTAAGTAAC | |
| | | TGTTCTTTTATGGTTAATTACAGATTTATTTGGGTAAAAA | |
| | | TTGATCCTTTATTAGAGATTAAAAGATTAAGTTACTTTAG | |
| | | GGATAACAGCGTAAT | |
| MK470 | Pandaluseous | GGGGGACGATAAGACCCTATAAAATTTTACAAAATTGGGA | 990 |
| 790 | | TTTTTTTTTGAATTAAGGTTTAAATTCTATAAGTTCTTAT | |
| | | TTGTTTTGTTGGGGCGGCAAAGATAAAAATTAGTAACTGT | |
| | | CATTTAATTAGAATAATTATAATTAGTTTAATTGATCCTT | |
| | | TATTAGAGATTAAAAGATTAAGTTACTTTAGGGATAACAG | |
| | | CGTAAT | |
| LC431 | Pandalus | GGGGGACGATAAGACCCTATAAAATTTTACAAAATCGGGA | 991 |
| 729 | miyakei | TTTTTTTTGAATTAAGGTTTAAGTTCTATAATTCCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATTAAGTTACTTTAGGGATAACAGC | |
| | | GTAAT | |
| MK470 | Pandalus | GGGGGACGATAAGACCCTATAAAATTTTACAAAATTGGAA | 992 |
| 785 | japonicas | TTTTTATTTGAATTAGGGTTTAAATTCTATGATTTCTTGT | |
| | | TTGTTGTGTTGGGGCGACAAAGATAAAAGTTAGTAACTGT | |
| | | CATTTAAGTAGAATAAGTATAATTAGTTTAGTTGATCCTT | |
| | | TATTAGAGATTAAAAGATTAAGTTACTTTAGGGATAACAG | |
| | | CGTAAT | |
| LC431 | Pandalus | GGGGGACGATAAGACCCTATAAAATTTTACAAAATCGGGA | 993 |
| 728 | glabrus | TCTTTTTTGAATTAAGGTTTAAATTCTATGATTCCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATTAAGTTACTTTAGGGATAACAGC | |
| | | GTAAT | |
| MK470 | Pandalus | GGGGGACGATAAGACCCTATAAAATTTTACAGAGTTGGGA | 994 |
| 796 | teraoi | CCTTTATTTTAAATTAGTGTTTAAATCTTATGGGTTCTTA | |
| | | TTTGTTTTGTTGGGGCGACAAAGATAAAATTTAGTAACTG | |
| | | TCATTTAATTAGAATAAGTATAATTAGTTTAGTTGATCCT | |
| | | TTATTAGAGATTAAAAGATTAAGTTACTTTAGGGATAACA | |
| | | GCGTAAT | |
| MK470 | Pandalus | GGGGGACGATAAGACCCTATAAAATTTTACAAAATTAGGA | 995 |
| 793 | ivanovi | TTTTTTTTTAAATTAAGGTTTAAATTCTATAAGTTCTTGT | |
| | | TTGTTGTGTTGGGGCGGCAAAGATAAAAATTAGTAACTGT | |
| | | CATTTAATCAGAATAAGTATAATTAGTTTAGTTGATCCTT | |
| | | TATTAGAGATTAAAAGATTAAGTTACTTTAGGGATAACAG | |
| | | CGTAAT | |
| MK470 | Pandalus | GGGGGACGATAAGACCCTATAAAATTTTACAAAATCGGGG | 996 |
| 784 | coccinatus | TTTTTTTTTAATTAAGGTTTAAATTCTGTAATTTCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATCAAGTTACTTTAGGGATAACAGC | |
| | | GTAAT | |
| MK470 | Pandalus | GGGGGACGATAAGACCCTATAAAATTTTACAGAGTCGGGT | 997 |
| 791 | formosanus | CTTTTTTTAAATTAGTGTTTAAATTCTGCAGGTTCTTATT | |
| | | TGTTTTGTTGGGGCGACAAAGATAAAATTTAGTAACTGTC | |
| | | ATTTAATCAGAATAGATATAATTAGTTTAGTTGATCCTTT | |
| | | ATTAGAGATTAAAAGATTAAATTACTTTAGGGATAACAGC | |
| | | GTAAT | |
| LC431 | Pandalus | GGGGGACGATAAGACCCTATAAAATTTTACAAAATTGGGA | 998 |
| 732 | princeps | TTTTTTTTGAATTAAGGTTTAAATTCTATAATTCCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATTAAGTTACTTTAGGGATAACAGC | |
| | | GTAAT | |
| MK470 | Pandalus | GGGGGACGATAAGACCCTATAAAATTTTACAGAGTCGGGA | 999 |
| 789 | chani | TCTTTTTTTAAATTAGTGTTTAAATTCTATGGGTTCTTAT | |
| | | TTGTTTTGTTGGGGCGACAAAGATAAAATTTAGTAACTGT | |
| | | CATTTAATTAGAATAAGTATAATTAGTTTAGTTGATCCTT | |
| | | TATTAGAGATTAAAAGATTAAGTTACTTTAGGGATAACAG | |
| | | CGTAAT | |
| LC431 | Pandalus | GGGGGACGATAAGACCCTATAAAATTTTACAAAATCGGAG | 1000 |
| 73 | houyuu | TTTTTTTTGAATTAAGGTTTAAATTCTTTAATTCCTTGTT | |
| | | TGTTGTGTTGGGGCGACAAAGATAAAAATTAGTAACTGTC | |
| | | ATTTAATTAGAATAAGTATAATTAGTTTAGTTGATCCTTT | |
| | | GTTAGAGATTAAAAGATTAAGTTACTTTAGGGATAACAGC | |
| | | GTAAT | |
| LC431 | Pandalus | GGGGGACGATAAGACCCTATAAAATTTTACAAAATTGGGA | 1001 |
| 730 | capillus | TTTTTTTTGAATTAAGGTTTAAATTCTATAATTCCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATTAAGTTACTTTAGGGATAACAGC | |
| | | GTAAT | |
| MK470 | Pandalus | GGGGGACGATAAGACCCTATAAAATTTTACAAAATTGGGA | 1002 |
| 786 | longirostris | TTTTTTTTGAATTAAGGTTTAAATTCTATAATTCCTTGTT | |
| | | TGTTGTGTTGGGGCGGCAAAGATAAAAATTAGTAACTGTC | |
| | | ATTTAATTAGAATAAGTATAATTAGTTTAGTTGATCCTTT | |
| | | ATTAGAGATTAAAAGATTAAGTTACTTTAGGGATAACAGC | |
| | | GTAAT | |
| MK470 | Pandalus | GGGGGACGATAAGACCCTATAAAATTTTACAAAATCGGAG | 1003 |
| 787 | ochotensis | TTTTTTTTGAATTAAGGTTTAAATTCTTTAATTCCTTGTT | |
| | | TGTTGTGTTGGGGCGACAAAGATAAAAATTAGTAACTGTC | |
| | | ATTTAATTAGAATAAGTATAATTAGTTTAGTTGATCCTTT | |
| | | GTTAGAGATTAAAAGATTAAGTTACTTTAGGGATAACAGC | |
| | | GTAAT | |
| AB244 | Pandalus | AGGGGACGATAAGACCCTATAAAATTTTACAAAATAGGGA | 1004 |
| 633 | latirostris | TTTTTTTTAAATTAAGGTTTAAATTCTATAAGTCTCTGTT | |
| | | TGTTGTGTTGGGGCGGCAAAGATAAAAATTAGTAACTGTC | |
| | | ATTTAATTAGAATAAGCATAATTAGTTTAGTTGATCCTTT | |
| | | ATTAGAGATTAAAAGATTAAGTTAGTTTAGGGATAACAGC | |
| | | GTAAT | |
| MK500 | Metapenaeus | GGGGGACGATAAGACCCTATAAAGCTTTACAGTGTATTAA | 1005 |
| 702 | brevicornis | TTAAATTTTAAATTGTTAGTATTAACTTAGTTTATTTAGT | |
| | | AACTGTTTCGTTGGGGCGACGAAAATATAATAAATAGTAA | |
| | | CTGTTTTTCTTTATAATATAACAAAAATTTTTGGAAGTAA | |
| | | TTGATCCTTTCTTAAAGATTAAAAGATTAAGTTACTTTAG | |
| | | GGATAACAGCGTAAT | |
| MK500 | Metapenaeus | GGGGGACGATAAGACCCTATAAAGCTTTACAGTGTATTAA | 1006 |
| 704 | dobsoni | TTAAATTTTAAATTGTTAGTATTAACTTAGTTTATTTAGT | |
| | | AACTGTTTCGTTGGGGCGACGAAAATATAATAAATAGTAA | |
| | | CTGTTTTTCTTTATAATATAACAAAAATTTTTGGAAGTAA | |
| | | TTGATCCTTTCTTAAAGATTAAAAGATTAAGTTACTTTAG | |
| | | GGATAACAGCGTAAT | |
| MK470 | Heterocarpus | GGGGGACGATAAGACCCTATAAAACTTTACAAGCGAAGAT | 1007 |
| 779 | hayashii | TTGTATTTTTTAAATTAAAGTATAACGTTATGTACTGTTC | |
| | | TGTTTGTTATGTTGGGGCGACAAAGATATAATAAGTAACT | |
| | | GTTAATTTTATTGAATAGTGATAATTAGTTTAATTGATCC | |
| | | TCTCTTAGAGATTACAAGATTAAGTTACTTTAGGGATAAC | |
| | | AGCGTAAT | |
| MK470 | Heterocarpus | AGGGGACGATAAGACCCTATAAAACTTTACAGATATAATT | 1008 |
| 780 | fascirostratus | ATACTTATTAAAATAAAGTTTAATAGTATTTATTATTTTA | |
| | | TCTGTTATGTTGGGGCGACAAAGATATAATAAAGTAACTG | |
| | | TTAGTTTTATTTGATAATAATAATTAGTTTAATTGATCCT | |
| | | TTATTAAAGATTATAAGAATAAGTTACTTTAGGGATAACA | |
| | | GCGTAAT | |
| NC_05 | Palaemon | AGGGGACGATAAGACCCTATAAAACTTTATAAATGAAGTA | 1009 |
| 0168 | adspersus | TGTAATCTCTAAATTATATTAAAATGTTGATAAGTATTTT | |
| | | ATTTATTTCGTTGGGGCGACGTTGATATAATTTGTAACTG | |
| | | TCTAAATGAATAATATAATCGTCATTATACTTTGATCCTT | |
| | | CTTTGTGGATTATAAGACTAAGTTACTTTAGGGATAACAG | |
| | | CGTAAT | |
| NC_05 | Palaemon | AGGGGACGATAAGACCCTATAAAACTTTATACATAGAATA | 1010 |
| 0266 | serratus | TAAAATCTTTAAATTATATTAAAATATTGATGGATATTTT | |
| | | ATGTATTTCGTTGGGGAGACGTTGATATAATTTGTAACTG | |
| | | TCTGAATGAATAATATAATTATCATTATAGTTTGATCCTT | |
| | | CTTTGTGGATTATAAGAATAAGTTACTTTAGGGATAACAG | |
| | | CGTAAT | |
| NC_04 | Palaemon | AGGGGACGATAAGACCCTATAAAACTTAATATTTATTTAT | 1011 |
| 5090 | sinensis | ATTTTGTTTTTAAATTTTAAATAAACTTTACAAAGTTTAG | |
| | | TATATATTTTGTTGGGGAGACATTGACATAATTAATAACT | |
| | | GTCTAATTTAAATGTTTATAGCTATTGTTATTATATGATC | |
| | | CTTCTTTGAGGATGAAAAGAGCAAGTTACTTTAGGGATAA | |
| | | CAGCGTAAT | |
| NC_03 | Palaemon | AGGGGACGATAAGACCCTATAAAACTTTATATAAATTAAG | 1012 |
| 9373 | capensis | AATTATTGTGAATTGGACTTAAATATTTTAATGCTTAATT | |
| | | TATATTTCGTTGGGGAGACGCTGATATAATTTGTAACTGT | |
| | | CAGTTAAATAAAATATTTATAATTAGTAAATTGATCTTTC | |
| | | AATGAGAATTTAAGAGAAGTTACTTTAGGGATAACAGCGT | |
| | | AAT | |
| NC_03 | Palaemon | GGGGGACGATAAGACCCTATAAAACTTGATAAAAAGTTTA | 1013 |
| 8117 | annandalei | TTTTAACTTTAAATTATATTTAAAAATTGTGAAGTTTTCT | |
| | | TTTTATTTTGTTGGGGCGACATTGATATAAATTGTAACTG | |
| | | TTTTAGCAAAAAGTATAATTATCATTATAGTTTGATCCTT | |
| | | CTTTGGGGATAAAAAGAATAAGTTACTTTAGGGATAACAG | |
| | | CGTGAT | |
| NC_02 | Palaemon | AAGGGACGATAAGACCCTATAAAACTTTATAAGAGATTTA | 1014 |
| 9240 | gravieri | TTTTATTTTTAAATTCTTCTTTAAAAATTGTGAAGTATTT | |
| | | CTCTTATTTTGTTGGGGCGACATTGATATAAGCTGTAACT | |
| | | GTTTAAATAAAAACTATAATTATCATTATTGTTTGATCCT | |
| | | TCTTTGTGGATAAAAAGACTAAGTTACTTTAGGGATAACA | |
| | | GCGTAAT | |
| NC_02 | Palaemon | GTGGGACGATAAGACCCTATAAAACTTTATATGTTGCCTG | 1015 |
| 7601 | serenus | CTTTTTGTTAAAATTCTACCTAAAATTTGAAAAGCACTCT | |
| | | ACATATTTTGTTGGGGAGACATTGATATAAATTGTAACTG | |
| | | TCTAAATAAAAAATATAATTATCATTATAATCTGATCCTT | |
| | | CTTTGTGGATAAAAAGAATAAGTTACTTTAGGGATAACAG | |
| | | CGTGAT | |
| NC_01 | Palaemon | AGGGGACGATAAGACCCTATAAAACTTAATAAAAGGTTAA | 1016 |
| 2566 | carinicauda | TTTTAACTATGAATTGTATTTAAAAGTTGTGAAATTCTCT | |
| | | TTTTATTTTGTTGGGGCGACATTGATATAAGCTGTAACTG | |
| | | TCTTGATAATAAAATAGTTATCACTATAAGCTGATCCTTT | |
| | | TTTAGGGATAATAAGTATAAGTTACTTTAGGGATAACAGC | |
| | | GTAAT | |
| KT959 | Palaemon | AAGGGACGATAAGACCCTATAAAACTTGACACGCCACTGT | 1017 |
| 474 | pugio | ATTTAGCTTTAAATTTTAATCTAAAATTGTAAGGGTAGCT | |
| | | GTGTGTTTTGTTGGGGAGACATTGATATAAGTAGTAACTG | |
| | | TCTGATCAAAAATTATAATTATAATTAGCATATGATCCCT | |
| | | ATTTTAGGATTATAAGAATAAGTTACTTTAGGGATAACAG | |
| | | CGTAAT | |
| KF923 | Palaemon | AGGGGACGATAAGACCCTATAAAACTTCACATGAAACGTT | 1018 |
| 713 | pandaliformis | GTAGTTTATGGATCTTGTTTAATGTAATTTCAGCGTTTTT | |
| | | TGTTTTGTTGGGGCGACATGGATAAAAAGTAACTGTCTAT | |
| | | TAAAATTCAATAACAATAATTAGTTTGTGATCCTTTATTA | |
| | | AGGATTAAAAGAATAAGTTACTTTAGGGATAACAGCGTGA | |
| | | T | |
| MT340 | Palaemon | AGGGGACGATAAGACCCTATAAAACTTTATGCACGACATG | 1019 |
| 087 | elegans | TCTTGTCTTTAAATTATATCTAAATATTGGTTGGTTAAAA | |
| | | GTGCATTCCGTTGGGGAGACGTTGATATAAGCTGTAACTG | |
| | | TTTTATTAAATAGAATAATTATTATTATAATTTGATCCTT | |
| | | CTTTGTGGATAAAAAGAATAAGTTACTTTAGGGATAACAG | |
| | | CGTAAT | |
| MT340 | Palaemon | ATTACGCTGTTATCCCTAAAGTAACTTATTCTTATAACCC | 1020 |
| 091 | longirostris | ACAAAGAAGGATCAAAATATAATGATAGTTATTTTATTCA | |
| | | CTCAGACAGTTACAAATTATATCAACGTCTCCCCAACGAA | |
| | | ATATATTAAGAATTTATCAGTATTTTAATATAATTTAAAG | |
| | | ATTATATACTTTATATATAAAGTTTTATAGGGTCTTATCG | |
| | | TCCCTC | |
| JQ042 | Palaemon | GTGGGACGATAAGACCCTGTAAAACTTTATACGCATAGTA | 1021 |
| 296 | peringuey | TTTAGCCTTTAAATTATATTAAAATATTGATATATACTTT | |
| | | GGGTATTTCGTTGGGGAGACGTTGATATAATTTGTAACTG | |
| | | TCTAATTAAATAAAATAATTATCATTATACTTTGATCCTT | |
| | | CTTTGTGGGTTATAAGAATAAGTTACTTCAGGGATAACAG | |
| | | CGTAAT | |
| KP725 | Palaemon | GGGGGACGATAAGACCCTATAAAACTTTATGTGATTTGTG | 1022 |
| 611 | debilis | GTTATTTATTAAAATTTTACTTAAAATTTAGAAAATCTCA | |
| | | TTTACATTTTGTTGGGGAGACATAGATATAATTTGTAACT | |
| | | GTCTGTTGTAAAAATATAGCTATCGTTAGAATTTGATCCC | |
| | | TCTGTGGGGATAATAAGAATAAGTTACTTTAGGGATAACA | |
| | | GCGTAAT | |
| KF923 | Palaemon | AGGGGACGATAAGACCCTATAAAACTTTATATAAAATAAG | 1023 |
| 720 | carteri | TATATCTACATATTATGTTAATAGGGGTATGCTATTTTAT | |
| | | ATTTTGTTGGGGCGACATAGATATGTAAGCAAATTAACTG | |
| | | TTTATTAAAATAAAATAATAATAATTAGTTTATGATCCTT | |
| | | TAGTGAAGATTAAAAGAGTAAGTTACTTTAGGGATAACAG | |
| | | CGTGAT | |
| JN674 | Palaemon | AGGGGACGATAAGACCCTATAAAACTTGATATAGAGCTGA | 1024 |
| 344 | ritteri | CTTTAGTTTTAAATTTTAAAATAAAATTGTAAAGTAAACT | |
| | | TTATATTTTGTTGGGGAGACATTGATATAAGTAGTAACTG | |
| | | TCTGTTAAAAATTATAATTATATTTAGTTTTTGATCCTTC | |
| | | TTTGTGGATTAGAAGAATAAGTTACTTTAGGGATAACAGC | |
| | | GTGAT | |
| KC515 | Palaemon | AGGGGACGATAAGACCCTATAAAACTTAATAAAAGGTTAA | 1025 |
| 036 | orientis | TTTTAACTATGAATTGTATTTAAAAGTTGTGAAATTCTCT | |
| | | TTTTATTTTGTTGGGGCGACATTGATATAAGCTGTAACTG | |
| | | TCTTGATAATAAAATAGTTATCACTATAAGCTGATCCTTT | |
| | | TTTAGGGATAATAAGTATAAGTTACTTTAGGGATAACAGC | |
| | | GTAAT | |
| DQ194 | Macrobrachium | TGTGTAATTTATTACAAAGTCTAGCCTGCCCACTGATTTA | 1026 |
| 924 | gracilirostre | AGGGGACGATAAGACCCTATAAAACTTAATATAAGTTGGG | |
| | | CTTAATTTATGATTTAGGTGAAAAATGATTTTGTTTGGTT | |
| | | TATATTTCGTTGGGGAGATGAAGATATAAAATGTAACTGT | |
| | | CTATTTAAATTTAATAATATTAATTAGTATGTGATCCTTC | |
| | | TTTGTGGATTATAAGAATAAGTTACTTTAGGGATAACAGC | |
| | | GTAAT | |
| KT959 | Palaemon | AAGGGACGATAAGACCCTATAAAACTTAACACATTGTTAT | 1027 |
| 473 | vulgaris | TTCTAGCTTTGAATTTTAACCTAAAATTTTAGAGATAATC | |
| | | TTGTGTTTTGTTGGGGAGACATTGATAAAACTAGTAACTG | |
| | | TCTAAGTAAAAATTATAATTATAATTAGCATGTGATCCCT | |
| | | CTATGAGGATTAGAAGAATAAGTTACTTTAGGGATAACAG | |
| | | CGTAAT | |
| JQ042 | Palaemon | GGGGGACGATAAGACCCTATAAAACTTTATAAAAGACTAA | 1028 |
| 295 | serrifer | TTTCATTTTTAAATTCTGTTAAAAAATTATGAAATTTTTC | |
| | | TTTTATTTTGTTGGGGCGACATTAATATAAGCTGTAACTG | |
| | | TTTAAATAAAAATTATAATTATCATTATTGTTTGATCCCT | |
| | | CTTTGAGGATAAGAAGACTAAGTTACTTTAGGGATAACAG | |
| | | CGTAAT | |
| MT340 | Palaemon | GGGGGACGATAAGACCCTATAAAACTTTATACACGACTCA | 1029 |
| 090 | varians | GTTCATCTTTAAATTTTAATTAAATTAAAGTGAAATGGGA | |
| | | CGTGTATTTCGTTGGGGAGACGTTGATATAAACTGTAACT | |
| | | GTCTAATTGAAAAATATAAAAATAATTATTATATGATCCT | |
| | | TCTTTGTGGATAAAAAGATTAAGTTACTTTAGGGATAACA | |
| | | GCGTAAT | |
| JQ042 | Palaemon | AAGGGACGATAAGACCCTATAAAACTTTATAAGAGATTTA | 1030 |
| 297 | macrodactylus | TTTTATTTTTAAATTCTTCTTTAAAAATTGTGAAGTAATT | |
| | | CTTTTATTTTGTTGGGGCGACATTGATATAAGCTGTAACT | |
| | | GTTTAAATAAAAACTATAATTATCATTATTGTTTGATCCT | |
| | | TCTTTGTGGATAAAAAGACTAAGTTACTTTAGGGATAACA | |
| | | GCGTAAT | |
| KC515 | Palaemon | GGGGGACGATAAGACCCTATAAAACTTTATATAAATATAG | 1031 |
| 035 | tonkinensis | GTTTTTTTTAATTTTACTAAAATATTAGAGATCCTTATTT | |
| | | ATATTTTGTTGGGGAGACATTGATATAAGCAGTAACTGTC | |
| | | TGATTAAAAATAATAATGATATTTATAAAGTGAACCCTCA | |
| | | CTGAGGATTAAAAGAATAAGTTACTTTAGGGATAACAGCG | |
| | | TAAT | |
| JQ042 | Palaemon | CGGGGACGATAAGACCCTATAAAACTTTATACATAAAGTG | 1032 |
| 294 | xiphias | TGTAATCTCTATATTATATTGAAATGTCGATAAGTACTCT | |
| | | ATTTATTTCGTTGGGGAGAGGTCCATATACCTTGTAACTG | |
| | | TCTGAGTGAACAAAATAATTATCATTATATTTTGATCCTT | |
| | | CTTTGTGGATTATAAGAGTAAGTTACTTTAGGGATAACAG | |
| | | CGTAAT | |
| KF923 | Palaemon | TGGGGACGATAAGACCCTATAAAACTTTATATAAAATATA | 1033 |
| 729 | ivonicus | TTTGTTTATAAATTATGTTTATAAGAAATTTATTATTTTA | |
| | | TATTTTGTTGGGGCGACATAAATAAAACATGTAACTGTTT | |
| | | ATATAAATAAAATAATGATAATTAAATTTTGATCCTTTAA | |
| | | TAAGGATTACAAGAATAAGTTACTTTAGGGATAACAGCGT | |
| | | AAT | |
| JN674 | Palaemon | GGGGGACGATAAGACCCTGTAAAACTTTATACGTAGGGTA | 1034 |
| 340 | pacificus | TTTAGTCTTTAAATTATACTAAATTGTTGATATATATCTT | |
| | | AGGTATTTCGTTGGGGAGACGTGGATATAATTTGTAACTG | |
| | | TCTAATTAATAATATAATTATCTTTATATTTTGAACCTTC | |
| | | TTTGTGGGTTATAAGAATAAGTTACTTCAGGGATAACAGC | |
| | | GTAAT | |
| JN674 | Palaemon | GTGGGACGATAAGACCCTATAAAACTTTATATGAATTGAG | 1035 |
| 352 | atrinubes | ATTATTTGTCAAAATTTTAATTAAAATTTAAAAGATCTTA | |
| | | TTTATATTTTGTTGGGGAGACATTGATATAACTTGTAACT | |
| | | GTCTAGTTGAAAAATATAGTTATTGCTAGAATTTGATCCC | |
| | | TCTATGAGGATAAAAAGAATAAGTTACTTTAGGGATAACA | |
| | | GCGTGAT | |
| KF923 | Palaemon | GTGGGACGATAAGACCCTATAAAACTTTACATGTGGTACG | 1036 |
| 725 | intermedius | CTTTTTTTTAAAATTTTAATTAAACTTTGGAAAGTGTTCT | |
| | | TCATGTTTTGTTGGGGAGACATTGATATAATTTGTAACTG | |
| | | TCTAAATAAAACATATAATAATCATTAGAATTTGATCCTT | |
| | | CTTTGTGGATAAAAAGAATAAGTTACTTTAGGGATAACAG | |
| | | CGTAAT | |
| KC515 | Palaemon | GGGGGACGATAAGACCCTATAAAACTTTACATAAGTTACA | 1037 |
| 043 | concinnus | TTTAGTTTGTGAATAAAGTAAAAAACTGTTTTAGATACCT | |
| | | TATGTTTCGTTGGGGAGACGTTTATATAACTGTAACTGTC | |
| | | AGATAAATTTAATAATTCTAATTAGTATATGATCCTTCTG | |
| | | TGGGGATTATAAGAGTAAGTTACTTTAGGGATAACAGCGT | |
| | | GAT | |
| KF923 | Palaemon | GGGGGACGATAAGACCCTATAAAACTTTATATAAAATATG | 1038 |
| 716 | yuna | TTTGTTTATAAATTGCGTTTAAAGGAAATTTATTGTTTTA | |
| | | TATTTTGTTGGGGCGACATAAATAAAATTTGTAACTGTTT | |
| | | ATATAAATAAAATAACGATAATTAGATTTTGATCTTTTAA | |
| | | TAAGGATTATAAGAATAAGTTACTTTAGGGATAACAGCGT | |
| | | AAT | |
| JQ042 | Palaemon | GGGGGACGATAAGACCCTATAAAACTTTATACGCTGTTTA | 1039 |
| 306 | antennarius | ATTTGTCTTTGAATTTTAAATAAATTAAAGTGAATTAGCC | |
| | | AGTGTATTTCGTTGGGGAGACGTTAATATAAATTGTAACT | |
| | | GTTTAATTAAAAAATATAAATATAATTATGGTATGATCCT | |
| | | TCTTTGTGGATTAAAAGATTAAGTTACTTTAGGGATAACA | |
| | | GCGTAAT | |
| JN674 | Palaemon | GTGGGACGATAAGACCCTATAAAACTTTATATGTGGCCTG | 1040 |
| 333 | dolospinus | CTTTTTCTTAAAATTTCAGATAAACTTTGAAAAGCAGTTC | |
| | | TCGTATTTTGTTGGGGAGACATTGATATAAATCGTAACTG | |
| | | TCTGAATAAAATATATAGTAATCACTATAATTTGATCCTC | |
| | | CTATGCGGCTAAAAAGAACAAGTTACTTTAGGGATAACAG | |
| | | CGTAAT | |
| KF923 | Palaemon | GGGGGACGATAAGACCCTATAAAACTTTACATAAAAAACT | 1041 |
| 714 | gracilis | ATAGTCTATGAATTATATTTAAGGGGAGTCTAGTCTTTTG | |
| | | TGTTTTGTTGGGGCGACATGGATAAAAAGTAACTGTCTAT | |
| | | TAAAATTTAATAATAATAATTAGTTTTTGATCCTTTAGTA | |
| | | TGGATTAAAAGAATAAGTTACTTTAGGGATAACAGCGTGA | |
| | | T | |
| JN674 | Palaemon | AAGGGACGATAAGACCCTATAAAACTTGATACGCCGTTAT | 1042 |
| 354 | mundusn | GCTTAGCTTTAAATTTTAATCTAAAATTGTAAGGGTGGCT | |
| | ovus | GTGTGTTCTGTTGGGGAGACATTGATATAAATAGTAACTG | |
| | | TCTGATTAAAAAATATAATTATAATTAGTATGTGATCCTT | |
| | | CTTTGTGGATTATAAGAGTAAGTTACTTTAGGGATAACAG | |
| | | CGTAAT | |
| KF923 | Palaemon | ATGGGACGATAAGACCCTATAAAACTTTATATGTTATATA | 1043 |
| 712 | suttkusi | TTTTATTTTTGAATTATTTTAAAATATTGTTTAATGTTTT | |
| | | ACATATTTCGTTGGGGAGACGTTGACATAAAATGTAACTG | |
| | | TTTAAATAAAAAATATAATTATCATTATATTTTGATCCTT | |
| | | CTTTGTGGATAAAAAGAATAAGTTACTTTAGGGATAACAG | |
| | | CGTAAT | |
| JQ042 | Palaemon | GGGGGACGATAAGACCCTATAAAACTTTATACATTATTTA | 1044 |
| 299 | zariquieyi | GTTCGTCTTTAAATTTTAAGTAAATTATAGTGAATTGATA | |
| | | TGTGTATTTCGTTGGGGAGACGTTGATATAAATTGTAACT | |
| | | GTCTAATTAAAAAATATAAATATGATTATGATGCGATCCT | |
| | | TCTTTGTGGATAAAAAGATTAAGTTACTTTAGGGATAACA | |
| | | GCGTAAT | |
| JN674 | Macrobrachium | GTGGGACGATAAGACCCTATAAAACTTTATATGTCGTTTG | 1045 |
| 353 | australiense | CTTTTTCTTAAAATTTTATGTAAACTTTGAGAAGCATTTC | |
| | | TCATATTTTGTTGGGGAGACATTGATATAAGCTGTAACTG | |
| | | TCTAAATAAAATATATAATAATCATTAGAATTTGATCCTT | |
| | | CTTTGTGGATAAAAAGAATAAGTTACTTTAGGGATAACAG | |
| | | CGTAAT | |
| JN674 | Palaemon | GGGGGACGATAAGACCCTATAAAACTTTATATTTTAATTT | 1046 |
| 345 | semmelin | TTTGCTTTAGATTATAGATTAAAATTGCAAAGTTATTTAA | |
| | kii | ATATTTCGTTGGGGCGACGTTGATAAAATCTTAACTGTCT | |
| | | GTGTAAAAGTTATAGTAATTACTAGAATATGATCCTTCTT | |
| | | TTGAGGATAACAAGAATAAGTTACTTTAGGGATAACAGCG | |
| | | TAAT | |
| JN674 | Palaemon | GTGGGACGATAAGACCCTATAAAACTTTATATGTTGCCTG | 1047 |
| 337 | litoreus | CTTTTTGTTAAAATTCTATCTAAAATTTGAAAAGCACTCT | |
| | | ACATATTTTGTTGGGGAGACATTGATATAAATTGTAACTG | |
| | | TCTAAATAAAAAATATAATTATCATTATAATTTGATCCTT | |
| | | CTTTGTGGATAAAAAGAATAAGTTACTTTAGGGATAACAG | |
| | | CGTGAT | |
| LC425 | Palaemon | AAGGGACGATAAGACCCTATAAAACTTAATATAAATTATA | 1048 |
| 608 | septemtri | CTATATTTTTAAATTATAAATAATCATGATAAAGTTTAGT | |
| | onalis | TTATATTTTGTTGGGGAGATATTGATATAAATTGTAACTG | |
| | | TCTAATTTAAAAAAAATAATAATCATTATAATGTGATCCT | |
| | | TCATTGAGGATAAAAAGAATAAGTTACTTTAGGGATAACA | |
| | | GCGTAAT | |
| JN674 | Palaemon | AAGGGACGATAAGACCCTATAAAACTTTATAAGAGATTTG | 1049 |
| 335 | guangdon | TTTTATTTTTAAATTCTTCTTTAAAAGTTGTGAAGTAATT | |
| | gensis | CTTTTATTTTGTTGGGGCGACATTGATATAAGCTGTAACT | |
| | | GTTTAAATAAAAACTATAATTATCATTATTGTTTGATCCT | |
| | | TCTTTGTGGATAAAAAGACTAAGTTACTTTAGGGATAACA | |
| | | GCGTAAT | |
| KF923 | Palaemon | GGGGGACGATAAGACCCTATAAAACTTTACATTAAAAACT | 1050 |
| 715 | hancocki | ATAGTCTATAAATTATATTTAAGGGGAGTCTAGCGTTTAT | |
| | | ATGTTTTGTTGGGGCGACATGGATAAAAAGTAACTGTCTA | |
| | | TAAAAATTTAATAATAATAATTAGTTTTTGATCCTTTATT | |
| | | ATGGATTAAAAGAATAAGTTACTTTAGGGATAACAGCGTG | |
| | | AT | |
| KC515 | Palaemon | GGGGGACGATAAGACCCTATAAAACTTAATAAAAGGTTTA | 1051 |
| 037 | vietnamicus | TTTTAACTATAAATTATATTTAAAAGTTGTGAAATCTTCT | |
| | | TTTTATTTTGTTGGGGCGACATTGATATAAACTGTAACTG | |
| | | TCTTAATAATAAAATAATTATCATTATGAGCTGATCCTTT | |
| | | TTTAGGGATAATAAGTATAAGTTACTTTAGGGATAACAGC | |
| | | GTAAT | |
| JQ042 | Palaemon | AAGGGACGATAAGACCCTATAAAACTTGATACGCCGTTAT | 1052 |
| 303 | texanus | GCTTAGCTTTAAATTTTAATCTAAAATTGTAAGGATGGCT | |
| | | GTGTGTTCTGTTGGGGAGACATTGATATAAATAGTAACTG | |
| | | TCTGATTAAAAAATATAATTATAATTAGTATGTGATCCTT | |
| | | CTTTGTGGATTATAAGAGTAAGTTACTTTAGGGATAACAG | |
| | | CGTAAT | |
| JN674 | Palaemon | AAGGGACGATAAGACCCTATAAAACTTTATAAGAGACTTA | 1053 |
| 339 | ortmanni | TTTTATTTTTAAATTCTTTTTAAAAATTATAAAATATTTC | |
| | | TTTTATTTTGTTGGGGCGACATTGATATAACCTGTAACTG | |
| | | TTTAAATAAAAATTATAATTATCATTATTGTTTGATCCTT | |
| | | CTTTGTGGATAAAAAGAATAAGTTACTTTAGGGATAACAG | |
| | | CGTAAT | |
| JQ042 | Palaemon | GGGGGACGATAAGACCCTATAAAACTTTATACGTTACCCT | 1054 |
| 302 | turcorum | ATTCATCTTTGAAGTTTAGATAAATTATAGTGAATGGGGG | |
| | | GGTGTATTTCGTTGGGGAGACGTTGATATAATCTGTAACT | |
| | | GTCTAATTGAAAAATATAAATATAATTAGTACATGATCCT | |
| | | TCTTTGTGGATAAAAAGATTAAGTTACTTTAGGGATAACA | |
| | | GCGTAAT | |
| KF923 | Palaemon | AAGGGACGATAAGACCCTATAAAACTTTATATGTAAGGTA | 1055 |
| 718 | kadiakensis | TTTTGTTTTTAAATTATTTTGAAATATTAGTTAATATTTT | |
| | | ACATATTTCGTTGGGGAGACGTTGATATAACATGTAACTG | |
| | | TCTAAATAAAAAAAATAATTATTATTATATTTTGATCCTT | |
| | | CTTTGTGGATAAAAAGAATAAGTTACTTTAGGGATAACAG | |
| | | CGTAAT | |
| EU493 | Macrobrachium | GAGGGACGATAAGACCCTATAAAACTTTATATAGACTTAA | 1056 |
| 137 | asperulum | TTTAACTTGTGACTTAAGTGAAAAATAGTTTTGTTGAGTT | |
| | | TATATTTTGTTGGGGAGATGTAGATATAATATGTAACTGT | |
| | | CTATTTAATTTAATAATTATAATTAGTGTTTGATCCTTCT | |
| | | TTGTGGATTAAAAGTGTAAGTTACTTTAGGGATAACAGCG | |
| | | TGAT | |
| GU987 | Macrobrachium | ATGGGACGATAAGACCCTATAAAACTTAATATAAATTTAG | 1057 |
| 057 | australe | TTTAATTTGTGACATAAGTGAAAAGTGGTTATACTTCATT | |
| | | TATATTTCGTTGGGGAGATGTAGATATAAAAGTAACTGTC | |
| | | TATTAAATTTAATAGCCATAGTTAGTAATTGATCCTTCTT | |
| | | TGGGGATTAAAAGAGTAAGTTACTTTAGGGATAACAGCGT | |
| | | GAT | |
| MH253 | Macrobrachium | GTGGGACGATAAGACCCTATAAAACTTGATATAAGTTGAA | 1058 |
| 292 | olfersii | CTTGATTTGTGATTTAAGTGAAAAGCAGTTTTGTTTGACT | |
| | | TATATTTTGCTGGGGCGGCATAGATATAAAATGTAACTGT | |
| | | CTATTAAATTTAATAGTGCTAATTAGTATTTGATCCTTCC | |
| | | TTGTGGATTAAAAGAATAAGTTACTTTAGGGATAACAGCG | |
| | | TGAT | |
| GU929 | Macrobrachium | GAGGGACGATAAGACCCTATAAAACTTAACATAAGTTTTG | 1059 |
| 448 | jelskii | TTTTATTTATAATTTTAGTGTAAAATGGTTTTACTAAGTT | |
| | | TATGTTTCGTTGGGGAGATGAAGATATAAAGAGTAACTGT | |
| | | CTTTTGAATAGAATAATTATAATTAGTTTTTGATCCTTCT | |
| | | TTGGGGATTAAGAGAATAAGTTACTTTAGGGATAACAGCG | |
| | | TGAT | |
| KJ544 | Macrobrachium | GGGGGACGATAAGACCCTATAAAACTTTATATAATCTAAA | 1060 |
| 746 | villosimanus | CTTGACTTGGGATTTAAGTGAAAAGTAGTTTTGTTCGGTT | |
| | | TATATTTCGTTGGGGAGATGAAGATATAATTTGTAACTGT | |
| | | CTATTGAATATTATAGTATTAATTAGTATTTGATCCTTCT | |
| | | TTGAGGATTAGGAGAATAAGTTACTTTAGGGATAACAGCG | |
| | | TGAT | |
| KP763 | Macrobrachium | GTGGGACGATAAGACCCTATAAAACTTAATATAAGTTAAA | 1061 |
| 693 | equidens | TTGGATTTGTGATTTCAGTAAAAAGTGGTCCTTTTAAGTT | |
| | | TATATTTCGTTGGGGAGATGTAGATATAATGTGTAACTGT | |
| | | TTATAAAACTAAATAATCATAATTAGTTTGTGATCCTGCA | |
| | | GATGTGGATTGAAAGAGTAAGTTACTTTAGGGATAACAGC | |
| | | GTAAT | |
| JX466 | Macrobrachium | GAGGGACGATAAGACCCTATAAAACTTAATATAAATTGTA | 1062 |
| 930 | potiuna | ATTTTGTTTATGATTTAGGTGGAAAATAATTTAATTTAAT | |
| | | TTATATTTCGTTGGGGAGATGTAGATATAAAATGTAACTG | |
| | | TCTATAAAATTTAATAATTATTGTTAGTGGTTGATCCTTC | |
| | | TTTGGGGATTAAAAGAATAAGTTACTTTAGGGATAACAGC | |
| | | GTAAT | |
| MK782 | Macrobrachium | GAGGGACGATAAGACCCTATAAAACTTGATATAATTTGAA | 1063 |
| 955 | malcolmsonii | CTTGACTTGTGATTTGAGTGAAAAATAGTCTTGTTTGGTT | |
| | | TATATTTCGTTGGGGAGATGAAGATATAATGTGTAACTGT | |
| | | CTATTGAATTTTATAGTATTAATTAGAATTTGATCCTTCT | |
| | | TTGAGGATTAGGAGAATAAGTTACTTTAGGGATAACAGCG | |
| | | TGAT | |
| KC515 | Macrobrachium | AGGGGACGATAAGACCCTATAAAACTTTATATAAAGTTCA | 1064 |
| 041 | superbum | TATTCGATTAAATTTTAATGTTAATAAAAATTGGATGTTA | |
| | | GCTTTATATTTTGTTGGGGCGACAAAAATAAAATTGATAG | |
| | | CAACTGTTTAAATAATTAGACAAAAATATTTGATTATATA | |
| | | ATTAAATGATCCTAATTAAAGATTAAAAGTTTAAGTTACT | |
| | | TTAGGGATAACAGCGTTAT | |
| KM610 | Macrobrachium | GTGGGACGATAAGACCCTATAAAACTTGATATAAGTTTGA | 1065 |
| 135 | striatum | TTGGATTTGTGATTTCAGTAAAAAATGGTCCTCTGAGGCT | |
| | | TATATTTCGTTGGGGAGATGTAGATATAATATGTAACTGT | |
| | | TTGTAAAATTAAATAATTATAATTAGTTTGTGATCCTGCC | |
| | | GTGTGGATTGAAAGAGTAAGTTACTTTAGGGATAACAGCG | |
| | | TAAT | |
| JF310 | Macrobrachium | AAGGGACGATAAGACCCTATAAAACTTGATACGAGTTTAG | 1066 |
| 722 | latidactylus | CTTAATTTGTGATTTATGTGAAAAGTAGTTTTGCTTGGTT | |
| | | CGTATTTCATTGGGGAGATGTAGATATAATCTGTAACTGT | |
| | | CTATGAAATTTAATAATCATAATTAGTAGTTGATCCTTCT | |
| | | TTGTGGATTAAAAGAGTAAGTTACTTTAGGGATAACAGCG | |
| | | TGAT | |
| JQ805 | Macrobrachium | GTGGGACGATAAGACCCTATAAAACTTGATATAAGTGAAC | 1067 |
| 822 | hancocki | CTTGATTTGTGATTTGTGTGAAAAATAGTGTTGCTTTACT | |
| | | TATATTTTGCTGGGGCGGCATAGATATAAAATGTAACTGT | |
| | | CTGTTAAATTTAATAGTGTTAATTAGTTTTTGATCCTTCC | |
| | | TTGTGGATTAGAAGAATAAGTTACTTTAGGGATAACAGCG | |
| | | TGAT | |
| GU929 | Macrobrachium | GGGGGACGATAAGACCCTATAAAACTTGATATTGATCTGG | 1068 |
| 449 | acanthurus | CATGATTTGCGATTTTAGTGAAAAGTAGTTTTGTTTGATC | |
| | | TATATTTCGTTGGGGAGATGAAGATATAAATTGTAACTGT | |
| | | CTATTTAATTTAATAATTATAGTTAGTGTTTGATCCTTCT | |
| | | TTGTGGATTAAGAGATTAAGTTACTTTAGGGATAACAGCG | |
| | | TGAT | |
| DQ194 | Macrobrachium | GTGGGACGATAAGACCCTATAAAACTTTATATAAGTTTAA | 1069 |
| 931 | inflatum | TTTGGCTTACAATTTGAGTGAAAAGTAGCTTTATTAGGTT | |
| | | TATATTTCGTTGGGGAGATGTAGATATAATGTGTAACTGT | |
| | | CTGTTTAATTTAATAATTATAATTAGTGTGTGATCCTTCT | |
| | | TTGTGGATTAAAAGTATAAGTTACTTTAGGGATAACAGCG | |
| | | TGAT | |
| JQ805 | Macrobrachium | GTGGGACGATAAGACCCTATAAAACTTGATATAAGTGAAG | 1070 |
| 801 | crenulatum | CTTGATTTGTGATTTGGGTGAAAAATAGTGTTGCTTTGCT | |
| | | TATATTTTGCTGGGGGGGCATGGATATAAAATGTAACTGT | |
| | | CTGTTAAATTTAATAGTGTTAATTAGTTTATGATCCTTCT | |
| | | TTGTGGATTAAAAGAATAAGTTACTTTAGGGATAACAGCG | |
| | | TGAT | |
| KM101 | | GTGGGACGATAAGACCCTATAAAACTTTATACAGGTTGGG | 1071 |
| 474 | carcinus | TCAAACTTGTGATTTGTGTGAAAAGTAGTTTTATTTGATC | |
| | | TGTATTTTGTTGGGGCGACATAGATATAATGTGTAACTGT | |
| | | CTATTAAATTAAATAGTGTTAATTAGTCTTTGATCCTTCT | |
| | | TTGTGGATTAGAAGAATAAGTTACTTTAGGGATAACAGCG | |
| | | TGAT | |
| GU929 | Macrobrachium | GTGGGACGATAAGACCCTATAAAACTTTATACAGGTTGGG | 1072 |
| 463 | americanum | TTCAATTTGTGATTTGTGTGAAAAGTAGTTTTATTCGGTC | |
| | | TGTATTTTGTTGGGGCGACATAGATATAATATGTAACTGT | |
| | | CTATCAAATTAAATAGTGTTAATTAGTTTTTGATCCTTCT | |
| | | TTGTGGATTAGAAGAATAAGTTACTTTAGGGATAACAGCG | |
| | | TGAT | |
| EU493 | Macrobrachium | GTGGGACGATAAGACCCTATAAAACTTGATATATGCTGAG | 1073 |
| 143 | latimanus | TTTAATTTATGATATAAGTGAAAAGTAGTTATACTTGGTT | |
| | | TATATTTCGTTGGGGAGATGTAGATATAAATTGTAACTGT | |
| | | CTATTTAATTTAATAATGTTAATTAGTGTGTGATCCCTCT | |
| | | TTGGGGATTAAAAGAATAAGTTACTTTAGGGATAACAGCG | |
| | | TGAT | |
| FM986 | Macrobrachium | GTGGGACGATAAGACCCTATAAAACTTGATATAAGCTTGA | 1074 |
| 629 | mammillo | TTTAATTTGCGATTATTAGTGAAAAGTAGTTTTGTTAGGT | |
| | dactylus | TTATATTCCGTTGGGGAGATGTAGATATAATGTGTAACTG | |
| | | TCTATTAAATTTAATAGTTATAATTAGTTTTTGATCCTTC | |
| | | TTTGTGGATTAGAAGAATAAGTTACTTTAGGGATAACAGC | |
| | | GTGAT | |
| JQ805 | Macrobrachium | GTGGGACGATAAGACCCTATAAAACTTAATATGAGTTAAG | 1075 |
| 812 | faustinum | CTTGATTTGTGATCTAAGTGAAAAATAGTTTTGTTTGATT | |
| | | TATATTTTGCTGGGGCGGCATAGATATAAAATGTAACTGT | |
| | | CTATTAAATTTAATAGTGTTAATTAGTCTTTGATCCTTCT | |
| | | CTGTGGATTAAAAGAATAAGTTACTTTAGGGATAACAGCG | |
| | | TGAT | |
| AY377 | Macrobrachium | GTGGGACGATAAGACCCTATAAAACTTGATATTATCTAGA | 1076 |
| 842 | heterochirus | TTTGATTTGCGATTTGAGTGAAGAGCGGTTTTATTTATAT | |
| | | GATATTTTGTTGGGGAGACATAGATATAATTAGTAACTGT | |
| | | CTATTAAATTTAATAGTGTTAGTTAGCATTTGATCCTTCT | |
| | | TTGTGGATTAAAAGAATAAGTTACTTTAGGGATAACAGCG | |
| | | TGAT | |
| KP756 | Macrobrachium | GTGGGACGATAAGACCCTATAAAACTTGATATAAGTTTAG | 1077 |
| 688 | scabriculum | CTTGATTTGTGATTTAGGTGAAAGACGATTTTGTTTGGTT | |
| | | TATATTTCGTTGGGGAGATGTAGATATAATGTGTAACTGT | |
| | | CTATTTAATTTAATAAATATAATTAGTATGTGATCCTTCA | |
| | | TTTGTGGATTGGAAGAATAAGTTACTTTAGGGATAACAGC | |
| | | GTGAT | |
| KM101 | Macrobrachium | GTGGGACGATAAGACCCTATAAAACTTGATATAAGCTGGA | 1078 |
| 476 | digueti | CTTGATTTGTGATTTAAGTGAAAAGCAGTTTTGTTTAACT | |
| | | TATATTTTGCTGGGGGGGCATAGATATAAAATGTAACTGT | |
| | | CTGTTAAATTTAATAGCGCTAACTAGTATTTGATCCTTCT | |
| | | TTGTGGATTAAAAGAATAAGTTACTTTAGGGATAACAGCG | |
| | | TGAT | |
| KF383 | Macrobrachium | GGGGGACGATAAGACCCTATAAAACTTAATATCAATTTGG | 1079 |
| 310 | tenellum | TTTGATTTGCGATTTTAGTGAAAAGTAGTCGTGCTTGACT | |
| | | GATATTTCGTTGGGGAGATGAAGATATAAATTGTAACTGT | |
| | | CTATTTAATTCAATAGCTATGGTTAGTGTGTGATCCTTCT | |
| | | TTGTGGATTAAGAGAGTAAGTTACTTTAGGGATAACAGCG | |
| | | TGAT | |
| KP037 | Macrobrachium | GTGGGACGATAAGACCCTATAAAACTTAATATAATTTTGA | 1080 |
| 053 | idae | TTTGATTTGCGATTTTCGGTGAAAAGTAGTCTTGTCGGGT | |
| | | TTATATTTCGTTGGGGAGATGTAGATATAATATGTAACTG | |
| | | TCTATTTAATTTAATAGCTATAACTAGTCTTTGATCCTTC | |
| | | TTTGTGGATTAAAAGAGTAAGTTACTTTAGGGATAACAGC | |
| | | GTGAT | |
| JX025 | Macrobrachium | GGGGGACGATAAGACCCTATAAAGCTTGACAATAACTTCG | 1081 |
| 200 | japonicum | TTATATTATAAATTGTTAGTATAACTTGATTTTAACGGGG | |
| | | GTTTGTTTCGTTGGGGCGACGGGAATATAATAAATAACTG | |
| | | TTCTTTTAAATATAATTACAAAAATGTTTGGTAAATAATT | |
| | | GATCCTCTATTAGAGATTAAAAGATTAAGTTACTTTAGGG | |
| | | ATAACAGCGTAAT | |
| EU493 | Macrobrachium | GTGGGACGATAAGACCCTATAAAACTTTATATAAGTTTAA | 1082 |
| 150 | formosense | TTTGGCTTACAATTTGAGTGAAAAGTAGCTCTATTAAGCT | |
| | | TATGTTTCGTTGGGGAGATGTAGATATAATGTGTAACTGT | |
| | | CTGTTTAATTTAATAATTATAATTAGTGTGTGATCCTTCT | |
| | | TTGTGGATTAAAAGTATAAGTTACTTTAGGGATAACAGCG | |
| | | TGAT | |
| JQ390 | Macrobrachium | GTGGGACGATAAGACCCTATAAAACTTAATATGGGTGTGG | 1083 |
| 474 | dienbienp | CTTGATTTGCGATTAGTAGGTGAAAAGTAATTTTGTTTGG | |
| | huense | CTCATATTTTGTTGGGGAGATGTGGATATAATGTGTAACT | |
| | | GTCTGTTGAATTTAATAGTTATAATTAGTGTATGATCCTT | |
| | | CATTTGTGGATTAGGAGAATAAGTTACTTTAGGGATAACA | |
| | | GCGTGAT | |
| EU493 | Macrobrachium | GGGGGACGATAAGACCCTATAAAACTTTATATAAGTTTGG | 1084 |
| 139 | placidulum | TTTAGTCTATGATTTAGGTAAAAAGTGGTTTTGTCTGGTT | |
| | | TATATTTCGTTGGGGAGATGAGGATATAAAATGTAACTGT | |
| | | CTGTTTAAATTTAATAGTTTTAATTAGTTTGTGATCCCCC | |
| | | TTTGGGGATTAAAAGAGTAAGTTACTTTAGGGATAACAGC | |
| | | GTGAT | |
| JQ362 | Macrobrachium | GTGGGACGATAAGACCCTATAAAACTTTATATAAGAGTAA | 1085 |
| 449 | sintangense | CTTGGTTTATGATTTAGGTGAAAAGTAGCTTTGTTGGGTT | |
| | | TATATTTCGTTGGGGAGATGTAGATATAATATATAACTGT | |
| | | CTGTTTAATTTAATAGCTGTAGTTAGTGTGTGATCCTTCT | |
| | | TTGTGGATTAAAAGTATAAGTTACTTTAGGGATAACAGCG | |
| | | TGAT | |
| JQ359 | Macrobrachium | GTGGGACGATAAGACCCTATAAAACTTAATATAGGTTTGA | 1086 |
| 750 | niphanae | CTTAATTTTATAAAATGGGTGATAAAGTGATTTTGTTTAG | |
| | | TTTATATTTTGTTGGGGCGATATAGATATAATATGTAACT | |
| | | GTCTATTAAATTTAATAATTATAATTAGTTTTTGATCCTA | |
| | | CTTTGTGGATTGAAAGAATAAGTTACTTTAGGGATAACAG | |
| | | CGTGAT | |
| KF383 | Macrobrachium | GTGGGACGATAAGACCCTATAAAACTTGATATAAGTTTAG | 1087 |
| 311 | totonacum | TTTAATTTGTGATTTGAGTGAAGAGCAATTTTGTTAAATT | |
| | | TATATTTTGTTGGGGAGATGTAGATATAAAATATAACTGT | |
| | | CTATTGAATTTAATAGTAATAATTAATTTTTGATCCTTCT | |
| | | NTGAAGATTAGGAGAATAAGTTACTTTAGGGATAACCGCG | |
| | | CTGA | |
| JF491 | Macrobrachium | GTGGGACGATAAGACCCTATAAAACTTGATATAAGTTTAA | 1088 |
| 346 | tuxtlaense | TTTAATTTGTGATTTAAGTGAAGAACAATTTTGTTAAATT | |
| | | TATATTTTGTTGGGGAGATGTAGATATAAAATATAACTGT | |
| | | CTATTAAATTTAATAGTAATAATTAATTTTTGATCCTTCT | |
| | | GTGGGGATTAGGAGAATAAGTTACTTTAGGGATAACAGCG | |
| | | TGAT | |
| KF383 | Macrobrachium | ATGGGACGATAAGACCCTATAAAACTTAATATAAATTTAG | 1089 |
| 313 | vicconi | TTTAATTTGTGATTTAAGTGAAGAGCAGTTTTATTAAATT | |
| | | TATATTTTGTTGGGGAGATGTAGATATAAAGTGTAACTGT | |
| | | CTAATAAATTTAATAATAATAATTAATTTTTGATCCTTCC | |
| | | ATGAGGATTAGGAGAGTAAGTTACTTTAGGGATAACAGCG | |
| | | TGAT | |
| KF383 | Macrobrachium | GTGGGACGATAAGACCCTATAAAACTTGATATAAGTTTAA | 1090 |
| 314 | villalobosi | TTTAATTTGTGATTTAAGTGAAGAGCGATTTTGTTGAATT | |
| | | TATATTTTGTTGGGGAGATGTAGATATAAAGTGTAACTGT | |
| | | CTATTGAATTTAATAGTAATAATTAGTTTTTGATCCTTCT | |
| | | GTGGAGATTAAGAGAGTAAGTTACTTTAGGGATAACAGCG | |
| | | TGAT | |
| KM101 | Macrobrachium | GGGGGACGATAAGACCCTATAAAACTTAATATCTGTTTAG | 1091 |
| 468 | amazonicum | CTTGATTTGTGATTTTAGTGAAAAGTAGTTTTGTTAAGTT | |
| | | GATATTTCGTTGGGGAGATGAAGATATAAATTGTAACTGT | |
| | | CTATTATAATTTGATAATTATAGTTAGTGTGTGATCCTTC | |
| | | TTTGTGGATTAAAAGAATAAGTTACTTTAGGGATAACAGC | |
| | | GTAAT | |
| JF774 | Macrobrachium | AAGGGACGATAAGACCCTATAAAACTTGATATAGTTCAAG | 1092 |
| 072 | canarae | CTTGACTTGTGATTATGAGTAAAAAGTAGTTTTGTTTGGT | |
| | | TTATATTTTGTTGGGGAGATAAAGATATAATGTGTAACTG | |
| | | TCTATTGAATTTTATAGTATTAATTAGAATTTGATCCTTC | |
| | | TTTGAGGATTAAGAGAATAAGTTACTTTAGGGATAACAGC | |
| | | GTGAT | |
| JQ362 | Macrobrachium | GTGGGACGATAAGACCCTATAAAACTTTATATAAAAGTAA | 1093 |
| 452 | tratense | CTTGGTTTATGATTTAGGTGAAAAGTAGCTTTGTTGGGTT | |
| | | TATATTTCGTTGGGGAGATGTAGATATAATATATAACTGT | |
| | | CTGTTTAATTTAATAGCTGTAGTTAGTGTGTGATCCTTCT | |
| | | TTGTGGATTAAAAGTATAAGTTACTTTAGGGATAACAGCG | |
| | | TGAT | |
| JQ362 | Macrobrachium | GTGGGACGATAAGACCCTATAAAACTTAATATGGGTTTGG | 1094 |
| 454 | forcipatum | CTTGATTTGCGATTAGTGGGTGAAAAGTAATTTTGTTTAG | |
| | | CTCATATTTTGTTGGGGAGATGTGGATATAATGTGTAACT | |
| | | GTCTGTTGAATTTAATAGTTATGATTAGTGTGTGATCCTT | |
| | | CATTTGTGGATTAGGAGAATAAGTTACTTTAGGGATAACA | |
| | | GCGTGAT | |
| JQ390 | Macrobrachium | GTGGGACGATAAGACCCTATAAAACTTAATATAGGTTTAG | 1095 |
| 475 | hirsutimanus | CTTGATTTGCAATTATGGGTGAAAAGTGGTTTTGTCTAGG | |
| | | CTTATATTTTGTTGGGGAGATGTAGATATAATGTGTAACT | |
| | | GTCTGTTGAATTTAATAGTTATGATTAGTGTGTGATCCTT | |
| | | CATTTGTGGATTAAGAGAATAAGTTACTTTAGGGATAACA | |
| | | GCGTAAT | |
| GU929 | Macrobrachium | GGGGGACGATAAGACCCTATAAAATTTAATATAAGTTTAA | 1096 |
| 445 | borellii | TTTTATTTGTAATTTAAGTGAAAAATAGTTTAATTAAATT | |
| | | TATATTTCGTTGGGGAGATGTGGATATAAAGTGTAACTGT | |
| | | CTATAAAATTTAATAGTAATTATTAGTAAGTGATCCTTCT | |
| | | TTGTGGATTTGAAGAATAAGTTACTTTAGGGATAACAGCG | |
| | | TAAT | |
| GU929 | Macrobrachium | GGGGGACGATAAGACCCTATAAAACTTGATATAAATTTAG | 1097 |
| 446 | brasiliense | TTTTACTAGTGATTTAAGTGAAAAGTGGTTTAATTAAAGT | |
| | | TATATTTCGTTGGGGAGATGTAAATATAAATGTAACTGTT | |
| | | TATAAAATTAAATAATTATTGTTAGTAGGTGATCCTGCAT | |
| | | ATGCGGATTGAGAAAATAAGTTACTTTAGGGATAACAGCG | |
| | | TAAT | |
| JF310 | Macrobrachium | GTGGGACGATAAGACCCTATAAAACTTGATATAAATTTAG | 1098 |
| 709 | aemulum | TTTGATTTATGATGTGAGTGAAAAGTTGTTTTGCTAGGTT | |
| | | TATATTCCGTTGGGGCGATGAAGATATAAAATGTAACTGT | |
| | | CTATTTAATTTAATAGTATTAATTAGTACTTGATCCTTCT | |
| | | TTGGGGATTAAAAGAATAAGTTACTTTAGGGATAACAGCG | |
| | | TGAT | |
| JF310 | Macrobrachium | GTGGGACGATAAGACCCTATAAAACTTGATATAAGTTTTG | 1099 |
| 717 | handschini | CTTGATTTGTGATTTGTAGTGAAAAGTGGTTTCGTTTAAT | |
| | | TTATATTTCGTTGGGGAGATGTAGATATAATATGTAACTG | |
| | | TCTATTTAATTTAATAGTTATAATTAGTATTTGATCCTTC | |
| | | TTTGTGGATTAAGAGAATAAGTTACTTTAGGGATAACAGC | |
| | | GTGAT | |
| JF310 | Macrobrachium | ATGGGACGATAAGACCCTATAAAACTTAATATAAGTTGAT | 1100 |
| 719 | horstii | CTTGATTTGTGATTTGGGTGAAAAATGATTTTGCTTGGCT | |
| | | TATATTTCGTTGGGGAGATGAAGATATAAAATGTAACTGT | |
| | | CTGTGAAATTTAATAGTGTTAATTAGTTTTTGATCCTTCT | |
| | | TTGTGGATTAAGAGAATAAGTTACTTTAGGGATAACAGCG | |
| | | TGAT | |
| GU929 | Macrobrachium | GGGGGACGATAAGACCCTATAAAACTTAATATAAATTTGA | 1101 |
| 447 | ferreirai | TTTTATTTATAATTTTAGTGAAAAGAAGTTTTAATAAGTT | |
| | | TATATTTTGTTGGGGTGACGTGGATATAAAGTGTAACTGT | |
| | | CCGTAAAATTTAATAATTTTAATTAGTGTCTGATCTTTCT | |
| | | TATGGGGATTAAAAGAGTAAGTTACTTTAGGGATAACAGC | |
| | | GTGAT | |
| DQ194 | Macrobrachium | GTGGGACGATAAGACCCTATAAAACTTGATATGAGTTTAG | 1102 |
| 911 | lanatum | CTTGATTTGTGATTTAGGTGAAAGACGATCTTGTTTGGTT | |
| | | TATATTTCGTTGGGGAGATGTAGATATAATGTGTAACTGT | |
| | | CTATTTAATTTAATAAATATAGTTAGTATGTGATCCTTCA | |
| | | TTTGTGGATTAGAAGAATAAGTTACTTTAGGGATAACAGC | |
| | | GTGAT | |
| JF310 | Macrobrachium | GTGGGACGATAAGACCCTATAAAACTTGATATAAGTCTGA | 1103 |
| 725 | novaeholl | GGGGATTTGTGATTTCAGTAAAAAATAGTCCTATTAGGTA | |
| | andiae | TATATTTCGTTGGGGAGATGTAGATATAATGTGTAACTGT | |
| | | CTTTAAAATTGAATAAATATAGTTAGTTTGTGATCCTGTA | |
| | | TTTACGGATTAAAAGAGTAAGTTACTTTAGGGATAACAGC | |
| | | GTAAT | |
| EF588 | Macrobrachium | ATGGGACGATAAGACCCTATAAAACTTAATATAAGTCTGA | 1104 |
| 319 | tolmerum | TTTAATTTGTGATTTAAGTGAAAATCGATTTTGTTTGACT | |
| | | TATATTTCGTTGGGGAGATGAAGATATAAAGTGTAACTGT | |
| | | CTGTTATAATTTAATAATATTAATTAGTTTTTGATCCTTC | |
| | | TTTGTGGATTAAAAGAATAAGTTACTTTAGGGATAACAGC | |
| | | GTAAT | |
| HM352 | Macrobrachium | AAGGGACGATAAGACCCTATAAAACTTAATATAAATTTTA | 1105 |
| 432 | iheringi | TTTTTTATGTGATTTAAGTGAAACTCAGTTTAGTTTGATT | |
| | | TATATTTCGTTGGGGAGATGTGGATATAAAGTGTAACTGT | |
| | | CTGCTGAATTTAATAATTATTGTTAGTGCGCGATCCTTCT | |
| | | TTGTGGGTTGTAAGAATAAGTTACTTTAGGGATAACAGCG | |
| | | TGAT | |
| JF310 | Macrobrachium | GTGGGACGATAAGACCCTATAAAACTTTATATAAAAGTAG | 1106 |
| 728 | saigonense | TTTGGTTTATAATTTAAGTAAAAAGTAACTTTGCTAAATT | |
| | | TATATTTCGTTGGGGAGATGTAGATATAATGTGTAACTGT | |
| | | CTGTTTAATTTGATAATTGTAGTTAGTATATGATCCTTCT | |
| | | TTGTGGATTAAAAGTATAAGTTACTTTAGGGATAACAGCG | |
| | | TGAT | |
| HM352 | Macrobrachium | GGGGGACGATAAGACCCTATAAAACTTGATATAGATTTAA | 1107 |
| 428 | nattereri | TTTTGCTTGTGATTTCAGTAGAGAGGGGTTTTGATTAGAT | |
| | | CATGTTTCGTTGGGGAGATGTGGATATAAAGTGTAACTGT | |
| | | CTGTAGAATTATATAATTATTATTAGTAGATGATCCTTCT | |
| | | TTGGGGATTAGAAGAATAAGTTACTTTAGGGATAACAGCG | |
| | | TAAT | |
| HM352 | Macrobrachium | GGGGGACGATAAGACCCTATAAAACTTAATATAAATTTAA | 1108 |
| 430 | aracamuni | TTTATTATATGATTTAAGTGAAAAGTAGTTTAAGGTAGTT | |
| | | TATATTTTGTTGGGGTGATATAGATATAAAATGTAACTGT | |
| | | CTGGAAAATTTAATAATTTTAATTAGTGTTTGATCCTTCT | |
| | | TTGTGGATTAAGAGAATAAGTTACTTTAGGGATAACAGCG | |
| | | TGAT | |
| HM352 | Macrobrachium | GGGGGACGATAAGACCCTGTAAAACTTAATATAAATTTGG | 1109 |
| 433 | inpa | TTTTATTTGTGATTTTAGTGAAAAGAAGTTTAAATTAGTT | |
| | | TATATTTTGTTGGGGTGACATAGATATAAAATGTAACTGT | |
| | | CTAAAAAATTTAATAATTTTAATTAGTGTTTGATCCTTCT | |
| | | TTGGGGATTTAAAGAGTAAGTTACTTTAGGGATAACAGCG | |
| | | TAAT | |
| HM352 | Macrobrachium | GGGGGACGATAAGACCCTATAAAACTTTATATAAATTTAA | 1110 |
| 435 | depressimanum | TTTAGTTTATGATATAGGTGAAAAATTGTTTTAATAATTT | |
| | | TATATTTTGTTGGGGAGACATAGATATAATATGTAACTGT | |
| | | TTGTTAAATTTAATAATTATAGTTAATATTTGATCCTTCC | |
| | | TTGAGGATTAAGAGAATAAGTTACTTTAGGGATAACAGCG | |
| | | TAAT | |
| HM352 | Macrobrachium | GTGGGACGATAAGACCCTATAAAACTTGATATAGGTTAAG | 1111 |
| 446 | surinamicum | TTTGATTTGTGATTTAAGTGAAGAGTGGTTTTGCTTGACT | |
| | | TATATTTTGCTGGGGCGGCATGGATATAAAGTGTAACTGT | |
| | | CTATTAAATTTAATAACATTAATTAGTTTCTGATCCTTCT | |
| | | TTGTGGATTAAAAGAATAAGTTACTTTAGGGATAACAGCG | |
| | | TGAT | |
| HM352 | Macrobrachium | GTGGGACGATAAGACCCTATAAAACTTGATATAAGTTGAA | 1112 |
| 462 | denticulatum | CTTGGTTTGTGATTTAGGTGAAAAATAGTCTTGCTTGACT | |
| | | TATATTTCGCTGGGGGGGCATAGATATAAAATGTAACTGT | |
| | | CTATTAAATTTAATAGTGCTAATTAGTCTTTGATCCTTCT | |
| | | CTGTGGATTAAAAGAATAAGTTACTTTAGGGATAACAGCG | |
| | | TGAT | |
| GQ487 | Macrobrachium | GTGGGACGATAAGACCCTATAAAACTTAATATAGGTTTAG | 1113 |
| 497 | pilimanus | TTTGATTCGCGATTGTGGGTGAAAAGTAGTGTTGCTAGTC | |
| | | CTATATTTTGTTGGGGCGATGCGGATAAAATCGGTAACTG | |
| | | TTTGTAAAATTTAATAATTATGATTGATCCTTCGTTTGTG | |
| | | TGGATTAGGAGAATAAGTTACTTTAGGGATAACAGCGTGA | |
| | | T | |
| HM352 | Macrobrachium | GTGGGACGATAAGACCCTATAAAACTTGATATTAGTTTAA | 1114 |
| 464 | ohione | TTTGATTTGCGATTTGAGTGAAGAGCGGTTTTATCTATAT | |
| | | TATATTTTGTTGGGGAGACATAGATATAAACAGTAACTGT | |
| | | CTATTAAATTTAATAGCGTTAGTTAGTACTTGATCCTTCT | |
| | | TTGTGGATTAAAAGAATAAGTTACTTTAGGGATAACAGCG | |
| | | TGAT | |
| EF501 | Macrobrachium | GTGGGACGATAAGACCCTATAAAACTTGATATAAGTTTAA | 1115 |
| 999 | hainanense | TTAACTTATAATTTAAGTGAAAAGTATTTTAAGTGGTTTT | |
| | | ATATTTTGTTGGGGAGATGTAGATATAATGTGTAACTGTT | |
| | | TATAAAATTTAATAATTATAATTAGTATGTGATCCTTCTT | |
| | | TGTGGATTAAAAGTATAAGTTACTTTAGGGATAACAGCGT | |
| | | AAT | |
| EU493 | Macrobrachium | GAGGGACGATAAGACCCTATAAAACTTGACATAATCCAAA | 1116 |
| 138 | lepidactyloides | TATGACTTGAGATTTGAGTGAAGATCGGTCCCGTTTGGCT | |
| | | TGCGTTTCGTTGGGGAGATGAAGATATAAAATGTAACTGT | |
| | | CTGTTTAAATTAAATAGTGTTCAGTAGTTTGTGATCCTTC | |
| | | TCTGTGGATTAAAAGAATAAGTTACTTTAGGGATAACAGC | |
| | | GTGAT | |
| EU493 | Macrobrachium | ATGGGACGATAAGACCCTATAAAACTTAATATGATCCGAC | 1117 |
| 146 | jaroense | CTTGATTTGTGATTTGAGTGAAAAGTGATCTTGTTTGGTT | |
| | | TATATTTCGTTGGGGAGATGAAGATATAAAATGTAACTGT | |
| | | CTATGAAATTTAATAGTGTTAATTAGTTTTTGATCCTTCT | |
| | | TTGTGGATTAAGAGAATAAGTTACTTTAGGGATAACAGCG | |
| | | TGAT | |
| EU493 | Macrobrachium | GTGGGACGATAAGACCCTATAAAACTTGATATAAGTTTTG | 1118 |
| 145 | esculentum | CTTGATTTGTGATTTATGGTGAAAAATGGTTTTGTTTAAC | |
| | | TTATATTTTGTTGGGGAGATGTAGATATAATATGTAACTG | |
| | | TCTATTTAATTTAATAATTATAATTAGTGTTTGATCCTTC | |
| | | TTTGTGGATTAAGAGAATAAGTTACTTTAGGGATAACAGC | |
| | | GTGAT | |
| DQ194 | Macrobrachium | GTGGGACGATAAGACCCTATAAAACTTAATATAAATTTAT | 1119 |
| 910 | maculatum | TTAATTTATGATTTAAGTGAAAAGTAATTTAAGTGGGTTT | |
| | | ATATTTTGTTGGGGAGATGTAGATATAATATGTAACTGTT | |
| | | TATTAAATTTAATAATTATAATTAGTGTGTGATCCTTCTT | |
| | | TGTGGATTAAAAGTATAAGTTACTTTAGGGATAACAGCGT | |
| | | GAT | |
| DQ194 | Macrobrachium | GTGGGACGATAAGACCCTATAAAACTTGATATAAATATAA | 1120 |
| 912 | edentatum | TTTAACTTGTGATTTAGGTAAAAAGTGGTTTTATTGGGTT | |
| | | TATATTTCGTTGGGGAGATGTGGATATAATATGTAACTGT | |
| | | CTGTTTAATTTAATAATTATGATTAGTGTGTGATCCTTCT | |
| | | TTGGGGATTAAGAGTTTAAGTTACTTTAGGGATAACAGCG | |
| | | TGAT | |
| DQ194 | Macrobrachium | GAGGGACGATAAGACCCTATAAAACTTTACATGAATTCAG | 1121 |
| 926 | grandimanus | TTTGATTCTGTGAGCTGATGTGAAAAACAATCCTGCTTAA | |
| | | TTCATGTTTCGTTGGGGAGACGAATATATAATTTGTAACT | |
| | | GTCTGTTTAATTAAATAGTGATAGTTAGTATCTGATCCCT | |
| | | CTATGAGGATTAAAAGAATAAGTTACTTTAGGGATAACAG | |
| | | CGTGAT | |
| DQ194 | Macrobrachium | GAGGGACGATAAGACCCTATAAAACTTAATACAGGTTTGA | 1122 |
| 947 | malayanum | TTAGGTTACGATTTTAGTATAAAGTGGTATTATTAAGTTT | |
| | | GTATTTCGTTGGGGCGATGAAGATATAATTTGTAACTGTC | |
| | | TGTTAAATATAATAGTTATTGCTAGAATTTGATCCTGCTT | |
| | | TGTGGATTATAAGAATAAGTTACTTTAGGGATAACAGCGT | |
| | | AAT | |
| DQ194 | Macrobrachium | ATGGGACGATAAGACCCTATAAAACTTGATATAGGTTTAG | 1123 |
| 949 | meridionale | CTTGGTTTGTGATGTAAGTGAAAAGTTGCTTTGCTTGGCT | |
| | | TATATTTCGTTGGGGAGATGAAGATATAAAGTGTAACTGT | |
| | | CTATTAAATTTAATAGTGCTAATTAGTGTTTGATCCTTCT | |
| | | TTGTGGATTAAAAGAATAAGTTACTTTAGGGATAACAGCG | |
| | | TGAT | |
| DQ194 | Macrobrachium | GTGGGACGATAAGACCCTATAAAACTTAATATAAGTTTTG | 1124 |
| 953 | neglectum | CTTGATTTATGATGTGTGTGAAAAGTAGTTTTGTTTGGTT | |
| | | TATATTTTGTTGGGGAGATATAGATATAATTTGTAACTGT | |
| | | CTATTTAATTTAATAGTTATTGTTAGTGTGTGATCCTTCT | |
| | | TTGTGGATTAAAAGAATAAGTTACTTTAGGGATAACAGCG | |
| | | TGAT | |
| DQ194 | Macrobrachium | GTGGGACGATAAGACCCTATAAAACTTGATATAGGTAAGG | 1125 |
| 954 | platycheles | CTTGGTTTGTAATTGTAGGTTAAAAGTTGTTTTGCTTGGC | |
| | | TTATATTTTGTTGGGGAGATGTAGATATAATGTGTAACTG | |
| | | TCTGTTGAATTTAATAGTTATAATTAGTGTGTGATCCTTC | |
| | | ATTTGTGGATTAGGAGAATAAGTTACTTTAGGGATAACAG | |
| | | CGTGAT | |
| DQ194 | Macrobrachium | GAGGGACGATAAGACCCTATAAAACTTGATATAAATTTAT | 1126 |
| 950 | naso | CTTAATTTGTGATGTGAGTTAAAAGTAATTTTGTTTAAAT | |
| | | TATATTTCGTTGGGGAGATGAAGATATAACATGTAACTGT | |
| | | CTGATTAATTTAATAATCATAATTAGTATTTGATCCTTCT | |
| | | TTGTGGATTTTAAGAAAAAGTTACTTTAGGGATAACAGCG | |
| | | TGAT | |
| DQ194 | Macrobrachium | GGGGGACGATAAGACCCTATAAAACTTTATATAAGTTTGG | 1127 |
| 957 | placidum | TTTAGTCTATGATTTAGGTGAAAAGTGGTTTTGTCTAGTT | |
| | | TATATTTCGTTGGGGAGATGAGGATATAAAATGTAACTGT | |
| | | CTGTTTTAATTTAATAGTTCTAACTAGTTTGTGATCCTTC | |
| | | TTTGGGGATTAAAAGAATAAGTTACTTTAGGGATAACAGC | |
| | | GTGAT | |
| DQ194 | Macrobrachium | ATGGGACGATAAGACCCTATAAAACTTAATATAGGTTTAG | 1128 |
| 960 | yui | TTTAATTTGTAATTTACGTAAAAAGGGGTTTTATTAAATT | |
| | | TATATTTCGTTGGGGAGATGTGGATATAATTTGTAACTGT | |
| | | CTGTAGAATTTAATAATTATAGTTAGAGTTTGATCCTTCG | |
| | | TTGGGGATTAGTAGAATAAGTTACTTTAGGGATAACAGCG | |
| | | TAAT | |
| DQ194 | Macrobrachium | GGGGGACGATAAGACCCTATAAAACTTTATATAAATTTAA | 1129 |
| 961 | shokitai | TTTAACTTGTGAGTTAAGTGAAAAGTAGTTTTGTTGAGTT | |
| | | TATATTTCGTTGGGGAGATGTAGATATAATATGTAACTGT | |
| | | CTATTTAATTTAATAATAATGATTAGTGTGTGATCCTTCT | |
| | | TTGTGGATTAAAAGTTTAAGTTACTTTAGGGATAACAGCG | |
| | | TGAT | |
| AY377 | Macrobrachium | GTGGGACGATAAGACCCTATAAAACTTGATATGAGTGAAG | 1130 |
| 852 | sundaicum | TTTAGTTTGTGGTATAGGTGGAAAGTAATTAAATTTAGTT | |
| | | TATATTTTGTTGGGGTGATGTAGATATAATGTGTAACTGT | |
| | | CTGTTAGAATTTAATAATTATAATTAGTGTTTGATCCTGC | |
| | | TTTGCGGATTAAAAGAATAAGTTACTTTAGGGATAACAGC | |
| | | GTAAT | |
| AY858 | Macrobrachium | GTGGGACGATAAGACCCTATAAAACTTGACACAGACTTAG | 1131 |
| 836 | rude | CTGAATTTGTGATTTTAGTAAAAAGTGATTCTGCGAAGCC | |
| | | TTTGTTTCGTTGGGGAGATGTAGATATAACTTGTAACTGT | |
| | | CTATAAAATTAAATAACTGTGATTAGTTGTTGATCCTTCA | |
| | | CTTGTGGATTAAAAGAATAAGTTACTTTAGGGATAACAGC | |
| | | GTGAT | |
| AY730 | Macrobrachium | AAGGGACGATAAGACCCTATAAAACTTGATATAGTTTGAA | 1132 |
| 051 | lamarrei | CTTAACTTGCGGTTTAAGTGAAAAGTAGTTTTGTTTGTTT | |
| | | TATATTTTGTTGGGGAGATAGAGATATAATGTGTAACTGT | |
| | | CTATTGAATTTTATAGTTTTAATTAGTATTTGATCCTTCT | |
| | | TTGGGGATTAAGAGAGTAAGTTACTTTAGGGATAACAGCG | |
| | | TGAT | |
| AY730 | Macrobrachium | GAGGGACGATAAGACCCTATAAAACTTAATATAGTTTAAA | 1133 |
| 052 | sankolli | TTTAGCTTTCAACTTAAGTAAAAAGTAGTTTTGTTTAGTT | |
| | | TATATTTTGTTGGGGAGATGAAGATATAATGTGTAACTGT | |
| | | CTATTGAATTTTATAGTGTTAATTAGTATTTGATCCTTCT | |
| | | TTGGGGATTAAGAGAATAAGTTACTTTAGGGATAACAGCG | |
| | | TGAT | |
| AY730 | Macrobrachium | GGGGGACGATAAGACCCTATAAAACTTTATATAATCTAAA | 1134 |
| 054 | gangeticum | CTTGACTTGCGATTTAAGTGAAAAGTAGTTTTGTTCGGTT | |
| | | TATATTTCGTTGGGGAGATGAAGATATAATTTGTAACTGT | |
| | | CTATTGAATTTTATAGTATTAAATTAGTATTTGATCCTTC | |
| | | TTTGAGGATTAGGAGAATAAGTTACTTTAGGGATAACAGC | |
| | | GTGAT | |
| KX162 | Trachysal | GGGGGACGATAAGACCCTATAAAGCTTTACAGTTATTTAG | 1135 |
| 763 | ambria | TTGAATTATAAATTGTTAGTATAAAGTTAATTTAACTAAT | |
| | palaestinensis | GTTTGTTTCGTTGGGGTGACGAGAATATAATTTTGTAATT | |
| | | GTTCTTTTATGTTTTATTACAATGATTTTTGGTATAAGAA | |
| | | TGATCCTTTATTAAAGATTAAAAGATTAAGTTACTTTAGG | |
| | | GATAACAGCGTAAT | |
| NC_01 | Euphausia | AAGTGACGATAAGACCCTATAAAACTTGATAAATAAAATT | 1136 |
| 6184 | pacifica | AAAAAAGCGTATTAGTTTATTAAAACTTTTTTTCTGGAAT | |
| | | TTATTTTATTGGGGCGATAAAAATATAAAAAGTAACTGTT | |
| | | TTTAAAATTTTAATGGTTAATAACTAAATTAAATAAATGA | |
| | | TCCTTTAATAAAGATTAAAAGATTAAGTTACTTTAGGGAT | |
| | | AACAGCGTAAT | |
| GQ890 | Euphausia | AAGTGACGATAAGACCCTATAAAACTTAATAAGAAAGATA | 1137 |
| 518 | lucens | AAAAAAGTGCAGTAGTTAATTAAAACTTTTTTTATTAAAC | |
| | | TTATTTTATTGGGGCGATAAAAATGTAAAAAGTAACTGTT | |
| | | TTTAACTTATTAATGATTATAGTCAGAAAGATTGAATGAT | |
| | | CCTTTACTAAAGATTATAAGATTAAGTTACTTTAGGGATA | |
| | | ACAGCGTAAT | |
| MG67 | Euphausia | AAGTGACGATAAGACCCTATAAAACTTAATAAGAAAGATA | 1138 |
| 7874 | vallentini | AAAAAAGTGTAGTAGTTAATTAAAACTTTTTTTGTTAAAC | |
| | | TTATTTTATTGGGGCGATAAAAATGTAAAAAGTAACTGTT | |
| | | TTTAACTTCTTAATGATTATAGTCAGTAAGATTGAATGAT | |
| | | CCTTTAGTAAAGATTATAAGATTAAGTTACTTTAGGGATA | |
| | | ACAGCGTAAT | |
| DQ356 | Euphausia | AAGTGACGATAAGACCCTATAAAACTTGATAAATAAAATT | 1139 |
| 240 | triacantha | AAAAAAGGATATTAGTTAAATTAAAACTTTTTTTGTGGAA | |
| | | TTTATTTTATTGGGGCGATAAAAATATAATTAGTAACTGT | |
| | | TTTTAATATTTTAATGATTAATAATCAGAGTTTAGTAAAT | |
| | | GATCCTCTAATAGAGATTATAAGATTAAGTTACTTTAGGG | |
| | | ATAACAGCGTGAT | |
| AF177 | Euphausia | AAGTGACGATAAGACCCTATAAAACTTGATAAATAAAATT | 1140 |
| 180 | longirostris | AAAAAATGATATTAGTTAATTTAAACTTTTTTTTATGGTA | |
| | | TTTATTTTATTGGGGCGATAAAAATATAATAAGTAACTGT | |
| | | TTTTCATATTTTAATGATAAATAATCAGAGTAAATAAATG | |
| | | ATCCTTTAATAAAGATTGTAAGATTAAGTTACTTTAGGGA | |
| | | TAACAGCGTAAT | |
| AF177 | Euphausia | AAGTGACGATAAGACCCTATAAAACTTTATAAATAAAATA | 1141 |
| 178 | similis | AAAAAAAGATATTAGTTGATTGAAACTTTTTTTTATGGAA | |
| | | TTTATTTTATTGGGGCGATAAAAATATAATAAGTAACTGT | |
| | | TTTTAATATTATAATGATTAATAGTCAAAATATAAATAAA | |
| | | TGATCCTTTAATAAAGATTGCAAGATTAAGTTACTTTAGG | |
| | | GATAACAGCGTAAT | |
| MG67 | Euphausia | AAGTGACGATAAGACCCTATAAAACTTTATAAATTAAACT | 1142 |
| 7872 | recurve | AAAAAAAAATATTAGTTAATTGAAATTTTTTATTGTTGAA | |
| | | TTTATTTTATTGGGGCGATACAAATATAATAAGTAACTGT | |
| | | TTTTCATATTTAAATGATAAATAATCATGAACAATTAAAT | |
| | | GATCCTTTAATAAAGATTGTAAGATTAAGTTACTTTAGGG | |
| | | ATAACAGCGTAAT | |
| MG67 | Euphausia | AAGTGACGATAAGACCCTATAAAACTTTATAAACTAAACT | 1143 |
| 7869 | krohni | AAAAAAAAATATTAGTTAATTGAAATTTTTTATTGTTGAA | |
| | | TTTATTTTATTGGGGCGATTAAAATATAATAAGTAACTGT | |
| | | TTTTTATATTTAAATGATAATTAGTCATGAGTAATTAAAT | |
| | | GATCCTTTAGTAAAGATTGTAAGATTAAGTTACTTTAGGG | |
| | | ATAACAGCGTAAT | |
| DQ356 | Euphausia | AAGTGACGATAAGACCCTATAAAACTTAATAAGGAAAACA | 1144 |
| 239 | frigida | AAAAAAGTGCACTAGTTTATTAAAGCTTCTCTTGTTAAAC | |
| | | TTATTTTATTGGGGCGATAAAAATGTAACAAGTAACTGTT | |
| | | TTTAACTTCTTAATGATTATAGTCAGTAAGATTGAATGAT | |
| | | CCTTTAATAAAGATTATAAGATTAAGTTACTTTAGGGATA | |
| | | ACAGCGTAAT | |
| MG67 | Euphausia | AAGTGACGATAAGACCCTATAAAACTTGATAAATAAAGTT | 1145 |
| 7867 | gibboides | AAAAAAAGATATTAGTTAATTGAAACTTTTTTTTGTGGTG | |
| | | TTTATTTTATTGGGGCGATTAAAATATAATAAGTAACTGT | |
| | | TTTTTACATTTTTATGATAAATAGTCAAGACAAATAAATG | |
| | | ATCCTTTATTAGAGATTATAAGATTAAGTTACTTTAGGGA | |
| | | TAACAGCGTAAT | |
| DQ079 | Euphausia | AGGGGACGATAAGACCCTATAAAACTTTATGCACGACATG | 1146 |
| 713 | eximia | TCCTGTCTTTGAATTATATCCAAATATTGGTTGGCTAAAA | |
| | | GTGCATTCCGTTGGGGAGACGTTGATATAAGCTGTAACTG | |
| | | TCTTATTAAATAGAATAATTATTATTATAATTTGATCCTT | |
| | | CTTTGTGGATAAAAAGAATAAGTTACTTTAGGGATAACAG | |
| | | CGTAAT | |
| MG67 | Euphausia | AAGTGACGATAAGACCCTATAAAACTTTATAAATTAAACT | 1147 |
| 7865 | americana | AAAAAAAAATATTAGTTAATTGAAATTTTTTATTGTTAAA | |
| | | TTTATTTTATTGGGGCGATACAAATATATTAAGTAACTGT | |
| | | TTTTTATATTTAAATGATAAATAATCATAATTAATTAAAT | |
| | | GATCCTCTATTAGAGATTGAAAGATTAAGTTACTTTAGGG | |
| | | ATAACAGCGTAAT | |
| MG67 | Euphausia | AAGTGACGATAAGACCCTATAAAACTTGATAAATAATATT | 1148 |
| 7873 | tenera | AAAAATGAGCATTAGTTAGCTAAATCTTTTTTTGTAAAGT | |
| | | TTATTTTATTGGGGCGATAAAAATATAAATAGTAACTGTT | |
| | | TTTAAAATTTTAATGGTAGTAAACAAAAATATTAAATGAT | |
| | | CCTTTAATAAAGATTATAAGATTAAGTTACTTTAGGGATA | |
| | | ACAGCGTAAT | |
| MG67 | Euphausia | AAGTGACGATAAGACCCTATAAAGCTTAATAAACAAAACT | 1149 |
| 7871 | pseudogibba | AAAAATAAATATTAGTTAATTAAATTTTTTTTTGTGTAGT | |
| | | TTATTTTATTGGGGCGATAAAAATATAAATAGTAACTGTT | |
| | | TTTAATATTTTAATGATAAATAATCAAAAATTAAATGATC | |
| | | CTTTAATAAAGATTATAAGATTAAGCTACTTTAGGGATAA | |
| | | CAGCGTAAT | |
| MG67 | Euphausia | AAGTGACGATAAGACCCTATAAAACTTGATAAATAAAATT | 1150 |
| 7868 | hemigibba | AAAAATAAATATTAGTTAATTAAATTTTTTTTTGTGAAGT | |
| | | TTATTTTATTGGGGCGATAAAAATATAAATAGTAACTGTT | |
| | | TTTAATATTTTAATGATAATAGTCAAAAATTAAATGATCC | |
| | | TTTAATAAAGATTATAAGATTAAGTTACTTTAGGGATAAC | |
| | | AGCGTGAT | |
| MG67 | Euphausia | AAGTGACGATAAGACCCTATAAAACTTTATAAATTTAAAT | 1151 |
| 7866 | brevis | TAAAAAAAGTATTAGTAGTTTAAAATTTTTTATTGTTAAA | |
| | | TTTATTTTATTGGGGCGATACAAATATAATAAATAACTGT | |
| | | TTATATATTTAAATGACGAATAATCATGAATAATTAAATG | |
| | | ATCCTTTAATAAAGATTGTAAGATTAAGTTACTTTAGGGA | |
| | | TAACAGCGTAAT | |
| KF182 | Hymenopenaeus | AAGGGACGATAAGACCCTATAAAGCTTTACAATAAATAAA | 1152 |
| 582 | debilis | TTAAATTATAAATTATTAGTTTAACTTAATTTAATTTGTG | |
| | | TTTGTTTCGTTGGGGCGACGAGAATATAATAATAACTGTT | |
| | | CTTTTATAAAATTAACAAAATTAATTGGATAATAATTGAT | |
| | | CCTTTAATAAAGATTAAAAGATTAAGTTACTTTAGGGATA | |
| | | ACAGCGTAAT | |
| KC515 | Nematopalaemon | GTGGGACGATAAGACCCTATAAAACTTTATATAAATGGAT | 1153 |
| 042 | tenuipes | TGAGCCTGTGATTTATCTGGTAAAGCGGGCTGGGTTTTTT | |
| | | TATATTTTGTTGGGGCGACATTGATAAAATTTGTTGGTAA | |
| | | CTGTTCTGTGAATTTTATAATGGTGGTTAGGTAAAGATCC | |
| | | TTTTATAGGGATTAAGAGTTTAAGTTACTTTAGGGATAAC | |
| | | AGCGTAAT | |

### Primer Systems

Primers were designed manually on a multiple DNA sequence alignment of the mitochondrial 16S rDNA of approximately 90 bivalve species using the CLC Genomics Workbench 10.1.1. The designed primers were checked for their physical and structural properties (e.g. formation of dimers, secondary structure, annealing temperature) using Oligo Calc, the OligoAnalyzer Tool provided by Integrated DNA Technologies (IDT) and the online product descriptions from TIB Molbiol (Berlin, Germany). The primers, listed in Table 5, were synthesized by TIB Molbiol. Table 5 also shows the Illumina overhang adapter sequences which were linked to the target-specific primers.

TABLE 5

| | | SEQ ID |
|---|---|---|
| Name | Sequence 5′ → 3′ | NO: |
| mussel | | |
| For_Mu | CCTTTTGCATAAGGGTTTTTCAAG | 14 |
| Rev1_Mu | CGAATAGTATCTAGCCGCCATTC | 24 |
| Rev2_Mu | GCAAATAGCATATCACTTTCACCTC | 25 |
| scallop | | |
| For_Mu | TGCTAAGGTAGCTAAATTATGGCC | 13 |
| Rev_Mu | CTTCACGGGGTCTTCTCGTC | 23 |
| oyster | | |
| For_Mu | GGTAGCGAAATTCCTTGCCTT | 12 |
| Rev_Mu | AAAGTTGCACGGGGTCTT | 22 |
| overhang | | |
| Forward | TCGTCGGCAGCGTCAGATGTG | 26 |
| | TATAAGAGACAG | |
| Reverse | GTCTCGTGGGCTCGGAGATGT | 27 |
| | GTATAAGAGACAG | |

All in-house designed primers were tested in real-time PCR with DNA extracted from the eleven reference samples, comprising three mussel, six scallop, and two oyster species. During optimization, the following PCR conditions/parameters were kept constant and applied as published previously: DNA input amount of 12.5 ng, ‘ready-to-use’ HotStarTaq Master Mix Kit, annealing temperature (62° C.), 25 cycles (Dobrovolny, S. et al., 2019). Only one variable, the addition of magnesium chloride solution, was modified (addition of 1.5 mM or 3 mM MgCl2). Real-time PCR reactions were carried out using a fluorescent intercalating dye (EvaGreen® (20× in water)) in strip tubes or in 96-well plates, depending on the thermocycler used, the Rotor-Gene Q (Qiagen) or the LightCycler® 480 System (Roche, Penzberg, Germany), respectively. The total volume of the PCR reactions was 25 μL, consisting of 22.5 μL reaction mix and 2.5 μL of template DNA (diluted DNA samples (5 ng/μL)) or water as negative control. In the reaction mix, the HotStarTaq Master Mix Kit (Qiagen) was used at a final concentration of 1× and the final concentration of primers was 0.2 μM, expect the forward primer for mussels (0.4 μM). PCR cycling conditions were 15 min initial denaturation at 95° C., 25 cycles at 95° C., 62° C. and 72° C. for 30 s each, and a final elongation for 10 min at 72° C. The primer pairs for mussels, scallop and oysters with and without Illumina overhang adapter sequences were first used in singleplex PCR assays. Then, the seven primers (three forward and four reverse primers) listed in Table 5 were combined in a triplex assay. The identity of the PCR products was confirmed by melting curve analysis and/or agarose gel electrophoresis.

### Library Preparation and NGS

In general, samples were sequenced by using either the MiSeq® or the iSeq® platform (Illumina, San Diego, California, USA). DNA extracts were diluted to a DNA concentration of 5 ng/μL. Extracts with a DNA concentration <5 ng/μL were used undiluted.

DNA library preparation was performed according to Dobrovolny, S. et al. (2019) with minor modifications (excess of magnesium chloride, final concentration 3 mM; average library size: 278 bp; diluted libraries of the iSeq© system were denatured automatically on the instrument).

For the MiSeq© and iSeq® platform, the DNA library was adjusted to 4 nM and 1 nM, respectively, with 10 mM Tris-HCL, pH 8.6. After pooling individual DNA libraries (5 μL MiSeq®, 7 μL iSeq®), the DNA concentration was determined using Qubit© 2.0 fluorimeter.

All sequencing runs were performed using either the MiSeq® Reagent Kit v2 (300-cycles) or the iSeq® 100 i1 Reagent v2 (300-cycles) with a final loading concentration of 8 μM. The pooled DNA libraries contained a 5% PhiX spike-in.

Reference samples were sequenced in six replicates (three sequencing runs, two replicates per run), while DNA extract mixtures were sequenced in nine replicates (three sequencing runs, three replicates per run). Commercial food products (O5-Mi86, above the double line in Table 9) were sequenced in one replicate (three sequencing runs, one replicate per run) and food products (O1-S61, below the double line in Table 9) were sequenced at least once by using either the MiSeq® or the iSeq® platform.

### NGS Data Analysis Using Galaxy

After paired-end sequencing, the resulting FastQ files, generated by the instrument control software, were used as input for data analysis. The sequencing output in FastQ format was then processed with an analysis pipeline as described previously by using Galaxy (Version 19.01) (Dobrovolny, S. et al., 2019). The published amplicon analysis workflow was modified as follows: the target-specific primers were trimmed from both ends using the tool Cutadapt and reads were not clustered into Operational Taxonomic Units (OTUs) (Martin, M., 2011). Completely identical sequences were collapsed into a single representative sequence with the tool Dereplicate to minimize the number of reads, and then compared against a customized database for bivalves using BLASTn (Edgar, R. C., 2010).

### Results

### Barcode Region and Primer Systems

The aim was to develop a DNA metabarcoding method allowing the differentiation between species belonging to the bivalves Pectinidae, Ostreidae, and Mytilidae. To be applicable in routine analysis, the method should allow identifying the economically most important bivalve species in raw and highly processed food products.

Three primer sets were designed, one for each of the three bivalves, Pectinidae, Ostreidae, and Mytilidae. Primer pairs consisting of one forward and one reverse primer allowed amplifying the DNA barcode region in scallop and oyster species (Table 5). However, in case of mussels, a primer set consisting of one forward primer and two reverse primers (Table 5) was necessary to obtain a PCR product for the mussel species listed in Table 3. With the three primer sets, PCR products differing in at least one base should be obtained for all bivalve species of interest.

Further sequence alignments indicated that the DNA barcode region selected does not allow distinguishing between all species of the following genera: Chlamys spp., Euvola spp., Pecten spp., Crassostrea spp., Magallana spp., Ostrea spp. and Saccostrea spp. These species cannot be distinguished: Chlamys rubida and Chlamys behringiana; Pecten albicans, Pecten fumatus, Pecten jacobaeus, Pecten keppelianus, Pecten novaezelandiae, Pecten sucicostatus, Crassostrea hongkongensis and Crassostrea rivularis; Ostrea angelica and Ostrea lurida; as well as Ostrea permollis and Ostrea puelchana; and Saccostrea echinata, Saccostrea glomerata and Saccostrea mytiloides. In addition, two mussel species, Mytilus platensis and Mytilus chilensis, can also not be distinguished (for Mytilus platensis only one DNA sequence entry was in the public databases provided by NCBI). However, differentiation at the genus level (Chlamys spp., Pecten spp., Crassostrea spp. Ostrea spp., Mytilus spp.) is sufficient according to the “Codex Alimentarius Austriacus” chapter B35 (see BMGF-75210/0026-II/B/13/2017, 2007).

When the primers were tested in singleplex PCR assays, for each of the reference samples a PCR product of about 150 bp in length was obtained by increasing the concentration of the forward primer for mussels to 0.4 μM and keeping the concentration of the other six primers at 0.2 μM. In addition, it was tested whether the seven primers could be combined to a triplex system. PCR products for the bivalve species of interest were obtained in one and the same vial by increasing the magnesium chloride concentration to a final concentration of 3 mM. Thus, it was achieved to perform the triplex PCR assay.

### Library Preparation, Pooling of Libraries and Sequencing

Library preparation, pooling of 5 μL or 7 μL per normalized DNA library and the sequencing process were performed as described previously (Dobrovolny, S. et al., 2019). Sequencing runs were performed in triplicates and the average run metrics were as follows: cluster density (969 K/mm²) on the flow cell, cluster passing filter (70.22%) as well as the Q-scores (Q30) for read1 and read2 were 92.6% and 89.28%, respectively. 5.02% of the total reads were identified as PhiX control sequences with an error rate of 1.49%.

Analysis of DNA Extracts from Reference Samples

PCR products were obtained for each of the reference samples, comprising three mussel samples (M12, M13 and M27, Table 2), six scallop samples (S42, S46, S47, S49, S50, and S55, Table 2) and two oyster samples (O2 and O3, Table 2). Sequencing results for reference samples are summarized in Table 6.

Table 6 shows results for DNA extracts from reference samples. The numbers are mean values (n=6, three sequencing runs, 2 replicates per run). The table shows the mean values of the total number of raw reads, the total number of reads that passed the analysis pipeline in Galaxy as well as the total number and percentage of reads that were assigned correctly to the eleven species (based on 6 replicates).

No significant differences were observed in the total number of reads (before data analysis process) between these species, except Mytilus galloprovincialis (162843), Perna canaliculus (169631), and Mytilus edulis (134500). With the exception of Perna canaliculus, >70% of the reads passed the amplicon analysis workflow. All three mussel species, six scallop species and two oyster species could be identified with this workflow at high rate (>97.5%), except Mytilus edulis.

TABLE 6

| | | | | Total | | |
|---|---|---|---|---|---|---|
| | Declaration on | | Total | number of | Number | Percentage |
| | the product: | | number | reads | of reads | of reads |
| Sample | Scientific/Latin | Species | of raw | passing the | assigned | assigned |
| ID | name | identified¹ | reads | workflow | correctly | correctly % |
| O2 | Ostrea edulis | Ostrea edulis | 78559 | 63491 | 61875 | 97.46 |
| O3 | Crassostrea | Magallana | 76143 | 65389 | 64125 | 98.07 |
| | gigas | gigas | | | | |
| M12 | Mytilus | Mytilus | 162843 | 150678 | 149315 | 99.09 |
| | galloprovincialis | galloprovincialis | | | | |
| M13 | Perna | Perna | 169631 | 104861 | 103350 | 98.56 |
| | canaliculus | canaliculus | | | | |
| M27 | Mytilus edulis | Mytilus edulis | 134500 | 120686 | 105024 | 87.02 |
| S42 | Mizuhopecten | Mizuhopecten | 75927 | 58069 | 57058 | 98.26 |
| | yessoensis | yessoensis | | | | |
| S46 | Pecten | Pecten spp. | 79472 | 61484 | 60514 | 98.42 |
| | jacobaeus | | | | | |
| S47 | Zygochlamys | Zygochlamys | 77747 | 59245 | 58429 | 98.62 |
| | patagonica | patagonica | | | | |
| S49 | Placopecten | Placopecten | 79131 | 61531 | 60886 | 98.95 |
| | magellanicus | magellanicus | | | | |
| S50 | Argopecten | Argopecten | 77383 | 55455 | 54588 | 98.44 |
| | purpuratus | purpuratus | | | | |
| S55 | Aequipecten | Aequipecten | 79141 | 56064 | 55800 | 99.53 |
| | opercularis | opercularis | | | | |

### Analysis of DNA Extract Mixtures

Six ternary DNA extract mixtures were analysed containing the DNA of the three bivalve species Pectinidae, Ostreidae, and Mytilidae in ratios of 98.0:1.5:0.5 (v/v/v). The composition of the DNA extract mixtures and the results obtained by DNA metabarcoding are summarized in Table 7. Table 7 shows the results for ternary DNA extract mixtures representing the three bivalve species of interest. DNA extracts (5 ng/μL) were mixed in a ratio of 98.0:1.5:0.5 (v/v/v). Numbers are mean values (n=9, three sequencing runs, 3 replicates per run). The total number of raw reads ranged from 80856 to 147443 and the reads that passed the workflow were in the range from 65961 to 147196. For the main components (98.0%), the number of reads assigned correctly ranged from 62434 to 140147. In addition, both minor components (1.5% and 0.5%) could be identified. The number of reads assigned correctly was in the range from 1710 to 4356 and 555 to 1478, respectively.

TABLE 7

| | | | | Total | | | | | | |
|---|---|---|---|---|---|---|---|---|---|---|
| | | | Total | number | | | | | | |
| Proportion | | | number | of reads | Reads assigned correctly | | | | | |
| Species 1 | Species 2 | Species 3 | of raw | passing the | Species | | Species | | Species | |
| (98%) | (1.5%) | (0.5%) | reads | workflow | 1 | (%) | 2 | (%) | 3 | (%) |
| Crassostrea | Mytilus | Pecten spp. | 80856 | 69506 | 66430 | 95.57 | 1985 | 2.86 | 658 | 0.95 |
| gigas | galloprovincialis | | | | | | | | | |
| Crassostrea | Pecten spp. | Mytilus | 89552 | 76669 | 73114 | 95.36 | 2182 | 2.85 | 894 | 1.17 |
| gigas | | galloprovincialis | | | | | | | | |
| Pecten spp. | Crassostrea | Mytilus | 88971 | 69682 | 66291 | 95.13 | 1710 | 2.45 | 922 | 1.32 |
| | gigas | galloprovincialis | | | | | | | | |
| Pecten spp. | Mytilus | Crassostrea | 84085 | 65961 | 62434 | 94.65 | 2281 | 3.46 | 555 | 0.84 |
| | galloprovincialis | gigas | | | | | | | | |
| Mytilus | Pecten spp. | Crassostrea | 159737 | 147196 | 140147 | 95.21 | 4356 | 2.96 | 1478 | 1.00 |
| galloprovincialis | | gigas | | | | | | | | |
| Mytilus | Crassostrea | Pecten spp. | 147443 | 136629 | 130986 | 95.87 | 3304 | 2.42 | 1156 | 0.85 |
| galloprovincialis | gigas | | | | | | | | | |

In addition, three DNA extract mixtures containing DNA from species belonging to one bivalve species were analysed (Table 8). Table 8 shows the results for DNA extract mixtures representing one bivalve species. DNA from minor components was present in a proportion of 1% each. In addition, results for a DNA extract mixture containing DNA from a squid species (Sepiella inermis) as main component (97.0%) and DNA from three bivalve species (1% each) is shown. Numbers are mean values (n=9, three sequencing runs, 3 replicates per run). The mixtures contained DNA from a scallop or mussel species, respectively. DNA from other bivalve species was present in a proportion of 1.0% each. Both species being present as main components, Placopecten magellanicus and Perna canaliculus, could be identified, with the number of reads assigned correctly ranging from 58156 to 77483. However, quite different numbers of reads were correctly assigned to the minor components, ranging from 626 (Mizuhopecten yessoensis) to 50391 (Mytilus galloprovincialis).

A further DNA extract mixture was analysed containing DNA from the squid species Sepiella inermis as main component (97.0%) and DNA from the bivalve species Placopecten magellanicus, Ostrea edulis and Perna canaliculus as minor components (1.0% each). As expected, in this mixture, the main component could not be detected because the primers are not suitable for amplification of the target region for Sepiella inermis. 31424, 28162, and 806 reads, respectively, were assigned correctly to the three bivalve species.

TABLE 8

| | | Total | Total number | | |
|---|---|---|---|---|---|
| | Minor | number | of reads | Reads | Percentage of |
| Main | component | of raw | passed the | assigned | reads assigned |
| component | (1.0% each) | reads | workflow | correctly | correctly (%) |
| Placopecten | | 83526* | 65446 | 58156 | 88.86 |
| magellanicus | | | | | |
| | Mizuhopecten | | | 626 | 0.96 |
| | yessoensis | | | | |
| | Pecten spp. | | | 817 | 1.25 |
| | Zygochlamys | | | 4534 | 6.93 |
| | patagonica | | | | |
| | Argopecten | | | 663 | 1.01 |
| | purpuratus | | | | |
| | Aequipecten | | | 35 | 0.05 |
| | opercularis | | | | |
| Placopecten | | 84282* | 66691 | 63628 | 95.41 |
| magellanicus | | | | | |
| | Magallana gigas | | | 1298 | 1.95 |
| | Ostrea edulis | | | 1088 | 1.63 |
| Perna | | 179227* | 128882 | 77483 | 60.12 |
| canaliculus | | | | | |
| | Mytilus | | | 50391 | 39.10 |
| | galloprovincialis | | | | |
| | Mytilus edulis | | | 824 | 0.64 |
| Sepiella inermis | | 78467 | 61415 | | |
| | Placopecten | | | 31424 | 51.17 |
| | magellanicus | | | | |
| | Ostrea edulis | | | 28162 | 45.86 |
| | Perna canaliculus | | | 806 | 1.31 |

*Number of values (n = 6, three sequencing runs, 2 replicates per run)

### Analysis of Commercial Seafood Samples

In order to investigate the applicability of the DNA metabarcoding method to foodstuffs, DNA extracts from 75 commercial food products were analysed. According to declaration, eight samples (O1 and O4-O10) contained oyster species, 27 samples (M11, M14-M26, and M28-M40) mussel species, 15 samples (S41, S43-45, S48, S51-S55, and S56-S61) scallop species and 25 samples (Mi62-Mi86) were mixed-species seafood products (Table 9). Table 9 shows the results obtained for commercial seafood samples. Samples listed above the double line were sequenced with the MiSeq® (three sequencing runs, 1 replicate per run, numbers are mean values); samples listed below the double line were sequenced either with the MiSeq® or the iSeq®. The ingredient list of 30 out of 75 food products did not give any information on the bivalve species. 39 samples were declared to contain “Crassostrea gigas”, “Mytilus galloprovincialis”, “Mytilus chilensis”, “Mytilus edulis”, “Zygochlamys patagonica”, “Chlamys opercularis”, “Placopecten magellanicus”, “Pecten maximus”, or “Patinopecten yessoensis”. The remaining samples (n=6) were labelled with “Mytilus spp.” and “Pecten spp.”.

Three oyster species (Saccostrea malabonensis, Magallana bilineata, Magallana gigas), three mussel species (Mytilus galloprovincialis, Mytilus edulis, Perna canaliculus), and three scallop species (Aequipecten opercularis, Placopecten magellanicus, Pecten spp.) were detected in food products (O4, O8, M17, M19, M23, M25, M26, M28, M31, M32, M35, M38-M40, S51, S56, S58-S60, Mi63, Mi65, Mi70, Mi71, Mi73-Mi76, Mi81, Mi83, Mi85, and Mi86) although they were not declared on the label. In four (O1 and O5-O7) out of eight oyster products declared to contain “Crassostrea gigas”, this species was identified.

In 21 products (M11, M16, M18, M21, M24, M33-M35, M37, M39, M40, Mi62, Mi64, Mi66, Mi69, Mi72, Mi77-Mi80, and Mi84), the mussel species Mytilus galloprovincialis was detected, indicating that it is one of the most commonly used bivalve species. In addition to Mytilus galloprovincialis, Mytilus edulis was identified (percentage of reads assigned correctly >1%) in 13 products (M24, M33, M34, M39, Mi62, Mi64, Mi66, Mi69, Mi72, Mi78-Mi80, and Mi84). However, in none of the products declared to contain Mytilus chilensis, Mytilus chilensis was detected. In four products, Mytilus edulis could not be detected although it was declared on the label.

Placopecten magellanicus and Patinopecten yessoensis were listed as ingredients in samples S41, S45, S54, and S57 and samples S48, S52, and S61, respectively. The results confirmed the presence of these two species, except for sample S57. In sample S43, declared to contain Pecten maximus, the species Mizuhopecten yessoensis was detected. In sample S44 and S53, declared as Pecten spp., the species Mizuhopecten yessoensis was also analysed.

Thus, the method of the invention allows to specifically control food declaration.

TABLE 9

| | | | | | Total | | |
|---|---|---|---|---|---|---|---|
| | Declaration on the product | | | | number of | | Percentage |
| | | Product | | Total | reads | Reads | of reads |
| Sample | | description | | number of | passed the | assigned | assigned |
| ID | Scientific/Latin name | [Eng] | Species identified | raw reads | workflow | correctly | correctly % |
| O5 | Crassostrea gigas | Oyster in sunflower oil | Magallana gigas | 76930¹ | 65728 | 64369 | 97.93 |
| O6 | Crassostrea gigas | Oyster in sunflower oil | Magallana gigas | 44848¹ | 38547 | 37610 | 97.57 |
| O7 | Crassostrea gigas | Oyster in water | Magallana gigas | 76247 | 64917 | 63700 | 98.13 |
| O8 | not declared | Oyster sauce | Saccostrea malabonensis | 14470 | 11658 | 5442 | 46.68 |
| | | | Magallana bilineata | | | 4652 | 39.91 |
| M23 | not declared | Mussel with sherry | Mytilus galloprovincialis | 33517 | 30794 | 30358 | 98.58 |
| | | vinegar | | | | | |
| M25 | not declared | Mussel in marinade | Mytilus galloprovincialis | 163188 | 151688 | 150700 | 99.35 |
| | | sauce | | | | | |
| M26 | not declared | Grilled blue mussel | Mytilus galloprovincialis | 163106 | 151608 | 150433 | 99.23 |
| M29 | Mytilus galloprovincialis | Blue mussel in | Mytilus galloprovincialis | 153435 | 140475 | 132354 | 94.22 |
| | | tomato sauce | Mytilus edulis | | | 7937 | 5.65 |
| M30 | Mytilus galloprovincialis | Blue mussel | Mytilus galloprovincialis | 185479 | 171890 | 170624 | 99.26 |
| | | A la mariniere | Mytilus edulis | | | 1156 | 0.67 |
| M31 | not declared | Blue mussel in | Mytilus galloprovincialis | 170303 | 158379 | 157015 | 99.14 |
| | | organic marinade | Mytilus edulis | | | 1267 | 0.80 |
| M32 | not declared | Marinated blue | Mytilus galloprovincialis | 159181 | 144788 | 143399 | 99.04 |
| | | mussel | Mytilus edulis | | | 1308 | 0.90 |
| M33 | Mytilus chilensis | Mussel in | Mytilus galloprovincialis | 167903 | 151219 | 118879 | 78.61 |
| | | Escabeche | Mytilus edulis | | | 31737 | 20.99 |
| M34 | Mytilus chilensis | Mussel | Mytilus galloprovincialis | 152112 | 138768 | 87964 | 63.39 |
| | | | Mytilus edulis | | | 49601 | 35.74 |
| M36 | Mytilus galloprovincialis | Blue mussel | Mytilus galloprovincialis | 176963 | 163721 | 162224 | 99.09 |
| | | marinated | Mytilus edulis | | | 1323 | 0.81 |
| M37 | Mytilus edulis | Mussel in honey | Mytilus galloprovincialis | 149364 | 136868 | 135249 | 98.82 |
| | | mustard sauce | Mytilus edulis | | | 1400 | 1.02 |
| M38 | not declared | Blue mussel | Mytilus galloprovincialis | 138801 | 127244 | 125980 | 99.01 |
| | | in marinade | Mytilus edulis | | | 1056 | 0.83 |
| S58 | not declared | Rillettes de | Aequipecten opercularis | 62787 | 44307 | 42716 | 96.41 |
| | | Saint-Jacques | Mytilus galloprovincialis | | | 1330 | 3.00 |
| S59 | not declared | Small scallop in | Aequipecten opercularis | 82550 | 59722 | 58296 | 97.61 |
| | | galician sauce | | | | | |
| Mi62 | Mytilus chilensis | Seafood mix | Mytilus galloprovincialis | 618324 | 569815 | 433439 | 76.07 |
| | | | Mytilus edulis | | | 134543 | 23.61 |
| Mi63 | not declared | Sauce with | Mytilus edulis | 152170 | 139306 | 73550 | 52.80 |
| | | seafood | Mytilus galloprovincialis | | | 64729 | 46.47 |
| Mi64 | Mytilus chilensis, | Seafood mix | Mytilus galloprovincialis | 131285 | 119350 | 81590 | 68.36 |
| | Mytilus edulis | | Mytilus edulis | | | 37211 | 31.18 |
| Mi65 | not declared | Bouillabaisse | Mytilus galloprovincialis | 157311 | 143479 | 138535 | 96.55 |
| | | Marseille | Mytilus edulis | | | 4777 | 3.33 |
| Mi66 | Mytilus chilensis | Seafood mix | Mytilus galloprovincialis | 152535 | 140047 | 92024 | 65.71 |
| | | | Mytilus edulis | | | 47415 | 33.86 |
| Mi67 | Mytilus spp. | Seafood mix | Mytilus galloprovincialis | 76544 | 69081 | 48275 | 69.88 |
| | | | Mytilus edulis | | | 20459 | 29.62 |
| Mi68 | Mytilus galloprovincialis | Sea fruit salad in | Mytilus galloprovincialis | 157861 | 145671 | 144468 | 99.17 |
| | | sunflower oil | Mytilus edulis | | | 1046 | 0.72 |
| Mi69 | Mytilus chilensis | Seafood mix | Mytilus galloprovincialis | 140227 | 128007 | 85679 | 66.93 |
| | | | Mytilus edulis | | | 41686 | 32.57 |
| Mi70 | not declared | Sea fruit salad | Mytilus galloprovincialis | 120677 | 106674 | 101121 | 94.80 |
| | | fantasy | Mytilus edulis | | | 5413 | 5.07 |
| Mi71 | not declared | Seafood mix | Mytilus galloprovincialis | 160546 | 147278 | 79680 | 54.10 |
| | | | Mytilus edulis | | | 66675 | 45.27 |
| Mi72 | Mytilus chilensis | Seafood mix | Mytilus galloprovincialis | 160059 | 146539 | 91557 | 62.48 |
| | | | Mytilus edulis | | | 54271 | 37.03 |
| Mi73 | not declared | Seafood mix | Mytilus edulis | 150500 | 137634 | 78942 | 57.36 |
| | | | Mytilus galloprovincialis | | | 57608 | 41.86 |
| Mi74 | not declared | Seafood mix | Mytilus galloprovincialis | 168841 | 155701 | 79035 | 50.76 |
| | | | Mytilus edulis | | | 75612 | 48.56 |
| Mi75 | not declared | Pizza Frutti di | Mytilus galloprovincialis | 181822¹ | 172620 | 95184 | 55.14 |
| | | mare | Mytilus edulis | | | 71440 | 41.39 |
| Mi76 | not declared | Paella | Mytilus galloprovincialis | 150431 | 139511 | 138335 | 99.16 |
| | | | Mytilus edulis | | | 1070 | 0.77 |
| Mi77 | Mytilus edulis, | Paella | Mytilus galloprovincialis | 141816 | 132092 | 130768 | 99.00 |
| | Mytilus chilensis | | Mytilus edulis | | | 1242 | 0.94 |
| Mi78 | Mytilus chilensis | Seafood all'Olio | Mytilus galloprovincialis | 134717 | 122906 | 73482 | 59.79 |
| | | | Mytilus edulis | | | 48774 | 39.68 |
| Mi79 | Mytilus chilensis | Seafood mix | Mytilus galloprovincialis | 148773 | 137122 | 73035 | 53.26 |
| | | | Mytilus edulis | | | 63249 | 46.13 |
| Mi80 | Mytilus chilensis | Seafood mix | Mytilus galloprovincialis | 136695 | 126608 | 88130 | 69.61 |
| | | | Mytilus edulis | | | 37970 | 29.99 |
| Mi81 | not declared | Sea fruit salad | Mytilus galloprovincialis | 153499 | 142736 | 141578 | 99.19 |
| | | | Mytilus edulis | | | 1022 | 0.72 |
| Mi82 | Zygochlamys patagonica, | Scallop terrine | Zygochlamys patagonica | 76554 | 59181 | 57329 | 96.87 |
| | Chlamys opercularis | | | | | | |
| Mi83 | not declared | Terrine of salmon | Pecten spp. | 64675¹ | 51387 | 75476 | 146.88 |
| | | and great scallop | | | | | |
| Mi84 | Mytilus chilensis | Seafood mix | Mytilus galloprovincialis | 163885 | 150852 | 124468 | 82.51 |
| | | | Mytilus edulis | | | 25916 | 17.18 |
| Mi85 | not declared | Instant noodle | Mytilus galloprovincialis | 15409 | 14118 | 13750 | 97.39 |
| | | seafood, mild | | | | | |
| Mi86 | not declared | Instant noodle | Mytilus galloprovincialis | 9787 | 8892 | 8473 | 95.29 |
| | | seafood, spicy | | | | | |
| O1 | Crassostrea gigas | Oyster | Magallana gigas | 139319² | 134073 | 133493 | 99.57 |
| O4 | not declared | Oyster | Magallana gigas | 46089² | 40991 | 40279 | 98.26 |
| O9 | not declared | Oyster sauce | | not evaluable³ | | | |
| O10 | not declared | Oyster sauce | | not evaluable³ | | | |
| M11 | Mytilus edulis | Mussel | Mytilus galloprovincialis | 23766² | 22546 | 22147 | 98.23 |
| M14 | Mytilus spp. | Blue mussel | Mytilus galloprovincialis | 126880² | 119717 | 79522 | 66.42 |
| M15 | Mytilus spp. | Blue mussel | Mytilus edulis | 126880² | 119717 | 39555 | 33.04 |
| | | | Mytilus galloprovincialis | | | 220226 | 99.79 |
| M16 | Mytilus edulis | Bouchot mussel | Mytilus galloprovincialis | 51292² | 49604 | 48832 | 98.44 |
| M17 | not declared | Grilled blue | Mytilus galloprovincialis | 9888² | 6750 | 3956 | 58.61 |
| | | mussel | Mytilus edulis | | | 1998 | 29.60 |
| M18 | Mytilus chilensis | Blue mussel | Mytilus galloprovincialis | 53710² | 51670 | 50733 | 98.19 |
| M19 | not declared | Blue mussel | Mytilus galloprovincialis | 57238² | 54822 | 53829 | 98.19 |
| M20 | Mytilus spp. | Blue mussel | Mytilus galloprovincialis | 72113² | 69576 | 68969 | 99.13 |
| M21 | Mytilus edulis | Mussel | Mytilus galloprovincialis | 51328² | 49908 | 49459 | 99.10 |
| M22 | Mytilus galloprovincialis | Blue mussel | Mytilus galloprovincialis | 115950² | 110777 | 109262 | 98.63 |
| | | | Mytilus edulis | | | 1466 | 1.32 |
| M24 | Mytilus chilensis | Blue mussel in | Mytilus galloprovincialis | 113942² | 107150 | 94449 | 88.15 |
| | | tomato sauce | Mytilus edulis | | | 12505 | 11.67 |
| M28 | not declared | Dry cat food | Pecten spp. | 128693³ | 126380 | 79764 | 63.11 |
| | | with green | Mytilus galloprovincialis | | | 40450 | 32.01 |
| | | lipped mussel | Perna canaliculus | | | 4712 | 3.73 |
| M35 | Mytilus chilensis | Mussel in | Mytilus galloprovincialis | 197899³ | 190771 | 189540 | 99.35 |
| | | tomato sauce | | | | | |
| M39 | Mytilus chilensis | Blue mussel | Mytilus galloprovincialis | 182612³ | 175982 | 96502 | 54.84 |
| | | | Mytilus edulis | | | 75204 | 42.73 |
| M40 | Mytilus edulis | Blue mussel | Mytilus galloprovincialis | 182958³ | 179399 | 178024 | 99.23 |
| S41 | Placopecten magellanicus | Deep-sea scallop | Placopecten magellanicus | 143794² | 132140 | 131583 | 99.58 |
| S43 | Pecten maximus | Great scallop | Mizuhopecten yessoensis | 122156² | 113706 | 113128 | 99.49 |
| S44 | Pecten spp. | Great scallop | Mizuhopecten yessoensis | 2873135² | 2718126 | 2717426 | 99.97 |
| S45 | Placopecten magellanicus | Deep-sea scallop | Placopecten magellanicus | 111673² | 107119 | 106632 | 99.55 |
| S48 | Patinopecten yessoensis | Great scallop/ | Mizuhopecten yessoensis | 47397² | 41076 | 407873 | 99.51 |
| | | Yesso scallop | | | | | |
| S51 | not declared | Great scallop | Placopecten magellanicus | 51565² | 45007 | 44915 | 99.80 |
| S52 | Patinopecten yessoensis | Great scallop | Mizuhopecten yessoensis | 46673² | 39769 | 39627 | 99.64 |
| S53 | Pecten spp. | Great scallop | Mizuhopecten yessoensis | 42857² | 36443 | 35265 | 96.77 |
| S54 | Placopecten magellanicus | Great scallop | Placopecten magellanicus | 55475² | 48703 | 47915 | 98.38 |
| S56 | not declared | Great scallop | Placopecten magellanicus | 1268169³ | 1061137 | 1060653 | 99.95 |
| S57 | Placopecten magellanicus | Great scallop | Pecten spp. | 174497³ | 171299 | 170404 | 99.48 |
| S60 | not declared | Deep-sea scallop | Placopecten magellanicus | 364474³ | 350953 | 350869 | 99.98 |
| S61 | Patinopecten yessoensis | Great scallop | Mizuhopecten yessoensis | 159145³ | 152930 | 152849 | 99.95 |

¹value of two replicates;
²samples were analysed with the MiSeq instrument;
³samples were analysed with the iSeq instrument

### Example 2—Crustacean

In example 2, the identification of Crustacean in food samples according to the invention is shown. The identification was done similar to example 1 using the primer set specific for the identification of seafood species of the respective family in a singleplex setup (two forward primer SEQ ID NO: 1 and SEQ ID NO: 2; and one reverse primer SEQ ID NO: 15). The final concentration of two forward Crustacean primers (SEQ ID NO: 1 and SEQ ID NO: 2) was 0.2 μM each and 0.4 μM for reverse Crustacean primer (SEQ ID NO: 15). Then, the primers of Crustacean und Cephalopods (two Crustacean forward primers (SEQ ID NO: 1 and SEQ ID NO: 2), one Crustacean reverse primer (SEQ ID NO: 15), one Cephalopod forward primer (SEQ ID NO: 3 respectively SEQ ID NO: 4 and SEQ ID NO: 5) and one Cephalopod reverse primer (SEQ ID NO: 16)) were combined in a duplex assay. Furthermore, duplex setups are shown for the combined identification of species of the family of Crustacean and Gastropoda (two forward Crustacean primer SEQ ID NO: 1 and SEQ ID NO: 2, one reverse Crustacean primer SEQ ID NO: 15, one Gastropoda primer SEQ ID NO: 6 and two reverse Gastropoda primer SEQ ID NO: 17 and SEQ ID NO: 18) and also for the combined identification of species of the family of Crustacean and Cephalopods (two Crustacean forward primers (SEQ ID NO: 1 and SEQ ID NO: 2), one Crustacean reverse primer (SEQ ID NO: 15), one Cephalopod forward primer (SEQ ID NO: 3 respectively SEQ ID NO: 4 and SEQ ID NO: 5) and one Cephalopod reverse primer (SEQ ID NO: 16)). Sanger Sequencing was performed as a control experiment for the identification of seafood species.

### Results

The results are shown in the following Tables 10 and 11.

TABLE 10

| | | | | | | | Duplex | Duplex |
|---|---|---|---|---|---|---|---|---|
| | | | | | | Singelplex | Crustacean + | Crustacean + |
| | Product Description | | | | | Crustacean | Gastropoda | Cephalopods |
| | | Product | | Sanger Sequencing | | [MiSeq] | [MiSeq] | [MiSeq] |
| Sample | Scientific name | description | | Customer | Sanger 16S | Customer | Customer | Customer |
| No | of species | [Eng] | Treatment | Database | [310 bp] | Database | Database | Database |
| 3 | Nephrops | Norway lobster/ | cooled | Nephros | no result | Nephros | Nephros | Nephros |
| | norvegicus | scampie | | norvegicus | | norvegicus | norvegicus | norvegicus |
| 14 | Procambarus | Lusiana crayfish | cooled | Procambarus | Procambarus | Procambarus | not determined | Procambarus |
| | clarkii | | | clarkii | clarkii | clarkii | | clarkii |
| 15 | Panulirus argus | Crayfish | cooled | Panulirus argus | no result | Panuliurs spp | Panuliurs argus | Panuliurs argus |
| 17 | Homarus | Lobster | frozen | Homarus | Homarus | Homarus | Homarus | Homarus |
| | americanus | | | americanus | americanus | americanus | americanus | americanus |
| 20 | | Swimming crab | processed | Monomia | no result | Monomia | Monomia | |
| | | | | gladiator | | gladiator | gladiator | |
| 26 | Paralithodes | Kamchatka Crab/ | cooled | Paratithodes | no result | Paratithodes | Paratithodes | Paratithodes |
| | camtschaticus | King Crab | | camtschaticus | | camtschaticus | camtschaticus | camtschaticus |
| 40 | Chionoecetes | Snow crab | frozen | Chionnoecetes | no result | Chionnoecetes | Chionnoecetes | Chionnoecetes |
| | opilio | | | opilio | | spp | spp | spp |
| | | | | Chionocetes | | | | |
| | | | | bairdi | | | | |
| 45 | Procambarus | Crayfish | frozen | no result | (Pont)Astacus | Pontastacus | Pontastacus | Pontastacus |
| | clarkii | | | | leptodactylus | leptodactylus | leptodactylus | leptodactylus |
| 66 | Homarus | European lobster | frozen | Homarus | no result | Homarus | Homarus | Homarus |
| | gammarus | | | gammarus | | gammarus | gammarus | gammarus |

TABLE 11

| | Product Description | | | | |
|---|---|---|---|---|---|
| | Scientific | Product | | Sanger Sequencing | |
| Sample | name | description | Treat- | Customer | Sanger 16S |
| No | of species | [Eng] | ment | Database | [310 bp] |
| 4 | Pleoticus | Argentine red | frozen | Pleoticus | Pleoticus |
| | muelleri | shrimp | | muellerie | muellerie |
| 6 | Penaeus | Partei | cooled | Penaeus | Penaeus |
| | monodon | Crevetten | | monodon | monodon |
| 8 | Macrobrachium | Rosenberg | cooled | Macrobrachium | Macrobrachium |
| | rosenbergii | freshwater | | rosenbergii | rossenbergeri |
| | | shrimp | | | |
| 12 | Pandalus | Ice sea | cooled | Panadalus | Panadalus |
| | borealis | shrimps | | borealis | borealis = |
| | | | | | Panadaluseous |
| 16 | Crangon | North Sea | cooled | Crangon | no result |
| | crangon | crab | | crangon | |
| 31 | Litopenaeus | White Tiger | frozen | Penaeus | Litopenaeus |
| | vannamei | Shrimp | | vannamei | vanamei |
| 33 | Macrobrachium | Rosenberg | cooled | Peanaeus | Peanaeus |
| | rossenbergerii | freshwater | | monodon | monodon |
| | | shrimp | | | |
| 34 | Litopenaeus | White Tiger | cooled | Penaeus | Penaeus |
| | vannamei | Shrimp | | vannamei | vannamei |
| 36 | Metapenaeus | Shrimp | cooled | Mierspenaeopsis | Parapenaeopsis |
| | monoceros | | | hardwickii | sculptilis |
| | | | | Xiphopenaeus | Parapenaeopsis |
| | | | | kroyeri | hardwickii |
| 38 | Heterocarpus | Shrimp | cooled | Heterocarpus | no result |
| | reedi | | | spp. | |
| 42 | Penaeus | Tiger shrimp | frozen | Penaeus | Peanaeus |
| | monodon | | | vannamei | monodon |
| 43 | Macrobrachium | Rosenberg | frozen | Peanaeus | Peanaeus |
| | rossenbergerii | freshwater | | monodon | monodon |
| | | shrimp | | | |
| 50 | Penaeus | Organic Black | frozen | Panaeus | Panaeus |
| | monodon | Tiger Prawn | | monodon | monodon |
| 58 | Crangon | North Sea crab | cooled | Crangon | no result |
| | crangon | | | crangon | |
| 59 | Crangon | North Sea crab | cooled | Crangon | no result |
| | crangon | | | crangon | |
| 64 | Aristaeopsis | Carabineros | cooled | Aristaeopsis | Aristaeopsis |
| | edwardsiana | | | edwardsiana | edwardsiana |
| | | | | | Aristaeomorpha |
| | | | | | foliacea |
| 65 | Pleoticus | Shrimp ring | frozen | Pleoticus | no result |
| | muelleri | | | muelleri | |
| 70 | Penaeus | Pacific white | cooled | Pleoticus | no result |
| | occidentalis | shrimp | | muelleri | |
| 71 | Penaeus | white shrimp | cooled | Penaeus | Penaeus |
| | notialis | | | duorarum | duorarum |
| | | | | | Penaeus nr. |
| | | | | | notialis |
| 73 | Penaeus | Greenland | cooled | Pandalus | Pandalus eous = |
| | borealis | shrimp | | borealis | Pandalus borealis |
| 76 | Dendrobranchiata | Red shrimp | processed | | not determined |
| | (Suborder) | | | | |

| | | Duplex | Duplex |
|---|---|---|---|
| | Singelplex | Crustacea + | Crustacean + |
| | Crustacean | Gastropoda | Cephalopods |
| | [MiSeq] | [MiSeq] | [MiSeq] |
| Sample | Customer | Customer | Customer |
| No | Database | Database | Database |
| 4 | Pleoticus | not determined | Pleoticus |
| | muellerie | | muellerie |
| 6 | no result | Penaeus | Penaeus |
| | | monodon | monodon |
| 8 | Macrobrachium | not determined | Macrobrachium |
| | rosenbergii | | rosenbergii |
| 12 | Pandalus spp. | not determined | Pandalus spp. |
| 16 | Crangon crangon | Crangon crangon | Crangon |
| | | | crangon |
| 31 | Penaeus | Litopenaeus | Litopenaeus |
| | vannamei | vannamei | vannamei |
| 33 | Penaeus spp. | Genus Penaeus | Penaeus spp. |
| | no match to | no match to | no match to |
| | Rosenbergerii | Rosenbergerii | Rosenbergerii |
| 34 | Penaeus | Penaeus | Penaeus |
| | vannamei | vannamei | vannamei |
| 36 | Ganjampenaeopsis | Ganjampenaeopsis | Ganjampenaeopsis |
| | uncta | uncta | uncta |
| 38 | Heterocarpus | Heterocarpus | Heterocarpus |
| | spp. | spp. | spp. |
| 42 | Penaeus | Penaeus | Penaeus |
| | vannamei | vannamei | vannamei |
| 43 | Genus Penaeus | Genus Penaeus | Genus Penaeus |
| | no match to | no match to | no match to |
| | Rosenbergerii | Rosenbergerii | Rosenbergerii |
| 50 | Penaeus spp. | not determined | Penaeus spp. |
| 58 | Crangon crangon | Crangon crangon | Crangon crangon |
| 59 | Crangon crangon | Crangon crangon | Crangon crangon |
| 64 | Aristaeopsis | Aristaeopsis | Aristaeopsis |
| | spp | spp | spp) |
| 65 | no result | Pleoticus | not determined |
| | | muellerie | |
| 70 | Pleoticus muelleri | Pleoticus | Pleoticus |
| | | muellerie | muellerie |
| 71 | no result | Penaeus spp. | Penaeus spp. |
| 73 | no result | Pandalus spp. | Pandalus spp. |
| 76 | Penaeus | Penaeus | Penaeus |
| | vannamei | vannamei | vannamei |

### Example 3—Cephalopods

In example 3, the identification of Cephalopods in food samples according to the invention is shown. The identification was done similar to example 1 using the primer set specific for the identification of Cephalopods seafood species of the respective family in a singleplex setup (one forward primer SEQ ID NO: 3 respectively SEQ ID NO: 4 and SEQ ID NO: 5; and one reverse primer SEQ ID NO: 16). The final concentration of forward (SEQ ID NO: 3 respectively SEQ ID NO: 4 and SEQ ID NO: 5) and reverse primer (SEQ ID NO: 16) was 0.2 μM. Furthermore, a duplex setup is shown for the combined identification of species of the family of Crustacean and Cephalopods. The primers of Crustacean und Cephalopods (two Crustacean forward primers (SEQ ID NO: 1 and SEQ ID NO: 2), one Crustacea reverse primer (SEQ ID NO: 15), one Cephalopod forward primer (SEQ ID NO: 3 respectively SEQ ID NO: 4 and SEQ ID NO: 5) and one Cephalopod reverse primer (SEQ ID NO: 16)) were combined in a duplex assay. Sanger Sequencing was performed as a control experiment for the identification of seafood species.

### Results

The results are shown in the following Table 12.

TABLE 12
Cephalopods

| | Product Description | | | | |
|---|---|---|---|---|---|
| Sam- | | Product | | Sanger Sequencing | |
| ple | Scientific name | description | Treat- | Customer | Sanger COI |
| No | of species | [Eng] | ment | Database | [720 bp] |
| 2 | Loligo edulis | Squid cleaned | frozen | Illex illecebrosus | Uroteuthis |
| | | | | Uroteuthis | chinensis |
| | | | | (photololigo) | |
| | | | | chinensis | |
| 7 | Octopus vulgaris | Octopus tentacle | cooled | no result | Octopus vulgaris |
| 9 | Loligo chinensis | Squid | cooled | Doryteuthis | kein Ergebnis |
| | | | | (Amerigo) gahi | |
| 24 | Loligo duvauceli | Squid | frozen | Loligo duvauceli | Loligo gahi |
| 28 | Sepiella japonica | Sepie | frozen | Sepiella inermis | Sepiella inermis |
| 29 | Octopus vulgaris | Octopus | cooled | Octopus vulgaris | Octopus vulgaris |
| 32 | Sepia officinalis | Squid | cooled | Sepia pharaonis | Sepia pharaonis |
| 35 | Loligo vulgaris | Squid | cooled | Doryteuthis | Loligo gahi |
| | | | | (Amerigo) gahi | |
| 37 | Octopus aegina | Octopus | cooled | Amphioctopus | Octopus aegina |
| | | | | aegina | |
| 41 | Loligo opalescens | Squid | frozen | Doryteuthis | kein Ergebnis |
| | | | | opalescens | |
| 44 | Sepiella inermis | Sepie | frozen | Sepiella inermis | Sepiella inermis |
| 48 | Octopus maya | Ocotpus | frozen | Octopus maya | Octopus maya |
| 51 | Sepiella inermis | Sepie | frozen | Sepiella inermis | Sepiella inermis |
| 56 | Loligo edulis | Squid | frozen | Illex illecebrosus | Loligo |
| | | | | Uroteuthis | chinensis |
| | | | | (photololigo) | |
| | | | | chinensis | |
| 60 | Eledone | Musky octopus in | processed | Amphioctopus | Amphioctopus |
| | moschata | oil | | aegina | aegina |
| 69 | Loligo chinensis | Squid | cooled | Illex illecebrosus | Loligo chinensis |
| | | | | Uroteuthis | |
| | | | | (photololigo) | |
| | | | | chinensis | |
| 72 | Loligo galhi | Squid | cooled | Doryteuthis | Loligo gali |
| | | | | (Amerigo) gahi | |
| 124 | | fried calamari | processed | not determined | not determined |
| 129 | | Ocopus | processed | not determined | not determined |
| | | Carpaccio | | | |
| 132 | Uroteuthis | Squid tubes | frozen | not determined | not determined |
| | duvaucelii | | | | |
| 137 | Dosidicus gigas | Squid in ink | processed | not determined | not determined |
| | | sauce | | | |
| 141 | Dosidicus gigas | Squid in | processed | not determined | not determined |
| | | vegetable oil | | | |
| 142 | Dosidicus gigas | Squid in its own | processed | not determined | not determined |
| | | Ink | | | |
| 149 | | Squid in ink sauce | processed | not determined | not determined |
| 156 | Octopus | Musky octopus in | processed | not determined | not determined |
| | membranaceus | Oil | | | |
| 194 | | fried calamari | processed | not determined | not determined |
| | | Rings | | | |

| | | Duplex | Duplex |
|---|---|---|---|
| | Singelplex | Crustacean + | Crustacean + |
| | Cephalopods | Cephalopods | Cephalopods |
| Sam- | [MiSeq] | [MiSeq] | [Iseq] |
| ple | Customer | Customer | Customer |
| No | Database | Database | Database |
| 2 | no result | no result | Illex illecebrosus |
| | | | Uroteuthis |
| | | | (photololigo) |
| | | | chinensis |
| 7 | no result | Octopus spp | Octopus spp. |
| 9 | no result | Doryteuthis | Doryteuthis |
| | | (Amerigo) gahi | (Amerigo) gahi |
| 24 | Loligo spp. | Loligo spp. | not determined |
| 28 | no result | Sepiella inermis | Sepiella inermis |
| 29 | no result | Octopus spp. | Octopus spp. |
| 32 | Sepia spp. | Sepia spp | not determined |
| 35 | Doryteuthis | Doryteuthis | not determined |
| | (Amerigo) gahi | (Amerigo) gahi | |
| 37 | Amphioctopus | not determined | Amphioctopus |
| | aegina | | aegina |
| 41 | Doryteuthis | not determined | Doryteuthis |
| | opalescens | | opalescens |
| 44 | Sepiella inermis | not determined | Sepiella inermis |
| 48 | Octopus maya | not determined | Octopus maya |
| 51 | Sepiella inermis | not determined | Sepiella inermis |
| 56 | Uroteuthis | not determined | Uroteuthis |
| | chinensis | | chinensis |
| | llex ilecebrosus | | llex ilecebrosus |
| 60 | Amphioctopus | Amphioctopus | not determined |
| | aegina | aegina | |
| 69 | Uroteuthis | Uroteuthis | not determined |
| | chinensis | chinensis | |
| | llex ilecebrosus | llex ilecebrosus | |
| 72 | Doryteuthis | not determined | Doryteuthis |
| | Amerigo gahi | | (Amerigo) gahi |
| 124 | not determined | not determined | Doryteuthis |
| | | | (Amerigo) gahi |
| 129 | not determined | not determined | Octopus cyanea |
| 132 | not determined | not determined | Uroteuthis spp. |
| 137 | not determined | not determined | Dosidicus gigas |
| 141 | not determined | not determined | Dosidicus gigas |
| 142 | not determined | not determined | Todarodes |
| | | | pacificus |
| 149 | not determined | not determined | Todarodes |
| | | | pacificus |
| 156 | not determined | not determined | Amphioctopus |
| | | | spp. |
| 194 | not determined | not determined | Dosidicus gigas |

### Example 4—Gastropoda

In example 4, the identification of Gastropoda in food samples according to the invention is shown. The identification was done similar to example 1 using the primer set specific for the identification of seafood species of the respective family in a singleplex setup (one forward primer SEQ ID NO: 6 and two reverse primer SEQ ID NO: 17 and SEQ ID NO: 18). The final concentration of forward Gastropoda primer (SEQ ID NO: 6) was 0.4 μM and 0.2 μM each for the two reverse Gastropoda primer (SEQ ID NO: 17 and SEQ ID NO: 18). Furthermore, a duplex setup is shown for the combined identification of species of the family of Crustacean and Gastropoda. Sanger Sequencing was performed as a control experiment for the identification of seafood species.

### Results

The results are shown in the following Table 13.

TABLE 13
Gastropoda

| | | | | | | | Duplex | |
|---|---|---|---|---|---|---|---|---|
| | | | | | | Singelplex | Crustacean + | |
| | Product Description | | | | | Gastropoda | Gastropoda | Gastropoda |
| | Scientific | Product | | Sanger Sequencing | | [MiSeq] | [MiSeq] | [Iseq] |
| Sample | name | description | | Customer | Sanger COI | Customer | Customer | Customer |
| No | of species | [Eng] | Treatment | Database | [720 bp] | Database | Database | Database |
| 5 | Helix lucorum | Snail with butter | processed | Helix lucorum | no result | Helix spp. | Helix spp | not determined |
| | | and white wine | | | | | | |
| 39 | | Agate snail | cooled | Achatina reticulata | no result | Achantina spp | Achantina spp. | not determined |
| 53 | Helix lucorum | Roman snail | frozen | Helix promatia | Helix promatia | Helix spp. | no result | Helix spp. |
| 75 | | Food supplement | processed | not determined | not determined | no result | Helix spp. | Helix thessalica |
| | | with snail powder | | | | | | |
| 144 | | Roman snail | processed | not determined | not determined | not determined | not determined | Helix spp. |
| 145 | | Vine snail with | processed | not determined | not determined | not determined | not determined | Helix spp. |
| | | balsamic onion | | | | | | |
| 146 | | Agate snail | processed | not determined | not determined | not determined | not determined | Achantina fulica |

### Example 5—Veneridae

In example 5, the identification of Veneridae in food samples according to the invention is shown. The identification was done similar to example 1 using the primer set specific for the identification of seafood species of the respective family in a singleplex setup (five forward primer SEQ ID NO: 7, SEQ ID NO: 8, SEQ ID NO: 9, SEQ ID NO: 10, and SEQ ID NO: 11 and three reverse primer SEQ ID NO: 19, SEQ ID NO: 20, and SEQ ID NO: 21). The final concentration of forward primer (SEQ ID NO: 7, SEQ ID NO: 8, SEQ ID NO: 9, SEQ ID NO: 10, and SEQ ID NO: 11) was 0.2 μM each, 0.2 μM each for two reverse primer (SEQ ID NO: 20 and SEQ ID NO: 21) and 0.6 μM for the reverse primer (SEQ ID NO: 19). Sanger Sequencing was performed as a control experiment for the identification of seafood species.

### Results

The results are shown in the following Table 14.

TABLE 14
Veneridae

| | Product Description | | | | | Singelplex Veneridae |
|---|---|---|---|---|---|---|
| | | Product | | Sanger Sequencing | | [MiSeq] |
| Sample | Scientific name | description | | Customer | Sanger COI | Customer |
| No | of species | [Eng] | Treatment | Database | [720 bp] | Database |
| 10 | Ensis ensis | Clam | cooled | Ensis directus | Ensis directus | Ensis spp |
| | | | | Ensis terranovensis | | |
| 11 | Ruditapes philippinarum | Venus clam | cooled | Ruditapes philippinarum | no result | Ruditapes philipinarum |
| 18 | not declared | Pickled cockles | processed | Cerastoderma edule | no result | Cerastoderma edule |
| 21 | Meretrix lyrata | Venus clam | frozen | Meretrix lyrate | Meretrix lyrata | Meretrix lyrata |
| 52 | Callista Chione | Venus clam | frozen | no result | no result | no result |
| 61 | Solen marinatus | Vagina shell | cooled | Ensis directus | Ensis directus | Ensis spp |
| | | | | Ensis terranovensis | | |
| 63 | Ruditapes philippinarum | Venus clam | cooled | Ruditapes philippinarum | kein Ergbenis | Ruditapes philipinarum |
| 67 | Cerastoma edulis | Cockle | cooled | Cerastoma edulis | Cerastoma edulis | Cerastoderma edule |
| 68 | Chamelea gallina | Venus clam | cooled | Chamelea gallina | Chamelea gallina | Chamelea gallina |
| 86 | Cerastoderma edule | Cockle | cooled | not determined | not determined | Cerastoderma edule |
| 87 | Ruditapes philipinarum | Carpet shell | cooled | not determined | not determined | Ruditapes philipinarum |
| 93 | Ensis direktus | Seaside | cooled | not determined | not determined | Ensis spp |
| 95 | Venus spp | Venus clam | cooled | not determined | not determined | Ruditapes philipinarum |
| 96 | Metrix lyrate | Brown clam | frozen | not determined | not determined | Metrix lyrate |
| 100 | not declared | Clams au naturel | processed | not determined | not determined | Chamelea gallina |
| 121 | not declared | Clams au naturel | cooled | not determined | not determined | Ruditapes philipinarum |

### REFERENCES

Dobrovolny, S.; et al. Development of a DNA metabarcoding method for the identification of fifteen mammalian and six poultry species in food. Food Chemistry 2019, 272, 354-361, doi:10.1016/j.foodchem.2018.08.032.
Edgar, R. C. Search and clustering orders of magnitude faster than BLAST. Bioinformatics 2010, 26, 2460-2461, doi:10.1093/bioinformatics/btq461.
Espiñeira, M.; et al., Development of a method for the genetic identification of commercial bivalve species based on mitochondrial 18S rRNA sequences. J. Agric. Food Chem. 2009, 57, 495-502, doi:10.1021/jf802787d.
Fernández, A.; et al. Identification of the clam species Ruditapes decussatus (Grooved carpet shell), Venerupis pullastra (Pullet carpet shell), and Ruditapes philippinarum (Japanese carpet shell) by PCR-RFLP. J. Agric. Food Chem. 2000, 48, 3336-3341, doi:10.1021/jf0002185.
Gense K. et al., Development of a DNA Metabarcoding Method for the Identification of Bivalve Species in Seafood Products, Foods, 2021, Vol. 10(11), p. 2618
Klapper R. et al., Verification of authenticity: A rapid identification method for commercial scallop species through multiplex real-time PCR, Food Control, 2021, Vol. 121
Marin A. et al., The variable 5′ end of the 16S rRNA gene as a novel barcoding tool for scallops (Bivalvia, Pectinidae), Fish Sci, 2015, Vol. 81(1), pp. 73-81
Marin A. et al., Novel decaplex PCR assay for simultaneous detection of scallop species with species-specific primers targeting highly variable 5′ end of the 16S rRNA gene, Aquaculture Research, 2017, Vol. 48(3), pp. 920-930
Martin, M. Cutadapt removes adapter sequences from high-throughput sequencing reads. EMBnet j. 2011, 17, 10, doi:10.14806/ej.17.1.200.
Näumann, G.; Stumme, B.; Rehbein, H. Differenzierung von Kammmuscheln durch DNA-Analyse. Informationen aus der Fischereiforschung—Information on Fishery Research 2012, 1-7, doi:10.3220/Infn59_1-7_2012.
Pardo, Miguel Ángel; Jiménez, Elisa; Pérez-Villarreal, Begoña: Misdescription incidents in seafood sector. In: Food Control, 2016, 62, S. 277-283. DOI: 10.1016/j.foodcont.2015.10.048.
Robson, K., Dean, M., Haughey, S., Elliott, C. A comprehensive review of food fraud terminologies and food fraud mitigation guides. In: Food Control, 2021, 120, S. 107516. DOI: 10.1016/j.foodcont.2020.107516.
Rodriguez, E. M.; Ortea, I. Food Authentication of Seafood Species. In Proteomics in food science: From farm to fork, 3rd; Colgrave, M. L., Ed.; Academic Press: London, 2017; pp 331-342, ISBN 9780128040072.
Spielmann G. et al., Comparison of three DNA marker regions for identification of food relevant crustaceans of the order Decapoda, European Food Research and Tech., 2019, Vol. 245(5), pp. 987-995
Stephan, R.; et al. Rapid and reliable species identification of scallops by MALDI-TOF mass spectrometry. Food Control 2014, 46, 6-9, doi:10.1016/j.foodcont.2014.04.047.
Sun S. et al., Molecular Identification of Dried Shellfish Products Sold on the Market Using DNA Barcoding, J. Ocean Univ. China, 2021, Vol. 20(4), pp. 931-938
Wen J. et al., An insight into the Chinese traditional seafood market: Species characterization of cephalopod products by DNA barcoding and phylogenetic analysis using COI and 16SrRNA genes, Food Control, 2017, Vol. 82, pp. 333-342
Fernandes, T. J. R.; Amaral, J. S.; Mafra, I. DNA barcode markers applied to seafood authentication: an updated review. Critical reviews in food science and nutrition 2020, 1-32. DOI: 10.1080/10408398.2020.1811200

1. A method for identifying seafood species of different origin and processing degree in a food sample, comprising the steps of:
a) isolating DNA from the sample,
b) amplifying fragments of said DNA with at least 2 primer sets (ps) selected from the group of
i. ps 1 for identifying seafood species of the family of Crustaceans comprising one or more primer pairs of one forward primer selected from any one of SEQ ID NOs: 1 and 2, and a corresponding reverse primer SEQ ID NO: 15 and/or reverse complement sequences of the primer sequences;
ii. ps 2 for identifying seafood species of the family of Cephalopods comprising one or more primer pairs of one forward primer selected from any one of SEQ ID NOs: 3 to 5, and a corresponding reverse primer SEQ ID NO: 16 and/or reverse complement sequences of the primer sequences;
iii. ps 3 for identifying seafood species of the family of Gastropoda comprising one or more primer pairs of forward primer SEQ ID NO: 6, and one reverse primer selected from any one of SEQ ID NOs: 17 to 18 and/or reverse complement sequences of the primer sequences;
iv. ps 4 for identifying seafood species of the family of Veneridae comprising one or more primer pairs of one forward primer selected from any one of SEQ ID NOs: 7 to 11, and one reverse primer selected from any one of SEQ ID NOs: 19 to 21 and/or reverse complement sequences of the primer sequences;
v. ps 5 for identifying seafood species of the family of Ostreidae comprising the forward primer SEQ ID NO: 12 and a corresponding reverse primer SEQ ID NO: 22 and/or reverse complement sequences of the primer sequences;
vi. ps 6 for identifying seafood species of the family of Pectinidae comprising the forward primer SEQ ID NO: 13 and a corresponding reverse primer SEQ ID NO: 23 and/or reverse complement sequences of the primer sequences; and
vii. ps 7 for identifying seafood species of the family of Mytilidae comprising one or more primer pairs of forward primer SEQ ID NO: 14, and one reverse primer selected from any one of SEQ ID NOs: 24 to 25 and/or reverse complement sequences of the primer sequences,
c) sequencing the amplified DNA fragments of step b), and
d) identifying the seafood species by comparison of the sequences obtained by steps a) to c) with reference sequences of seafood species.

2. The method of claim 1, wherein amplifying of fragments of DNA in step b) is performed by a polymerase chain reaction (PCR) comprising 25-30 cycles at an annealing temperature of 60-65° C.

3. The method of claim 1, wherein amplifying fragments of the DNA in step b) is performed with 3, 4, 5, 6, or 7 primer sets.

4. The method of claim 1, wherein the amplified DNA fragments of step b) are 16S rDNA fragments.

5. The method of claim 1, wherein the reference sequences of seafood species comprise the DNA sequence of the 16S rDNA of said seafood species.

6. The method of claim 5, wherein the reference sequences are any one of SEQ ID NOs: 28 to 1153, or any combination thereof.

7. The method of claim 1, wherein the identified species of the family of Crustaceans are selected from Varuna litterata, Hemisquilla ensigera, Gonodactylus smithii, Pullosquilla thomassini, Chorisquilla trigibbosa, Telmessus acutidens, Lithodes aequispinus, Panulirus echinatus, Jasus paulensis, Jasus caveorum, Parastacus pilimanus, Parastacus brasiliensis, Parastacus defossus, Parastacus nicoleti, Gonodactylus graphurus, Jasus lalandii, Lopholithodes mandtii, Lithodes spp., Lithodes maja, Jasus edwardsii, Panulirus regius, Panulirus pascuensis, Panulirus laevicauda, Panulirus gracilis, Panulirus guttatus, Panulirus femoristriga, Chionoecetes spp., Paralomis granulosa, Panulirus spp., Scyllarus arctus, Palinurus elephas, Episesarma mederi, Austropotamobius torrentium, Cycloachelous granulatus, Eriocheir recta, Cervimunida johni, Achelous fioridanus, Portunus sayi, Portunus anceps, Palinurus mauritanicus, Palinurus charlestoni, Pseudosquilla ciliata, Pleuroncodes monodon, Portunus ventralis, Achelous spinicarpus, Callinectes toxotes, Callinectes danae, Callinectes ornatus, Callinectes marginatus, Callinectes affinis, Callinectes rathbunae, Callinectes bocourti, Callinectes similis, Callinectes bellicosus, Callinectes arcuatus, Metanephrops armatus, Metanephrops mozambicus, Metanephrops japonicus, Metanephrops spp., Metanephrops binghami, Parastacus pugnax, Paranephrops zealandicus, Callinectes exasperatus, Palinurus spp., Sagmariasus verreauxi, Metanephrops rubellus, Metanephrops challengeri, Metanephrops neptunus, Metanephrops australiensis, Metanephrops arafurensis, Metanephrops boschmai, Metanephrops formosanus, Metanephrops sinensis, Lithodes ferox, Oratosquillina interrupta, Odontodactylus japonicus, Miyakella nepa, Erugosquilla woodmasoni, Clorida decorata, Dictyosquilla foveolata, Anchisquilla fasciata, Scyllarides herklotsii, Astacus astacus, Portunus hastatus, Achelous ordwayi, Carcinus maenas, Portunus inaequalis, Astacoides madagascariensis, Erimacrus isenbecki, Hemisquilla australiensis, Austrosquilla tsangi, Fallosquillafallax, Echinosquilla guerinii, Coronis scolopendra, Chorisquilla tweediei, Chorisquilla hystrix, Chorisquilla excavate, Busquilla plantei, Alima pacifica, Alima orientalis, Alachosquilla vicina, Gonodactylellus espinosus, Gonodactylellus affinis, Kempella mikado, Hemisquilla cahforniensis, Haptosquilla trispinosa, Haptosquilla glyptocercus, Gonodactylus platysoma, Gonodactylaceus falcatus, Gonodactylus childi, Gonodactylellus annularis, Odontodactylus scyllarus, Odontodactylus latirostris, Odontodactylus havanensis, Odontodactylus cultrifer, Neogonodactylus oerstedii, Neogonodactylus bredini, Neogonodactylus bahiahondensis, Lysiosquillina sulcata, Squilla rugosa, Raoulserenea spp., Raoulserenea oxyrhyncha, Pseudosquillopsis marmorata, Raoulserenea komaii, Protosquilla folini, Ibacus alticrenatus, Scyllarides nodifer, Scyllarides haanii, Scyllarides brasiliensis, Taku spinosocarinatus, Jasus frontalis, Procambarus paeninsulanus, Puerulus sewelli, Panulirus polyphagus, Panulirus longipes, Panulirus interruptus, Panulirus marginatus, Ibacus peronii, Ibacus chacei, Faxonella clypeata, Fallicambarus kountzeae, Arenaeus mexicanus, Cambarus tartarus, Chionoecetes tanneri, (Currently amended) Thenus unimaculatus, (Currently amended) Thenus indicus, Haptosquilla hamifera, Lithodes turritus, Bouchardina robisoni, Troglocambarus maclanei, Hobbseus yalobushensis, Hobbseus prominens, Charybdis spp., Hobbseus petilus, Faxonella creaseri, Thranita danae, Monomia petrea, Neogonodactylus wennerae, Xiphonectes pseudohastatoides, Gonodactylellus viridis, Gonodactylaceus ternatensis, Belosquilla laevis, Procambarus okaloosae, Procambarus morrisi, Procambarus milleri, Procambarus mancus, Procambarus lunzi, Hobbseus cristatus, Procambarus acutissimus, Faxonius pagei, Manningia pilaensis, Pontastacus leptodactylus, Procambarus zonangulus, Procambarus youngi, Procambarus seminolae, Procambarus pycnogonopodus, Procambarus orcinus, Procambarus pallidus, Alima maxima, Scyllarides deceptor, Monomia argentata, Xiphonectes pulchricristatus, Paralithodes platypus, Lopholithodes foraminatus, Faughniaformosae, Faughnia profunda, Bathysquilla crassispinosa, Eriocheir sinensis, Harpiosquilla harpax, Callinectes sapidus, Squilla mantis, Portunus trituberculatus, Panulirus japonicus, Cancer pagurus, Chionoecetes japonicus, Scylla tranquebarica, Scylla serrata, Eriocheir japonica, Eriocheir hepuensis, Cherax destructor, Squilla empusa, Lysiosquillina maculata, Gonodactylus chiragra, Panulirus homarus, Homarus americanus, Panulirus ornatus, Oratosquilla oratoria, Panulirus stimpsoni, Charybdis japonica, Scylla paramamosain, Scylla olivacea, Cherax quadricarinatus, Cherax cainii, Paralithodes brevipes, Paralithodes camtschaticus, Scyllarides latus, Procambarus clarkii, Procambarus fallax, Homarus gammarus, (Currently amended) Thenus orientalis, Lithodes nintokuae, Cherax cairnsensis, Cherax dispar, Cherax quinquecarinatus, Cherax robustus, Cherax monticola, Cherax glaber, Cherax holthuisi, Astacopsis gouldi, Portunus pelagicus, Paranephrops planifrons, Nephrops norvegicus, Ibacus ciliatus, Charybdis feriata, Metanephrops sibogae, Panulirus cygnus, Metanephrops thomsoni, Faxonius limosus, Squilloides leptosquilla, Cherax bicarinatus, Austropotamobius pallipes, Cherax tenuimanus, Cherax boesemani, Charybdis (Charybdis) natator, Procambarus acutus, Pacifastacus leniusculus, Munida gregaria, Panulirus versicolor, Faxonius rusticus, Portunus sanguinolentus, Procambarus alleni, Metacarcinus magister, Puerulus angulatus, Lupocycloporus gracilimanus, Monomia gladiator, Varuna yui, Panulirus argus, Munida isos, Scyllarides squammosus, Cambaroides similis, Charybdis bimaculata, Cambarus robustus, Thalamita sima, Thranita crenata, Orconectes luteus, Orconectes punctimanus, Orconectes sanbornii, Cherax spp., Cherax crassimanus, Cherax preissii, Munida spinosa, Munida asprosoma, Munida leagora, Munida alonsoi, Munida taenia, Munida gordoae, Munida zebra, Munida distiza, Munida psamathe, Munida thoe, Munida guttata, Munida stia, Munida ommata, Munida roshanei, Munida compressa, Munida clinata, Munida chydaea, Munida compacta, Munida eclepsis, Munida tyche, Munida philippinensis, Munida armilla, Munida mesembria, Munida spilota, Munida benguela, Munida endeavourae, Munida agave, Munida idyia, Munida militaris, Munida flinti, Munida congesta, Munida rubridigitalis, Munida iris, Munida microphthalma, Munida rufiantennulata, Munida pusilla, Munida remota, Munida leptosyne, Munida rosula, Munida munin, Munida valida, Munida proto, Enriquea leviantennata, Munida multilineata, Munida pagesi, Munida stomifera, Munida quadrispina, Munida tiresias, Munida psylla, Munida heteracantha, Paralomis formosa, Paralomis spinosissima, Paralomis birsteini, Paralomis hirtella, Scyllarus subarctus, Scyllarus pygmaeus, Scyllarus chacei, Scyllarus caparti, Scyllarus americanus, Episesarma palawanense, Episesarma singaporense, Austropotamobius fulcisianus orientalis, Achelous tumidulus, Achelous asper, Achelous sebae, Portunus acuminatus, Achelous tuberculatus, Achelous iridescens, Portunus xantusii, Achelous depressifrons, Achelous rufiremus, Achelous gibbesii, Portunus minimus, Achelous stanfordi, Achelous brevimanus, Portunus affinis, Achelous angustus, Achelous binoculus, Oratosquillina inornata, Oratosquillina asiatica, Oratosquillina anomala, Oratosquillina perpensa, Erugosquilla graham, Busquilla quadraticauda, Kempella stridulans, Gonodactylaceus graphurus, Gonodactylaceus randalli, Carcinus aestuarii, Menippe rumphii, Menippe nodifrons, Menippe spp., Procambarus liberorum, Procambarus toltecae, Procambarus curdi, Procambarus digueti, Procambarus nigrocinctus, Procambarus versutus, Procambarus gibbus, Cambarus pecki, Procambarus geminus, Charybdis acuta, Creaserinus fodiens, Fallicambarus jeanae, Creaserinus gordoni, Creaserinus caesius, Fallicambarus dissitus, Creaserinus danielae, Fallicambarus oryktes, Fallicambarus byersi, Creaserinus burrisi, Creaserinus gilpini, Fallicambarus harpi, Fallicambarus macneesei, Fallicambarus petilicarpus, Fallicambarus wallsi, Fallicambarus strawni, Fallicambarus devastator, Fallicambarus houstonensis, Fallicambarus hortoni, Arenaeus cribrarius, Cambarus spp., Cambarus deweesae, Cambarus striatus, Cambarus graysoni, Cambarus monongalensis, Cambarus pyronotus, Cambarus maculatus, Cambarus latimanus, Cambarus strigosus, Cambarus parrishi, Cambarus bouchardi, Cambarus fasciatus, Cambarus harti, Cambarus nerterius, Cambarus setosus, Cambarus batchi, Cambarus halli, Cambarus unestami, Cambarus reburrus, Cambarus gentry, Cambarus hubbsi, Cambarus friaufi, Cambarus obeyensis, Cambarus cracens, Cambarus asperimanus, Cambarus hobbsorum, Cambarus williami, Cambarus howardi, Cambarus obstipus, Cambarus girardianus, Cambarus cryptodytes, Cambarus sciotensis, Cambarus georgiae, Cambarus pristinus, Cambarus aculabrum, Cambarus englishi, Cambarus brachydactylus, Cambarus cumberlandensis, Cambarus dubius, Cambarus reflexus, Cambarus scotti, Cambarus longirostris, Cambarus hubrichti, Monomia lucida, Faughnia serenei, Harpiosquilla melanoura, Harpiosquilla annandalei, Cherax cuspidatus, Cherax paniaicus, Cherax lorentzi, Cherax albertisii, Cherax rotundus, Cherax leckii, Cherax murido, Cherax wasselli, Cherax parvus, Cherax pallidus, Cherax cartalacoolah, Cherax rhynchotus, Cherax pulcher, Cherax peknyi, Cherax setosus, Cherax misolicus, Cherax warsamsonicus, Cherax snowden, Cherax boschmai, Cherax nucifraga, Cherax barrette, Oratosquillafabricii, Astacopsis tricornis, Thalamita admete, Faxonius virilis, Thranita prymna, Astacopsis franklinii, Cambaroides schrenckii, Orconectes australis, Thalamita chaptalii, Zygita longifrons, Thalamita picta, Thalamita seurati, Thranita pelsarti, Orconectes barri, Faxonius ronaldi, Faxonius neglectus, Orconectes compressus, Orconectes forceps, Orconectes pellucidus, Neoeriocheir leptognathus, Penaeus kerathurus, Penaeus marginatus, Penaeus longistylus, Penaeus plebejus, Metapenaeopsis liui, Metapenaeopsis lamellata, Metapenaeopsis acclivis, Metapenaeopsis commensalis, Atypopenaeus stenodactylus, Aristeus antillensis, Solenocera vioscai, Penaeus chinensis, Penaeus spp., Metapenaeopsis barbata, Penaeus esculentus, Heteropenaeus longimanus, Atypopenaeus dearmatus, Funchalia taaningi, Xiphopenaeus kroyeri, Trachypenaeopsis mobilispinis, Rimapenaeus similis, Parapenaeus politus, Solenocera membranacea, Alcockpenaeopsis hungerfordii, Batepenaeopsis tenella, Pandalus platyceros, Metapenaeus moyebi, Metapenaeus joyneri, Pandalus montagui, Penaeus brasiliensis, Aristeus antennatus, Heterocarpus laevigatus, Heterocarpus lepidus, Funchalia villosa, Hemipenaeus carpenter, Mesopenaeus tropicalis, Pelagopenaeus balboae, Penaeus hathor, Metapenaeopsis provocatoria, Aristeus virilis, Aristeus alcock, Penaeus aztecus, Heterocarpus abulbus, Penaeus setiferus, Cerataspis monstrosus, Pleoticus robustus, Aristaeopsis edwardsiana, Solenocera necopina, Parapenaeus cayrei, Parapenaeus fissurus, Parapenaeus investigatoris, Parapenaeus fissuroides, Parapenaeus americanus, Heterocarpus ensifer, Kishinouyepenaeopsis cornuta, Parapenaeus perezfarfantae, Parapenaeus murrayi, Parapenaeus longipes, Parapenaeus spp., Heterocarpus chani, Heterocarpus sibogae, Heterocarpus dorsalis, Metapenaeopsis andamanensis, Metapenaeopsis coniger, Macrobrachium idella, Trachysalambria brevisuturae, Trachysalambria aspera, Trachysalambria albicoma, Euphausia superba, Solenocera hextii, Hymenopenaeus equalis, Rimapenaeus constrictus, Crangon crangon, Trachypenaeus anchoralis, Megokris spp., Trachysalambria longipes, Trachysalambria starobogatovi, Trachysalambria nansei, Trachysalambria malaiana, Trachysalambria spp., Trachysalambria parvispina, Crangon uritai, Pandalus borealis, Metapenaeus monoceros, Pandalus nipponensis, Hadropenaeus lucasii, Ganjampenaeopsis uncta, Solenocera annectens, Solenocera melantho, Parapenaeopsis stylifera, Penaeus japonicus, Penaeus brevirostris, Penaeus notialis, Penaeus duorarum, Penaeus schmitti, Artemesia longinaris, Penaeus subtilis, Penaeus stylirostris, Penaeus vannamei, Macrobrachium rosenbergii, Penaeus monodon, Pandalus hypsinotus, Heterocarpus spp., Pandalus jordani, Macrobrachium bullatum, Penaeus merguiensis, Metapenaeus ensis, Acetes chinensis, Macrobrachium nipponense, Penaeus cahforniensis, Macrobrachium lanchesteri, Pleoticus muelleri, Metapenaeus affinis, Hymenopenaeus neptunus, Penaeus indicus, Aristaeomorphafoliacea, Solenocera spp., Mierspenaeopsis hardwickii, Penaeus latisulcatus, Penaeus semisulcatus, Penaeus isabelae, Sicyonia lancifer, Metapenaeopsis dalei, Metapenaeopsis gerardoi, Parapenaeus longirostris, Pandalus eous, Pandalus miyakei, Pandalus japonicas, Pandalus glabrus, Pandalus teraoi, Pandalus ivanovi, Pandalus coccinatus, Pandalus formosanus, Pandalus chani, Pandalus spp., Pandalus longirostris, Pandalus latirostris, Metapenaeus spp., Palaemon spp., Palaemon serratus, Macrobrachium nipponense, Palaemon capensis, Palaemon sinensis, Palaemon annandalei, Palaemon gravieri, Palaemon serenus, Palaemon carinicauda, Palaemon pugio, Palaemon pandaiformis, Palaemon elegans, Palaemon longirostris, Palaemon peringueyi, Palaemon debilis, Palaemon carteri, Palaemon ritteri, Palaemon orientis, Macrobrachium gracilirostre, Palaemon vulgaris, Palaemon serrifer, Palaemon varians, Palaemon macrodactylus, Palaemon tonkinensis, Palaemon xiphias, Palaemon ivonicus, Palaemon pacificus, Palaemon atrinubes, Palaemon intermedius, Palaemon concinnus, Palaemon yuna, Palaemon antennarius, Palaemon dolospinus, Palaemon gracilis, Palaemon mundusnovus, Palaemon suttkusi, Palaemon zariquieyi, Macrobrachium australiense, Palaemon semmelinkii, Palaemon litoreus, Palaemon septemtrionalis, Palaemon guangdongensis, Palaemon hancocki, Palaemon vietnamicus, Palaemon texanus, Palaemon ortmanni, Palaemon turcorum, Palaemon kadiakensis, Macrobrachium asperulum, Macrobrachium australe, Macrobrachium olfersii, Macrobrachium jelskii, Macrobrachium villosimanus, Macrobrachium equidens, Macrobrachium potiuna, Macrobrachium malcolmsonii, Macrobrachium superbum, Macrobrachium striatum, Macrobrachium latidactylus, Macrobrachium hancocki, Macrobrachium acanthurus, Macrobrachium inflatum, Macrobrachium crenulatum, Macrobrachium carcinus, Macrobrachium americanum, Macrobrachium latimanus, Macrobrachium mammillodactylus, Macrobrachium faustinum, Macrobrachium heterochirus, Macrobrachium scabriculum, Macrobrachium digueti, Macrobrachium tenellum, Macrobrachium idae, Macrobrachium formosense, Macrobrachium dienbienphuense, Macrobrachium placidulum, Macrobrachium sintangense, Macrobrachium niphanae, Macrobrachium totonacum, Macrobrachium tuxtlaense, Macrobrachium vicconi, Macrobrachium villalobosi, Macrobrachium amazonicum, Macrobrachium canarae, Macrobrachium tratense, Macrobrachium forcipatum, Macrobrachium hirsutimanus, Macrobrachium borellii, Macrobrachium brasiliense, Macrobrachium aemulum, Macrobrachium handschini, Macrobrachium horstii, Macrobrachium ferreirai, Macrobrachium lanatum, Macrobrachium novaehollandiae, Macrobrachium tolmerum, Macrobrachium iheringi, Macrobrachium saigonense, Macrobrachium nattereri, Macrobrachium aracamuni, Macrobrachium inpa, Macrobrachium depressimanum, Macrobrachium surinamicum, Macrobrachium denticulatum, Macrobrachium pilimanus, Macrobrachium ohione, Macrobrachium hainanense, Macrobrachium lepidactyloides, Macrobrachium jaroense, Macrobrachium esculentum, Macrobrachium maculatum, Macrobrachium edentatum, Macrobrachium grandimanus, Macrobrachium malayanum, Macrobrachium meridionale, Macrobrachium neglectum, Macrobrachium platycheles, Macrobrachium naso, Macrobrachium placidum, Macrobrachium yui, Macrobrachium shokitai, Macrobrachium sundaicum, Macrobrachium rude, Macrobrachium lamarrei, Macrobrachium sankolli, Macrobrachium gangeticum, Trachysalambria palaestinensis, Euphausia pacifica, Euphausia lucens, Euphausia vallentini, Euphausia triacantha, Euphausia longirostris, Euphausia similis, Euphausia recurve, Euphausia krohni, Euphausia frigida, Euphausia gibboides, Euphausia eximia, Euphausia americana, Euphausia tenera, Euphausia pseudogibba, Euphausia hemigibba, Euphausia brevis, Hymenopenaeus debilis and Nematopalaemon tenuipes.

8. The method of claim 1, wherein the identified species of the family of Cephalopods are selected from Loligoforbesii, Nototodarus sloanii, Sepia spp., Sepia lorigera, Sepia pardex, Rossia pacifica, Berryteuthis magister, Eledone massyae, Sepia robsoni, Loligo reynaudii, Doryteuthis (Amerigo) pealeii, Doryteuthis (Amerigo) gahi, Sepiola rondeletii, Adinaefiola ligulata, Sepia smithi, Sepia elliptica, Eledone palari, Eledone moschata, Rossia palpebrosa, Gonatus madokai, Gonatus kamtschaticus, Eledone cirrhosa, Sepia elegans, Rossia bipillata, Sepiola atlantica, Lolliguncula (Lolliguncula) panamensis, Octopus maya, Ilex illecebrosus, Nototodarus gouldi, Gonatopsis octopedatus, Illex coindetii, Berryteuthis anonychus, Gonatus fabricii, Lusepiola birostrata, Octopus tetricus, Uroteuthis (Photololigo) sibogae, Doryteuthis (Doryteuthis) pleii, Doryteuthis sanpaulensis, Doryteuthis (Amerigo) surinamensis, Octopus hubbsorum, Macrotritopus defilippi, Octopus insularis, Loliolus (Nipponololigo) sumatrensis, Sepia recurvirostra, Sepia madokai, Sepia kobiensis, Amphioctopus aegina, Sepia officinalis, Sepioteuthis lessoniana, Todarodes pacificus, Octopus vulgaris, Heterololigo bleekeri, Octopus sinensis, Octopus americanus, Narrowteuthis nesisi, Ommastrephes bartramii, Sepiella japonica, Uroteuthis (Photololigo) edulis, Doryteuthis (Amerigo) opalescens, Architeuthis dux, Dosidicus gigas, Sepia esculenta, Amphioctopus fangsiao, Loligo vulgaris, Sepiola spp., Octopus mimus, Octopus spp., Octopus bimaculoides, Uroteuthis (Photololigo) chinensis, Uroteuthis (Photololigo) duvaucelii, Illex argentinus, Sepia aculeata, Sepiella inermis, Sepia lycidas, Sepia latimanus, Sepia apama, Sepia pharaonis, Loliolus (Nipponololigo) beka, Alloteuthis subulata, Nototodarus hawaiiensis, Sepia orbignyana, Sepia papuensis, Rossia macrosoma, Lolliguncula (Lolliguncula) brevis, Lolliguncula (Loliolopsis) diomedeae, Afrololigo mercatoris, Octopus bimaculatus, Octopus cyanea, Callistoctopus ornatus, Enteroctopus megalocyathus, Sasakiopus salebrosus, Octopus berrima, Amphioctopus marginatus, Octopus maorum, Octopus fitchi, Amphioctopus neglectus, Loliolus (Nipponololigo) uyii, Loliolus (Nipponololigo) japonica, Bathyteuthis abyssicola, Semirossia patagonica, Cistopus taiwanicus, Sthenoteuthis oualaniensis, Watasenia scintillans, Gonatopsis okutanii, Uroteuthis (Aestuariolus) noctiluca, Sepioteuthis australis, Sepioteuthis sepioidea, Amphioctopus kagoshimensis, Amphioctopus membranaceus, Amphioctopus exannulatus, Amphioctopus rex and Sepia peterseni.

9. The method of claim 1, wherein the identified species of the family of Gastropoda are selected from Helixpomatia, Achatina fulica, Helix aspersa, Helix aspersa maxima, Helix thessalica, Helix lucorum, Helix nicaeensis, Achatina reticulata, Helix aperta, Helix albescens, Tyrrhenaria ceratina, Helix vladika, Helix spp., Pleurodonte discolor, Pleurodonte lychnuchus, Erctella mazzullii, Erctella cephalaeditana, Pleurodonte formosa, Helix christophi, Helix nordmanni, Pleurodonte nucleola, Pleurodonte parilis, Gonostomopsis auridens, Caracolus caracollus, Lacteoluna selenina, Cernuella cisalpine, Cochlicella acuta, Disculella maderensis, Dialeuca nemoraloides, Monadenia fidelis, Cepaea nemoralis, Sphincterochila candidissima, Microphysula ingersolli, Helicodonta obvoluta and Cernuella virgata.

10. The method of claim 1, wherein the identified species of the family of Veneridae are selected from Tridacna mbalavuana, Siliqua alta, Megangulus zyonoensis, Megangulus venulosus, Donax faba, Donax cuneatus, Donax kiusiuensis, Mactra quadrangularis, Ensis ensis, Chamelea gallina, Spisula subtruncata, Polititapes rhomboides, Callista chione, Venerupis corrugata, Polititapes aureus, Venus crebrisulca, Mercenaria campechiensis, Antigona lamellaris, Ameghinomya antiqua, Ameghinomya spp., Callista erycina, Venerupis aspera, Paphia philippiana, Venus casina, Ensis spp., Mactra stultorum, Ensis macha, Siliqua minima, Ensis leei, Polititapes durus, Cerastoderma glaucum, Tridacna spp., Donax longissimus, Solen vaginoides, Venus verrucosa, Ezocallista brevisiphonata, Procardium indicum, Cardium maxicostatum, Cardium costatum, Acanthocardia paucicostata, Acanthocardia echinata, Acanthocardia aculeata, Solen spp., Ruditapes philippinarum, Corculum cardissa, Spisula solida, Scrobicularia plana, Mactra spp., Chamelea striatula, Ensis siliqua, Serripes groenlandicus, Tridacna elongatissima, Tridacna rosewateri, Meretrix lamarckii, Meretrix lusoria, Paphia euglypta, Meretrix spp., Acanthocardia tuberculata, Tridacna maxima, Lutraria rhynchaena, Meretrix lyrata, Arctica islandica, Solen strictus, Paratapes undulatus, Paratapes textilis, Paphia amabilis, Solen grandis, Lutraria maxima, Donax vittatus, Donax variegatus, Donax trunculus, Donax semistriatus, Ruditapes decussatus, Cerastoderma edule, Tridacna squamosa, Mactra chinensis and Mercenaria mercenaria.

11. The method of claim 1, wherein the identified species of the family of Ostreidae are selected from Magallana bilineata, Magallana gigas, Crassostrea virginica, Magallana spp., Magallana angulata, Magallana sikamea, Magallana ariakensis, Ostrea denselamellosa, Magallana nippona, Ostrea edulis, Crassostrea spp., Crassostrea tulipa, Ostrea angasi, Magallana belcheri, Crassostrea rhizophorae, Talonostrea talonata, Crassostrea corteziensis, Ostrea spp., Ostrea chilensis, Ostrea algoensis, Ostrea megodon, Saccostrea cuccullata, Saccostrea palmula, Saccostrea malabonensis, Saccostrea scyphophilla, Saccostrea kegaki, and Saccostrea spp.

12. The method of claim 1, wherein the identified species of the family of Pectinidae are selected from Euvola spp., Mimachlamys crassicostata, Gloripallium pallium, Flexopecten glaber, Aequipecten opercularis, Nodipecten nodosus, Scaeochlamys livida, Pecten spp., Talochlamys multistriata, Patinopecten caurinus, Chlamys behringiana, Placopecten septemradiatus, Pecten maximus, Zygochlamys delicatula, Chlamys hastata, Ylistrum japonicum, Talochlamys gemmulata, Zygochlamys patagonica, Argopecten purpuratus, Argopecten irradians, Azumapecten farreri, Mizuhopecten yessoensi, Placopecten magellanicus, Chlamys islandica, Argopecten ventricosus, Mimachlamys varia, Amusium pleuronectes, Mimachlamys sanguinea, Talochlamys dichroa, Mimachlamys gloriosa, Mimachlamys cloacata, Mimachlamys asperrima, Annachlamys striatula, Decatopecten radula, Bractechlamys vexillum, Aequipecten glyptus, Scaeochlamys lemniscata, Chlamys rubida, Karnekampia sulcata, Crassadoma gigantea, and Ylistrum balloti.

13. The method of claim 1, wherein the identified species of the family of Mytilidae are selected from Mytilus spp., Perna perna, Mytilus unguiculatus, Perna viridis, Mytilus californianus, Mytilus trossulus, Mytilus galloprovincialis, Mytilus edulis and Perna canaliculus.

14. A kit for identifying seafood species of different origin and processing degree in a food sample, comprising at least 2 primer sets (ps) selected from the group consisting of:
i. ps 1 for identifying seafood species of the family of Crustaceans comprising one or more primer pairs of one forward primer selected from any one of SEQ ID NOs: 1 and 2, and a corresponding reverse primer SEQ ID NO: 15 and/or reverse complement sequences of the primer sequences;
ii. ps 2 for identifying seafood species of the family of Cephalopods comprising one or more primer pairs of one forward primer selected from any one of SEQ ID NOs: 3 to 5, and a corresponding reverse primer SEQ ID NO: 16 and/or reverse complement sequences of the primer sequences;
iii. ps 3 for identifying seafood species of the family of Gastropoda comprising one or more primer pairs of forward primer SEQ ID NO: 6, and one reverse primer selected from any one of SEQ ID NOs: 17 to 18 and/or reverse complement sequences of the primer sequences;
iv. ps 4 for identifying seafood species of the family of Veneridae comprising one or more primer pairs of one forward primer selected from any one of SEQ ID NOs: 7 to 11, and one reverse primer selected from any one of SEQ ID NOs: 19 to 21 and/or reverse complement sequences of the primer sequences;
v. ps 5 for identifying seafood species of the family of Ostreidae comprising forward primer SEQ ID NO: 12 and a corresponding reverse primer SEQ ID NO: 22 and/or reverse complement sequences of the primer sequences;
vi. ps 6 for identifying seafood species of the family of Pectinidae comprising forward primer SEQ ID NO: 13 and a corresponding reverse primer SEQ ID NO: 23 and/or reverse complement sequences of the primer sequences; and
vii. ps 7 for identifying seafood species of the family of Mytilidae comprising one or more primer pairs of forward primer SEQ ID NO: 14, and one reverse primer selected from any one of SEQ ID NOs: 24 to 25 and/or reverse complement sequences of the primer sequences,
optionally further comprising PCR components, buffers, reagents and/or an instruction manual.

15. A library of primer sequences comprising any one of SEQ ID NOs: 1 to 25, or any combination thereof.

16. The method of claim 2, wherein amplifying of fragments of DNA in step b) is performed by a PCR comprising 25 cycles at an annealing temperature of 62° C.

17. The method of claim 4, wherein the amplified DNA fragments comprise 120 bp-220 bp of the 16S rDNA.


SEQUENCE LISTING (1153 sequences)

SEQ ID NO: 1 - DNA, 23 bp, synthetic construct
  misc_feature 1..23: note = Primer
  source 1..23: mol_type = other DNA
gggggacgataagaccctataaa

SEQ ID NO: 2 - DNA, 24 bp, synthetic construct
  misc_feature 1..24: note = Primer
  source 1..24: mol_type = other DNA
aatgggaagacaagaccctataaa

SEQ ID NO: 3 - DNA, 21 bp, synthetic construct
  misc_feature 1..21: note = Primer
  source 1..21: mol_type = other DNA
gggacgagaagaccctawtga

SEQ ID NO: 4 - DNA, 21 bp, synthetic construct
  misc_feature 1..21: note = Primer
  source 1..21: mol_type = other DNA
gggacgagaagaccctaatga

SEQ ID NO: 5 - DNA, 21 bp, synthetic construct
  misc_feature 1..21: note = Primer
  source 1..21: mol_type = other DNA
gggacgagaagaccctattga

SEQ ID NO: 6 - DNA, 23 bp, synthetic construct
  misc_feature 1..23: note = Primer
  source 1..23: mol_type = other DNA
tgactgtgcaaaggtagcataat

SEQ ID NO: 7 - DNA, 20 bp, synthetic construct
  misc_feature 1..20: note = Primer
  source 1..20: mol_type = other DNA
ttggcctttaattggggtcc

SEQ ID NO: 8 - DNA, 25 bp, synthetic construct
  misc_feature 1..25: note = Primer
  source 1..25: mol_type = other DNA
ggtagcgcgataatttgtctcttaa

SEQ ID NO: 9 - DNA, 25 bp, synthetic construct
  misc_feature 1..25: note = Primer
  source 1..25: mol_type = other DNA
cttaattggagaagggtatgaatgg

SEQ ID NO: 10 - DNA, 20 bp, synthetic construct
  misc_feature 1..20: note = Primer
  source 1..20: mol_type = other DNA
gtttaacggccgcagttgtc

SEQ ID NO: 11 - DNA, 19 bp, synthetic construct
  misc_feature 1..19: note = Primer
  source 1..19: mol_type = other DNA
taccgcagggataacagcg

SEQ ID NO: 12 - DNA, 21 bp, synthetic construct
  misc_feature 1..21: note = Primer
  source 1..21: mol_type = other DNA
ggtagcgaaattccttgcctt

SEQ ID NO: 13 - DNA, 24 bp, synthetic construct
  misc_feature 1..24: note = Primer
  source 1..24: mol_type = other DNA
tgctaaggtagctaaattatggcc

SEQ ID NO: 14 - DNA, 24 bp, synthetic construct
  misc_feature 1..24: note = Primer
  source 1..24: mol_type = other DNA
ccttttgcataagggtttttcaag

SEQ ID NO: 15 - DNA, 27 bp, synthetic construct
  misc_feature 1..27: note = Primer
  source 1..27: mol_type = other DNA
attacgctgttatccctaaagtaactt

SEQ ID NO: 16 - DNA, 26 bp, synthetic construct
  misc_feature 1..26: note = Primer
  source 1..26: mol_type = other DNA
attacgctgttatccctatggtaact

SEQ ID NO: 17 - DNA, 23 bp, synthetic construct
  misc_feature 1..23: note = Primer
  source 1..23: mol_type = other DNA
aagtttctagggtcttctcgtct

SEQ ID NO: 18 - DNA, 23 bp, synthetic construct
  misc_feature 1..23: note = Primer
  source 1..23: mol_type = other DNA
aaactctaagggtcttctcgtct

SEQ ID NO: 19 - DNA, 22 bp, synthetic construct
  misc_feature 1..22: note = Primer
  source 1..22: mol_type = other DNA
gctcgacagggtcttctcgtct

SEQ ID NO: 20 - DNA, 24 bp, synthetic construct
  misc_feature 1..24: note = Primer
  source 1..24: mol_type = other DNA
caaaaattcaggttttttcacttg

SEQ ID NO: 21 - DNA, 27 bp, synthetic construct
  misc_feature 1..27: note = Primer
  source 1..27: mol_type = other DNA
tcatgtaagaaattaaaaaacgaacag

SEQ ID NO: 22 - DNA, 18 bp, synthetic construct
  misc_feature 1..18: note = Primer
  source 1..18: mol_type = other DNA
aaagttgcacggggtctt

SEQ ID NO: 23 - DNA, 20 bp, synthetic construct
  misc_feature 1..20: note = Primer
  source 1..20: mol_type = other DNA
cttcacggggtcttctcgtc

SEQ ID NO: 24 - DNA, 23 bp, synthetic construct
  misc_feature 1..23: note = Primer
  source 1..23: mol_type = other DNA
cgaatagtatctagccgccattc

SEQ ID NO: 25 - DNA, 25 bp, synthetic construct
  misc_feature 1..25: note = Primer
  source 1..25: mol_type = other DNA
gcaaatagcatatcactttcacctc

SEQ ID NO: 26 - DNA, 33 bp, synthetic construct
  misc_feature 1..33: note = Overhang Forward
  source 1..33: mol_type = other DNA
tcgtcggcagcgtcagatgtgtataagagacag

SEQ ID NO: 27 - DNA, 34 bp, synthetic construct
  misc_feature 1..34: note = Overhang Reverse
  source 1..34: mol_type = other DNA
gtctcgtgggctcggagatgtgtataagagacag

SEQ ID NO: 28 - DNA, 133 bp, Mytilus chilensis
  source 1..133: mol_type = genomic DNA
ccttttgcataagggtttttcaagatcaatttaagaatttaattttcccgaataaaagagagttattttttggagttaggaaatcgtggtagagatttgattaaactaaagaatggcggctagatactattcg

SEQ ID NO: 29 - DNA, 133 bp, Perna perna
  source 1..133: mol_type = genomic DNA
ccttttgcataagggtttttcaagaaaatttacttatgtattttcccgaaaaggaagaagctatcgttgtgagtgtgaataccgtggtaaagttatgaccaactatacgataaaagctagatactaattgcct

SEQ ID NO: 30 - DNA, 133 bp, Mytilus unguiculatus
  source 1..133: mol_type = genomic DNA
ccttttgcataagggtttttcaagataaatttaagcatttaaatttcccgaataaaagagagttattcttttgagttaaaaaatcgtggtagagattaatataaactaaagaatggcggctagatactattcg

SEQ ID NO: 31 - DNA, 132 bp, Perna viridis
  source 1..132: mol_type = genomic DNA
ccttttgtataagggtttttttagaaaatgagtgtataactgtttcccgaaaaaagataagttacttgaatttaatttatttttcgtggtaaagaaaaataaaaaaatcaagtaaaagtgatatgctatttg

SEQ ID NO: 32 - DNA, 133 bp, Mytilus californianus
  source 1..133: mol_type = genomic DNA
ccttttgcataagggtttttcaagataaatttaaatatttaaatttcccgaataaaaaagagttattcctttgagttaaaaaatcgtggtagagatttatataaactaaggaatagcggctagatactattcg

SEQ ID NO: 33 - DNA, 133 bp, Mytilus trossulus
  source 1..133: mol_type = genomic DNA
ccttttgcataagggtttttcaagacaagtttaagaatttaattttcccgaataaaagagagttatttttttgagttaagaaatcgtggcagagatttaattaaactaaagaatagcggctagatactattcg

SEQ ID NO: 34 - DNA, 133 bp, Mytilus galloprovincialis
  source 1..133: mol_type = genomic DNA
ccttttgcataagggtttttcaagataaatttaagaatttaattttcccgaataaaagagagttatttttttgagttaggaaatcgtggtagagatttgattaaactaaagaatggcggctagatactattcg

SEQ ID NO: 35 - DNA, 133 bp, Mytilus edulis
  source 1..133: mol_type = genomic DNA
ccttttgcataagggtttttcaagatcaatttaagaatttaattttcccgaataaaagagagttatttttttgagttaggaaatcgtggtagagatttgattaaactaaagaatggcggctagatactattcg

SEQ ID NO: 36 - DNA, 133 bp, Perna canaliculus
  source 1..133: mol_type = genomic DNA
ccttttgtataagggtttttcaagaaacaaaacgtatgtttgtatcccgaaaaaaataaaagttactttttttcttatatttaacgtggcaaagttaaattataaaaagaggtgaaagtgatatgctatttgc

SEQ ID NO: 37 - DNA, 133 bp, Mytilus platensis; identical to SEQ ID NO: 28
  source 1..133: mol_type = genomic DNA

SEQ ID NO: 38 - DNA, 146 bp, Magallana bilineata
  source 1..146: mol_type = genomic DNA
ggtagcgaaattccttgccttttgattgtaggcctgcatgaatggtttaacgagggtttagctgtctctaaatttttaattgaaattgtattgaaggtgaagataccttcatttaaaagttagacaaaaagaccccgtgcaacttt

SEQ ID NO: 39 - DNA, 146 bp, Magallana gigas
  source 1..146: mol_type = genomic DNA
ggtagcgaaattccttgccttttgattgtgggcctgcatgaatggtttaacgagggtttgactgtctctaaattttttattgaaattgtactgaaggtgaagataccttcatttaaaagttagacaaaaagaccccgtgcaacttt

SEQ ID NO: 40 - DNA, 146 bp, Crassostrea virginica
  source 1..146: mol_type = genomic DNA
ggtagcgaaattccttgccttttgattgtgggcctgcatgaatggtttgacgagggctttgctgtctcttgattttttattgaaattgtagtgtaggtgaaaataccttcataagaaagttagacaagaagaccccgtgcaacttt

SEQ ID NO: 41 - DNA, 146 bp, Magallana hongkongensis
  source 1..146: mol_type = genomic DNA
ggtagcgaaattccttgccttttgattgtaggcctgcatgaatggtttaacgagggtttaactgtctctaaattttttattgaaattgtactgaaggtgaagataccttcatttaaaagttagacaaaaagaccccgtgcaacttt

SEQ ID NO: 42 - DNA, 146 bp, Magallana angulata
  source 1..146: mol_type = genomic DNA
ggtagcgaaattccttgccttttgattgtgggcctgcatgaatggtttaacgagggtttaactgtctctaaattttttattgaaattgtactgaaggtgaagataccttcatttaaaagttagacaaaaagaccccgtgcaacttt

SEQ ID NO: 43 - DNA, 146 bp, Magallana sikamea
  source 1..146: mol_type = genomic DNA
ggtagcgaaattccttgccttttgattgtaggcctgcatgaatggtttaacgagggtttaactgtctctaaattttttattgaaattgtactgaaggtgaagataccttcattaaaaagttagacaaaaagaccccgtgcaacttt

SEQ ID NO: 44 - DNA, 146 bp, Magallana ariakensis
  source 1..146: mol_type = genomic DNA
ggtagcgaaattccttgccttttgattgtaggcctgcatgaatggtttaacgagggtttaactgtctcttgatttaaaattgaaattgtactgaaggtgaagataccttcattaaaaagtcagacaaaaagaccccgtgcaacttt

SEQ ID NO: 45 - DNA, 147 bp, Ostrea denselamellosa
  source 1..147: mol_type = genomic DNA
ggtagcgaaattccttgccttttaattgtaggcctgcatgaatggcttaacgagggcttaactgtctctggtttacagggtctaaattggattaaaggtgaagatacctttatataaaagtcagacaagaagaccccgtgcaacttt

SEQ ID NO: 46 - DNA, 146 bp, Magallana nippona
  source 1..146: mol_type = genomic DNA
ggtagcgaaattccttgccttttgattgtgggcctgcatgaatggtttaacgagggtttgactgtctctaagttttttattgaaattgtattgaaggtgaagataccttcatttaaaagttagacaaaaagaccccgtgcaacttt

SEQ ID NO: 47 - DNA, 146 bp, Ostrea edulis
  source 1..146: mol_type = genomic DNA
ggtagcgaaattccttgccttttaattgtaggcctgcatgaatggtttgacgagggcttaactgtctctagtttgtaagtctaaattggattaaaggtgaagatacctttataaaaaagtcagacaagaagaccccgtgcaacttt

SEQ ID NO: 48 - DNA, 147 bp, Ostrea lurida
  source 1..147: mol_type = genomic DNA
ggtagcgaaattccttgccttttaattgtaggcctgcatgaatggcttgacgagggtttaactgtctctggtttgtaaggtctaaattggattaaaggtgaagatacctttatataaaagtcagacaagaagaccccgtgcaacttt

SEQ ID NO: 49 - DNA, 146 bp, Crassostrea tulipa
  source 1..146: mol_type = genomic DNA
ggtagcgaaattccttgccttttaattgtaggccagcatgaatggtttgacgagggcctcactgtctcttagttctatgttgaaattgtagtgtaggtgaagataccttcataaaaaagtaagacaaaaagaccccgtgcaacttt

SEQ ID NO: 50 - DNA, 146 bp, Ostrea angasi
  source 1..146: mol_type = genomic DNA
ggtagcgaaattccttgccttttaattgtaggcctgcatgaatggtttgacgagggcttaactgtctctagtttgtaagtctaaattggactaaaggtgaagatacctttataaaaaagtcagacaagaagaccccgtgcaacttt

SEQ ID NO: 51 - DNA, 146 bp, Magallana belcheri
  source 1..146: mol_type = genomic DNA
ggtagcgaaattccttgccttttgattgtaggcctgcatgaatggtttaacgagggtttaactgtctctaaatttttagttgaaattgtattgaaggtgaagataccttcattaagaagttagacaaaaagaccccgtgcaacttt

SEQ ID NO: 52 - DNA, 146 bp, Crassostrea rhizophorae
  source 1..146: mol_type = genomic DNA
ggtagcgaaattccttgccttttgattgtgggccagcatgaatggtttgacgagggctttgctgtctctcgattttttattgaaattgtagtgtaggtgaaaataccttcataagaaagttagacaagaagaccccgtgcaacttt

SEQ ID NO: 53 - DNA, 146 bp, Crassostrea brasiliana; identical to SEQ ID NO: 49
  source 1..146: mol_type = genomic DNA

SEQ ID NO: 54 - DNA, 147 bp, Talonostrea talonata
  source 1..147: mol_type = genomic DNA
ggtagcgaaattccttgccttttaattgtaggcctgcatgaatggtttaacgagggttaagctgtctctaagtttataagttgaaattgtattaaaggtgaagatacctttattttgaagttagacaaaaagaccccgtgcaacttt

SEQ ID NO: 55 - DNA, 146 bp, Crassostrea corteziensis
  source 1..146: mol_type = genomic DNA
ggtagcgaaattccttgccttttaattgtaggccagcatgaatggtttgacgagggcctcactgtctctcagctctatattgaaattgcagtggaggtgaagatacctccataagaaagtaagacaagaagaccccgtgcaacttt

SEQ ID NO: 56 - DNA, 146 bp, Magallana rivularis; identical to SEQ ID NO: 41
  source 1..146: mol_type = genomic DNA

SEQ ID NO: 57 - DNA, 147 bp, Ostrea angelica; identical to SEQ ID NO: 48
  source 1..147: mol_type = genomic DNA

SEQ ID NO: 58 - DNA, 147 bp, Ostrea permollis
  source 1..147: mol_type = genomic DNA
ggtagcgaaattccttgccttttaattgtaggcctgcatgaatggcttgacgagggtttaactgtctctggtttgtaaagtctaaattggattaaaggtgaagatacctttatataaaagtcagacaagaagaccccgtgcaacttt

SEQ ID NO: 59 - DNA, 147 bp, Ostrea chilensis
  source 1..147: mol_type = genomic DNA
ggtagcgaaattccttgccttttaattgtaggcctgcatgaatggcttgacgagggtttaactgtctctggtttataaagtctaaattggattaaaggtgaagatacctttatacaaaagtcagacaagaagaccccgtgcaacttt

SEQ ID NO: 60 - DNA, 146 bp, Ostrea algoensis
  source 1..146: mol_type = genomic DNA
ggtagcgaaattccttgccttttaattgtaggcctgcatgaatggcttgacgagggcttaactgtctctagttttaaagtctaaattggattaaaggtgaagatacctttataaaaaagtcagacaagaagaccccgtgcaacttt

SEQ ID NO: 61 - DNA, 147 bp, Ostrea puelchana; identical to SEQ ID NO: 58
  source 1..147: mol_type = genomic DNA

SEQ ID NO: 62 - DNA, 147 bp, Ostrea megodon
  source 1..147: mol_type = genomic DNA
ggtagcgaaattccttgccttttaattgtaggcctgcatgaatggcttgacgagggtttaactgtctctggtttgtaaggtctaaattggattaaaggtgaagatacctttatatgaaagtcagacaagaagaccccgtgcaacttt

SEQ ID NO: 63 - DNA, 147 bp, Saccostrea cuccullata
  source 1..147: mol_type = genomic DNA
ggtagcgaaattccttgccttttaattgtaggcctgcatgaatggattgacgagggtcaagctgtctcttgccttgaataacgaaattggactaaaggtgaagatacctttatagaaaagttagacaagaagaccccgtgcagcttt

SEQ ID NO: 64 - DNA, 147 bp, Saccostrea palmula
  source 1..147: mol_type = genomic DNA
ggtagcgaaattccttgccttttaattgtaggcctgcatgaatggattgacgagggtcaaactgtctcttgctttgtatagcgaaattggactaaaggtgaagatacctttatataaaagttagacaagaagaccccgtgcagcttt

SEQ ID NO: 65 - DNA, 146 bp, Saccostrea malabonensis
  source 1..146: mol_type = genomic DNA
ggtagcgaaattccttgccttttaattgtaggcctgcatgaatggattgacgagggtcaagctgtctcttgccttgatgacgaaattggactaaaggtgaagatacctttatagaaaagttagacaagaagaccccgtgcagcttt

SEQ ID NO: 66 - DNA, 147 bp, Saccostrea scyphophilla
  source 1..147: mol_type = genomic DNA
ggtagcgaaattccttgccttttaattgtaggcctgcatgaatggattgacgagggccattctgtctcttgccttatgaagtgaaattggattaaaggtgaagatacctttattaaaaagttagacaagaagaccccgtgcagcttt

SEQ ID NO: 67 - DNA, 147 bp, Saccostrea glomerata
  source 1..147: mol_type = genomic DNA
ggtagcgaaattccttgccttttaattgtaggcctgcatgaatggattgacgagggtcaaactgtctcttgctttgtatgacgaaattggactgaaggtgaagataccttcatgtgaaagttagacaagaagaccccgtgcagcttt

SEQ ID NO: 68 - DNA, 147 bp, Saccostrea kegaki
  source 1..147: mol_type = genomic DNA
ggtagcgaaattccttgccttttaattgtaggcctgcatgaatggattgacgagggtcaaactgtctcttgctttgtatgtcgaaattggactgaaggtgaagataccttcatataaaagttagacaagaagaccccgtgcagcttt

SEQ ID NO: 69 - DNA, 147 bp, Saccostrea echinata; identical to SEQ ID NO: 67
  source 1..147: mol_type = genomic DNA

SEQ ID NO: 70 - DNA, 147 bp, Saccostrea mytiloides; identical to SEQ ID NO: 67
  source 1..147: mol_type = genomic DNA

SEQ ID NO: 71 - DNA, 147 bp, Euvola vogdesi
  source 1..147: mol_type = genomic DNA
tgctaaggtagctaaattatggcctattaattgtaggtcctgtgaatggtttgacgagtttccaactgtctctagttttttttggtgaacttgaattggatgtgcaaatgcttccatgggtaagaaagacgagaagaccccgtgaag

SEQ ID NO: 72 - DNA, 147 bp, Mimachlamys crassicostata
  source 1..147: mol_type = genomic DNA
tgctaaggtagctaaattatggcccattaattgtgggtcttgcgaacggcttgacgagttccctactgtctcaaagttgttttggtgaacttgaattggatgtgtaaatgcttccatgagtgagaaagacgagaagaccccgtgaag

SEQ ID NO: 73 - DNA, 147 bp, Gloripallium pallium
  source 1..147: mol_type = genomic DNA
tgctaaggtagctaaattacggcctattaattgtaggtcctgtgaatggtttgacgagcctccaactgtctctagtttattttggtgaacttgaattggatgtgcaaatgcttccataggtaagaaagacgagaagaccccgtgaag

SEQ ID NO: 74 - DNA, 147 bp, Flexopecten glaber
  source 1..147: mol_type = genomic DNA
tgctaaggtagctaagttatggtcttttaattttaggccctgtgaaaggattgacgagcttgagtctgtctctatctaaattttatgaaattgaattttaagtgcaaatgcttaagtggacatgaaagacgagaagaccccgtgaag

SEQ ID NO: 75 - DNA, 147 bp, Pecten jacobaeus; identical to SEQ ID NO: 71
  source 1..147: mol_type = genomic DNA

SEQ ID NO: 76 - DNA, 147 bp, Pecten novaezelandiae; identical to SEQ ID NO: 71
  source 1..147: mol_type = genomic DNA

SEQ ID NO: 77 - DNA, 147 bp, Euvola raveneli; identical to SEQ ID NO: 71
  source 1..147: mol_type = genomic DNA

SEQ ID NO: 78 - DNA, 147 bp, Aequipecten opercularis
  source 1..147: mol_type = genomic DNA
tgctaaggtagctaaattatggccctctaattgggggccctgtgaatggattgacgagcctataactgtctctagctgaattatatgaaattgaattttaagtgcaaatgcttaaatataagtgatagacgagaagaccccgtgaag

SEQ ID NO: 79 - DNA, 147 bp, Euvola perula; identical to SEQ ID NO: 71
  source 1..147: mol_type = genomic DNA

SEQ ID NO: 80 - DNA, 146 bp, Nodipecten nodosus
  source 1..146: mol_type = genomic DNA
tgctaaggtagctaaattatggcctattaattgtaggtcctgtgaatggtttgacgagtcttcaactgtctctagtttttttgatgaacttgaattggatgtgcaaatgcttccatgggtaagaaagacgagaagaccccgtgaag

SEQ ID NO: 81 - DNA, 147 bp, Scaeochlamys livida
  source 1..147: mol_type = genomic DNA
tgctaaggtagctaaattatggcccattaattgtgggtcctgtgaatggtttgacgagtcctcgactgtctcaaggttgttttggtgaacttgaattgaatgtgcaaatgctttcatgggtaagaaagacgagaagaccccgtgaag

SEQ ID NO: 82 - DNA, 147 bp, Pecten keppelianus; identical to SEQ ID NO: 71
  source 1..147: mol_type = genomic DNA

SEQ ID NO: 83 - DNA, 147 bp, Talochlamys multistriata
  source 1..147: mol_type = genomic DNA
tgctaaggtagctaaattatggcctattaattgtaggtcctgtgaatggtttgacgagtcctctactgtctcaaggttgttttggtgaacttgaatggaacgtgcaaatgctttcatgagagagaaagacgagaagaccccgtgaag

SEQ ID NO: 84 - DNA, 147 bp, Patinopecten caurinus
  source 1..147: mol_type = genomic DNA
tgctaaggtagctaaattatggcccattaattgtgggtcctgtgaatggtttgacgagtcctctactgtctcaaggttgttttggtgaacttgaattgaacgtgcaaatgctttcatgggaaagaaagacgagaagaccccgtgaag

SEQ ID NO: 85 - DNA, 147 bp, Chlamys behringiana
  source 1..147: mol_type = genomic DNA
tgctaaggtagctaaattatggcccattaattgtgggtcctgtgaatggtttgacgagtcctcgactgtctcaaggttgttttggtgaacttgaattgaatgtgcaaatgctttcatgagaaagaaagacgagaagaccccgtgaag

SEQ ID NO: 86 - DNA, 147 bp, Placopecten septemradiatus
  source 1..147: mol_type = genomic DNA
tgctaaggtagctaaattatggtctattaattgtaggtcttgtgaatggtttgacgagtcttcgactgtctctaggttgttttggtgaacttgaattggatgtgtaaatgcttccgtgggtgagaaagacgagaagaccccgtgaag

SEQ ID NO: 87 - DNA, 147 bp, Pecten maximus
  source 1..147: mol_type = genomic DNA
tgctaaggtagctaaattatggcctattaattgtaggccctgtgaatggtttgacgagtttccaactgtctctagttttttttggtgaacttgaattggatgtgcaaatgcttccatgggtaagaaagacgagaagaccccgtgaag

SEQ ID NO: 88 - DNA, 147 bp, Pecten albicans; identical to SEQ ID NO: 71
  source 1..147: mol_type = genomic DNA

SEQ ID NO: 89 - DNA, 147 bp, Zygochlamys delicatula
  source 1..147: mol_type = genomic DNA
tgctaaggtagctaaattatggcccattaattgtgggtcctgtgaatggtttgacgagtcctcgactgtctcgagattactttggtgaacttgaattaaacgtgcaaatgcttttatgggtaagaaagacgagaagaccccgtgaag

SEQ ID NO: 90 - DNA, 147 bp, Chlamys hastata
  source 1..147: mol_type = genomic DNA
tgctaaggtagctaaattatggcccattaattgtgggtcctgtgaatggtttgacgagtcctcgactgtctcaaggttgttttggtgaacttgaattgaacgtgcaaatgctttcatgagaaagaaagacgagaagaccccgtgaag

SEQ ID NO: 91 - DNA, 147 bp, Ylistrum japonicum
  source 1..147: mol_type = genomic DNA
tgctaaggtagctaaattatggcctattaattgtaggtcctgtgaatggtttgacgagtctttaactgtctctagtttgttttggtgaaattgaattggatgtgcaaatgcttccgtaggtaagaaagacgagaagaccccgtgaag

SEQ ID NO: 92 - DNA, 147 bp, Pecten fumatus; identical to SEQ ID NO: 71
  source 1..147: mol_type = genomic DNA

SEQ ID NO: 93 - DNA, 147 bp, Talochlamys gemmulata
  source 1..147: mol_type = genomic DNA
tgctaaggtagctaaattatggcccattaattgtgggtcctgtgaatggtttgacgagtcctcgactgtctcaagattactttggtgaacttgaattaaatgtgcaaatgcttttatgggtaagaaagacgagaagaccccgtgaag

SEQ ID NO: 94 - DNA, 147 bp, Zygochlamys patagonica
  source 1..147: mol_type = genomic DNA
tgctaaggtagctaaattatggcccattaattgtgggtcctgtgaatggtttgacgagtcctcgactgtctcgagattactttggtgaacttgaattaaatgtgcaaatgcttttatgggtaagaaagacgagaagaccccgtgaag

SEQ ID NO: 95 - DNA, 148 bp, Argopecten purpuratus
  source 1..148: mol_type = genomic DNA
tgctaaggtagctaagttatggcctagttaattgtaggccctgtgaatggtttgacgagttttcttctgtctctagcttgtttcagtgaacttgaattggatgtgcaaatgcttccttgcgaaagaaagacgagaagaccccgtgaag

SEQ ID NO: 96 - DNA, 148 bp, Argopecten irradians
  source 1..148: mol_type = genomic DNA
tgctaaggtagctaagttatggcctagttaattgtaggccctgtgaatggttcgacgagttttcttctgtctctagcttgtttaagtgaacttgaattggatgtgcaaatgcttccttggtaaagaaagacgagaagaccccgtgaag

SEQ ID NO: 97 - DNA, 147 bp, Azumapecten farreri
  source 1..147: mol_type = genomic DNA
tgctaaggtagctaaattatggcctattaattgtaggtcctgtgaatggtttgacgagtcctcgactgtctcgaggttgttttggtgaacttgaattgaatgtgcaaatgctttcatgggaaagaaagacgagaagaccccgtgaag

SEQ ID NO: 98 - DNA, 148 bp, Mizuhopecten yessoensi
  source 1..148: mol_type = genomic DNA
tgctaaggtagctaaattatggcccattaattgtgggtcctgtgaatggtttgacgagtcctctactgtctcaaggttgttttggtgaacttgaattgaatgtgcaaatgctttcatacgaaagaaagacgaagaagaccccgtgaag

SEQ ID NO: 99 - DNA, 147 bp, Placopecten magellanicus
  source 1..147: mol_type = genomic DNA
tgctaaggtagctaagttatggcctattaattgtaggtcttgtgaatggtttgacgagcctccaactgtctctaggttgttttggtgaacttgagttgaatgtgtaaatgccttcgtgggtaagaaagacgagaagaccccgtgaag

SEQ ID NO: 100 - DNA, 147 bp, Euvola ziczac; identical to SEQ ID NO: 71
  source 1..147: mol_type = genomic DNA

SEQ ID NO: 101 - DNA, 147 bp, Pecten sulcicostatus; identical to SEQ ID NO: 71
  source 1..147: mol_type = genomic DNA

SEQ ID NO: 102 - DNA, 147 bp, Chlamys islandica
  source 1..147: mol_type = genomic DNA
tgctaaggtagctaaattatggcccattaattgtgggtcctgtgaatggtttgacgagtcctcgactgtctcaaggttgttttggtgaacttgaattgaatgtgtaaatgctttcatgagaaagaaagacgagaagaccccgtgaag

SEQ ID NO: 103 - DNA, 148 bp, Argopecten ventricosus
  source 1..148: mol_type = genomic DNA
tgctaaggtagctaagttatggcctagttaattgtaggccctgtgaatggtttgacgagttttcctctgtctctagcttgttttagtgaacttgaattggatgtgcaaatgcttccttgagaaagaaagacgagaagaccccgtgaag

SEQ ID NO: 104 - DNA, 147 bp, Mimachlamys varia
  source 1..147: mol_type = genomic DNA
tgctaaggtagctaaattatggcctattaattgtaggtcctgtgaatggtttgacgagtcccctactgtctcgaggttgttttggtgaaattgaattgaacgtgcaaatgctttcatggggaagaaagacgagaagaccccgtgaag

SEQ ID NO: 105 - DNA, 147 bp, Amusium pleuronectes
  source 1..147: mol_type = genomic DNA
tgctaaggtagctaaattatggcctattaattgtaggtcctgtgaatggtttgacgagtttccaactgtctctaattttttttggtgaacttgaattggatgtgcaaatgcttccatgggtaagaaagacgagaagaccccgtgaag

SEQ ID NO: 106 - DNA, 147 bp, Mimachlamys sanguinea
  source 1..147: mol_type = genomic DNA
tgctaaggtagctaaattatggcccattaattgtgggtcctgtgaatggtttgacgagtcctcggctgtctcaaggttgttttggtgaacttgaattgaatgtgcaaatgctttcataggtaagaaagacgagaagaccccgtgaag

SEQ ID NO: 107 - DNA, 147 bp, Talochlamys dichroa
  source 1..147: mol_type = genomic DNA
tgcaaaggtagctaaattatggcccattaattgtgggtcctgtgaacggtttgacgagtcctcgactgtctcaagattactttggtgaacttgaattaaatgtgcaaatgcttttatgggtaagaaagacgagaagaccccgtgaag

SEQ ID NO: 108 - DNA, 147 bp, Mimachlamys gloriosa
  source 1..147: mol_type = genomic DNA
tgctaaggtagctaaattatggcccattaattgtgggtcttgcgaacggcttgacgagttccctactgtctcaaagttgttttggtgaacttgaattggatgtgcaaatgcttccatgagtaagaaagacgagaagaccccgtgaag

SEQ ID NO: 109 - DNA, 148 bp, Mimachlamys cloacata
  source 1..148: mol_type = genomic DNA
tgctaaggtagctgaattctggcccattaaatgtgggtcttagtgaatggtttgacgagtcctcgactgtctcaaggttgtttttttgaaattgaattaaatgtgcaaatgcatttatagggaagaaagacgagaagaccccgtgaag

SEQ ID NO: 110 - DNA, 147 bp, Mimachlamys asperrima
  source 1..147: mol_type = genomic DNA
tgctaaggtagctaaattttggcccattaattgtgggtcttgtgaatggtttgacgagttccctactgtctcaaagttgttttggtgaacttgaattggatgtgcaaatgcatctgtaggtaagaaagacgagaagaccccgtgaag

SEQ ID NO: 111 - DNA, 147 bp, Annachlamys striatula
  source 1..147: mol_type = genomic DNA
tgctaaggtagctaaattatggcctattaattgtaggtcctgtgaatggtttgacgagtcttcaactgtctctgatttgctttggtgaaattgaattggaagtgcaaatgcttccgtaggtaagaaagacgagaagaccccgtgaag

SEQ ID NO: 112 - DNA, 147 bp, Decatopecten radula
  source 1..147: mol_type = genomic DNA
tgcaaaggtagctaaattatggcctattaattgtaggtcctgtgaatggtttgacgagctcccgactgtctctagtttggtttagtgaacttgaattggatgtgcaaatgcttccatgggaaagaaagacgagaagaccccgtgaag

SEQ ID NO: 113 - DNA, 147 bp, Bractechlamys vexillum
  source 1..147: mol_type = genomic DNA
tgcaaaggtagctaaattacggcctattaattgtaggtcctgtgaatggtttgacgagcctccggctgtctctaatctgttttggtgaacttgaattggacgtgcaaatgcttccttgaaaaagaaagacgagaagaccccgtgaag

SEQ ID NO: 114 - DNA, 147 bp, Aequipecten glyptus
  source 1..147: mol_type = genomic DNA
tgctaaggtagctaaattatggcctattaattgtaggtcctgtgaatggtttgacgagtttttaactgtctctggtttgttttggtgaatttgaattggaagtgcaaatgctttcgtgagtgagaaagacgagaagaccccgtgaag

SEQ ID NO: 115 - DNA, 147 bp, Scaeochlamys lemniscata
  source 1..147: mol_type = genomic DNA
tgctaaggtagctaaattatggcctattaattgtaggtcctgtgaatggtttgacgagtcttcaactgtctcaaagttattttggtgaacttgaattaaatgtgcaaatgcatttatagggaagaaagacgagaagaccccgtgaag

SEQ ID NO: 116 - DNA, 147 bp, Chlamys rubida; identical to SEQ ID NO: 85
  source 1..147: mol_type = genomic DNA

SEQ ID NO: 117 - DNA, 147 bp, Karnekampia sulcata
  source 1..147: mol_type = genomic DNA
tgctaaggtagctaaattttggtctattaattgtaggtcttgtgaatggtttgacgagtcttcgactgtctctaggttgttttggtgaacttgaattgaatgtgtaaatgctttcgtgggcgagaaagacgagaagaccccgtgaag

SEQ ID NO: 118 - DNA, 147 bp, Crassadoma gigantea
  source 1..147: mol_type = genomic DNA
tgctaaggtagctaaattatggcccattaattgtgggtcctgtgaatggtttgacgagtcctcgactgtctcaaggttgttttggtgaacttgaattgaacgtgcaaatgctttcatgggagagaaagacgagaagaccccgtgaag

SEQ ID NO: 119 - DNA, 147 bp, Ylistrum balloti
  source 1..147: mol_type = genomic DNA
tgctaaggtagctaaattatggcctattaattgtaggtcctgtgaatggtttgacgagtctttaactgtctctagtttgttttggtgaaattgaattggatgtgcaaatgcttccataggtgagaaagacgagaagaccccgtgaag

SEQ ID NO: 120 - DNA, 143 bp, Tridacnambalavuana
  source 1..143: mol_type = genomic DNA
agtagcgtgataagtcggcctttaattggggtctggtatgaaggggttgacgtcggagaagctgtctcaaaagaattttgtgaaatttactttctagtgaaaaggctagaataatattaaaagacgagaagaccctgtcgagc

SEQ ID NO: 121 - DNA, 144 bp, Siliqua alta
  source 1..144: mol_type = genomic DNA
ggtagcgtaataaattgtctcttaattggagaagggtatgaatggtttgacgtaagggtttctgtctcaacaatagtaatgtgaagttttcttttaagtcaaaaggcttaaattatattaaaagacgagaagaccctgtcgagc

SEQ ID NO: 122 - DNA, 143 bp, Megangulus zyonoensis
  source 1..143: mol_type = genomic DNA
ggtagcataataggtcgtcctttaattgggggaaagtatgaatggtcggatgttggaaattctttattaagagtaggtgttaaattttactttttagtgcaaaggctaagataatattattagacgagaagaccctgttgagc

SEQ ID NO: 123 - DNA, 143 bp, Megangulus venulosus
  source 1..143: mol_type = genomic DNA
ggtagcataataggtcgtcctttaattgggggaaagtatgaatggtcggatgttggaaatcctttattaagagtaggtgttaaattttactttttagtgcaaaggctaaaattagattattagacgagaagaccctgttgagc

SEQ ID NO: 124 - DNA, 150 bp, Donax faba
  source 1..150: mol_type = genomic DNA
gcgttaaagtagcataataggtcgtcttttaattggagaatggaatgaatggagtgacgatggaaaatctttattagaaatataagttgaagtttatttttaagtgaaaaggcttaaattaaggtgttagacgagaagaccctattgagc

SEQ ID NO: 125 - DNA, 150 bp, Donax cuneatus
  source 1..150: mol_type = genomic DNA
gcgttaaggtagcataatagctcgtcttttaattggaggatggaatgaatggagtgacgatggaaagtctttattaaaaacactgatcgaagtttacttttaagtgaaaaggcttaaatggagttgttagacgagaagaccctgttgagc

SEQ ID NO: 126 - DNA, 150 bp, Donax kiusiuensis
  source 1..150: mol_type = genomic DNA
gcgttaaagtagcataataaattgtctcttaattagaggatagtatgaaaggttggatgttggagaagctttattggaagcattaattgaagtttacatataagtgaaaaggcttatatgagattgttagacgagaagaccctgttgagc

SEQ ID NO: 127 - DNA, 137 bp, Mactra quadrangularis
  source 1..137: mol_type = genomic DNA
gcaataaattgtcctttaattggggaaatgaatgaaggggttgacgagggggaagctgtctttttaatatagttcgaagttttcttttatgtgaaaagacttagattttacaataagaagagaagaccccggcgagc

SEQ ID NO: 128 - DNA, 124 bp, Ensis ensis
  source 1..124: mol_type = genomic DNA
cttaattggagaagggtatgaatggtttgacgtggtagtttctgtctcaatggtaatattatgaagttttcttttgagttaaaagtctcaaatttagataaaagacgagaagaccctgtcgagc

SEQ ID NO: 129 - DNA, 149 bp, Chamelea gallina
  source 1..149: mol_type = genomic DNA
gtgctaaggtagcgtgataagttgtcttttaattggagaatggtatgaatggtttaacgtagaataactgtctttggaaaataaaattaagtttccatgtaagtgaaaaggcttatatttttgtaaaagacgagaagaccctgtcgagc

SEQ ID NO: 130 - DNA, 143 bp, Spisula subtruncata
  source 1..143: mol_type = genomic DNA
ggtagcgcagtcactcgtcttttaattggagaaaggtatgaatggtttgacgagggaaaatctgtctcaaggatatgggctaaagttttcttttaagtgaaaagacttaaatttttaaataagaagagaagaccccggcgagc

SEQ ID NO: 131 - DNA, 146 bp, Callista chione
  source 1..146: mol_type = genomic DNA
gtttaacggccgcagttgtcctgtgctaaggtagcataatgattagctctttaattgggggaaagtatgaatggtttgacgtggagatactgtcttaaatataataaatagaaattttttttcaagtgaaaaaacctgaatttttg

SEQ ID NO: 132 - DNA, 150 bp, Polititapes rhomboides
  source 1..150: mol_type = genomic DNA
tgtactaaggtagcgcggtaatttgtcccttaattgggggatggtatgaaaggcttgtcttaagttttttgtctcaaaaagacaaagtgaagtttccttttagatgaaaaggtctaaataaaaataaaagacgagaagaccctgtcgagc

SEQ ID NO: 133 - DNA, 141 bp, Venerupis corrugata
  source 1..141: mol_type = genomic DNA
ggtagcgcgataatttgtctcttaattggagaatggtatgaaaggtttgacgcagatcagttgtctcttagaaaaaaggtgaagtttccttttaaatgagaaggtttaaatttgaataaaagacgagaagaccctgccgag

SEQ ID NO: 134 - DNA, 150 bp, Polititapes aureus
  source 1..150: mol_type = genomic DNA
tgtactaaggtagcgcgataatttgtctcttaattggagaatggtatgaaaggtttgacgcagattgattgtctctagaaaagaaaatgaagtttccttttaaatgaaaaggtttaaattgtaataaaagacgagaagaccctgccgagc

SEQ ID NO: 135 - DNA, 149 bp, Venus crebrisulca
  source 1..149: mol_type = genomic DNA
gtgctaaggtagcgtgataagttgtcctttgattggggaatggtatgaaaggtttgacgtggaataactgtctttagaaaattaaactaagtttccatttaagtgaaaaggcttatatttttgtaaaagacgagaagaccctgtcgagc

SEQ ID NO: 136 - DNA, 142 bp, Mercenaria campechiensis
  source 1..142: mol_type = genomic DNA
ggtagcgtgataagttgtcttttgattggagaatggaatgaagggtttgacgtagaatttctttttttgtagaagatactaaaatttccttctaagtgaaaaggcttaggtttttgaaaaagacgagaagaccctgtcgaac

SEQ ID NO: 137 - DNA, 149 bp, Antigona lamellaris
  source 1..149: mol_type = genomic DNA
gtgctaaggtagcgtgataggttgtctcttaattggagaatggtatgaagggtctcacgtggggtttctttttttaattaataaaatgaagtttctttttaagtgaaaaagcttaagtttttgtaaaagacgagaagaccctgtcgagc

SEQ ID NO: 138 - DNA, 149 bp, Ameghinomya antiqua
  source 1..149: mol_type = genomic DNA
gtgctaaggtagcgtgataagttgtcctttgattggggactggtatgaatggcttgacgtagaatgactatttttatagaataaagtaaactttccttctgagtgaaaaggcttaggtttttgtaaaagacgagaagaccctgtcgagc

SEQ ID NO: 139 - DNA, 151 bp, Callista erycina
  source 1..151: mol_type = genomic DNA
gtttaacggccgcagttgtcctgtgctaaggtagcataataattagctctttaattgggggaaagtatgaatggtttgacgtggaaatactgtctttaatgtaattaagagaaatttttttataggtgaaaaaacctaaatatttgtaaga

SEQ ID NO: 140 - DNA, 142 bp, Venerupis aspera
  source 1..142: mol_type = genomic DNA
ggtagcgcgataatttgtctcttaattggagaatggtatgaaaggtttgtcatagagcggttgtctctaaaaaattaagtgaaatttctttttaaatgaaaaggtttaaataaaaataaaagacgagaagaccctgtcgagc

SEQ ID NO: 141 - DNA, 141 bp, Paphia philippiana
  source 1..141: mol_type = genomic DNA
ggtagcgtgataatttgtcctttaattggggaatggtatgaaaggtttgacgtggatgagttgtctctagaaaaattattgaagtttccttttaaatgaaaaggtttaaatttttgtaaaagacgagaagaccctggcgag

SEQ ID NO: 142 - DNA, 149 bp, Venus casina
  source 1..149: mol_type = genomic DNA
gtgctaaggtagcgtggtaagttgtcttttaattggagaatggtatgaatggtttgacgtagaataactgtctttggggaatagaattaagtttccttttaagtgaaaaggcttatatttttgtaaaagacgagaagaccctgtcgagc

SEQ ID NO: 143 - DNA, 124 bp, Ensis terranovensis; identical to SEQ ID NO: 128
  source 1..124: mol_type = genomic DNA

SEQ ID NO: 144 - DNA, 142 bp, Mactra stultorum
  source 1..142: mol_type = genomic DNA
ggtagcgcaatcaattgtcctttaattggggaaaggaatgaaggggttgacgagggtgagctgtctctgtagtatagcttaaacttttcttttaagtgaaaagacttaaattttaaaataagacgagaagaccccggcgagc

SEQ ID NO: 145 - DNA, 124 bp, Ensis macha
  source 1..124: mol_type = genomic DNA
cttaattggagaagggtatgaatggtttgacgtggtagtttctgtctcaatgatagtattacgaagttttcctttgagttaaaagtctcaaataaatgtaaaagacgagaagaccctgtcgagc

SEQ ID NO: 146 - DNA, 144 bp, Siliqua minima
  source 1..144: mol_type = genomic DNA
ggtagcgtaataaattgtctcttaattggagaagggtatgaatggtttgacgtaggagtttctgtctcaacaatagtaatgcgaagttttcctttaagtaaaaaggcttaaatattattaaaagacgagaagaccctgtcgagc

SEQ ID NO: 147 - DNA, 124 bp, Ensis leei; identical to SEQ ID NO: 128
  source 1..124: mol_type = genomic DNA

SEQ ID NO: 148 - DNA, 136 bp, Polititapes durus
  source 1..136: mol_type = genomic DNA
ggtagcgcgataatttgtctcttaattggagaatggaatgaatggtttgtcgcatgttggtgtctctgggaaaatgaagtttccttttaattgaaaaggtttaaattttaataaaagacgagaagaccctgtgaga

SEQ ID NO: 149 - DNA, 128 bp, Cerastoderma glaucum
  source 1..128: mol_type = genomic DNA
ttggcctttaattggggtccggtatgaatgggttgacgatagaagctctgtctcgttagaatgtgttgaaatttacatttcggtgaaaagaccgaaccgcttctaaaagacgagaagaccccgtcgag

SEQ ID NO: 150 - DNA, 149 bp, Donax longissimus
  source 1..149: mol_type = genomic DNA
gtactaaggtagcgctacaatttgccctttaaatgggggctggtatgaatgggttcacgtgagagttgctgtgtcacaaaaatttattgaaattaacttttaggtgaagaggcctagctgagtttaaaggacgacaagaccctagaagc

SEQ ID NO: 151 - DNA, 124 bp, Solen vaginoides
  source 1..124: mol_type = genomic DNA
cttaattgggggctagtatgaagggtttgacgtaaaaaaagttgtctctgagataatattgtgaagttttttttctggtgaaaaatctaggatttaattaaaagacgagaagaccctgtcgagc

SEQ ID NO: 152 - DNA, 149 bp, Venus verrucosa
  source 1..149: mol_type = genomic DNA
gtgctaaggtagcgtgataagttgtcttttaattggagaatggtatgaatggtttgacgtagaataactgtctttggaaaattaaactaagtttccttttaagtgaaaaggcttatatttttgtaaaagacgagaagaccctgtcgagc

SEQ ID NO: 153 - DNA, 146 bp, Ezocallista brevisiphonata
  source 1..146: mol_type = genomic DNA
atttaaaggctgcagttatctgtacaaaggtagcgcaataagtagccccttaattgggggatggtatgaatggtttgacgtggagatgctgtttctagtgtagtttaaatgaaatttctttctaggtgaaaaagcctaggtttttg

SEQ ID NO: 154 - DNA, 141 bp, Procardium indicum
  source 1..141: mol_type = genomic DNA
agtagcgtaataatttgtcccttaattagggtctgtatgaacgggttgacgtggaaaaactgtcttgaggaaatagtttgaaattttctttttagtgaaaagcctgaaatagtattaaaagacgagaagaccccgtcgagc

SEQ ID NO: 155 - DNA, 141 bp, Cardium maxicostatum
  source 1..141: mol_type = genomic DNA
agtagcgtaataatttgtcccttaattagggtctgtatgaaagggttgacgtggaaaaactgtcttgaaaaaataatctaaaattttctttttagtgaaaagcctgaaatgtgcttaaaagacgagaagaccccgtcgagc

SEQ ID NO: 156 - DNA, 141 bp, Cardium costatum
  source 1..141: mol_type = genomic DNA
agtagcgtaataatttgtcccttaattagggtctgtatgaaagggttgacgtggaagaactgtcttgaaaaaataatctaaaattttctttttagtgaaaagcctaagatatatttaaaagacgagaagaccccgtcgagc

SEQ ID NO: 157 - DNA, 129 bp, Acanthocardia paucicostata
  source 1..129: mol_type = genomic DNA
ttggcctttaattggggtccagtatgaatgggttgacgatagaatggctgtcttataggggggccttagaaattttctttttggtgaaaagacctaaataagtttaatagacgagaagaccccgtcgag

SEQ ID NO: 158 - DNA, 129 bp, Acanthocardia echinata
  source 1..129: mol_type = genomic DNA
ttggcctttaattggggtccagtatgaatgggttgacgatagaatagctgtcttacaggggttcattagaaattttctttttggtgaaaagacctgattaaatttaatagacgagaagaccccgtcgag

SEQ ID NO: 159 - DNA, 129 bp, Acanthocardia aculeata
  source 1..129: mol_type = genomic DNA
ttggcctttaattggggtccagtatgaatgggttgacgatagaatagctgtcttataggggcccattagaaattttctttttggtgaaaagacctgattaaacttaatagacgagaagaccccgtcgag

SEQ ID NO: 160 - DNA, 141 bp, Ruditapes philippinarum
  source 1..141: mol_type = genomic DNA
ggtagcgcgataatttgtctcttaattagagaatggtatgaagggttagacgtgggattattgtctctaaaaaatacagttaagtttccttttaaatgaaaaggtttaaatttattaaaagacgagaagaccctgtcgagc

SEQ ID NO: 161 - DNA, 143 bp, Corculum cardissa
  source 1..143: mol_type = genomic DNA
agtagcataataatttgccctttaattgggggatagtatgaatgggttgacgatggaaagactgtctccagaagagtaattgaaattttcttcccagtgaaaagcctgggattaggttgaaagacgagaagaccccgtcgagc

SEQ ID NO: 162 - DNA, 143 bp, Spisula solida
  source 1..143: mol_type = genomic DNA
ggtagcgcaatcaatcgtctcttaattggagaaaggtatgaatggtttgacgagggaaaaactgtctcaaatatagattttgaagttttcttttaagtgaaaagacttaaatttttaaataagaagagaagaccccggtgagc

SEQ ID NO: 163 - DNA, 143 bp, Scrobicularia plana
  source 1..143: mol_type = genomic DNA
ggtagcgcgataggtcgctctttaattgggagagagtatgaatggtcggatgttggaaagcctttattaaaagtaattattgaaatttaccttttagtgcaaaggctaaaatgattttattagacgagaagaccctgttgagc

SEQ ID NO: 164 - DNA, 143 bp, Mactra violacea
  source 1..143: mol_type = genomic DNA
ggtagcgcagtcaattgtcctttaattggggaaaggaatgaaagggttgacgagggggaggctgtctttgtaatataatttgaaattttcttttacgtgaaaagacttaaattttaaaataagaagagaagaccccggcgagc

SEQ ID NO: 165 - DNA, 149 bp, Chamelea striatula
  source 1..149: mol_type = genomic DNA
gtgctaaggtagcgcggtaagttgtcttttaattggagaatggtatgaatggtttaacgtagaataactgtctttgaggaatagagttaaatttccatttaagtgaaaaggcttatatttttgtaaaagacgagaagaccctatcgaac

SEQ ID NO: 166 - DNA, 124 bp, Ensis siliqua
  source 1..124: mol_type = genomic DNA
cttaattggagaagggtatgaatggtttgacgtggtagtttctgtctcaataatagtattgcgaaattttcatttaagttaaaagtcttaaatacagataaaagacgagaagaccctgtcgagc

SEQ ID NO: 167 - DNA, 142 bp, Serripes groenlandicus
  source 1..142: mol_type = genomic DNA
agtagcgtaataatttggcccttaattagggtctggaatgaatgggttgacgtgggagggctgtctcaaagggattacttaaaattttcttcctagtgaaaagcctgggatactagtaaaagacgagaagaccccgtcgagc

SEQ ID NO: 168 - DNA, 143 bp, Tridacna elongatissima
  source 1..143: mol_type = genomic DNA
agtagcgtgataagtcggcctttaattggggtctggtatgaaggggttgacgtcggaaaagctgtctcggaaaaactaagtgaaatttacttttaagtgaaaaggcttaaataaggataaaagacgagaaggccctgtcgagc

SEQ ID NO: 169 - DNA, 143 bp, Tridacna rosewateri
  source 1..143: mol_type = genomic DNA
agtagcgtgataagtcggcctttaattggggtctggtatgaaggggttgacgtcggaaaagctgtctcagaaaaactaagtgaaatttacttttaagtgaaaaggcttaaatgggggtaaaagacgagaaggccctgtcgagc

SEQ ID NO: 170 - DNA, 140 bp, Meretrix lamarckii
  source 1..140: mol_type = genomic DNA
taccgcagggataacagcgtaatttttttccgtaagttgtattgggaaaaaagtttacaacctcgatgttgaattaagaaaactttgtggcgcagacgccacagtgagtaaaactgttcgttttttaatctcttacatga

SEQ ID NO: 171 - DNA, 138 bp, Meretrix lusoria
  source 1..138: mol_type = genomic DNA
taccgcagggataacagcgtaatttttttttgtaattgtatcgataaaagagtttacgacctcgatgttgaattaagaaacctttatggcgcaggagctataggaggaaaactgttcgttttttaatttcttacatga

SEQ ID NO: 172 - DNA, 142 bp, Paphia euglypta
  source 1..142: mol_type = genomic DNA
ggtagcgcgataatttgtctcttaattggagaatggtatgaaaggtttgacgtagacctattgtctctaaagaaaaaaatgaagtttccttttaaatgaaaaggtttaaatttttgtaaaagacgagaagaccctgccgagc

SEQ ID NO: 173 - DNA, 138 bp, Meretrix meretrix
  source 1..138: mol_type = genomic DNA
taccgcagggataacagcgtaatttttttctgtaattgtatcgagaaaagagtttacgacctcgatgttgaattaagaaacctttatggcgcaggagctataggaggaaaactgttcgttttttaatttcttacatga

SEQ ID NO: 174 - DNA, 138 bp, Meretrix petechialis; identical to SEQ ID NO: 173
  source 1..138: mol_type = genomic DNA

SEQ ID NO: 175 - DNA, 129 bp, Acanthocardia tuberculata
  source 1..129: mol_type = genomic DNA
ttggcctttaattggggtccagtatgaatgggttgacgatggaatagctgtcttataggggctcattagaaattttctttttggtgaaaagacctaattaaacttaatagacgagaagaccccgtcgag

SEQ ID NO: 176 - DNA, 143 bp, Tridacna maxima
  source 1..143: mol_type = genomic DNA
agtagcgtgataagtcggcctttaattggggtctggtatgaaagggttgacgtcggaaaagctgtctcagaaagattaaatgaaatttacttttaagtgaaaaggcttagatgggagtaaaagacgagaaggccctgtcgagc

SEQ ID NO: 177 - DNA, 143 bp, Lutraria rhynchaena
  source 1..143: mol_type = genomic DNA
tgtagcgtaataattagccttttaaaaggagggaggaatgaatggtttgacgagagacctgctgtctcggaagtaaattctaaagttttcttttaagtgaaaagacttagatttttaaataagaagagaagaccccggtgagc

SEQ ID NO: 178 - DNA, 140 bp, Meretrix lyrata
  source 1..140: mol_type = genomic DNA
taccgcagggataacagcgtaatttttttttgtaaattttattggaaaaaaagtttacgacctcgatgttgaattaagaaaactttatggcttaggcgctataatgagtaaaactgttcgttttttaatttcttacatga

SEQ ID NO: 179 - DNA, 143 bp, Arctica islandica
  source 1..143: mol_type = genomic DNA
ggtagcgtaatcagttgtcctttaattggggaaaggaatgaatggtttgacgaaagggtagctgtttcagaggtagataaggaagttttctttcaggtgaaaagacctgagtttttgtaaaagacgagaagaccccgtcgaac

SEQ ID NO: 180 - DNA, 124 bp, Solen strictus
  source 1..124: mol_type = genomic DNA
cttaattgggggctagaatgaagggtttgacgtaaaaaaatttgtctccaaaatagtagtatgaagttttcttttcagtgaaaagcctgaaattgtgataaaagacgagaagaccctgtcgagc

SEQ ID NO: 181 - DNA, 141 bp, Paratapes undulatus
  source 1..141: mol_type = genomic DNA
ggtagcgcgataatttgtctcttaattggagaatggtatgaaaggtttgtcgcagactggttgtctctgaagaaatagatgaagtttccttttaaatgaaaaggtttaaatttttgtaaaagacgagaagaccctgccgag

SEQ ID NO: 182 - DNA, 141 bp, Paratapes textilis
  source 1..141: mol_type = genomic DNA
ggtagcgcgataatttgtctcttaattggagaatggtatgaaaggtttgtcgcagattaattgtctctaaaaaaaacaatgaagtttcctttcaaatgaaaaggtttaaatttttgtaaaagacgagaagaccctgtcgag

SEQ ID NO: 183 - DNA, 143 bp, Paphia amabilis
  source 1..143: mol_type = genomic DNA
ggtagcgcgataatttgtctcttaattggagaatggtatgaaaggtttgacgtagatcgattgtctctataaaaaaaaaatgaagtttccttttaaatgaaaaggtttaaatttttgtaaaagacgagaagaccctgccgagc

SEQ ID NO: 184 - DNA, 124 bp, Solen grandis
  source 1..124: mol_type = genomic DNA
cttaattgggggctagaatgaagggtttgacgtaaaaaagtttgtctccaaaataaaagtatgaagttttcttttcagtgaaaagtctgatataatggtaaaagacgagaagaccctgtcgagc

SEQ ID NO: 185 - DNA, 143 bp, Lutraria maxima
  source 1..143: mol_type = genomic DNA
tgtagcgtaatcattagccttttaaaaggagggaggaatgaatggtttgacgagggacctgctgtctcaggagtaatttctaaagttttcttttaagtgaaaagacttaaatttttaaataagaagagaagaccccggtgagc

SEQ ID NO: 186 - DNA, 151 bp, Donax vittatus
  source 1..151: mol_type = genomic DNA
gtattgaggtagcataataattagtcttttaattgggggatggaatgaatggttgaatgatggggtgtctttattagtagtataaattcgaagttttcttttaagtgaaaagacttaaatttttttgttagacgataagaccctattgagc

SEQ ID NO: 187 - DNA, 151 bp, Donax variegatus
  source 1..151: mol_type = genomic DNA
gcgttacggtagcataataagttgtcttttaattgggggatagtatgaatggtttgatgatggggtagctttattaataatatacatgtgaagtttacttttaagtgaaaaggcttaagttgttttgttagacgatgagaccctatcgagc

SEQ ID NO: 188 - DNA, 151 bp, Donax trunculus
  source 1..151: mol_type = genomic DNA
gtgttaaggtagcataataaatagtctcttaattgggggatagaatgaatggttgaatgatgggatagctttattagtaatataaatatgaagtttacttttaagtgaaaaggcttaaattttattgttagacgataagaccctattgagc

SEQ ID NO: 189 - DNA, 151 bp, Donax semistriatus
  source 1..151: mol_type = genomic DNA
gtgttaaggtagcataataattagtcttttaattgggggatggaatgaatggttgaatgatggggtgtctttattagtagtataaatttgaagttttcttttaagtgaaaaggcttaaatttttttgttagacgataagaccctattgagc

SEQ ID NO: 190 - DNA, 140 bp, Ruditapes decussatus
  source 1..140: mol_type = genomic DNA
ggtagcgcgataatttgtcttttaattgaagaatggtagaaaggttgtcgcaagttgcttgtctctgaaaaagaaagcgaagtttccttttaaatgaaaaggtttaagtaaagataaaagacgagaagaccctattgagc

SEQ ID NO: 191 - DNA, 128 bp, Cerastoderma edule
  source 1..128: mol_type = genomic DNA
ttggcctttaattggggtccggtatgaatgggttgacgatagaaaagctgtctcattggaatatattgaacttttcttctcggtgaaaagaccgcgcttctgttaaaagacgagaagaccccgtcgag

SEQ ID NO: 192 - DNA, 143 bp, Tridacna squamosa
  source 1..143: mol_type = genomic DNA
agtagcgtgataagtcggcctttaattggggtctggtatgaaagggttgacgtcggaaaaactgtctcggaagaactaagtgaaatttacttttaagtgaaaaggcttaaattaaagtaaaagacgagaagaccctgtcgagc

SEQ ID NO: 193 - DNA, 141 bp, Ruditapes philippinarum
  source 1..141: mol_type = genomic DNA
ggtagcgcgataatttgtctcttaattagagaatggtatgaagggttagacgtgggattattgtctctaaaaaakakagttaagtttccttttaaatgaaaaggtttaaatttattaaaagacgagaagaccctgtcgagc

SEQ ID NO: 194 - DNA, 143 bp, Mactra chinensis
  source 1..143: mol_type = genomic DNA
ggtagcgcaataaattgtcctttaattggggaaatgaatgaaagggttgacgagggggaagctatctttttggtatagtttgaagttttcttttatgtgaaaagacttagattttataattagaagagaagaccccggcgagc

SEQ ID NO: 195 - DNA, 143 bp, Tridacna derasa
  source 1..143: mol_type = genomic DNA
agtagcgtgataagtcggcctttaattggggtctggtatgaaggggttgacgtcggagaaactgtctcggaaaaatatggtgaaatttacttttaagtgaaaaggcttaaataaaagtaaaagacgagaaggccctgtcgagc

SEQ ID NO: 196 - DNA, 142 bp, Mercenaria mercenaria
  source 1..142: mol_type = genomic DNA
ggtagcgtgataagttgtcttttaattggagaatggaatgaagggtttgacgtgaaatttctttttttataaaaggtactaaagtttccttctaagtgaaaaggcttaggtttttgaaaaagacgagaagaccctgtcgaac

SEQ ID NO: 197 - DNA, 143 bp, Tridacna gigas
  source 1..143: mol_type = genomic DNA
agtagcgtgataagttggcctttaattggggtctggtatgaaggggttgacgtcggagaaactgtctcggaaagataaaatgaaatttacgtcctagtgaaaaggctgggatggctgtaaaagacgagaagaccctgtcgagc

SEQ ID NO: 198 - DNA, 143 bp, Mactra antiquata; identical to SEQ ID NO: 164
  source 1..143: mol_type = genomic DNA

SEQ ID NO: 199 - DNA, 155 bp, Helix pomatia
  source 1..155: mol_type = genomic DNA
tgactgtgcaaaggtagcataatcagttggcttttaattagagtctggtatgaacgaaaatatgggtagtaactgtcttagcacattttttcgaacttattaaccaagtgcaaatgcttgtaatttaacaatagacgagaagaccctagaaactt

SEQ ID NO: 200 - DNA, 157 bp, Achatina fulica
  source 1..157: mol_type = genomic DNA
tgactgtgcaaaggtagcataataatttgtcctctaattaaggtctggaatgaagggggacacaggggagagctgtctccaataaggttaatttaactttcttatcaggtgaaaattcctgagcctccgatgaaagacgagaagacccttagagttt

SEQ ID NO: 201 - DNA, 155 bp, Helix aspersa
  source 1..155: mol_type = genomic DNA
tgactgtgcaaaggtagcataatcagttggcctataattgaggtctggtatgaacgaataattgggtagtaactgtcttaatgtattcttacaaatttagtaagcaagtgaaaatacttgctaaattataaaagacgagaagaccctagaaactt

SEQ ID NO: 202 - DNA, 155 bp, Helix thessalica
  source 1..155: mol_type = genomic DNA
tgactgtgcaaaggtagcataatcagttggcttttaattagagtctggtatgaacgaaaatatgggtagtaactgtcttagcacatttctttgaacttgttaaacaagtgcaaatgcttgtaattaaaaaatagacgagaagaccctagaaactt

SEQ ID NO: 203 - DNA, 155 bp, Helix pomatia
  source 1..155: mol_type = genomic DNA
tgactgtgcaaaggtagcataatcagttggcttttaattagagtctggtatgaacgaaaatatgggtagtaactgtcttagcatattttttcgaacttattaaacaagtgcaaatgcttgtaatttaacaatagacgagaagaccctagaaactt

SEQ ID NO: 204 - DNA, 155 bp, Helix lucorum
  source 1..155: mol_type = genomic DNA
tgactgtgcaaaggtagcataatcagttggcttttaattggagtctggtatgaacgaacacatgggtggcgactgtctaaatatatttttcctaactttttaatcaagtgaaaatacttgtgctaataaaaaagacgagaagaccctagaaactt

SEQ ID NO: 205 - DNA, 156 bp, Helix nicaeensis
  source 1..156: mol_type = genomic DNA
tgactgtgcaaaggtagcataatcagttggcttttaattggagtctggcatgaacgaaagcatgggtggcgactgtcttaatatattttttctaattttttaatcaagtgaaaatacttgtgtaaaataaaaaagacgagaagaccctagaaactt

SEQ ID NO: 206 - DNA, 157 bp, Achatina reticulata
  source 1..157: mol_type = genomic DNA
tgactgtgcaaaggtagcataataatttgtcctttaatttaggtctggaatgaatgagaacacaggggagagctgtctctagwarggttaacttaacttccttatcaggtgaaaattcctgagcttctttggaaagacgagaagacccttagagttt

SEQ ID NO: 207 - DNA, 154 bp, Helix aspersa maxima
  source 1..154: mol_type = genomic DNA
gactgtgcaaaggtagcataatcagttggcctataattgaggtctggaatgaacgaaaaatagggtagcaactgtcttagtatatttttttaaacttagttagcaagtgaaaatacttgcaaatatacaaaagacgagaagaccctagaaactt

SEQ ID NO: 208 - DNA, 113 bp, Helix aperta
  source 1..113: mol_type = genomic DNA
gtctggtatgaacgaacatttgggtgacaactgtctaaattaatttttctaaacttagttagcaagtgcaaatacttgtatgtatataaaagacgagaagaccctagaaactt

SEQ ID NO: 209 - DNA, 155 bp, Helix albescens
  source 1..155: mol_type = genomic DNA
tgactgtgcaaaggtagcataatcagttgactcttaattagggtctggtatgaaagaaaatatgggtagtaactgtcttaatatatttatttgaaattaataagcaagtgcaaatacttgcatacataaaaaagacgagaagaccctagaaactt

SEQ ID NO: 210 - DNA, 157 bp, Tyrrhenaria ceratina
  source 1..157: mol_type = genomic DNA
tgactgtgcaaaggtagcataatcagttggcttttaattagagtctggtatgaatgaaaatatgggtggtaactgtctctgacatattaatttgaactttttttagcaagtgcaaatacttgcgagggtaaaaaagacgagaagaccctagaaactt

SEQ ID NO: 211 - DNA, 154 bp, Helix vladika
  source 1..154: mol_type = genomic DNA
tgactgtgcaaaggtagcataatcagttggcttttaattggagtctggcatgaatgaaaatatgggtagtaactgtcttagcatattatttgaacttattaaacaagtgcaaatgcttgtaattacacaatagacgagaagaccctagaaactt

SEQ ID NO: 212 - DNA, 156 bp, Pleurodonte discolor
  source 1..156: mol_type = genomic DNA
tgactgtgcaaaggtagcataatcagttggcttataattgaagtcttgtatgaaggaataaatgggttacaactgtctctacagataaatagcgaatttacttaataagtgaaaatacttatagttttttaaaagacgagaagaccctagaaattt

SEQ ID NO: 213 - DNA, 154 bp, Pleurodonte lychnuchus
  source 1..154: mol_type = genomic DNA
tgactgtgcaaaggtagcataatcagttggcttataattgaagtctagtatgaatgaaggaatgggtgagtcctgtctccagaaaagaggtgaacttacctaacaagtgaaaatgcttgtacaaagacaaaagacgagaagaccctagaaattt

SEQ ID NO: 214 - DNA, 121 bp, Erctella mazzullii
  source 1..121: mol_type = genomic DNA
catattgaggtctggtatgatgaaattttgggtagaaactgtcttaatatattttttataatttaatatgcaagtgaaaatacttgctttaatataaaagacgagaagaccctagaaactt

SEQ ID NO: 215 - DNA, 115 bp, Erctella cephalaeditana
  source 1..115: mol_type = genomic DNA
gaagtctggtatgaatgaactttgggtagtaactgtcttaatatattttaaaaaatttaatatgtaagtgaaaatacttactttaatataaaagacgagaagaccctagaaactt

SEQ ID NO: 216 - DNA, 154 bp, Pleurodonte formosa
  source 1..154: mol_type = genomic DNA
tgactgtgcaaaggtagcataatcagttggcttataattgaagtctggtatgaaggaataaatgggtagtttctgtctttaggggaacgacgaacttactcagcaagtgaaaatacttgtatattaacaaaagacgagaagaccctagaaattt

SEQ ID NO: 217 - DNA, 156 bp, Helix christophi
  source 1..156: mol_type = genomic DNA
tgactgtgcaaaggtagcataatcagttgactcttaattggggcctgggatgaatgaaataatgggcggcagctttctcagatatatttctataaacttggttagtaggtgcaaatacctctatgtagataaaagacgagaagaccctagaagctt

SEQ ID NO: 218 - DNA, 156 bp, Helix nordmanni
  source 1..156: mol_type = genomic DNA
tgactgtgcaaaggtagcataatcagttggcttttaattggagtctgggatgaatgaaataatgggcggcaactttctcaaatatattattatgaatttggttagtaggtgcaaatacctacataaaaacaaaagacgagaagaccctagaagctt

SEQ ID NO: 219 - DNA, 155 bp, Pleurodonte nucleola
  source 1..155: mol_type = genomic DNA
tgactgtgcaaaggtagcataatcagttgtcttttaattgaagtctggcatgaatgaataaatgggtaggaactgtctcaagaaagaaacaaaaatttacttagtaagtgaaaatacttatgttttgacaaaagacgagaagaccctagaaattt

SEQ ID NO: 220 - DNA, 156 bp, Pleurodonte parilis
  source 1..156: mol_type = genomic DNA
tgactgtgcaaaggtagcataatcagttggcttttaattgaagtctagtatgaaggaacaaatgggtgaaagctgtctttagtagaaaacaaggaacttacttagtaagtgaaaatacttacagttagataatagacgagaagaccctagaaattt

SEQ ID NO: 221 - DNA, 156 bp, Gonostomopsis auridens
  source 1..156: mol_type = genomic DNA
tgactgtgcaaaggtagcataatcagttggcttataattgaagtctagtatgaaagaataaaatgggcgggcgctgtcttcttaaggtatctagaacttacttagtaagtgaaaatacttacatataaataaaagacgagaagacccttgaagttt

SEQ ID NO: 222 - DNA, 157 bp, Caracolus caracollus
  source 1..157: mol_type = genomic DNA
tgactgtgcaaaggtagcataataatttggcttttaattggagtctggaatgaatgggtacatggatataggctgtcttatactaattacttataacttttttaattaagtgaaaatacttaaagaaatataatagacgagaagaccctagaaattt

SEQ ID NO: 223 - DNA, 158 bp, Lacteoluna selenina
  source 1..158: mol_type = genomic DNA
tgactgtgcaaaggtagcataataaattggcttataattgaagtcgggaatgaatggattaatgaatataaactgtcttatctaaattttaattaattttcttagtaagtgaaaattcttacaatgtgttttattagacgagaagacccttgaaattt

SEQ ID NO: 224 - DNA, 156 bp, Cernuella cisalpine
  source 1..156: mol_type = genomic DNA
tgactgtgctaaggtagcataatcatttggcttataattgaagtctggcatgaaagaatttatgggtaacaactgtctcctgtaattatagatgaacttactcaataagtgaaaatgcttatataatcacaatagacgagaagaccctagaaattt

SEQ ID NO: 225 - DNA, 155 bp, Cochlicella acuta
  source 1..155: mol_type = genomic DNA
tgactgtgcaaaggtagcataatcaattggcttgtaatttaagtctggtatgaatgtagttttaggctgaaactgtctctttatatacattgaaatttacttagccggtgaaaatgccggcagttaaataatagacgagaagaccctagaaattt

SEQ ID NO: 226 - DNA, 156 bp, Disculella maderensis
  source 1..156: mol_type = genomic DNA
tgactgtgcaaaggtagcataatcatttggcttataattgaagtctagtatgaatgaattaatgggtaaaagctgtctctttataattaagtagaaattacttagagggtgaaaattctctcaatatcttaatagacgagaagaccctagaaattt

SEQ ID NO: 227 - DNA, 155 bp, Dialeuca nemoraloides
  source 1..155: mol_type = genomic DNA
tgactgtgcaaaggtagcataataaattggcttataattgaagtcttgaatgaaaggtaacacgagtaaaatctgtcttttagaaatatttttaatttacttattaagtgaaaattcttaatttaaaaaattagacgagaagaccctagaaattt

SEQ ID NO: 228 - DNA, 154 bp, Monadenia fidelis
  source 1..154: mol_type = genomic DNA
tgactgtgcaaaggtagcataatcagttggcctttaattggggtctggaatgaacgaagatttgggtaatagctgtctttatatggtaagcaaaacttgcttagtaagtgaaaattcttacatgataagaaagacgagaagaccctagaaattt

SEQ ID NO: 229 - DNA, 157 bp, Cepaea nemoralis
  source 1..157: mol_type = genomic DNA
tgactgtgcaaaggtagcataataatttggcttataattgaagtctggctcgaaagaattaatgggaagcagctgtctccaggggtactagtacaaaattagtaagtaagtgaaaatacttgcgggaagaaaatagacgagaagaccctagaagctt

SEQ ID NO: 230 - DNA, 155 bp, Sphincterochila candidissima
  source 1..155: mol_type = genomic DNA
tgactgtgctaaggtagcataatcaattgactcataattggagtctagcatgaatgaaagaatgggtaaacactgtctctgtcttaccgattgaaattactaagcaggtgaaaatacctgtattcaaataatagacgagaagaccctagaagacc

SEQ ID NO: 231 - DNA, 156 bp, Microphysula ingersolli
  source 1..156: mol_type = genomic DNA
taactgtgcaaaggtagcataataatttggcttataattgaagtctagaatgaaaggaagcatgggcgaattctgtttcttaattttttttttgaaattaataataaggtgaaaattccttttaaattctattagacgagaagaccctacagagct

SEQ ID NO: 232 - DNA, 155 bp, Helicodonta obvoluta
  source 1..155: mol_type = genomic DNA
tgactgtgcaaaggtagcataataatttggcttataattgaagtctggtatgaatgaatatatgggtaccaactgtctccactatatagcatgaatttactgattgggtgcaaattcccatcgcaagataatagacgagaagaccctagaaattt

SEQ ID NO: 233 - DNA, 155 bp, Cernuella virgata
  source 1..155: mol_type = genomic DNA
tgactgtgctaaggtagcataatcatttggcttataattgaagtctggcatgaaggaatttatgggtaacaactgtctcctataactgtaatgaacttgctaaataagtgaaaatacttatgtaaccataatagacgagaagaccctagaaattt

SEQ ID NO: 234 - DNA, 227 bp, Loligo forbesii
  source 1..227: mol_type = genomic DNA
gggacgagaagaccctattgagcttataattttattattattattatttgtaattggtgtaataatatataaattttaattggggtgattaaggaataaaaattaattaatatgattaaataacttccttagaaattatattgttaaataaagtaacctatarttattataaaaatttataatcagtattgcttataatatagttaccatagggataacagcgtaat

SEQ ID NO: 235 - DNA, 196 bp, Nototodarus sloanii
  source 1..196: mol_type = genomic DNA
gggacgagaagaccctaatgagcttataattttattgttatggtttaatatatttaaatgataatatataaattttaattggggtgattaaggaataaattatgattaataacttccttaggttaaatatatattgattaagtaaccaataaatatattgcttataatatagttaccatagggataacagcgtaat

SEQ ID NO: 236 - DNA, 197 bp, Sepia lorigera
  source 1..197: mol_type = genomic DNA
gggacgagaagaccctattgagctttaaatttgaagtattgtatttattaatataatatttaaaaattttaattggggtgattaaggaataaaaattataatatattaaaaatttaacttccttaaataatatattgttaaataaagtaaccaataatattgcttatataaagttaccatagggataacagcgtaat

SEQ ID NO: 237 - DNA, 197 bp, Sepia pardex
  source 1..197: mol_type = genomic DNA
gggacgagaagaccctattgagctttaaatttggaatattatatttgttagtatagtatttaaaaaattttaattggggtgattaaggaataaaattataagtatattaaaatttaacttccttaaataatatattattaaataaagtaaccaataatattgcttatataaagttaccatagggataacagcgtaat

SEQ ID NO: 238 - DNA, 198 bp, Sepia kobiensis
  source 1..198: mol_type = genomic DNA
gggacgagaagaccctattgagctttaaatttgggaatattatatttattaagtataatgtttgaaaaattttaattggggtgattaaggaataaaataaaatatattaaaaatttaacttccttagataatatattgttaaataaagtaaccaataatattgcttatataaagttaccatagggataacagcgtaat

SEQ ID NO: 239 - DNA, 194 bp, Rossia pacifica
  source 1..194: mol_type = genomic DNA
ggacgagaaagaccctattgagcttgtaattttattttattgtttattaaggatagtaaagtataaattttaattggggtgattaaggaataagataatataagataacttccttaaaggttagtataggtgtatgaagtaaccaataaattatattgcttatattatagttaccatagggataacagcgtaat

SEQ ID NO: 240 - DNA, 194 bp, Berryteuthis magister
  source 1..194: mol_type = genomic DNA
gggacgagaagaccctattgagcttataattttattgctactaaattattattagtgtagtagtatataaattttaattggggtgattaaggaataatttagtttagtaacttccttaaatttacatattggtggataaaagtaacccataaagtattgcttataatatagtaccatagggataacagccgaat

SEQ ID NO: 241 - DNA, 204 bp, Eledone massyae
  source 1..204: mol_type = genomic DNA
ggaacgagaagaccctattgagcttaatattaaaatataaattttgttagtaataaattaataaagtttattaattttaaattttgattggggtgatcgaggaatataaataattaaatttaacttcctaatattatatatttataaataaagaaaccaaatattttgcttagaaggaagttaccatagggattacagcgtgat

SEQ ID NO: 242 - DNA, 185 bp, Sepia robsoni
  source 1..185: mol_type = genomic DNA
gggacgagaagaccctattgagcttaaatttaattacataattaaattatgttttgataaattttaattggggtgattaaggaataaaaatattatatagtaacttccttaatagttaatactgttgggttaattaaccaataatattgcttataataaagttaccatagggataacagcgtaat

SEQ ID NO: 243 - DNA, 222 bp, Loligo reynaudii
  source 1..222: mol_type = genomic DNA
gggacgagaagaccctactgagcttgtaattttattattactgctatttgtaattggggcagtaatatataaattttaattggggtgattaaggaataaaataaatatatcttagtaacttccttataaagtatattgttagatgaagtaaccaattaaagttacatgatattgtaataaatattgcttataatatagttaccatagggatcacagcgtaat

SEQ ID NO: 244 - DNA, 212 bp, Doryteuthis (Amerigo) pealeii
  source 1..212: mol_type = genomic DNA
gggacgagaagaccctattgagcttataattttattattgtcagtattgttaatttttattggcactatataaattttaattggggtgattaaggaataaggtttatatatttcagtaacttccttagtagtagataaattgttagataaagtaaccaatattattagatttattgcttatactatagttaccatagggataacagcgtaat

SEQ ID NO: 245 - DNA, 213 bp, Doryteuthis (Amerigo) gahi
  source 1..213: mol_type = genomic DNA
gggacgagaagaccctattgagcttataattttattattaccaatattattaattgttagtggtaatatataaattttaattggggtgattaaggacataaaaagtttattatttagtaacttccttagtaaatataaattgttaaataaagtaaccaatataaataaatattattgcttatattatagttaccatagggataacagcgtaat

SEQ ID NO: 246 - DNA, 193 bp, Sepiola rondeletii
  source 1..193: mol_type = genomic DNA
gggacgagaagaccctattgagcttataattttattattattattatatgtaataataaatgaattttaattggggtgattaaggaataaattagtattagttagtaacttccttaaatagataaatagatgtataaattaaccaataattatattgcttttattaaagttaccatagggataacagcgtaat

SEQ ID NO: 247 - DNA, 191 bp, Sepiola robusta
  source 1..191: mol_type = genomic DNA
gggacgagaagaccctattgagcttgtaattttattagtattattaatataatatttaataaattttaattggggtgattaaggaataaattaatattattagtaacttccttaagtaataggataggtgtataaattaaccaataattgtattgcttttatcaaagttaccatagggataacagcgtaat

SEQ ID NO: 248 - DNA, 191 bp, Sepiola intermedia
  source 1..191: mol_type = genomic DNA
gggacgagaagaccctattgagcttataattttattagtattattaatataatatttaataaattttaattggggtgattaaggaataaattaatattattagtaacttccttaagtaataggataggtgtataaattaaccaataattgtattgcttttatcaaagttaccatagggataacagcgtaat

SEQ ID NO: 249 - DNA, 193 bp, Adinaefiola ligulata
  source 1..193: mol_type = genomic DNA
gggacgagaagaccctattgagcttttaattttgatatattatatttatataatgttgaataaattttaattggggtgattaaggaataaatataattaattagtatcttccttaaaataatagtatagatatataaattaaccaataattatattgcttatattagagttaccatagggataacagcgtaat

SEQ ID NO: 250 - DNA, 192 bp, Sepia smithi
  source 1..192: mol_type = genomic DNA
gggacgagaagaccctattgagcttaaatttaattttattaattatgatattagtaaataataaattttaattggggtgattaaggaataaatataataataagtaataacttccttaaattattaaactgttaggtgaagtaaccaataatattgcttatattaaagttaccatagggataacagcgtaat

SEQ ID NO: 251 - DNA, 193 bp, Sepia elliptica
  misc_feature 4: note = n is a, c, g, or t
  misc_feature 34: note = n is a, c, g, or t
  source 1..193: mol_type = genomic DNA
gggncgagaagaccctattgagcttaaattaatngtatattaaattaagattaatatattaataattttaattggggtgattaaggaataaaaaatattgtaaaatgataacttccttaaatttaatactgttaggtagagtaaccaatatttattgcttatattaaagttaccatagggataacagcgtaat

SEQ ID NO: 252 - DNA, 186 bp, Eledone palari
  source 1..186: mol_type = genomic DNA
agggacaaaagaccctattgagcttttattattttttaaattattattaataagaaatttatgcaaataatttgattggggtgatcaaggaataaagaaataacttccttaaatattaaggtttaataggaaaataaaccaaatttttgcttttatgatgagttaccatagggataacagggtaat

SEQ ID NO: 253 - DNA, 221 bp, Eledone moschata
  source 1..221: mol_type = genomic DNA
gggacgagaagaccctattgagcttaatgttaaattataaataatttaatgatcttattatgttttacattatttataaaatttaaattttgattggggtgatcaaggaataaaagtagtgtagtgtttataacttccttataggatttatgagttattaatggtataataaaccaaatattttgcttataagtaagttaccatagggataacagcgtaat

SEQ ID NO: 254 - DNA, 191 bp, Sepiola affinis; identical to SEQ ID NO: 248
  source 1..191: mol_type = genomic DNA

SEQ ID NO: 255 - DNA, 191 bp, Rossia palpebrosa
  source 1..191: mol_type = genomic DNA
gggacgagaagaccctactgagcttgtaattttattttattatttttaggtagtagggtataaattttaattggggtgattaaggaataaagtgtattagggtaacttccttaagtgatagtatatgtgtattaagtaaccaataaattatattgcttatattatagttaccatagggataacagcgtaat

SEQ ID NO: 256 - DNA, 195 bp, Gonatus madokai
  source 1..195: mol_type = genomic DNA
gggacgagaagaccctattgagcttataattttatcattattaggtttattattaatatagtgatgtataaattttaattggggtgattaaggaataaatttaatttagtaacttccttaagttttatattgttgaataaagtaaccaataaattattgcttataatatagttaccatagggataacagcgtaat

SEQ ID NO: 257 - DNA, 195 bp, Gonatus kamtschaticus
  source 1..195: mol_type = genomic DNA
gggacgagaagaccctattgagcttataattttatcattattagatttattattaatatagtgatgtataaattttaattggggtgattaaggaataaatttaatttagtaacttccttaagttctatattgttgaataaagtaaccaataaattattgcttataatatagttaccatagggataacagcgtaat

SEQ ID NO: 258 - DNA, 214 bp, Eledone cirrhosa
  source 1..214: mol_type = genomic DNA
gggacgagaagaccctattgagcttgatattaaaatataaattttttttaaagttaatataattatgttatgggtttaaattttgattggggtgatcaaggaataaaaaagattaatgtatatatatattaacttccttatatataaatatttatagataaataaaccgagagttttgcttaaaagtaagttaccatagggataacagcgtaat

SEQ ID NO: 259 - DNA, 181 bp, Sepia elegans
  source 1..181: mol_type = genomic DNA
gggaagagaagaccctattgagctttaattttaataggttataataaatattatagtaaaatagaaattttaattggggtgattaaggaataaaaaagtaacttccttaaaaataaaattgttaggttaagtaaccaattgtattgcttatataaagttaccatagggataacagcgtaat

SEQ ID NO: 260 - DNA, 189 bp, Rossia bipillata
  source 1..189: mol_type = genomic DNA
gggacgagaagaccctagtgagcttataattttattttgttgttagtaaatagtagagatgaaattttaattggggtgattaaggaataaaatatgtttatataacttccttagatttggtataggtatataaagtaaccaataaatatattgcttatattatagttaccatagggataacagcgtaat

SEQ ID NO: 261 - DNA, 189 bp, Sepiola atlantica
  source 1..189: mol_type = genomic DNA
gggacgagaagaccctattgagcttataattttattattattatatgtaataataaatgaattttaattggggtgattaaggaataattagtattagttagtaacttccttaaatagataaatagatgtataaattaaccaataattatattgcttttattaaagttaccatagggataacagcgtaat

SEQ ID NO: 262 - DNA, 206 bp, Lolliguncula (Lolliguncula) panamensis
  source 1..206: mol_type = genomic DNA
gggacgagaagaccctattgagcttgtaattttattgttattattatattaattgttaatggtaatatataaattttaattggggtgattaaggaataaaattttatattagtaacttccttaataagatgattgttagataaagtaaccaatattattaaatattattgcttatattgtagttaccatagggataacagcgtaat

SEQ ID NO: 263 - DNA, 193 bp, Octopus maya
  source 1..193: mol_type = genomic DNA
gggacgaaaagaccctattgagcttatattattttgtaaattatatgattgttgtttattggaattaattttgattggggtgatcaaggaataaattattattaaaattaacttccttaattaattattttatggagaaataaaccaaattttttgcttagatgataagttaccatagggataacagcgtaat

SEQ ID NO: 264 - DNA, 213 bp, Illex illecebrosus
  source 1..213: mol_type = genomic DNA
gggacgagaagaccctactgagcttataattttattatcacatatatttatgtataatgtgctatataaattttaattggggtgattaaggaataatatagctattttataacttccttagatattatattgttgaattaagtaaccgattaattaatagaattgtattatgtattgcttataatatagttaccatagggataacagcgtaat

SEQ ID NO: 265 - DNA, 196 bp, Nototodarus gouldi
  source 1..196: mol_type = genomic DNA
gggacgagaagaccctaatgagcttataattttattgttgtggtttaatatatttaaatgataatatataaattttaattggggtgattaaggaataaattatgattaataacttccttaggctgaatatatattgattaagtaaccaataaatatattgcttataatatagttaccatagggataacagcgtaat

SEQ ID NO: 266 - DNA, 193 bp, Gonatopsis octopedatus
  source 1..193: mol_type = genomic DNA
gggacgagaagaccctattgagcttataattttatcattattagatttattattagtgtagtgtgtataaattttaattggggtgattaaggaataatttaatttattaacttccttaagttacatattgttgaataaagtaaccaataaattattgcttataatatagttaccatagggataacagcgtaat

SEQ ID NO: 267 - DNA, 195 bp, Illex coindetii
  source 1..195: mol_type = genomic DNA
gggacgagaagaccctaatgagcttataattttattattattatttaatatatttaaataataatatataaattttaattggggtgattaaggaataaagaattatataataacttccttagattaaatatatatagataaagtaaccaataatatattgcttataatatagttaccatagggataacagcgtaa

SEQ ID NO: 268 - DNA, 195 bp, Berryteuthis anonychus
  source 1..195: mol_type = genomic DNA
gggacgagaagaccctaatgagcttataattttattgttgttaaatttattattagtaaagcagcatataaattttaattggggtgattaaggaataaattaatttagtaacttccttatatttaaatattgttaaataaagtaaccaataaattattgattataatatagttaccatagggataacagcgtaat

SEQ ID NO: 269 - DNA, 196 bp, Gonatus fabricii
  source 1..196: mol_type = genomic DNA
gggacgagaagaccctattgagcttataattttatcattattagatttattattagtgtaatgttgtataaattttaattggggtgattaaggaataatattaatttagtaacttccttaagttatatattgttgaataaagtaaccaattaaattattgcttataatatagttaccatagggataacagcgtaat

SEQ ID NO: 270 - DNA, 193 bp, Lusepiola birostrata
  source 1..193: mol_type = genomic DNA
gggacgagaagaccctattgagcttgtaattttattttattgtttattaggatagtaaagtataaattttaattggggtgattaaggaataagataatataagataacttccttaaaggttagtataggtgtatgaagtaaccaataaattatattgcttatattatagttaccatagggataacagcgtaat

SEQ ID NO: 271 - DNA, 206 bp, Octopus tetricus
  misc_feature 10: note = n is a, c, g, or t
  source 1..206: mol_type = genomic DNA
ggacgaaaangaccctattgagcttttgttattatataaatatacatataaatgtataatttatttataaataactttggttggggtgatcaaggaataaaaataatttaaaatgttgtgttaacttccttaattttgagtttaattgaagaataaaccaagttttttgcttagaagataagttaccatagggataacagcgtaat

SEQ ID NO: 272 - DNA, 222 bp, Uroteuthis (Photololigo) sibogae
  source 1..222: mol_type = genomic DNA
gggacgagaagaccctattgagcttgtaattctgttattattatgtttattaaatatcattaatagtatatgaattttaattggggtgattaaggaataaataatttataatatataacttccttagagtattatattgttgaatgaattaaccgatataattaacacaggttgtgttgtgtattgcttataatatagttaccatagggataacagcgtaat

SEQ ID NO: 273 - DNA, 212 bp, Doryteuthis (Doryteuthis) pleii
  source 1..212: mol_type = genomic DNA
gggacgagaagaccctattgagcttataattttattgttgtcagtgagtattaatttcactgttggtatataaattttaattggggtgattaaggaataaagtttatatattaataacttccttagagttaatattattgttagataaagtaaccaataaattgttaagattattgcttatactatagttaccatagggataacagcgtaat

SEQ ID NO: 274 - DNA, 207 bp, Doryteuthis sanpaulensis
  source 1..207: mol_type = genomic DNA
gggacgagaagaccctattgagcttataattttattgtcatcaagattgttatgtaactggtgacatataaattttaattggggtgattgaggaataaagttttattgtggaagtaacttccttagtaatgcatattgttaaatgaagtaaccaataattaattactattgcttatactatagttaccatagggataacagcgtaat

SEQ ID NO: 275 - DNA, 218 bp, Doryteuthis (Amerigo) surinamensis
  source 1..218: mol_type = genomic DNA
gggacgagaagaccctattgagcttataattttattaccgttagtagtgattaaaattttactaagggtatataaattttaattggggtgattaaggaataaaagcttatgtattaataacttccttagaaataaataaaaaattgttagataaagtaaccaataaattgttaaaattattgcttatactatagttaccatagggataacagcgtaat

SEQ ID NO: 276 - DNA, 192 bp, Octopus hubbsorum
  source 1..192: mol_type = genomic DNA
gggacgaaaagaccctattgagcttgtattatttaacaaattatatgattgttatttgtttgaattaattttgattggggtgatcaaggaataaattttatttagtttaacttccttaattaattgttttatagaagaataaaccaaagtttttgcttagaagataagttaccatagggataacagcgtaat

SEQ ID NO: 277 - DNA, 239 bp, Macrotritopus defilippi
  misc_feature 46..48: note = n is a, c, g, or t
  misc_feature 61..66: note = n is a, c, g, or t
  misc_feature 84..85: note = n is a, c, g, or t
  misc_feature 139..150: note = n is a, c, g, or t
  misc_feature 168..170: note = n is a, c, g, or t
  source 1..239: mol_type = genomic DNA
gggacgaaaagaccctattgagcttgtgttaataaataaatactannntatatatatatannnnnntatatatatatatattanntttgtgagttttaaatttggttggggtgatcaaggaataaaattaaaataaaannnnnnnnnnnngtaacttccttaaaaatnnnaatattaatgggttaataaaccaagttttttgcttataaggtaagttaccatagggataacagcgtaat

SEQ ID NO: 278 - DNA, 192 bp, Octopus insularis
  source 1..192: mol_type = genomic DNA
gggacgaaaagaccctattgagcttatattattttataaattatatgattgttgtttatttagaataattttgattggggtgatcaaggaataagattgtttaaattaacttccttaattaatttgttttgtggaaaaataaaccaaattttttgcttagaagataagttaccatagggataacagcgtaat

SEQ ID NO: 279 - DNA, 221 bp, Loliolus (Nipponololigo) sumatrensis
  source 1..221: mol_type = genomic DNA
gggacgagaagaccctattgagcttataattttattactattatgtttgttaaattcatcattgtagtatataaattttaattggggtgattaaggaataagttttttataatgtaacttccttagattaatatattgttagattaagtaaccgatgtgattaacataatatatgttatgtattgcttataatatagttaccatagggataacagcgtaat

SEQ ID NO: 280 - DNA, 199 bp, Sepia stellifera
  source 1..199: mol_type = genomic DNA
gggacgagaagaccctattgagcttaaatttaaaaatattaaataatttaatattttgataaattttaattggggtgattaaggaataaaataataggtattattaaatatagtaacttccttaaatttaatactgttaggttaagtaaccaatgtaatattgcttataataaagttaccatagggataacagcgtaat

SEQ ID NO: 281 - DNA, 193 bp, Sepia recurvirostra
  source 1..193: mol_type = genomic DNA
gggacgagaagaccctattgagcttgaatttaattatattattttaataatattttgataaattttaattggggtgattaaggaataaaaatttatatatattatgtagtaacttccttaattattatactgttaggttaagtaaccaataatattgcttataataaagttaccatagggataacagcgtaat

SEQ ID NO: 282 - DNA, 192 bp, Sepia madokai
  source 1..192: mol_type = genomic DNA
gggacgagaagaccctattgagctttaatttaattatataatatagtttgtattttgttaaattttaattggggtgattaaggaataagattagtattaattagtaacttccttaaattttattgttactgttgggtgaagtaaccaatattattgcttataataaagttaccatagggataacagcgtaat

SEQ ID NO: 283 - DNA, 199 bp, Sepia kobiensis; identical to SEQ ID NO: 280
  source 1..199: mol_type = genomic DNA

SEQ ID NO: 284 - DNA, 191 bp, Amphioctopus aegina
  source 1..191: mol_type = genomic DNA
gggacgaaaagaccctattgagctttatattatactttaaaattatttattgtttttatttaaatataattttggttggggtgatcaaggaataaataattaattttaacttccttaatttgtttatagttatatgaataaaccaaattttttgcttagaagataagttaccatagggataacagcgtaat

SEQ ID NO: 285 - DNA, 191 bp, Sepia officinalis
  source 1..191: mol_type = genomic DNA
gggacgagaagaccctaatgagcttaaattttattttattatatatattttatagtaaaaaagaagttttagttggggtgattaaggaataaaaaaacgaatgatgtaacttccttaaatataaaattgttaaattaagtaaccaatttatattgcttataatagagttaccatagggataacagcgtaat

SEQ ID NO: 286 - DNA, 191 bp, Sepioteuthis lessoniana
  source 1..191: mol_type = genomic DNA
gggacgagaagaccctattgagcttgtagtttttatataactatattattaatttatatagttatttataaactttagttggggtgactaaggaataatataagtaacttccttagagtaaagattgttaaataaagtaaccaaatttaatattgcttataatatagttaccatagggataacagcgtaat

SEQ ID NO: 287 - DNA, 196 bp, Todarodes pacificus
  source 1..196: mol_type = genomic DNA
gggacgagaagaccctaatgagcttataattttattattgtgatttaatatatttaaatgataatatataaattttaattggggtgattaaggaataatttataagtagtaacttccttagattaaatatatattgattaagtaaccaataaatatattgcttataatatagttaccatagggataacagcgtaat

SEQ ID NO: 288 - DNA, 211 bp, Octopus vulgaris
  source 1..211: mol_type = genomic DNA
gggacgaaaagaccctattgagcttttgttattatatagatatatatatgtatatatatatgatttatttgtaaataactttggttggggtgatcaaggaataaaaattaatttaaagtgttttattaacttccttaattttaaatttagttgaagaataaaccaagttttttgcttagaagataagttaccatagggataacagcgtaat

SEQ ID NO: 289 - DNA, 187 bp, Heterololigo bleekeri
  source 1..187: mol_type = genomic DNA
ggacgagaagaccctatatttaattcttattatttttttagatttattttaattggggtgattaaggaataaagtatattagttagtaacttccttagtaggatatattgttgaataaagtaaccgatattacgtatagaaccgtatattgcttataatatagttaccatagggataacagcgtaat

SEQ ID NO: 290 - DNA, 211 bp, Octopus sinensis
  source 1..211: mol_type = genomic DNA
gggacgaaaagaccctattgagcttttgttattatatagatatatatatatatatatatatgatttatttgtaaataactttggttggggtgatcaaggaataaaaattaatttaaagtgttttattaacttccttaattttaaatttagttgaagaataaaccaagttttttgcttagaagataagttaccatagggataacagcgtaat

SEQ ID NO: 291 - DNA, 204 bp, Octopus americanus
  source 1..204: mol_type = genomic DNA
gggacgaaaagaccctattgagcttttgttgttatatagatatatataaatatataatttatttgtaaataactttggttggggtgatcaaggaataaaattaatttaaagtgttatattaacttccttaattttgaatttagttgaagaataaaccaagttttttgcttagaagataagttaccatagggataacagcgtaat

SEQ ID NO: 292 - DNA, 196 bp, Narrowteuthis nesisi
  source 1..196: mol_type = genomic DNA
gggacgagaagaccctattgagcttataattttactgttgtttataagtatttatttgatgatggataaattttaattggggtgattgaggaataaaatatattaaatagtaacttccttaggttaaatgttgttggataaagtaaccaatatattattgcttataatatagttaccatagggataacagcgtaat

SEQ ID NO: 293 - DNA, 195 bp, Ommastrephes bartramii
  source 1..195: mol_type = genomic DNA
gggacgagaagaccctaatgagcttataattttattatcattatttaaaacatttaggtggtggtatataaattttaattggggtgattaaggaataaattgtgtatagtaacttccttaaattaggtatttgtagataaagtaaccaatgtaatattgcttatagtatagttaccatagggataacagcgtaat

SEQ ID NO: 294 - DNA, 194 bp, Sepiella japonica
  source 1..194: mol_type = genomic DNA
gggacgagaagaccctaatgagcttaaattttatattattgttatatatgttttataattaattggaaattttaattggggtgattaaggaataatattaagataaataacttccttatataataataaattgttgaattaagtaaccaatattattgcttttaataaagttaccatagggataacagcgtaat

SEQ ID NO: 295 - DNA, 220 bp, Uroteuthis (Photololigo) edulis
  source 1..220: mol_type = genomic DNA
gggacgagaagaccctattgagcttataattttattaatattttataaatatttatgttgtgttatataaattttaattggggtgattaaggaataattttattgattttatagtgtaacttccttagaaaacatattgttaaattaagtaaccgatttatatataggtttatattgggtattgcttataatatagttaccatagggataacagcgtaat

SEQ ID NO: 296 - DNA, 200 bp, Doryteuthis (Amerigo) opalescens
  source 1..200: mol_type = genomic DNA
gggacgagaagaccctattgagcttgtaattttattattattagtattatttattcttattggtagtatataaattttaattggggtgattaaggaataaggtttgtagttttagtaacttccttattaataatgattgttaaataaagtaaccaatattattgcttatactatagttaccatagggataacagcgtaat

SEQ ID NO: 297 - DNA, 195 bp, Architeuthis dux
  source 1..195: mol_type = genomic DNA
gggacgagaagaccctattgagcttataattttattgttgttttgtaattatttatgtgatgatagataaattttaattggggtgattgaggaataaggtaatagtagtaacttccttagaaataaatgttgttgggtgaagtaaccaatatattattgcttataatatagttaccatagggataacagcgtaat

SEQ ID NO: 298 - DNA, 195 bp, Dosidicus gigas
  source 1..195: mol_type = genomic DNA
gggacgagaagaccctaatgagcttgtaattttattattgttatttaaaatatttagatagcaatatataaattttaattggggtgattaaggaataagttgaatatagtaacttccttaaattgaatatttatagataaagtaaccaatataatattgcttataatatagttaccatagggataacagcgtaat

SEQ ID NO: 299 - DNA, 194 bp, Sepia esculenta
  source 1..194: mol_type = genomic DNA
gggacgagaagaccctattgagcttaaatttaaaagtactatagtaatttttttaatattttgataaattttaattggggtgattaaggaataaaaggataataaattggtaacttccttaaacttaatactattaggttaagtaaccaataatattgcttataataaagttaccatagggataacagcgtaat

SEQ ID NO: 300 - DNA, 196 bp, Amphioctopus fangsiao
  source 1..196: mol_type = genomic DNA
gggacgaaaagaccctattgagctttatattattgtttaaatttattatgctgtttttatttaaaataattttggttggggtgatcaaggaataaaatattaaatttattaacttccttattatatttaaatatctatattaataaaccaaactttttgcttagaagataagttaccatagggataacagcgtaat

SEQ ID NO: 301 - DNA, 219 bp, Loligo vulgaris
  source 1..219: mol_type = genomic DNA
gggacgagaagaccctactgagcttataattttattattattgttatttttattggagcagtaatatataaattttaattggggtgattaaggaataaataaatatatttagtaacttccttataaagaatattgttagatgaagtaaccaataaagttacatgattattgtaataaatattgcttataatatagttaccatagggataacagsgtaat

SEQ ID NO: 302 - DNA, 192 bp, Octopus mimus
  source 1..192: mol_type = genomic DNA
gggacgaaaagaccctattgagcttgtattatttaacaaattatatgattgttatttgtttgaattaattttgattggggtgatcaaggaataaattttatttagtttaacttccttagttaattgttttatagaagaataaaccaaagtttttgcttagaagataagttaccatagggataacagcgtaat

SEQ ID NO: 303 - DNA, 230 bp, Octopus conispadiceus
  source 1..230: mol_type = genomic DNA
gggacgagaagaccctattgagctttatattagttataataaataataaaaatataatttatttggttaaattagtattatattaataatatttattatgtttaattttgattggggtgatcaaggaataaagtggatattatttaacttccttaaaatttaatttttttagaataattaaccaatgatattgcttatatgataagttaccatagggataacagcgtaat

SEQ ID NO: 304 - DNA, 195 bp, Octopus bimaculoides
  source 1..195: mol_type = genomic DNA
gggacgaaaagaccctattgagcttatattatttattaaattatatagttatagtttattataaattaattttgattggggtgatcaaggaataaattttattattatataacttccttagttaaaatagtttttggaaaaataaaccaagttttttgcttagaagataagttaccatagggataacagcgtaat

SEQ ID NO: 305 - DNA, 213 bp, Uroteuthis (Photololigo) chinensis; identical to SEQ ID NO: 264
  source 1..213: mol_type = genomic DNA

SEQ ID NO: 306 - DNA, 220 bp, Uroteuthis (Photololigo) duvaucelii
  source 1..220: mol_type = genomic DNA
gggacgagaagaccctattgagcttataattttattactattatggttattgaatttatcattgtagtatataaattttaattggggtgattaaggaataaatttttataatgtaacttccttagattattatattgttaaattaagtaaccgatgtgattagcacaatgtgtgttatgtattgcttataatatagttaccatagggataacagcgtaat

SEQ ID NO: 307 - DNA, 199 bp, Illex argentinus
  source 1..199: mol_type = genomic DNA
gggacgagaagaccctaatgagcttataattttattattattatttaatatatttaaataataatatataaattttaattggggtgattaaggaataaaagaattatataataacttccttagattaaatatatatatatataaagtaaccaataatatattgcttataatatagttaccatagggataacagcgtaat

SEQ ID NO: 308 - DNA, 194 bp, Sepia aculeata
  source 1..194: mol_type = genomic DNA
gggacgagaagaccctattgagcttgaatttaattgtattattttataatattttaataaattttaattggggtgattaaggaataaaaaattttatatattgtatagtaacttccttaatatgattgtactgttaggttaagtaaccaataatattgcttataatagagttaccatagggataacagcgtaat

SEQ ID NO: 309 - DNA, 193 bp, Sepiella inermis
  source 1..193: mol_type = genomic DNA
gggacgagaagaccctaatgagcttaaattttatattattattaatatataattataataattggaaattttaattggggtgattaaggaataatattaaaataaataacttccttatatattaataaattgttgaattaagtaaccaataatattgcttttaataaagttaccatagggataacagcgtaat

SEQ ID NO: 310 - DNA, 185 bp, Sepia lycidas
  source 1..185: mol_type = genomic DNA
gggacgagaagaccctattgagcttaaatttaattacataatttaattatgttttgataaattttaattggggtgattaaggaataaaattattatatagtaacttccttaatagttaatactgttgggttaaataaccaataatattgcatataataaagttaccatagggataacagcgtaat

SEQ ID NO: 311 - DNA, 197 bp, Sepia latimanus
  source 1..197: mol_type = genomic DNA
gggacgagaagaccctaatgagcttttaatttaatagtatttaatgtaaaattgaatgtaaagttgtataaattttaattggggtgattaaggaataaaattattttaaataataacttccttaaatgtagtattgttagattaattaaccaatattattgcttataatagagttaccatagggataacagcgtaat

SEQ ID NO: 312 - DNA, 192 bp, Sepia apama
  source 1..192: mol_type = genomic DNA
gggacgagaagaccctaatgagcttaaatttaatagtgctaaaattataagttaataggattatgaaaattttaattggggtgattaagggataatttattaaataataacttccttaaatataatattgttgaattaattaaccaatttatattgcttatagtatagttaccatagggataacagcgtaat

SEQ ID NO: 313 - DNA, 191 bp, Sepia pharaonis
  source 1..191: mol_type = genomic DNA
gggacgagaagaccctattgagcttaaatttagtaatattatgtttagtagtatttaataaattttaattggggtgattaaggaataaaattatatattgaataataacttccttaattgtgtaatactgttaggttaagtaaccaataatattgcttataataaagttaccatagggataacagcgtaat

SEQ ID NO: 314 - DNA, 216 bp, Loliolus (Nipponololigo) beka
  source 1..216: mol_type = genomic DNA
gggacgagaagaccctattgagcttataatttttgttagtgcttttattaatatttataattgtattatataaattttaattggggtgattaaggaataactgattggaaagtaacttccttagatgtgtgtattgttagattaagtaaccgattaattaatatattgtattagatattgcttataatatagttaccatagggataacagcgtaat

SEQ ID NO: 315 - DNA, 221 bp, Alloteuthis subulata
  source 1..221: mol_type = genomic DNA
gggacgagaagaccctattgagcttataattttattgttattatatttatattaatatagtaatttataaattttaattggggtgattaaggaataaagataaataattaaagtaacttccttaaaataaagtattgttaaataaagtaaccgataaattagtattaaaataatactatatattgcttatattatagttaccatagggataacagcgtaat

SEQ ID NO: 316 - DNA, 198 bp, Nototodarus hawaiiensis
  source 1..198: mol_type = genomic DNA
gggacgagaagaccctaatgagcttataattttattattatgatttgatatatttgaatgataatatataaattttaattggggtgattaaggaataaggttttatattaataacttccttaaattaaatatatatgtattaagtaaccaataaatatattgcttataatatagttaccatagggataacagcgtaat

SEQ ID NO: 317 - DNA, 189 bp, Sepia papuensis
  source 1..189: mol_type = genomic DNA
rggacgagaagaccctatgagcttaaatttattaatttttattagaatttataaaaatttataaaattttaattggggtgattaaggaataagtgtttaaatagtaacttccttagatataattttgttagattaattaaccaattaatattgcttataaaatagttaccgtagggataacagcgtaat

SEQ ID NO: 318 - DNA, 188 bp, Rossia macrosoma
  source 1..188: mol_type = genomic DNA
gggacgagaagaccctattgagcttataattttattttgttgtttattgaaatagtagggtataaattttaattggggtgattaaggaataaggtgatagataacttccttaggtaataatgtagatgtatgaagtaaccaataatgcattgcttatattatagttaccatagggataacagcgtaat

SEQ ID NO: 319 - DNA, 188 bp, Rossia macrosoma; identical to SEQ ID NO: 318
  source 1..188: mol_type = genomic DNA

SEQ ID NO: 320 - DNA, 218 bp, Lolliguncula (Lolliguncula) brevis
  source 1..218: mol_type = genomic DNA
gggacgagaagaccctattgagcttgtaattttattagttaccagttaatattatataaatatggtaacttataaattttaattggggtgattaaggaataaatgtttagttagtaacttccttagattaataaataaattgttaaatgaagtaaccaataaaatttaatgtataaatattgcttataatatagttaccatagggataacagcgtaat

SEQ ID NO: 321 - DNA, 221 bp, Lolliguncula (Loliolopsis) diomedeae
  source 1..221: mol_type = genomic DNA
gggacgagaagaccctattgagcttataattttattaattatcaatatttatatatgtattgatttggtaatttataaattttaattggggtgattaaggaataaattttatttatagtaacttccttagataagttaataaatgattgttaaatgaagtaaccaataaaaattaggtgatattgcttataatatagttaccatagggataacagcgtaat

SEQ ID NO: 322 - DNA, 215 bp, Afrololigo mercatoris
  source 1..215: mol_type = genomic DNA
gggacgagaagaccctattgagcttataattttgttattattatatttatattaatatagtaatttataaattttaattggggtgattaaggaataaagataaatatgtaagtaacttccttaataaattatattgttaaatgaagtaaccaatataatattagtaataatatatattgcttatagtatagttaccatagggataacagcgtaat

SEQ ID NO: 323 - DNA, 233 bp, Octopus minor
  source 1..233: mol_type = genomic DNA
gggacgagaagaccctattgagctttatttattaatatttaaatataaatatataatttatattatgaaaattatgttgtatatatatatatatttaattggatttaattttgattggggtgatcaaggaataaagataaagtaacttccttaaaattaatgattgtttttatagagtaataaaccaagttttttgcttataagataagttaccataggggtaacagcgtaat

SEQ ID NO: 324 - DNA, 197 bp, Octopus bimaculatus
  source 1..197: mol_type = genomic DNA
gggacgaaaagaccctattgagcttatattatttattaaattatagagttataatttattataaattaattttggttggggtgatcaaggaataagtttaatgattatataacttccttaactgaactatttttttggaaaaataaaccaagtttttttgcttagaagataagttaccatagggataacagcgtaat

SEQ ID NO: 325 - DNA, 233 bp, Octopus variabilis; identical to SEQ ID NO: 323
  source 1..233: mol_type = genomic DNA

SEQ ID NO: 326 - DNA, 211 bp, Octopus cyanea
  source 1..211: mol_type = genomic DNA
gggacgaaaagaccctattgagctttatatattaatatataaatttaattggtgatttataaaaagtttattaatattaattttggttggggtgatcaaggaataaaaggtaaattgtattttagataacttccttagaaaaataattgatggagtaataaaccaagatttttgcttataagataagttaccatagggataacagcgtaat

SEQ ID NO: 327 - DNA, 241 bp, Callistoctopus ornatus
  source 1..241: mol_type = genomic DNA
gggacgagaagaccctattgagcttggttattaataattaaatataaaataaattgtttttatggatttatttagtatgatagtatataaatatatttagttaaattaattttgattggggtgatcaaggaataaaatatttaaaaagtaacttccttaatttttattagagaattttaattgagtaataaaccaaagtttttgcttataagataagttaccataggggtaacagcgtaat

SEQ ID NO: 328 - DNA, 220 bp, Enteroctopus megalocyathus
  source 1..220: mol_type = genomic DNA
gggacgagaagaccctattgagctttatattagttataataaataataaaattttatttatttggttaaataatattatgtttattatgtttaattttgattggggtgatcaaggaataagataagtataatttaacttccttaaaaataatttttttatagaataattaaccaataatattgcttatatgataagttaccatagggataacagcgtaat

SEQ ID NO: 329 - DNA, 230 bp, Enteroctopus dofleini; identical to SEQ ID NO: 303
  source 1..230: mol_type = genomic DNA

SEQ ID NO: 330 - DNA, 204 bp, Sasakiopus salebrosus
  source 1..204: mol_type = genomic DNA
gggacgagaagaccctattgagctttatattagttataataaatggtaaaatataatttattacgtttattgtgtttaattttgattggggtgatcaaggaataaaataaatataatttaacttccttagaaaaattattttttagaataattaaccaatgatattgcttatatgataagttaccatagggataacagcgtaat

SEQ ID NO: 331 - DNA, 211 bp, Octopus berrima
  source 1..211: mol_type = genomic DNA
gggacgagaagaccctattgagcttaatattaatatttaaatataaaaatttttagtttttattttatatgaatgtttattgaatgtaattttgattggggtgatcaaggaataaaagtaaatattaacttccttaagttgttgattttattgagtaataaaccaaattttttgcttataagataagttaccataggggtaacagcgtaat

SEQ ID NO: 332 - DNA, 198 bp, Amphioctopus marginatus
  source 1..198: mol_type = genomic DNA
gggacgaaaagaccctattgagctttatattatattttaaatataatttgttttgtatttatttaaatataattttggttggggtgatcaaggaataaagtatatttgtattaacttccttagatattatatataaataaattaataaaccaaatttattgcttagaagataagttaccatagggataacagcgtaat

SEQ ID NO: 333 - DNA, 254 bp, Octopus maorum
  source 1..254: mol_type = genomic DNA
gggacgagaagaccctattgagcttgtatattaatatttaaatgttagatggtttatttattatattgttaataaatgaatagatttttgttgattaataagtatggatgtttaatttaattaattttgattggggtgatcaaggaataaaagaaaagaaatttaataacttccttaagttttgaaatgtttattgaataataaccaaatttttttgcttataagataagttaccataggggtaacagcgtaat

SEQ ID NO: 334 - DNA, 209 bp, Octopus fitchi
  source 1..209: mol_type = genomic DNA
gggacgagaagaccctattgagcttagaattattatttaattaatgtgataatttatttattagtttattgatatttatttatgtaattttgattggggtgatcaaggaataaaagtttataataacttccttaggattttgtaatttatagattaataaaccataattttgcttagaagataagttaccatagggataacagcgtaat

SEQ ID NO: 335 - DNA, 193 bp, Amphioctopus neglectus
  source 1..193: mol_type = genomic DNA
gggacgaaaagaccctattgagctttatattatattttaaatttgattttatatttatttacatataattttggttggggtgatcaaggaataaaatgtatttatattaacttccttaaatggtgatatatataaatgaataaaccaaattttttgcttagaagataagttaccatagggataacagcgtaat

SEQ ID NO: 336 - DNA, 215 bp, Loliolus (Nipponololigo) uyii
  source 1..215: mol_type = genomic DNA
gggacgagaagaccctattgagcttataattttattaatattttattgttatttataattatattatataaattttaattggggtgattgaggaataattaattaaatagtaacttccttaggtatttatattgttgaattaagtaaccgattaattaatatgttttatacaagtattgcttataatatagttaccatagggataacagcgtaat

SEQ ID NO: 337 - DNA, 216 bp, Loliolus (Nipponololigo) japonica
  source 1..216: mol_type = genomic DNA
gggacgagaagaccctattgagcttataattttattgatatttttattattatttataattatattatataaattttaattggggtgattaaggaataattagttaaaaaataacttccttaagtgtaatatattgttgaattaagtaaccgattaattaatatttttatattagtattgcttataatatagttaccatagggataacagcgtaat

SEQ ID NO: 338 - DNA, 194 bp, Bathyteuthis abyssicola
  source 1..194: mol_type = genomic DNA
gggacgagaagaccctattgagcttgtaattttattattattatattagtagtattgatatagtaatatataattttagttggggtgattaaggaataaattgtatagtaacttccttaggttatatattttattatgaagtaaccaatatagtattgcttatattatagttaccatagggataacagcgtaat

SEQ ID NO: 339 - DNA, 194 bp, Semirossia patagonica
  source 1..194: mol_type = genomic DNA
gggacgagaagaccctactgagcttgtaattttattttattatttattgtaatagtaatgtataaattttaattggggtgattaaggaataaatataatattaaagtaacttccttaaataaaaatatagatatataaagtaactaataattatattacttatattatagttaccatagggataacagcgtaat

SEQ ID NO: 340 - DNA, 251 bp, Cistopus taiwanicus
  source 1..251: mol_type = genomic DNA
gggacgaaaagaccctattgagcttatattaatatttatagtattataagtatatatatatatatatatatatatatatatatatatatatatatagagtatatgttataaatatattaattttggttggggtgatcaaggaataaagtatagaaaaatttttatataacttccttaatttttaattttattgattaataaaccaatatttttgcttatatgataagttaccatagggataacagcgtaat

SEQ ID NO: 341 - DNA, 195 bp, Sthenoteuthis oualaniensis
  source 1..195: mol_type = genomic DNA
gggacgagaagaccctaatgagcttataattttattattgttgtgcaaaacatttggtcaataatatataaattttaattggggtgattgaggaataaattagataaagtaacttccttaaattaaatatttatagataaagtaaccaatataatattgcttataatatagttaccatagggataacagcgtaat

SEQ ID NO: 342 - DNA, 187 bp, Watasenia scintillans
  source 1..187: mol_type = genomic DNA
gggacgagaagaccctaatgagctttaattttattaattatttattttaacaaatttgtagttatataaattttaattggggtgattaaggagtaaatttaagtaacctccttatttttattactgataagtaaaataaccaattttgttgcttatactagagttaccatagggataacagcgtaat

SEQ ID NO: 343 - DNA, 195 bp, Gonatopsis okutanii
  source 1..195: mol_type = genomic DNA
gggacgagaagaccctattgagcttataattttatcattattagacttattattagtgtagtagtgtataaattttaattggggtgattaaggaataaatttaatttagtaacttccttaagttttatattgttaaataaagtaaccaataagttattgcttataatatagttaccatagggataacagcgtaat

SEQ ID NO: 344 - DNA, 210 bp, Uroteuthis (Aestuariolus) noctiluca
  source 1..210: mol_type = genomic DNA
gggacgagaagaccctattgagcttataattttattagtgcttagtttaaatacaaatgcatatatataaattttaattggggtgattaaggaataaaaataataaattataagtaacttccttagaagtattagtattgataaatgaagtaaccaataaataaacatatattgcttataacatagttaccatagggataacagcgtaat

SEQ ID NO: 345 - DNA, 190 bp, Sepioteuthis australis
  source 1..190: mol_type = genomic DNA
gggacgagaagaccctaatgagcttataattgtattattattataattagatgtttataatagtttataaattttaattggggtgattaaggaatatatgtacacaacttccttaaatataatattgttggatggagtaaccaatagattattgcttataataaagttaccatagggataacagcgtaat

SEQ ID NO: 346 - DNA, 199 bp, Sepioteuthis sepioidea
  source 1..199: mol_type = genomic DNA
gggacgagaagaccctattgagcttgaattttataataattatgtatattaatagcagtaattatatataaattttaattggggtgattgaggaataatttttatgaagtaacttccttaaaataaatattgttaaattaagtacccgattaatagtatattgcttgtaatatagttaccatagggataacagcgtaat

SEQ ID NO: 347 - DNA, 334 bp, Amphioctopus kagoshimensis
  source 1..334: mol_type = genomic DNA
gcggtawtataactgtactaaggtagcataataatttgccttataaattggggctagaatgaatggtttgacgaaaattatactgtctctattttattcattagaagttaatttttatagtgagaaagcttggatgatttaaagggacgaaaagaccctattgagctttatattatattttaaatataattttttgtgtttatataaatataattttggttggggtgatcaaggaataagatatatctatgttaacttccttaaatatatatataaattaataaaccaaatttattgcttrgaagataagttaccatagggataacagcgtaat

SEQ ID NO: 348 - DNA, 198 bp, Amphioctopus membranaceus
  source 1..198: mol_type = genomic DNA
gggacgaaaagaccctattgagctttatattatattttaaatttgattttttatttatttaattatatataattttggttggggtgatcaaggaataaaatgtattttatgttaacttccttaaatggtaatatttataaatgaataaaccaaattttttgcttagaagataagttaccatagggataacagcgtaat

SEQ ID NO: 349 - DNA, 198 bp, Amphioctopus exannulatus
  source 1..198: mol_type = genomic DNA
gggacgaaaagaccctattgagctttatattattatttaaatttattatattaatttttattttaaataattttggttggggtgatcaaggaataaaatattaaatttattaacttccttatatatttaaatatatatatattaataaaccaaattttttgcttaaaagataagttaccatagggataacagcgtaat

SEQ ID NO: 350 - DNA, 200 bp, Amphioctopus rex
  source 1..200: mol_type = genomic DNA
gggacgaaaagaccctattgagctttatattattatttaaatttattatattaatttttattttaaataattttggttggggtgatcaaggaataaaatattaaatttattaacttccttatatatttaaatatatatatatattaataaaccaaattttttgcttaaaagataagttaccatagggataacagcgtaat

SEQ ID NO: 351 - DNA, 200 bp, Sepia peterseni
  misc_feature 61: note = n is a, c, g, or t
  source 1..200: mol_type = genomic DNA
gggacgagaagaccctattgagctttaaatttggaaatattatatttattaaatataatgnttgaaaaattttaattggggtgattaaggaataaaagaaataaatatattaaaatttaacttccttaaataatatattgttaaataaagtaaccaataatattgcttatataaagttaccatagggataacagcgtaat

SEQ ID NO: 352 - DNA, 219 bp, Varuna litterata
  source 1..219: mol_type = genomic DNA
ttgaagtgacgataagaccctataaaactttatataaacatattatttgattgaatttaaaataaaaatttaataataaaatttattttgttggggcgataaaagtaaaatttttattaactgctttaagttttgaatacatttatgtttgattaaaatttttagatgatcctaaataaagattaaaagtttaagttactttagggataacagcgttat

SEQ ID NO: 353 - DNA, 216 bp, Hemisquilla ensigera
  source 1..216: mol_type = genomic DNA
gggggacgataagaccctataaagctttatgataaggcctttttgtaaagaatttctattgtgaaattactagaaggtttatatcattttactggggcggtaaaagtatataattgaataactgcttttattattgaaaacaattataattgaaatacaaaatgatccattattaatgattataaaaacaagttactttagggataacagcgtaat

SEQ ID NO: 354 - DNA, 219 bp, Gonodactylus smithii
  source 1..219: mol_type = genomic DNA
ggaggacgataagaccctataaagctttataatatattttctgttatataaattgttaattgttaacttattaaagaaataatattattttactggggcggtaaaagtatataatttagggtaactgcttttgttattataaacaatgataattgaatgatatattgatccattattagtgagcataaggacaagttactttagggataacagcgtaat

SEQ ID NO: 355 - DNA, 214 bp, Pullosquilla thomassini
  source 1..214: mol_type = genomic DNA
aagggacgataagaccctataaagctttataacataatctttttgcgcaaaatatctaatgtaaaacattaaaagaatcatgttattttactggggcggtaaaagtatattaaaaataactgcttttgttttataaaacaataataattggtacacagattgatccattaataatgatcataaaaacaagttactttagggataacagcgtaat

SEQ ID NO: 356 - DNA, 216 bp, Chorisquilla trigibbosa
  source 1..216: mol_type = genomic DNA
gagggacgataagaccctataaagctttatgatattttctttttatattagttagtaaaatgtagtttattaaaagaaaaatattattttactggggcggtagaagtatatttttgattaactgcttctgttttgtaagtcaattataattgaaatatgaattgatccattattaatgatcataagaacaagttactttagggataacagcgtaat

SEQ ID NO: 357 - DNA, 213 bp, Chionoecetes opilio
  source 1..213: mol_type = genomic DNA
gggggacgataagaccctataaagctttacatataagtagagtttacttaattaaaaagtaaaagtttaatctaattaatgtgttttgttggggcgacataaatataatttatattaactgtttataaattaatacaataataaatgattttaatgtagttgatccttattaaagattaaaagactaagttactttagggataacagcgttat

SEQ ID NO: 358 - DNA, 210 bp, Chionoecetes opilio
  source 1..210: mol_type = genomic DNA
aggggacgataagaccctataaagctttacatatattataagtttgattaaattaaaatataaaaatctaaattatatattatgttttgttggggcgacaaaaatataagtaataataactgtttaagaaaaaaacaataataaatgaataattaaatgatccttggttaaagattaaagatcaagttactttagggataacagcgttat

SEQ ID NO: 359 - DNA, 210 bp, Telmessus acutidens
  source 1..210: mol_type = genomic DNA
aggggacgataagaccctataaagctttacatatattataaatttgattaaattaaaatctaaaaatctaaattatatattatgttttgttggggcgacaaaaatataagtaataataactgtttaataaaaaaacaataataaatgaataattaaatgatccttatttaaagattaaagatcaagttactttagggataacagcgttat

SEQ ID NO: 360 - DNA, 216 bp, Lithodes aequispinus
  source 1..216: mol_type = genomic DNA
caaaaagacgataagaccctataaatctttacaataaatatattttatattttagcttataagtgtataagaaataaaaatataggtttgttgcgctggggcggcgtagatatataaataaactgtctataatttaaatacaataatcattgcttaatacaaattgatccttaaatagattaaaagattaagatactttagggataacagcgttat

SEQ ID NO: 361 - DNA, 219 bp, Panulirus echinatus
  source 1..219: mol_type = genomic DNA
gggggacgataagaccctataaatcttgataataagactaataacttcatatattagttgtgagtctgaggttaatacttttattattttgttggggcgacagaagtataattttagtaactgctttggcttaatagttaattatttttataatgtttattggttaagatccttctgtgaggatgccagattaagttactttagggataacagcgtaat

SEQ ID NO: 362 - DNA, 217 bp, Jasus paulensis
  misc_feature 32: note = n is a, c, g, or t
  misc_feature 62: note = n is a, c, g, or t
  source 1..217: mol_type = genomic DNA
gggggacgataagaccctataaatctttatgntagttagtaataacattaaaaattaatttnacatttttttgttagtagttttagcattttgctggggcggcaggggtataatttaataactgctctagagttgtagttaattatgtttaaattattggtagttgatcctttagtggagattatagattaagttactttagggataacagcgcaat

SEQ ID NO: 363 - DNA, 217 bp, Jasus caveorum
  source 1..217: mol_type = genomic DNA
gggggacgataagaccctataaatctttatgttagttagtaataacattaaaaattaattttacatttttttgttagtagttttagcattttgctggggcggcaggggtataatttaataactgctctagagttgtagttaattatgtttaaattattggtagctgatcctttagtggagattatagattaagttactttagggataacagcgcaat

SEQ ID NO: 364 - DNA, 208 bp, Parastacus pilimanus
  source 1..208: mol_type = genomic DNA
aagggacgataagaccctataaagcttcacattaagtatttaaaattttaagtgagtaataaaattttattagatgtaatatgttttgttggggcgacaggaatataagagatttaactgttctagataagttaatcaaaaatacttgatctttaaggactttttattaaagtattagactaagttactttagggataacagcgttat

SEQ ID NO: 365 - DNA, 207 bp, Parastacus brasiliensis
  source 1..207: mol_type = genomic DNA
aagggacgataagaccctataaagcttcacattaagtctttaaaattttaattaagtaataaaattttattaaatgcaatatgttttgttggggcgacaggagtataagaaatttaactgctctaaataataagtcaaagatacttgatctttggggactttttattaaagtgttagattaagttactttagggataacagcgtaat

SEQ ID NO: 366 - DNA, 207 bp, Parastacus defossus
  source 1..207: mol_type = genomic DNA
aagggacgataagaccctataaagctttacattaagcttttaaaattttaattaagtaataaaattttattaaatgcaatatgttttgttggggcgacaggaatataagaaatttaactgttctaaataataaatcaaagatacttgatctttggggactttttgttaaagtgttagattaagttactttagggataacagcgttat

SEQ ID NO: 367 - DNA, 209 bp, Parastacus nicoleti
  source 1..209: mol_type = genomic DNA
aagggacgataagaccctataaagctttacattcaactttttaggattattaattaagtaataaaagtttgttaggtataatatgttttgttggggcgacaggaataaaagagatttaactgttctaaataataaatcagaaatatttgatttttaatgattttttattaaaatattagattaagttactttagggataacagcgttat

SEQ ID NO: 368 - DNA, 216 bp, Gonodactylus graphurus
  source 1..216: mol_type = genomic DNA
gagggacgataagaccctataaagctttatagtagttcctttttattcaaattataaaatgttaaccttttgaaagattactactattttactggggcggtagaagtatatttatgattaactgcttttgttttgacaaacaataataattgaatatttatgtgatccattattaatgatcaaaagtacaagttactttagggataacagcgtaat

SEQ ID NO: 369 - DNA, 217 bp, Jasus lalandii
  source 1..217: mol_type = genomic DNA
gggggacgataagaccctataaatctttatactagttagtaataacattaaagattaattttacatttttttgttagtagttttagtattttgctggggcggcaggggtataatttaataactgctctagagttgtagttaattatgtttaaattgttggtagttgatcctttagtggagatcatagattaagttactttagggataacagcgcaat

SEQ ID NO: 370 - DNA, 217 bp, Jasus paulensis
  source 1..217: mol_type = genomic DNA
gggggacgataagaccctataaatctttatgttagttagtaataacattaaaaattaattttacatttttttgttagtagttttagcattttgctggggcggcaggggtataatttaataactgctctagagttgtagttaattatgtttaaattattggtagttgatcctttagtggagattatagattaagttactttagggataacagcgcaat

SEQ ID NO: 371 - DNA, 211 bp, Lopholithodes mandtii
  source 1..211: mol_type = genomic DNA
aagacgatgagaccctataaatctttacaataaatatattttatattttagcttataagtatataaaaaataaaatatagacttgttgcgctggggcggcgtagatatataaataaactgtctatagtttaaatacaataatcattgcttaatacaaattgatccttaaatagattaaaagattaagatactttagggataacagcgttat

SEQ ID NO: 372 - DNA, 216 bp, Lithodes santolla
  source 1..216: mol_type = genomic DNA
caaaaagacgataagaccctataaatctttacaataaatatattttatattttaacttataagtgtataagaaataaaaatatagatttgtttcgctggggcggcgtagatatataaataaactgtctataatttaaatacagtaattattgcttaatataaattgatccttaaatagattaaaagattaagatactttagggataacagcgttat

SEQ ID NO: 373 - DNA, 216 bp, Lithodes maja
  source 1..216: mol_type = genomic DNA
caaaaagacgataagaccctataaatctttacaataaatatattttatattttagcttataagtgtataaaaaataaaaatatagatttgttgcgctggggcggcgtagatatataaataaactgtctataatttaaatacaataatcattgtttaatatagattgatccttaaatagattaaaagattaagatactttagggataacagcgttat

SEQ ID NO: 374 - DNA, 217 bp, Jasus edwardsii
  source 1..217: mol_type = genomic DNA
gggggacgataagaccctataaatctttatactagttagtaataacattaaagattaattttacatttttttgttagtagttttagtattttgctggggcggcaggggtataatttaataactgctctagagttgtagttaattatgtttaaattgttggtagttgatcctttagtggagattatagattaagttactttagggataacagcgcaat

SEQ ID NO: 375 - DNA, 217 bp, Panulirus regius
  source 1..217: mol_type = genomic DNA
gggggacgataagaccctataaatctttatatttcggtcagtaaattttagagattagttatacatttagtgttattgaactcaaatattttgttggggcgacaggagtataataagtcactgctttgtttataagttaattatgtttattttgaaaaccaatattgatcctttctagagaaatcagactaagttactttagggataacagcgtaat

SEQ ID NO: 376 - DNA, 217 bp, panulirus pascuensis
  source 1..217: mol_type = genomic DNA
gggggacgataagaccctataaatcttgataaccaaagtaataattttatgtagtagtcataagcttaaatttaatacttttgttattttgttggggctacagggggtataattaataactgcggttgggaaaaataattaattatttttattgtgttgtgtttaagatccttctttgaagatacaggtcaagttactttagggataacagcgtaat

SEQ ID NO: 377 - DNA, 217 bp, Panulirus laevicauda
  source 1..217: mol_type = genomic DNA
gggggacgataagaccctataaatctttatattttagtcgataagttttaggaattagttacacacttagtatcattggactaaaatattttgttggggcgacagaagtataataagtaactgctttgtttataaattaattatttttattatgagagctgatatagatcctctttagagaaatcagattaagttactttagggataacagcgtaat

SEQ ID NO: 378 - DNA, 219 bp, Panulirus gracilis
  source 1..219: mol_type = genomic DNA
gggggacgataagaccctataaatctttatgttttggttaataaattttagaaattagttatatatttagtattattaggctaaaatattttgttggggcgacaggagtataataagtaactgcttcggtttgtaaattaattatttttatattggaaatgtaatattgatcctttttagagaaatcagactaagttactttagggataacagcgtaat

SEQ ID NO: 379 - DNA, 218 bp, Panulirus guttatus
  source 1..218: mol_type = genomic DNA
gggggacgataagaccctataaatcttgatagagaagattataaccttagaaagtagttacagttctcgggttaagacattttctattttgttggggcgacaggagtataattagtaactgctttgggcttaatatgtaattatcgctattgtgggtattatgagagatccttctttgaagatgtcagactaagttactttagggataacagcgtaat

SEQ ID NO: 380 - DNA, 216 bp, Panulirus inflatus
  source 1..216: mol_type = genomic DNA
gggggacgataagaccctataaatctttatgttttaactaataaattttagggattagttatatacctagtattattagttgaaatattttgttggggcgacaggagtataataagtaactgctttgtttataaaattaattgtttttattgtggaaattggtatgatcctctttagagaaatcagactaagttactttagggataacagcgtaat

SEQ ID NO: 381 - DNA, 217 bp, Panulirus femoristriga
  source 1..217: mol_type = genomic DNA
gggggacgataagacctataaatcttgataaccaaagtaataatttttatgtagtagtcataagctttaaatttaatacttttgttattttgttggggcgacaggggtataattagtaactggctttgagtaaagaattaattatttttattgtgttgtgtttgagatccttctttgaagatatcagatcaagttactttagggataacaggtaatc

SEQ ID NO: 382 - DNA, 213 bp, Chionoecetes bairdi; identical to SEQ ID NO: 357
  source 1..213: mol_type = genomic DNA

SEQ ID NO: 383 - DNA, 215 bp, Paralomis granulosa
  source 1..215: mol_type = genomic DNA
aaaagacgataagaccctataaatctttacaagaaatatattttatgttttagcttataagtaatataagaaataaaaatataagtttgttgcgctggggcggcgtagatatataaataaactgtctatgatttaaatacagtaattattgcttaatacaaattgatccttaaatagattaaaagattaagatactttagggataacagcgttat

SEQ ID NO: 384 - DNA, 207 bp, Scyllarus arctus
  source 1..207: mol_type = genomic DNA
gggggacgataagaccctataaatctttatactcggctttattgagcaaaatactagaataatttttgttcgataggtttgagtattttgttggggcgacaggagtataaaatgtaactgctttaaaataatagttaattgatgtttagttaagcgatcctttaataaagatgttagtttaagttactttagggataacagcgtaat

SEQ ID NO: 385 - DNA, 219 bp, Palinurus elephas
  source 1..219: mol_type = genomic DNA
aggggacgataagaccctataaatctttatatttgtcataatgatattaagtattataattgaaaatttagttttattgggatagaatattttgttggggcgacaggagtataaaaaagtaactgctttgtaagatgagttaataatttttaatgtgtgatatagttgatccttcagtgaggatttcagaataagttactttagggataacagcgtaat

SEQ ID NO: 386 - DNA, 221 bp, Episesarma mederi
  source 1..221: mol_type = genomic DNA
aaaagacgataagaccctataaaacttaatataagttaattgtttagtagaatttttaataataaatatttaataattagtttttattttattggggtgataatggtaaaatgattattaactgttgattattttataacaaaaattattgaataaatatataataaatgatccttgttaaagattaaaagtttaagttactttagggataacagcgttat

SEQ ID NO: 387 - DNA, 198 bp, Austropotamobius torrentium
  source 1..198: mol_type = genomic DNA
gggggacgataagaccctataaaactttatattttgaaataataattaattttgtgtgggagatttatttttaagtattttattggggtgataaggataaaataataaataactgtctttttttattacagtaatatttgagcaaatgaacctagtaagggaaagaagattaagttactttagggataacagcgtaat

SEQ ID NO: 388 - DNA, 221 bp, Cycloachelous granulatus
  source 1..221: mol_type = genomic DNA
aggggacgataagaccctataaagctttataaattcaggaattttagatattttatatataaatctattttttgataatttatttggttggggcgacaagggtataattataaataactgccattaatgtttatacatatatttttgatttaaatttgatatagtagttgatcctttttaaggatttaaagatcaagttactttagggataacagcgtaat

SEQ ID NO: 389 - DNA, 217 bp, Eriocheir recta
  source 1..217: mol_type = genomic DNA
aagggacgataagaccctataaagcttaatataaaattttatttagtcaaattttacgttataaagactttgtaagtatgagtttattttattggggcgataagagtaaaatgattgttaactgcttaatattaaatacacttatgagtgattaaatttttagatgatcctaattaaagattagaagtttaagttactttagggataacagcgttat

SEQ ID NO: 390 - DNA, 209 bp, Cervimunida johni
  source 1..209: mol_type = genomic DNA
aagggacgataagaccttataaatctttatattaatttgttttttgtcttttaataatgtaatgttgataataaaataaagtatattgtgttggggtgatgagaatataaaattaactgttctaaattaaacacaaatttatttgtataaaataatagatccttattaaagattaaaaggttaagttactttagggataacagcgttat

SEQ ID NO: 391 - DNA, 212 bp, Achelous floridanus
  source 1..212: mol_type = genomic DNA
gggggacgataagaccctataaagctttataaaagtagtaagatcacatatttgaatataaaagcgattatataatcttttattttgttggggcgacagtagtataattgaaagtaactgctattatgaaaacacattgatttatgattatttattaatggatcccttttaaggattataaaatcaagttactttagggataacagcgttat

SEQ ID NO: 392 - DNA, 214 bp, Portunus sayi
  source 1..214: mol_type = genomic DNA
gagggacgataagaccctataaagctttataaatttaaagattttacatattttatctataatggtaatctctggagaatttattgggttggggcaacaaaagtataattatcaataactgctataaaataatacaattatctttgtgtgggtaaattagttgatccctgaaaaggattaaaagatcaagttactttagggataacagcgtaat

SEQ ID NO: 393 - DNA, 210 bp, Portunus anceps
  source 1..210: mol_type = genomic DNA
gagggacgataagaccctataaagctttataaactaaaagaattcacaaatttagacataaaggtaattttctaacttcttattttgttggggcgacagtagataactaaagtaactgctattataggagtacaatgatttttgattagccagtaacagatccctactaaggattataaaaccaagttactttagggataacagcgttat

SEQ ID NO: 394 - DNA, 218 bp, Palinurus gilchristi
  source 1..218: mol_type = genomic DNA
gggggacgataagaccctataaatctttatatttattatgataacattaaatattataattgaaaatttagttttactggaataaaatattttgttggggcgacaggagtataaaaaataactgctttgtaatatgggttaataatttttaatatgtagtatagttgatccttcagtgaggattttagaataagttactttagggataacagcgtaat

SEQ ID NO: 395 - DNA, 218 bp, Palinurus mauritanicus
  source 1..218: mol_type = genomic DNA
gggggacgataagaccctataaatctttatatttattatgataacattaaatattataattgaaaatttaattttactgtaataggatattttgttggggcgacaggagtataaaaaataactgctttgtaagatgggttaataatttttaatgtgtagtatatttgatccttcagtgaggattttagaataagttactttagggataacagcgtaat

SEQ ID NO: 396 - DNA, 218 bp, Palinurus charlestoni
  source 1..218: mol_type = genomic DNA
gggggacgataagaccctataaatctttatatttattatgataacattaaatattataattgaaaatttagttttactggaataaaatattttgttggggcgacaggagtataaaaaataactgctttataagataggttaataatttttaatatgtagtatagttgatccttcagtgaggattttagaataagttactttagggataacagcgtaat

SEQ ID NO: 397 - DNA, 219 bp, Pseudosquilla ciliata
  source 1..219: mol_type = genomic DNA
agtgacgataagaccctatgaaagctttatgagtgtgcttcttttatatgtaaaatgaatttaatattaacatattaaagaaatcacattattttactggggcggtaaaagtataaatgattaactgcttttgttatgcaaaacaatgataattggtatgtaaattgatccattaataatgattataaaaacaagttactttagggataacagcgtaat

SEQ ID NO: 398 - DNA, 210 bp, Pleuroncodes monodon
  source 1..210: mol_type = genomic DNA
aaggggcgataagaccctataaatctttatattaaattgtttttagtttattaataatgtaatttaaataataaggcaaaatatattgtgttggggtgatgggaatataaaaaattaactgttctgaaataaaaacaaatttatttgatttaataatagatcctttttaaagattaaaagtttaagttactttagggataacagcgctat

SEQ ID NO: 399 - DNA, 212 bp, Portunus ventralis
  source 1..212: mol_type = genomic DNA
aggggacgataagaccctataaagctttataaatctataaattattacatatttaaaatataaaggtaatcttttagatatttatttggttggggcgacagtggtataaattaattaactgctaataaaagttacaataatttttgattcttaagttatagatcctctctagggattaaaaaatcaagttactttagggataacagcgttat

SEQ ID NO: 400 - DNA, 209 bp, Achelous spinicarpus
  source 1..209: mol_type = genomic DNA
gggggacgataagaccctataaagctttataaatttatagactttatatatttaaaataaaaataatctctaaaacatttatttggttggggcgacaaaagtataatttcaaataactgctattaaattaacaataatttttgtttatgaataattgatccttctatgaggagtaaaagttcaagttactttagggataacagcgtcat

SEQ ID NO: 401 - DNA, 212 bp, Callinectes toxotes
  source 1..212: mol_type = genomic DNA
gagggacgataagaccctataaagctttataaatttattaatttcgtttattttttattataataatgatgataagaaaatttatttggttggggcaaccgaagtataattttaaataactgctataatttaatacaatgatttttgttttatataataattgatcctctaagagattaaagaccaagttactttagggataacagcgtaat

SEQ ID NO: 402 - DNA, 209 bp, Callinectes danae
  source 1..209: mol_type = genomic DNA
gagggacgataagaccctataaagctttataaatttattaatttcttctatttttttataatgaggatgttaagaagatttatttggttggggcaaccgaagtataattaaagataactgctaatagtttatacaatgatttttgttttaataataattgatccttaaaaagattaaagaccaagttactttagggataacagcgtaat

SEQ ID NO: 403 - DNA, 210 bp, Callinectes ornatus
  source 1..210: mol_type = genomic DNA
gagggacgataagaccctataaagctttataaatttattgatttcgtctatttttcttataataatgatattgagaagatttatttggttggggcaaccgaagtataattaaagataactgctactattttatacaatgatttttgtttaaatgataattgatccttaaaaagattaaagaccaagttactttagggataacagcgtaat

SEQ ID NO: 404 - DNA, 211 bp, Callinectes marginatus
  source 1..211: mol_type = genomic DNA
gagtgacgacaagaccctataaagctttataaatttatttatttcgtctattttcttttataatgatgatgttaagaaggtttatttggttggggcaaccagagtataattaaagataactgctacaatttaatacaatgatttttgtttatgaaataattgatccttgaaaagattaaagatcaagttactttagggataacagcgtaat

SEQ ID NO: 405 - DNA, 212 bp, Callinectes affinis
  source 1..212: mol_type = genomic DNA
gagggacgataagaccctataaagctttataaatttattaatttcgtttattttctattataataatgataataagaagatttatttggttggggcaaccgaagtataagtttaaataactgctataatttaatacaatgatttttgttttataaaataattgatcctcgaagagattaaagaccaagttactttagggataacagcgtaat

SEQ ID NO: 406 - DNA, 211 bp, Callinectes rathbunae
  source 1..211: mol_type = genomic DNA
gagggacgataagaccctataaagctttataaatttattaatttcgtttattttttactataatgatgataataagaagatttatttggttggggcaaccgaagtataagtttaaataactgctataatttaatacaatgatttttgttttaaaaataattgatcctcgaagagattaaagaccaagttactttagggataacagcgtaat

SEQ ID NO: 407 - DNA, 212 bp, Callinectes bocourti
  source 1..212: mol_type = genomic DNA
gagggacgataagaccctataaagctttataaatttattaatttcgtttattttctattataataatgataataagaagatttatttggttggggcaaccgaagtataagtttaaataactgctataatttaatacaatgatttttgttttaaaaaataattgatcctcgaagagattaaagaccaagttactttagggataacagcgtaat

SEQ ID NO: 408 - DNA, 209 bp, Callinectes similis
  source 1..209: mol_type = genomic DNA
gagggacgataagaccctataaagctttataaatttattaatttcttctatttttttataatgatgatgttaagaagatttatttggttggggcaaccgaagtataattaaagataactgctaatattttatacaatgatttttgttttaataataattgatccttaaaaagattaaagaccaagttactttagggataacagcgtaat

SEQ ID NO: 409 - DNA, 210 bp, Callinectes bellicosus
  source 1..210: mol_type = genomic DNA
gagggacgataagaccctataaagctttataaatttattaatttcgtctatttttttataatgatgatgttgaaaagatttatttggttggggcaaccgaagtataatttttaataactgctataacttaatacaatgatttttgtttgttattataattgatccttaaaaagattaaagaccaagttactttagggataacagcgtaat

SEQ ID NO: 410 - DNA, 210 bp, Callinectes arcuatus
  source 1..210: mol_type = genomic DNA
gagggacgataagaccctataaagctttataaatttattaattttgtctatttttcttataatgatgatgttaagaagatttatttggttggggcaaccgaagtataattaaagataactgctactattttatacaatgatttttgttttaataataattgatccttaaaaagattaaagaccaagttactttagggataacagcgtaat

SEQ ID NO: 411 - DNA, 211 bp, Metanephrops armatus
  source 1..211: mol_type = genomic DNA
gagggacgataagaccctataaagcttgataattttatgtataaataaataagttgttagtgttatattgtttatctgtaaaattatttcgttggggcgacgataatataatttgtaactgtttgggggttagattcaaatatgtttgtgtgttaatgatccattaattgttgattaaaaatttaagttactttagggataacagcgttat

SEQ ID NO: 412 - DNA, 211 bp, Metanephrops mozambicus
  source 1..211: mol_type = genomic DNA
gagggacgataagaccctataaagcttgataattttatatgtaaataaataagttgttagtgttatattgtttacttacaaaattatttcgttggggcgacgataatataatttgtaactgtttgaggtttagatccaaatatatttgtgtttaagtgatccattaattgttgattaaaaatttaagttactttagggataacagcgttat

SEQ ID NO: 413 - DNA, 211 bp, Metanephrops japonicus
  source 1..211: mol_type = genomic DNA
gagggacgataagaccctataaagcttgataattttatatataaataaataagttgttagtgttatattgtttatctgtaaaattatttcgttggggcgacgataatataatttgtaactgtttgggggttagattcaaatatgtttgtgtgttaatgatccattaattgttgattagaaatttaagttactttagggataacagcgttat

SEQ ID NO: 414 - DNA, 211 bp, metanephrops andamanicus
  source 1..211: mol_type = genomic DNA
gagggacgataagaccctataaagcttgataattttatatgtaaataaataagttgttagtgttatattgtttatttataaaattatttcgttggggcgacgataatataatttgtaactgtttgaggtttagatccaaatatatttgtgtttaaatgatccattaattgttgattaaaaatttaagttactttagggataacagcgttat

SEQ ID NO: 415 - DNA, 211 bp, Metanephrops velutinus
  source 1..211: mol_type = genomic DNA
gagggacgataagaccctataaagcttgataattttatatgtaaataaataagttgttagtgttatattgtttatttataaaattatttcgttggggcgacgataatataatttgtaactgtttgaggtttagatccaaatatatttgtgtttaaatgatccattaattgttgattaaaaatttaagttactttagggaaaacagcgttat

SEQ ID NO: 416 - DNA, 211 bp, Metanephrops sagamiensis; identical to SEQ ID NO: 414
  source 1..211: mol_type = genomic DNA

SEQ ID NO: 417 - DNA, 213 bp, Metanephrops binghami
  source 1..213: mol_type = genomic DNA
aagggacgataagaccctataaagcttaataatttttgtaagtaaaataaataaattgttagtgatatgctgcttacttactaaattatttcgttggggtgacgaggatataattttgtaactgttttagtttaaattcagggatacttgtactcaatgatccatttatttgatgattaaaaatttaagttactttagggataacagcgttat

SEQ ID NO: 418 - DNA, 207 bp, Parastacus pugnax
  source 1..207: mol_type = genomic DNA
aagggacgataagaccctataaagctttacattaagcgtttaaaattttaattaagtaataaaattttattaaatgtaatatgttttgttggggcgacaagaatataaaaaatttaactgttctagataataagtcagaaatatctgatctttgaggactttttattaaagtattagattaagttactttagggataacagcgttat

SEQ ID NO: 419 - DNA, 204 bp, Paranephrops zealandicus
  source 1..204: mol_type = genomic DNA
gagggacgataagaccctataaagctttacattgaatttattaaaaagtaatttaaataataaaagtttattagtaaggagatgttttgttggggtgacaagaatataataaatataactgttcttttttagttcaaagatatttgaaaaggagatccttaataaagatgttagagtaagttactttagggataacagccgtaa

SEQ ID NO: 420 - DNA, 209 bp, Callinectes exasperatus
  source 1..209: mol_type = genomic DNA
gagggacgataagaccctataaagctttataaatttattaatttcgtttatttttttataatgatgatgttaagaaagtttatttggttggggcaacctaagtataattaaagataactgctacgattttatacaatgatttttgttttaatagtaattgatccttgaaaagattaaagaccaagttactttagggataacagcgtaat

SEQ ID NO: 421 - DNA, 218 bp, Palinurus barbarae; identical to SEQ ID NO: 394
  source 1..218: mol_type = genomic DNA

SEQ ID NO: 422 - DNA, 217 bp, Sagmariasus verreauxi
  source 1..217: mol_type = genomic DNA
gggggacgataagaccctataaatctttactctggagacaataacattgaagattactgctaataattccgtgttattgtttttagtgttttgttggggcgacaggagtataaataaataactgctttaaacgtatagttaattatagttaagttgttgataagtgatcctttagtatggattatagattaagttactttagggataacagcgtaat

SEQ ID NO: 423 - DNA, 210 bp, Metanephrops rubellus
  source 1..210: mol_type = genomic DNA
aagggacgataagaccctgtaaagcttaataattctacgagtggataaataaattgtttagtggcaatgttactcacttgttaaattatttcgttggggtgacgaggatataatttgtaactgtctaaatttaaagtcaaggatgcttgtattcgatgatccataattggtgattaaaaatttaagttactttagggataacagcgttat

SEQ ID NO: 424 - DNA, 210 bp, Metanephrops challengeri
  source 1..210: mol_type = genomic DNA
gagggacgataagaccctataaagcttgataattttatatataattaaataaattattagtattatattatttatatattaaattatttcgttggggcgacgataatataatttgtaactgtttgaaattttaattcagaaatatttgtttttaatgatccttttattattgattaaaaatttaagttactttagggataacagcgttat

SEQ ID NO: 425 - DNA, 211 bp, Metanephrops neptunus
  source 1..211: mol_type = genomic DNA
gagggacgataagaccctataaagcttaataattttgtatatagataaataagttgttagtattatattgtctatttattaaattatttcgttggggcgacgataatataatttgtaactgtttgaaatatttaattcagaagtatttgtatttaatgatccttttgttgttgattaaaaatttaagttactttagggataacagcgttat

SEQ ID NO: 426 - DNA, 210 bp, Metanephrops australiensis
  source 1..210: mol_type = genomic DNA
aagggacgataagaccctataaagcttgataattttatatataagtaaataagttgttagtattatattgtttatatataaaattatttcgttggggcgacgataatataatttgtaactgtttaatgttttgattcaaatatatttgtgtttaatgatccattaattgttgattaaaaatttaagttactttagggataacagcgttat

SEQ ID NO: 427 - DNA, 210 bp, Metanephrops arafurensis
  source 1..210: mol_type = genomic DNA
aagggacgataagaccctataaagcttgataattttatatataagtaaataagttgttagtgttatattgtttatatataaaattatttcgttggggcgacgataatataatttgtaactgtttaatgttttgattcaaatatatttgtgtttaatgatccattaattgttgattaaaaatttaagttactttagggataacagcgttat

SEQ ID NO: 428 - DNA, 209 bp, Metanephrops boschmai
  source 1..209: mol_type = genomic DNA
gagggacgataagaccctataaagcttgataattttatatataaataaataggttgttagtgttatattgtctatttataaaattatttcgttggggcgacgatgatataatttgtaactgtttaaggttttgattcaaatatatttgtatttaatgatccatttttgttgattaaaaatttaagttactttagggataacagcgttat

SEQ ID NO: 429 - DNA, 210 bp, Metanephrops formosanus
  source 1..210: mol_type = genomic DNA
gagggacgataagaccctataaagcttgataattttatatataaataaataagttgttagtgttatatcgtttatttataaaattatttcgttggggcgacgataatataatttgtaactgtttaggtttagattcaaatatatttgtgttttaatgatccattaattgttgattaaaaatttaagttactttagggataacagcgtaat

SEQ ID NO: 430 - DNA, 209 bp, Metanephrops sinensis
  source 1..209: mol_type = genomic DNA
gagggacgataagaccctataaagctagataattttgtatttaaataaataagttgttagtgttatatcatttatttataaaattatttcgttggggcgacgataatataatttgtaactgtttaggtttagattcaaatatatttgtgttttatgatccattaattgttgattaaaaatttaagttactttagggataacagcgttat

SEQ ID NO: 431 - DNA, 216 bp, Lithodes ferox
  source 1..216: mol_type = genomic DNA
caaaaagacgataagaccctataaatcttaacaataaatatattttatattttagtttataagtgtataaggaataaaaatataggtttgttacgctggggcggcgtagatatataaataaactgtctatagtttaaatacaataatcattgcttaatacaaattgatccttaaatagattaaaagattaagatactttagggataacagcgttat

SEQ ID NO: 432 - DNA, 216 bp, Oratosquillina interrupta
  source 1..216: mol_type = genomic DNA
gagggacgataagaccctataaagctttatgttacatttcttttttataaattgtattgttattaacttattttagaatttgtatcattttactggggcggtaaaagtataataaagattaactgcttttgttttgaataacaattttagttggaaataaattgatccattattaatgatcataagaacaagttactttagggataacagcgtaat

SEQ ID NO: 433 - DNA, 216 bp, Odontodactylus japonicus
  source 1..216: mol_type = genomic DNA
gagggacgataagaccctataaagctttatagtgtaattatttaatatgaaatatattaacttgaaattatttaataaaaagcactattttactggggcggtaaaagtataacttaaactaactgcttttatttttaaaaacaattataattgaatataaattgatccattattaatgatcacaagaacaagttactttagggataacagcgttat

SEQ ID NO: 434 - DNA, 215 bp, Miyakella nepa
  source 1..215: mol_type = genomic DNA
gagggacgataagaccctataaagctttatggtatttttcttttttataaattgtattgttattaacttattttagaatttttatcattttactggggcggtaaaagtataattagattaactgcttttgttttaaataacaattttagttggaaataaattgatccattattaatgatcataagaacaagttactttagggataacagcgtaat

SEQ ID NO: 435 - DNA, 218 bp, Erugosquilla woodmasoni
  source 1..218: mol_type = genomic DNA
gagggacgataagaccctataaagctttatgatacttttcttttttacaaattatctcgttattaacttattttagaattaatatcattttactggggcggtaaaagtataataaaaagattaactgcttttattttgttgaacaatgttaattggaaataaactgatccattattaatgatcataagaacaagttactttagggataacagcgtaat

SEQ ID NO: 436 - DNA, 217 bp, Clorida decorata
  source 1..217: mol_type = genomic DNA
aagggacgataagaccctataaagctttataatatatttcttttacataaattgttttgttattaacgtgttttagattatatattattttactggggcggtaaaagtataataaaagattaactgcttttattttgtgtaacaattgtagttggaaataaattgatccattattaatgattataaaaacaagttactttagggataacagcataat

SEQ ID NO: 437 - DNA, 217 bp, Dictyosquilla foveolata
  source 1..217: mol_type = genomic DNA
gagggacgataagaccctataaagctttatggtattcttcttttttataaattgttttgttattaacttattttagaatttttatcattttactggggcggtaaaagtataataaaagattaactgcttttattttgtgtaacaattttatttggaaataaattgatccattattaatgattataagaacaagttactttagggataacagcgttat

SEQ ID NO: 438 - DNA, 216 bp, Anchisquilla fasciata
  source 1..216: mol_type = genomic DNA
gagggacgataagaccctataaagctttatagtatatttcttttgtataaattattttgttactaacatattttagaacatatattattttactggggcggtaaaagtataataaagactaactgcttttattttgaataacaattgtagttggaaataaattgatccattattaatgattataaaaacaagttactttagggataacagcgtaat

SEQ ID NO: 439 - DNA, 215 bp, Scyllarides herklotsii
  source 1..215: mol_type = genomic DNA
gagggacgataagaccctataaatctttatattttgggttataaggttaaaaattaggttttcatttaattttataaagttaaatattttgttggggagacagaggtataaagaaagataactgccttaaaatttatagttaagaatagttaagatagaaagtgatcctttaataaggaaattagaataagttactttagggataacagcgtaat

SEQ ID NO: 440 - DNA, 218 bp, Palinurus delagoae; identical to SEQ ID NO: 394
  source 1..218: mol_type = genomic DNA

SEQ ID NO: 441 - DNA, 201 bp, Astacus astacus
  source 1..201: mol_type = genomic DNA
taaagggacgataagaccctataaaactttatatttaaaaataattgctattattatttaaagagtttatttttaaatattttattggggtgataaggataaaatataagacaactgtcttttattttaacaataatttttgagtaaatgatcttaataaaggaataaaagattaagttactttagggataacagcgtaat

SEQ ID NO: 442 - DNA, 213 bp, Portunus hastatus
  source 1..213: mol_type = genomic DNA
gggggacgataagaccctataaagctttataaatccataaattattatatatttaagatgataaaagtaatcttttagatatttatttggttggggcgacagtggtataaatcaattaactgctaataaaaattacaataatttttgatttctaagttatagatcctctttagggattaaaaaatcaagttactttagggataacagcgttat

SEQ ID NO: 443 - DNA, 213 bp, Achelous ordwayi
  source 1..213: mol_type = genomic DNA
gggggacgataagaccctataaagctttataaatctacaaattttatatatttgtgaataaaaataatttttctaatatttatttggttggggcgacaaaagtgtaattaaaataaccgctaataatttgttacaatgatttttgtttggcgaagttaatggatcccttttaaggattaaaaaatcaagttactttagggataacagcgttat

SEQ ID NO: 444 - DNA, 212 bp, Carcinus maenas
  source 1..212: mol_type = genomic DNA
gggggacgataagaccctataaaactttatatatgcaacaatacagttgaattaaagcataaaaacttattcgataaaatatatttagttggggcgactgaggtataatttatagtaactgattaaaagcaagacaaataatatttgattaactttaattgatcctttttaaagattaaaagattaagttactttagggataacagcgtaat

SEQ ID NO: 445 - DNA, 213 bp, Portunus inaequalis
  source 1..213: mol_type = genomic DNA
gggggacgataagaccctataaagctttataaatctataaattattatatatttaaaataataaaagtaattttttagatatttatttggttggggcgacagtggtataaattaattaactgctaataaaaattacaataatttttgatttttaagtcatagatcctctatagggattaaaaaatcaagttactttagggataacagcgttat

SEQ ID NO: 446 - DNA, 203 bp, Astacoides madagascariensis
  source 1..203: mol_type = genomic DNA
aagggacgataagaccctataaaactttatattagaattatttaaaaactatttagatagaaaggtaagtaagtagtaaaatattttgttggggcgacaagaatataagaatataactgttcttaagtataatcaaaaatttttgatttaatgatcctaggtaaagatattagaataagttactttagggataacagcgtaat

SEQ ID NO: 447 - DNA, 210 bp, Erimacrus isenbecki
  source 1..210: mol_type = genomic DNA
aggggacgataagaccctataaagctttacatacattataagtttgatttaagcaaaatttaaaaatctaaattatatattatgttttgttggggcgacaaaaatataagtaataataactgtttaataaaaaaacaataataaatggacaattaaatgatccttgtttaaagattaaagaccaagttactttagggataacagcgttat

SEQ ID NO: 448 - DNA, 217 bp, Hemisquilla australiensis
  source 1..217: mol_type = genomic DNA
gggggacgataagaccctataaagctttatgataaggcctttttgtaaagaatttctattgtgaaattactaagggggtttatattattttactggggcggtaaaagtatataattgaataactgcttttattattgaaaacaattataattgaaatacaaaatgatccattattaatgattataaaaacaagttactttagggataacagcgtaat

SEQ ID NO: 449 - DNA, 215 bp, Austrosquilla tsangi
  source 1..215: mol_type = genomic DNA
aagggacgataagaccctataaagctttatagtgtaactcttttttactagttttactaatattaaaatattaaagaaataatactattttactggggcggtgagagtatataaaaataactgcttttattttattaaacaataataattgaaaaattaattgatccatttctaatgattataagaacaagttactttagggataacagcgtaat

SEQ ID NO: 450 - DNA, 216 bp, Lithodes confundens; identical to SEQ ID NO: 372
  source 1..216: mol_type = genomic DNA

SEQ ID NO: 451 - DNA, 215 bp, Fallosquilla fallax
  source 1..215: mol_type = genomic DNA
gagggacgataagaccctataaagctttatggtatacctcttttataaaaattgttctttattaacttattttagattgtgtattattttactggggcggtaaaagtataattaagagtaactgcttttattttgtatgacaattatagttgaaaataaattgatccattattaatgattacaaaaacaagttactttagggataacagcgtaat

SEQ ID NO: 452 - DNA, 216 bp, Echinosquilla guerinii
  source 1..216: mol_type = genomic DNA
gagggacgataagaccctataaagctttatggtagtttctttttatatagattggtaaaatttaatttattaaaagaaaaatatcattctactggggcggtaaaagtatatctttggttaactgcttttgttttgtaagtcaataatagttgaaatattaattgatccattattaatgatcataagaacaagttactttagggataacagcgtaat

SEQ ID NO: 453 - DNA, 217 bp, Coronis scolopendra
  source 1..217: mol_type = genomic DNA
gagggacgataagaccctataaagctttataatactatcttttcatattaattatctggttattaacgtactaaaagaaaagtattattttactggggcggtaaaagtatataaaagaataactgcttttgttttacaaaacaactatatttgaaaaataaattgatccattattaatgatcacaagaacaagttactttagggataacagcgtaat

SEQ ID NO: 454 - DNA, 216 bp, Chorisquilla tweediei
  source 1..216: mol_type = genomic DNA
gagggacgataagaccctataaagctttatgatattttctttttatattagttagtaaaatgtagtttattaaaagaaaaatattattttactggggcggtagaagtatatttttgattaactgcttctgttttgtaagtcaattataattgaaatatgaattgatccattattaatgatcataaggacaagttactttagggataacagcgtaat

SEQ ID NO: 455 - DNA, 215 bp, Chorisquilla hystrix
  source 1..215: mol_type = genomic DNA
gagggacgataagaccctataaagctttataattattcttttttatattagttagtagaatgtagtttattaaaaagaaatgattattttactggggcggtaaaagtatattttgatcaactgcttttgttttgtaaatcaattataattgaaatataaattgatccattattaatgatcataagaacaagttactttagggataacagcgtaat

SEQ ID NO: 456 - DNA, 216 bp, Chorisquilla excavata
  source 1..216: mol_type = genomic DNA
gagggacgataagaccctataaagctttatggtacttcttttttatattaattaggaaaatgtagtttattaaaaagaaaatactattttactggggcggtaaaagtatatttttgattaactgcttttgttttgtaagtcaataataattgaaatattaattgatccattattaatgatcacaagaacaagttactttagggataacagcgtaat

SEQ ID NO: 457 - DNA, 217 bp, Busquilla plantei
  source 1..217: mol_type = genomic DNA
gagggacgataagaccctataaagctttatgatatttctttttcttataaattttcttgttattaacttattttagagctaatatcattttactggggcggtaaaagtataataaaagattaactgcttttgttttgtataacaatcttagttggaaataaattgatccattattaatgatcataagaacaagttactttagggataacagcgtaat

SEQ ID NO: 458 - DNA, 214 bp, Alima pacifica
  source 1..214: mol_type = genomic DNA
gagggacgataagaccctataaagctttataatacacgccttctttatgaaatataaaattatcaaaatataatagaacttgtattattttactggggcggtaaaagtataataaactaactgcttttgtcctgtataacaatcttaattgaaaataaattgatccattattaatgatcacaagaacaagttactttagggataacagcgtaat

SEQ ID NO: 459 - DNA, 216 bp, Alima orientalis
  source 1..216: mol_type = genomic DNA
gagggacgataagaccctataaagctttataatacaattcttttttataaattatcgcgttattaacctatcttagaattcgtattattttactggggcggtaaaagtatattaaagactaactgcttttattttgtgtaacaattttagttggaaataaattgatccattattaatgatcacaagaacaagttactttagggataacagcgtaat

SEQ ID NO: 460 - DNA, 214 bp, Alachosquilla vicina
  source 1..214: mol_type = genomic DNA
aaatgacgataagaccctataaagctttataattttctctttctatgtaaagtataagaatattaaaacattgaaaagagtaaattattttactggggcggtaaaagtataaaaaataactgcttttattttgaataacaactattattggaatttgaattgatccattaataatgatcacaagaacaagttactttagggataacagcgtaat

SEQ ID NO: 461 - DNA, 214 bp, Gonodactylellus espinosus
  source 1..214: mol_type = genomic DNA
gagtgacgataagaccctataaagctttatggtataccttttttatacaaattacatattattaacctattaaaaaagtaatatcattttactggggcggtaaaagtatattcgcttaactgcttttattatttaaaacaataataattgaacaatccattgatccattaataatgatcataaggccaagttactttagggataacagcgtaat

SEQ ID NO: 462 - DNA, 219 bp, Gonodactylellus affinis
  source 1..219: mol_type = genomic DNA
gagggacgataagaccctataaagctttataatacatttcttttacataaattattaattattaacttgttaaagaaataatattattttactggggcggtaaaagtatattttatttgaacaactgcttttgttttaaaaaacaattataattgaacaactcattgatccattaataatgatcataaggccaagttactttagggataacagcgtaat

SEQ ID NO: 463 - DNA, 214 bp, Kempella mikado
  source 1..214: mol_type = genomic DNA
gagggacgataagaccctataaagctttataatatttttctttcttataaattatattgttattaacatattttagaatttgtattattttactggggcggtaaaagtataataaaataactgcttttattttgtataacaatcttaattggtaataaattgatccattattaatgattataagaacaagttactttagggataacagcgtaat

SEQ ID NO: 464 - DNA, 216 bp, Hemisquilla californiensis
  source 1..216: mol_type = genomic DNA
gggggacgataagaccctataaagctttatgataaggcctttttgtaaagaatttctattgtgaaattactagggggtttatatcattttactggggcggtaaaagtatataattgaataactgcttttattattgaaaacaattataattgaaatacaaaatgatccattattaatgattataaaaacaagttactttagggataacagcgtaat

SEQ ID NO: 465 - DNA, 216 bp, Haptosquilla trispinosa
  source 1..216: mol_type = genomic DNA
gagggacgataagaccctataaagctttatgatagtttctttttataataattagtaaaatgtaatttattaaaagaaatatattattttactggggcggtaaaagtatatttttgattaactgcttttgttttgcaaaacaataataattgaattataaattgatccattattaatgatcataagaacaagttactttagggataacagcgtaat

SEQ ID NO: 466 - DNA, 217 bp, Haptosquilla glyptocercus
  source 1..217: mol_type = genomic DNA
gagtgacgataagaccctataaagctttatggtagtttctttttatagtaattagtataatgtaatttattaaagggaaatatatcattttactggggcggtaaaagtatatttttgattaactgcttttgttttgtaaaacaatgataattgaattataaattgatccattattaatgattacaagaacaagttactttagggataacagcgtaat

SEQ ID NO: 467 - DNA, 214 bp, Gonodactylus platysoma
  source 1..214: mol_type = genomic DNA
gagggacgataagaccctataaagctttatgttatatattcttttatgtaatctataattattaactcattgaagaaataatatcattttactggggcggtaaaagtatatttgaataactgcttttgttataaaaaacaataataattgagtaatccattgatccattattaatgatcataagaccaagttactttagggataacagcgtaat

SEQ ID NO: 468 - DNA, 215 bp, Gonodactylaceus falcatus
  source 1..215: mol_type = genomic DNA
gagggacgataagaccctataaagctttatagtaatacctttttatttaaaatataaaatgttaacttgttaaaagagtattattattttactggggcggtaaaagtatataatgattaactgcttttgtcttactaaacaataataattgaataattatgtgatccattattaatgatcaaaagtacaagttactttagggataacagcgtaat

SEQ ID NO: 469 - DNA, 217 bp, Gonodactylus childi
  source 1..217: mol_type = genomic DNA
gagggacgataagaccctataaagctttataatataattctttgatatgaagtgttacttattaatttattaaagaaatactattattttactggggcggtaaaagtatatatttggagtaactgcttttgttattttaaacaataataattgagtgtcatattgatccactaatagtgaataaaagaacaagttactttagggataacagcgtaat

SEQ ID NO: 470 - DNA, 216 bp, Gonodactylellus annularis
  source 1..216: mol_type = genomic DNA
gagggacgataagaccctataaagctttataatacgtttctcttacacaaattataaattgttaacttgttaaagaataagtattattttactggggcggtaaaagtataccatagactaactgcttttgttattaaaaacaataataattggataatcgattgatccattaataatgatcataaggccaagttactttagggataacagcgtaat

SEQ ID NO: 471 - DNA, 213 bp, Odontodactylus scyllarus
  source 1..213: mol_type = genomic DNA
gagggacgataagaccctataaagctttatgatacaactatttcttataaaatatcttttaaatcacttaatagaatgtattattttactggggcggtaaaagtataatgtattattaactgcttttgtttttaaaaacaatcataattggaaacaaattgatccattattaatgatcacaagaacaagttactttagggataacagcgttat

SEQ ID NO: 472 - DNA, 213 bp, Odontodactylus latirostris
  source 1..213: mol_type = genomic DNA
aagggacgataagaccctataaagctttataataaaatcatcttctatggaataagtgttattaaattagctgatgaggtatattattttactggggcggtaaaagtataaacgattaactgcttttgttcttaaaaacaattataattgaaaacatattgatccattattaatgatcacaagaacaagttactttagggataacagcgttat

SEQ ID NO: 473 - DNA, 214 bp, Odontodactylus havanensis
  source 1..214: mol_type = genomic DNA
aagggacgataagaccctataaagctttatgataaagttatcctctacggaataaacattattaaactagctgataacgcatatcattttactggggcggtaaaagtataataagattaactgcttttgtttttacaaacaattataattgaaaacagattgatccattattaatgatcacaagaacaagttactttagggataacagcgttat

SEQ ID NO: 474 - DNA, 213 bp, Odontodactylus cultrifer
  source 1..213: mol_type = genomic DNA
gagggacgataagaccctataaagctttatattacatatattttatataaaatgtttattattaaattatttaatgaaatgtattattttactggggcggtaaaagtatatttgagtaactgcttttgcttttaaaaacaatcataattgaaagtaaattgatccattattaatgatcacaagaacaagttactttagggataacagcgttat

SEQ ID NO: 475 - DNA, 215 bp, Neogonodactylus oerstedii
  source 1..215: mol_type = genomic DNA
gagggacgataagaccctataaagctttatggtaatatttttgtatataaaatatttcatgtaacttattaaaaaataaacaccattttactggggcggtaaaagtatatttttgattaactgcttttgttgttgaaaacagttgtagctgaataataaattgatccattattaatgatcacaagtacaagttactttagggataacagcgtaat

SEQ ID NO: 476 - DNA, 214 bp, Neogonodactylus bredini
  source 1..214: mol_type = genomic DNA
gagggacgataagaccctataaagctttatggtaaaatttttatatataaaatatttcgtgtaacttattaaaaagtaaataccattttactggggcggtaaaagtatatcttgattaactgcttttattattaaaaacagttatagctgaataataaattgatccattactaatgttcataagttcaagttactttagggataacagcgtaat

SEQ ID NO: 477 - DNA, 216 bp, Neogonodactylus bahiahondensis
  source 1..216: mol_type = genomic DNA
gagggacgataagaccctataaagctttatggtaaaatttttgtatataaattatataatattaaattattaaaaaataaataccattttactggggcggtaaaagtatatttttgattaactgcttttgttattaaaaacagtcgtagctgaataatcagttgatccattattaatgatcataagttcaagttactttagggataacagcgtaat

SEQ ID NO: 478 - DNA, 216 bp, Lysiosquillina sulcata
  source 1..216: mol_type = genomic DNA
aagggacgataagaccctataaagctttatgattacaatcttttagtgttgattattttaatattaaagtattagaaaattatatcattttactggggcggtaaaagtataaaggaataactgcttttgttgagattaaacaacggtagttggaaaataaattgatccattaataatgatcacaagaacaagttactttagggataacagcgtaat

SEQ ID NO: 479 - DNA, 216 bp, Squilla rugosa
  source 1..216: mol_type = genomic DNA
gagggacgataagaccctataaagctttataatatttcttttttatataaattgtgttattgttaacttattttaaagattttattattttactggggcggtaaaagtatattaaagattaactgcttttgttttttataacaatcgtaattggaaataaattgatccattattaatgatcacaagaacaagttactttagggataacagcgtaat

SEQ ID NO: 480 - DNA, 214 bp, Raoulserenea hieroglyphica
  source 1..214: mol_type = genomic DNA
gagtgacgataagaccctataaagctttatagtactgtttttctttataaattaatttagtattaacatgtctaaaaatgtctactattttactggggcggtaagagtataataggttaactgctttttgtatgtacaacaatgatacttgaaatataattgatccacgattagtgattataaaaccaagttactttagggataacagcgtaat

SEQ ID NO: 481 - DNA, 214 bp, Raoulserenea oxyrhyncha
  source 1..214: mol_type = genomic DNA
gagtgacgataagaccctataaagctttatagtatgagttttttttataaattaacgtgatattaacgtacttaagttttcttactattttactggggcggtaaaagtataatggattaactgctttttctgtgtaaaacaaggatgtttgaaatataattgatccacaattagtgattataaaaccaagttactttagggataacagcgtaat

SEQ ID NO: 482 - DNA, 218 bp, Pseudosquillopsis marmorata
  source 1..218: mol_type = genomic DNA
gagggacgataagaccctataaagctttatagtgttttttttttatataaattgtgctaatttagtttataaaaaaaaaacatactattttactggggcggtaaaagtatttattattgaaaaactgcttttgtttttaaaaacaaatatgattggaaataaattgatccattattaatgattataaaaacaagttactttagggataacagcgtaat

SEQ ID NO: 483 - DNA, 214 bp, Raoulserenea ornata
  source 1..214: mol_type = genomic DNA
gagtgacgataagaccctataaagctttatagtactgtttttctttataaattaatttagtattaacatgtctaaaaatgtctactattttactggggcggtaagagtataatagattaactgctttttgtatgtacaacaatgatacttgaaatataattgatccacgattagtgattataaaaccaagttactttagggataacagcgtaat

SEQ ID NO: 484 - DNA, 214 bp, Raoulserenea komaii
  source 1..214: mol_type = genomic DNA
gagtgacgataagaccctataaagctttatagtatgagttttttttataaattaacgtgatattaacgtacttaagttttcttactattttactggggcggtaaaagtataatggattaactgctttttctgtgtaaaacaaggatgtttgaaatataattgatccacaattagtgattataaaactaagttactttagggataacagcgtaat

SEQ ID NO: 485 - DNA, 216 bp, Protosquilla folini
  source 1..216: mol_type = genomic DNA
aagggacgataagaccctataaagctttatagtggcctctttttatataaattggtaaaatatagtttattaaaagaagaatactattttactggggcggtaaaagtatatttttgattaactgcttttgttttgaatgtcaataatagttgagtaataagttgatccattattaatgatcacaagaacaagttactttagggataacagcgtaat

SEQ ID NO: 486 - DNA, 208 bp, Ibacus alticrenatus
  source 1..208: mol_type = genomic DNA
aggggacgataagaccctataaatctttatattttaaatttgttagtttaaaaaatggtttaaattttgatttaataaattaaaatattttgttggggagacaggagtataaaaagtaactgctttataatttaagttaataatatttagttaagtgatcctttaatatggattttagattaagttactttagggataacagcataat

SEQ ID NO: 487 - DNA, 213 bp, Scyllarides nodifer
  source 1..213: mol_type = genomic DNA
gagggacgataagaccctataaatctttatattttgccttataaggttaaaaattaggttcttatttaattttataaggttaaatattttgttggggagacagaggcataaaaaaagtaactgccttaaagtttaagttaaaaatgtttaagttgttaagtgatcctttaataaggaaattagaataagttactttagggataacagcgtaat

SEQ ID NO: 488 - DNA, 214 bp, Scyllarides haanii
  source 1..214: mol_type = genomic DNA
gagggacgataagaccctataaatctttatattttggcttataaggttaaaaattaggttataatttagttttataaggttgaatattttgttggggagacaggagtataaaaagaaataactgctttagaattagagttaaaggtgtttaagtttaaaagtgatcctttaataaggaatgtagattaagttactttagggataacagcgtaat

SEQ ID NO: 489 - DNA, 214 bp, Scyllarides brasiliensis
  source 1..214: mol_type = genomic DNA
gagggacgataagaccctataaatctttatattttggtttatacggttaaaagttaggttattatttaattttataagtttgaatattttgttggggagacaggggcataaaaggaaataactgccttaaagtttaagttaaaaatgtttaaattattaagtgatcctttaataaagaatatagattaagttactttagggataacagcgtaat

SEQ ID NO: 490 - DNA, 215 bp, Taku spinosocarinatus
  source 1..215: mol_type = genomic DNA
gagggacgataagaccctataaagctttatagtgtagtttttttatattaattatatattttagtttattaaaaaagtaatactattttactggggcggtaaaagtatatttttgattaactgcttttgtttttaaaaacaataataattggttaatagattgatccattattaatgatcacaagaacaagttactttagggataacagcgtaat

SEQ ID NO: 491 - DNA, 217 bp, Jasus frontalis
  source 1..217: mol_type = genomic DNA
gggggacgataagaccctataaatctttatactagttagtaataacattaaagattaattttatatttttttgttagtagttttagtattttgctggggcggcaggggtataattcaataactgctctatagttgtagttaattatgtttaaattgttggtagttgatcctttagtggagattatagattaagttactttagggataacagcgcaat

SEQ ID NO: 492 - DNA, 213 bp, Menippe mercenaria
  source 1..213: mol_type = genomic DNA
gagggacgataagaccctataaagctttatatgttatttaaatttaattgaattgatttataaattttaaatttagagtaatttattttgctggggcggcataagtataagttatattaactgcttaataatgatacaataatagttgaattgttgttaaatgatcctttttaaagatttaagaacaagttactttagggataacagcgttat

SEQ ID NO: 493 - DNA, 198 bp, Procambarus paeninsulanus
  source 1..198: mol_type = genomic DNA
aagggacgataagaccctataaaactttatatttataatatagtagttagttttatttaatagtactattttagagtatttggttggggtgacaaggataaaatataaaataactgtcttttttttttacagtaatgtttgttttaatgatcctaaaagggattaaagattaagttactttagggataacagcgtaat

SEQ ID NO: 494 - DNA, 220 bp, Puerulus sewelli
  source 1..220: mol_type = genomic DNA
gggggacgataagaccctataaatctttatatttgccgtagaaaagagtgtgtattagggctaaagatatattattttgttggggcgatattttgttggggcgacaagggtataatgagtaactgctttaaaagtttaattagataatatctagtttgagaataagagatcctttactaaggattgttaaaataagttactttagggataacagcgtaat

SEQ ID NO: 495 - DNA, 219 bp, Panulirus polyphagus
  source 1..219: mol_type = genomic DNA
gggggacgataagaccctataaatctttatgttttatctagtgaatttcagagattagttacatatttggtgttactacggtaaaatattttgttggggcgacaggagtataacaagtaactgctttggatatagaattaattatttttatagtgagaagttaaattgatcctcttctggagaaatcagactaagttactttagggataacagcgtaat

SEQ ID NO: 496 - DNA, 216 bp, Panulirus longipes
  source 1..216: mol_type = genomic DNA
gggggacgataagaccctataaatcttgatggtcaaaatattaatttttatgtagtagttataagcttaaatttaatactttgactattttgttggggcgacaggggtataattagtaactgctttgagtaaagaattaattatttttattgtgttgtgtttgagatccttctttgaagatatcagatcaagttactttagggataacagcgtaat

SEQ ID NO: 497 - DNA, 216 bp, Panulirus penicillatus; identical to SEQ ID NO: 380
  source 1..216: mol_type = genomic DNA

SEQ ID NO: 498 - DNA, 216 bp, Panulirus interruptus
  source 1..216: mol_type = genomic DNA
gggggacgataagaccctataaatctttatagctttgattataagttttagagattagttataagtctaaaattattttttgagctattttgttggggcgacaaaagtataacaagtaactgctttggttaataaattaattatttttataatgttattttaaagatccttctttgaagatgacagaataagttactttagggataacagcgtaat

SEQ ID NO: 499 - DNA, 216 bp, Panulirus marginatus
  source 1..216: mol_type = genomic DNA
gggggacgataagaccctataaatcttgataattaaagtaatagtttttacgtagtagttataagcttaaatctaatgctttgattattttgttggggcgacaagggtataataaataactgccttgaatagatagctaattatttttattatgttatgtttaagatccttctttgaagatatcagatcaagttactttagggataacagcgtaat

SEQ ID NO: 500 - DNA, 209 bp, Ibacus peronii
  source 1..209: mol_type = genomic DNA
aggggacgataagaccctataaatctttatatttaaagtttgttaatttaaaaagtagattaaaattttaatttaataaatttaaatattttgttggggagacaggagtataaaaaataactgctttaaagtttgagttaataatatttaattaagtgatcctttactatggattttagattaagttactttagggataacagcataat

SEQ ID NO: 501 - DNA, 210 bp, Ibacus chacei
  source 1..210: mol_type = genomic DNA
aggggacgataagaccctataaatctttatatttggagtttgttaatttgaaaagtaaaattaaaattttaatttaatgaatttaaatattttgttggggagacaggagtataaaaaataactgctttaaaatttgagttaataatatttaattaagtgatcctttattatggattttagattaagttactttagggataacagcataat

SEQ ID NO: 502 - DNA, 210 bp, Charybdis hellerii
  source 1..210: mol_type = genomic DNA
gagggacgataagaccctataaagctttataaatttgaagattttgtgtatttttatataaaaatgaattttgaagattttatttggttggggcgacaatggtataatgagaataactgctattaaaattaacaattatatttgaattattaataattgatcctttataaggattttaagatcaagttactttagggataacagcgttat

SEQ ID NO: 503 - DNA, 199 bp, Faxonella clypeata
  source 1..199: mol_type = genomic DNA
aagggacgataagaccctataaaactttatatttaataatagtaattaattttattttaagagtattatttgaaagtatttggttggggtgacaaggataaaatataaaataactgtcttttttttattacaataatatttggtttaatgatcctaaaatggattaaagattaagttactttagggataacagcgtaat

SEQ ID NO: 504 - DNA, 198 bp, Fallicambarus kountzeae
  source 1..198: mol_type = genomic DNA
aagggacgataagaccctataaaactttatattttataagtggtagttaattttattttgtaatactatttaggagtatttggttggggtgacaaagataaaataaaaaataactgtcttttttatttacagtaatgtttgatttaatgatccttaaaaggattaaagattaagttactttagggataacagcgtaat

SEQ ID NO: 505 - DNA, 210 bp, Arenaeus mexicanus
  source 1..210: mol_type = genomic DNA
gagggacgataagaccctataaagctttataagtgtaggtattttgtatattttatgttataataataatttgttgaatacttatttggttggggcaacaagagtataaagagagtaactgctaagatttaaaacaattatatttgattaaaaagtaattgatcccaagaggattaaaagatcaagctactttagggataacagcgtaat

SEQ ID NO: 506 - DNA, 199 bp, Cambarus tartarus
  source 1..199: mol_type = genomic DNA
aagggacgataagaccctataaaactttatatttaggagtagaagttagttttattttattggctctatttaaaagtatttggttggggtgacataaataaaaaatgaaataattgtttttattttttacaatgatatttgggtttgtgatcctgaagtggattgaaagattaagttactttagggataacagcgtaat

SEQ ID NO: 507 - DNA, 213 bp, Chionoecetes tanneri
  source 1..213: mol_type = genomic DNA
gggggacgataagaccctataaagctttacatataagtagagtttacttaattaaaaagtaaaagtttaatctaattaatgtgttttgttggggcgacataaatataatttatattaactgtttataaattaatacaataataaatgattctaatgtaattgatccttattaaagattaaaagactaagttactttagggataacagcgttat

SEQ ID NO: 508 - DNA, 210 bp, Thenus unimaculatus
  source 1..210: mol_type = genomic DNA
aggggacgataagaccctataaatctttatattaatttattgttaagctggaaaatagaattaattcgtggggtaacaattttaaatattttgttggggagacagaagtataaaagaataactactttggataaagagttaatgatatttaatgtagagaccctttcctaagggttatagaacaagttactttagggataacagcgtaat

SEQ ID NO: 509 - DNA, 210 bp, Thenus indicus
  source 1..210: mol_type = genomic DNA
gggggacgataagaccctataaatctttatattaatttgttgttaaattggaaaatagaattaatttcgggcttaacaattttaaatattttgttggggagacagaagtataaataaataactactttaaagagaaagttaatgatatttaatgtagagatcctttactaaggattgtagaacaagttactttagggataacagcgtaat

SEQ ID NO: 510 - DNA, 217 bp, Haptosquilla hamifera
  source 1..217: mol_type = genomic DNA
gagtgacgataagaccctataaagctttatagtagtgtctttttataataattagtaaaatgtaatttattgaaagaagtatactattttactggggcggtaaaagtatattttttgattaactgcttttgttttgcaagacaatgataattgaattatgaattgatccattattaatgattataagaacaagttactttagggataacagcgtaat

SEQ ID NO: 511 - DNA, 145 bp, Lithodes turritus
  source 1..145: mol_type = genomic DNA
caaaaagacgataagaccctataaatcgttacgctggggcggcgtagatatataaataaactgtctataatttagatacaataatcattgaattgatccttaaatagattaaaagattaagatactttagggataacagcgttat

SEQ ID NO: 512 - DNA, 198 bp, Bouchardina robisoni
  source 1..198: mol_type = genomic DNA
aagggacgataagaccctataaaactttatatttaagagtagtagttagttttgtttaaggagtagtatttaaaaatatttggttggggtgacaaggatagaataaaaaataactgtcttttttttttacagtaatatttggtttaatgatcctaaaaaggattaaagattaagttactttagggataacagcgtaat

SEQ ID NO: 513 - DNA, 200 bp, Troglocambarus maclanei
  source 1..200: mol_type = genomic DNA
aagggacgataagaccctataaaactttatatttataaggtagtaagttagttttatttaaaggtactatgttagaatgtttggttggggtgacaaggataaaatataaaataactgtcttttttttttacagtaatgtttggtttaatgattctaaaagggattaaaagattaagttactttagggataacagcgtaat

SEQ ID NO: 514 - DNA, 199 bp, Hobbseus yalobushensis
  source 1..199: mol_type = genomic DNA
aagggacgataagaccctataaaactttatatttgaaaatagtagttaattttgtttagtaaagtagtatttaaaagtatttggttggggtgacaaggatagaatataaaataactgtcttttttttttacaataatatttgttttgatgatcctgaaatggattaaagattaagttactttagggataacagcgtaat

SEQ ID NO: 515 - DNA, 200 bp, Hobbseus prominens
  source 1..200: mol_type = genomic DNA
aagggacgataagaccctataaaactttatatttgaaaatagtagttaattttatttaataaagtattatttaaaagtatttggttggggtgacaaggatagaatataaaataactgtctttttttttttacaataatatttgttttaatgatcctgaaatggattaaagattaagttactttagggataacagcgtaat

SEQ ID NO: 516 - DNA, 210 bp, Charybdis lucifera; identical to SEQ ID NO: 502
  source 1..210: mol_type = genomic DNA

SEQ ID NO: 517 - DNA, 199 bp, Hobbseus petilus
  source 1..199: mol_type = genomic DNA
aagggacgataagaccctataaaactttatatttgaaaatagtagttaattttatttaataaagtattatttaaaagtatttggttggggtgacaaggatagaatataaaataactgtcttttttttttacaataatatttgttttaatgatcctgaaatggattaaagattaagttactttagggataacagcgtaat

SEQ ID NO: 518 - DNA, 199 bp, Faxonella creaseri
  source 1..199: mol_type = genomic DNA
aagggacgataagaccctataaaactttatatttaataatagtaattaattttattttaagaagtattatttgaaagtatttggttggggtgacaaggataaaatataaaataactgtctttttttattacaataatatttggtttaatgatcctaaaatggattaaagattaagttactttagggataacagcgtaat

SEQ ID NO: 519 - DNA, 209 bp, Thranita danae
  source 1..209: mol_type = genomic DNA
aagggacgataagaccctataaagctttataagtttagagattttatatatttttctatagcaataatttcttgaaagcttatttggttggggcgacaatagtataattgaaataactgctttaaaataatacaaatatatttgaattataattagttgatcctttataaagatttaagatcaagttactttagggataacagcgttat

SEQ ID NO: 520 - DNA, 221 bp, Monomia petrea
  source 1..221: mol_type = genomic DNA
aggggacgataagaccctataaagctttataaatttaaagattttagagattttatacctaaatttattttttgatagtttatttggttggggcgacaaaggtataattatggataactgccattaatgtttatacatatatctttgatttaaggcttaaataataattgatcctttttaaggatttaaagatcaagttactttagggataacagcgtaat

SEQ ID NO: 521 - DNA, 216 bp, Neogonodactylus wennerae
  source 1..216: mol_type = genomic DNA
gagggacgataagaccctataaagctttatggtagaatttttatatataaattataaaatatcaacttattagaaaataaataccattatactggggcggtaaaagtatatttttgattaactgcttttgttgttaaaaacagttgtggctgaataatcagttgatccattattaatgatcataagttcaagttactttagggataacagcgtaat

SEQ ID NO: 522 - DNA, 216 bp, Xiphonectes pseudohastatoides
  source 1..216: mol_type = genomic DNA
aggggacgataagaccctataaaactttataagtttaaagatctctactatttttttttataaagttttttttgatagcttatttggttggggcgacaaaggtataattatgaataactgccattaaaattaatacatttattgatgattgttgataaatagttgatcctttttaaagattaaaagatcaagttactttagggataacagcgtaat

SEQ ID NO: 523 - DNA, 214 bp, Gonodactylellus viridis
  source 1..214: mol_type = genomic DNA
gagggacgataagaccctataaagctttataatatatttcttttatttaaattatagtttttaactcattaaagaaaatatattattttactggggcggtaaaagtatatttttgattaactgcttttgttctaacaaacaattataattgaatatccattgatccattattaatgagcataagaccaagttactttagggataacagcgtaat

SEQ ID NO: 524 - DNA, 215 bp, Gonodactylaceus ternatensis
  source 1..215: mol_type = genomic DNA
aagtgacgataagaccctataaagctttatagtagttcctttctatttagattataaaatttcaacttttttaaaggctattactattttactggggcggtaaaagtatattatgattaactgcttttgtcttgacaaacaatagtaattggataattatgtgatccattattaatgatcaaaagtacaagttactttagggataacagcgtaat

SEQ ID NO: 525 - DNA, 221 bp, Belosquilla laevis
  source 1..221: mol_type = genomic DNA
atgacgaacaaacccatactatagctttataatatgttcttcttatgtaagttatcccgcttattaaactattttaagaatcatattattttaccggggcggtaatggtataatttgtcgagattaactgcttttcttttgtatgacatctttacttgacaactaattgatccattattaatgatcacaagaacaagttactttagggataacagcgtact

SEQ ID NO: 526 - DNA, 199 bp, Procambarus okaloosae
  source 1..199: mol_type = genomic DNA
aagggacgataagaccctataaaactttatatttataatatagtagttagttttatttaatagtactattttagagtatttggttggggtgacaaggataaaatataaaataactgtctttttttttttacaataatgtctgttttaatgatcctaaaagggattaaagattaagttactttagggataacagcgtaat

SEQ ID NO: 527 - DNA, 198 bp, Procambarus morrisi
  source 1..198: mol_type = genomic DNA
aagggacgataagaccctataaaactttatatttataagatattagttagttttatttaaaagtattatattaaaatatttggttggggtgacaaggatagaatacaaaataactgtcttttttttttacagtaatgtttggtttaatgattctaaagggattaaaagattaagttactttagggataacagcgtaat

SEQ ID NO: 528 - DNA, 199 bp, Procambarus milleri
  source 1..199: mol_type = genomic DNA
aagggacgataagaccctataaaactttatatttataatatagtaattagttttatttaataatattatattaaagtatttggttggggtgacaaggatataatataaaataactgtcttttttttttacagtaatatttggtttaatgatcctagaagggattaaaagattaagttactttagggataacagcgtaat

SEQ ID NO: 529 - DNA, 198 bp, Procambarus mancus
  source 1..198: mol_type = genomic DNA
aagggacgataagaccctataaaactttatatttataaaatagtagttagttttgtttagaggtattaatttagagtatttggttggggtgacaaggataaaatataaaataactgtctttttttattacagtaatgtttggtttaatgatcctaaaagggatttaagattaagttactttagggataacagcgtaat

SEQ ID NO: 530 - DNA, 200 bp, Procambarus lunzi
  source 1..200: mol_type = genomic DNA
aagggacgataagaccctataaaactttatatttataagtagtagttagttttatttaaaaatattatattaaaatatttggttggggtgacaaggataaaatataaaataactgttctttttttttttacagtaatgtttgatttaatgatcctaaaaaggattaaaagattaagttactttagggataacagcgtaat

SEQ ID NO: 531 - DNA, 199 bp, Hobbseus cristatus
  source 1..199: mol_type = genomic DNA
aagggacgataagaccctataaaactttatatttgagaatagtagttaattttgtttagtaaagtattatttaaaagtatttggttggggtgacaaggatagaatataaaataactgtcttttttttttacaataatatttgttttaatgatcctggaatggattaaagattaagttactttagggataacagcgtaat

SEQ ID NO: 532 - DNA, 199 bp, Procambarus acutissimus
  source 1..199: mol_type = genomic DNA
aagggacgataagaccctataaaactttatatttataaggtagtagttagttttattttaaagtattattttagagtatttggttggggtgacaaggataaaatataaaataactgtctttttttttttacagtaatgtttggtttaatgatcctaaaagggattaaagattaagttactttagggataacagcgtaat

SEQ ID NO: 533 - DNA, 198 bp, Faxonius pagei
  source 1..198: mol_type = genomic DNA
aagggacgataagaccctataaaactttatatttagaagtagaagttaattttattttagtattactatttaaaatatttggttggggcgacgaggataaaaggttaaataactgtctttttttttttacaataatatttggtttaatgagcctaaaagggatttaagattaagttactttagggataacagcgtaat

SEQ ID NO: 534 - DNA, 215 bp, Manningia pilaensis
  source 1..215: mol_type = genomic DNA
gagggacgataagaccctataaagctttataataatttttttttataaaaattatggtattataacttattaaaaaatcattattattttactggggcggtaaaagtataatttagattaactgcttttaacataaaaaccaataataattgagaacaatttgatccattattaatgatcataagaacaagttactttagggataacagcgtaat

SEQ ID NO: 535 - DNA, 203 bp, Pontastacus leptodactylus
  source 1..203: mol_type = genomic DNA
aggggacgataagaccctataaaactttatattttaaaataataattaatttttatatagaagatttatttttaaatattttattggggtgataaagataaaatgtgagataactgtctttttttttttacaattaatatttgagtaaaagatcttaacaaaggaaataagagattaagttactttagggataacagcgtaat

SEQ ID NO: 536 - DNA, 199 bp, Procambarus zonangulus
  source 1..199: mol_type = genomic DNA
aagggacgataagaccctataaaactttatatttataagatagtagctagttttatttaaaagtattattttggagtatttggttggggtgacaaggataaaatataaaataactgtctttttttttttacagtaatgtttggtttaatgatcctaaaagggattaaagattaagttactttagggataacagcgtaat

SEQ ID NO: 537 - DNA, 199 bp, Procambarus seminolae
  source 1..199: mol_type = genomic DNA
aagggacgataagaccctataaaactttatatttataaggtagtagatagttttatttataagtattatattagaatatttggttggggtgacaaggataaaatataaaataactgtcttttttttttacagtaatatttggtttaatgatcctaaaatggattaaaagattaagttactttagggataacagcgtaat

SEQ ID NO: 538 - DNA, 199 bp, Procambarus seminolae; identical to SEQ ID NO: 537
  source 1..199: mol_type = genomic DNA

SEQ ID NO: 539 - DNA, 198 bp, Procambarus pycnogonopodus
  source 1..198: mol_type = genomic DNA
agggacgataagaccctataaaactttatatttctaacgtagtaattagttttatttgagagtactatattaaagtatttggttggggtgacaaggataaaatataaaataactgtcttttttttttacagtaatatttggtttaatgatccttaaaaggattagaagattaagttactttagggataacagcgtaat

SEQ ID NO: 540 - DNA, 199 bp, Procambarus orcinus
  source 1..199: mol_type = genomic DNA
gagggacgataagaccctataaaactttatatttataaggtagtaattagttttatttaaaagtattatattagaatatttggttggggtgacaaggataaaatatagaataactgtcttttttttttacagtaatatttggtttaatgggcctgaaaaggattaaaagattaagttactttagggataacagcgtaat

SEQ ID NO: 541 - DNA, 198 bp, Procambarus pallidus
  source 1..198: mol_type = genomic DNA
agggacgataagaccctataaaactttatatttataaggtagtaattagttttatttaaaagtattatattagaatatttggttggggtgacaaggataaaatacagaataactgtcttttttttttacagtaatatttggtttaatgggcctgaaaaggattaaaagattaagttactttagggataacagcgtaat

SEQ ID NO: 542 - DNA, 216 bp, Alima maxima
  source 1..216: mol_type = genomic DNA
gggggacgataagaccctataaagctttatattacgtttcttctttataaaatatccggttgttaacttattttagaaattgtattattttactggggcggtaaaagtataaataagattaactgcttttgtcttgcataacaattttaattggaaataaattgatccattaataatgatcacaagaacaagttactttagggataacagcgtaat

SEQ ID NO: 543 - DNA, 214 bp, Scyllarides deceptor
  source 1..214: mol_type = genomic DNA
gagggaygataagaccctgtaaatctttatattttggctgataaggttaaaagttaggttaktatttaattttataagtttgaatattttgttggggagacaggggyataaaaggaaataactgccttaragtttaagttaaaaatgtttaaattattaagtgatcctttaataaggaatatagactaagttactttagggataacagcgtaat

SEQ ID NO: 544 - DNA, 218 bp, Monomia argentata
  source 1..218: mol_type = genomic DNA
aggggacgataagaccctataaagctttataagtttaggaattttaaatattttgtagataaatttattttccaatatcttatttggttggggcgacaaaggtataattatgaataactgctattattaacaatacatctatcgttggctaaaatttataataattgatcctctttagagattaaaagatcaagttactttagggataacagcataat

SEQ ID NO: 545 - DNA, 212 bp, Xiphonectes pulchricristatus
  source 1..212: mol_type = genomic DNA
gagggacgataagaccctataaagctttatatattaaatgattttatggatttaaaaaataaaaattattctgaaaaatttatttggttggggcgacagaggtataatttagataactgctaggaatttatacaattattcttgaattatgtttgtaaatgatccttgttagagattaaaagatcaagttactttagggataacagcgtaat

SEQ ID NO: 546 - DNA, 214 bp, Paralithodes platypus
  source 1..214: mol_type = genomic DNA
aaaagacgataagaccctataaatctttacaataaatatattttatattttagcttataagtaaataagaaataaaaatataggtttgttacgctggggcggcgtagatatataaataaactgtctatagtttaaatacaataattattgcttaatacaaattgatccttaaatagattaaaagattaagatactttagggataacagcgttat

SEQ ID NO: 547 - DNA, 212 bp, Lopholithodes foraminatus
  source 1..212: mol_type = genomic DNA
aagacgataagaccctataaatctttacaataaatatattttatattttagcttataagtgtataagaaataaaaatatagatttgttgcgctggggcggcgtagatatataaataaactgtctataatttaaatacaataatcattgtttaatacaaattgatccttaaatagattaaaagattaagatactttagggataacagcgttat

SEQ ID NO: 548 - DNA, 214 bp, Faughnia formosae
  source 1..214: mol_type = genomic DNA
gagggacgataagaccctataaagctttatgattcctttttttattataaattatatattttagtttactaaaaaacaggtattattttactggggcggtaaaagtatgtttttgggtaactgcttttatttttaaaaacaattatgtttggaaataaaatggtccattattaatgattataagaacaagttactttagggataacagcgtaat

SEQ ID NO: 549 - DNA, 216 bp, Faughnia profunda
  source 1..216: mol_type = genomic DNA
gggggacgataagaccctataaagctttatggtagtttcttttaatatgaattgtatgatttctagtttattgaagaaaaattactattttactggggcggtaaaagtatgctttcggttaactgcttttgtttttaaaaacaaacatggttggaaataaattgatccattattaatgattataagaacaagttactttagggataacagcgtaat

SEQ ID NO: 550 - DNA, 221 bp, Bathysquilla crassispinosa
  source 1..221: mol_type = genomic DNA
gagggacgataagaccctataaagctttatggtgtactactttgttacaaaatataataattaaaacttattgaagtagtgggcattattttactggggcggtaaaagtataaataatctgagtaactgcttttattgtgctaaacaattataattgacttttaaattgatccattactaatgatcacaagaacaagttactttagggataacagcgtaat

SEQ ID NO: 551 - DNA, 218 bp, Eriocheir sinensis
  source 1..218: mol_type = genomic DNA
taaagggacgataagaccctataaagcttaatattaaattttatatagtcaaattttaggttataaagattttgtaaattgaatttattttattggggcgataagagtaaaatgattattaactgcttaacattaagtacacttatgtgtgattaaaattttaaatgatcctaattaaagattaaaagtttaagttactttagggataacagcgttat

SEQ ID NO: 552 - DNA, 216 bp, Harpiosquilla harpax
  source 1..216: mol_type = genomic DNA
gagggacgataagaccctataaagctttatgacatatttttttttataaattattttgttattaacttaatataaaatgtatgtcattttactggggcggtaaaagtataattaaagaataactgcttttgtttttaataacaattatagttggaaatgaattgatccattattaatgatcacaagaacaagttactttagggataacagcgtaat

SEQ ID NO: 553 - DNA, 215 bp, Callinectes sapidus
  source 1..215: mol_type = genomic DNA
ccatagggacgataagaccctataaagctttataaatttattaatttcgtttattttttattataataatgatgataagagaatttatttggttggggcaaccgaagtataattttaaataactgctataatttaatacaatgatttttgtcttatacaataattgatcctcaaagagattaaagaccaagttactttagggataacagcgtaat

SEQ ID NO: 554 - DNA, 216 bp, Squilla mantis
  source 1..216: mol_type = genomic DNA
gagggacgataagaccctataaagctttataatacttcttttttatataaattgttctattattaacttattttaaagattgtattattttactggggcggtaaaagtatattaaagactaactgcttttattttttataacaattatgattgaatgtaaattgatccattattaatgatcacaagaacaagttactttagggataacagcgtaat

SEQ ID NO: 555 - DNA, 216 bp, Portunus trituberculatus
  source 1..216: mol_type = genomic DNA
gagggacgataagaccctataaagctttataaatttagaaattttatatatttttttaataatgataatcttttgaaaatttattgggttggggcaacaaaagtataattattaataactgctagaaagtattacaattgtttttgtcataaagaaaattagttgatccctgaaagggattaaaagatcaagttactttagggataacagcgtaat

SEQ ID NO: 556 - DNA, 216 bp, Panulirus japonicus
  source 1..216: mol_type = genomic DNA
gggggacgataagaccctataaatcttgatgattaagataataaattttaggcagtagtaataagtttaaatttaatgtcttaattattttgttggggcgacaggagtataagaagtaactgctttgaaaaaataattaattatttttatgatgttttgtttgtgatccttctatgaagataatagatcaagttactttagggataacagcgtaat

SEQ ID NO: 557 - DNA, 218 bp, Cancer pagurus
  source 1..218: mol_type = genomic DNA
aggggacgataagaccctataaagctttatattttagctataatttaattaaattaattagatcaaaggttttaataagttattatattttattggggaggtataagtataatttatgtaactgcttataagttagtacactgataattgattagagtttcaatgatcctttaagataaagatttaagactaagttactttagggataacagcgttat

SEQ ID NO: 558 - DNA, 213 bp, Chionoecetes japonicus
  source 1..213: mol_type = genomic DNA
gggggacgataagaccctataaagctttacatataagtagagtttacttaattaaaaagtaaaagtttaatctaattaatgtgttttgttggggcgacataaatataatttatattaactgtttataatttaatacaataataaatgattttaatgtagttgatccttattaaagattaaaagactaagttactttagggataacagcgttat

SEQ ID NO: 559 - DNA, 215 bp, Scylla tranquebarica
  source 1..215: mol_type = genomic DNA
gagggacgataagaccctataaagctttataaattaagtaagttagaacaaattttatagaataaaggtaaatttaatatatttatttggttggggcgacaatggtataaattaaataactgctattaaaattaaacaattatatttgattaaaattataattgatccttattatggattaaaagatcaagttactttagggataacagcgtaat

SEQ ID NO: 560 - DNA, 216 bp, Scylla serrata
  source 1..216: mol_type = genomic DNA
ctagagggacgataagaccctataaagcttcataagttaaataaattagatggatttttagaataaaagtaaatttagtaagcttatttagttggggcgacaagggtataaaattaaataactgctattaaggttagacaattatatttggttggattgtaattgatcctcgctatggattaaaagattaagttactttagggataacagcgtaat

SEQ ID NO: 561 - DNA, 216 bp, Eriocheir hepuensis
  source 1..216: mol_type = genomic DNA
aagggacgataagaccctataaagcttaatattaaattttatatagtcaaattttaaattataaagattttgtaaattgaatttattttattggggcgataagagtaaaatgattattaactgcttaacattaagtacacttatatgtgattaaaattttaaatgatcctaattaaagattaaaagtttaagttactttagggataacagcgttat

SEQ ID NO: 562 - DNA, 216 bp, Eriocheir japonica
  source 1..216: mol_type = genomic DNA
aagggacgataagaccctataaagcttaatattaaattttatttagtcaaattttaggttataaagattttgtaaattgaatttattttattggggcgataagagtaaaatgattattaactgcttaatattaagtacacttatatgtgattaaaattttaaatgatcctaattaaagattaaaagtttaagttactttagggataacagcgttat

SEQ ID NO: 563 - DNA, 205 bp, Cherax destructor
  source 1..205: mol_type = genomic DNA
gggggacgataagaccctataaagtttgacattaaattaattaagggtaatttagattataaagtcttattattatataagtgtttagttggggcgactaggatataagttatttaactgtttcttcattcgaatcaaaaatttttgattttatgatcctttttaagggtactagagtaaattactttagggataacagcgtaat

SEQ ID NO: 564 - DNA, 215 bp, Squilla empusa
  source 1..215: mol_type = genomic DNA
gagggacgataagaccctataaagctttatgatatttcttttttgtataaaatatttattgttaacatatttaaaagtttatattattttactggggcggtaaaagtatattaaagactaactgcttttgttttttataacaattataattgaaaataaattgatccattattaatgatcataagaacaagttactttagggataacagcgtaat

SEQ ID NO: 565 - DNA, 216 bp, Lysiosquillina maculata
  source 1..216: mol_type = genomic DNA
aagggacgataagaccctataaagctttatgatgtatattcgctaatgttgattttcgtaatattaaagcgttagagaatcatatcattttactggggcggtaaaagtataaaagaataactgcttttgttgaaattaaacaatgatagttggagaataaattgatccattaataatgatcataagaacaagttactttagggataacagcgtaat

SEQ ID NO: 566 - DNA, 216 bp, Gonodactylus chiragra
  source 1..216: mol_type = genomic DNA
gagggacgataagaccctataaagctttataatggattcttgttatataaattatcaattgttaacatgttaaaagaataatattattttactggggcggtaaaagtatatatctgagtaactgcttttgttttttaaaacagtgatgactgacttattaattgatccactaatagtgagcataaggacaagttactttagggataacagcgtaat

SEQ ID NO: 567 - DNA, 217 bp, Panulirus homarus
  source 1..217: mol_type = genomic DNA
gggggacgataagaccctataaatctttatgttttaactaataaattttagagattagttaaatatttagtgttattaagttaaatattttgttggggcgacaggagtacaataagtaactgctttgtttataaagttaattatttttattatggaaattgttattgatcctttttggagaaatcagaataagttactttagggataacagcgtaat

SEQ ID NO: 568 - DNA, 210 bp, Homarus americanus
  source 1..210: mol_type = genomic DNA
taaagggacgataagaccctataaagcttaataatttagtatataattagatgagttgaaagtttaatattatttatatactaaattatttcgttggggcgacgatgatataatttgtaactgtttaaatttaaaatacagagatatttgtgtgtaatgatccttgttgttgattaaaaatttaagttactttagggataacagcgttat

SEQ ID NO: 569 - DNA, 218 bp, Panulirus ornatus
  source 1..218: mol_type = genomic DNA
gggggacgataagaccctataaatctttatactttaactaataaattttaaagattagttgcaaatttagtgttattaagttaaaatattttgttggggcgacaggagtataataagtaactgctttgtttataaatttaattatttttaatgtgtaaattgtattgatcctctgttggagaaatcagactaagttactttagggataacagcgtaat

SEQ ID NO: 570 - DNA, 217 bp, Oratosquilla oratoria
  source 1..217: mol_type = genomic DNA
gagggacgataagaccctataaagctttatgatactattcttttttataaattgttttgttattaacttattttagaattagtattattttactggggcggtaaaagtataataaaagaataactgcttttgttttgtataacaattttagttggaaataaattgatccattattaatgatcataagaacaagttactttagggataacagcgtaat

SEQ ID NO: 571 - DNA, 217 bp, Panulirus stimpsoni
  source 1..217: mol_type = genomic DNA
gggggacgataagaccctataaatctttatattttatttaataaattttaaaaattagttatacatttagcgatattaagtcaaatattttgttggggcgacaggagtataataagtaactgctttgtttgtaaagttaattatttttattgtgtagagttaaattgatcctttctagagaaatcagatcaagttactttagggataacagcgtaat

SEQ ID NO: 572 - DNA, 211 bp, Charybdis japonica
  source 1..211: mol_type = genomic DNA
gagggacgataagaccctataaagctttataaatttaaagattttatatatttttatataaaaataaattccagaggatttattttgttggggcgacaatggtataatgaaaataactgctattaaaattaacaattatatttgtttatgtaaataattgatcctttataaggattttaagatcaagttactttagggataacagcgttat

SEQ ID NO: 573 - DNA, 214 bp, Scylla paramamosain
  source 1..214: mol_type = genomic DNA
gagggacgataagaccctataaagctttataagttaagtaagttaaattaattttttaaaataaaaataagcttaaaaaatttatttggttggggcgacaatggtataaattaaataactgctattaaaattaagcaattatatttgattagaattataattgatccttattatggattaaaagatcaagttactttagggataacagcgtaat

SEQ ID NO: 574 - DNA, 214 bp, Scylla olivacea
  source 1..214: mol_type = genomic DNA
gagggacgataagaccctataaagctttatagattgagtggattaaaccaattttttagaataaaagtaaatttggaagatttatttggttggggcgacaaatgtataatgttaaataactgctattaaatttaaacaattatatttgataaaaatttaaatgatccttaataaagattaaaagatcaagttactttagggataacagcgtaat

SEQ ID NO: 575 - DNA, 205 bp, Cherax quadricarinatus
  source 1..205: mol_type = genomic DNA
gagggacgataagaccctataaagtttatacatgaagttggttaagagtgatttaaggtgttaaagtttattatcagcagggtgtttagttggggcgactaggatataaattatataactgttttttgtttaaatcagagatatttgttcatatgatccctttttaggattagagaataaattactttagggataacagcgtaat

SEQ ID NO: 576 - DNA, 206 bp, Cherax cainii
  source 1..206: mol_type = genomic DNA
aagggacgataagaccctataaagtttatacatccaaatgattgaaaataattaagaaaattaaagtttattattagggagatgtttagttggggcgactaggatataagttaatttaactgtcctaaattaaaatcaaagatatttgagtttgtgatccttttttaggattaaagaacaagttactttagggataacagcgtaat

SEQ ID NO: 577 - DNA, 216 bp, Paralithodes camtschaticus
  source 1..216: mol_type = genomic DNA
caaaaagacgataagaccctataaatctttacaataaatatattttatattttagtttataagtgaatgagatataaaaatataggtttgttgcgctggggcggcgtagatatataaataaactgtctatagtttaaatacaataatcattgcttaatataaattgatccttaaatagattaaaagattaagatactttagggataacagcgttat

SEQ ID NO: 578 - DNA, 216 bp, Paralithodes camtschaticus; identical to SEQ ID NO: 577
  source 1..216: mol_type = genomic DNA

SEQ ID NO: 579 - DNA, 215 bp, Scyllarides latus
  source 1..215: mol_type = genomic DNA
gagggacgataagaccctataaatctttatattttgggttataaggttaaaaattaggttatcttttaattttataaggttaaatattttgttggggagacagaggtataaaaagagataactgccttaaaatttatagttaagaatagttaagattgaaagtgatcctttaataaggaaaatagaagaagttactttagggataacagcgtaat

SEQ ID NO: 580 - DNA, 199 bp, Procambarus clarkii
  source 1..199: mol_type = genomic DNA
ggagggacgataagaccctataaaactttatatttataatatagtagttagttttatttaagggtattattttagagtatttggttggggtgacaaggataaaatattaaataactgtcttttttttttacagtgatgtttggtttaatgatcctaaaagggattaaagattaagttactttagggataacagcgtaat

SEQ ID NO: 581 - DNA, 199 bp, Procambarus fallax
  source 1..199: mol_type = genomic DNA
aagggacgataagaccctataaaactttatatttataaagtagtagttagttttatttaaaagtattatattagaatatttggttggggtgacaaggataaaatatagaataactgtcttttttttttacagcaatatttggtttaatgatcctaaaaaggattaaaagattaagttactttagggataacagcgtgat

SEQ ID NO: 582 - DNA, 211 bp, Thenus orientalis
  source 1..211: mol_type = genomic DNA
aggggacgataagaccctataaatctttatattaatttgttgttaaattggaaagtagaattaattccgggtttaataattttgaatattttgttggggagacagaagtgtaaataaataactactttaaaaaaaaaagttaatgatatttaatgaggagatcctttactaaggattgtagaataagttactttagggataacagcgtaat

SEQ ID NO: 583 - DNA, 216 bp, Lithodes nintokuae
  source 1..216: mol_type = genomic DNA
caaaaagacgataagaccctataaatctttacaataaatatattttatattttagcttataagtgtataagaaataaaaatatagatttgttgcgctggggcggcgtagatatataaataaactgtctatagtttaaatacaataatcattgcttaatacaaattgatccttaaatagattaaaagattaagatactttagggataacagcgttat

SEQ ID NO: 584 - DNA, 206 bp, Cherax cairnsensis
  source 1..206: mol_type = genomic DNA
gggggacgataagaccctataaagtttgtacactaaactagtttagagtgatttaggtttataaagtttactattggggaggtgtttagttggggcgactaggatataagttatctaactgtttctttgttgtaatcagaaatatttgagtttatgatccttttttaggattatagagtaaattactttagggataacagcgtaat

SEQ ID NO: 585 - DNA, 206 bp, Cherax cairnsensis; identical to SEQ ID NO: 584
  source 1..206: mol_type = genomic DNA

SEQ ID NO: 586 - DNA, 206 bp, Cherax dispar
  source 1..206: mol_type = genomic DNA
gggggacgataagaccctataaagtttgtacattaaactaattaaaggtggtttaggtttataaaatttgccattagggaagtgtttagttggggcgattgggatataagttatctaactgtttctttgttttaatcaaaaatatttgagtttatgatccttttttaggattaaagagtaaattactttagggataacagcgtaat

SEQ ID NO: 587 - DNA, 207 bp, Cherax quinquecarinatus
  source 1..207: mol_type = genomic DNA
aagggacgataagaccctgtaaagtttatacattaagttaattaaaaataattaaggagattaaaatttattattgggtagatgtttagttggggcgactaggatataatttaattataactgtcttagattaaggtcaaagatatttgagtttgtgatcctttcttaggattcaagaacaagttacttcagggataacagcgtaat

SEQ ID NO: 588 - DNA, 207 bp, Cherax robustus
  source 1..207: mol_type = genomic DNA
gagggacgataagaccctataaagttttgtacattaaaccaattaataggcgatttaggtttgtaaggtttactattaggtagatgtttggttggggcgactaggatataagttatttaactgttctttgttagaatcaaaggtatttgagtttatgatcctttttaagggttatagaataaattactttagggataacagcgtaat

SEQ ID NO: 589 - DNA, 206 bp, Cherax glaber
  source 1..206: mol_type = genomic DNA
aagggacgataagaccctataaagtttatatacagaactaattaaagataattaggaagaataaaatttattattaggtaggtatttagttggggcgactaggatataaattaatttaactgtttttaattaaggtcaaagatatttgagttcgtgatccttttttaggattaaagaataagttactttagggataacagcgtaat

SEQ ID NO: 590 - DNA, 206 bp, Cherax glaber; identical to SEQ ID NO: 589
  source 1..206: mol_type = genomic DNA

SEQ ID NO: 591 - DNA, 205 bp, Cherax holthuisi
  source 1..205: mol_type = genomic DNA
gagggacgataagaccctataaagtttgaacaccgggtttgttaagagtgatttagaatagtaaagtttgttactagtaaagtgtttggttggggcgactagaatataagttatataactgttttttatttgaatcagagatatctgtttatatgatccctttttaggattaaagagtaaattactttagggataacagcgtaat

SEQ ID NO: 592 - DNA, 205 bp, Astacopsis gouldi
  source 1..205: mol_type = genomic DNA
gggggacgataagaccctataaagtttaacataatgaggataaaaaattaattagattataaagtttattatcataaataatgttttgttggggcgacaagaataaaaataatttaactgttcttttttagatacaaaaatatttgtgtagggtgatcttttttagaagtattagagtaaattactttagggataacagcgtaat

SEQ ID NO: 593 - DNA, 215 bp, Portunus pelagicus
  source 1..215: mol_type = genomic DNA
gagggacgataagaccctataaagctttataaatttaaagattttatatattttttattataatgataatttctcaaaaatttattgggttggggcaacaaaagtataattatcaataactgctataaattactacaattatttttgttattaaagagttgttgatcctcgaaagggattaaaagatcaagttactttagggataacagcgtaat

SEQ ID NO: 594 - DNA, 204 bp, Paranephrops planifrons
  source 1..204: mol_type = genomic DNA
aagggacgataagaccctataaagctttacattgaatttattaaaaagtaatttaaataataaaagtttattagtagggagatgttttgttggggtgacaagaatataataaatataactgttcttttttatttcaaaaatatttgaatgggagatccttaataaagatgttagagtaagttactttagggataacagcgtaat

SEQ ID NO: 595 - DNA, 210 bp, Nephrops norvegicus
  source 1..210: mol_type = genomic DNA
taaagggacgataagaccctataaagcttaataatttaatatataaccagataaattaaaagtttaatattctttatatattaaattatttcgttggggcgacgatgatataatttgtaactgtttaaattttaaatacagagatatttgtgtgtaatgatcctttttattgattaaaaatttaagttactttagggataacagcgttat

SEQ ID NO: 596 - DNA, 212 bp, Charybdis feriata
  source 1..212: mol_type = genomic DNA
gagggacgataagaccctataaagctttataaatttaaaaatcttatatatttttatataaaaataatttttgaggagtttatttggttggggcgacagtggtataagtaatgtaactgctatagtgtttaacaaaaatatttgtttagtgtaagtaattgatcctttataaggattttaagatcaagttactttagggataacagcgttat

SEQ ID NO: 597 - DNA, 212 bp, Charybdis feriata; identical to SEQ ID NO: 596
  source 1..212: mol_type = genomic DNA

SEQ ID NO: 598 - DNA, 209 bp, Metanephrops sibogae
  source 1..209: mol_type = genomic DNA
gagggacgataagaccctataaagcttgataattttgtatataaataaataagttgttagtattatactgtctacatataaaattatttcgttggggcgacgataatataatctgtaactgtttaggttttaactcaaatatatttgtgtttaatgatccattaattgttgattaaaaatttaagttactttagggataacagcgttat

SEQ ID NO: 599 - DNA, 216 bp, Panulirus cygnus
  source 1..216: mol_type = genomic DNA
gggggacgataagaccctataaatcttgatgatcggggtagtaatttttatgtagtagttataaacttaaatttaataccttgattattttgttggggcgacaggggtataattagtaactgccttggataaacagctaattatttttattgtgttatgtctaagatccttctttgaagatatcagatcaagttactttagggataacagcgtaat

SEQ ID NO: 600 - DNA, 211 bp, Metanephrops thomsoni
  source 1..211: mol_type = genomic DNA
gagggacgataagaccctataaagcttgataattttatatataaataaataagttgttagtgttatatcatctatttataaaattatttcgttggggcgacgataatataatttgtaactgtttaagatttagattcaaatatatttgtgttttaatgatccattagttgttgattaaaaatttaagttactttagggataacagcgttat

SEQ ID NO: 601 - DNA, 198 bp, Faxonius limosus
  source 1..198: mol_type = genomic DNA
ttgggacgataagaccctataaaactttatattttaaggtaggagttaatttcattttaaggttactattttaaaatgttttgttggggggacaaggatataagaaatgataactgtcttttatttttacaataatatttgatttattgatcctgaaagggatttaagattaagttactttagggataacagcgtaat

SEQ ID NO: 602 - DNA, 216 bp, Squilloides leptosquilla
  source 1..216: mol_type = genomic DNA
gagggacgataagaccctataaagctttatattatatttctttaatataaattgtaatgttattaacttattttagaaaatatattattttactggggcggtaaaagtatattaaagattaactgcttttgttttgtataacaattatagttgaaaataaaatgatccattattaatgatcataaaaccaagttactttaggggtaacagcgtaat

SEQ ID NO: 603 - DNA, 205 bp, Cherax bicarinatus
  source 1..205: mol_type = genomic DNA
gagggacgataagaccctataaagtttatacatggggctggttgagagtgattcagaatagtaaagtttattattagcaagatgtttagttggggcgactaagatataaattatataactgtcttttgtttaaatcagaggtatctgttcgtatgatccttttttaggattggagagtaaattactttagggataacagcgtaat

SEQ ID NO: 604 - DNA, 201 bp, Austropotamobius pallipes
  source 1..201: mol_type = genomic DNA
gggggacgataagaccctataaaactttatattttaaaaatattaactagttttgtataagagggtttttttttgagtattttattggggtgatagggatataataaaagataactgtctctttttttttacagagatatttgagtaaatgatcctaataagggaaagaaggttaagttactttagggataacagcgtaat

SEQ ID NO: 605 - DNA, 207 bp, Cherax tenuimanus
  source 1..207: mol_type = genomic DNA
aagggacgataagaccctataaagtttatacattcaaataattgaaaataattaagaaaaattaaagtttattattaggaagatgtttagttggggcgactaggatataagttaatttaactgtcttaaattaaagtcaaagatatttgagtttgtgatccttttttaggattcaagaacaagttactttagggataacagcgtaat

SEQ ID NO: 606 - DNA, 206 bp, Cherax boesemani
  source 1..206: mol_type = genomic DNA
gagggacgataagaccctataaagtttatacatggagttagttaagagtgatttaggatagtaaagtttgttattagcaagatgtttggttggggcgactagaatataagttatataactgttttttatttaagtcagaggtatttgtttatagtgatccctttttaggattaaagagtaaattactttagggataacagcgtaat

SEQ ID NO: 607 - DNA, 213 bp, Charybdis (Charybdis) natator
  source 1..213: mol_type = genomic DNA
gagggacgataagaccctataaagctttataaatttaaagattttgtatgtttttatataaagataaattctaaagaatttattgggttggggcgacaatggtataatgaaaataactgctattaaaattaacaactatatttggattaagttaaataattgatcctttataaggattttaagatcaagttactttagggataacagcgttat

SEQ ID NO: 608 - DNA, 198 bp, Procambarus acutus
  source 1..198: mol_type = genomic DNA
aagggacgataagaccctataaaactttatatttataagatagtagctagttttatttaaaagtattattttggagtatttggttggggtgacaaggataaaatataaaataactgtcttttttttttacagtaatgtttggtttaatgatcctaaaagggattaaagattaagttactttagggataacagcgtaat

SEQ ID NO: 609 - DNA, 199 bp, Pacifastacus leniusculus
  source 1..199: mol_type = genomic DNA
gagtgacgataagaccctataaaactttatattttaagataatagttagttttatttaagagttttattttgaaatattttattggggtgataaagatataaattaaaataactgtcttttttttttacaataatctttgaattaatgatcctaagaaaggagtaaaagatcaagttactttagggataacagcgtaat

SEQ ID NO: 610 - DNA, 208 bp, Munida gregaria
  source 1..208: mol_type = genomic DNA
aagggacgataagaccctataaatctttatattaaattatttttagttaattaataatgtaattttaacagtaaaataaaatatattgtgttggggtgatgagaatataaaattaactgttctaaattgaaaacaaatttatttgaattaataatagatccttaatagagattaaaagtttaagttactttagggataacagcgttat

SEQ ID NO: 611 - DNA, 220 bp, Panulirus versicolor
  source 1..220: mol_type = genomic DNA
gggggacgataagaccctataaatctttatgctttaattaataaatcttagaaattagttacaaatctagcgtcattgagttaaagtattttgttggggcgacagaagcataataagtaactgctttcgtttaaaaaaattaattatttttattgtgatagttatattgatcctcttatggagaaaccagatcaagttactttagggataacagcgtaat

SEQ ID NO: 612 - DNA, 198 bp, Faxonius rusticus
  source 1..198: mol_type = genomic DNA
atgggacgataagaccctataaaactttatattttaatgtagaagttaattttattttaagttcactattttaaaattttttgttggggcgacaaggatataaaaaatgataactatcttttatttttacaataatatttgatttattgatcctaaaagggattaaagattaagttactttagggataacagcgtaat

SEQ ID NO: 613 - DNA, 214 bp, Portunus sanguinolentus
  source 1..214: mol_type = genomic DNA
gagggacgataagaccctataaagctttataaatttaaagattttacatattttatctataatggtaatctctggagaatttattgggttggggcaacaaaagtataattatcaataactgctataaaataatacaattatctttgtgtgggtagattagttgatccctgaaaaggattaaaagatcaagttactttagggataacagcgtaat

SEQ ID NO: 614 - DNA, 199 bp, Procambarus alleni
  source 1..199: mol_type = genomic DNA
aagggacgataagaccctataaaactttatatttataatatagtagttagttttatttaataatattatattaaagtatttggttggggtgacaaggatataatataaaataactgtcttttttttttacagtaatatttggtttaatgatcctagaagggattaaaagattaagttactttagggataacagcgtaat

SEQ ID NO: 615 - DNA, 221 bp, Metacarcinus magister
  source 1..221: mol_type = genomic DNA
gggggacgataagaccctataaagctttatattatcagctatgatttaattaaattaaaaagaaattaaaggttttaataagctattatattttattggggagatataagtataattttatgtaactgcttataagtcgatacattgataattggtttaaattttaatgatcctttgaataaagattcaagaccaagttactttagggataacagcgttat

SEQ ID NO: 616 - DNA, 219 bp, Puerulus angulatus
  source 1..219: mol_type = genomic DNA
gggggacgataagaccctataaatctttatatttgctcagccaaaaaatgtgtattagggccaaaaatatattttttggtggggtgatattttgttggggcgacaggagtataataagtaactgctttaaaagtttaattagatatatttagtttgagtgttaaaagatcctttagtaaggattattaaaataagttactttagggataacagcgtaat

SEQ ID NO: 617 - DNA, 213 bp, Lupocycloporus gracilimanus
  source 1..213: mol_type = genomic DNA
gagggacgataagaccctataaagctttataaattcgataattttgcaaatttgtacaaataaaagtaattttgaaaaatttatttggttggggcgacaaaagtataattaaaataactgctagaatttcaaacaataatttttgatttaggtttgtaaatgatcccttataaggattaaaagatcaagttactttagggataacagcgtaat

SEQ ID NO: 618 - DNA, 223 bp, Monomia gladiator
  source 1..223: mol_type = genomic DNA
taaggggacgataagaccctataaagctttataaattcagagattttagatattttatatataaatttattttttgataatttatttggttggggcgacaagggtataattataaataactgccattaatgtttatacatatatttttgatttaaatttaatatagtagttgatcctttttaaggatttaaagatcaagttactttagggataacagcgtaat

SEQ ID NO: 619 - DNA, 218 bp, Varuna yui
  source 1..218: mol_type = genomic DNA
ttaaagtgacgataagaccctataaagctttatatagatatattatttaattaaatttttaataaaaatttagtagtaaaatttattttattggggcgataagagtaaaatttttattaactgctttaagttttgaatacatttatgtttgattaaaattttaaatgatcctaaataaagattaaaagtttaagttactttagggataacagcgttat

SEQ ID NO: 620 - DNA, 218 bp, Panulirus argus
  source 1..218: mol_type = genomic DNA
gggggacgataagaccctataaatctttacaattagggctttagttattagaaagtagttatggatctaaatctaaggtttaaattgttttgttggggcgacagaagtataataagtaactgctttggttttcatattaattgtttttattatggttagtttaaaagatcctcttttggagatcacagactaagttactttagggataacagcgtaat

SEQ ID NO: 621 - DNA, 209 bp, Munida isos
  source 1..209: mol_type = genomic DNA
aagggacgataagaccctatgaatctttatattatgttacttttaattaattaatgttgtagatatagtgataaggtggggtatattgtgttggggtgatgagaatataagaaataactgttctaaatttaaaacaaaattgtttgaggtaataatagatcctttattgagattaaaagtttaagttactttagggataacagcgttat

SEQ ID NO: 622 - DNA, 214 bp, Scyllarides squammosus
  source 1..214: mol_type = genomic DNA
gagggacgataagaccctataaatctttatacttaggtttatacggttgaaggttaggttactatttaattttataaagttaagtattttgttggggtgacaggggcataagagtgaataactgccttagagttagagttaaaagtatttaatttattaagtgatcctttagtagagaatatagactaagttactttagggataacagcgtaat

SEQ ID NO: 623 - DNA, 202 bp, Cambaroides similis
  source 1..202: mol_type = genomic DNA
gggggacgataagaccctataaaactttatatacttataatttaataataacttttgggtttaaaaatttagattagagtattttattggggcgataaagataaaattttagataactgttttattttttaacaaaaatgtttgagttttagatccttaaagagattaaaagattaagttactttagggataacagcgttat

SEQ ID NO: 624 - DNA, 200 bp, Cambarus robustus
  source 1..200: mol_type = genomic DNA
aagggacgataagaccctataaaactttatatttagaaataatgggtaagttttgtttagaagttattattttagagtatttggttggggcgacaaggataaaaagaagataactgttcttttattttacaataatgtttgggtataatgatcctaatatgggttaaaagattaagttactttagggataacagcgtaat

SEQ ID NO: 625 - DNA, 200 bp, Cambarus robustus; identical to SEQ ID NO: 624
  source 1..200: mol_type = genomic DNA

SEQ ID NO: 626 - DNA, 213 bp, Thalamita sima
  source 1..213: mol_type = genomic DNA
gagggacgataagaccctataaagctttataaacttataaattttatatgtttttttataaaaataatctttcgaaggtttatttagttggggcgacaatgatataaataatataactgtcataaaatataacaaatatatttgtttaataaggaataattgatcctttataaggattttaagatcaagttactttagggataacagcgttat

SEQ ID NO: 627 - DNA, 210 bp, Thranita crenata
  source 1..210: mol_type = genomic DNA
aagggacgataagaccctataaagctttataattttaaagattttatatatttttctatagtaataatttcttgaaaacttatttggttggggcgacaatagtataattagaataactgcttaaaattaatacaaatatatctgagttaaaattgactgatcctttataaagattttaagatcaagttactttagggataacagcgttat

SEQ ID NO: 628 - DNA, 199 bp, Orconectes luteus
  source 1..199: mol_type = genomic DNA
aggggacgataagaccctataaaactttatattttaagatggagataaattttattttaaggttactattttaaattgttttgttggggcgacaaggatataaggaaaggataactgtcttttgttcttacaataatatttgaattattgatcctaaaagggattaaagattaagttactttagggataacagcgtaat

SEQ ID NO: 629 - DNA, 198 bp, Orconectes punctimanus
  source 1..198: mol_type = genomic DNA
aagggaagataagaccctataaaactttatattttaaggtagaaattaattttatttttagattactattttaaaatattttgttggggtgacaaggatataataaaaggtaactgtcttttggtttaacaataatatttgggtaattgatcctaaaatggataaaagattaagttactttagggataacagcgtaat

SEQ ID NO: 630 - DNA, 198 bp, Orconectes sanbornii
  source 1..198: mol_type = genomic DNA
ttgggacgataagaccctataaaactttatattttaaggtagaaattaattttattttaaggttactattttaaaatgttttgttggggtgacaaggatacaagaaatgataactgtcttttatttttacaataatatttgatttattgatcctgaaagggattaaagattaagttactttagggataacagcgtaat

SEQ ID NO: 631 - DNA, 206 bp, Cherax crassimanus
  source 1..206: mol_type = genomic DNA
aagggacgataagaccctataaagtttatataccgaactaattaaagataattaagaagggtaaagtttattattagagaggtatttagttggggcgactaggatataaattaatttaactgttttaaattaaagtcaaaaatatttgagtttgtgatccttttttaggattagagaataagttactttagggataacagcgtaat

SEQ ID NO: 632 - DNA, 206 bp, Cherax preissii
  source 1..206: mol_type = genomic DNA
aagggacgataagaccctataaagtttatatgttgaactaattaaagataattaggaagagtaaagtttattattggttaggcatttagttggggcgactgaaatataagttaatttaactgttttaaattaaggtcaaagatgtttgagtttgtgatccctttttaggatgatagaataagttactttagggataacagcgtaat

SEQ ID NO: 633 - DNA, 209 bp, Munida spinosa
  source 1..209: mol_type = genomic DNA
aagggacgataagaccctatgaatctttatattatgtcactttaagttagttaatatcgtagatataatgataaggtgaggtgtattgtgttggggtgatgagaatataagaaataactgttctaaatttaagacaaaattgtttgtggtaataatagatcctttattgagattaaaagtttaagttactttagggataacagcgttat

SEQ ID NO: 634 - DNA, 209 bp, Munida asprosoma
  source 1..209: mol_type = genomic DNA
aagggacgataagaccctataaatctttatattagattatttttagttatttaatattgtgagtataacaataaagtggtatatattgtgttggggtgatgagaatataataaataactgttctaaaattaaaacaaaattatttgtataaataatagatcctttattgagattaaaagtttaagttactttagggataacagcgttat

SEQ ID NO: 635 - DNA, 210 bp, Munida leagora
  source 1..210: mol_type = genomic DNA
aagggacgataagaccctataaatctttatattaaattatttttagttgattaatattgtaaatataataataaaatatcaaatattgtgttggggcgacgagaatataataaattaactgttctaaaattaaaacaaaattgtttgtaaaaataatagatcctttataaagagtaaaagttcaagttactttagggataacagcgttat

SEQ ID NO: 636 - DNA, 210 bp, Munida alonsoi
  source 1..210: mol_type = genomic DNA
aagggacgataagaccctataaatctttatattaaattgtttaatatttaattaatattgtgtactagatatttaaataatatatattgtgttggggtgacgagaatataataaatagctgttctaaagtaaaaacaatttttattgtggtaatagaagatcctttattgagattgaaagtttaagttactttagggataacagcgttat

SEQ ID NO: 637 - DNA, 209 bp, Munida taenia
  source 1..209: mol_type = genomic DNA
aagggacgataagaccctataaatctttatattaaattattttcaattaattaatactgtaaatacaataataaaataataaatattatgttggggcgatgagaatataataaataactgttctaaaatttatacaaaattatttgttacaataatagatcctttatagagattaaaagtttaagttactttagggataacagcgttat

SEQ ID NO: 638 - DNA, 212 bp, Munida gordoae
  source 1..212: mol_type = genomic DNA
gagggacgataagaccctataaatctttatattaagttgttttttactaattaatattgtagatgaagtaataaaattatatatattgtgttggggcgatgagaatataaataaataactgttctagtgtagaaaaacaaaattatttggatattggtagatcctttagttaagattaaaagtttaagttactttagggataacagcgttat

SEQ ID NO: 639 - DNA, 209 bp, Munida zebra
  source 1..209: mol_type = genomic DNA
aagggacgataagaccctataaatctttatactaggatatttttagttaattaatattgtaaatataataataaaatgacatgtattgtgttggggtgatgagaatataataaataactgttctaagatgaaaacaaaattatttgtagtaataatagatcctttattgagattgtaagtttaagttactttagggataacagcgttat

SEQ ID NO: 640 - DNA, 209 bp, Munida distiza
  source 1..209: mol_type = genomic DNA
aagggacgataagaccctataaatctttatattaaactgttttcaattaattaatattgttaatataatagtaaaagagtaaatattatattggggcgataagaatatataaattaactgttctaaagttaaaacaaaattatttgtaaaaataatagatccttaataaagattaaaagttcaagttactttagggataacagcgttat

SEQ ID NO: 641 - DNA, 208 bp, Munida psamathe
  source 1..208: mol_type = genomic DNA
aagggacgataagaccctataaatctttatattaagttatttttagttaattaatattgtaaatgaaattacaaaataatatatattgtgttggggtgatgagaatataataaatatctgttctaaaataaaaacaatttttttgtgttagtagaagatcctttgttgagattaaaagtttaagttactttagggataacagcgttat

SEQ ID NO: 642 - DNA, 209 bp, Munida thoe
  source 1..209: mol_type = genomic DNA
gagggacgataagaccctataaatctttatattaggttattttatagttagttaatattgtaaatataataataaggtgaaatatattatgttggggtgatgagaatataataaataactgttctaagataaaaacaaaattgtttgtaaaatagtagatcctttgttgagattagaagtttaagttactttagggataacagcgttat

SEQ ID NO: 643 - DNA, 209 bp, Munida guttata
  source 1..209: mol_type = genomic DNA
aagggacgataagaccctataaatctttatattaaactgttttcaattaattaatattgttaatataatagtaaaatagtaaatattatattggggtgataagaatatataaattaactgttctaaaattaaaacaaaattatttgtagtaataatagatccttaataaagattaaaagttcaagttactttagggataacagcgttat

SEQ ID NO: 644 - DNA, 213 bp, Munida stia
  source 1..213: mol_type = genomic DNA
aagggacgataagaccctataaatctttatatcaaattatttttagttgattaatatagtagatattaataataaaataacacatattatattggggtgataagaatataaaaaataactgttctaaaattaaaacaaaattatttgtagtagttataatagatcctttattgagatccaaagtttaagttactttagggataacagcgttat

SEQ ID NO: 645 - DNA, 212 bp, Munida ommata
  source 1..212: mol_type = genomic DNA
aagggacgataagaccctataaatctttatattaagttaattttttagttaattaatattgtaaatataatagtaagatggcgtatattgtgttggggtgatgagaatataataaataactgttctaaaatataaacaatattgtttgtaaataatagtagatcctttattgagattaaaagtttaagttactttagggataacagcgttat

SEQ ID NO: 646 - DNA, 211 bp, Munida roshanei
  source 1..211: mol_type = genomic DNA
aagggacgataagaccctataaatctttatattaaactatttaaaattaattaatatgtaaatttaataaataaaatggaaaatattatattggggtgataagaatataaattatataactgttctaaaatttaaacaaaattatttgtacaaatagtagatccttttttaagattaaaaatttaagttactttagggataacagcgttat

SEQ ID NO: 647 - DNA, 210 bp, Munida compressa
  source 1..210: mol_type = genomic DNA
aagggacgataagaccctataaatctttatattagattatttttaagttatttaatattgtaaatataataataaaataatatatattgtgttggggtgatgagaatataatgaataactgttctaaaatgaaaacaaaattatttgtgaaaataatagatcctttattgagattaaaagtttaagttactttagggataacagcgttat

SEQ ID NO: 648 - DNA, 213 bp, Munida clinata
  source 1..213: mol_type = genomic DNA
aagggacgataagaccctataaatctttatattaaactaccttaaattaattaatattgtaaataataacggtaaggtagaaaatattatattggggtgataagaatataaaataatataactgttctaaaatttaaacaaaattatttgtaataataatagatccttttttaagatcaaaaatttaagttactttagggataacagcgttat

SEQ ID NO: 649 - DNA, 211 bp, Munida chydaea
  source 1..211: mol_type = genomic DNA
gagggacgataagaccctataaatctttatattaaattatttttagttaattaatattgtgaacataataataaagtaaggaaatattgtgttggggtgatgagaatataaataataactgttctaaaattaaaacaaaattatttgtaagtaataatagatcctttattgagattaaaagtttaagttactttagggataacagcgttat

SEQ ID NO: 650 - DNA, 209 bp, Munida compacta
  source 1..209: mol_type = genomic DNA
aagggacgataagaccctataaatctttatattagattatttttagttgtttaatattgtaaatataataataaaatgatatatattgtgttggggtgatgagaatataataaataactgttctaaaatgaaaacaatattatttgaatgaatagtagatcctttattgagattaaaagtttaagttactttagggataacagcgttat

SEQ ID NO: 651 - DNA, 209 bp, Munida eclepsis
  source 1..209: mol_type = genomic DNA
aagggacgataagaccctataaatctttatattagattatttttagttatttaatattgtaaatataataataaagtggtgtatattatgttggggtgatgagaatataataaataactgttctaaaataaaaacaaaattgtttgtatagataatagatcctttattgagattaaaagtttaagttactttagggataacagcgttat

SEQ ID NO: 652 - DNA, 210 bp, Munida tyche
  source 1..210: mol_type = genomic DNA
aagggacgataagaccctataaatcttaatattaagttatttttagttaattaatatctgtaaatacaataataaaatattaaatattatgttggggtgacaagaatataataaataactgttctaaaatttaaacaaaattatttgtaaaaaaaaaagatcctttttaaagattaaaagtttaagttactttagggataacagcgttat

SEQ ID NO: 653 - DNA, 213 bp, Munida philippinensis
  source 1..213: mol_type = genomic DNA
aagggacgataagaccctataaatctttatattaaattatttttattttattaataccgtagatataatgataaagtaataaatattatgttggggtgataagaatataaaaaattaactgttctaagattaaaacaaaattatttgaaaaaaaaataatagatcctttataaagattaaaagtttaagttactttagggataacagcgttat

SEQ ID NO: 654 - DNA, 210 bp, Munida armilla
  source 1..210: mol_type = genomic DNA
aagggacgataagaccctataaatctttatattaaattattttcaattaattaatattgtaaataaaataataaaataataaatattatattggggcgatgagaatataacaaaataactgttctacaattaatacaaaattatttgtaataataatagatcctttataaagattaaaagtttaagttactttagggataacagcgttat

SEQ ID NO: 655 - DNA, 209 bp, Munida mesembria
  source 1..209: mol_type = genomic DNA
aagggacgataagaccctataaatcttaatattaaattatttttagttaattaataattgtaaatataataataaaatattaaatattatgttggggtgacaagaatataataaataactgttctaaaattaaaacaaaattatttgtaaaaaaaatgatcctttataaagattaaaagttcaagttactttagggataacagcgttat

SEQ ID NO: 656 - DNA, 207 bp, Munida spilota
  source 1..207: mol_type = genomic DNA
aagggacgataagaccctataaatctttatattaaattatttttaattaattaatattgtaaatttaataataaaatattaaatattatgttggggcgacaagaatataataaataactgtctaaatttaaaacaaaattatttgtaataaaatagatcctttttaaagattaaaagtttaagttactttagggataacagcgttat

SEQ ID NO: 657 - DNA, 209 bp, Munida benguela
  source 1..209: mol_type = genomic DNA
aagggacgataagaccctataaatctttatattagattatttttagttatttaatattgtaaatatagtaataaaatgatatatattgtgttggggtgatgagaatataataaataactgttctaaaatgaaaacaaaattatttgaataaatagtagatcctttattgagattaaaagtttaagttactttagggataacagcgttat

SEQ ID NO: 658 - DNA, 208 bp, Munida endeavourae
  source 1..208: mol_type = genomic DNA
aagggacgataagaccctataaatctttatattaaattatttttaattagttaatattgtaaatataatagtaaggtaaagtatattgtgttggggtgatgagaatataagaaataactgttctaaaataaaaacaaaattatttgaagaaataatagatcctttgttgagattaaaagtttaagttactttagggataacagcgtta

SEQ ID NO: 659 - DNA, 207 bp, Munida agave
  source 1..207: mol_type = genomic DNA
aagggacgataagaccctataaatcttaatattaaattatttttagttaattaatagttgtaaatataataataaaatattaaatattatgttggggtgacaagaatataataaataactgttctaaaatttaaacaaaattatttgtaaaaaaagatcctttataaagattaaaagtttaagttactttagggataacagcgttat

SEQ ID NO: 660 - DNA, 210 bp, Munida idyia
  source 1..210: mol_type = genomic DNA
gagggacgataagaccctataaatctttatattaggttattttcagttgattaatattgtaaatataataataagataatatatattgtgttggggtgatgagaatataatgaaataactgttctaggatagaaacaaaattatttgaagtaataaatgatcctttattgagattaaaagtttaagttactttagggataacagcgttat

SEQ ID NO: 661 - DNA, 209 bp, Munida militaris
  source 1..209: mol_type = genomic DNA
aagggacgataagaccctataaatctttatattagattatttttagttatttaatattgtaaatataataataaagtgatttatattgtgttggggtgatgagaatataataaataactgttctaaaatgaaaacaaaattatttgtgtaaataatagatcctttattgagattaagagtttaagttactttagggataacagcgttat

SEQ ID NO: 662 - DNA, 213 bp, Munida flinti
  source 1..213: mol_type = genomic DNA
aagggacgataagaccctataaatctttatattaacttattttttatatattaatcttgtaaactccgtttaataaaataagatatattttgttggggcgacaagaatataaacacaactgttctaaatactaaacaaaactatttgttttaaattcatagatccttcatttaagattataagtttaagttactttagggataacagcgttat

SEQ ID NO: 663 - DNA, 209 bp, Munida congesta
  source 1..209: mol_type = genomic DNA
aagggacgataagaccctataaatctttatattagattatttttagttatttaatattgtaaatatagtaataaaataatatatattgtgttggggtgatgagaatataataaataactgttctaaaatataaacaaaattatttgaataaatagtagatcctttattgagattaaaagtttaagttactttagggataacagcgttat

SEQ ID NO: 664 - DNA, 210 bp, Munida rubridigitalis
  source 1..210: mol_type = genomic DNA
aagggacgataagaccctataaatctttatattagattatttttaagttgtttaatattgtaaatataataataaaataatatatattgtgttggggtgatgagaatataataaataactgttctaaaatgaagacaaaattatttgtataaataatagatcctttattgagattaaaagtttaagttactttagggataacagcgttat

SEQ ID NO: 665 - DNA, 209 bp, Munida iris
  source 1..209: mol_type = genomic DNA
aagggacgataagaccctataaatctttatattaaattatttttaatttgttaattatgtaaatttaataacaagataaaatatattatgttggggtgatgagaatatataatcaactgttctaaattaaaaacaaaattatttgaacaaaatagtagatccttattagagattaaaagtttaagttactttagggataacagcgttat

SEQ ID NO: 666 - DNA, 210 bp, Munida microphthalma
  source 1..210: mol_type = genomic DNA
aagggacgataagaccctataaatctttatattaaattattttaaattaattaatattgtaaataagataataaaataatatatattgtgttggggtgatgagaatataataaataactgttctaaaataaaaacaaagctatttgtataaaataatagatcctttactaagattaaaagtttaagttactttagggataacagcgttat

SEQ ID NO: 667 - DNA, 210 bp, Munida rufiantennulata
  source 1..210: mol_type = genomic DNA
gggggacgataagaccctataaatctttatattaagttatttttagttaattaatattgtaaatataataataaaatagcgtgtattgtgttggggtgatgagaatataataaataactgttctaaaatgtaaacaatattatttgtaaataatagtagatcctttattgagattaaaagtttaagttactttagggataacagcgttat

SEQ ID NO: 668 - DNA, 212 bp, Munida pusilla
  source 1..212: mol_type = genomic DNA
aagggacgataagaccctataaatctttatataaaattatctttaatttatttataatgtaagtttaatataaagataagatatattacattggggtgatgagaatatataataaactgttctagattcaaaacaaatttatttgtattataaaatagtagatccttttttaagattaaaagtttaagttactttagggataacagcgttat

SEQ ID NO: 669 - DNA, 209 bp, Munida remota
  source 1..209: mol_type = genomic DNA
aagggacgataagaccctataaatctttatattagattatttttagttatttaatattgtgaatataataataaagtggtatatattgtgttggggtgatgagaatataataaataactgttctaaaattaaaacaaaattatttgtataaataatagatcctttattgagattaaaagtttaagttactttagggataacagcgttat

SEQ ID NO: 670 - DNA, 208 bp, Munida leptosyne
  source 1..208: mol_type = genomic DNA
aagggacgataagaccctataaatctttatattaatttatttgtttattgattaataagtagatgatataataagataatatatattatgttggggtgatgagaatataataattaactgttctaagttgtaaacaaaattatttgaaattttatagatcctttattgagattaaaagtttaagttactttagggataacagcgttat

SEQ ID NO: 671 - DNA, 209 bp, Munida rosula
  source 1..209: mol_type = genomic DNA
aagggacgataagaccctataaatctttatattagattatttttagttatttaatattgtaaatataataataaagtgatatatattgtgttggggtgatgagaatataataaataactgttctaaaatgaaaacaaaattatttgtgtaaataatagatcctttattgagattaaaagtttaagttactttagggataacagcgttat

SEQ ID NO: 672 - DNA, 166 bp, Munida munin
  source 1..166: mol_type = genomic DNA
tctttatattaaactatttaaaattaattaatatgtaaatttaataaataaaatggaaaatattatattggggtgataagaatataaattatataactgttctaaaatttaaacaaaattatttgtacaaatagtagatccttttttaagattaaaaatttaagtt

SEQ ID NO: 673 - DNA, 214 bp, Munida valida
  source 1..214: mol_type = genomic DNA
aagggacgataagaccctataaatctttatattaagttattttaaattaattaatgttgtaaataaaataataaagtaatatatattgtgttggggtgatgagaatataataaataactgttactaaatttaaaaacaagttatttgaatttaaaataatagattcctttttgagtattaaaagtttaagttactttagggataacagtcgtta

SEQ ID NO: 674 - DNA, 208 bp, Munida proto
  misc_feature 14: note = n is a, c, g, or t
  source 1..208: mol_type = genomic DNA
gagggacgataagnccctataaatcttaatattaaattatttttaattaattaatattgtaaatataataataaaatattaaatattatgttggggtgataagaatataaaaaataactgttctaaatttaaaacaaaattatttgaaaataatagatccttttttaaagattaaaagttcaagttactttagggataacagcgttat

SEQ ID NO: 675 - DNA, 209 bp, Enriquea leviantennata
  source 1..209: mol_type = genomic DNA
aagggacgataagaccctataaagctttatattattttttttttatagattattgttgtggatataataataaaataaggaatattatattggggtgataagggtataaaattaactgctctaaaattaaatcaaatttatttgtaaaataataatagatcctttattaagattaaaagtttaagttactttagggataacagcgttat

SEQ ID NO: 676 - DNA, 163 bp, Munida multilineata
  source 1..163: mol_type = genomic DNA
tcttaatattaaactatttttagttaattaattaattgtaaatataaataataaaatattaaatattatgttggggtgacaagaatataaaaaataactgttctaaatttaaaacaaaattatttgaaaaaaagatcctttataaagattaaaagttcaagtt

SEQ ID NO: 677 - DNA, 209 bp, Munida pagesi
  source 1..209: mol_type = genomic DNA
aagggacgataagaccctataaatctttatattatattatttttagttaattaatattgtaaatataataataaggtaatatatattatgttggggtgataagaatataataaataattgttctaaaatgaaaacaaaattgtttgtgttaataatagatcctttattgagattaaaagtttaagttactttagggataacagcgttat

SEQ ID NO: 678 - DNA, 212 bp, Munida stomifera
  source 1..212: mol_type = genomic DNA
gagggacgataagaccctataaatctttatattaagttgtttttagctgattaatgttgtagattcagtattaagacaaaatatattatgttggggtgatgagaatacaatagatacctgttctaaaatatcaaacaaaaatttttgtgtgagtagaagatcctctttattgagatcaggagtttaagttactttagggataacagcgttat

SEQ ID NO: 679 - DNA, 208 bp, Munida quadrispina
  source 1..208: mol_type = genomic DNA
gggacgataagaccctataaatctttatattaaattgtttttagtttattaataatgtaatttagataataaggcaaaatatattgtgttggggtgatgggaatataaaaaattaactgttctgaaataaaaacaaatttgtttggcttaataatagatccttttttaagattaaaagtttaagttactttagggataacagcgttat

SEQ ID NO: 680 - DNA, 210 bp, Munida tiresias
  source 1..210: mol_type = genomic DNA
aagggacgataagaccctataaatctttatattaagttgtttttagttaatttatattgtaaataagataataaagtaaaatatattgtgttggggtgatgagaatataataaataactgttctaaaataaaaacaaagctatttgtattaaataatagatcctttattaagattaaaagtttaagttactttagggataacagcgttat

SEQ ID NO: 681 - DNA, 209 bp, Munida psylla
  source 1..209: mol_type = genomic DNA
atgggacgataagaccctataaatctttatattaagttatttttagttaattaatattgtaaatataataataaaataatgtatattgtgttggggtgatgagaatataataaataactgttctaaaatgtaaacaatattgtttgtaataatagtagatcctttattgagattaaaagtttaagttactttagggataacagcgttat

SEQ ID NO: 682 - DNA, 209 bp, Munida heteracantha
  source 1..209: mol_type = genomic DNA
aagggacgataagaccctataaatctttatattaaactgttttcaattaattaatattgtaaatataataataaaatagtaaatattatgttggggtgatgagaatataagaaataactgttctaaaattaaaacaaaattgtttgtaataataatagatcctttataaagattaaaagtttaagttactttagggataacagcgttat

SEQ ID NO: 683 - DNA, 214 bp, Paralomis formosa
  source 1..214: mol_type = genomic DNA
aaaagacgataagaccctataaatctttacaagaaatatattttatgttttagcttataagtatataagaaataaaaatataagtttgttgcgctggggcggcgtagatatataaataaactgtctatggtttaaatacaataattattgcttaatacaaattgatccttaaatagattaaaagattaagatactttagggataacagcgttat

SEQ ID NO: 684 - DNA, 214 bp, Paralomis spinosissima
  source 1..214: mol_type = genomic DNA
aaaagacgataagaccctataaatctttacaagaaatatattttatgttttagcttataagtgtataagaaataaaaatataagtttgttgcgctggggcggcgtagatatataaataaactgtctatgatttaaatacaataattattgyttaatacarattgatccttaaatagattaaaagattaagatactttagggataacagcgttat

SEQ ID NO: 685 - DNA, 214 bp, Paralomis birsteini
  source 1..214: mol_type = genomic DNA
aaaagacgataagaccctataaatctttacaagaaatatattttatgttttagcttataagtgtataagaaataaaaatataagtttgttgcgctggggcggcgtagatatataaataaactgtctatggtttaaatacaataattattgcttaatacagattgatccttaaatagattaaaagattaagatactttagggataacagcgttat

SEQ ID NO: 686 - DNA, 214 bp, Paralomis hirtella
  source 1..214: mol_type = genomic DNA
aaaagacgataagaccctataaatctttacaagaaatatattttatgttttagcttataagtgtataagagataaaaatataagtttgttgcgctggggcggcgtagatatataaataaactgtctgtggtttaaatacaataattattgcttaatacaaattgatccttaaatagattaaaagattaagatactttagggataacagcgttat

SEQ ID NO: 687 - DNA, 207 bp, Scyllarus subarctus
  source 1..207: mol_type = genomic DNA
aagggacgataagaccctataaatctttatacttgattttatagagcaaaatattaaaataatttttgttcaataggtttgggtattttgttggggcgacaggagtataaaatgtaactgctctaaaatgatagttagttgatatttagttaagtgatcctttaataaagatgttagtttaagttactttagggataacagcgtaat

SEQ ID NO: 688 - DNA, 207 bp, Scyllarus pygmaeus
  source 1..207: mol_type = genomic DNA
gggggacgataagaccctataaatctttatatttaagcttataaaataaacgattaaaataatttttgtttaataagcttatatattttgttggggcgccagaagtataaaatgtaactgctttaaaataatagttaaatgatatttagttaagtaatcctttaataaagatgttagtttaagttactttagggataacagcgtaat

SEQ ID NO: 689 - DNA, 206 bp, Scyllarus chacei
  source 1..206: mol_type = genomic DNA
gggggacgataagaccctatgaatctttatatttcggtttataagataaaaaattaatataatttttgtcttattaatctttttattttgttggggcgacagaagcgtaaactgtaaccgctttgaaaaaatagttaatgatatttagttaagtgacccttctatgggggtatcagtataagttactctagggataacagcgtaat

SEQ ID NO: 690 - DNA, 208 bp, Scyllarus caparti
  source 1..208: mol_type = genomic DNA
aggggacgataagaccctataaatctttatatttgggtttgtgaaataaaaaattaaaataatttttgtttaataagcttgggtattttgttggggcgacaagagtataaaaatgtaactgctttgaaataatagttaattggtgtttagttaattgatcctttagtagagatgtcagtttaagttactttagggataacagcgtaat

SEQ ID NO: 691 - DNA, 205 bp, Scyllarus americanus
  source 1..205: mol_type = genomic DNA
gggggacgataagaccctatgaatctttatttttggcttataaaataaaaaaataatatcaatttttgttttatcggtttcaatattttgttggggcgacggaagcgtaaattgtaaccgctttatattaatagttaacgatatttagttaagtgatcctttatagagatattagtataagttactctagggataacagcgtaat

SEQ ID NO: 692 - DNA, 218 bp, Episesarma palawanense
  source 1..218: mol_type = genomic DNA
aaaagacgataagaccctataaaacttaataaaaattgatatttaaataaaatttattaataaatatttagtaattaatttttattttattggggtgataatggtaaaatggatattaactgttaataaagtttataacaaaaattattgagtaagtacaataaatgatccttattaaagattaaaagtttaagttactttagggataacagcgttat

SEQ ID NO: 693 - DNA, 222 bp, Episesarma singaporense
  source 1..222: mol_type = genomic DNA
aaaagacgataagaccctataaaacttaatataaattgattatttgagtaaaattttattgtataaatatttaataattaatttatattttattggggtgataatggtaaaatgattattaactgttaattattttatgacaaaaattattgaataaatgtgtaataaatgatccttattaaagattaaaagtttaagttactttagggataacagcgttat

SEQ ID NO: 694 - DNA, 199 bp, Austropotamobius fulcisianus orientalis
  source 1..199: mol_type = genomic DNA
gggggacgataagaccctataaaactttatattttaaaaatattaattaattttgtatgagaaggctatttttaaatattttattggggtgatagggatataataaaagataactgtttcttttttttacagaggtgtttgagtaaaagatcctaattagggaaagaaggttaagttactttagggataacagcgtaat

SEQ ID NO: 695 - DNA, 212 bp, Achelous tumidulus
  source 1..212: mol_type = genomic DNA
gggggacgataagaccctataaagcttcataaatttatagattttatgcatttaatataaaaatagtttctgcagtatttatttggttggggcgacaaaagtataatttttaataactgctatttgtaagttacaattatatttggttagtgagtaattgatccttctgtgaggagtaaaagttcaagttactttagggataacagcgtcat

SEQ ID NO: 696 - DNA, 212 bp, Achelous asper
  source 1..212: mol_type = genomic DNA
gggggacgataagaccctataaagctttataaattcaagaattttatatatttagtataaaaataattttctcgatgtttatttggttggggcgacaaaagtataattttgaataactgctagttcagacatacaaaaatctctgatttataaataatggatccctttatgaggattaaaagttcaagttactttagggataacagcgttat

SEQ ID NO: 697 - DNA, 213 bp, Achelous sebae
  source 1..213: mol_type = genomic DNA
gagggacgataagaccctataaagctttataagtttataaattttatacatttgtaaataaaaataatttttttaatacttatttggttggggcgacaaaagtgtaatttaaaataaccgctaatatattaatacaataatttttggctaactaagtaatggatcccttttaaggaataaaagaccaagttactttagggataacagcgttat

SEQ ID NO: 698 - DNA, 188 bp, Portunus acuminatus
  source 1..188: mol_type = genomic DNA
ggaagacgataagaccctataaagctttataagtttataaattttgtatatttaacctaaaaataattttttaggtgcttatttggttggggcgacaaaagtataattacattgattttttatttaaaaataatggatccttctgtgaagaataaaagttcaagttactttagggataacagcgtaat

SEQ ID NO: 699 - DNA, 210 bp, Achelous tuberculatus
  source 1..210: mol_type = genomic DNA
gggggacgataagaccctataaagctttataagtttatggattttatagatttaraataaagataatttcctgaatatttatttggttggggcgacaaaagtataatttaaataactgctagttgaagattacagtgatttttgattctaaaataatggatcccttataaggattaaaaaatcaagttactttagggataacagcgttat

SEQ ID NO: 700 - DNA, 212 bp, Achelous iridescens
  source 1..212: mol_type = genomic DNA
gggggacgataagaccctataaagctttataaatttatgaattttatttatttaatataaaaataatttttacaatatttattaggttggggcgacaaaagtataattcgaaataactgctattttaaaattacaatagtttttgttttaaaagtaattgatccctttatgaggagtaaaagttcaagttactttagggataacagcgttat

SEQ ID NO: 701 - DNA, 211 bp, Portunus xantusii
  source 1..211: mol_type = genomic DNA
gggggacgataagaccctataaagcttcataaatttataaattttatgtatytaatataaagataatttttgtaacgtttatttggttggggcgacaaaagtataatttcaaataactgctaggtttgatttacaatgatagttgattaataagtaattgatcccttgtgaggaataaaagttcaagttactttagggataacagcgttat

SEQ ID NO: 702 - DNA, 211 bp, Achelous depressifrons
  source 1..211: mol_type = genomic DNA
gggggacgataagaccctataaagctttataaatttattaattttgtatatttaaaataaaaacaatcttttcaatatttatttggttggggcgacaaaagtgtaattaaagtaaccgctaataatataattacaataatttttgattagcaattaatggatcccttttaaggaataaaagatcaagttactttagggataacagcgttat

SEQ ID NO: 703 - DNA, 212 bp, Achelous rufiremus
  source 1..212: mol_type = genomic DNA
aggggacgataagaccctataaagctttataagtttatggattttatatatttaatataaaaataattttttaaatatttatttggttggggcgacaaaagtataattggaaataactgctaactttaagtaccactacatcttgtataaaaaataatcggattctttataaagagtaaaagttcaagttactttagggataaaagcatcat

SEQ ID NO: 704 - DNA, 212 bp, Achelous gibbesii
  source 1..212: mol_type = genomic DNA
gggggacgataagaccctataaagctttataaatttaaaaattttatatatttatcataaagataatttttcaatgtttatttggttggggcgacaaaagtataatttaaaataactgctagtttgaaaacacaataatttttgttttaaaaagtaatggatccctttatgaggattaaaagttcaagttactttagggataacagcgtcat

SEQ ID NO: 705 - DNA, 211 bp, Portunus minimus
  source 1..211: mol_type = genomic DNA
gggggacgataagaccctataaagctttataaatttataaattttatgtatttaatataaagataatttttgtaacgtttatttggttggggcgacaaaagtataatttcaaataactgctaggtttggtttacaatgatagttgattaataagtaattgatcccttgtgaggaataaaagttcaagttactttagggataacagcgttat

SEQ ID NO: 706 - DNA, 212 bp, Achelous stanfordi
  source 1..212: mol_type = genomic DNA
ggaagacgataagaccctataaagctttataagtctacgaattctgtagatctgatataaaaataatttttttgatacttatttggttggggcgacaaaagtataatttaaataactgctaatttaaaattacaatgatttttgattataaaaataatggatctttctctgaaaaatgaaagttcaagttactttagggataacagcgtaat

SEQ ID NO: 707 - DNA, 211 bp, Achelous brevimanus
  source 1..211: mol_type = genomic DNA
gggggacgataagaccctataaagctttataaatttataaattttatgtatttaatataaagataatttttgtaacgtttatttggttggggcgacaaaagtataattttaaataactgctaggtttgatttacagcgatagttgattaataagtaattgatcccttgtaaggaataaaagttcaagttactttagggataacagcgttat

SEQ ID NO: 708 - DNA, 211 bp, Portunus affinis
  source 1..211: mol_type = genomic DNA
gggggacgataagaccctataaagcttcataaatttataaattttatgtatctaatataaagataatttttgtaacgtttatttggttggggcgacaaaagtataatttcaaataactgctaggtttgatttacaatgatagttgattaataagtaactgatcccttgtgaggaataaaagttcaagttactttagggataacagcgttat

SEQ ID NO: 709 - DNA, 213 bp, Achelous angustus
  source 1..213: mol_type = genomic DNA
gggggacgataagaccctataaagctttataaatttatagattttatttatttaataataaaaataatttctacagtgtttatttggttggggcgacaaaagtataattttgaataactgctagttctaagttacaacaatatttgatttgtaagtaattgatccttctatgaggagtaaaagttcaagttactttagggataacagcgttat

SEQ ID NO: 710 - DNA, 213 bp, Achelous binoculus
  source 1..213: mol_type = genomic DNA
gggggacgataagaccctataaagctttataaattttatagactttatatatttaaaataaaaataatttctaaaacatttatttggttggggcgacaaaagtataatttcaaataactgctattttaaaattacaataatttttgttttatgaataattgatccttctatgaggagtaaaagttcaagttactttagggataacagcgtcat

SEQ ID NO: 711 - DNA, 216 bp, Oratosquillina inornata
  source 1..216: mol_type = genomic DNA
gagggacgataagaccctataaagctttatattacatttcttttttataaattatttcgttattaacttattttagaatttgtattattttactggggcggtaaaagtatattaaagattaactgcttttgttttgtaaaacaattatagttgaaaataaattgatccattattaatgatcataagaacaagttactttagggataacagcgtaat

SEQ ID NO: 712 - DNA, 216 bp, Oratosquillina asiatica
  source 1..216: mol_type = genomic DNA
gagggacgataagaccctataaagctttatgttattcttcttttttataaattatttaattattaacttatttcagaattaatatcattttactggggcggtaaaagtataataaagattaactgcttttgttttgtctaacaataatagttgaaaatagattgatccattattaatgatcataagaacaagttactttagggataacagcgtaat

SEQ ID NO: 713 - DNA, 216 bp, Oratosquillina anomala
  source 1..216: mol_type = genomic DNA
gagggacgataagaccctataaagctttatattacatttcttttttataaattatttcgttattaacttattttagaatttgtattattttactggggcggtaaaagtataatgaagattaactgcttttgttttgtaaaacaattatagttgaaaataatttgatccattattaatgatcataagaacaagttactttagggataacagcgtaat

SEQ ID NO: 714 - DNA, 217 bp, Oratosquillina perpensa
  source 1..217: mol_type = genomic DNA
ggagggacgataagaccctataaagctttatgttacatttcttttttataaattattttgttattaacttattttagaatttgtatcattttactggggcggtaaaagtataataaagattaactgcttttgttttgtataacaattttagttggaaataaattgatccattattaatgatcataagaacaagttactttagggataacagcgtaat

SEQ ID NO: 715 - DNA, 215 bp, Erugosquilla graham
  source 1..215: mol_type = genomic DNA
gagggacgataagaccctataaagctttatgatattcttcttttttataaattgtcttgttattaacttattttagaattaatatcattttactggggcggtaaaagtataaaaagattaactgcttttgttttgtataacaatcttagttggaaataaattgatccattattaatgatcataagaacaagttactttagggataacagcgtaat

SEQ ID NO: 716 - DNA, 214 bp, Busquilla quadraticauda
  source 1..214: mol_type = genomic DNA
gagggacgataagaccctataaagctttatgatatttctctttcttataaatttttgttattaacctattttagagttaatatcattttactggggcggtaaaagtataataaagattaactgcttttgttttgtacaacaattttagttggaaataaattgatccattattaatgatcataagaccaagttactttagggataacagcgtaat

SEQ ID NO: 717 - DNA, 213 bp, Kempella stridulans
  source 1..213: mol_type = genomic DNA
gagggacgataagaccctataaagctttataatacctttcttttttataaaattttttgttgttaacatattttagaatttgtattattttactggggcggtaaaagtataataaataactgcttttattttgtgtaacaattttagttgatgataaattgatccattattaatgattataagaacaagttactttagggataacagcgtaat

SEQ ID NO: 718 - DNA, 216 bp, Gonodactylaceus graphurus; identical to SEQ ID NO: 368
  source 1..216: mol_type = genomic DNA

SEQ ID NO: 719 - DNA, 215 bp, Gonodactylaceus randalli
  source 1..215: mol_type = genomic DNA
aagtgacgataagaccctataaagctttatagtaatttctttttatttaaaatataaaattttaacctttttaaagattattactattttactggggcggtaaaagtatattttgattaactgcttttgttttaattaacaacgatagttgaatagtcatgtgatccattattaatgatcaaaagtacaagttactttagggataacagcgtaat

SEQ ID NO: 720 - DNA, 212 bp, Carcinus aestuarii
  source 1..212: mol_type = genomic DNA
gggggacgataagaccctataaaactttatatacacaacaatacagttaaattaaactataaaaacttattagataaaatatatttagttggggcgactgaggtataatttatagtaactgattaagagctagacaaataatatttgattaattttaattgatcctttttaaagattaaaagattaagttactttagggataacagcgtaat

SEQ ID NO: 721 - DNA, 214 bp, Menippe rumphii
  source 1..214: mol_type = genomic DNA
gagggacgataagaccctataaagctttataaattatttaaatttgattgaattaatataataaaattttgatttggattaatttattttgctggggcggcataggtataaattatattaactgcttagtagaaatacaataatagttgagttgacattaaaagatcctttttaaagatttaagaacaagttactttagggataacagcgttat

SEQ ID NO: 722 - DNA, 214 bp, Menippe nodifrons
  source 1..214: mol_type = genomic DNA
gagggacgataagaccctataaagctttatatattatctgaatttgagtaaattgatttataaaattttaatttggagtaatttattttgctggggcggcataggtataagttatattaactgcttaataaaaatacaattatagttgagttattaattaaaagatcctttttaaagatttgagaataagttactttagggataacagcgttat

SEQ ID NO: 723 - DNA, 213 bp, Menippe adina; identical to SEQ ID NO: 492
  source 1..213: mol_type = genomic DNA

SEQ ID NO: 724 - DNA, 198 bp, Procambarus liberorum
  source 1..198: mol_type = genomic DNA
aagggacgataagaccctataaaactttatatttgtaaattaatagctaattttatttaaatatattatttaaaaatattttgttggggtgacaaggataaaattaaaagtaactgtccttttttattacaataatgtttggtttaatgatccttaaatggatttaagattaagttactttagggataacagcgtaat

SEQ ID NO: 725 - DNA, 200 bp, Procambarus toltecae
  source 1..200: mol_type = genomic DNA
aagggacgataagaccctataaaactttatatttataaggtagaaaattagttttatttaatgatgctactttggagtatttggttggggtgacaaggataaaatataaaataactgtctttttttttctacagttatatttgatttaatgatcctaaaagggattaaagattaagttactttagggataacagcgttat

SEQ ID NO: 726 - DNA, 200 bp, Procambarus curdi
  source 1..200: mol_type = genomic DNA
aagggacgataagaccctataaaactttatatttataaattaatagttaattttatttaaatatattattttaaaatattttgttggggtgacaaggataaaattaaagataactgtccttttttattacagtaatatctggttaggtgatccttaaaatggattataagattaagttactttagggataacagcgtaat

SEQ ID NO: 727 - DNA, 201 bp, Procambarus digueti
  source 1..201: mol_type = genomic DNA
aagggacgataagaccctataaaactttatatctagaggtagtaggataattttatttaaggtgtattattttaagatatttggttggggtgaccaggataaaatgaaagataactgtcttatatattacaataatttttgggttgttgatcctacaaagggattaaaagattaagttactttaggcgataacagcgtaat

SEQ ID NO: 728 - DNA, 198 bp, Procambarus nigrocinctus
  source 1..198: mol_type = genomic DNA
aagggacgataagaccctataaaactttatatttataagtagtagttaattttatttgagtatattattttaaagtattatgttggggtgacaaggataaaattaaaaataactgtctttttttattacagtaatgtttggtttaatgatccttaaatggattataagattaagttactttagggataacagcgtaat

SEQ ID NO: 729 - DNA, 198 bp, Procambarus versutus
  source 1..198: mol_type = genomic DNA
aagggacgataagaccctataaaactttatattcataaattagtagttatttttattttaaagtattgttttagaatatttggttggggtggcaaggataaaatatttaataactgtcttttttttttacaataatgtttggtttaatgatcctaaaagggatttaagattaagttactttagggataacagcgtaat

SEQ ID NO: 730 - DNA, 198 bp, Procambarus gibbus
  source 1..198: mol_type = genomic DNA
aagggacgataagaccctataaaactttatatttgcaaggtagtagaaaattttattttaatttattattttaaagtatttggttggggtgacaaggataaaatataaaataactgtcttttttttttacagtaatgtttggtttaatgatcctaaaaaggattaaagattaagttactttagggataacagcgtaat

SEQ ID NO: 731 - DNA, 201 bp, Cambarus pecki
  source 1..201: mol_type = genomic DNA
aagggacgataagaccctataaaactttatatttaagaatgattaatgagttttgttttaaggttattattttgagatatttggttggggcgacaagaatagaagaaaaagtaactgttttttttttaataacaaaaatgtttgggttaatgatcctaaaatggattaaagattaagttactttagggataacagcgtaat

SEQ ID NO: 732 - DNA, 198 bp, Procambarus geminus
  source 1..198: mol_type = genomic DNA
aagggacgataagaccctataaaactttatatttataagatagtagctagttttatttaaaagtattattttggagtatttggttggggtgacaaggataaaatataaaataactgtcttttttttttacagtaatgtttggtttaatgatcctaaaaaggattaaagattaagttactttagggataacagcgtaat

SEQ ID NO: 733 - DNA, 168 bp, Charybdis acuta
  source 1..168: mol_type = genomic DNA
actttataaatttaatgattttatatattttatataaagataatttttggaaaatttatttggttggggcgacaatggtataatttaaataactgctattaagtttaacaaaaatatttgcttgaaatactgataattgatcctttataaggattttaagatcaagtt

SEQ ID NO: 734 - DNA, 198 bp, Creaserinus fodiens
  source 1..198: mol_type = genomic DNA
aagggacgataagaccctataaaactttatatttaaaggtataaattatttttatttatgtgttggtatctaaaagtatttggttggggcgacaagggcagaatattaagtaactgtctttttttttgacaataatatttggtttaatgatcctaaaaaggattaaagattaagttactttagggataacagcgtaat

SEQ ID NO: 735 - DNA, 199 bp, Fallicambarus jeanae
  source 1..199: mol_type = genomic DNA
aagggacgataagaccctataaaactttatatttataaatggtagacaattttgttttggggtattattttaaaatatttggttggggtgacaaggataaaataaaaaataactgtcttttttttttacagtgatatttggtttaatgatcctagaagtggattataagattaagttactttagggataacagcgtaat

SEQ ID NO: 736 - DNA, 198 bp, Creaserinus gordoni
  source 1..198: mol_type = genomic DNA
aagggacgataagaccctataaaactttatatttaaaggtataggttatttttatttgtgggttattatctaaaaatattgggtgggggcgacgaggatagaatattaagtaactgtctttttttaaaacagtaatatttgatttaatgatcctaaaagggattaaagattaagttactttagggataacagcgtaat

SEQ ID NO: 737 - DNA, 197 bp, Creaserinus caesius
  source 1..197: mol_type = genomic DNA
gagggacgataagaccctataaaactttatatttgagggtggaaattatttttatttggaggttgatatctaaaatattttgttggggcgacaaggataaaatattaagtaactgtcttttttttttacaatgatatttggtttaatgaccctaaaaaggattaaagattaagttactttagggataacagcgtaat

SEQ ID NO: 738 - DNA, 199 bp, Fallicambarus dissitus
  source 1..199: mol_type = genomic DNA
aagggacgataagaccctataaaactttatatttatgaatgatagttaattttgttttggaatattattttaaaatatttagttggggtgacaaggataaaatatgaaataactgtctttttttttttacagtgatatttggtttaatgatcctaaagtggattataagattaagttactttagggataacagcgtaat

SEQ ID NO: 739 - DNA, 199 bp, Creaserinus danielae
  source 1..199: mol_type = genomic DNA
aagggacgataagaccctataaaactttatatttaaggatatagactatttttatttgtgaactattatcaaaagtatttggttggggtgacagggatagaatataaagtaactgtcctttttttttaacaataatatttgacttagcgatccctgggaaggattaaagattaagttactttagggataacagcgtgat

SEQ ID NO: 740 - DNA, 198 bp, Fallicambarus oryktes
  source 1..198: mol_type = genomic DNA
aagggacgataagaccctataaaactttatatttaaggatatagactatttttatttgtaaactattatcaaaagtatttggttggggcgacagggatagaatataaagtaactgtccttttttttaacaataatatttgacttaacgatccctgggaaggattaaagattaagttactttagggataacagcgtgat

SEQ ID NO: 741 - DNA, 200 bp, Fallicambarus byersi
  source 1..200: mol_type = genomic DNA
aagggacgataagaccctataaaactttatattttaaggtatagattatttttgtttataggttgttatctgaaaatatttggttggggcgacagggataaaatgagaggtaactgttttttttttattacaacaatgtttgatttaatgatcctgaaaaggattaaaagattaagttactttagggataacagcgtaat

SEQ ID NO: 742 - DNA, 198 bp, Creaserinus burrisi
  source 1..198: mol_type = genomic DNA
aggggacgataagaccctataaaactttatatttaagggtataagttgtttttgtttgtaagttattatctgagagtatttggttggggcgacaggaataggatataaagtaactgttcttttttttgacaataatatttggtttaatgatcctaaaaaggattgaagattaagttactttagggataacagcgtaat

SEQ ID NO: 743 - DNA, 197 bp, Creaserinus gilpini
  source 1..197: mol_type = genomic DNA
gagggacgataagaccctataaaactttatatttgaaggtagaagttatttttatttgaaagttaatatctaaaatattttgttggggcgacaaggataaaatattaagcaactgtcttttttttttacaatgatatttggtttaatgatcctaaaaaggattaaagattaagttactttagggataacagcgtaat

SEQ ID NO: 744 - DNA, 199 bp, Fallicambarus harpi
  source 1..199: mol_type = genomic DNA
aagggacgataagaccctataaaactttatatttataaatagtagctaattttgttttgaaatattattttaaaatattttgttggggtgacaaggatataacagaaaataactgtcttttatgtttacagtaatgtctggtttaatgatcttaagaatagattataagattaagttactttagggataacagcgtaat

SEQ ID NO: 745 - DNA, 199 bp, Fallicambarus macneesei
  source 1..199: mol_type = genomic DNA
aagggacgataagaccctataaaactttatattttatgaatagtagttaattttattttataatattatttaaaagtatttggttggggtgacaaggataaaataaaaaataactgtcttttttttttacagtaatgtttggtttaatgatcctaaaatggattgaaagattaagttactttagggataacagcgtaat

SEQ ID NO: 746 - DNA, 198 bp, Fallicambarus petilicarpus
  source 1..198: mol_type = genomic DNA
gagggacgataagaccctataaaactttatatttataaatggtagttaattttattttagaatattattttggaatatttggttggggtgacaaggataaaatatgaaataactgtcttttttttttacagtgatatttggtttagtgatcctgaaatggattataagattaagttactttagggataacagcgtaat

SEQ ID NO: 747 - DNA, 197 bp, Fallicambarus wallsi
  source 1..197: mol_type = genomic DNA
gagggacgataagaccctataaaactttatatttataagtggtagttaattttattttataatattattttgaaatatttggttggggtgacgaggataaaataaaaaataactgtctttttttttaacagtaatgtttggtatgatgatcctaaaacggattaaagattaagttactttagggataacagcgtaat

SEQ ID NO: 748 - DNA, 198 bp, Fallicambarus strawni
  source 1..198: mol_type = genomic DNA
aagggacgataagaccctataaaactttatatttataaatggtagttaattttgttttaaaatgttattaaaaaatattttgttggggtgacaaggataaaatagaaaataactgtccttttttattacagtaatgtttggtttaatgatcctaaaaaggattataagattaagttactttagggataacagcgtaat

SEQ ID NO: 749 - DNA, 198 bp, Fallicambarus devastator
  source 1..198: mol_type = genomic DNA
aagggacgataagaccctataaaactttatatttataagtggtaattaattttatttaggaatattattttaaaatatttggttggggtgacaaggataaaagtaaaaataactgtccttttttttttcagtaatatctggtttgatgatcctaaaatggattataagattaagttactttagggataacagcgtaat

SEQ ID NO: 750 - DNA, 199 bp, Fallicambarus houstonensis
  source 1..199: mol_type = genomic DNA
aagggacgataagaccctataaaactttatattttatgaatggtagttaattttattttgtaatattatttaaaagtatttggttggggtgacaaggataaaataaaaaataactgtcttttttttttacagtaatgtttggtttaatgatcctaagatggattgaaagattaagttactttagggataacagcgtaat

SEQ ID NO: 751 - DNA, 197 bp, Fallicambarus hortoni
  source 1..197: mol_type = genomic DNA
aggggacgataagaccctataaaactttatatttaaggtggaaattatttttatttgttaagtactatttaaaaatatttggttggggcgacaaggatagaatgttaagtaactgtctttttttttaacaataatatttggtttaatgatcctaaaaaggattaaagattaagttactttagggataacagcgtaat

SEQ ID NO: 752 - DNA, 212 bp, Arenaeus cribrarius
  source 1..212: mol_type = genomic DNA
gagggacgataagaccctataaagctttataagttaaagtattttgtatattttatattataataatgatttattgagtacttatttggttggggcaacaagagtataataaaaagtaactgctaatgtttaaaacaattatatttgatttaagagtaattgatcccccaggggattaaaagatcaagctactttagggataacagcgtaat

SEQ ID NO: 753 - DNA, 200 bp, Cambarus tenebrosus
  source 1..200: mol_type = genomic DNA
aagggacgataagaccctataaaactttatattgaaaagtgataggtatattttgtttaaagaattattactttaaagtatttggttggggtgacaagaatagaagagaaagtaactgttttttttttatacaataatgtttgggttagtgatcctaaaatggattaaagattaagttactttagggataacagcgtaat

SEQ ID NO: 754 - DNA, 199 bp, Cambarus deweesae
  source 1..199: mol_type = genomic DNA
aagggacgataagaccctataaaactttatatttaggggtggaaattagttttatttaaaggttactattttataatatttggttggggcgacagggatagaaaaaaagttaactgtctttttttttttacagtaatatttggttgaatgatcctaaaatggattaaagattaagttactttagggataacagcgtaat

SEQ ID NO: 755 - DNA, 200 bp, Cambarus striatus
  source 1..200: mol_type = genomic DNA
aagggacgataagaccctataaaactttatattaaaaagtgatgggtaagttttgtttaaaggttattactttaaagtatttggttggggtgacaggaatagaatagaaagtaactgttcttttttttttacagtagtgtttgagttagtgatcctaaaatggattaaagattaagttactttagggataacagcgtaat

SEQ ID NO: 756 - DNA, 200 bp, Cambarus graysoni
  source 1..200: mol_type = genomic DNA
aagggacgataagaccctataaaactttatattgaaaagtggtaggtatgatttgtttaaaaggttactactttaaagtatttggttggggtgacaaaaatagaatagaaagtaactgtttttttttttaacaataatgtttgggttagtgatcctaaaatggattaaagattaagttactttagggataacagcgtaat

SEQ ID NO: 757 - DNA, 198 bp, Cambarus monongalensis
  source 1..198: mol_type = genomic DNA
aagggacgacaagaccctataaaactttatattgcaaagtgattagtaagttttatttaaaggttattattttgaaatatttggttggggtgacaaggattgaagaaaaagtaactgtatttttttttacaataatgtttggtttagtgatactaaaatggattaaagattaagttactttagggataacagcgtagt

SEQ ID NO: 758 - DNA, 199 bp, Cambarus pyronotus
  source 1..199: mol_type = genomic DNA
aagggacgataagaccctataaaactttatatttaaaagtgataggtaggttttgttttaaggttatttctttaaaatatttggttggggtgacaggaatagaatagaaagtaactgttctttttttttacagtagtgtttgagttagtgatcctaaaatggattaaagattaagttactttagggataacagcgtaat

SEQ ID NO: 759 - DNA, 199 bp, Cambarus maculatus
  source 1..199: mol_type = genomic DNA
aagggacgataagaccctataaaactttatattgagaggtgagaggtaattttgtttaaaagttattgctttaaagtatttggttggggcgacaaggatacaaggtaaagtaactgttttttttttctacaataatgtttgagtgaatgatcctaagatggattaaaagattaagttactttagggataacagcgtaat

SEQ ID NO: 760 - DNA, 200 bp, Cambarus coosawattae
  source 1..200: mol_type = genomic DNA
aagggacgataagaccctataaaactttatattgagaagtaatgggcaggttttgtttagaagttattatttttagagtatttggttggggcgacaaggataaaaagaaagtaactgttcttttattttacaataatgtttgggtataatgatcctaatatggattaaagattaagttactttagggataacagcgtaat

SEQ ID NO: 761 - DNA, 201 bp, Cambarus latimanus
  source 1..201: mol_type = genomic DNA
gaagggacgataagaccctataaaactttatattaaaaagtgatgtgtaagttttgtttaaaggttattactttaaagtatttggttggggtgacaggaatagaatagaaagtaactgttcttttttttttacagtagtgtttgagttagtgatcctaaaatggattaaagattaagttactttagggataacagcgtaat

SEQ ID NO: 762 - DNA, 200 bp, Cambarus strigosus
  source 1..200: mol_type = genomic DNA
aagggacgataagaccctataaaactttatattgaaaagtgataggtacaatttgtttgaagggttgttattttaaagtatttggttggggtgacaaggataaaatggaaagtaactgttttttttttttacagtgatgtttgggttaatgatcctaaaatggattaaagattaagttactttagggataacagcgtaat

SEQ ID NO: 763 - DNA, 198 bp, Cambarus parrishi
  source 1..198: mol_type = genomic DNA
aagggacgataagaccctataaaactttatactgagaggtgatggatgagttttgtttagaagttattattttagagtatttggttggggcgacaagggtaaaaagaaagtaactgttctttatttttacagttatgtttgagtgaatgatcctaaaacgggttaaagattaagttactttagggataacagcgtaat

SEQ ID NO: 764 - DNA, 200 bp, Cambarus bouchardi
  source 1..200: mol_type = genomic DNA
aagggacgataagaccctataaaactttatatttagaagtgataggtaagttttgtttgtagtttaattactttaaaatatttggttggggtgacaaggatagaataacaagtaactgtttttttttttaacaatgatgtttggatcggtgatcctaaaaggattaaaagattaagttactttagggataacagcgtaat

SEQ ID NO: 765 - DNA, 200 bp, Cambarus fasciatus
  source 1..200: mol_type = genomic DNA
aagggacgataagaccctataaaactttatattaagaagtaatgggtgaattttgtttagaagttattattttaaaatatttggttggggcgacaaggataaaaagaaaataactgttcttttattttacaataatgtttgggtataatgaccctaataggggttaaaagattaagttactttagggataacagcgtaat

SEQ ID NO: 766 - DNA, 200 bp, Cambarus harti
  source 1..200: mol_type = genomic DNA
aagggacgataagaccctataaaactttatattgaaaagtggtaggtatggtttgtttaaaaggttattgctttaaagtatttggttggggtgacaaggatagaatggaaagtaactgttttttttttttacaatgatgtttgagctaatgatcctaaaatggattaaagattaagttactttagggataacagcgtaat

SEQ ID NO: 767 - DNA, 198 bp, Cambarus nerterius
  source 1..198: mol_type = genomic DNA
aagggacgataagaccctataaaactttatatttgggggtaataggtaggttttgtttgttagttattattttaaagtatttggttggggtgacaaggataaaaaaaaagtaactgttctttatttttacaataatgtttggtataatgatcctaatatggattaaagattaagttactttagggataacagcgtaat

SEQ ID NO: 768 - DNA, 196 bp, Cambarus setosus
  source 1..196: mol_type = genomic DNA
aagggacgataagaccctataaaactttatatttaggagtagagttagttttattttaatatcttattttaaagtatttggttggggtgacatgaacaaaaaatgaaataattgtttttattttttacaatgatatttggacttatgatcctaaagtggattaaagattaagttactttagggataacagcgtaat

SEQ ID NO: 769 - DNA, 201 bp, Cambarus batchi
  source 1..201: mol_type = genomic DNA
aggggacgataagaccctataaaactttatattgaaaagtggtaggtacgatttgttttaaggttactactttaaagtatttggttggggcgacaagaatagaatagaaagtagctgttttttttattttacaatgatgtttgggttagtgatcctaaaacggattaaaagattaagttactttagggataacagcgtaat

SEQ ID NO: 770 - DNA, 199 bp, Cambarus halli
  source 1..199: mol_type = genomic DNA
aagggacgataagaccctataaaactttatattgaaaagtggtgggtatggtttgtttaaagattattactttaaagtatttggttggggtgacaagaatagaatagaaattaactgttttttatttttacaatgatgtttgagttagtgatcctaaaatggattaaagattaagttactttagggataacagcgtaat

SEQ ID NO: 771 - DNA, 200 bp, Cambarus crinipes
  source 1..200: mol_type = genomic DNA
aagggacgataagaccctataaaactttatatttagaagtgataggtaagttttgtttgtagtttaattactttaaaatatttggttggggtgacaaggatagaataacaagtaactgtttttttttttaacaatgatgtttggatcggtgatcctgaaaggattaaaagattaagttactttagggataacagcgtaat

SEQ ID NO: 772 - DNA, 200 bp, Cambarus unestami
  source 1..200: mol_type = genomic DNA
aagggacgataagaccctataaaactttatatttagaagtggtgggtaagttttgtttgtagtttaattactttaaaatatttggttggggtgacaaggatagaatagcaagtaactgtttttttttaaaacaatgatgtttggattaatgatcctaaagggattaaaagattaagttactttagggataacagcgtaat

SEQ ID NO: 773 - DNA, 199 bp, Cambarus reburrus
  source 1..199: mol_type = genomic DNA
aagggacgataagaccctataaaactttatatttggaagtgatgggtaagttttgtttgccggttattattttaaagtatttggttggggtgacaaggataaaaaaaagtaactgttctttattttttacaataatgtttggtataatgatcctaatatggattaaaagattaagttactttagggataacagcgtaat

SEQ ID NO: 774 - DNA, 198 bp, Cambarus gentry
  source 1..198: mol_type = genomic DNA
agggacgataagaccctataaaactttatttgaaaagtgatgagtatggtttatttaaaaggttattattttaaagtatttggttggggcgacaagaacaaaacggaaagtaactggtttttttttttacaatgatgtttgagttaatgatcctaaaatggattaaagattaagttactttagggataacagcgtaat

SEQ ID NO: 775 - DNA, 199 bp, Cambarus hubbsi
  source 1..199: mol_type = genomic DNA
aagggacgataagaccctataaaactttatattaggaagtgataagtatattttgtttaaagaattattactttaaagtatttggttggggtgacaaaaataaaaaaaaagtaactgtttttttttttaacaataatgtttgggttagtgatcctaaaatggattaaagattaagttactttagggataacagcgtaat

SEQ ID NO: 776 - DNA, 198 bp, Cambarus friaufi
  source 1..198: mol_type = genomic DNA
aagggacgataagaccctataaaactttatattaaaaagtggtaggtatggtttgtttgaagattattatttttgagtatttggttggggcgacaggaataaaataaaagtaactgttttttttttttacaatgatgtttgagttaatgatcttaaaatggattaaagattaagttactttagggataacagcgtaat

SEQ ID NO: 777 - DNA, 200 bp, Cambarus obeyensis
  source 1..200: mol_type = genomic DNA
aagggacgataagaccctataaaactttatatttagaagtgataggtaagttttgtttgtagtttaattattttaaaatatttggttggggtgacaaggatataatagcaagtaactgtttttttttttaacaataatgtttgggttgatgatcctaaaaggattaaaagattaagttactttagggataacagcgtaat

SEQ ID NO: 778 - DNA, 200 bp, Cambarus cracens
  source 1..200: mol_type = genomic DNA
aagggacgataagaccctataaaactttatactgaaaagtggtgggtatggtttgttttaaagattattactttaaagtatttggttggggtgacaagaataaaatagaaactaactgttttttatttttacaatgatgtttgagttagtgatcctaaaacggattaaagattaagttactttagggataacagcgtaat

SEQ ID NO: 779 - DNA, 200 bp, Cambarus asperimanus
  source 1..200: mol_type = genomic DNA
aagggacgataagaccctataaaactttatatttaaaaggagtaagcaaattttatttagaagttattgcttgaaaatattttgttggggcgacaagaatagggaggtaaataactgttttttttttttgacaataatgtttggattagtgatcctgaaaggataataagattaagttactttagggataacagcgtaat

SEQ ID NO: 780 - DNA, 85 bp, Cambarus hobbsorum
  source 1..85: mol_type = genomic DNA
ctgttttttttttttacaataatgtttggtttagtgatcctaaaatggattaaagattaagttactttagggataacagcgtaat

SEQ ID NO: 781 - DNA, 199 bp, Cambarus williami
  source 1..199: mol_type = genomic DNA
aagggacgataagaccctgtaaaactttatatttaagagtggtgggtaaggtttgtttggaggtaattattttaaagtatttggttggggcgacaagaatagaaaaaaaaataactgtttttttttcttacaatgatgtttgagttaatgatcctaaaacggattaaagattaagttactttagggataacagcgtaat

SEQ ID NO: 782 - DNA, 198 bp, Cambarus howardi
  source 1..198: mol_type = genomic DNA
aagggacgataagaccctataaaactttatatttggaagtgatggtagattttgtttgttagttattattttaaagtatttggttggggcgacaaggataaaaagaaagtaactgttctttgtttttacaataatgtttggtttagtgatcctaatatggattaaaagattaagttactttagggataacagcgtaat

SEQ ID NO: 783 - DNA, 199 bp, Cambarus obstipus
  source 1..199: mol_type = genomic DNA
aagggacgataagaccctataaaactttatattaaaaagtaatgggtaagttttgtttaaaggttattactttaaagtatttggttggggtgacaggaatagaatagaaagtaactgttcttcttttttacagtagtgtttgagttagtgatcctaaaatggattaaagattaagttactttagggataacagcgtaat

SEQ ID NO: 784 - DNA, 199 bp, Cambarus girardianus
  source 1..199: mol_type = genomic DNA
aagggacgataagaccctataaaactttatattaagaagtaatgggtaaattttgtttagaagttattattttaaagtatttggttggggcgacaaggataaaaagaaaataactgttctttcattttacaataatgtttggagatagtgatcctaagatgggttaaagattaagttactttagggataacagcgtaat

SEQ ID NO: 785 - DNA, 199 bp, Cambarus cryptodytes
  source 1..199: mol_type = genomic DNA
gggggacgataagaccctataaaactttatattgaaagggggtaagtaaattttgtttaaggttatgtgcttaaaatatttggttggggtgacagggatagaagaaaaggtaactgtttttttttattacaaataatatttggattagtgatcctgaagtggatttaagattaagttactttagggataacagcgtaat

SEQ ID NO: 786 - DNA, 200 bp, Cambarus speciosus; identical to SEQ ID NO: 760
  source 1..200: mol_type = genomic DNA

SEQ ID NO: 787 - DNA, 198 bp, Cambarus sciotensis
  source 1..198: mol_type = genomic DNA
aagggacgataagaccctataaaactttatattaagaagtaatgggtaagttttgtttaaaagttattattttaaagtatttggttggggcgacaaggataaaaagagagtaactgttcttttgttttacaataatgtttgatattatgatcctaagaaggattaaagattaagttactttagggataacagcgtaat

SEQ ID NO: 788 - DNA, 199 bp, Cambarus georgiae
  source 1..199: mol_type = genomic DNA
aagggacgataagaccctataaaactttatattaagaagtaatgggtgaattttgtttggaagttattattttagagtatttggttggggtgacaaggataaaaagaaagtaactgttcttttattttacaataatgtttgggtttgatgatcctaagacggattaaagattaagttactttagggataacagcgtaat

SEQ ID NO: 789 - DNA, 201 bp, Cambarus pristinus
  source 1..201: mol_type = genomic DNA
aagggacgataagaccctataaaactttatattgagaagtgataggtaagttttgtttataggttaattactttaaaatatttggttggggcgaccgggatagaataacaagtaactgtctttttttaaaacaattgtgtttggattagtgatcctaagatggattaaaagattaagttactttagggataacagcgtaat

SEQ ID NO: 790 - DNA, 196 bp, Cambarus aculabrum
  source 1..196: mol_type = genomic DNA
aagggacgataagaccctataaaactttatatttaggagtagagttagttttattttgggatcttattttaaagtatttggttggggtgacatgaacaaaaaatgaaataattgtttttattttttacaatgatatttgggtttatgatcctaaagtggattaaagattaagttactttagggataacagcgtaat

SEQ ID NO: 791 - DNA, 199 bp, Cambarus englishi
  source 1..199: mol_type = genomic DNA
aagggacgataagaccctataaaactttatactaaaaagtgatgggtaagttttgtttaaaggttattactttaaagtatttggttggggtgacaggaatagaatagaaagtaattgttctttttttttacagtaatgtttgagttagtgatcctaaaatggattaaagattaagttactttagggataacagcgtaat

SEQ ID NO: 792 - DNA, 199 bp, Cambarus brachydactylus
  source 1..199: mol_type = genomic DNA
aagggacgataagaccctataaaactttatattgaaaaatggtaggtatggtttatctaaggatcattattttttagtatttggttggggcgacaaaaatagaataataagtaactgtttttttttttaacaatgatgtttgagttagtgatcctaaaatggattagagattaagttactttagggataacagcgtaat

SEQ ID NO: 793 - DNA, 198 bp, Cambarus cumberlandensis
  source 1..198: mol_type = genomic DNA
aagggacgataagaccctataaaactttatattaaaaagtagtgggtaaattttgtttagaaattattattttaaagtatttggttggggcgacagggataaaaggaaagtaactgttcttttattttacaataatgtttgggtacacgatcctaagatggattaaagattaagttactttagggataacagcgtaat

SEQ ID NO: 794 - DNA, 200 bp, Cambarus dubius
  source 1..200: mol_type = genomic DNA
aagggacgataagaccctataaaactttatattgaaagggggtaagtagattttgtttaaaagttatttcttagaaatatttggttggggcgacaagaataggaaggcaaataactgttttttttttttacaataatttttgggttaatgatcctgaagaggattaaaagattaagttactttagggataacagcgtaat

SEQ ID NO: 795 - DNA, 200 bp, Cambarus reflexus
  misc_feature 103: note = n is a, c, g, or t
  source 1..200: mol_type = genomic DNA
aagggacgataagaccctataaaactttatattgaaaagtgataggtataatttgtttaaaaggttattattttaaagtatttggttggggtgacaaggatanaatgaaaagtaactgttttttttttttacaatgatgtttgggttaatgatcctaaaatggattaaagattaagttactttagggataacagcgtaat

SEQ ID NO: 796 - DNA, 200 bp, Cambarus rusticiformis; identical to SEQ ID NO: 753
  source 1..200: mol_type = genomic DNA

SEQ ID NO: 797 - DNA, 201 bp, Cambarus scotti
  source 1..201: mol_type = genomic DNA
aagggacgataagaccctataaaactttatattaagaagtagtagggtgaattttgtttagaagatattattttaaagtatttggttggggcgacaaggataaaaagaaaataactgttcttttattttacaataatgtttgggtataatgatcctaagacgggttaaaagattaagttactttagggataacagcgtaat

SEQ ID NO: 798 - DNA, 200 bp, Cambarus coosae; identical to SEQ ID NO: 760
  source 1..200: mol_type = genomic DNA

SEQ ID NO: 799 - DNA, 200 bp, Cambarus distans; identical to SEQ ID NO: 771
  source 1..200: mol_type = genomic DNA

SEQ ID NO: 800 - DNA, 198 bp, Cambarus longirostris
  source 1..198: mol_type = genomic DNA
aagggacgataagaccctataaaactttatatttgggagtgatgggcaggttttgtttgttagttattattttaaagtatttggttggggtgacaaggataaaaagaaagtaactgttctttatttttacaataatgtttggtataatgatcctaatgtggattaaagattaagttactttagggataacagcgtaat

SEQ ID NO: 801 - DNA, 198 bp, Cambarus hubrichti
  source 1..198: mol_type = genomic DNA
aagggacgataagaccctataaaactttatattgagaagtgatgggtatatttgtttaaagattattactttaaagtatttggttggggtgacaagaatagaatagaaagttactgttttttttttttacaatgatatttgagttggtgatcctaaaagggattgaagattaagttactttagggataacagcgtaat

SEQ ID NO: 802 - DNA, 219 bp, Monomia lucida
  source 1..219: mol_type = genomic DNA
aggggacgataagaccctataaagctttataagtctaggaattttagatattttgtaggataaatttattttctgataacttatttggttggggcgacaaaggtataattatggataactgctattaattttgatacatttattggtgattgaaatttagggtaattgatcctctttggggattaaaagatcaagttactttagggataacagcgtaat

SEQ ID NO: 803 - DNA, 215 bp, Faughnia serenei
  source 1..215: mol_type = genomic DNA
gagggacgataagaccctataaagctttataatgtttttttttattataaattgtgcaattttagtttactaaaaagaaattattattttactggggcggtaaaagtatgttcttgaccaactgcttttgtttttaaaaacaagtatttttgaaaataaattgatccattattaatgattataagaacaagttactttagggataacagcgtaat

SEQ ID NO: 804 - DNA, 216 bp, Harpiosquilla melanoura
  source 1..216: mol_type = genomic DNA
gagggacgataagaccctataaagctttataacgtattttttttttataaactatttaattattaacttaatacaaaatgtacgttattttactggggcggtaaaagtataatcaagaataactgctttttttattaataacaattataattgaaaataaattgatccattattaatgatcataagaacaagttactttagggataacagcgtaat

SEQ ID NO: 805 - DNA, 216 bp, Harpiosquilla annandalei
  source 1..216: mol_type = genomic DNA
gagggacgataagaccctataaagctttatagtatattttttttttataaattattttgttattaacttattataaaatatatattattttactggggcggtaaaagtataattaagaataactgcttttctttttaataacagtgataactggaaataaattgatccattattaatgatcacaagaacaagttactttagggataacagcgtaat

SEQ ID NO: 806 - DNA, 205 bp, Cherax communis
  source 1..205: mol_type = genomic DNA
gagggacgataagaccctataaagtttatacatggagttaatcaagagtgatttggaatagtaaagttcattattagcaagatgtttagttggggtgactagaatataagttatataactgttttttgttttaatcagagatatctgtttatatggtccctttttaggattagagagtaaattactttagggataacagcgtaat

SEQ ID NO: 807 - DNA, 207 bp, Cherax cuspidatus
  source 1..207: mol_type = genomic DNA
gagggacgataagaccctgtaaagtttgtacatcagggttaatgggtagcgatttaggttaataaggctaactcttatggaggtgtttagttggggcgactgggatataagttatctaactgtttctttattggaatcaataatatttgagtttatggtcctctttaaggatttcagaataaattacttcagggataacagcgtaat

SEQ ID NO: 808 - DNA, 205 bp, Cherax paniaicus
  source 1..205: mol_type = genomic DNA
gagggacgataagaccctataaagtttatacatggagttaatcaagagtgatttggaatagtaaagttcattattagcaagatgtttagttggggtgactagaatataagttatataactgttttttgttttaatcagagatatctgtttatatgatccctttttaggattagagagtaaattactttagggataacagcgtaat

SEQ ID NO: 809 - DNA, 206 bp, Cherax lorentzi
  source 1..206: mol_type = genomic DNA
gagggacgataagaccctataaagtttatacatgaagctggttaagagtgatttagggtgataaagtttattattagcagggtgtttagttggggcgactgagatataaatcatataactgttttttgtttaaatcagagatacttgctggtattggtccctttttaggattagagaaaaaattactttagggataacagcgtaat

SEQ ID NO: 810 - DNA, 205 bp, Cherax albertisii
  source 1..205: mol_type = genomic DNA
gagggacgataagaccctataaagtttatacatgaagctggttaagagtgatttgaggtattaaagtttattatcagcagggtgtttagttggggcgactaggatataaattatataactgttttttgtttaaatcagagatacttgtttatatgatccctttttaggattagagaataaattactttagggataacagcgtaat

SEQ ID NO: 811 - DNA, 207 bp, Cherax rotundus
  source 1..207: mol_type = genomic DNA
gagggacgataagaccctataaagttttgacactaaattaattaaaggtgatttgggttataaagttttattattaaataagtgtttagttggggcgactaggatataagtgatttaactgtttcttcgtttaaaccaaaaatatttgggtttatgatccttttttaggatttttagagtaaattactttagggataacagcgtaat

SEQ ID NO: 812 - DNA, 207 bp, Cherax leckii
  source 1..207: mol_type = genomic DNA
gagggacgataagaccctgtaaagtttgtacatcagggttaatgggtagagatttaggttaataaggctaactcttatgggggtgtttagttggggcgactaggatataagttatctaactgtttctttgttggaatcaataatatttgagtttatgatcctctttaaggatttcagaataaattacttcagggataacagcgtaat

SEQ ID NO: 813 - DNA, 205 bp, Cherax murido
  source 1..205: mol_type = genomic DNA
gagggacgataagaccctataaagtttatacatggagttaatcaagagtgatttggaatagtaaagttcattattagcaagatgtttagttggggtgactagaatataagttatataactgttttttgttttaatcagagatatctgtttgtatggtccctttttaggattagagagtaaattactttagggataacagcgtaat

SEQ ID NO: 814 - DNA, 207 bp, Cherax wasselli
  source 1..207: mol_type = genomic DNA
gagggacgataagaccctataaagtttgtacagtgaattagtatgaaatagattagatgggtaaaatttgttactaaggagatgtttggttggggcgattagaatataggtgaagtaactgtttctttttttgagtcaaaaatttttgatttaatgacccttaataaggattatgaaagcaagttactttagggataacagcgtaat

SEQ ID NO: 815 - DNA, 207 bp, Cherax parvus
  source 1..207: mol_type = genomic DNA
gggggacgataagaccctataaagtttgtacattaggctaattaagagtgatttagatttataaggctttattattagggggtgttaagttggggcgactaggatataatttatataactgtttctttattataatcagaaatatttgagtttatgatccttttttaggatttttagagtaaattactttagggataacagcgtaat

SEQ ID NO: 816 - DNA, 205 bp, Cherax pallidus
  source 1..205: mol_type = genomic DNA
gagggacgataagaccctataaagtttatacatagagttaatcaagagtgatttggaatagtaaagttcattattagcaagatgtttagttggggtgactagaatataagttatataactgttttttgttttaatcagagatatctgtttatatggtccctttttaggattagagagtaaattactttagggataacagcgtaat

SEQ ID NO: 817 - DNA, 205 bp, Cherax cartalacoolah
  source 1..205: mol_type = genomic DNA
gagggacgataagaccctatgaagtttgtacattggattaattgggaatgatttatgtttagtaaggtttgctattgagaagatgtttagttggggcgactaggatataatatataactgtttcttggtaggagtcaaaaatatttgagttaatgatccttttttaggattttagagtaaattactctagggataacagcgtaat

SEQ ID NO: 818 - DNA, 205 bp, Cherax longipes; identical to SEQ ID NO: 806
  source 1..205: mol_type = genomic DNA

SEQ ID NO: 819 - DNA, 205 bp, Cherax rhynchotus
  source 1..205: mol_type = genomic DNA
gagggacgataagaccctataaagtttatacatggggctggttgagagtgattcagaatagtaaagtttattattagcaagatgtttagttggggcgactaagatataaattatataactgtcttttgtttaaatcagaggtgtctgttcgtatgatccttttttaggattggagagtaaattactttagggataacagcgtaat

SEQ ID NO: 820 - DNA, 207 bp, Cherax pulcher
  source 1..207: mol_type = genomic DNA
gagggacgataagaccctataaagtttatacatagggttagttaaaagtgatttaggatagtaaagtttgttactagcaagatgtttggttggggtgactagaatataagttatataactgttttttatttaagtcagaagtatttgtttatagtgatcccttatttaggattaaagagtaaattactttagggataacagcgtaat

SEQ ID NO: 821 - DNA, 205 bp, Cherax peknyi
  source 1..205: mol_type = genomic DNA
gagggacgataagaccctataaagtttatacacagaattaatcgaaaataatttggaataataaaatttcttattagtgaggtgtttggttggggcgactaggatataagttatataactgttttttgtttaaatcaaagatatttgtttatatgatccctttttaggattaaagagtaaattactttagggataacagcgttat

SEQ ID NO: 822 - DNA, 209 bp, Cherax setosus
  misc_feature 117..118: note = n is a, c, g, or t
  source 1..209: mol_type = genomic DNA
gagggacgataagaccctataaagtttgacattaaattaattaaaggcgatttaggtcgttaaagtcttattattatataagtgtttagttggggcgactaggatataagtgatttnnaactgttttcttcgttcgagtcaagaatatttgagtgtatgatccttttttaggattttagagtaaattactttagggataacagcgtaat

SEQ ID NO: 823 - DNA, 206 bp, Cherax misolicus
  source 1..206: mol_type = genomic DNA
gagggacgataagaccctataaagtttatacatggagttagttaagagtaatttagaatagtaaagtttattattaacaagatgtttggttggggcgactggaatataagttatataactgttttttatttaagtcagaaatatttgtttataatgatcctttttttggatcaaagagtaaattactttagggataacagcgtaat

SEQ ID NO: 824 - DNA, 207 bp, Cherax warsamsonicus
  source 1..207: mol_type = genomic DNA
gagggacgataagaccctataaagttttatacatggagttagttaagagtaatttagagtagtaaagtttattattagcaagatgtttggttggggcgactggaatataagttatataactgttttttatttaagtcagaaatatttgtttataatgatcccttttttggatttaagagtaaattactttagggataacagcgtaat

SEQ ID NO: 825 - DNA, 201 bp, Cherax solus
  source 1..201: mol_type = genomic DNA
gacgataagaccctataaagtttatacatggagttaatcaagagtgatttggaatagtaaagttcattattagcaagatgtttagttggggtgactagaatataagttatataactgttttttgttttaatcagagatatctgtttatatggtccctttttaggattagagagtaaattactttagggataacagcgtaat

SEQ ID NO: 826 - DNA, 205 bp, Cherax snowden
  source 1..205: mol_type = genomic DNA
gagggacgataagaccctataaagtttaaacactgagttagttaagagtgatttagagtagtaaggtttattattagttaggtgtttggttggggcgactagaatataagttatataactgttttttatttgaatcagagatatctgttcacatgatccctttttaggattaaagagtaaattactttagggataacagcgtaat

SEQ ID NO: 827 - DNA, 205 bp, Cherax buitendijkae; identical to SEQ ID NO: 806
  source 1..205: mol_type = genomic DNA

SEQ ID NO: 828 - DNA, 205 bp, Cherax boschmai
  source 1..205: mol_type = genomic DNA
gagggacgataagaccctataaagtttatacatggagttaatcaagagtgatttggaatagtaaagttcattattagcaagatgtttagttggggtgactagaatataagttatgtaactgttttttgttttaatcagagatatctgtttatatgatccctttttaggattagagagtaaattactttagggataacagcgtaat

SEQ ID NO: 829 - DNA, 205 bp, Cherax nucifraga
  source 1..205: mol_type = genomic DNA
gggggacgataagaccctataaagtttgtacatggggccgcttaggagcgatttagagaagtaaagtttattactagcaaggtgtttcgttggggcgactaagatataaattatataactgttttctgttagaatcagagatgtctgtacatatgatccctttttaggatgaaagagtaaattactttagggataacagcgtaat

SEQ ID NO: 830 - DNA, 205 bp, Cherax barrette
  source 1..205: mol_type = genomic DNA
gagggacgataagaccctataaagtttatacatggggctggttgagagtgattcagaatagtaaagtttattattagcaagatgtttagttggggcgactaagatataaattatataactgtcttttgtttaaaccagaggtatctgttcgtatgatccttttttaggattggagagtaaattactttagggataacagcgtaat

SEQ ID NO: 831 - DNA, 216 bp, Oratosquilla fabricii
  source 1..216: mol_type = genomic DNA
gagggacgataagaccctataaagctttatggtatttttcttttttataaattatgttattattaacttattttagaactaatattattttactggggcggtaaaagtataataaagactaactgcttttgttttgtataacaattataattgaaaataaattgatccattattaatgatcacaagaacaagttactttagggataacagcgtaat

SEQ ID NO: 832 - DNA, 203 bp, Astacopsis tricornis
  source 1..203: mol_type = genomic DNA
gggggacgataagaccctataaagtttaacataataatgataaaaaattaattaggttataaagtttattattacaaataatgttttgttggggcgacaagaataaaaataatttaactgttcttttttttatcaaaaatatttgggtgggtgatcttttttaaaagtattagagtaaattactttagggataacagcgtaat

SEQ ID NO: 833 - DNA, 210 bp, Thalamita admete
  source 1..210: mol_type = genomic DNA
gagggacgataagaccctataaagctttataagtttaggtattctatagatttccacataaaaatagattttcaaatgcttattttgttggggcgacaatggtacaataatagtaactgcttttaagtataacatatatggatgagtcatatttaattgatcctttctaaagattttaagatcaagttactttagggataacagcgttat

SEQ ID NO: 834 - DNA, 198 bp, Faxonius virilis
  source 1..198: mol_type = genomic DNA
aggggacgataagaccctataaaactttatattttaaggtggaagataattttattttaaaattactattttaaattattttgttggggcgacaaggatataagaaagggtaactgtcttttatttttacaataatatttgaattattgatcctaaaagggattaaagattaagttactttagggataacagcgtaat

SEQ ID NO: 835 - DNA, 142 bp, Thranita prymna
  source 1..142: mol_type = genomic DNA
aagggacgataagaccctataaagcatttggttggggcgacaatagtataattaataactgctttaaaataatacaaatatatttgaagttgatcctttataaagatctaagatcaagttactttagggataacagcgttat

SEQ ID NO: 836 - DNA, 204 bp, Astacopsis franklinii
  source 1..204: mol_type = genomic DNA
gggggacgataagaccctataaagtttaacataataaggataaaaaattaattaggtttataaagtttattattacaaataatgttttgttggggcgacaagaataaaagtaatttaactgttctttttttaatcaaaaatatttgggtgggtgatcttttctaaaagtattagagtaaattactttagggataacagcgtaat

SEQ ID NO: 837 - DNA, 203 bp, Cambaroides schrenckii
  source 1..203: mol_type = genomic DNA
gagggacgataagaccctataaaactttatatacttataatttaatagaaatctttgtttataaaaatttagattagagtattttattggggcgatgaaaataaaatcttaggtaactgtttttgttttttaacaataatatttgaattttagatcccgaaaaagatcaaaagattaagttactttagggataacagcgttat

SEQ ID NO: 838 - DNA, 199 bp, Orconectes australis
  source 1..199: mol_type = genomic DNA
gagggacgataagaccctataaaactttatattaaaaagtgatgagtgtgttttgtttaagggttattattttaaagtatttggttggggcgacaaaaatagaagggaaagtaactgtttttttttattacaataatgtttgagtggatgatcctaaagtggattaaagattaagttactttagggataacagcgtaat

SEQ ID NO: 839 - DNA, 168 bp, Thalamita chaptalii
  misc_feature 10: note = n is a, c, g, or t
  source 1..168: mol_type = genomic DNA
gctttataantttagggagatcttataaatatttaagtaacaataattttttaaatgtttattcggttggggcgacaagagtataacgaaaataactgctttaaattaagacagaaatatctgaatcatagttaattgatcctttttaaagattctaagatcaagtta

SEQ ID NO: 840 - DNA, 167 bp, Zygita longifrons
  source 1..167: mol_type = genomic DNA
gctttataaatttagagatcttatatatttttatatagctgtaatttcttaaatgtttatttggttggggcgacaatagtataacagaagtaactgcttttaggtagtacaagcttatttgagtaataccgttaattgatcctttataaagatttaaagaccaagtt

SEQ ID NO: 841 - DNA, 167 bp, Thalamita picta
  source 1..167: mol_type = genomic DNA
gctttataagtttaaagattttgtatattttattatagctataatttcttgaatacttatttaattggggcgataacagtataattatagtaactgcttgtaagtaatacaaaaatacttgaattgaaattataattgatcctttataaagattttaagatcaagtt

SEQ ID NO: 842 - DNA, 169 bp, Thalamita seurati
  misc_feature 139: note = n is a, c, g, or t
  source 1..169: mol_type = genomic DNA
gctttacaagtttaaagatcttatatatttttctatatcaataatttcttgattatttgtttggttggggcgacgatagtataataatagtaactgctttaaagtgtttcaaatgtatttgaatcgaaataataactgnatccttaaataaagatttaaagatcaagtt

SEQ ID NO: 843 - DNA, 164 bp, Thranita pelsarti
  source 1..164: mol_type = genomic DNA
gctttataagtttggagattttatatatttttctatagcaataatttcttgaaagcttatttggttggggcgacaatagtataattgaaataactgctttaaaataatacaaatatatttgaattataattagttgatcctttataaagatttaagatcaagtt

SEQ ID NO: 844 - DNA, 199 bp, Orconectes barri
  source 1..199: mol_type = genomic DNA
gagggacgataagaccctataaaactttatattgaaaagtgatgggtatgttttgtttagggattattattttaaagtatttggttggggcgacaaaaatagaagagaaagtaactgtttttttttattacaataatgtttgagttgatgatcctaaaatggattaaagattaagttactttagggataacagcgtaat

SEQ ID NO: 845 - DNA, 199 bp, Faxonius ronaldi
  source 1..199: mol_type = genomic DNA
atgggacgataagaccctataaaactttatatttcaaggtagaaaattaattttattttaaggttactattttaaaatattttgttggggcgacaagggtataagaaatgataactgtcttttatttttacaataatatttgatttattgatcctaaaagggattaaagattaagttactttagggataacagcgtaat

SEQ ID NO: 846 - DNA, 199 bp, Faxonius neglectus
  source 1..199: mol_type = genomic DNA
aggggacgataagaccctataaaactttatattttaaggtggaaattaattttattttgaggattactattttaaaatgtttcgttggggcgacaaggatataagaaagggtaactgtcttttggtttaacagtaatatttggtttgttgatcctaaaaaggattaaagattaagttactttagggataacagcgtgat

SEQ ID NO: 847 - DNA, 197 bp, Orconectes compressus
  source 1..197: mol_type = genomic DNA
aagggacgataagaccctataaaactttatattaaaaagtagaagttaattttatttgagtgttactatttaaaatatttggttggggcgatcggaataaaaggtctaataactgtcctttttttttacaataatatttggtttaatgatcctaaaagggatttaagattaagttactttagggataacagcgtaat

SEQ ID NO: 848 - DNA, 197 bp, Orconectes forceps
  source 1..197: mol_type = genomic DNA
aagggacgataagaccctataaaactttatattttgaggtagaaattaattttatttcatatcactattttaaagtatttcgttggggcgacaaggatacaagaaaaggtaactgtcttttgtttttacaaaaatatttggtttgttgatccttaaagggattaaagattaagttactttagggataacagcgtaat

SEQ ID NO: 849 - DNA, 200 bp, Orconectes pellucidus
  source 1..200: mol_type = genomic DNA
aagggacgataagaccctataaaactttatattgaaaagtggtgggtatgttttattttgaggttatcacttaaaagtatttggttggggtgacaaaaatagaaggaaaagtaactgtttttttttcttacagtgatgtttgggttagtgatcctaaaatggattaaaagattaagttactttagggataacagcgtaat

SEQ ID NO: 850 - DNA, 217 bp, Neoeriocheir leptognathus
  source 1..217: mol_type = genomic DNA
atgggacgataagaccctataaagcttaatattaaaattttatttgattaaattttttttcaataaaaatctaataattaaatttattttattggggcgataaaagtataatgtttgataactgcttaattttaattacacctatgcgtgaattaatttatgattgatcctaattaaagattaaaagtttaagttactttagggataacagcgttat

SEQ ID NO: 851 - DNA, 213 bp, Penaeus kerathurus
  source 1..213: mol_type = genomic DNA
gggggacgataagaccctataaagcttgacaataatcttattaaattataaattgttagtataacttgattttaattagagtttgtttcgttggggcgacgagaatataataaataactgttcttttaaatataattacaaaaataattggtaaaatattgatcctttattaaagattaaaagattaagttactttagggataacagcgtaat

SEQ ID NO: 852 - DNA, 213 bp, Penaeus marginatus
  source 1..213: mol_type = genomic DNA
gggggacgataagaccctataaagcttgacaataatcctgctaaattataaattgttagtataacttaattctagctagaatttgtttcgttggggcgacgggaatataataaataactgttcttttaaatatagttacagaaatgtctgctataagattgatcctttattaaagattaaaagattaagttactttagggataacagcgtaat

SEQ ID NO: 853 - DNA, 214 bp, Penaeus longistylus
  source 1..214: mol_type = genomic DNA
gggggacgataagaccctataaagcttgacaataacttcgttaaattataaattgttagtataacttgattttaactggggtttgtttcgttggggcgacgagaatataataaaataactgttcttttaaatataattacaaaaatgcttggtggataattgatcctttaataaagattaaaagattaagttactttagggataacagcgtaat

SEQ ID NO: 854 - DNA, 213 bp, Penaeus plebejus
  source 1..213: mol_type = genomic DNA
gggggacgataagaccctataaagcttgacaataactttgttaaattataaattgttagtataacttgattttaactagggtttgtttcgttggggcgacgagaatataataaataactgttcttttaaatataattacaaaaatgcttggtggatgattgatcctttattaaagattaaaagataaagttactttagggataacagcgtaat

SEQ ID NO: 855 - DNA, 215 bp, Metapenaeopsis liui
  source 1..215: mol_type = genomic DNA
agagggacgataagaccctataaagctttacaattatttagttaaattataaattgttggtataacttgatttaattaatatttgtttcgttggggcgacgagaataaaattaaaataactgttcttttattagtagttacaaatttgtttggttaaaaattgatcctttattagagatcaaaagattaagttactttagggataacagcgtaat

SEQ ID NO: 856 - DNA, 214 bp, Metapenaeopsis lamellata
  source 1..214: mol_type = genomic DNA
aagggacgataagaccctataaagctttacaattatttaattaaattataaattattggtttaacttaatttaattaatatttgtttcgttggggcgacgagaataaaattaaaataactgttcttttataattaattacaaatttgtttggttaaatattgatcctttattaaagattaaaagattaagttactttagggataacagcgtaat

SEQ ID NO: 857 - DNA, 214 bp, Metapenaeopsis acclivis
  source 1..214: mol_type = genomic DNA
aagggacgataagaccctataaagctttacaattatttagttaaattataaattgttggtttaacttgatttaattaatatttgtttcgttggggcgacgagaataaaatttaagtaactgttcttttataaatagtcacaaatttatttggttagaaattgatcctttcttaaagatcaaaagattaagttactttagggataacagcgtaat

SEQ ID NO: 858 - DNA, 212 bp, Metapenaeopsis commensalis
  source 1..212: mol_type = genomic DNA
aagggacgataagaccctataaagctttacaattatttggctaatttataaattgttggtttaacttaagtgagtcaatgtttgtttcgttggggcgacgagaataaaattaagtaactgttctttaaaagtaattacaaagttaattgataaataattggtcctttactaaagattaaaagattaagttactttagggataacagcgtaat

SEQ ID NO: 859 - DNA, 215 bp, Atypopenaeus stenodactylus
  source 1..215: mol_type = genomic DNA
gtgggacgataagaccctataaagctttacaattatttgattaaattataaattgttagtataaacttaatttagttaatatttgttttgttggggcgacgagaatataattttaataactgttcttttataataatttactttgataattggagaataattgatcctttactaaagattaaaagattaagttactttagggataacagcgtaat

SEQ ID NO: 860 - DNA, 212 bp, Aristeus antillensis
  source 1..212: mol_type = genomic DNA
aggggacgataagaccctataaagctttacgatttgtttattaaatcaaaaattgtaagtataaattttatttaatagacatttgtttcgttggggcgacgagaataaaatttagtaactgttcttttagagtataacaattataattgaaggaaaattgatcccttattaaggattaaaaatttaagttactttagggataacagcgtaat

SEQ ID NO: 861 - DNA, 212 bp, Solenocera vioscai
  source 1..212: mol_type = genomic DNA
gggggacgataagaccctataaagctttacaataagttggttgaattataaattgttagtttaacttaatttaattggtgtttgtttcgttggggcgacgagaatataataatataactgttcttttgaaaatttaacaaagttgattggataataattgatcctttaataaagattaaaagattaagttactttagggataacagcgtaat

SEQ ID NO: 862 - DNA, 214 bp, Trachysalambria curvirostris
  source 1..214: mol_type = genomic DNA
gggggacgataagaccctataaagctttacaattatttagttaaattataaattgttagtataaacttaatttaattgatgtttgtttcgttggggcgacgagaatataattttgtaactgttcttttatagtttgttacaattatttttggtaaaagattgatcctttattaaagattaaaagattaagttactttagggataacagcgtaat

SEQ ID NO: 863 - DNA, 215 bp, Penaeus chinensis
  source 1..215: mol_type = genomic DNA
aggggacgataagaccctataaagcttgacaataatttaattatactatcaattgttagtgtaacttggttttaattaatatttgttacgttggggcgacgggaatataacaagtaactgttcttaaatatttaataacaaatataattggaaaactagcatgatcctctattagtgattaaaagattaagttactttagggataacagcgtaat

SEQ ID NO: 864 - DNA, 213 bp, Penaeus canaliculatus
  source 1..213: mol_type = genomic DNA
gggggacgataagaccctataaagcttgacaataacttcgttatattataaattgttagtataacttgattttaacgggggtttgtttcgttggggcgacgggaatataataaataactgttcttttaaatataattacaaaaatgtttggtaaataattgatcctctattagagattaaaagattaagttactttagggataacagcgtaat

SEQ ID NO: 865 - DNA, 213 bp, Metapenaeopsis barbata
  source 1..213: mol_type = genomic DNA
aagggacgataagaccctataaagctttacaattatttaattaagttataaattgttggtttaacttaacttagttaatatttgtttcgttggggcgacgagaataaaattaagtaactgttcttttgtagttaattacaaatttgtttggtagaaaattgatcctttattaaagattaaaagattaagttactttagggataacagcgtaat

SEQ ID NO: 866 - DNA, 213 bp, Penaeus esculentus
  source 1..213: mol_type = genomic DNA
aggggacgataagaccctataaagcttgacagtaagttgattatattataaattgttagtataacttgattttagttagcgtctgttacgttggggcgacgagaatataataagtaactgttcttaagtatctaataacaactatagttggtaaataaatgatcctctattagggattagaagattaagttactttagggataacagcgtaat

SEQ ID NO: 867 - DNA, 212 bp, Heteropenaeus longimanus
  source 1..212: mol_type = genomic DNA
gggggacgatcagaccctattaagcttgactagtatttatttaaattacaaactgtcagtataacgtaatcttaaataaaagttgtttcgttggggcgacgggaatataaacaataactgttcttttgattatcttacaaaaataattggtaaataagtgatcctttattaaagattaaaagattaagttactttagggataacagcgtaat

SEQ ID NO: 868 - DNA, 216 bp, Atypopenaeus dearmatus
  source 1..216: mol_type = genomic DNA
gagtgacgataagaccctataaagctttacaattatttggttgaattataaattattagtattaacttaattcaattaatatttgtttcgttggggcgacgggaatataattttagtaactgttcttttataattaattacttttattattaggaaaataattgatcctttattaaagattaaaagattaagttactttagggataacagcgtaat

SEQ ID NO: 869 - DNA, 212 bp, Funchalia taaningi
  source 1..212: mol_type = genomic DNA
aggggacgataagaccctataaaacttaacaataatttaattaaattataaattgtaagtgtaacttaattttaattaatacttgtttcgttggggcgacgagaatataaaaaataactgttcttttaaatatttacatgtgtatagttggtaaaaattgatcctttattaaagattaaaagattaagttactttagggataacagcgtaat

SEQ ID NO: 870 - DNA, 213 bp, Xiphopenaeus kroyeri
  source 1..213: mol_type = genomic DNA
aggggacgataagaccctataaagctttacaaatatctagttaatttataaattgttatataaacttaggttaattagtgcttgtttcgttggggcgacgagaatataattttgtaactgttcttttatggtttagtacaataatttttggtaaaaaattgatcctttattaaagattaaaagattaagttactttagggataacagcgtaat

SEQ ID NO: 871 - DNA, 213 bp, Trachypenaeopsis mobilispinis
  source 1..213: mol_type = genomic DNA
aagggacgataagaccctataaagctttacaaacacttagctggattttgaaatgttagtataaatttaattcagttaaagattgttccgttggggcgacgaaaatataaatttaagtaactgttttgaaaataaattacagatatatctgaaaataaatgatcctttaataaagattaagagatcaagttactttagggataacagcgtaat

SEQ ID NO: 872 - DNA, 217 bp, Rimapenaeus similis
  source 1..217: mol_type = genomic DNA
aggggacgataagaccctataaagctttacaattatcttgttaaattataaattgttagtttaatcttaatttaattagtgtttgtttcgttggggcgacgggaatataatgttttgtaactgttcttttataatttgttacaattaatgtttggtaaaaaattgatcctttattagagattaaaagattaagttactttagggataacagcgtaat

SEQ ID NO: 873 - DNA, 216 bp, Megokris granulosus
  source 1..216: mol_type = genomic DNA
gggggacgataagaccctataaagctttacaattatttaattagattataaactgttagtataaacttaatttaattaatacttgtttcgttggggcgacgagaatataattttggtaactgttcttttgtaaattattacaataaatttttggagaaaaaatgatcctttattagagattaaaagattaagttactttagggataacagcgtaat

SEQ ID NO: 874 - DNA, 215 bp, Parapenaeus politus
  source 1..215: mol_type = genomic DNA
gggggacgataagaccctataaagctttacaattatttaattagattataaattgttagtataacttaatttaattgatatttgtttcgttggggcgacgagaatataattttaagtaactgttcttttatggttaattacagatttatttgggtaaaaattgatcctttattagagattaaaagattaagttactttagggataacagcgtaat

SEQ ID NO: 875 - DNA, 210 bp, Solenocera membranacea
  source 1..210: mol_type = genomic DNA
gggggacgataagaccctataaagctttacaataatttaattaaattataaattgttagtataacttaatttaattaatatttgtttcgttggggcgacgggaataaaatagtataactgttcttttataaaattaaacagatttaattggataaagattgatcctttaataaagattaaaagattaagttactttagggataacagcga

SEQ ID NO: 876 - DNA, 214 bp, Solenocera koelbeli
  source 1..214: mol_type = genomic DNA
aggggacgataagaccctataaagctttacaataagttaattaaattataaattgttagtataacttggtttagttattatttgtttcgttggggcgacgagaatataattatatataactgttcttttaagaagaaaacaaggttaattggattagaattgatcctttaataaagattaaaagattaagttactttagggataacagcgtaat

SEQ ID NO: 877 - DNA, 206 bp, Alcockpenaeopsis hungerfordii
  source 1..206: mol_type = genomic DNA
gggggacgataagaccctataaagctttacaattatttaattaaattgtaaattgtttatttaacatggtttagttaatgtttgtttcgttggggcgacgggaatataatttagtaactgttctttatgagagacaataatttttggtattatgatcctctattagagattaaaagattaagttactttagggataacagcgtaat

SEQ ID NO: 878 - DNA, 210 bp, Batepenaeopsis tenella
  source 1..210: mol_type = genomic DNA
gggtgacgataagaccctataaaactttacaattatttaattgaatgatgaattgttagtataaatttgattcaattaatgtttgtttcgttggggcgacgaaaatataattttataactgttttataattttgtaacaatgatttttggtgaagatgatcctttattagagattaaaagattaagttactttagggataacagcgtaat

SEQ ID NO: 879 - DNA, 158 bp, Pandalus platyceros
  source 1..158: mol_type = genomic DNA
ataaaattttacaagatggggatttttttttgaattaaggtttaaattctataagttcctgtttgttgtgttggggcggcaaagataaaaattagtaactgtcatttaattagaataattataattagtttagttgatcctttattagagattaaaag

SEQ ID NO: 880 - DNA, 215 bp, Metapenaeus moyebi
  source 1..215: mol_type = genomic DNA
gggggacgataagaccctataaagctttacagtgtattgattaaattataaactgttagtataaacttaatttagttaataactgtttcgttggggcgacgagaatataataaacagtaactgttctttttttataataacaataaatatttggtattaattgatcctttattaaagattaaaagattaagttactttagggataacagcgtaat

SEQ ID NO: 881 - DNA, 216 bp, Metapenaeus joyneri
  source 1..216: mol_type = genomic DNA
gggggacgataagaccctataaagctttacagtgtgttaattaaattttaaactgttagtattaacttaatttatttaataactgtttcgttggggcgacgaaaatataatgaatagtaactgtttttctttgtaaaataacaataaatttttggaagtaattgatccttttttaaagattaaaagattaagttactttagggataacagcgtaat

SEQ ID NO: 882 - DNA, 206 bp, Pandalus montagui
  source 1..206: mol_type = genomic DNA
gggggacgataagaccctataaaattttacaaaatcggggtttttttctgaattaaggtgtaaattttataaatccctgtttgttgtgttggggcggcaaagataaaaattagtaactgtcatttaattagaataattataattagtttaattgatcctttattagagattaaaagattaagttactttagggataacagcgtaat

SEQ ID NO: 883 - DNA, 208 bp, Heterocarpus parvispina
  source 1..208: mol_type = genomic DNA
gggggacgataagaccctataaaactttacaagcgaagatttgtattttttaaattaaagtataacgttatgtactgttctgtttgttatgttggggcgacaaagatataataagtaactgttaattttattgaatagtgataattagtttaattgatcctctcttagagattacaagattaagttactttagggataacagcgtaat

SEQ ID NO: 884 - DNA, 214 bp, Penaeus brasiliensis
  source 1..214: mol_type = genomic DNA
aggggacgataagaccctataaagcttgacaataaatcgtttatattataaactgttagtgtaacttgattttaaacggtgtttgttgcgttggggcgacgggaatataaaaggtaactgttcttataacatttaataacagaaatatctgaaaaattaatgatccttttttaaagatcataagattaagttactttagggataacagcgtaat

SEQ ID NO: 885 - DNA, 211 bp, Aristeus antennatus
  source 1..211: mol_type = genomic DNA
gggggacgataagaccctataaagctttacaataaccagaatgcattaagatttgttagtataatttggtctatttcgggtttgtttcgatggggcgacgagaatataagttagataactgttctttttaggtatacaatattaattggttaaaaattgatcctttattaaagattataagattaagttactttagggataacagcgtaat

SEQ ID NO: 886 - DNA, 208 bp, Heterocarpus laevigatus
  source 1..208: mol_type = genomic DNA
gggggacgataagaccctataaaactttacaggtaaatctgtgcttcttaaattaaagtttaacattggtcatcgtcttgcctgttgtgttggggcgacaaagatataatttaaataactgtcaattttattaaatagcgataattagattagttgatcctttattagagattataagattaagttactttagggataacagcgtaat

SEQ ID NO: 887 - DNA, 208 bp, Heterocarpus lepidus
  source 1..208: mol_type = genomic DNA
gggggacgataagaccctgtaaaactttacagacattgtttgtatttttgaattaaggtttaatatcaattgttattgttgtttgttatgttggggcgacaaagatataattagaataactgtcaattttattaaataatgataattagttaattgatcctttattagagattataagattaagttactttagggataacagcgtaat

SEQ ID NO: 888 - DNA, 208 bp, Heterocarpus gibbosus
  source 1..208: mol_type = genomic DNA
gggggacgataagaccctataaaactttacagacattgtttgtatttttgaattaaagtttaatatcaattgttattgttgtttgttatgttggggcgacaaagatataattggaataactgtcaattttattaaatagtaataattagttaattgatcctttattagagattataagattaagttactttagggataacagcgtaat

SEQ ID NO: 889 - DNA, 207 bp, Funchalia villosa
  source 1..207: mol_type = genomic DNA
aggggacgataagaccctataaaacttgacaataatttaattaaattataaattgtaagtataacttgatttaattgatacttgttccgttggggcgacgagaatataaaataactgttcttttaaatatttacatatatatttggtaaagattgatcctttattaaagattaaaagattaagttactttagggataacagcgtaat

SEQ ID NO: 890 - DNA, 211 bp, Hemipenaeus carpenter
  source 1..211: mol_type = genomic DNA
gggggacgataagaccctataaagctttacaataatctaaatattccaagatttgttagtctaatttgggttatttagagtttgtttcgttggggcgacgagaatataaattaaataactgttcttttcagtactacaatattaattgattaaaaattgatcctttattaaagattaaaagattaagttactttagggataacagcgtaat

SEQ ID NO: 891 - DNA, 215 bp, Mesopenaeus tropicalis
  source 1..215: mol_type = genomic DNA
aggggacgataagaccctataaagctttacaataaaattattaaattataaattgttagtctaacttagtttaataattgtttgtttcgttggggcgacgagaatataataatataactgttcttttaaaaattatttaacagatttaattggattataattgatcctttaataaagattaaaagattaagttactttagggataacagcgtaat

SEQ ID NO: 892 - DNA, 213 bp, Pelagopenaeus balboae
  source 1..213: mol_type = genomic DNA
aggggacgataagaccctataaaacttaacaatattttaactaaattataaattgtaaacgtaacttaattttagttaatacttgtttcgttggggcgacgagaatataaataataactgttcttttaaatataattcacatatatgtatggtaaaaattgatcctttattaaagattaaaagattaagttactttagggataacagcgtaat

SEQ ID NO: 893 - DNA, 213 bp, Penaeus hathor
  source 1..213: mol_type = genomic DNA
gggggacgataagaccctataaagcttgacaataacttagttaaattataaattgttagtataacttgattttagccagggtttgtttcgttggggcgacgagaatataatgaataactgttcttttaaatataattacaaagataattggtgagcgattgatcctctaatagagattaaaagataaagttactttagggataacagcgtaat

SEQ ID NO: 894 - DNA, 214 bp, Metapenaeopsis provocatoria
  source 1..214: mol_type = genomic DNA
aagggacgataagaccctataaagctttacaattatttaattagattataaattgttggtgtaacttgatttaattaatatttgtttcgttggggcgacgggaataaaattgaaataactgttcttttattagtagtcacagatttgtttgattaaaaattgatcctttattagagattaaaagattaagttactttagggataacagcgtaat

SEQ ID NO: 895 - DNA, 211 bp, Aristeus virilis
  source 1..211: mol_type = genomic DNA
gggggacgataagaccctataaagctttacaataaccaagatgcactaagatttgttagtataatttggtctatttggggtttgtttcgttggggcgacgagaatataaattaaataactgttctttttagatatacaatattaattggttaaaaattgatcctttattaaagattacaagattaagttactttagggataacagcgtaat

SEQ ID NO: 896 - DNA, 126 bp, Aristeus alcock
  source 1..126: mol_type = genomic DNA
gggggacgataagaccctataaagctttacaataactgaaatacattaagatttgttagtataatttggtttattttgggtttgtttcgttggggcgacgagaatataagttagataactgttctt

SEQ ID NO: 897 - DNA, 214 bp, Penaeus aztecus
  source 1..214: mol_type = genomic DNA
aggggacgataagaccctataaagcttgacaataagtcatttatattataaattattagtataacttgatattaagtggtgtttgttgcgttggggcgacgagaatataaaaggtaactgttcttataacatttaataacagaaatatctggaaatttaatgatccttttttaaagatcataagattaagttactttagggataacagcgtaat

SEQ ID NO: 898 - DNA, 208 bp, Heterocarpus abulbus
  source 1..208: mol_type = genomic DNA
gggggacgataagaccctataaaactttacagacattgtttgtatttttgaattaaggtttaatatcaattgttattgttgtttgttatgttggggcgacaaagatataattggaataactgtcaattttattaaatagtaataattagttaattgatcctttattagagattataagattaagttactttagggataacagcgtaat

SEQ ID NO: 899 - DNA, 214 bp, Penaeus setiferus
  source 1..214: mol_type = genomic DNA
aggggacgataagaccctataaagctttacaataaattacttatattataaactgttagtataacttgattttaggtaatatttgttgcgttggggcgacgagaatataatgagtaactgttcttaagttaattaataacataaatatatggaagatgaatgatcctcttttagagatcataagattaagttactttagggataacagcgtaat

SEQ ID NO: 900 - DNA, 210 bp, Cerataspis monstrosus
  source 1..210: mol_type = genomic DNA
gggggacgataagaccctataaagctttacaataactcaggtgtactaagatttgtttgtataatttggtttacctgagacttgtttcgttggggcgacgagaatataattaaataactgttcttttggagtttacagtattaattggttaaaaattgatcctttattaaagattaaaagattaagttactttagggataacagcgtaat

SEQ ID NO: 901 - DNA, 212 bp, Pleoticus robustus
  source 1..212: mol_type = genomic DNA
gggggacgataagaccctataaagctttataataatttaattaaattataaattgttagtataacttaatttaattagtatttatttcgttggggcgacgagaatataataatataactgttcttttatagattaaacaaatttaattggataaaaattgatccttttataaagattaaaagattaagttactttagggataacagcgtaat

SEQ ID NO: 902 - DNA, 213 bp, Aristaeopsis edwardsiana
  source 1..213: mol_type = genomic DNA
gggggacgataagaccctataaagctttacaataacttgaatatactaaaatttgttagtgtaacttaatatatttgagatttgtttcgttggggcgacgagaatataaataaataactgttcttttgcttaaatatacaataataattggttaaatattgatcctttattaaagattaaaagattaagttactttagggataacagcgtaat

SEQ ID NO: 903 - DNA, 212 bp, Solenocera necopina
  source 1..212: mol_type = genomic DNA
gagggacgataagaccctataaagctttacaataagataattaaattataaattgttagtttaacttaatttaattattatttgtttcgttggggcgacgagaatataatagtataactgttcttttgcaatttaaacaaaattaattggataaaaattgatcctttaataaagattaaaagattaagttactttagggataacagcgtaat

SEQ ID NO: 904 - DNA, 215 bp, Parapenaeus indicus
  source 1..215: mol_type = genomic DNA
gggggacgataagaccctataaagctttacaattatttaattaaattataaattgttagtataacttaatttagttaatatttgtttcgttggggcgacgagaatataattttaagtaactgttcttttatagttaattacaaatttatttgggtaaagattgatcctttattatagattaaaagattaagttactttagggataacagcgtaat

SEQ ID NO: 905 - DNA, 215 bp, Parapenaeus cayrei
  source 1..215: mol_type = genomic DNA
gggggacgataagaccctataaagctttacaattatttaattaaattataaattgttagtataacttaacttagttaatatttgtttcgttggggcgacgagaatataattttaagtaactgttcttttatagttaattacaaatttatttgggtaaagattgatcctttattatagattaaaagattaagttactttagggataacagcgtaat

SEQ ID NO: 906 - DNA, 215 bp, Parapenaeus fissurus
  source 1..215: mol_type = genomic DNA
gggggacgataagaccctataaagctttacaattatttaattaaattataaattgttagtataacttagtttagttgatatttgtttcgttggggcgacgagaatataattttaagtaactgttcttttatagttaattacaaatttatttgggtaaagattgatcctttattatagattaaaagattaagttactttagggataacagcgtaat

SEQ ID NO: 907 - DNA, 216 bp, Parapenaeus investigatoris
  source 1..216: mol_type = genomic DNA
gggggacgataagaccctataaagctttacaattatttaattggattataaattgttagtataacttaatttagttgatatttgtttcgttggggcgacgagaatataatttttaagtaactgttcttttgtagttaattacagatttatttgggtaaaaattgatcctttattagagattaaaagattaagttactttagggataacagcgtaat

SEQ ID NO: 908 - DNA, 215 bp, Parapenaeus fissuroides; identical to SEQ ID NO: 904
  source 1..215: mol_type = genomic DNA

SEQ ID NO: 909 - DNA, 215 bp, Parapenaeus australiensis
  source 1..215: mol_type = genomic DNA
gggggacgataagaccctataaagctttacaattatttaattaaattataaattgttagtataacttaatttagttgatatttgtttcgttggggcgacgagaatataattttaagtaactgttcttttatagttaattacaaatttatttgggtaaagattgatcctttattatagattaaaagattaagttactttagggataacagcgtaat

SEQ ID NO: 910 - DNA, 215 bp, Parapenaeus americanus
  source 1..215: mol_type = genomic DNA
gggggacgataagaccctataaagctttacaattatttaattaaattataaattgttagtataacttaatttaattaatatttgtttcgttggggcgacgagaatataattttaagtaactgttcttttgtggttaattacagatttatttgggtaaaaattgatcctttattagagattaaaagattaagttactttagggataacagcgtaat

SEQ ID NO: 911 - DNA, 215 bp, Parapenaeus ruberoculatus; identical to SEQ ID NO: 909
  source 1..215: mol_type = genomic DNA

SEQ ID NO: 912 - DNA, 208 bp, Heterocarpus ensifer; identical to SEQ ID NO: 883
  source 1..208: mol_type = genomic DNA

SEQ ID NO: 913 - DNA, 205 bp, Kishinouyepenaeopsis cornuta
  source 1..205: mol_type = genomic DNA
gggggacgataagaccctataaagctttacaattgtttaattagattgtaaattgtttatttaacataatttaattaatgcttgtttcgttggggcgacggaaatataattagtaactgttttttataaaaaacaattatttttggtaaaatgatcctctattagagattaaaagatcaagttactttagggataacagcgtaat

SEQ ID NO: 914 - DNA, 215 bp, Parapenaeus sextuberculatus; identical to SEQ ID NO: 904
  source 1..215: mol_type = genomic DNA

SEQ ID NO: 915 - DNA, 215 bp, Parapenaeus perezfarfantae
  source 1..215: mol_type = genomic DNA
gggggacgataagaccctataaagctttacaattatttaattagattataaattgttagtataacttaatttagttaatatttgtttcgttggggcgacgagaatataattttaagtaactgttcttttatagttaattacaaatttatttgggtaaaaattgatcctttattatagattaaaagattaagttactttagggataacagcgtaat

SEQ ID NO: 916 - DNA, 215 bp, Parapenaeus murrayi
  source 1..215: mol_type = genomic DNA
gggggacgataagaccctataaagctttacaattatttaattggattataaattgttagtataacttagtttagttgatatttgtttcgttggggcgacgagaatataatttttaagtaactgttcttttgtagttaattacagatttatttgggtaaaattgatcctttattagagattaaaagattaagttactttagggataacagcgtaat

SEQ ID NO: 917 - DNA, 214 bp, Parapenaeus longipes
  source 1..214: mol_type = genomic DNA
aggggacgataagaccctataaagctttacaattgtttagttggattataaattgttagtataacttaatttagttagtatttgtttcgttggggcgacgagaatataatttaagataactgttctttttaattaattacaaatttgtttgggagaagattgatcctttattaaagattataagattaagttactttagggataacagcgtaat

SEQ ID NO: 918 - DNA, 215 bp, Parapenaeus lanceolatus; identical to SEQ ID NO: 904
  source 1..215: mol_type = genomic DNA

SEQ ID NO: 919 - DNA, 215 bp, Parapenaeus kensleyi; identical to SEQ ID NO: 904
  source 1..215: mol_type = genomic DNA

SEQ ID NO: 920 - DNA, 208 bp, Heterocarpus chani
  source 1..208: mol_type = genomic DNA
gggggacgataagaccctataaaactttacagacattgtttgtatttttgaattaaggtttaatatcaattgttattgttgtttgttatgttggggcgacaaagatataattggaataactgtcaattttattaaatagtgataattagttaattgatcctttattagagattataagattaagttactttagggataacagcgtaat

SEQ ID NO: 921 - DNA, 207 bp, Heterocarpus woodmasoni
  source 1..207: mol_type = genomic DNA
aggggacgataagaccctataaaactttacagatataattatacttattaaaataaagtttaatagtatttattattttatctgttatgttggggcgacaaagatataataaagtaactgttagttttatttgataataataattagtttaattgatcctttattaaagattataagaataagttactttagggataacagcgtaat

SEQ ID NO: 922 - DNA, 208 bp, Heterocarpus sibogae
  source 1..208: mol_type = genomic DNA
gggggacgataagaccctataaaactttacaagcaaagatttgtattttttaaattaaagtataacgttatgtactgttctgtttgttatgttggggcgacaaagatataataagtaactgttaattttattgaataatgataattagtttaattgatcctctcttagagattacaagattaagttactttagggataacagcgtaat

SEQ ID NO: 923 - DNA, 207 bp, Heterocarpus dorsalis
  source 1..207: mol_type = genomic DNA
gagggacgataagaccctataaaactttacaaatatatttgtaatttttaaattttagtttaacatcaattactggtttgtttgttgtgttggggcgacaatgatataattaaataactgtcaattttattaaatagtgataattagtttaattgatcctttactagagattataagattaagttactttagggataacagcgtaat

SEQ ID NO: 924 - DNA, 214 bp, Metapenaeopsis andamanensis
  source 1..214: mol_type = genomic DNA
gagggacgataagaccctataaagctttacaattatttaattagattataaattgttggtataacttgatttaattaatatttgtttcgttggggcgacgagaataaaattaaaataactgttcttttattagtagttacaaatttgtttggttaaaaattgatcctttattagagatcaaaagattaagttactttagggataacagcgtaat

SEQ ID NO: 925 - DNA, 208 bp, Heterocarpus corona; identical to SEQ ID NO: 888
  source 1..208: mol_type = genomic DNA

SEQ ID NO: 926 - DNA, 214 bp, Metapenaeopsis coniger
  source 1..214: mol_type = genomic DNA
gagggacgataagaccctataaagctttacaattatttaattagattataaattgttggtataacttgatttaattaatatttgtttcgttggggcgacgagaataaaattaaaataactgttcttttattagtagttacagatttgtttggttaaaaattgatcctttattagagatcaaaagattaagttactttagggataacagcgtaat

SEQ ID NO: 927 - DNA, 206 bp, Macrobrachium idella
  source 1..206: mol_type = genomic DNA
aatgggacgataagaccctataaaacttaatataattttggcttaatttgtgattattagtgaaaagtagttttgccgggtttatatttcgttggggagatgtagatataatatgtaactgtctatataatttaataattataattagtatttgatccttctttgtggattagaagaataagttactttagggataacagcgtgat

SEQ ID NO: 928 - DNA, 214 bp, Trachysalambria brevisuturae
  source 1..214: mol_type = genomic DNA
gagggacgataagaccctataaagctttataactagctgattaaattataaattattagtataaacttagtttaattggtgcttattttgttggggcgacaagaacataaaattgtaactgttcttttatagtttatatcattaatgtttggtaaataattgatcctttgttaaagattaaaagattaagttactttagggataacagcgtaat

SEQ ID NO: 929 - DNA, 213 bp, Trachysalambria aspera
  source 1..213: mol_type = genomic DNA
gggggacgataagaccctataaagctttacaattatctagttgaattataaattattagtataaacttagtttaattagtgtttgtttcgttggggcgacgagaatataattttgtaactgttcttttatgatttgttacaatgatttttggaaaaaattgatcctttattaaagattaaaagattaagttactttagggataacagcgtaat

SEQ ID NO: 930 - DNA, 214 bp, Trachysalambria albicoma
  source 1..214: mol_type = genomic DNA
gggggacgataagaccctataaagctttacaattatttgattaacttataaattattagtataaacttaacttaatcaatgtttgtttcgttggggcgacgagaatataattttgtaactgttctttagtagtttgttacaataatttttggtaaaaaattgatcctttattaaagattaaaagattaagttactttagggataacagcgtaat

SEQ ID NO: 931 - DNA, 211 bp, Euphausia superba
  source 1..211: mol_type = genomic DNA
aagtgacgataagaccctataaaacttgataaataaaataaaaaaagtggagtagttgattaaaacttttattgtggaatttattttattggggcgataaaaatataaaaagtaactgtttttaaagttttaatgatttataatcagattgtgtgaatgatcctttaataaagattataagattaagttactttagggataacagcgtaat

SEQ ID NO: 932 - DNA, 213 bp, Solenocera hextii
  source 1..213: mol_type = genomic DNA
gggggacgataagaccctataaagctttacaataaattaattaaattataaattattagtttaacttaatttaattagtgtttgtttcgttggggcgacgagaatataaattatataactgttcttttaggtgtataacaagattaattggagaaatattgatcctttagtaaagattaaaagattaagttactttagggataacagcgtaat

SEQ ID NO: 933 - DNA, 212 bp, Hymenopenaeus equalis
  source 1..212: mol_type = genomic DNA
aagggacgataagaccctataaagctttacaataagttaattaaattataaattgttagtataacttaatttaattaatgtttgtttcgttggggcgacgggaatataattatataactgttcttttataaatttaacaaaattaattggataataattgatcctttaataaagattaaaagattaagttactttagggataacagcgtaat

SEQ ID NO: 934 - DNA, 217 bp, Rimapenaeus constrictus
  source 1..217: mol_type = genomic DNA
aggggacgataagaccctataaagctttacaattatctaattaaattataaattgttagtttaatcttaatttaattagtgtttgtttcgttggggcgacgagaatataatgttttgtaactgttcttttgtaatttgttacagttaatagttggtaaaagattgatcctttattaaagattaaaagattaagttactttagggataacagcgtaat

SEQ ID NO: 935 - DNA, 212 bp, Crangon crangon
  source 1..212: mol_type = genomic DNA
aatgggaagacaagaccctataaaactttacaaaatatattaataaataatttggtagtttaaaattatattaggttatttttgttgagctggggcggtttttataaaaaaattaactgtaagttaaattaaaatataaatatatagtttaataagatccttaggtttggagattaaaagactaagttactttagggataacagcataattt

SEQ ID NO: 936 - DNA, 216 bp, Trachypenaeus anchoralis
  source 1..216: mol_type = genomic DNA
gagggacgataagaccctataaagctttacaattatttaattagattataaattgttagtataaacttagtttaattaatgcttgtttcgttggggcgacgagaatataattttggtaactgttcttttgtaagctattacaataaatttttggagaaaaaatgatcctttattaaagattaaaagattaagttactttagggataacagcgtaat

SEQ ID NO: 937 - DNA, 216 bp, Megokris pescadoreensis; identical to SEQ ID NO: 873
  source 1..216: mol_type = genomic DNA

SEQ ID NO: 938 - DNA, 215 bp, Trachysalambria longipes
  source 1..215: mol_type = genomic DNA
gagggacgataagaccctataaagctttacaattatttaactaaattatgaactgttagtataaatttaatttaattaatgtttgtttcgttggggcgacgagaatataatttttgtaactgttcttttgtagtttgttgcaatagtttttggaaaaagattgatcctttattaaagattaaaagattaagttactttagggataacagcgtaat

SEQ ID NO: 939 - DNA, 214 bp, Trachysalambria starobogatovi
  source 1..214: mol_type = genomic DNA
gggggacgataagaccctataaagctttacaattatttggttaaattataaattgttagtataaacttaatttaattgatgtttgtttcgttggggcgacgagaatataatttagtaactgttcttttataatttgttacaatgatttttggaaaagaaatgatcctctattagagattaaaagattaagttactttagggataacagcgtaat

SEQ ID NO: 940 - DNA, 214 bp, Trachysalambria nansei
  source 1..214: mol_type = genomic DNA
gggggacgataagaccctataaagctttacaattatttggttaaattatgaattgttagtataaatttaatttaattgatgtttgtttcgttggggcgacgggaatataattttgtaactgttcttttataatttgttacaataatttttggtaaaagattgatcctttattaaagattaaaagattaagttactttagggataacagcgtaat

SEQ ID NO: 941 - DNA, 216 bp, Trachysalambria malaiana
  source 1..216: mol_type = genomic DNA
gagggacgataagaccctataaagctttacaattatttaattagattataaattgttagtataaacttaatttagttagtgtttgtttcgttggggcgacgagaatataattttggtaactgttcttttgtaaattattacaataaatttttggagaaaaaatgatcctttattagagattaaaagattaagttactttagggataacagcgtaat

SEQ ID NO: 942 - DNA, 214 bp, Trachysalambria dentata; identical to SEQ ID NO: 862
  source 1..214: mol_type = genomic DNA

SEQ ID NO: 943 - DNA, 213 bp, Trachysalambria parvispina
  source 1..213: mol_type = genomic DNA
gggggacgataagaccctataaagctttacaattatttagttggattataaattgttagtataaacttaatttaactggtgtttgtttcgttggggcgacgggaatataattttgtaactgttcttttataacttgttacaattatttttggtaaaaattgatcctttattaaagattaaaagattaagttactttagggataacagcgtaat

SEQ ID NO: 944 - DNA, 164 bp, Crangon uritai
  source 1..164: mol_type = genomic DNA
actttacataatatattaataaataatttggtagtttaaaattatgttaggttatttatgttgagctggggcggtttttataaaaaaaataactgtaagttaaattaaaatataaatatatagtttaataagatccttaaatttggagattggaagactaagtt

SEQ ID NO: 945 - DNA, 206 bp, Pandalus borealis
  source 1..206: mol_type = genomic DNA
gggggacgataagaccctataaaattttacaaaattgggatttttttttgaattaaggtttaaattctataagttcttatttgttttgttggggcggcaaagataaaaattagtaactgtcatttaattagaataattataattagtttaattgatcctttattagagattaaaagattaagttactttagggataacagcgtaat

SEQ ID NO: 946 - DNA, 214 bp, Metapenaeus monoceros
  source 1..214: mol_type = genomic DNA
gggggacgataagaccctataaagctttacggtttgttaattaaattataaattgttagtataaacttaatttagctaacagctgtttcgttggggcgacgaaaatataatagatagtaactgtttttcttttataataacaatgatttttggtagtaattgatcctttattaaagattaaaagattaagttactttagggataacagcgtaat

SEQ ID NO: 947 - DNA, 206 bp, Pandalus nipponensis
  source 1..206: mol_type = genomic DNA
gggggacgataagaccctataaaattttacagagtcgggatctttttttaaattagtgtttaaattctataggttcttgtttgttttgttggggcgacaaagataaaatttagtaactgtcatttaattagaataagtataattagtttagttgatcctttattagagattaaaagattaagttactttagggataacagcgtaat

SEQ ID NO: 948 - DNA, 212 bp, Hadropenaeus lucasii
  source 1..212: mol_type = genomic DNA
gagggacgataagaccctataaagctttacaataaaatggttaaattataaattattagtataacttaatttaattattgtttgtttcgttggggcgacgagaatataataatataactgttctttatgaaaataaacaaaattaattggataaagattgatcctttaataaagattaaaagattaagttactttagggataacagcgtaat

SEQ ID NO: 949 - DNA, 249 bp, Ganjampenaeopsis uncta
  source 1..249: mol_type = genomic DNA
ttaaaaagtttattatacccaggtaataagcagttatgatcttgaaacttaaaggatttggcggtaatttagtctagttagaggaacctgtcctgtaatcgataaaccacgcagtatcttactttatcttgaaaatttatcagtttatataccatcattattagataactttaaaaaggtagagaagttattgaaataattttaagttagtatattagatcaaggtgtagctaatgataaagtagagat

SEQ ID NO: 950 - DNA, 213 bp, Solenocera annectens
  source 1..213: mol_type = genomic DNA
gggggacgataagaccctataaagctttacaataatttaattaaattataaattattagtttaacttaatttaattaataattgtttcgttggggcgacgagaatataaattatataactgttcttttaaaaatttaacaaatttaattggatgaaaattgatcctttaataaagattaaaagattaagttactttagggataacagcgtaat

SEQ ID NO: 951 - DNA, 214 bp, Solenocera melantho
  source 1..214: mol_type = genomic DNA
aggggacgataagaccctataaagctttacaataagttaattaaattataaattattagtttaacttaatttaattagtgtttgtttcgttggggcgacgagaatataaattatataactgttcttttaaagtaaataacaagattaattggaggaagattgatcctttaataaagattaaaagattaagttactttagggataacagcgtaat

SEQ ID NO: 952 - DNA, 216 bp, Parapenaeopsis stylifera
  source 1..216: mol_type = genomic DNA
ttagagggacgataagaccctataaagctttacaattattaaattgagctgtaaattgtttgttataacgtggctaagttaatgtttgtttcgttggggcgacgggaatataattagtaactgttcttatatatatatatataacaataatttttggaaaaatgatcctctaatagagattaaaagattaagttactttagggataacagcgtaat

SEQ ID NO: 953 - DNA, 213 bp, Penaeus japonicus
  source 1..213: mol_type = genomic DNA
gggggacgataagaccctataaagcttgacaataatttcgttatattataaattgttagtataacttgattttaacgggagtttgtttcgttggggcgacgggaatataataaataactgttcttttaaatataattacaaaaatacttggtaaataattgatcctctattagagattaaaagattaagttactttagggataacagcgtaat

SEQ ID NO: 954 - DNA, 214 bp, Penaeus brevirostris
  source 1..214: mol_type = genomic DNA
aggggacgataagaccctataaagcttgacaataagttgtttatattataaattgttagtataacttgattttaagcggtgtttgttacgttggggcgacgagaatataaaaggtaactgttcttatattgtttagtgacagatatatttggagagttaatgatcctttcttaaagatcacaagattaagttactttagggataacagcgtaat

SEQ ID NO: 955 - DNA, 214 bp, Penaeus notialis
  source 1..214: mol_type = genomic DNA
gggggacgataagaccctataaagcttgacaataagccgtttatattataaattgttagtataacttgattttaaatggtgtttgttgcgttggggcgacgagaatataaaagataactgttcttataatatttaataacagaaatagttggaaaattaatgatcctttctttaagatcataagattaagttactttagggataacagcgtaat

SEQ ID NO: 956 - DNA, 214 bp, Penaeus duorarum
  source 1..214: mol_type = genomic DNA
gggggacgataagaccctataaagcttgacaataagccgtttatattataaattgttagtataacttgattttaaatggtgtttgttgcgttggggcgacgagaatataaaagataactgttcttataatatttaataacagaaatagctggaaaattaatgatcctttctttaagatcataagattaagttactttagggataacagcgtaat

SEQ ID NO: 957 - DNA, 214 bp, Penaeus schmitti
  source 1..214: mol_type = genomic DNA
aggggacgataagaccctataaagctttacaataaattacttatattataaactgttagtataacttgattttaggtaatatttgttgcgttggggcgacgagaatataacgagtaactgttcttaagttaattaataacatagatatatggaagatgaatgatcctcttttagagatcataagattaagttactttagggataacagcgtaat

SEQ ID NO: 958 - DNA, 213 bp, Artemesia longinaris
  source 1..213: mol_type = genomic DNA
aagggacgataagaccctataaagctttacaattatttaattaaattgaaaattattagtataacttgatttagttaatagttgtttcgttggggcgacgagaatataattataaataactgttctttttacaaataacaaatttgtttggttaaaaattgatcctttattaaagattaaaagattaagttactttagggataacagcgtaat

SEQ ID NO: 959 - DNA, 214 bp, Penaeus subtilis
  source 1..214: mol_type = genomic DNA
aggggacgataagaccctataaagcttaacaataagttgtctatattataaattgttagtataacttgatatcagacggcgtttgttgcgttggggcgacgagaatataagaggtaactgtttttagattatttaataacagaaatatctggaaatttaatgatccttttttagagatcataagattaagttactttagggataacagcgtaat

SEQ ID NO: 960 - DNA, 214 bp, Penaeus stylirostris
  source 1..214: mol_type = genomic DNA
aggggacgataagaccctataaagctttacaataagttatctatattataaattgttagtataacttgattttagataatatttgttgcgttggggcgacgggaatataatgagtaactgttcttaagctatttaataacaggaatttctggaaaattaatgatcctcttttagagatcataagattaagttactttagggataacagcgtaat

SEQ ID NO: 961 - DNA, 214 bp, Penaeus vannamei
  source 1..214: mol_type = genomic DNA
aggggacgataagaccctataaagctttacaataagttacctatattataaattgttagtataacttgagtttaggtaacgtttgttgcgttggggcgacgagaatataataagtaactgttcttaagttatttaatgacagaaatttctggaaaattaatgatcctctactagagatcataagattaagttactttagggataacagcgtaat

SEQ ID NO: 962 - DNA, 204 bp, Macrobrachium rosenbergii
  source 1..204: mol_type = genomic DNA
gggggacgataagaccctataaaacttaatataatttaggcttaacttgcgatgtgggtgaaaagtagttttgtctggtttatatttcgttggggagatgaagatataatgagtaactgtctataaaattttatagcattgactagaatttgatccttccttggggattaggagaataagttactttagggataacagcgtgat

SEQ ID NO: 963 - DNA, 214 bp, Penaeus monodon
  source 1..214: mol_type = genomic DNA
aggggacgataagaccctataaaacttaacaataatttgattaaattataaattgttagtataacttgattttaattaatgtttgttgcgttggggcgacgggaatataattagtaactgttcttaaatattttattaacaagtataattgaagaataattgatcctttattaaagattaaaagattaagttactttagggataacagcgtaat

SEQ ID NO: 964 - DNA, 206 bp, Pandalus hypsinotus
  source 1..206: mol_type = genomic DNA
gggggacgataagaccctataaaattttacaaaattaggatttttttttaaattaaggtttaaattctataagttcttgtttgttgtgttggggcggcaaagataaaaattagtaactgtcatttaattagaataagtataattagtttagttgatcctttattagagattaaaagattaagttactttagggataacagcgtaat

SEQ ID NO: 965 - DNA, 210 bp, Ganjampenaeopsis uncta
  source 1..210: mol_type = genomic DNA
gggtgacgataagaccctataaaactttacaattatttaattgaatgatgaattgttagtataaatttgattcaattaatgtttgtttcgttggggcgacgaaaatataattttataactgttttataattttgtaacaatgatttttggtgaggatgatcctttattagagattaaaagattaagttactttagggataacagcgtaat

SEQ ID NO: 966 - DNA, 207 bp, Pandalus jordani
  source 1..207: mol_type = genomic DNA
gggggacgataagaccctataaaattttacaaaattgggattttttttttgaattaatgtttaaattttatgagttcctgtttgttttgttggggcggcaaagataaaaattagtaactgtcatttaattagaataattataattagtttaattgatcctttattagagattaaaagattaagttactttagggataacagcgtaat

SEQ ID NO: 967 - DNA, 206 bp, Macrobrachium bullatum
  source 1..206: mol_type = genomic DNA
atgggacgataagaccctataaaacttgatataaatttgacttaatttgcgattgtttgtgaaaagtagttttgctgtgtttatatttcgttggggagatgtagatataatatgtaactgtctgtttaatgtaataattataattagtttgtgatccttcatttgtggattagaagaataagttactttagggataacagcgtgat

SEQ ID NO: 968 - DNA, 214 bp, Penaeus penicillatus
  source 1..214: mol_type = genomic DNA
aggggacgataagaccctataaagcttgacaataatttaattatactatcaattgttagtataacttggttttaattaagatttgttgcgttggggcgacgagaatataataggtaactgttcttaaatatttaataacaaatataattgaaaattagtgtgatcctctattagcgattaaaagattaagttactttagggataacagcgtaat

SEQ ID NO: 969 - DNA, 214 bp, Penaeus merguiensis
  source 1..214: mol_type = genomic DNA
aggggacgataagaccctataaagcttgacaataatttaattatactatcaattgttagtgtaacttggttttaattaaaatttgttgcgttggggcgacgagaatataataggtaactgttcttaaatatttaataacaaatataattgaaaattagtgtgatcctctattagcgattaaaagattaagttactttagggataacagcgtaat

SEQ ID NO: 970 - DNA, 215 bp, Metapenaeus ensis
  source 1..215: mol_type = genomic DNA
gggggacgataagaccctataaagctttacagtgtgctaattaaattataaattgttagtataaacttggtttggttagtagctgtttcgttggggcgacgaaaatataattgatagtaactgtttttcttttataataacaataaatttttggtagaaattgatcctttattaaagattaaaagattaagttactttagggataacagcgtaat

SEQ ID NO: 971 - DNA, 113 bp, Acetes chinensis
  source 1..113: mol_type = genomic DNA
gagggacgataagaccctataaagctttactgtttaaattttttgatattagtattaaatgatcctctaataaagattaaaaatttaagttactttagggataacagcgtaat

SEQ ID NO: 972 - DNA, 218 bp, Macrobrachium nipponense
  source 1..218: mol_type = genomic DNA
agtgggacgataagaccctataaaactttatataagtttaatttggcttacaatttgagtgaaaagtagctttattaggcttatatttcgttggggagatgtagatgggataatcgactgtagaggcctgcatgctatttaatttaataattataattagtgtgtgatccttctttgtggattaaaagtataagttactttagggataacagcgtgat

SEQ ID NO: 973 - DNA, 214 bp, Penaeus californiensis
  source 1..214: mol_type = genomic DNA
aggggacgataagaccctataaagcttgacaataagtcgtttatattataaattgttagtgtaacttgattttaaacggtgtttgttgcgttggggcgacgggaatataaaaggtaactgttcttataatatttaataacagaaatatttggaaaattaatgatcctttcttaaagatcataagattaagttactttagggataacagcgtaat

SEQ ID NO: 974 - DNA, 204 bp, Macrobrachium lanchesteri
  source 1..204: mol_type = genomic DNA
atgggacgataagaccctataaaacttaatataatctagttaatacttgcgatttaggtaaaaaggagttttgtttagattatatttcgttggggagatgaagatataatgtataactgtctattgaataatataatgctaattagagtttgatccttctttgtggattaaaagaataagttactttagggataacagcgtaat

SEQ ID NO: 975 - DNA, 212 bp, Pleoticus muelleri
  source 1..212: mol_type = genomic DNA
aggggacgataagaccctataaagctttacaataaattaattaaattataaattataagtttaacttaatttaattagtatttgtttcgttggggcgacgagaatataattatataactgttcttttattgttataacaaatttaattgggtgaaaattgatccttttataaagattaaaagattaagttactttagggataacagcgtaat

SEQ ID NO: 976 - DNA, 214 bp, Metapenaeus affinis
  source 1..214: mol_type = genomic DNA
gggggacgataagaccctataaagctttacagtttgttgattaaattataaattgttagtgtgaacttaatttatttgatagctgtttcgttggggcgacgaaaatataataaatagtaactgtttttcttttataataacaatgatttttggtagtaattgatcctttattaaagattaaaagattaagttactttagggataacagcgtaat

SEQ ID NO: 977 - DNA, 212 bp, Hymenopenaeus neptunus
  source 1..212: mol_type = genomic DNA
aagggacgataagaccctataaagctttacaataagaaaattaaactataaattattagtataacttaatttaattggtatttgtttcgttggggcgacgggaatataataatataactgttcttttataagtttaacaaaattaattggataaaaattgatcctttaataaagattaaaagattaagttactttagggataacagcgtaat

SEQ ID NO: 978 - DNA, 214 bp, Penaeus indicus
  source 1..214: mol_type = genomic DNA
aggggacgataagaccctataaagcttgacaataatttaattatactatcaattgttagtataacttggttttaattaatatttgttgcgttggggcgacgagaatataataggtaactgttcttaaatatttaataacaaatataattggaaattagtatgatcctctattagcgattaaaagattaagttactttagggataacagcgtaat

SEQ ID NO: 979 - DNA, 211 bp, Aristaeomorpha foliacea
  source 1..211: mol_type = genomic DNA
aggggacgataagaccctataaagctttacaataaattaaatatattaaaatctgttagtgtaacttattatatttagagtttgtttcgttggggcgacgagaatataatgaataactgttctttttaaaaatagacaataataattggttaaagattgatcctttattaaagattaaaagattaagttactttagggataacagcgtaat

SEQ ID NO: 980 - DNA, 214 bp, Solenocera crassicornis; identical to SEQ ID NO: 876
  source 1..214: mol_type = genomic DNA

SEQ ID NO: 981 - DNA, 213 bp, Mierspenaeopsis hardwickii
  source 1..213: mol_type = genomic DNA
aggggacgataagaccctataaagctttacaataaattaattaaattataaattgttagtttaacttaatttaattagtgtttgtttcgttggggcgacgagaatataaattatataactgttcttttagataaataacaaggttaattggaggaagattgatcctttaataaagattaaaagattaagttactttagggataacagcgtaat

SEQ ID NO: 982 - DNA, 213 bp, Penaeus latisulcatus
  source 1..213: mol_type = genomic DNA
gggggacgataagaccctataaagcttgacaataacttcgttaaattgtaaattgttagtgtaacttgattttaactagggtttgtttcgttggggcgacgggaatataataaataactgttcttttaagtatagttacaaaaatgcttggtgaataattgatcctctactagagattaaaagataaagttactttagggataacagcgtaat

SEQ ID NO: 983 - DNA, 213 bp, Penaeus semisulcatus
  source 1..213: mol_type = genomic DNA
aggggacgataagaccctataaagcttgacaataagttaattatattataaattgttagtataacttgattttaattgacgtttgttacgttggggcgacgagaatataatgagtaactgttcttaaatgtttattgacaaatataattggtatttgattgatcctttattaaagattaaaagattaagttactttagggataacagcgtaat

SEQ ID NO: 984 - DNA, 214 bp, Penaeus silasi; identical to SEQ ID NO: 968
  source 1..214: mol_type = genomic DNA

SEQ ID NO: 985 - DNA, 214 bp, Penaeus isabelae
  source 1..214: mol_type = genomic DNA
aggggacgataagaccctataaagcttaacaataagctgtctatattataaactgttagtataacttgatactagatggtatttgttgcgttggggcgacgagaatataaaaggtaactgttcttataacatataataacagaaatatctggaaatttaatgatccttttttaaagatcataagattaagttactttagggataacagcgtaat

SEQ ID NO: 986 - DNA, 211 bp, Sicyonia lancifer
  source 1..211: mol_type = genomic DNA
gggggacgataagaccctataaagctttacaaaccctttttttagttaaaaattataagtgtaaactaacttaaaaattatttgtttcgttggggcgacgagaatataatttagtaactgttcttttaaaatattacagtattagttgattgaaaattgatcccttaataaggattaaaagtttaagttactttagggataacagcgtaat

SEQ ID NO: 987 - DNA, 213 bp, Metapenaeopsis dalei
  source 1..213: mol_type = genomic DNA
gagggacgataagaccctataaagctttacaattatttagttaagttataaattgttggtctaacttaatttaattaatatttgtttcgttggggcgacgagaataaaattaagtaactgttcttttgtaaataattacaaatctatttggtaaaaaattgatcctttattaaagattaaaagattaagttactttagggataacagcgtaat

SEQ ID NO: 988 - DNA, 214 bp, Metapenaeopsis gerardoi
  source 1..214: mol_type = genomic DNA
aagggacgataagaccctataaagctttacaattagtcaactaaatcataaattattggtttaacttggtttagttgatgtttgtttcgttggggcgacgagaatataattaaaataactgttctttaataattaattacagttttttctgatataaaattgatcctttattaaagattaaaagattaagttactttagggataacagcgtaat

SEQ ID NO: 989 - DNA, 215 bp, Parapenaeus longirostris
  source 1..215: mol_type = genomic DNA
gggggacgataagaccctataaagctttacaattatttaattaaattataaattgttagtataacttaatttaattgatatttgtttcgttggggcgacgagaatataattttaagtaactgttcttttatggttaattacagatttatttgggtaaaaattgatcctttattagagattaaaagattaagttactttagggataacagcgtaat

SEQ ID NO: 990 - DNA, 206 bp, Pandalus eous; identical to SEQ ID NO: 945
  source 1..206: mol_type = genomic DNA

SEQ ID NO: 991 - DNA, 205 bp, Pandalus miyakei
  source 1..205: mol_type = genomic DNA
gggggacgataagaccctataaaattttacaaaatcgggattttttttgaattaaggtttaagttctataattccttgtttgttgtgttggggcggcaaagataaaaattagtaactgtcatttaattagaataagtataattagtttagttgatcctttattagagattaaaagattaagttactttagggataacagcgtaat

SEQ ID NO: 992 - DNA, 206 bp, Pandalus japonicas
  source 1..206: mol_type = genomic DNA
gggggacgataagaccctataaaattttacaaaattggaatttttatttgaattagggtttaaattctatgatttcttgtttgttgtgttggggcgacaaagataaaagttagtaactgtcatttaagtagaataagtataattagtttagttgatcctttattagagattaaaagattaagttactttagggataacagcgtaat

SEQ ID NO: 993 - DNA, 205 bp, Pandalus glabrus
  source 1..205: mol_type = genomic DNA
gggggacgataagaccctataaaattttacaaaatcgggatcttttttgaattaaggtttaaattctatgattccttgtttgttgtgttggggcggcaaagataaaaattagtaactgtcatttaattagaataagtataattagtttagttgatcctttattagagattaaaagattaagttactttagggataacagcgtaat

SEQ ID NO: 994 - DNA, 207 bp, Pandalus teraoi
  source 1..207: mol_type = genomic DNA
gggggacgataagaccctataaaattttacagagttgggacctttattttaaattagtgtttaaatcttatgggttcttatttgttttgttggggcgacaaagataaaatttagtaactgtcatttaattagaataagtataattagtttagttgatcctttattagagattaaaagattaagttactttagggataacagcgtaat

SEQ ID NO: 995 - DNA, 206 bp, Pandalus ivanovi
  source 1..206: mol_type = genomic DNA
gggggacgataagaccctataaaattttacaaaattaggatttttttttaaattaaggtttaaattctataagttcttgtttgttgtgttggggcggcaaagataaaaattagtaactgtcatttaatcagaataagtataattagtttagttgatcctttattagagattaaaagattaagttactttagggataacagcgtaat

SEQ ID NO: 996 - DNA, 205 bp, Pandalus coccinatus
  source 1..205: mol_type = genomic DNA
gggggacgataagaccctataaaattttacaaaatcggggtttttttttaattaaggtttaaattctgtaatttcttgtttgttgtgttggggcggcaaagataaaaattagtaactgtcatttaattagaataagtataattagtttagttgatcctttattagagattaaaagatcaagttactttagggataacagcgtaat

SEQ ID NO: 997 - DNA, 205 bp, Pandalus formosanus
  source 1..205: mol_type = genomic DNA
gggggacgataagaccctataaaattttacagagtcgggtctttttttaaattagtgtttaaattctgcaggttcttatttgttttgttggggcgacaaagataaaatttagtaactgtcatttaatcagaatagatataattagtttagttgatcctttattagagattaaaagattaaattactttagggataacagcgtaat

SEQ ID NO: 998 - DNA, 205 bp, Pandalus princeps
  source 1..205: mol_type = genomic DNA
gggggacgataagaccctataaaattttacaaaattgggattttttttgaattaaggtttaaattctataattccttgtttgttgtgttggggcggcaaagataaaaattagtaactgtcatttaattagaataagtataattagtttagttgatcctttattagagattaaaagattaagttactttagggataacagcgtaat

SEQ ID NO: 999 - DNA, 206 bp, Pandalus chani
  source 1..206: mol_type = genomic DNA
gggggacgataagaccctataaaattttacagagtcgggatctttttttaaattagtgtttaaattctatgggttcttatttgttttgttggggcgacaaagataaaatttagtaactgtcatttaattagaataagtataattagtttagttgatcctttattagagattaaaagattaagttactttagggataacagcgtaat

SEQ ID NO: 1000 - DNA, 205 bp, Pandalus houyuu
  source 1..205: mol_type = genomic DNA
gggggacgataagaccctataaaattttacaaaatcggagttttttttgaattaaggtttaaattctttaattccttgtttgttgtgttggggcgacaaagataaaaattagtaactgtcatttaattagaataagtataattagtttagttgatcctttgttagagattaaaagattaagttactttagggataacagcgtaat

SEQ ID NO: 1001 - DNA, 205 bp, Pandalus capillus; identical to SEQ ID NO: 998
  source 1..205: mol_type = genomic DNA

SEQ ID NO: 1002 - DNA, 205 bp, Pandalus longirostris; identical to SEQ ID NO: 998
  source 1..205: mol_type = genomic DNA

SEQ ID NO: 1003 - DNA, 205 bp, Pandalus ochotensis; identical to SEQ ID NO: 1000
  source 1..205: mol_type = genomic DNA

SEQ ID NO: 1004 - DNA, 205 bp, Pandalus latirostris
  source 1..205: mol_type = genomic DNA
aggggacgataagaccctataaaattttacaaaatagggattttttttaaattaaggtttaaattctataagtctctgtttgttgtgttggggcggcaaagataaaaattagtaactgtcatttaattagaataagcataattagtttagttgatcctttattagagattaaaagattaagttagtttagggataacagcgtaat

SEQ ID NO: 1005 - DNA, 215 bp, Metapenaeus brevicornis
  source 1..215: mol_type = genomic DNA
gggggacgataagaccctataaagctttacagtgtattaattaaattttaaattgttagtattaacttagtttatttagtaactgtttcgttggggcgacgaaaatataataaatagtaactgtttttctttataatataacaaaaatttttggaagtaattgatcctttcttaaagattaaaagattaagttactttagggataacagcgtaat

SEQ ID NO: 1006 - DNA, 215 bp, Metapenaeus dobsoni; identical to SEQ ID NO: 1005
  source 1..215: mol_type = genomic DNA

SEQ ID NO: 1007 - DNA, 208 bp, Heterocarpus hayashii; identical to SEQ ID NO: 883
  source 1..208: mol_type = genomic DNA

SEQ ID NO: 1008 - DNA, 207 bp, Heterocarpus fascirostratus; identical to SEQ ID NO: 921
  source 1..207: mol_type = genomic DNA

SEQ ID NO: 1009 - DNA, 206 bp, Palaemon adspersus
  source 1..206: mol_type = genomic DNA
aggggacgataagaccctataaaactttataaatgaagtatgtaatctctaaattatattaaaatgttgataagtattttatttatttcgttggggcgacgttgatataatttgtaactgtctaaatgaataatataatcgtcattatactttgatccttctttgtggattataagactaagttactttagggataacagcgtaat

SEQ ID NO: 1010 - DNA, 206 bp, Palaemon serratus
  source 1..206: mol_type = genomic DNA
aggggacgataagaccctataaaactttatacatagaatataaaatctttaaattatattaaaatattgatggatattttatgtatttcgttggggagacgttgatataatttgtaactgtctgaatgaataatataattatcattatagtttgatccttctttgtggattataagaataagttactttagggataacagcgtaat

SEQ ID NO: 1011 - DNA, 209 bp, Palaemon sinensis
  source 1..209: mol_type = genomic DNA
aggggacgataagaccctataaaacttaatatttatttatattttgtttttaaattttaaataaactttacaaagtttagtatatattttgttggggagacattgacataattaataactgtctaatttaaatgtttatagctattgttattatatgatccttctttgaggatgaaaagagcaagttactttagggataacagcgtaat

SEQ ID NO: 1012 - DNA, 203 bp, Palaemon capensis
  source 1..203: mol_type = genomic DNA
aggggacgataagaccctataaaactttatataaattaagaattattgtgaattggacttaaatattttaatgcttaatttatatttcgttggggagacgctgatataatttgtaactgtcagttaaataaaatatttataattagtaaattgatctttcaatgagaatttaagagaagttactttagggataacagcgtaat

SEQ ID NO: 1013 - DNA, 206 bp, Palaemon annandalei
  source 1..206: mol_type = genomic DNA
gggggacgataagaccctataaaacttgataaaaagtttattttaactttaaattatatttaaaaattgtgaagttttctttttattttgttggggcgacattgatataaattgtaactgttttagcaaaaagtataattatcattatagtttgatccttctttggggataaaaagaataagttactttagggataacagcgtgat

SEQ ID NO: 1014 - DNA, 207 bp, Palaemon gravieri
  source 1..207: mol_type = genomic DNA
aagggacgataagaccctataaaactttataagagatttattttatttttaaattcttctttaaaaattgtgaagtatttctcttattttgttggggcgacattgatataagctgtaactgtttaaataaaaactataattatcattattgtttgatccttctttgtggataaaaagactaagttactttagggataacagcgtaat

SEQ ID NO: 1015 - DNA, 206 bp, Palaemon serenus
  source 1..206: mol_type = genomic DNA
gtgggacgataagaccctataaaactttatatgttgcctgctttttgttaaaattctacctaaaatttgaaaagcactctacatattttgttggggagacattgatataaattgtaactgtctaaataaaaaatataattatcattataatctgatccttctttgtggataaaaagaataagttactttagggataacagcgtgat

SEQ ID NO: 1016 - DNA, 205 bp, Palaemon carinicauda
  source 1..205: mol_type = genomic DNA
aggggacgataagaccctataaaacttaataaaaggttaattttaactatgaattgtatttaaaagttgtgaaattctctttttattttgttggggcgacattgatataagctgtaactgtcttgataataaaatagttatcactataagctgatccttttttagggataataagtataagttactttagggataacagcgtaat

SEQ ID NO: 1017 - DNA, 206 bp, Palaemon pugio
  source 1..206: mol_type = genomic DNA
aagggacgataagaccctataaaacttgacacgccactgtatttagctttaaattttaatctaaaattgtaagggtagctgtgtgttttgttggggagacattgatataagtagtaactgtctgatcaaaaattataattataattagcatatgatccctattttaggattataagaataagttactttagggataacagcgtaat

SEQ ID NO: 1018 - DNA, 201 bp, Palaemon pandaliformis
  source 1..201: mol_type = genomic DNA
aggggacgataagaccctataaaacttcacatgaaacgttgtagtttatggatcttgtttaatgtaatttcagcgttttttgttttgttggggcgacatggataaaaagtaactgtctattaaaattcaataacaataattagtttgtgatcctttattaaggattaaaagaataagttactttagggataacagcgtgat

SEQ ID NO: 1019 - DNA, 206 bp, Palaemon elegans
  source 1..206: mol_type = genomic DNA
aggggacgataagaccctataaaactttatgcacgacatgtcttgtctttaaattatatctaaatattggttggttaaaagtgcattccgttggggagacgttgatataagctgtaactgttttattaaatagaataattattattataatttgatccttctttgtggataaaaagaataagttactttagggataacagcgtaat

SEQ ID NO: 1020 - DNA, 206 bp, Palaemon longirostris
  source 1..206: mol_type = genomic DNA
attacgctgttatccctaaagtaacttattcttataacccacaaagaaggatcaaaatataatgatagttattttattcactcagacagttacaaattatatcaacgtctccccaacgaaatatattaagaatttatcagtattttaatataatttaaagattatatactttatatataaagttttatagggtcttatcgtccctc

SEQ ID NO: 1021 - DNA, 206 bp, Palaemon peringueyi
  source 1..206: mol_type = genomic DNA
gtgggacgataagaccctgtaaaactttatacgcatagtatttagcctttaaattatattaaaatattgatatatactttgggtatttcgttggggagacgttgatataatttgtaactgtctaattaaataaaataattatcattatactttgatccttctttgtgggttataagaataagttacttcagggataacagcgtaat

SEQ ID NO: 1022 - DNA, 207 bp, Palaemon debilis
  source 1..207: mol_type = genomic DNA
gggggacgataagaccctataaaactttatgtgatttgtggttatttattaaaattttacttaaaatttagaaaatctcatttacattttgttggggagacatagatataatttgtaactgtctgttgtaaaaatatagctatcgttagaatttgatccctctgtggggataataagaataagttactttagggataacagcgtaat

SEQ ID NO: 1023 - DNA, 206 bp, Palaemon carteri
  source 1..206: mol_type = genomic DNA
aggggacgataagaccctataaaactttatataaaataagtatatctacatattatgttaataggggtatgctattttatattttgttggggcgacatagatatgtaagcaaattaactgtttattaaaataaaataataataattagtttatgatcctttagtgaagattaaaagagtaagttactttagggataacagcgtgat

SEQ ID NO: 1024 - DNA, 205 bp, Palaemon ritteri
  source 1..205: mol_type = genomic DNA
aggggacgataagaccctataaaacttgatatagagctgactttagttttaaattttaaaataaaattgtaaagtaaactttatattttgttggggagacattgatataagtagtaactgtctgttaaaaattataattatatttagtttttgatccttctttgtggattagaagaataagttactttagggataacagcgtgat

SEQ ID NO: 1025 - DNA, 205 bp, Palaemon orientis; identical to SEQ ID NO: 1016
  source 1..205: mol_type = genomic DNA

SEQ ID NO: 1026 - DNA, 245 bp, Macrobrachium gracilirostre
  source 1..245: mol_type = genomic DNA
tgtgtaatttattacaaagtctagcctgcccactgatttaaggggacgataagaccctataaaacttaatataagttgggcttaatttatgatttaggtgaaaaatgattttgtttggtttatatttcgttggggagatgaagatataaaatgtaactgtctatttaaatttaataatattaattagtatgtgatccttctttgtggattataagaataagttactttagggataacagcgtaat

SEQ ID NO: 1027 - DNA, 206 bp, Palaemon vulgaris
  source 1..206: mol_type = genomic DNA
aagggacgataagaccctataaaacttaacacattgttatttctagctttgaattttaacctaaaattttagagataatcttgtgttttgttggggagacattgataaaactagtaactgtctaagtaaaaattataattataattagcatgtgatccctctatgaggattagaagaataagttactttagggataacagcgtaat

SEQ ID NO: 1028 - DNA, 206 bp, Palaemon serrifer
  source 1..206: mol_type = genomic DNA
gggggacgataagaccctataaaactttataaaagactaatttcatttttaaattctgttaaaaaattatgaaatttttcttttattttgttggggcgacattaatataagctgtaactgtttaaataaaaattataattatcattattgtttgatccctctttgaggataagaagactaagttactttagggataacagcgtaat

SEQ ID NO: 1029 - DNA, 207 bp, Palaemon varians
  source 1..207: mol_type = genomic DNA
gggggacgataagaccctataaaactttatacacgactcagttcatctttaaattttaattaaattaaagtgaaatgggacgtgtatttcgttggggagacgttgatataaactgtaactgtctaattgaaaaatataaaaataattattatatgatccttctttgtggataaaaagattaagttactttagggataacagcgtaat

SEQ ID NO: 1030 - DNA, 207 bp, Palaemon macrodactylus
  source 1..207: mol_type = genomic DNA
aagggacgataagaccctataaaactttataagagatttattttatttttaaattcttctttaaaaattgtgaagtaattcttttattttgttggggcgacattgatataagctgtaactgtttaaataaaaactataattatcattattgtttgatccttctttgtggataaaaagactaagttactttagggataacagcgtaat

SEQ ID NO: 1031 - DNA, 204 bp, Palaemon tonkinensis
  source 1..204: mol_type = genomic DNA
gggggacgataagaccctataaaactttatataaatataggttttttttaattttactaaaatattagagatccttatttatattttgttggggagacattgatataagcagtaactgtctgattaaaaataataatgatatttataaagtgaaccctcactgaggattaaaagaataagttactttagggataacagcgtaat

SEQ ID NO: 1032 - DNA, 206 bp, Palaemon xiphias
  source 1..206: mol_type = genomic DNA
cggggacgataagaccctataaaactttatacataaagtgtgtaatctctatattatattgaaatgtcgataagtactctatttatttcgttggggagaggtccatataccttgtaactgtctgagtgaacaaaataattatcattatattttgatccttctttgtggattataagagtaagttactttagggataacagcgtaat

SEQ ID NO: 1033 - DNA, 203 bp, Palaemon ivonicus
  source 1..203: mol_type = genomic DNA
tggggacgataagaccctataaaactttatataaaatatatttgtttataaattatgtttataagaaatttattattttatattttgttggggcgacataaataaaacatgtaactgtttatataaataaaataatgataattaaattttgatcctttaataaggattacaagaataagttactttagggataacagcgtaat

SEQ ID NO: 1034 - DNA, 205 bp, Palaemon pacificus
  source 1..205: mol_type = genomic DNA
gggggacgataagaccctgtaaaactttatacgtagggtatttagtctttaaattatactaaattgttgatatatatcttaggtatttcgttggggagacgtggatataatttgtaactgtctaattaataatataattatctttatattttgaaccttctttgtgggttataagaataagttacttcagggataacagcgtaat

SEQ ID NO: 1035 - DNA, 207 bp, Palaemon atrinubes
  source 1..207: mol_type = genomic DNA
gtgggacgataagaccctataaaactttatatgaattgagattatttgtcaaaattttaattaaaatttaaaagatcttatttatattttgttggggagacattgatataacttgtaactgtctagttgaaaaatatagttattgctagaatttgatccctctatgaggataaaaagaataagttactttagggataacagcgtgat

SEQ ID NO: 1036 - DNA, 206 bp, Palaemon intermedius
  source 1..206: mol_type = genomic DNA
gtgggacgataagaccctataaaactttacatgtggtacgcttttttttaaaattttaattaaactttggaaagtgttcttcatgttttgttggggagacattgatataatttgtaactgtctaaataaaacatataataatcattagaatttgatccttctttgtggataaaaagaataagttactttagggataacagcgtaat

SEQ ID NO: 1037 - DNA, 203 bp, Palaemon concinnus
  source 1..203: mol_type = genomic DNA
gggggacgataagaccctataaaactttacataagttacatttagtttgtgaataaagtaaaaaactgttttagataccttatgtttcgttggggagacgtttatataactgtaactgtcagataaatttaataattctaattagtatatgatccttctgtggggattataagagtaagttactttagggataacagcgtgat

SEQ ID NO: 1038 - DNA, 203 bp, Palaemon yuna
  source 1..203: mol_type = genomic DNA
gggggacgataagaccctataaaactttatataaaatatgtttgtttataaattgcgtttaaaggaaatttattgttttatattttgttggggcgacataaataaaatttgtaactgtttatataaataaaataacgataattagattttgatcttttaataaggattataagaataagttactttagggataacagcgtaat

SEQ ID NO: 1039 - DNA, 207 bp, Palaemon antennarius
  source 1..207: mol_type = genomic DNA
gggggacgataagaccctataaaactttatacgctgtttaatttgtctttgaattttaaataaattaaagtgaattagccagtgtatttcgttggggagacgttaatataaattgtaactgtttaattaaaaaatataaatataattatggtatgatccttctttgtggattaaaagattaagttactttagggataacagcgtaat

SEQ ID NO: 1040 - DNA, 206 bp, Palaemon dolospinus
  source 1..206: mol_type = genomic DNA
gtgggacgataagaccctataaaactttatatgtggcctgctttttcttaaaatttcagataaactttgaaaagcagttctcgtattttgttggggagacattgatataaatcgtaactgtctgaataaaatatatagtaatcactataatttgatcctcctatgcggctaaaaagaacaagttactttagggataacagcgtaat

SEQ ID NO: 1041 - DNA, 201 bp, Palaemon gracilis
  source 1..201: mol_type = genomic DNA
gggggacgataagaccctataaaactttacataaaaaactatagtctatgaattatatttaaggggagtctagtcttttgtgttttgttggggcgacatggataaaaagtaactgtctattaaaatttaataataataattagtttttgatcctttagtatggattaaaagaataagttactttagggataacagcgtgat

SEQ ID NO: 1042 - DNA, 206 bp, Palaemon mundusnovus
  source 1..206: mol_type = genomic DNA
aagggacgataagaccctataaaacttgatacgccgttatgcttagctttaaattttaatctaaaattgtaagggtggctgtgtgttctgttggggagacattgatataaatagtaactgtctgattaaaaaatataattataattagtatgtgatccttctttgtggattataagagtaagttactttagggataacagcgtaat

SEQ ID NO: 1043 - DNA, 206 bp, Palaemon suttkusi
  source 1..206: mol_type = genomic DNA
atgggacgataagaccctataaaactttatatgttatatattttatttttgaattattttaaaatattgtttaatgttttacatatttcgttggggagacgttgacataaaatgtaactgtttaaataaaaaatataattatcattatattttgatccttctttgtggataaaaagaataagttactttagggataacagcgtaat

SEQ ID NO: 1044 - DNA, 207 bp, Palaemon zariquieyi
  source 1..207: mol_type = genomic DNA
gggggacgataagaccctataaaactttatacattatttagttcgtctttaaattttaagtaaattatagtgaattgatatgtgtatttcgttggggagacgttgatataaattgtaactgtctaattaaaaaatataaatatgattatgatgcgatccttctttgtggataaaaagattaagttactttagggataacagcgtaat

SEQ ID NO: 1045 - DNA, 206 bp, Macrobrachium australiense
  source 1..206: mol_type = genomic DNA
gtgggacgataagaccctataaaactttatatgtcgtttgctttttcttaaaattttatgtaaactttgagaagcatttctcatattttgttggggagacattgatataagctgtaactgtctaaataaaatatataataatcattagaatttgatccttctttgtggataaaaagaataagttactttagggataacagcgtaat

SEQ ID NO: 1046 - DNA, 204 bp, Palaemon semmelinkii
  source 1..204: mol_type = genomic DNA
gggggacgataagaccctataaaactttatattttaattttttgctttagattatagattaaaattgcaaagttatttaaatatttcgttggggcgacgttgataaaatcttaactgtctgtgtaaaagttatagtaattactagaatatgatccttcttttgaggataacaagaataagttactttagggataacagcgtaat

SEQ ID NO: 1047 - DNA, 206 bp, Palaemon litoreus
  source 1..206: mol_type = genomic DNA
gtgggacgataagaccctataaaactttatatgttgcctgctttttgttaaaattctatctaaaatttgaaaagcactctacatattttgttggggagacattgatataaattgtaactgtctaaataaaaaatataattatcattataatttgatccttctttgtggataaaaagaataagttactttagggataacagcgtgat

SEQ ID NO: 1048 - DNA, 207 bp, Palaemon septemtrionalis
  source 1..207: mol_type = genomic DNA
aagggacgataagaccctataaaacttaatataaattatactatatttttaaattataaataatcatgataaagtttagtttatattttgttggggagatattgatataaattgtaactgtctaatttaaaaaaaataataatcattataatgtgatccttcattgaggataaaaagaataagttactttagggataacagcgtaat

SEQ ID NO: 1049 - DNA, 207 bp, Palaemon guangdongensis
  source 1..207: mol_type = genomic DNA
aagggacgataagaccctataaaactttataagagatttgttttatttttaaattcttctttaaaagttgtgaagtaattcttttattttgttggggcgacattgatataagctgtaactgtttaaataaaaactataattatcattattgtttgatccttctttgtggataaaaagactaagttactttagggataacagcgtaat

SEQ ID NO: 1050 - DNA, 202 bp, Palaemon hancocki
  source 1..202: mol_type = genomic DNA
gggggacgataagaccctataaaactttacattaaaaactatagtctataaattatatttaaggggagtctagcgtttatatgttttgttggggcgacatggataaaaagtaactgtctataaaaatttaataataataattagtttttgatcctttattatggattaaaagaataagttactttagggataacagcgtgat

SEQ ID NO: 1051 - DNA, 205 bp, Palaemon vietnamicus
  source 1..205: mol_type = genomic DNA
gggggacgataagaccctataaaacttaataaaaggtttattttaactataaattatatttaaaagttgtgaaatcttctttttattttgttggggcgacattgatataaactgtaactgtcttaataataaaataattatcattatgagctgatccttttttagggataataagtataagttactttagggataacagcgtaat

SEQ ID NO: 1052 - DNA, 206 bp, Palaemon texanus
  source 1..206: mol_type = genomic DNA
aagggacgataagaccctataaaacttgatacgccgttatgcttagctttaaattttaatctaaaattgtaaggatggctgtgtgttctgttggggagacattgatataaatagtaactgtctgattaaaaaatataattataattagtatgtgatccttctttgtggattataagagtaagttactttagggataacagcgtaat

SEQ ID NO: 1053 - DNA, 206 bp, Palaemon ortmanni
  source 1..206: mol_type = genomic DNA
aagggacgataagaccctataaaactttataagagacttattttatttttaaattctttttaaaaattataaaatatttcttttattttgttggggcgacattgatataacctgtaactgtttaaataaaaattataattatcattattgtttgatccttctttgtggataaaaagaataagttactttagggataacagcgtaat

SEQ ID NO: 1054 - DNA, 207 bp, Palaemon turcorum
  source 1..207: mol_type = genomic DNA
gggggacgataagaccctataaaactttatacgttaccctattcatctttgaagtttagataaattatagtgaatgggggggtgtatttcgttggggagacgttgatataatctgtaactgtctaattgaaaaatataaatataattagtacatgatccttctttgtggataaaaagattaagttactttagggataacagcgtaat

SEQ ID NO: 1055 - DNA, 206 bp, Palaemon kadiakensis
  source 1..206: mol_type = genomic DNA
aagggacgataagaccctataaaactttatatgtaaggtattttgtttttaaattattttgaaatattagttaatattttacatatttcgttggggagacgttgatataacatgtaactgtctaaataaaaaaaataattattattatattttgatccttctttgtggataaaaagaataagttactttagggataacagcgtaat

SEQ ID NO: 1056 - DNA, 204 bp, Macrobrachium asperulum
  source 1..204: mol_type = genomic DNA
gagggacgataagaccctataaaactttatatagacttaatttaacttgtgacttaagtgaaaaatagttttgttgagtttatattttgttggggagatgtagatataatatgtaactgtctatttaatttaataattataattagtgtttgatccttctttgtggattaaaagtgtaagttactttagggataacagcgtgat

SEQ ID NO: 1057 - DNA, 203 bp, Macrobrachium australe
  source 1..203: mol_type = genomic DNA
atgggacgataagaccctataaaacttaatataaatttagtttaatttgtgacataagtgaaaagtggttatacttcatttatatttcgttggggagatgtagatataaaagtaactgtctattaaatttaatagccatagttagtaattgatccttctttggggattaaaagagtaagttactttagggataacagcgtgat

SEQ ID NO: 1058 - DNA, 204 bp, Macrobrachium olfersii
  source 1..204: mol_type = genomic DNA
gtgggacgataagaccctataaaacttgatataagttgaacttgatttgtgatttaagtgaaaagcagttttgtttgacttatattttgctggggcggcatagatataaaatgtaactgtctattaaatttaatagtgctaattagtatttgatccttccttgtggattaaaagaataagttactttagggataacagcgtgat

SEQ ID NO: 1059 - DNA, 204 bp, Macrobrachium jelskii
  source 1..204: mol_type = genomic DNA
gagggacgataagaccctataaaacttaacataagttttgttttatttataattttagtgtaaaatggttttactaagtttatgtttcgttggggagatgaagatataaagagtaactgtcttttgaatagaataattataattagtttttgatccttctttggggattaagagaataagttactttagggataacagcgtgat

SEQ ID NO: 1060 - DNA, 204 bp, Macrobrachium villosimanus
  source 1..204: mol_type = genomic DNA
gggggacgataagaccctataaaactttatataatctaaacttgacttgggatttaagtgaaaagtagttttgttcggtttatatttcgttggggagatgaagatataatttgtaactgtctattgaatattatagtattaattagtatttgatccttctttgaggattaggagaataagttactttagggataacagcgtgat

SEQ ID NO: 1061 - DNA, 205 bp, Macrobrachium equidens
  source 1..205: mol_type = genomic DNA
gtgggacgataagaccctataaaacttaatataagttaaattggatttgtgatttcagtaaaaagtggtccttttaagtttatatttcgttggggagatgtagatataatgtgtaactgtttataaaactaaataatcataattagtttgtgatcctgcagatgtggattgaaagagtaagttactttagggataacagcgtaat

SEQ ID NO: 1062 - DNA, 205 bp, Macrobrachium potiuna
  source 1..205: mol_type = genomic DNA
gagggacgataagaccctataaaacttaatataaattgtaattttgtttatgatttaggtggaaaataatttaatttaatttatatttcgttggggagatgtagatataaaatgtaactgtctataaaatttaataattattgttagtggttgatccttctttggggattaaaagaataagttactttagggataacagcgtaat

SEQ ID NO: 1063 - DNA, 204 bp, Macrobrachium malcolmsonii
  source 1..204: mol_type = genomic DNA
gagggacgataagaccctataaaacttgatataatttgaacttgacttgtgatttgagtgaaaaatagtcttgtttggtttatatttcgttggggagatgaagatataatgtgtaactgtctattgaattttatagtattaattagaatttgatccttctttgaggattaggagaataagttactttagggataacagcgtgat

SEQ ID NO: 1064 - DNA, 219 bp, Macrobrachium superbum
  source 1..219: mol_type = genomic DNA
aggggacgataagaccctataaaactttatataaagttcatattcgattaaattttaatgttaataaaaattggatgttagctttatattttgttggggcgacaaaaataaaattgatagcaactgtttaaataattagacaaaaatatttgattatataattaaatgatcctaattaaagattaaaagtttaagttactttagggataacagcgttat

SEQ ID NO: 1065 - DNA, 204 bp, Macrobrachium striatum
  source 1..204: mol_type = genomic DNA
gtgggacgataagaccctataaaacttgatataagtttgattggatttgtgatttcagtaaaaaatggtcctctgaggcttatatttcgttggggagatgtagatataatatgtaactgtttgtaaaattaaataattataattagtttgtgatcctgccgtgtggattgaaagagtaagttactttagggataacagcgtaat

SEQ ID NO: 1066 - DNA, 204 bp, Macrobrachium latidactylus
  source 1..204: mol_type = genomic DNA
aagggacgataagaccctataaaacttgatacgagtttagcttaatttgtgatttatgtgaaaagtagttttgcttggttcgtatttcattggggagatgtagatataatctgtaactgtctatgaaatttaataatcataattagtagttgatccttctttgtggattaaaagagtaagttactttagggataacagcgtgat

SEQ ID NO: 1067 - DNA, 204 bp, Macrobrachium hancocki
  source 1..204: mol_type = genomic DNA
gtgggacgataagaccctataaaacttgatataagtgaaccttgatttgtgatttgtgtgaaaaatagtgttgctttacttatattttgctggggcggcatagatataaaatgtaactgtctgttaaatttaatagtgttaattagtttttgatccttccttgtggattagaagaataagttactttagggataacagcgtgat

SEQ ID NO: 1068 - DNA, 204 bp, Macrobrachium acanthurus
  source 1..204: mol_type = genomic DNA
gggggacgataagaccctataaaacttgatattgatctggcatgatttgcgattttagtgaaaagtagttttgtttgatctatatttcgttggggagatgaagatataaattgtaactgtctatttaatttaataattatagttagtgtttgatccttctttgtggattaagagattaagttactttagggataacagcgtgat

SEQ ID NO: 1069 - DNA, 204 bp, Macrobrachium inflatum
  source 1..204: mol_type = genomic DNA
gtgggacgataagaccctataaaactttatataagtttaatttggcttacaatttgagtgaaaagtagctttattaggtttatatttcgttggggagatgtagatataatgtgtaactgtctgtttaatttaataattataattagtgtgtgatccttctttgtggattaaaagtataagttactttagggataacagcgtgat

SEQ ID NO: 1070 - DNA, 204 bp, Macrobrachium crenulatum
  source 1..204: mol_type = genomic DNA
gtgggacgataagaccctataaaacttgatataagtgaagcttgatttgtgatttgggtgaaaaatagtgttgctttgcttatattttgctggggcggcatggatataaaatgtaactgtctgttaaatttaatagtgttaattagtttatgatccttctttgtggattaaaagaataagttactttagggataacagcgtgat

SEQ ID NO: 1071 - DNA, 204 bp, Macrobrachium carcinus
  source 1..204: mol_type = genomic DNA
gtgggacgataagaccctataaaactttatacaggttgggtcaaacttgtgatttgtgtgaaaagtagttttatttgatctgtattttgttggggcgacatagatataatgtgtaactgtctattaaattaaatagtgttaattagtctttgatccttctttgtggattagaagaataagttactttagggataacagcgtgat

SEQ ID NO: 1072 - DNA, 204 bp, Macrobrachium americanum
  source 1..204: mol_type = genomic DNA
gtgggacgataagaccctataaaactttatacaggttgggttcaatttgtgatttgtgtgaaaagtagttttattcggtctgtattttgttggggcgacatagatataatatgtaactgtctatcaaattaaatagtgttaattagtttttgatccttctttgtggattagaagaataagttactttagggataacagcgtgat

SEQ ID NO: 1073 - DNA, 204 bp, Macrobrachium latimanus
  source 1..204: mol_type = genomic DNA
gtgggacgataagaccctataaaacttgatatatgctgagtttaatttatgatataagtgaaaagtagttatacttggtttatatttcgttggggagatgtagatataaattgtaactgtctatttaatttaataatgttaattagtgtgtgatccctctttggggattaaaagaataagttactttagggataacagcgtgat

SEQ ID NO: 1074 - DNA, 205 bp, Macrobrachium mammillodactylus
  source 1..205: mol_type = genomic DNA
gtgggacgataagaccctataaaacttgatataagcttgatttaatttgcgattattagtgaaaagtagttttgttaggtttatattccgttggggagatgtagatataatgtgtaactgtctattaaatttaatagttataattagtttttgatccttctttgtggattagaagaataagttactttagggataacagcgtgat

SEQ ID NO: 1075 - DNA, 204 bp, Macrobrachium faustinum
  source 1..204: mol_type = genomic DNA
gtgggacgataagaccctataaaacttaatatgagttaagcttgatttgtgatctaagtgaaaaatagttttgtttgatttatattttgctggggcggcatagatataaaatgtaactgtctattaaatttaatagtgttaattagtctttgatccttctctgtggattaaaagaataagttactttagggataacagcgtgat

SEQ ID NO: 1076 - DNA, 204 bp, Macrobrachium heterochirus
  source 1..204: mol_type = genomic DNA
gtgggacgataagaccctataaaacttgatattatctagatttgatttgcgatttgagtgaagagcggttttatttatatgatattttgttggggagacatagatataattagtaactgtctattaaatttaatagtgttagttagcatttgatccttctttgtggattaaaagaataagttactttagggataacagcgtgat

SEQ ID NO: 1077 - DNA, 205 bp, Macrobrachium scabriculum
  source 1..205: mol_type = genomic DNA
gtgggacgataagaccctataaaacttgatataagtttagcttgatttgtgatttaggtgaaagacgattttgtttggtttatatttcgttggggagatgtagatataatgtgtaactgtctatttaatttaataaatataattagtatgtgatccttcatttgtggattggaagaataagttactttagggataacagcgtgat

SEQ ID NO: 1078 - DNA, 204 bp, Macrobrachium digueti
  source 1..204: mol_type = genomic DNA
gtgggacgataagaccctataaaacttgatataagctggacttgatttgtgatttaagtgaaaagcagttttgtttaacttatattttgctggggcggcatagatataaaatgtaactgtctgttaaatttaatagcgctaactagtatttgatccttctttgtggattaaaagaataagttactttagggataacagcgtgat

SEQ ID NO: 1079 - DNA, 204 bp, Macrobrachium tenellum
  source 1..204: mol_type = genomic DNA
gggggacgataagaccctataaaacttaatatcaatttggtttgatttgcgattttagtgaaaagtagtcgtgcttgactgatatttcgttggggagatgaagatataaattgtaactgtctatttaattcaatagctatggttagtgtgtgatccttctttgtggattaagagagtaagttactttagggataacagcgtgat

SEQ ID NO: 1080 - DNA, 205 bp, Macrobrachium idae
  source 1..205: mol_type = genomic DNA
gtgggacgataagaccctataaaacttaatataattttgatttgatttgcgattttcggtgaaaagtagtcttgtcgggtttatatttcgttggggagatgtagatataatatgtaactgtctatttaatttaatagctataactagtctttgatccttctttgtggattaaaagagtaagttactttagggataacagcgtgat

SEQ ID NO: 1081 - DNA, 213 bp, Macrobrachium japonicum; identical to SEQ ID NO: 864
  source 1..213: mol_type = genomic DNA

SEQ ID NO: 1082 - DNA, 204 bp, Macrobrachium formosense
  source 1..204: mol_type = genomic DNA
gtgggacgataagaccctataaaactttatataagtttaatttggcttacaatttgagtgaaaagtagctctattaagcttatgtttcgttggggagatgtagatataatgtgtaactgtctgtttaatttaataattataattagtgtgtgatccttctttgtggattaaaagtataagttactttagggataacagcgtgat

SEQ ID NO: 1083 - DNA, 207 bp, Macrobrachium dienbienphuense
  source 1..207: mol_type = genomic DNA
gtgggacgataagaccctataaaacttaatatgggtgtggcttgatttgcgattagtaggtgaaaagtaattttgtttggctcatattttgttggggagatgtggatataatgtgtaactgtctgttgaatttaatagttataattagtgtatgatccttcatttgtggattaggagaataagttactttagggataacagcgtgat

SEQ ID NO: 1084 - DNA, 205 bp, Macrobrachium placidulum
  source 1..205: mol_type = genomic DNA
gggggacgataagaccctataaaactttatataagtttggtttagtctatgatttaggtaaaaagtggttttgtctggtttatatttcgttggggagatgaggatataaaatgtaactgtctgtttaaatttaatagttttaattagtttgtgatccccctttggggattaaaagagtaagttactttagggataacagcgtgat

SEQ ID NO: 1085 - DNA, 204 bp, Macrobrachium sintangense
  source 1..204: mol_type = genomic DNA
gtgggacgataagaccctataaaactttatataagagtaacttggtttatgatttaggtgaaaagtagctttgttgggtttatatttcgttggggagatgtagatataatatataactgtctgtttaatttaatagctgtagttagtgtgtgatccttctttgtggattaaaagtataagttactttagggataacagcgtgat

SEQ ID NO: 1086 - DNA, 206 bp, Macrobrachium niphanae
  source 1..206: mol_type = genomic DNA
gtgggacgataagaccctataaaacttaatataggtttgacttaattttataaaatgggtgataaagtgattttgtttagtttatattttgttggggcgatatagatataatatgtaactgtctattaaatttaataattataattagtttttgatcctactttgtggattgaaagaataagttactttagggataacagcgtgat

SEQ ID NO: 1087 - DNA, 204 bp, Macrobrachium totonacum
  misc_feature 161: note = n is a, c, g, or t
  source 1..204: mol_type = genomic DNA
gtgggacgataagaccctataaaacttgatataagtttagtttaatttgtgatttgagtgaagagcaattttgttaaatttatattttgttggggagatgtagatataaaatataactgtctattgaatttaatagtaataattaatttttgatccttctntgaagattaggagaataagttactttagggataaccgcgctga

SEQ ID NO: 1088 - DNA, 204 bp, Macrobrachium tuxtlaense
  source 1..204: mol_type = genomic DNA
gtgggacgataagaccctataaaacttgatataagtttaatttaatttgtgatttaagtgaagaacaattttgttaaatttatattttgttggggagatgtagatataaaatataactgtctattaaatttaatagtaataattaatttttgatccttctgtggggattaggagaataagttactttagggataacagcgtgat

SEQ ID NO: 1089 - DNA, 204 bp, Macrobrachium vicconi
  source 1..204: mol_type = genomic DNA
atgggacgataagaccctataaaacttaatataaatttagtttaatttgtgatttaagtgaagagcagttttattaaatttatattttgttggggagatgtagatataaagtgtaactgtctaataaatttaataataataattaatttttgatccttccatgaggattaggagagtaagttactttagggataacagcgtgat

SEQ ID NO: 1090 - DNA, 204 bp, Macrobrachium villalobosi
  source 1..204: mol_type = genomic DNA
gtgggacgataagaccctataaaacttgatataagtttaatttaatttgtgatttaagtgaagagcgattttgttgaatttatattttgttggggagatgtagatataaagtgtaactgtctattgaatttaatagtaataattagtttttgatccttctgtggagattaagagagtaagttactttagggataacagcgtgat

SEQ ID NO: 1091 - DNA, 205 bp, Macrobrachium amazonicum
  source 1..205: mol_type = genomic DNA
gggggacgataagaccctataaaacttaatatctgtttagcttgatttgtgattttagtgaaaagtagttttgttaagttgatatttcgttggggagatgaagatataaattgtaactgtctattataatttgataattatagttagtgtgtgatccttctttgtggattaaaagaataagttactttagggataacagcgtaat

SEQ ID NO: 1092 - DNA, 205 bp, Macrobrachium canarae
  source 1..205: mol_type = genomic DNA
aagggacgataagaccctataaaacttgatatagttcaagcttgacttgtgattatgagtaaaaagtagttttgtttggtttatattttgttggggagataaagatataatgtgtaactgtctattgaattttatagtattaattagaatttgatccttctttgaggattaagagaataagttactttagggataacagcgtgat

SEQ ID NO: 1093 - DNA, 204 bp, Macrobrachium tratense
  source 1..204: mol_type = genomic DNA
gtgggacgataagaccctataaaactttatataaaagtaacttggtttatgatttaggtgaaaagtagctttgttgggtttatatttcgttggggagatgtagatataatatataactgtctgtttaatttaatagctgtagttagtgtgtgatccttctttgtggattaaaagtataagttactttagggataacagcgtgat

SEQ ID NO: 1094 - DNA, 207 bp, Macrobrachium forcipatum
  source 1..207: mol_type = genomic DNA
gtgggacgataagaccctataaaacttaatatgggtttggcttgatttgcgattagtgggtgaaaagtaattttgtttagctcatattttgttggggagatgtggatataatgtgtaactgtctgttgaatttaatagttatgattagtgtgtgatccttcatttgtggattaggagaataagttactttagggataacagcgtgat

SEQ ID NO: 1095 - DNA, 207 bp, Macrobrachium hirsutimanus
  source 1..207: mol_type = genomic DNA
gtgggacgataagaccctataaaacttaatataggtttagcttgatttgcaattatgggtgaaaagtggttttgtctaggcttatattttgttggggagatgtagatataatgtgtaactgtctgttgaatttaatagttatgattagtgtgtgatccttcatttgtggattaagagaataagttactttagggataacagcgtaat

SEQ ID NO: 1096 - DNA, 204 bp, Macrobrachium borellii
  source 1..204: mol_type = genomic DNA
gggggacgataagaccctataaaatttaatataagtttaattttatttgtaatttaagtgaaaaatagtttaattaaatttatatttcgttggggagatgtggatataaagtgtaactgtctataaaatttaatagtaattattagtaagtgatccttctttgtggatttgaagaataagttactttagggataacagcgtaat

SEQ ID NO: 1097 - DNA, 204 bp, Macrobrachium brasiliense
  source 1..204: mol_type = genomic DNA
gggggacgataagaccctataaaacttgatataaatttagttttactagtgatttaagtgaaaagtggtttaattaaagttatatttcgttggggagatgtaaatataaatgtaactgtttataaaattaaataattattgttagtaggtgatcctgcatatgcggattgagaaaataagttactttagggataacagcgtaat

SEQ ID NO: 1098 - DNA, 204 bp, Macrobrachium aemulum
  source 1..204: mol_type = genomic DNA
gtgggacgataagaccctataaaacttgatataaatttagtttgatttatgatgtgagtgaaaagttgttttgctaggtttatattccgttggggcgatgaagatataaaatgtaactgtctatttaatttaatagtattaattagtacttgatccttctttggggattaaaagaataagttactttagggataacagcgtgat

SEQ ID NO: 1099 - DNA, 205 bp, Macrobrachium handschini
  source 1..205: mol_type = genomic DNA
gtgggacgataagaccctataaaacttgatataagttttgcttgatttgtgatttgtagtgaaaagtggtttcgtttaatttatatttcgttggggagatgtagatataatatgtaactgtctatttaatttaatagttataattagtatttgatccttctttgtggattaagagaataagttactttagggataacagcgtgat

SEQ ID NO: 1100 - DNA, 204 bp, Macrobrachium horstii
  source 1..204: mol_type = genomic DNA
atgggacgataagaccctataaaacttaatataagttgatcttgatttgtgatttgggtgaaaaatgattttgcttggcttatatttcgttggggagatgaagatataaaatgtaactgtctgtgaaatttaatagtgttaattagtttttgatccttctttgtggattaagagaataagttactttagggataacagcgtgat

SEQ ID NO: 1101 - DNA, 205 bp, Macrobrachium ferreirai
  source 1..205: mol_type = genomic DNA
gggggacgataagaccctataaaacttaatataaatttgattttatttataattttagtgaaaagaagttttaataagtttatattttgttggggtgacgtggatataaagtgtaactgtccgtaaaatttaataattttaattagtgtctgatctttcttatggggattaaaagagtaagttactttagggataacagcgtgat

SEQ ID NO: 1102 - DNA, 205 bp, Macrobrachium lanatum
  source 1..205: mol_type = genomic DNA
gtgggacgataagaccctataaaacttgatatgagtttagcttgatttgtgatttaggtgaaagacgatcttgtttggtttatatttcgttggggagatgtagatataatgtgtaactgtctatttaatttaataaatatagttagtatgtgatccttcatttgtggattagaagaataagttactttagggataacagcgtgat

SEQ ID NO: 1103 - DNA, 205 bp, Macrobrachium novaehollandiae
  source 1..205: mol_type = genomic DNA
gtgggacgataagaccctataaaacttgatataagtctgaggggatttgtgatttcagtaaaaaatagtcctattaggtatatatttcgttggggagatgtagatataatgtgtaactgtctttaaaattgaataaatatagttagtttgtgatcctgtatttacggattaaaagagtaagttactttagggataacagcgtaat

SEQ ID NO: 1104 - DNA, 205 bp, Macrobrachium tolmerum
  source 1..205: mol_type = genomic DNA
atgggacgataagaccctataaaacttaatataagtctgatttaatttgtgatttaagtgaaaatcgattttgtttgacttatatttcgttggggagatgaagatataaagtgtaactgtctgttataatttaataatattaattagtttttgatccttctttgtggattaaaagaataagttactttagggataacagcgtaat

SEQ ID NO: 1105 - DNA, 204 bp, Macrobrachium iheringi
  source 1..204: mol_type = genomic DNA
aagggacgataagaccctataaaacttaatataaattttattttttatgtgatttaagtgaaactcagtttagtttgatttatatttcgttggggagatgtggatataaagtgtaactgtctgctgaatttaataattattgttagtgcgcgatccttctttgtgggttgtaagaataagttactttagggataacagcgtgat

SEQ ID NO: 1106 - DNA, 204 bp, Macrobrachium saigonense
  source 1..204: mol_type = genomic DNA
gtgggacgataagaccctataaaactttatataaaagtagtttggtttataatttaagtaaaaagtaactttgctaaatttatatttcgttggggagatgtagatataatgtgtaactgtctgtttaatttgataattgtagttagtatatgatccttctttgtggattaaaagtataagttactttagggataacagcgtgat

SEQ ID NO: 1107 - DNA, 204 bp, Macrobrachium nattereri
  source 1..204: mol_type = genomic DNA
gggggacgataagaccctataaaacttgatatagatttaattttgcttgtgatttcagtagagaggggttttgattagatcatgtttcgttggggagatgtggatataaagtgtaactgtctgtagaattatataattattattagtagatgatccttctttggggattagaagaataagttactttagggataacagcgtaat

SEQ ID NO: 1108 - DNA, 204 bp, Macrobrachium aracamuni
  source 1..204: mol_type = genomic DNA
gggggacgataagaccctataaaacttaatataaatttaatttattatatgatttaagtgaaaagtagtttaaggtagtttatattttgttggggtgatatagatataaaatgtaactgtctggaaaatttaataattttaattagtgtttgatccttctttgtggattaagagaataagttactttagggataacagcgtgat

SEQ ID NO: 1109 - DNA, 204 bp, Macrobrachium inpa
  source 1..204: mol_type = genomic DNA
gggggacgataagaccctgtaaaacttaatataaatttggttttatttgtgattttagtgaaaagaagtttaaattagtttatattttgttggggtgacatagatataaaatgtaactgtctaaaaaatttaataattttaattagtgtttgatccttctttggggatttaaagagtaagttactttagggataacagcgtaat

SEQ ID NO: 1110 - DNA, 204 bp, Macrobrachium depressimanum
  source 1..204: mol_type = genomic DNA
gggggacgataagaccctataaaactttatataaatttaatttagtttatgatataggtgaaaaattgttttaataattttatattttgttggggagacatagatataatatgtaactgtttgttaaatttaataattatagttaatatttgatccttccttgaggattaagagaataagttactttagggataacagcgtaat

SEQ ID NO: 1111 - DNA, 204 bp, Macrobrachium surinamicum
  source 1..204: mol_type = genomic DNA
gtgggacgataagaccctataaaacttgatataggttaagtttgatttgtgatttaagtgaagagtggttttgcttgacttatattttgctggggcggcatggatataaagtgtaactgtctattaaatttaataacattaattagtttctgatccttctttgtggattaaaagaataagttactttagggataacagcgtgat

SEQ ID NO: 1112 - DNA, 204 bp, Macrobrachium denticulatum
  source 1..204: mol_type = genomic DNA
gtgggacgataagaccctataaaacttgatataagttgaacttggtttgtgatttaggtgaaaaatagtcttgcttgacttatatttcgctggggcggcatagatataaaatgtaactgtctattaaatttaatagtgctaattagtctttgatccttctctgtggattaaaagaataagttactttagggataacagcgtgat

SEQ ID NO: 1113 - DNA, 201 bp, Macrobrachium pilimanus
  source 1..201: mol_type = genomic DNA
gtgggacgataagaccctataaaacttaatataggtttagtttgattcgcgattgtgggtgaaaagtagtgttgctagtcctatattttgttggggcgatgcggataaaatcggtaactgtttgtaaaatttaataattatgattgatccttcgtttgtgtggattaggagaataagttactttagggataacagcgtgat

SEQ ID NO: 1114 - DNA, 204 bp, Macrobrachium ohione
  source 1..204: mol_type = genomic DNA
gtgggacgataagaccctataaaacttgatattagtttaatttgatttgcgatttgagtgaagagcggttttatctatattatattttgttggggagacatagatataaacagtaactgtctattaaatttaatagcgttagttagtacttgatccttctttgtggattaaaagaataagttactttagggataacagcgtgat

SEQ ID NO: 1115 - DNA, 203 bp, Macrobrachium hainanense
  source 1..203: mol_type = genomic DNA
gtgggacgataagaccctataaaacttgatataagtttaattaacttataatttaagtgaaaagtattttaagtggttttatattttgttggggagatgtagatataatgtgtaactgtttataaaatttaataattataattagtatgtgatccttctttgtggattaaaagtataagttactttagggataacagcgtaat

SEQ ID NO: 1116 - DNA, 205 bp, Macrobrachium lepidactyloides
  source 1..205: mol_type = genomic DNA
gagggacgataagaccctataaaacttgacataatccaaatatgacttgagatttgagtgaagatcggtcccgtttggcttgcgtttcgttggggagatgaagatataaaatgtaactgtctgtttaaattaaatagtgttcagtagtttgtgatccttctctgtggattaaaagaataagttactttagggataacagcgtgat

SEQ ID NO: 1117 - DNA, 204 bp, Macrobrachium jaroense
  source 1..204: mol_type = genomic DNA
atgggacgataagaccctataaaacttaatatgatccgaccttgatttgtgatttgagtgaaaagtgatcttgtttggtttatatttcgttggggagatgaagatataaaatgtaactgtctatgaaatttaatagtgttaattagtttttgatccttctttgtggattaagagaataagttactttagggataacagcgtgat

SEQ ID NO: 1118 - DNA, 205 bp, Macrobrachium esculentum
  source 1..205: mol_type = genomic DNA
gtgggacgataagaccctataaaacttgatataagttttgcttgatttgtgatttatggtgaaaaatggttttgtttaacttatattttgttggggagatgtagatataatatgtaactgtctatttaatttaataattataattagtgtttgatccttctttgtggattaagagaataagttactttagggataacagcgtgat

SEQ ID NO: 1119 - DNA, 203 bp, Macrobrachium maculatum
  source 1..203: mol_type = genomic DNA
gtgggacgataagaccctataaaacttaatataaatttatttaatttatgatttaagtgaaaagtaatttaagtgggtttatattttgttggggagatgtagatataatatgtaactgtttattaaatttaataattataattagtgtgtgatccttctttgtggattaaaagtataagttactttagggataacagcgtgat

SEQ ID NO: 1120 - DNA, 204 bp, Macrobrachium edentatum
  source 1..204: mol_type = genomic DNA
gtgggacgataagaccctataaaacttgatataaatataatttaacttgtgatttaggtaaaaagtggttttattgggtttatatttcgttggggagatgtggatataatatgtaactgtctgtttaatttaataattatgattagtgtgtgatccttctttggggattaagagtttaagttactttagggataacagcgtgat

SEQ ID NO: 1121 - DNA, 206 bp, Macrobrachium grandimanus
  source 1..206: mol_type = genomic DNA
gagggacgataagaccctataaaactttacatgaattcagtttgattctgtgagctgatgtgaaaaacaatcctgcttaattcatgtttcgttggggagacgaatatataatttgtaactgtctgtttaattaaatagtgatagttagtatctgatccctctatgaggattaaaagaataagttactttagggataacagcgtgat

SEQ ID NO: 1122 - DNA, 203 bp, Macrobrachium malayanum
  source 1..203: mol_type = genomic DNA
gagggacgataagaccctataaaacttaatacaggtttgattaggttacgattttagtataaagtggtattattaagtttgtatttcgttggggcgatgaagatataatttgtaactgtctgttaaatataatagttattgctagaatttgatcctgctttgtggattataagaataagttactttagggataacagcgtaat

SEQ ID NO: 1123 - DNA, 204 bp, Macrobrachium meridionale
  source 1..204: mol_type = genomic DNA
atgggacgataagaccctataaaacttgatataggtttagcttggtttgtgatgtaagtgaaaagttgctttgcttggcttatatttcgttggggagatgaagatataaagtgtaactgtctattaaatttaatagtgctaattagtgtttgatccttctttgtggattaaaagaataagttactttagggataacagcgtgat

SEQ ID NO: 1124 - DNA, 204 bp, Macrobrachium neglectum
  source 1..204: mol_type = genomic DNA
gtgggacgataagaccctataaaacttaatataagttttgcttgatttatgatgtgtgtgaaaagtagttttgtttggtttatattttgttggggagatatagatataatttgtaactgtctatttaatttaatagttattgttagtgtgtgatccttctttgtggattaaaagaataagttactttagggataacagcgtgat

SEQ ID NO: 1125 - DNA, 206 bp, Macrobrachium platycheles
  source 1..206: mol_type = genomic DNA
gtgggacgataagaccctataaaacttgatataggtaaggcttggtttgtaattgtaggttaaaagttgttttgcttggcttatattttgttggggagatgtagatataatgtgtaactgtctgttgaatttaatagttataattagtgtgtgatccttcatttgtggattaggagaataagttactttagggataacagcgtgat

SEQ ID NO: 1126 - DNA, 204 bp, Macrobrachium naso
  source 1..204: mol_type = genomic DNA
gagggacgataagaccctataaaacttgatataaatttatcttaatttgtgatgtgagttaaaagtaattttgtttaaattatatttcgttggggagatgaagatataacatgtaactgtctgattaatttaataatcataattagtatttgatccttctttgtggattttaagaaaaagttactttagggataacagcgtgat

SEQ ID NO: 1127 - DNA, 205 bp, Macrobrachium placidum
  source 1..205: mol_type = genomic DNA
gggggacgataagaccctataaaactttatataagtttggtttagtctatgatttaggtgaaaagtggttttgtctagtttatatttcgttggggagatgaggatataaaatgtaactgtctgttttaatttaatagttctaactagtttgtgatccttctttggggattaaaagaataagttactttagggataacagcgtgat

SEQ ID NO: 1128 - DNA, 204 bp, Macrobrachium yui
  source 1..204: mol_type = genomic DNA
atgggacgataagaccctataaaacttaatataggtttagtttaatttgtaatttacgtaaaaaggggttttattaaatttatatttcgttggggagatgtggatataatttgtaactgtctgtagaatttaataattatagttagagtttgatccttcgttggggattagtagaataagttactttagggataacagcgtaat

SEQ ID NO: 1129 - DNA, 204 bp, Macrobrachium shokitai
  source 1..204: mol_type = genomic DNA
gggggacgataagaccctataaaactttatataaatttaatttaacttgtgagttaagtgaaaagtagttttgttgagtttatatttcgttggggagatgtagatataatatgtaactgtctatttaatttaataataatgattagtgtgtgatccttctttgtggattaaaagtttaagttactttagggataacagcgtgat

SEQ ID NO: 1130 - DNA, 205 bp, Macrobrachium sundaicum
  source 1..205: mol_type = genomic DNA
gtgggacgataagaccctataaaacttgatatgagtgaagtttagtttgtggtataggtggaaagtaattaaatttagtttatattttgttggggtgatgtagatataatgtgtaactgtctgttagaatttaataattataattagtgtttgatcctgctttgcggattaaaagaataagttactttagggataacagcgtaat

SEQ ID NO: 1131 - DNA, 205 bp, Macrobrachium rude
  source 1..205: mol_type = genomic DNA
gtgggacgataagaccctataaaacttgacacagacttagctgaatttgtgattttagtaaaaagtgattctgcgaagcctttgtttcgttggggagatgtagatataacttgtaactgtctataaaattaaataactgtgattagttgttgatccttcacttgtggattaaaagaataagttactttagggataacagcgtgat

SEQ ID NO: 1132 - DNA, 204 bp, Macrobrachium lamarrei
  source 1..204: mol_type = genomic DNA
aagggacgataagaccctataaaacttgatatagtttgaacttaacttgcggtttaagtgaaaagtagttttgtttgttttatattttgttggggagatagagatataatgtgtaactgtctattgaattttatagttttaattagtatttgatccttctttggggattaagagagtaagttactttagggataacagcgtgat

SEQ ID NO: 1133 - DNA, 204 bp, Macrobrachium sankolli
  source 1..204: mol_type = genomic DNA
gagggacgataagaccctataaaacttaatatagtttaaatttagctttcaacttaagtaaaaagtagttttgtttagtttatattttgttggggagatgaagatataatgtgtaactgtctattgaattttatagtgttaattagtatttgatccttctttggggattaagagaataagttactttagggataacagcgtgat

SEQ ID NO: 1134 - DNA, 205 bp, Macrobrachium gangeticum
  source 1..205: mol_type = genomic DNA
gggggacgataagaccctataaaactttatataatctaaacttgacttgcgatttaagtgaaaagtagttttgttcggtttatatttcgttggggagatgaagatataatttgtaactgtctattgaattttatagtattaaattagtatttgatccttctttgaggattaggagaataagttactttagggataacagcgtgat

SEQ ID NO: 1135 - DNA, 214 bp, Trachysalambria palaestinensis
  source 1..214: mol_type = genomic DNA
gggggacgataagaccctataaagctttacagttatttagttgaattataaattgttagtataaagttaatttaactaatgtttgtttcgttggggtgacgagaatataattttgtaattgttcttttatgttttattacaatgatttttggtataagaatgatcctttattaaagattaaaagattaagttactttagggataacagcgtaat

SEQ ID NO: 1136 - DNA, 211 bp, Euphausia pacifica
  source 1..211: mol_type = genomic DNA
aagtgacgataagaccctataaaacttgataaataaaattaaaaaagcgtattagtttattaaaactttttttctggaatttattttattggggcgataaaaatataaaaagtaactgtttttaaaattttaatggttaataactaaattaaataaatgatcctttaataaagattaaaagattaagttactttagggataacagcgtaat

SEQ ID NO: 1137 - DNA, 210 bp, Euphausia lucens
  source 1..210: mol_type = genomic DNA
aagtgacgataagaccctataaaacttaataagaaagataaaaaaagtgcagtagttaattaaaactttttttattaaacttattttattggggcgataaaaatgtaaaaagtaactgtttttaacttattaatgattatagtcagaaagattgaatgatcctttactaaagattataagattaagttactttagggataacagcgtaat

SEQ ID NO: 1138 - DNA, 210 bp, Euphausia vallentini
  source 1..210: mol_type = genomic DNA
aagtgacgataagaccctataaaacttaataagaaagataaaaaaagtgtagtagttaattaaaactttttttgttaaacttattttattggggcgataaaaatgtaaaaagtaactgtttttaacttcttaatgattatagtcagtaagattgaatgatcctttagtaaagattataagattaagttactttagggataacagcgtaat

SEQ ID NO: 1139 - DNA, 213 bp, Euphausia triacantha
  source 1..213: mol_type = genomic DNA
aagtgacgataagaccctataaaacttgataaataaaattaaaaaaggatattagttaaattaaaactttttttgtggaatttattttattggggcgataaaaatataattagtaactgtttttaatattttaatgattaataatcagagtttagtaaatgatcctctaatagagattataagattaagttactttagggataacagcgtgat

SEQ ID NO: 1140 - DNA, 212 bp, Euphausia longirostris
  source 1..212: mol_type = genomic DNA
aagtgacgataagaccctataaaacttgataaataaaattaaaaaatgatattagttaatttaaacttttttttatggtatttattttattggggcgataaaaatataataagtaactgtttttcatattttaatgataaataatcagagtaaataaatgatcctttaataaagattgtaagattaagttactttagggataacagcgtaat

SEQ ID NO: 1141 - DNA, 214 bp, Euphausia similis
  source 1..214: mol_type = genomic DNA
aagtgacgataagaccctataaaactttataaataaaataaaaaaaagatattagttgattgaaacttttttttatggaatttattttattggggcgataaaaatataataagtaactgtttttaatattataatgattaatagtcaaaatataaataaatgatcctttaataaagattgcaagattaagttactttagggataacagcgtaat

SEQ ID NO: 1142 - DNA, 213 bp, Euphausia recurve
  source 1..213: mol_type = genomic DNA
aagtgacgataagaccctataaaactttataaattaaactaaaaaaaaatattagttaattgaaattttttattgttgaatttattttattggggcgatacaaatataataagtaactgtttttcatatttaaatgataaataatcatgaacaattaaatgatcctttaataaagattgtaagattaagttactttagggataacagcgtaat

SEQ ID NO: 1143 - DNA, 213 bp, Euphausia krohni
  source 1..213: mol_type = genomic DNA
aagtgacgataagaccctataaaactttataaactaaactaaaaaaaaatattagttaattgaaattttttattgttgaatttattttattggggcgattaaaatataataagtaactgttttttatatttaaatgataattagtcatgagtaattaaatgatcctttagtaaagattgtaagattaagttactttagggataacagcgtaat

SEQ ID NO: 1144 - DNA, 210 bp, Euphausia frigida
  source 1..210: mol_type = genomic DNA
aagtgacgataagaccctataaaacttaataaggaaaacaaaaaaagtgcactagtttattaaagcttctcttgttaaacttattttattggggcgataaaaatgtaacaagtaactgtttttaacttcttaatgattatagtcagtaagattgaatgatcctttaataaagattataagattaagttactttagggataacagcgtaat

SEQ ID NO: 1145 - DNA, 212 bp, Euphausia gibboides
  source 1..212: mol_type = genomic DNA
aagtgacgataagaccctataaaacttgataaataaagttaaaaaaagatattagttaattgaaacttttttttgtggtgtttattttattggggcgattaaaatataataagtaactgttttttacatttttatgataaatagtcaagacaaataaatgatcctttattagagattataagattaagttactttagggataacagcgtaat

SEQ ID NO: 1146 - DNA, 206 bp, Euphausia eximia
  source 1..206: mol_type = genomic DNA
aggggacgataagaccctataaaactttatgcacgacatgtcctgtctttgaattatatccaaatattggttggctaaaagtgcattccgttggggagacgttgatataagctgtaactgtcttattaaatagaataattattattataatttgatccttctttgtggataaaaagaataagttactttagggataacagcgtaat

SEQ ID NO: 1147 - DNA, 213 bp, Euphausia americana
  source 1..213: mol_type = genomic DNA
aagtgacgataagaccctataaaactttataaattaaactaaaaaaaaatattagttaattgaaattttttattgttaaatttattttattggggcgatacaaatatattaagtaactgttttttatatttaaatgataaataatcataattaattaaatgatcctctattagagattgaaagattaagttactttagggataacagcgtaat

SEQ ID NO: 1148 - DNA, 210 bp, Euphausia tenera
  source 1..210: mol_type = genomic DNA
aagtgacgataagaccctataaaacttgataaataatattaaaaatgagcattagttagctaaatctttttttgtaaagtttattttattggggcgataaaaatataaatagtaactgtttttaaaattttaatggtagtaaacaaaaatattaaatgatcctttaataaagattataagattaagttactttagggataacagcgtaat

SEQ ID NO: 1149 - DNA, 209 bp, Euphausia pseudogibba
  source 1..209: mol_type = genomic DNA
aagtgacgataagaccctataaagcttaataaacaaaactaaaaataaatattagttaattaaatttttttttgtgtagtttattttattggggcgataaaaatataaatagtaactgtttttaatattttaatgataaataatcaaaaattaaatgatcctttaataaagattataagattaagctactttagggataacagcgtaat

SEQ ID NO: 1150 - DNA, 208 bp, Euphausia hemigibba
  source 1..208: mol_type = genomic DNA
aagtgacgataagaccctataaaacttgataaataaaattaaaaataaatattagttaattaaatttttttttgtgaagtttattttattggggcgataaaaatataaatagtaactgtttttaatattttaatgataatagtcaaaaattaaatgatcctttaataaagattataagattaagttactttagggataacagcgtgat

SEQ ID NO: 1151 - DNA, 212 bp, Euphausia brevis
  source 1..212: mol_type = genomic DNA
aagtgacgataagaccctataaaactttataaatttaaattaaaaaaagtattagtagtttaaaattttttattgttaaatttattttattggggcgatacaaatataataaataactgtttatatatttaaatgacgaataatcatgaataattaaatgatcctttaataaagattgtaagattaagttactttagggataacagcgtaat

SEQ ID NO: 1152 - DNA, 210 bp, Hymenopenaeus debilis
  source 1..210: mol_type = genomic DNA
aagggacgataagaccctataaagctttacaataaataaattaaattataaattattagtttaacttaatttaatttgtgtttgtttcgttggggcgacgagaatataataataactgttcttttataaaattaacaaaattaattggataataattgatcctttaataaagattaaaagattaagttactttagggataacagcgtaat

SEQ ID NO: 1153 - DNA, 208 bp, Nematopalaemon tenuipes
  source 1..208: mol_type = genomic DNA
gtgggacgataagaccctataaaactttatataaatggattgagcctgtgatttatctggtaaagcgggctgggtttttttatattttgttggggcgacattgataaaatttgttggtaactgttctgtgaattttataatggtggttaggtaaagatccttttatagggattaagagtttaagttactttagggataacagcgtaat